(19) United States
(12) Patent Application Publication
(10) Pub. No.: US 2024/0360134 A1
(43) Pub. Date: Oct. 31, 2024

(54) PYRAZOLOPYRIMIDINES AND THEIR USES AS PDGFR INHIBITORS

(71) Applicant: ACTELION PHARMACEUTICALS LTD, Allschwil (CH)

(72) Inventors: David BAUMAN, Raritan, NJ (US); Zhijie LIU, Raritan, NJ (US); Tianbao LU, Raritan, NJ (US)

(21) Appl. No.: 18/293,023

(22) PCT Filed: Jul. 29, 2022
(86) PCT No.: PCT/EP2022/071464

§ 371 Date: Jan. 29, 2024

Related U.S. Application Data
(60) Provisional application No. 63/227,654, filed on Jul. 30, 2021.
(60) Provisional application No. 63/354,389, filed on Jun. 22, 2022.

(51) Int. Cl. C07D 487/04; A61K 31/519; A61K 31/5365; A61K 31/5377; A61K 31/541; A61K 31/553; C07D 471/04; C07D 519/00

(52) U.S. Cl. CPC C07D 487/04; A61K 31/519; A61K 31/5365; A61K 31/5377; A61K 31/541; A61K 31/553; C07D 471/04; C07D 519/00

(57) ABSTRACT

The disclosure is directed to compounds of formula (I) and pharmaceutically acceptable salts thereof. Pharmaceutical compositions comprising compounds of formula (I), as well as methods of their use and preparation, are also described.

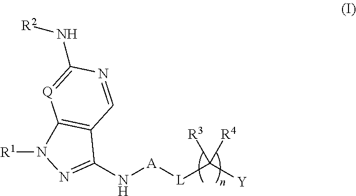

### CROSS-REFERENCE TO RELATED APPLICATIONS

This application claims the benefit of the priority of U.S. Provisional Patent Application No. 63/227,654, filed Jul. 30, 2021, and U.S. Provisional Patent Application No. 63/354,389, filed Jun. 22, 2022, the disclosures of which are incorporated by reference herein.

### TECHNICAL FIELD

The disclosure is directed to PDGFR inhibitors and methods of their use.

### BACKGROUND

Protein kinases are a family of enzymes that catalyze the phosphorylation of specific residues in proteins. Protein kinases play a critical role in the control cell growth, proliferation, differentiation, metabolism, apoptosis, cell mobility, transcription, translation and other signaling processes. The overexpression or inappropriate expression of protein kinases plays a significant role in the development of many diseases and disorders including central nervous system disorders, inflammatory disorders, metabolic disorders, autoimmune diseases, cardiovascular diseases, fibrotic diseases, transplantation rejection, cancer and infectious diseases.

Growth factors (GF) are important regulators of human homeostasis involved in maintaining a delicate balance between cell growth, differentiation, and proliferation. Consequently, dysregulation of GF signaling are implicated in many diseases including oncology, immunology, fibroproliferative, cardiovascular, vascular disorders and pulmonary hypertension. GF bind to several different receptors that amplify the signal through activation of the specific receptor through phosphorylation, leading to confirmation changes increasing the affinity for ATP and the phosphorylation of downstream proteins leading to activation of several signaling cascades. Therefore, small changes in GF or the cognate receptors can significantly alter the local signaling and have dramatic effects on initiation and progression of many diseases.

Platelet-derived growth factor (PDGF) is one of many GFs that regulate cell growth and division. PDGF exerts its biological responses via activation of two highly specific, transmembrane receptor tyrosine kinases, termed PDGFR α and PDGFR β, which can form three different dimeric receptors—αα, ββ and αβ. These receptors can interact with the different dimeric PDGF ligands (PDGF-AA, PDGF-BB, PDGF-CC, PDGF-DD and PDGF-AB) with different specificities and efficacies. The receptors are activated by ligand-induced dimerization, leading to autophosphorylation on specific tyrosine residues. PDGFR phosphorylation recruits signaling proteins containing Tyr(P)-binding domains. Several of these signaling proteins include Src kinase family members, phospholipase C-y1, the p38a subunit of PI3K, GTPase-activating protein. The formation of receptor-signaling complexes then initiates the activation of various signaling pathways, including the Ras-mitogen activated protein (MAP) kinase pathway, the PI3kinase-Akt pathway, the PLC-y1 and the Src pathway. Activation of PDGFRα or PDGFRβ by PDGFs, leads to protein synthesis, proliferation, migration, protection against apoptosis and cellular transformation, key mechanisms associated with several vascular diseases including pulmonary hypertension. Platelet-derived growth factor (PDGF) and its receptors (PDGFR), including PDGFRα and PDGFRβ, play important roles in tumorigenesis, tumor progression, and the regulation of stromal cell function. Constitutive activation of PDGFR signaling, gene rearrangement, and activating mutations of PDGFR have been identified in various types of human tumors and malignancies.

PDGFR signaling is implicated in the development and progression of pulmonary hypertension. PDGFs are expressed in ECs, SMCs and macrophages and are strong mitogens and chemokines. Increased signaling through PDGFRβ leads to smooth muscle cell proliferation which contributes to the development of vascular remodeling. PDGF and PDGF receptors (α and β) are upregulated in human and animals with pulmonary hypertension. Preclinically, efficacy in preventing and reversing vascular remodeling in experimentally induced pulmonary hypertension was demonstrated through non-selective inhibition of PDGF receptors. Clinically, imatinib (also known as Gleevec), a non-selective tyrosine kinase inhibitor including PDGF receptors improved exercise capacity and hemodynamics in patients with advanced pulmonary hypertension. Conversely, dasatinib, a receptor tyrosine kinases inhibitor, was linked to cardiotoxicity and the development of pulmonary hypertension, emphasizing the importance of the appropriate kinase selectivity, and associated differentiated profile.

A need exists for additional PDGFR inhibitors for the treatment of pulmonary hypertension and other conditions in which PDGFR signaling is implicated.

### SUMMARY

The present disclosure provides PDGFR inhibitors.

In particular, the present disclosure provides compounds of formula (I):

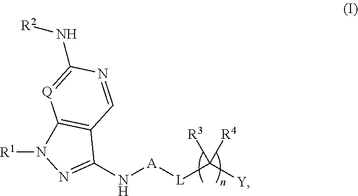

or a pharmaceutically acceptable salt thereof, wherein A is an optionally substituted phenyl ring, an optionally substituted pyridinyl ring, or an optionally substituted 5-membered heteroaryl ring containing 1-2 heteroatoms that are each independently O, N, or S; R¹ is an optionally substituted C₁-C₆alkyl group; R² is optionally substituted aryl, optionally substituted heteroaryl, optionally substituted cycloalkyl, optionally substituted C₁-C₆-alkyl-cycloalkyl, or optionally substituted heterocycloalkyl; Q is N or CH; n is 1, 2, or 3; R³ and R⁴ are each independently H, C₁-C₆alkyl, C₃-C₅cycloalkyl, or, when n is 2 or 3, one R³ and one R⁴ attached to the same carbon atom, together with that carbon atom, may form a C₃-C₆cycloalkyl ring and one R³ and one R⁴ attached to the same carbon atom, together with that carbon atom, may form a carbonyl group (>C═O); L is —C(O)NH—, —NHC(O)—, or —NHC(O)NH, wherein when n is 1 and Y is —NR⁵R⁶, then L is —NHC(O)—; Y is substituted or unsubstituted 3-7 membered cycloalkyl, optionally substituted 4-7 membered heterocycloalkyl, or —NR⁵R⁶; and R⁵ and R⁶ are each independently optionally substituted aryl, optionally substituted heteroaryl, optionally substituted alkyl, optionally substituted cycloalkyl, or optionally substituted heterocycloalkyl; or R⁵ and R⁶, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system optionally includes, in addition to the nitrogen atom to which both R⁵ and R⁶ are attached, 1-3 other heteroatoms that are each independently O, S, or N.

In some aspects, the disclosure is directed to compounds of formula (IA) or formula (IB):

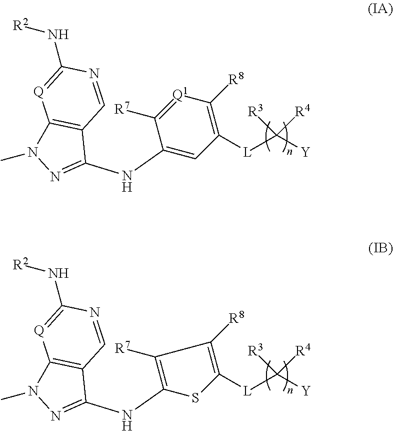

or a pharmaceutically acceptable salt thereof, wherein Q¹ is N or CH; R² is optionally substituted aryl, optionally substituted heteroaryl, optionally substituted cycloalkyl, optionally substituted C₁-C₆-alkyl-cycloalkyl, or optionally substituted heterocycloalkyl; Q is N or CH; n is 1, 2, or 3; R³ and R⁴ are each independently H, C₁-C₆alkyl, C₃-C₅cycloalkyl, or, when n is 2 or 3, one R³ and one R⁴ attached to the same carbon atom, together with that carbon atom, may form a C₃-C₆cycloalkyl ring and one R³ and one R⁴ attached to the same carbon atom, together with that carbon atom, may form a carbonyl group (>C═O); L is —C(O)NH—, —NHC(O)—, or —NHC(O)NH, wherein when n is 1 and Y is —NR⁵R⁶, then L is —NHC(O)—; Y is substituted or unsubstituted 3-7 membered cycloalkyl, optionally substituted 4-7 membered heterocycloalkyl, or —NR⁵R⁶; and R⁵ and R⁶ are each independently optionally substituted aryl, optionally substituted heteroaryl, optionally substituted alkyl, optionally substituted cycloalkyl, or optionally substituted heterocycloalkyl; or R⁵ and R⁶, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system optionally includes, in addition to the nitrogen atom to which both R⁵ and R⁶ are attached, 1-3 other heteroatoms that are each independently O, S, or N; R⁷ is H, C₁-C₆alkyl, C₃-C₆cycloalkyl, halogen, —CN, or C₁-C₄fluoroalkyl; and R⁸ is H, C₁-C₆alkyl, C₃-C₆cycloalkyl, halogen, or C₁-C₄fluoroalkyl.

Pharmaceutical compositions comprising such compounds, and methods of using such compounds in treating conditions in which PDGFR signaling is implicated are also provided.

The disclosure also provides compounds of formula (I), or a pharmaceutically acceptable salt thereof, for use as a medicament.

The disclosure further provides compounds of formula (I), or a pharmaceutically acceptable salt thereof, for use in treating a disease or disorder in which PDGFR signaling is implicated in a subject in need thereof.

The disclosure further provides uses of compounds of formula (I), or a pharmaceutically acceptable salt thereof, in the manufacture of a medicament for treating a disease or disorder in which PDGFR signaling is implicated in a subject in need thereof.

### DETAILED DESCRIPTION OF ILLUSTRATIVE EMBODIMENTS

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (e.g., to at least one) of the grammatical object of the article unless the context clearly indicates otherwise. By way of example, “an element” means one element or more than one element.

The term “compound”, as used herein, unless otherwise indicated, refers to any specific chemical compound disclosed herein and includes tautomers, optical isomers (enantiomers) and other stereoisomers (diastereomers) thereof, as well as pharmaceutically acceptable salts and derivatives, including prodrug and/or deuterated forms thereof where applicable. Deuterated small molecules contemplated are those in which one or more of the hydrogen atoms contained in the drug molecule have been replaced by deuterium. It is understood by those of ordinary skill that molecules which are described herein are stable compounds as generally described hereunder.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith.

A “solvate” refers to a physical association of a compound of formula (I) with one or more solvent molecules.

The term “alkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group. Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. In some embodiments, the alkyl group is a C₁-C₆ alkyl; in some embodiments, it is a C₁-C₄ alkyl.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C₁, C₂, and C₃.

The term “cycloalkyl” when used alone or as part of a substituent group refers to cyclic-containing, non-aromatic hydrocarbon groups having from 3 to 10 carbon atoms (“C₃-C₁₀”), preferably from 3 to 6 carbon atoms (“C₃-C₆”). Examples of cycloalkyl groups include cyclopropyl, cyclobutyl, cyclopentyl, cyclohexyl, cycloheptyl, cyclooctyl, indenyl, bicyclo[2.2.1]heptanyl, bicyclo[3.1.1]heptanyl, bicyclo[4.1.0]heptanyl, spiro[3.3]heptanyl, and spiro[3.4]octanyl.

The term “fluoroalkyl” when used alone or as part of a substituent group refers to an alkyl group wherein one or more of the hydrogen atoms has been replaced with one or more fluorine atoms. Examples of fluoroalkyl groups include —CF₃, CHF₂, —CH₂F and the like.

The term “heterocycloalkyl” when used alone or as part of a substituent group refers to any three to twelve-membered monocyclic, saturated or partially unsaturated ring containing at least one heteroatom that is O, N or S. The heterocycloalkyl group may be attached at any heteroatom or carbon atom of the ring such that the result is a stable structure. Examples of heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, tetrahydrofuranyl, tetrahydropyranyl, and the like.

The term “bridged heterocycloalkyl ring” refers to any 5 to 12 membered heterocycloalkyl ring system that contains at least one bridged ring. Examples of bridged heterocycloalkyl rings include azabicyclo[3.1.1]heptane, azabicyclo[3.1.1]heptane, azabicyclo[2.2.2]octane, azabicyclo[2.2.1]heptane, azabicyclo[2.1.1]hexane, azabicyclo[1.1.1]pentane, azabicyclo[1.1.1]pentane, 6-oxa-azabicyclo[3.1.1]heptane, 6-diazabicyclo[3.1.1]heptane, 3-thia-azabicyclo[3.1.1]heptane, and the like.

The term “fused heterocycloalkyl ring system” refers to a heterocycloalkyl ring to which another ring is fused. The other ring that is fused to the heterocycle ring may be another heterocycloalkyl ring, a cycloalkyl ring, an aryl ring, or a heteroaryl ring. In some embodiments, the fused heterocycloalkyl ring system is a 4 to 12 membered fused heterocycloalkyl ring system.

The term “spiroheterocycloalkyl ring system” refers to a heterocycloalkyl ring that is substituted with a spirocyclic ring. The spirocyclic ring can be a cycloalkyl ring or a heterocycloalkyl ring. In some embodiments, the spiroheterocycloalkyl ring system is a 5-12-membered spiroheterocycloalkyl ring system. The term “spirocyclic ring” refers to a cycloalkyl or heterocycloalkyl ring that shares one carbon atom with another cyclic ring.

The terms “halo” or “halogen”, by itself or as part of another substituent, means a fluorine, chlorine, bromine, or iodine atom.

The term “aryl” when used alone or as part of a substituent group also refers to a mono- or bicyclic-aromatic hydrocarbon ring structure having 6 or 10 carbon atoms in the ring, wherein one or more of the carbon atoms in the ring is optionally substituted. The term “aryl” also includes a mono- or bicyclic-aromatic hydrocarbon ring structure having 6 or 10 carbon atoms in the ring, wherein two adjacent carbon atoms in the ring are optionally substituted such that said two adjacent carbon atoms and their respective substituents form a cycloalkyl or heterocycloalkyl ring. Non-limiting examples of aryl groups include, but are not limited to, phenyl, indenyl, naphthyl, 1, 2, 3,4-tetrahydronaphthyl, and the like.

The term “heteroaryl” when used alone or as part of a substituent group refers to a mono- or bicyclic-aromatic ring structure including carbon atoms as well as up to four heteroatoms that are each independently nitrogen, oxygen, or sulfur. Heteroaryl rings can include a total of 5, 6, 9, or 10 ring atoms. The heteroaryl moiety can be unsubstituted, or one or more of the carbon atoms in the ring can be substituted. Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl, benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole.

The term “optionally substituted,” or “substituted or unsubstituted” as used herein to describe a substituent defined herein, means that the substituent may, but is not required to be, substituted with one or more suitable functional groups or other substituents as provided herein. For example, a substituent may be optionally substituted with one or more of: halo (i.e., —F, —Cl, —Br, —I), —OH, cyano, —C₁-C₆alkyl, C₃-C₆cycloalkyl, C₂-C₆alkenyl, C₂-C₆ alkynyl, C₁-C₆ haloalkyl, —C₁-C₆alkoxy, —C₁-C₆haloalkoxy, —C₁-C₆ alkylthio, —C₁-C₆ alkylamino, —NH₂, —NH(C₁-C₆ alkyl), —N(C₁-C₆ alkyl)₂, —NH(C₁-C₆ alkoxy), —C(O)NHC₁-C₆ alkyl, —C(O)N(C₁-C₆ alkyl)₂, —COOH, —C₁-C₆alkylCOOH, C₃-C₆cycloalkylCOOH, —C₁-C₆alkylCOOC₁-C₆alkyl, —C₃-C₆cycloalkylCOOC₁-C₆alkyl, —C(O)NH₂, —C₁-C₆alkylCONH₂, —C₃-C₆cycloalkylCONH₂, C₁-C₆alkylCONHC₁-C₆alkyl, —C₁-C₆alkylCON(C₁-C₆alkyl)₂, —C(O)C₁-C₆ alkyl, —C(O)OC₁-C₆ alkyl, —NHCO(C₁-C₆ alkyl), —N(C₁-C₆ alkyl)C(O)(C₁-C₆ alkyl), —S(O)C₁-C₆ alkyl, —S(O)₂C₁-C₆ alkyl, oxo, 3-6 membered heterocycloalkyl, 6-12 membered aryl, or 5 to 12 membered heteroaryl groups. In some embodiments, each of the above optional substituents are themselves optionally substituted by one or two groups.

In some embodiments, the term “optionally substituted,” or “substituted or unsubstituted” as used herein includes a —C₁-C₆alkyl-O—C₁-C₆alkyl group.

As used herein, the term “alkenyl” refers to a straight- or branched-chain group having from 2 to 12 carbon atoms (“C₂_C₁₂”), preferably 2 to 4 carbons atoms (“C₂-C₄”), in the group, wherein the group includes at least one carbon-carbon double bond. Examples of alkenyl groups include vinyl (—CH═CH₂; C₂alkenyl) allyl (—CH₂— CH═CH₂; C₃alkenyl), propenyl (—CH═CHCH₃; C₃alkenyl); isopropenyl (—C(CH₃)=CH₂; C₃alkenyl), butenyl (—CH═CHCH₂CH₃; C₄alkenyl), sec-butenyl (—C(CH₃)=CHCH₃; C₄alkenyl), iso-butenyl (—CH═C(CH₃)₂; C₄alkenyl), 2-butenyl (—CH₂CH═CHCH₃; C₄alkyl), pentenyl (CH═CHCH₂CH₂CH₃; C₅alkenyl), and the like.

As used herein, the term “alkynyl” refers to a straight- or branched-chain group having from 1 to 12 carbon atoms (“C₁-C₁₂”), preferably 1 to 4 carbons atoms (“C₂-C₄”), in the group, and wherein the group includes at least one carbon-carbon triple bond. Examples of alkynyl groups include ethynyl (—C≡CH; C₂alkynyl); propargyl (—CH₂—C≡CH; C₃alkynyl), propynyl (—C≡CCH₃; C₃alkynyl); butynyl (—C≡CCH₂CH₃; C₄alkynyl), pentynyl (C≡CCH₂CH₂CH₃; C₅alkynyl), and the like.

As used herein, the term “alkoxy” refers to an oxygen radical attached to an alkyl group by a single bond. Examples of alkoxy groups include methoxy (—OCH₃), ethoxy (—OCH₂CH₃), isopropoxy (—OCH(CH₃)₂) and the like.

As used herein, the term “haloalkoxy” refers to an oxygen radical attached to a haloalkyl group by a single bond. Examples of haloalkoxy groups include —OCF₃, —OCH₂CF₃, —OCH(CF₃)₂, and the like.

The term “haloalkyl” refers to an alkyl group wherein one or more of the hydrogen atoms has been replaced with one or more halogen atoms.

The term “haloalkoxy” refers to an alkoxy group wherein one or more of the hydrogen atoms has been replaced with one or more halogen atoms.

As used herein, the term “stereoisomers” refers to compounds which have identical chemical constitution, but differ with regard to the arrangement of the atoms or groups in space, e.g., enantiomers, diastereomers or tautomers.

The term “patient” or “subject” is used throughout the specification to describe an animal, preferably a human or a domesticated animal, to whom treatment, including prophylactic treatment, with the compositions according to the present disclosure is provided. For treatment of those conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

In some aspects, the present disclosure provides compounds of formula (I):

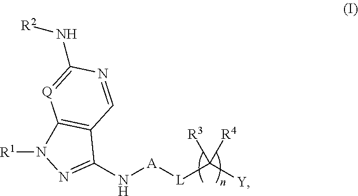

or a pharmaceutically acceptable salt thereof, wherein A is an optionally substituted phenyl ring, an optionally substituted pyridinyl ring, or an optionally substituted 5-membered heteroaryl ring containing 1-2 heteroatoms that are each independently O, N, or S; R¹ is an optionally substituted C₁-C₆alkyl group; R² is optionally substituted aryl, optionally substituted heteroaryl, optionally substituted cycloalkyl, optionally substituted C₁-C₆-alkyl-cycloalkyl, or optionally substituted heterocycloalkyl; Q is N or CH; n is 1, 2, or 3; R³ and R⁴ are each independently H, C₁-C₆alkyl, C₃-C₅cycloalkyl, or, when n is 2 or 3, one R³ and one R⁴ attached to the same carbon atom, together with that carbon atom, may form a C₃-C₆cycloalkyl ring and one R³ and one R⁴ attached to the same carbon atom, together with that carbon atom, may form a carbonyl group (>C═O); L is —C(O)NH—, —NHC(O)—, or —NHC(O)NH, wherein when n is 1 and Y is —NR⁵R⁶, then L is —NHC(O)—; Y is substituted or unsubstituted 3-7 membered cycloalkyl, optionally substituted 4-7 membered heterocycloalkyl, or —NR⁵R⁶; R⁵ and R⁶ are each independently optionally substituted aryl, optionally substituted heteroaryl, optionally substituted alkyl, optionally substituted cycloalkyl, or optionally substituted heterocycloalkyl; or R⁵ and R⁶, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system optionally includes, in addition to the nitrogen atom to which both R⁵ and R⁶ are attached, 1-3 other heteroatoms that are each independently O, S, or N.

In some aspects, A in the compound of formula (I) is an optionally substituted phenyl ring, an optionally substituted pyridinyl ring, or an optionally substituted 5-membered heteroaryl ring containing 1-2 heteroatoms that are each independently O, N, or S.

In some embodiments, A in formula (I) is an optionally substituted phenyl ring.

In other embodiments, A in formula (I) is an optionally substituted pyridinyl ring.

In some embodiments, A in formula (I) is an optionally substituted 5-membered heteroaryl ring containing 1-2 heteroatoms that are each independently O, N, or S, such as, for example, furan, pyrrole, thiophene, isoxazole, oxazole, pyrazole, imidazole, isothiazole, thiazole, and the like.

In some embodiments, A in formula (I) is an optionally substituted thiophene.

In some aspects, R¹ in the compounds of formula (I) is an optionally substituted C₁-C₆alkyl group, such as, for example, methyl, ethyl, n-propyl, iso-propyl, n-butyl, t-butyl, iso-butyl, sec-butyl, n-pentyl, n-hexyl, and the like.

In some embodiments, R¹ in the compounds of formula (I) is methyl (i.e., —CH₃).

In some aspects, R² in the compounds of formula (I) is optionally substituted aryl, optionally substituted heteroaryl, optionally substituted cycloalkyl, optionally substituted C₁-C₆-alkyl-cycloalkyl, or optionally substituted heterocycloalkyl.

In some embodiments, R² in the compounds of formula (I) is optionally substituted aryl, such as, for example, phenyl, indenyl, naphthyl, 1, 2, 3,4-tetrahydronaphthyl, and the like.

In some embodiments, R² in the compounds of formula (I) is substituted aryl.

In some embodiments, R² in the compounds of formula (I) is unsubstituted phenyl.

In some embodiments, R² in the compounds of formula (I) is substituted phenyl.

In some embodiments, R² in the compounds of formula (I) is phenyl substituted with —C₁-C₆alkylCOOH, C₃-C₆cycloalkylCOOH, —C₁-C₆alkylCOOC₁-C₆alkyl, C₃-C₆cycloalkylCOOC₁-C₆alkyl, or —NHCO(C₁-C₆ alkyl).

In some embodiments, R² in the compounds of formula (I) is:

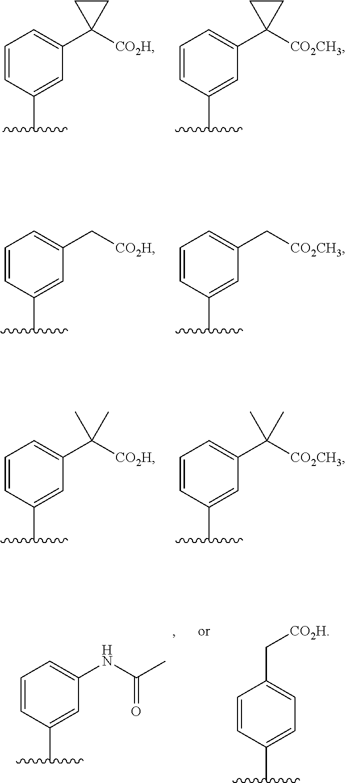

In some embodiments, R² in the compounds of formula (I) is optionally substituted heteroaryl, such as, for example, an optionally substituted pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl, benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole.

In other embodiments, R² in the compounds of formula (I) is an optionally substituted heteroaryl that is 6,7-dihydropyrazolo[5,1-b][1,3]oxazin-3-yl.

In some embodiments, R² in the compounds of formula (I) is substituted heteroaryl.

In some embodiments of R², the substituted heteroaryl is substituted with an optionally substituted C₁-C₆alkyl, such as, for example, methyl, ethyl, n-propyl, iso-propyl, n-butyl, t-butyl, iso-butyl, sec-butyl, n-pentyl, n-hexyl, and the like; or an optionally substituted C₃-C₅cycloalkyl, such as, for example, cyclopropyl, cyclobutyl, cyclopentyl, and the like.

In some embodiments of R², the optionally substituted heteroaryl is an optionally substituted 5-membered heteroaryl.

In some embodiments of R², the optionally substituted 5-membered heteroaryl is substituted with an optionally substituted C₁-C₆alkyl, such as, for example, methyl, ethyl, n-propyl, iso-propyl, n-butyl, t-butyl, iso-butyl, sec-butyl, n-pentyl, n-hexyl, and the like; or an optionally substituted C₃-C₅cycloalkyl, such as, for example, cyclopropyl, cyclobutyl, cyclopentyl, and the like.

In other embodiments of R², the optionally substituted 5-membered heteroaryl is substituted with an optionally substituted 3- to 5-membered heterocycloalkyl, such as, for example, oxiranyl, oxetanyl, or tetrahydrofuranyl.

In some embodiments of R², the optionally substituted 5-membered heteroaryl is an is an optionally substituted pyrazolyl.

In some embodiments of R², the optionally substituted pyrazolyl is substituted with an optionally substituted C₁-C₆alkyl, such as, for example, methyl, ethyl, n-propyl, iso-propyl, n-butyl, t-butyl, iso-butyl, sec-butyl, n-pentyl, n-hexyl, and the like; or an optionally substituted C₃-C₅cycloalkyl, such as, for example, cyclopropyl, cyclobutyl, cyclopentyl, and the like.

In some embodiments of R², the optionally substituted pyrazolyl is substituted with a methyl group, i.e., —CH₃.

In some embodiments of R², the optionally substituted pyrazolyl is substituted with a 2-hydroxyethyl group, i.e., —CH₂CH₂OH.

In some embodiments of R², the optionally substituted pyrazolyl is substituted with a 2-methoxyethyl group, i.e., —CH₂CH₂OCH₃.

In other embodiments of R², the optionally substituted pyrazolyl is substituted with a cyclopropyl group.

In other embodiments of R², the optionally substituted pyrazolyl is substituted with an oxetanyl group.

In some embodiments of R², the optionally substituted pyrazolyl is 1-methyl-pyrazol-3-yl, 1-methyl-pyrazol-4-yl, 1-methyl-pyrazol-5-yl, 1-cyclopropyl-pyrazol-3-yl, 1-cyclopropyl-pyrazol-4-yl, 1-cyclopropyl-pyrazol-5-yl, 1-(2-hydroxyethyl)-pyrazol-3-yl, 1-(2-hydroxyethyl)-pyrazol-4-yl, or 1-(2-hydroxyethyl)-pyrazol-5-yl.

In some embodiments of R², the optionally substituted pyrazolyl is 1,5-dimethyl-pyrazol-4-yl, 1,3-dimethyl-pyrazol-4-yl, or 1,3-dimethyl-pyrazol-5-yl.

In some embodiments of R², the optionally substituted pyrazolyl is 1-(2-methoxyethyl)-pyrazol-4-yl.

In some embodiments of R², the optionally substituted pyrazolyl is 1-(oxetan-3-yl)-pyrazol-4-yl.

In some embodiments of R², the optionally substituted 5-membered heteroaryl is an is an optionally substituted imidazolyl.

In some embodiments of R², the optionally substituted imidazolyl is substituted with an optionally substituted C₁-C₆alkyl, such as, for example, methyl, ethyl, n-propyl, iso-propyl, n-butyl, t-butyl, iso-butyl, sec-butyl, n-pentyl, n-hexyl, and the like; or an optionally substituted C₃-C₅cycloalkyl, such as, for example, cyclopropyl, cyclobutyl, cyclopentyl, and the like.

In some embodiments of R², the optionally substituted imidazolyl is substituted with a methyl group, i.e., —CH₃.

In some embodiments of R², the optionally substituted imidazolyl is 1-methyl-imidazol-4-yl. In some embodiments, R² in the compounds of formula (I) is an optionally substituted 6-membered heteroaryl.

In some embodiments of R², the optionally substituted 6-membered heteroaryl is substituted with an optionally substituted C₁-C₆alkyl, such as, for example, methyl, ethyl, n-propyl, iso-propyl, n-butyl, t-butyl, iso-butyl, sec-butyl, n-pentyl, n-hexyl, and the like; or an optionally substituted C₃-C₅cycloalkyl, such as, for example, cyclopropyl, cyclobutyl, cyclopentyl, and the like.

In other embodiments of R², the optionally substituted 6-membered heteroaryl is substituted with a —OC₁-C₆alkyl, such as, for example, —OCH₃.

In some embodiments of R², the optionally substituted 6-membered heteroaryl is an is an optionally substituted pyridinyl.

In some embodiments of R², the optionally substituted pyridinyl is substituted with an optionally substituted C₁-C₆alkyl, such as, for example, methyl, ethyl, n-propyl, iso-propyl, n-butyl, t-butyl, iso-butyl, sec-butyl, n-pentyl, n-hexyl, and the like; or an optionally substituted C₃-C₅cycloalkyl, such as, for example, cyclopropyl, cyclobutyl, cyclopentyl, and the like.

In other embodiments of R², the optionally substituted pyridinyl is substituted with an optionally substituted —OC₁-C₆alkyl, such as, for example, —OCH₃.

In some embodiments of R², the optionally substituted optionally substituted pyridinyl is unsubstituted pyridinyl.

In some embodiments, R² in the compounds of formula (I) is substituted: pyridinyl, pyrazolyl, pyridazinyl, or 1,2,3-triazolyl.

In some embodiments, R² in the compounds of formula (I) is substituted pyridinyl.

In some embodiments, R² in the compounds of formula (I) is pyridinyl substituted with —OH, —NH₂, —COOH, —C₁-C₆alkyl-COOH, —C(O)NH₂, —NHCO(C₁-C₆ alkyl), or optionally substituted 3-7 membered heterocycloalkyl.

In some embodiments, R² in the compounds of formula (I) is pyridinyl substituted with —CH₃ or —OCH₃.

In some embodiments, R² in the compounds of formula (I) is:

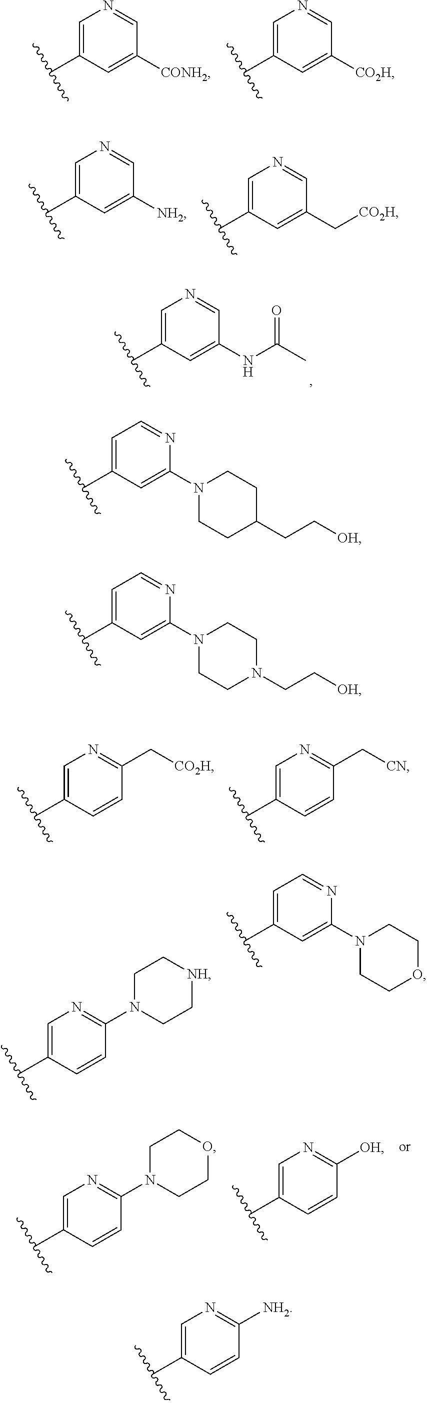

In some embodiments, R² in the compounds of formula (I) is:

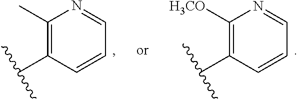

In some embodiments, R² in the compounds of formula (I) is substituted pyridazinyl.

In some embodiments, R² in the compounds of formula (I) is:

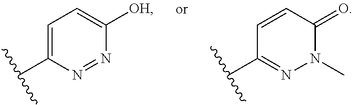

It will be apparent to those of skill in the art that some hydroxy-substituted heterocyclic moieties may exist in tautomeric forms. The disclosure encompasses all such tautomeric forms. Thus, for example, the moieties may exist as the tautomers below:

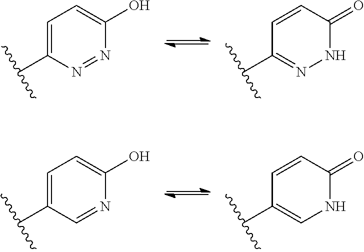 [SMILES: *c1ccc(O)nn1 ; *c1ccc(=O)[nH]n1 ; *c1ccc(O)nc1 ; *c1ccc(=O)[nH]c1]

In some embodiments, R² in the compounds of formula (I) is substituted pyrazolyl.

In some embodiments, R² in the compounds of formula (I) is N-methyl pyrazolyl.

In some embodiments, R² in the compounds of formula (I) is

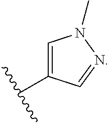 [SMILES: *c1cnn(C)c1]

In some embodiments, R² in the compounds of formula (I) is substituted: 1,2,3-triazolyl.

In some embodiments, R² in the compounds of formula (I) is

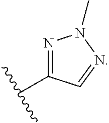 [SMILES: *c1cnn(C)n1]

In some embodiments, R² in the compounds of formula (I) is unsubstituted heteroaryl.

In some embodiments, R² in the compounds of formula (I) is unsubstituted: pyrimidinyl, pyridazinyl, or pyridinyl.

In some embodiments, R² in the compounds of formula (I) is unsubstituted: pyrimidinyl.

In some embodiments, R² in the compounds of formula (I) is

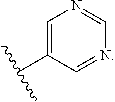 [SMILES: *c1cncnc1]

In some embodiments, R² in the compounds of formula (I) is unsubstituted pyridazinyl.

In some embodiments, R² in the compounds of formula (I) is

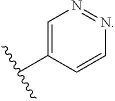 [SMILES: *c1ccnnc1]

In some embodiments, R² in the compounds of formula (I) is unsubstituted pyridinyl.

In some embodiments, R² in the compounds of formula (I) is

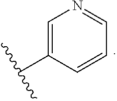 [SMILES: *c1cccnc1]

In other embodiments, R² in the compounds of formula (I) is

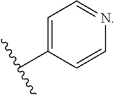 [SMILES: *c1ccncc1]

In some embodiments, R² in the compounds of formula (I) is optionally substituted cycloalkyl, such as, for example, cyclopropyl, cyclobutyl, cyclopentyl, cyclohexyl and the like.

In some embodiments, R² in the compounds of formula (I) is optionally substituted C₁-C₆alkyl-cycloalkyl, such as, for example, C₆alkyl-cycloalkyl, C₅alkyl-cycloalkyl, C₄alkyl-cycloalkyl, C₃alkyl-cycloalkyl, C₂alkyl-cycloalkyl, C₁alkyl-cycloalkyl.

In some embodiments, R² in the compounds of formula (I) is

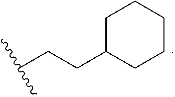 [SMILES: *CCC1CCCCC1]

In some embodiments, R² in the compounds of formula (I) is optionally substituted heterocycloalkyl.

In some embodiments, R² in the compounds of formula (I) is unsubstituted heterocycloalkyl.

In some embodiments, R² in the compounds of formula (I) is tetrahydro-2H-thiopyranyl 1,1-dioxide.

In some embodiments, R² in the compounds of formula (I) is

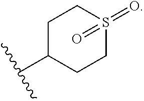

In some aspects, Q in the compounds of formula (I) is N or CH.

In some embodiments, Q in the compounds of formula (I) is N.

In some embodiments, Q in the compounds of formula (I) is CH.

In some aspects, n in the compounds of formula (I) is 1, 2, or 3.

In some embodiments, n in the compounds of formula (I) is 1.

In some embodiments, n in the compounds of formula (I) is 2.

In some embodiments, n in the compounds of formula (I) is 3.

In some aspects, when n is 1 in the compounds of formula (I), L is —NHC(O)—, and when n is 2 or 3 in the compounds of formula (I), L is —C(O)NH—, —NHC(O)—, or —NHC(O)NH.

In some embodiments of the compounds of formula (I), n is 2 or 3 and L is —C(O)NH—.

In some embodiments of the compounds of formula (I), n is 2 and L is —C(O)NH—.

In some embodiments of the compounds of formula (I), n is 3 and L is —C(O)NH—.

In other embodiments of the compounds of formula (I), n is 1 and L is —NHC(O)—.

In other embodiments of the compounds of formula (I), n is 2 and L is —NHC(O)—.

In other embodiments of the compounds of formula (I), n is 3 and L is —NHC(O)—.

In other embodiments of the compounds of formula (I), n is 2 or 3 and L is —NHC(O)NH—.

In other embodiments of the compounds of formula (I), n is 2 and L is —NHC(O)NH—.

In other embodiments of the compounds of formula (I), n is 3 and L is —NHC(O)NH—.

In some aspects R³ and R⁴ are each independently H, C₁-C₆alkyl, C₃-C₅cycloalkyl, or, when n is 2 or 3, one R³ and one R⁴ attached to the same carbon atom, together with that carbon atom, may form a C₃-C₆cycloalkyl ring and one R³ and one R⁴ attached to the same carbon atom, together with that carbon atom, may form a carbonyl group (>C═O).

In some embodiments, R³ or R⁴ in the compounds of formula (I) is H.

In some embodiments, each R³ and each R⁴ in the compounds of formula (I) is H.

In some embodiments, R³ or R⁴ in the compounds of formula (I) is C₁-C₆alkyl, such as, for example, methyl, ethyl, n-propyl, iso-propyl, n-butyl, t-butyl, iso-butyl, sec-butyl, n-pentyl, n-hexyl, and the like.

In some embodiments, R³ or R⁴ in the compounds of formula (I) is C₃-C₅cycloalkyl, such as, for example, cyclopropyl, cyclobutyl, cyclopentyl, and the like.

In some embodiments, when n is 2 or 3, one R³ and one R⁴ attached to the same carbon atom, together with that carbon atom, form a C₃-C₆cycloalkyl ring such as, for example, cyclopropyl, cyclobutyl, cyclopentyl, cyclohexyl, and the like.

In some embodiments, when n is 2 or 3, one R³ and one R⁴ attached to the same carbon atom, together with that carbon atom, form a carbonyl group (>C=O).

In some embodiments, when n is 2 or 3, one R³ and one R⁴ attached to the same carbon atom, together with that carbon atom, form a C₃-C₆cycloalkyl ring, and one R³ and one R⁴ attached to the same carbon atom, together with that carbon atom, may form a carbonyl group (>C═O).

In some embodiments, when n is 2 or 3, one R³ and one R⁴ attached to the same carbon atom, together with that carbon atom, form a cyclopropyl ring, and one R³ and one R⁴ attached to the same carbon atom, together with that carbon atom, may form a carbonyl group (>C═O).

In some aspects, Y in the compounds of formula (I) is substituted or unsubstituted 3-7-membered cycloalkyl, optionally substituted 4-7 membered heterocycloalkyl, or —NR⁵R⁶.

In some embodiments, Y is a substituted or unsubstituted 3-7-membered cycloalkyl, such as, for example, substituted or unsubstituted: 3-membered cycloalkyl, 4-membered cycloalkyl, 5-membered cycloalkyl, 6-membered cycloalkyl, or 7-membered cycloalkyl.

In some embodiments, Y is cyclohexyl.

In some embodiments, Y is an optionally substituted 4-7 membered heterocycloalkyl, such as, for example, 4-membered heterocycloalkyl, 5-membered heterocycloalkyl, 6-membered heterocycloalkyl, or 7-membered heterocycloalkyl. In such embodiments, the 4-7 membered heterocycloalkyl is not bound to the R³, R⁴-substituted carbon atom through a nitrogen atom.

In some embodiments, Y is —NR⁵R⁶.

In some aspects, R⁵ and R⁶ in the compounds of formula (I) are each independently optionally substituted aryl, optionally substituted heteroaryl, optionally substituted alkyl, optionally substituted cycloalkyl, or optionally substituted heterocycloalkyl; or R⁵ and R⁶, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system may include, in addition to the nitrogen atom to which both R⁵ and R⁶ are attached, 1-3 other heteroatoms that are each independently O, S, or N.

In some embodiments, R⁵ or R⁶ in the compounds of formula (I) is optionally substituted aryl, such as, for example, optionally substituted phenyl, indenyl, naphthyl, or 1,2,3,4-tetrahydronaphthyl.

In some embodiments, R⁵ or R⁶ in the compounds of formula (I) is optionally substituted heteroaryl, such as, for example, optionally substituted pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole.

In some embodiments, R⁵ or R⁶ in the compounds of formula (I) is optionally substituted alkyl, such as, for example, optionally substituted C₁-C₆alkyl, for example, methyl, ethyl, n-propyl, iso-propyl, n-butyl, t-butyl, iso-butyl, sec-butyl, n-pentyl, n-hexyl, and the like.

In some embodiments, R⁵ or R⁶ in the compounds of formula (I) is —CH₃.

In some embodiments, R⁵ or R⁶ in the compounds of formula (I) is —CH(CH₃)₂.

In some embodiments, R⁵ or R⁶ in the compounds of formula (I) is —CH₂CH₂OH.

In some embodiments, R⁵ or R⁶ in the compounds of formula (I) is —CH₂CH₂CH₂OH.

In some embodiments, R⁵ or R⁶ in the compounds of formula (I) is —CH₂CH₂OCH₃.

In some embodiments, R⁵ or R⁶ in the compounds of formula (I) is —CH₂CH₂CH₂OCH₃.

In some embodiments, R⁵ or R⁶ in the compounds of formula (I) is —CH₂CH₂F.

In some embodiments, R⁵ or R⁶ in the compounds of formula (I) is optionally substituted cycloalkyl, such as, for example, optionally substituted C₃-C₆cycloalkyl, for example, cyclopropyl, cyclobutyl, cyclopentyl, cyclohexyl, and the like.

In some embodiments, R⁵ or R⁶ in the compounds of formula (I) is cyclopentyl.

In some embodiments, R⁵ or R⁶ in the compounds of formula (I) is cyclobutyl.

In some embodiments, R⁵ or R⁶ in the compounds of formula (I) is optionally substituted heterocycloalkyl, such as, for example, pyrrolidinyl, tetrahydrofuranyl, tetrahydropyranyl, tetrahydrothiophenyl, isoxazolidinyl, oxazolidinyl, pyrazolidinyl, imidazolidinyl, or thiazolidinyl.

In some embodiments, R⁵ or R⁶ in the compounds of formula (I) is tetrahydropyran-4-yl.

In some aspects, R⁵ and R⁶ in the compounds of formula (I), together with the nitrogen atom to which they are both attached, form an optionally substituted 3-12-membered heterocycloalkyl ring, an optionally substituted 5-12-membered bridged heterocycloalkyl ring; an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, and 5-12-membered spiroheterocycloalkyl ring system may optionally include, in addition to the nitrogen atom to which both R⁵ and R⁶ are attached, 1-3 heteroatoms that are each independently O, S, or N.

In some aspects, R⁵ and R⁶ in the compounds of formula (I), together with the nitrogen atom to which they are both attached, form an optionally substituted 3-12-membered heterocycloalkyl ring. Non-limiting examples of such heterocycloalkyl rings include the following:

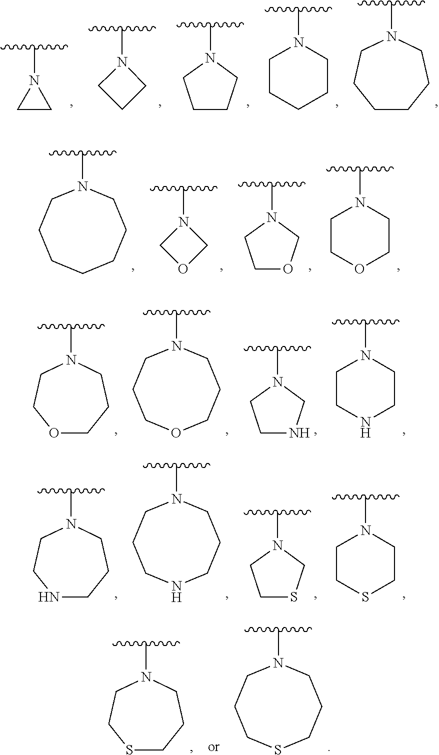

In some embodiments, R⁵ and R⁶, together with the nitrogen atom to which they are both attached, form a piperazinyl group,

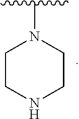 [SMILES: *N1CCNCC1]

In some embodiments, R⁵ and R⁶, together with the nitrogen atom to which they are both attached, form an oxazepanyl group,

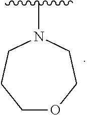 [SMILES: *N1CCCOCC1]

In some embodiments, R⁵ and R⁶, together with the nitrogen atom to which they are both attached, form a 1,1-dioxo-thiomorpholin-4-yl group,

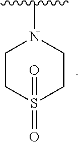

In some embodiments, the optionally substituted 3-12-membered heterocycloalkyl ring is substituted with at least one C₁-C₆alkyl group, such as, for example, methyl, ethyl, n-propyl, iso-propyl, n-butyl, t-butyl, iso-butyl, sec-butyl, n-pentyl, n-hexyl, and the like.

In some embodiments, the optionally substituted 3-12-membered heterocycloalkyl ring is substituted with at least one —CH₃ group.

In some embodiments, R⁵ and R⁶, together with the nitrogen atom to which they are both attached, form a 1-methylpiperazin-4-yl group,

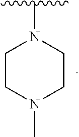 [SMILES: *N1CCN(C)CC1]

In some embodiments, R⁵ and R⁶, together with the nitrogen atom to which they are both attached, form a pyrrolidin-1-yl group,

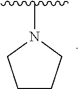 [SMILES: *N1CCCC1]

In some embodiments, R⁵ and R⁶, together with the nitrogen atom to which they are both attached, form a 2,2-dimethylpyrrolidin-1-yl group,

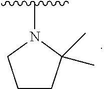 [SMILES: *N1CCCC1(C)C]

In other embodiments, R⁵ and R⁶, together with the nitrogen atom to which they are both attached, form a 3,3-dimethylpyrrolidin-1-yl group,

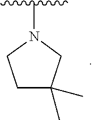 [SMILES: *N1CCC(C)(C)C1]

In other embodiments, R⁵ and R⁶, together with the nitrogen atom to which they are both attached, form a 3,3-dimethylazetidin-1-yl group,

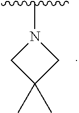 [SMILES: *N1CC(C)(C)C1]

In other embodiments, R⁵ and R⁶, together with the nitrogen atom to which they are both attached, form a 3-methoxyazetidin-1-yl group,

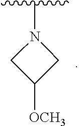 [SMILES: *N1CC(OC)C1]

In other embodiments R⁵ and R⁶, together with the nitrogen atom to which they are both attached, form a (R)-3-methoxyazetidin-1-yl group.

In other embodiments, R⁵ and R⁶, together with the nitrogen atom to which they are both attached, form a (S)-3-methoxyazetidin-1-yl group.

In other embodiments, R⁵ and R⁶, together with the nitrogen atom to which they are both attached, form a 2-methyl-pyrrolidin-1-yl group,

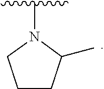 [SMILES: *N1CCCC1C]

In other embodiments R⁵ and R⁶, together with the nitrogen atom to which they are both attached, form a (R)-2-methyl-pyrrolidin-1-yl group.

In other embodiments, R⁵ and R⁶, together with the nitrogen atom to which they are both attached, form a (S)-2-methyl-pyrrolidin-1-yl group.

In other embodiments, R⁵ and R⁶, together with the nitrogen atom to which they are both attached, form a 2-methoxymethyl-pyrrolidin-1-yl group,

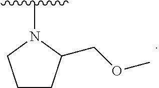 [SMILES: *N1CCCC1COC]

In other embodiments R⁵ and R⁶, together with the nitrogen atom to which they are both attached, form a (R)-2-methoxymethyl-pyrrolidin-1-yl group.

In other embodiments, R⁵ and R⁶, together with the nitrogen atom to which they are both attached, form a (S)-2-methoxymethyl-pyrrolidin-1-yl group.

In other embodiments, R⁵ and R⁶, together with the nitrogen atom to which they are both attached, form a 3-methyl-pyrrolidin-1-yl group,

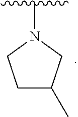 [SMILES: *N1CCC(C)C1]

In other embodiments R⁵ and R⁶, together with the nitrogen atom to which they are both attached, form a (R)-3-methyl-pyrrolidin-1-yl group.

In other embodiments, R⁵ and R⁶, together with the nitrogen atom to which they are both attached, form a (S)-3-methyl-pyrrolidin-1-yl group.

In other embodiments, R⁵ and R⁶, together with the nitrogen atom to which they are both attached, form a 3-methoxy-pyrrolidin-1-yl group,

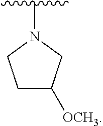 [SMILES: *N1CCC(OC)C1]

In other embodiments R⁵ and R⁶, together with the nitrogen atom to which they are both attached, form a (R)-3-methoxy-pyrrolidin-1-yl group.

In other embodiments, R⁵ and R⁶, together with the nitrogen atom to which they are both attached, form a (S)-3-methoxy-pyrrolidin-1-yl group.

In some embodiments, R⁵ and R⁶, together with the nitrogen atom to which they are both attached, form a 2-methyl-piperidin-1-yl group,

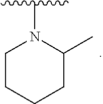 [SMILES: *N1CCCCC1C]

In some embodiments, R⁵ and R⁶, together with the nitrogen atom to which they are both attached, form a (R)-2-methyl-piperidin-1-yl group.

In other embodiments, R and R⁶, together with the nitrogen atom to which they are both attached, form a (S)-2-methyl-piperidin-1-yl group.

In some embodiments, R⁵ and R⁶, together with the nitrogen atom to which they are both attached, form a 2,6-dimethyl-piperidin-1-yl group,

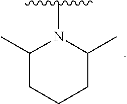

In some embodiments, R⁵ and R⁶, together with the nitrogen atom to which they are both attached, form a (R,R)-2,6-dimethyl-piperidin-1-yl group.

In some embodiments, R⁵ and R⁶, together with the nitrogen atom to which they are both attached, form a (R,S)-2,6-dimethyl-piperidin-1-yl group.

In some embodiments, R⁵ and R⁶, together with the nitrogen atom to which they are both attached, form a (S,R)-2,6-dimethyl-piperidin-1-yl group.

In some embodiments, R⁵ and R⁶, together with the nitrogen atom to which they are both attached, form a (S,S)-2,6-dimethyl-piperidin-1-yl group.

In some embodiments, R⁵ and R⁶, together with the nitrogen atom to which they are both attached, form a 3,5-dimethyl-morpholin-4-yl group,

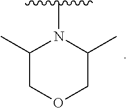

In some embodiments, R⁵ and R⁶, together with the nitrogen atom to which they are both attached, form a (R,R)-3,5-dimethyl-morpholin-4-yl group.

In some embodiments, R⁵ and R⁶, together with the nitrogen atom to which they are both attached, form a (R,S)-3,5-dimethyl-morpholin-4-yl group.

In some embodiments, R⁵ and R⁶, together with the nitrogen atom to which they are both attached, form a (S,R)-3,5-dimethyl-morpholin-4-yl group.

In some embodiments, R⁵ and R⁶, together with the nitrogen atom to which they are both attached, form a (S,S)-3,5-dimethyl-morpholin-4-yl group.

In some embodiments, R⁵ and R⁶, together with the nitrogen atom to which they are both attached, form a 2,6-dimethyl-morpholin-4-yl group,

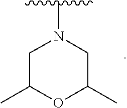

In some embodiments, R⁵ and R⁶, together with the nitrogen atom to which they are both attached, form a (R,R)-2,6-dimethyl-morpholin-4-yl group.

In some embodiments, R⁵ and R⁶, together with the nitrogen atom to which they are both attached, form a (R,S)-2,6-dimethyl-morpholin-4-yl group.

In some embodiments, R⁵ and R⁶, together with the nitrogen atom to which they are both attached, form a (S,R)-2,6-dimethyl-morpholin-4-yl group.

In some embodiments, R⁵ and R⁶, together with the nitrogen atom to which they are both attached, form a (S,S)-2,6-dimethyl-morpholin-4-yl group.

In some embodiments, R⁵ and R⁶, together with the nitrogen atom to which they are both attached, form a 3-ethyl-morpholin-4-yl group,

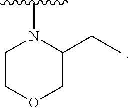 [SMILES: *N1CCOCC1CC]

In some embodiments, R⁵ and R⁶, together with the nitrogen atom to which they are both attached, form a (R)-3-ethyl-morpholin-4-yl group.

In other embodiments, R⁵ and R⁶, together with the nitrogen atom to which they are both attached, form a (S)-3-ethyl-morpholin-4-yl group.

In some embodiments, R⁵ and R⁶, together with the nitrogen atom to which they are both attached, form a 2-ethyl-morpholin-4-yl group,

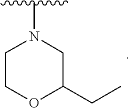 [SMILES: *N1CCOC(CC)C1]

In some embodiments, R⁵ and R⁶, together with the nitrogen atom to which they are both attached, form a (R)-2-ethyl-morpholin-4-yl group.

In other embodiments, R⁵ and R⁶, together with the nitrogen atom to which they are both attached, form a (S)-2-ethyl-morpholin-4-yl group.

In some embodiments, the optionally substituted 3-12-membered heterocycloalkyl ring is substituted with at least one C₁-C₆alkoxy group, such as, for example, methoxy, ethoxy, n-propoxy, iso-propoxy, n-butoxy, t-butoxy, iso-butoxy, sec-butoxy, n-pentoxy, n-hexoxy, and the like.

In some embodiments, R⁵ and R⁶, together with the nitrogen atom to which they are both attached, form a 4-methoxypiperidinyl group,

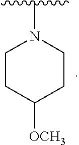 [SMILES: *N1CCC(OC)CC1]

In some embodiments, the optionally substituted 3-12-membered heterocycloalkyl ring is substituted with at least one halogen atom. In some embodiments, the halogen atom is —F.

In some embodiments, R⁵ and R⁶, together with the nitrogen atom to which they are both attached, form a 4,4-difluoropiperidin-1-yl group,

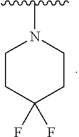 [SMILES: *N1CCC(F)(F)CC1]

In some embodiments, R⁵ and R⁶, together with the nitrogen atom to which they are both attached, form a 3,3-difluoropiperidin-1-yl group,

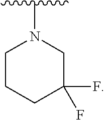 [SMILES: *N1CCCC(F)(F)C1]

In some embodiments, R⁵ and R⁶, together with the nitrogen atom to which they are both attached, form a 3,3-difluoroazetidin-1-yl group,

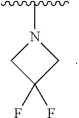 [SMILES: *N1CC(F)(F)C1]

In some embodiments, R⁵ and R⁶, together with the nitrogen atom to which they are both attached, form a 3,3-difluoropyrrolidin-1-yl group,

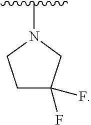 [SMILES: *N1CCC(F)(F)C1]

In some aspects, R⁵ and R⁶ in the compounds of formula (I), together with the nitrogen atom to which they are both attached, form an optionally substituted 5-12-membered bridged heterocycloalkyl ring. Non-limiting examples of such bridged heterocycloalkyl ring systems include:

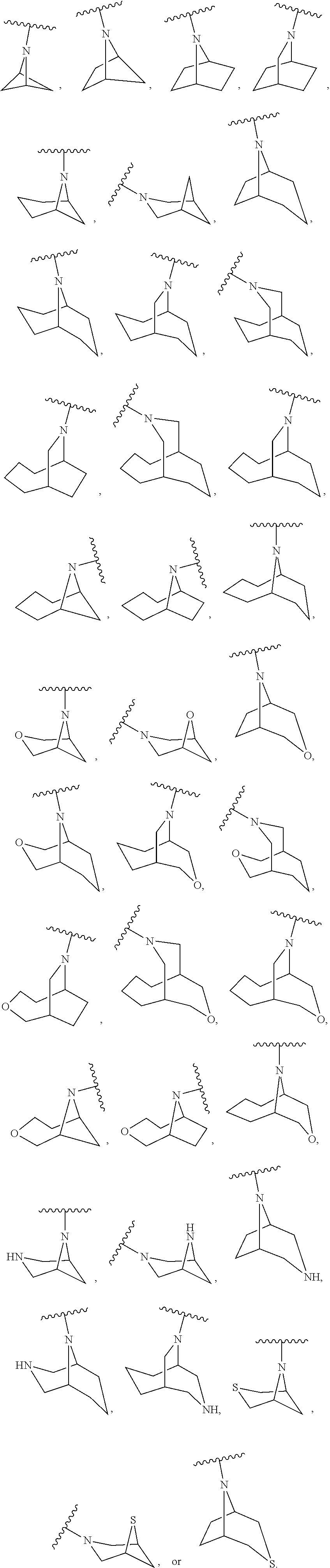

In some embodiments, R⁵ and R⁶, together with the nitrogen atom to which they are both attached, form a 2-azabicyclo[2.2.2]octan-2-yl group:

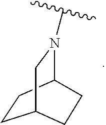 [SMILES: *N1CC2CCC1CC2]

In other embodiments, R⁵ and R⁶, together with the nitrogen atom to which they are both attached, form an 9-azabicyclo[3.3.1]nonan-9-yl group:

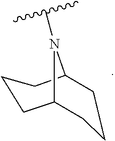 [SMILES: *N1C2CCCC1CCC2]

In other embodiments, R⁵ and R⁶, together with the nitrogen atom to which they are both attached, form an 3-azabicyclo[3.1.1]heptan-3-yl group,

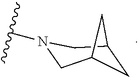 [SMILES: *N1CC2CC(C2)C1]

In other embodiments, R⁵ and R⁶, together with the nitrogen atom to which they are both attached, form a 7-azabicyclo[2.2.1]heptan-7-yl group,

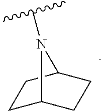 [SMILES: *N1C2CCC1CC2]

In some aspects, R⁵ and R⁶ in the compounds of formula (I), together with the nitrogen atom to which they are both attached, form an optionally substituted 5-12-membered spiroheterocycloalkyl ring. Non-limiting examples of such ring systems include:

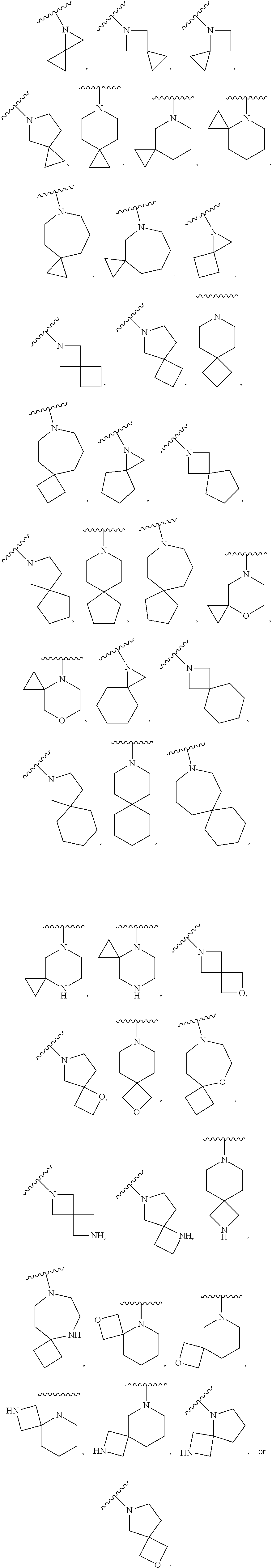

In some embodiments, R⁵ and R⁶, together with the nitrogen atom to which they are both attached, form a 1-azaspiro[3.3]heptan-1-yl group,

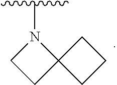 [SMILES: *N1CCC12CCC2]

In some embodiments, R⁵ and R⁶, together with the nitrogen atom to which they are both attached, form a 4-azaspiro[2.4]heptan-4-yl group,

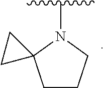 [SMILES: *N1CCCC12CC2]

In some embodiments, R⁵ and R⁶, together with the nitrogen atom to which they are both attached, form a 5-azaspiro[3.4]octan-5-yl group,

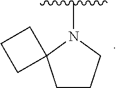 [SMILES: *N1CCCC12CCC2]

In some embodiments, R⁵ and R⁶, together with the nitrogen atom to which they are both attached, form a 6-azaspiro[3.4]octan-6-yl group,

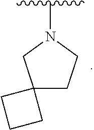 [SMILES: *N1CCC2(CCC2)C1]

In some embodiments, R⁵ and R⁶, together with the nitrogen atom to which they are both attached, form a 6-azaspiro[2.5]octan-6-yl group,

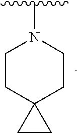 [SMILES: *N1CCC2(CC1)CC2]

In some embodiments, R⁵ and R⁶, together with the nitrogen atom to which they are both attached, form a 4-azaspiro[2.5]octan-4-yl group,

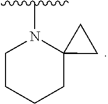 [SMILES: *N1CCCCC12CC2]

In some embodiments, R⁵ and R⁶, together with the nitrogen atom to which they are both attached, form a 7-azaspiro[3.5]nonan-7-yl group,

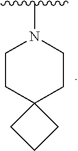 [SMILES: *N1CCC2(CCC2)CC1]

In some embodiments, R⁵ and R⁶, together with the nitrogen atom to which they are both attached, form a 7-oxa-4-azaspiro[2.5]octan-4-yl group,

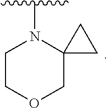 [SMILES: *N1CCOCC12CC2]

In some embodiments, R⁵ and R⁶, together with the nitrogen atom to which they are both attached, form a 4-oxa-7-azaspiro[2.5]octan-7-yl group,

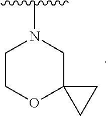 [SMILES: *N1CCOC2(CC2)C1]

In some embodiments, R⁵ and R⁶, together with the nitrogen atom to which they are both attached, form a 5-azaspiro[2.4]heptan-5-yl group,

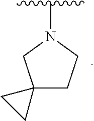 [SMILES: *N1CCC2(CC2)C1]

In some embodiments, R⁵ and R⁶, together with the nitrogen atom to which they are both attached, form a 2-oxa-5-azaspiro[3.5]nonan-5-yl group,

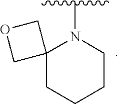 [SMILES: *N1CCCCC12COC2]

In some embodiments, R⁵ and R⁶, together with the nitrogen atom to which they are both attached, form a 2-oxa-6-azaspiro[3.5]nonan-6-yl group,

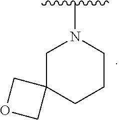 [SMILES: *N1CCCC2(COC2)C1]

In some embodiments, R⁵ and R⁶, together with the nitrogen atom to which they are both attached, form a 2-oxa-7-azaspiro[3.5]nonan-7-yl group,

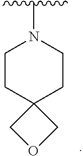 [SMILES: *N1CCC2(CC1)COC2]

In some embodiments, R⁵ and R⁶, together with the nitrogen atom to which they are both attached, form a 1-oxa-7-azaspiro[3.5]nonan-7-yl group,

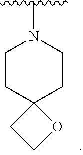 [SMILES: *N1CCC2(CCO2)CC1]

In some embodiments, R⁵ and R⁶, together with the nitrogen atom to which they are both attached, form a 7-azaspiro[4.4]nonan-7-yl group,

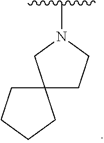 [SMILES: *N1CCC2(CCCC2)C1]

In some embodiments, R⁵ and R⁶, together with the nitrogen atom to which they are both attached, form a 2-oxa-5-azaspiro[3.4]octan-5-yl group,

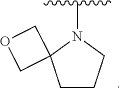 [SMILES: *N1CCCC12COC2]

In some embodiments, R⁵ and R⁶, together with the nitrogen atom to which they are both attached, form a 2-oxa-6-azaspiro[3.4]octan-6-yl group,

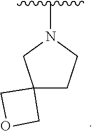 [SMILES: *N1CCC2(COC2)C1]

In some aspects, R⁵ and R⁶ in the compounds of formula (I), together with the nitrogen atom to which they are both attached, form an optionally substituted 4-12-membered fused heterocycloalkyl ring system. Non-limiting examples of such ring systems include:

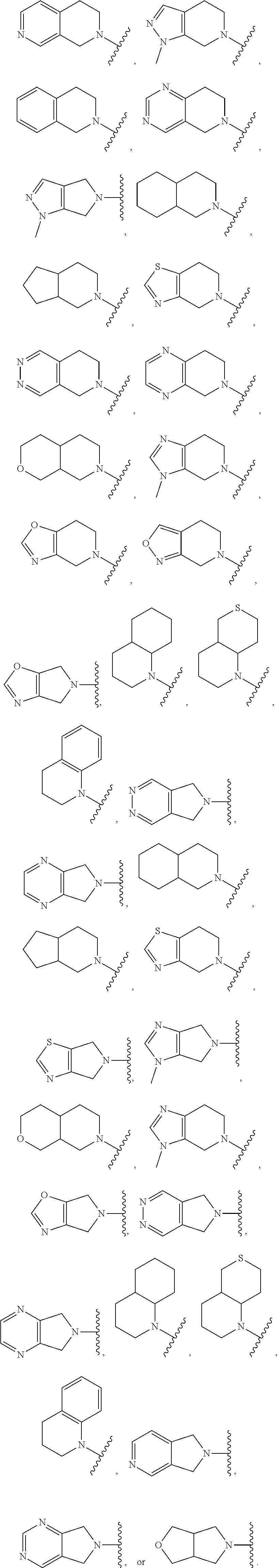

In some embodiments, R⁵ and R⁶, together with the nitrogen atom to which they are both attached, form a tetrahydro-1H,3H-furo[3,4-c]pyrrol-5-yl group:

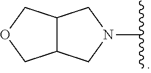 [SMILES: *N1CC2COCC2C1]

In some embodiments, R⁵ and R⁶, together with the nitrogen atom to which they are both attached, form a (R,R)-tetrahydro-1H,3H-furo[3,4-c]pyrrol-5-yl group.

In some embodiments, R⁵ and R⁶, together with the nitrogen atom to which they are both attached, form a (S,R)-tetrahydro-1H,3H-furo[3,4-c]pyrrol-5-yl group.

In some embodiments, R⁵ and R⁶, together with the nitrogen atom to which they are both attached, form a (R,S)-tetrahydro-1H,3H-furo[3,4-c]pyrrol-5-yl group.

In some embodiments, R⁵ and R⁶, together with the nitrogen atom to which they are both attached, form a (S,S)-tetrahydro-1H,3H-furo[3,4-c]pyrrol-5-yl group.

In some embodiments, R⁵ and R⁶, together with the nitrogen atom to which they are both attached, form a 3,4-dihydro-2,7-naphthyridin-2(1H)-yl group:

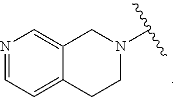 [SMILES: *N1CCc2ccncc2C1]

In some embodiments, R⁵ and R⁶, together with the nitrogen atom to which they are both attached, form a 1-methyl-4,5-dihydro-1H-pyrazolo[3,4-c]pyridin-6(7H)-yl group:

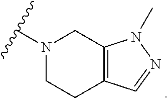 [SMILES: *N1CCc2cnn(C)c2C1]

In some aspects, the compounds of formula (I), are compounds of formula (IA) or formula (IB):

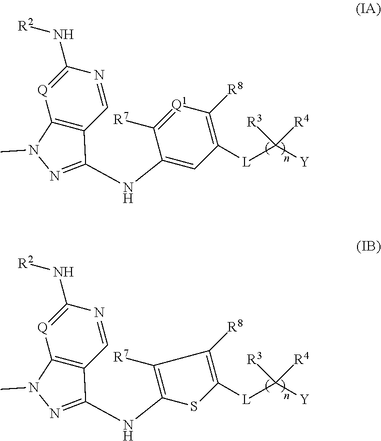

or pharmaceutically acceptable salts thereof, wherein Q¹ is N or CH; R⁷ is H, C₁-C₆alkyl, C₃-C₆cycloalkyl, halogen, —CN, or C₁-C₄fluoroalkyl; and R⁸ is H, C₁-C₆alkyl, C₃-C₆cycloalkyl, halogen, or C₁-C₄fluoroalkyl, and the other variables are as described above for formula (I).

In some embodiments of the compounds of formula (IA) or formula (IB), R⁷ is H, C₁-C₆alkyl, C₃-C₆cycloalkyl, halogen, —CN, or C₁-C₄fluoroalkyl.

In some embodiments of the compounds of formula (IA) or formula (IB), R⁷ is H.

In some embodiments of the compounds of formula (IA) or formula (IB), R⁷ is C₁-C₆alkyl, such as, for example, methyl, ethyl, n-propyl, iso-propyl, n-butyl, t-butyl, iso-butyl, sec-butyl, n-pentyl, n-hexyl, and the like.

In some embodiments of the compounds of formula (IA) or formula (IB), R⁷ is methyl.

In some embodiments of the compounds of formula (IA) or formula (IB), R⁷ is C₃-C₆cycloalkyl, such as, for example, cyclopropyl, cyclobutyl, cyclopentyl, cyclohexyl, and the like.

In some embodiments of the compounds of formula (IA) or formula (IB), R⁷ is halogen, such as, for example, —F, —Cl, —Br, or —I.

In some embodiments, R⁷ is —F.

In some embodiments of the compounds of formula (IA) or formula (IB), R⁷ is —CN.

In some embodiments of the compounds of formula (IA) or formula (IB), R⁷ is —C₁-C₄fluoroalkyl, such as, for example, C₄fluoroalkyl, C₃fluoroalkyl, C₂fluoroalkyl, C₁fluoroalkyl, and the like. In some embodiments, R⁷ is —CF₃.

In some embodiments of the compounds of formula (IA) or formula (IB), R⁸ is H, C₁-C₆alkyl, C₃-C₆cycloalkyl, halogen, or C₁-C₄fluoroalkyl.

In some embodiments of the compounds of formula (IA) or formula (IB), R₈ is H.

In some embodiments of the compounds of formula (IA) or formula (IB), R⁸ is C₁-C₆alkyl, such as, for example, methyl, ethyl, n-propyl, iso-propyl, n-butyl, t-butyl, iso-butyl, sec-butyl, n-pentyl, n-hexyl, and the like.

In some embodiments of the compounds of formula (IA) or formula (IB), R⁸ is methyl, i.e., —CH₃.

In some embodiments of the compounds of formula (IA) or formula (IB), R⁸ is C₃-C₆cycloalkyl, such as, for example, cyclopropyl, cyclobutyl, cyclopentyl, cyclohexyl, and the like.

In some embodiments of the compounds of formula (IA) or formula (IB), R⁸ is halogen, such as, for example, —F, —Cl, —Br, or —I.

In some embodiments, R⁸ is —F.

In some embodiments of the compounds of formula (IA) or formula (IB), R⁸ is —C₁-C₄fluoroalkyl, such as, for example, C₄fluoroalkyl, C₃fluoroalkyl, C₂fluoroalkyl, C₁fluoroalkyl and the like. In some embodiments, R⁸ is —CF₃.

In some embodiments, the compound of formula (I) is a compound of formula (IA).

In some embodiments of the compounds of formula (IA), Q¹ is N.

In other embodiments of the compounds of formula (IA), Q¹ is CH.

In some aspects, the compounds of formula (IA) are compounds of formula (IA-1):

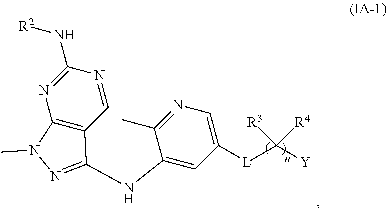

or pharmaceutically acceptable salts thereof, wherein the variables are as described above for the compounds of formula (I) and formula (IA).

In some aspects, the compounds of formula (IA) are compounds of formula (IA-1-1):

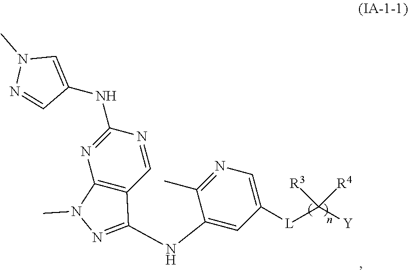

or pharmaceutically acceptable salts thereof, wherein the variables are as described above for the compounds of formula (I) and formula (IA-1).

In some aspects, the compounds of formula (IA) are compounds of formula (IA-1-2):

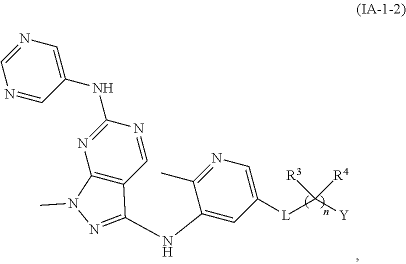

or pharmaceutically acceptable salts thereof, wherein the variables are as described above for the compounds of formula (I) and formula (IA-1).

In some aspects, the compounds of formula (IA) are compounds of formula (IA-1-3):

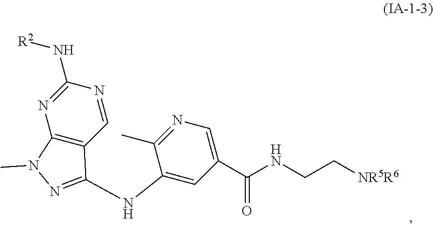

or pharmaceutically acceptable salts thereof, wherein the variables are as described above for the compounds of formula (I) and formula (IA-1).

In some aspects, the compounds of formula (IA) are compounds of formula (IA-2):

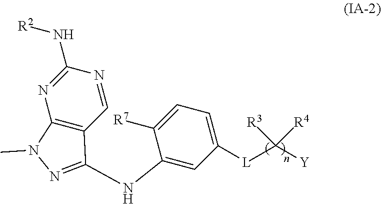

or pharmaceutically acceptable salts thereof, wherein the variables are as described above for the compounds of formula (I) and formula (IA).

In some embodiments of the compounds of formula (IA-2), R⁷ is —CH₃ or —F.

In some embodiments, the compounds of formula (IA-2) are compounds of formula (IA-2-1):

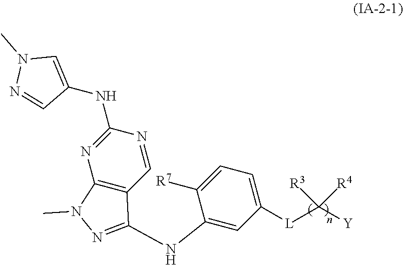

or pharmaceutically acceptable salts thereof, wherein the variables are as described above for the compounds of formula (I) and formula (IA-2).

In some embodiments, the compounds of formula (IA-2) are compounds of formula (IA-2-2):

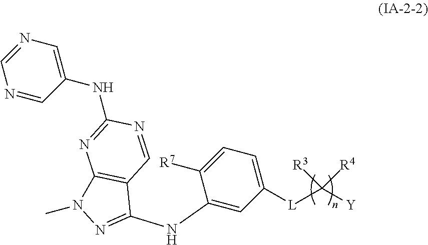

or pharmaceutically acceptable salts thereof, wherein the variables are as described above for the compounds of formula (I) and formula (IA-2).

In some embodiments, the compounds of formula (IA-2) are compounds of formula (IA-2-3):

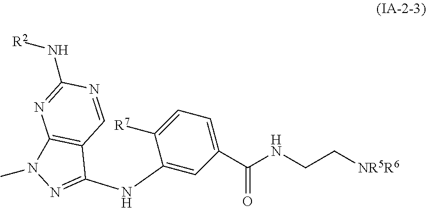

or pharmaceutically acceptable salts thereof, wherein the variables are as described above for the compounds of formula (I) and formula (IA-2).

In some embodiments, the compound of formula (I) is a compound of formula (IB).

In some aspects, the compounds of formula (IB) are compounds of formula IB-1:

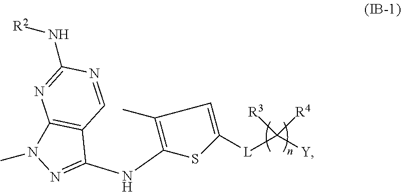

or pharmaceutically acceptable salts thereof, wherein the variables are as described above for the compounds of formula (I) and formula (IB).

In some embodiments, the compounds of formula (IB) are compounds of formula (IB-1-1):

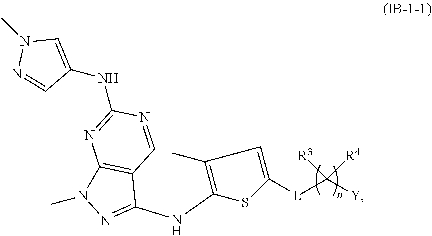

or pharmaceutically acceptable salts thereof, wherein the variables are as described above for the compounds of formula (I) and formula (IB-1).

In some embodiments, the compounds of formula (IB) are compounds of formula (IB-1-2):

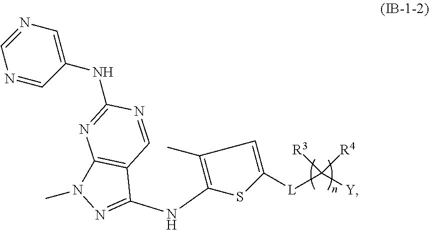

or pharmaceutically acceptable salts thereof, wherein the variables are as described above for the compounds of formula (I) and formula (IB-1).

In some embodiments, the compounds of formula (IB) are compounds of formula (IB-1-3):

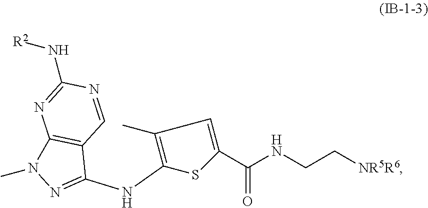

or pharmaceutically acceptable salts thereof, wherein the variables are as described above for the compounds of formula (I) and formula (IB-1).

In some aspects, the disclosure is directed to the compounds identified as Examples 1-122 below, or pharmaceutically acceptable salts thereof.

In some aspects, the disclosure is directed to the compounds identified as Examples 123-163 below, or pharmaceutically acceptable salts thereof.

References to formula (I) herein encompass any subgenera of those formula disclosed herein (e.g., formula (IA), (IA-1), (IA-1-1), (IA-1-2), (IA-1-3), (IA-2), (IA-2-1), (IA-2-2), (IA-2-3), (IB), (IB3-1), (IB-1-1), (IB3-1-2), (IB-1-3)).

Stereoisomers of compounds of formula (I) are also contemplated by the present disclosure. Thus, the disclosure encompasses all stereoisomers and constitutional isomers of any compound disclosed or claimed herein, including all enantiomers and diastereomers.

Pharmaceutically acceptable salts and solvates of the compounds of formula (I) are also within the scope of the disclosure.

Isotopic variants of the compounds of formula (I) are also contemplated by the present disclosure.

### Pharmaceutical Compositions and Methods of Administration

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. In some embodiments, the pharmaceutical compositions contain a compound of the present disclosure or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

In some embodiments, the compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day.

The exact dosage will depend upon the route of administration, the form in which the compound is administered, the subject to be treated, the body weight of the subject to be treated, and the preference and experience of the attending physician.

Unless otherwise noted, the amounts of the compounds described herein are set forth on a free base basis. That is, the amounts indicate that amount of the compound administered, exclusive of, for example, solvent (such as in solvates) or counterions (such as in pharmaceutically acceptable salts).

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion.

Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained.

Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10-oleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%), 100%, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical Compositions for Topical (e.g. Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

Compositions for inhalation may be delivered as a dry powder (either alone, as a mixture, for example a dry blend with lactose, or a mixed component particle, for example with phospholipids) from a dry powder inhaler or as an aerosol spray from a pressurised container, pump, spray, atomizer or nebuliser, with or without the use of a suitable propellant. Such devices are referred to in, for example, WO2013030802.

Where the inhalable form of the active ingredient is an aerosol composition, the inhalation device may be an aerosol vial provided with a valve adapted to deliver a metered dose, i.e. a metered dose inhaler. Where the inhalable form of the active ingredient is a nebulizable aqueous, organic or aqueous/organic dispersion, the inhalation device may be a nebulizer, such as an airjet nebulizer, or an ultrasonic nebulizer, or a hand-held nebulizer, sometimes referred to as a soft mist or soft spray inhaler, or a mechanical device which allows much smaller nebulized volumes than conventional nebulizers. Such devices are referred to in, for example, WO2013030802.

Where the inhalable form of the active ingredient is the finely divided particulate form, the inhalation device may be, for example, a dry powder inhalation device adapted to deliver dry powder from a capsule or blister containing a dry powder comprising a dosage unit or a multidose dry powder inhalation (MDPI) device adapted to deliver dry powder comprising a dosage unit upon actuation. The dry powder composition preferably contains a diluent or carrier, such as lactose, and a compound that helps to protect against product performance deterioration due to moisture e.g. magnesium stearate. Dry powder inhalation devices are referred to in, for example, WO2013030802

Thus, in some embodiments, the invention also includes (A) a compound of the invention, or a pharmaceutically acceptable salt thereof, in inhalable form; (B) an inhalable medicament comprising the compound in inhalable form together with a pharmaceutically acceptable carrier in inhalable form; (C) a pharmaceutical product comprising such a compound in inhalable form in association with an inhalation device; and (D) an inhalation device containing such a compound in inhalable form.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O; Knoben, James E; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

The disclosure also relates to methods of using the compounds described herein to treat in a subject in need thereof, a disease or disorder in which PDGFR signaling is implicated. These methods are accomplished by administering to the subject a compound of the disclosure in an amount effective to treat the disease or disorder.

In some aspects, the disclosure is directed to methods of using the compounds described herein to treat a disease or disorder in a subject in need thereof, wherein the disease or disorder is pulmonary hypertension (PH).

It should be understood that references herein to methods of treatment (e.g. methods of treating a disease or disorder in which PDGFR signaling is implicated in a subject in need thereof) using one or more compounds disclosed herein (e.g. a compound of formula (I)) should also be interpreted as references to: one or more compounds thereof (e.g. a compound of formula (I)) for use in methods of treatment (e.g., methods of treating a disease or disorder in which PDGFR signaling is implicated in a subject in need thereof); and/or the use of one or more compounds thereof (e.g. a compound of formula (I)) in the manufacture of a medicament for treating a pathological condition (e.g., a disease or disorder in which PDGFR signaling is implicated in a subject in need thereof).

Thus, in other aspects, the disclosure is directed to compounds of formula (I), or a pharmaceutically acceptable salt thereof, for use in treating a disease or disorder in which PDGFR signaling is implicated in a subject in need thereof. In some embodiments, the disease or disorder is pulmonary hypertension (PH). In other embodiments, the pulmonary hypertension is pulmonary arterial hypertension (PAH); PH secondary to heart failure; PH secondary to lung diseases and/or hypoxia; PH due to pulmonary artery obstruction; or PH due to unknown or rare diseases. In further embodiments, the pulmonary hypertension is pulmonary arterial hypertension (PAH).

In further aspects, the disclosure is directed to uses of compounds of formula (I), or a pharmaceutically acceptable salt thereof, in the manufacture of medicaments for treating a disease or disorder in which PDGFR signaling is implicated in a subject in need thereof. In some embodiments, the disease or disorder is pulmonary hypertension (PH). In other embodiments, the pulmonary hypertension is pulmonary arterial hypertension (PAH); PH secondary to heart failure; PH secondary to lung diseases and/or hypoxia; PH due to pulmonary artery obstruction; or PH due to unknown or rare diseases. In further embodiments, the pulmonary hypertension is pulmonary arterial hypertension (PAH).

In some embodiments, the pulmonary hypertension is pulmonary arterial hypertension (PAH) (WHO PH Group 1); PH secondary to heart failure (WHO PH Group 2); PH secondary to lung diseases and/or hypoxia (WHO PH Group 3); PH due to pulmonary artery obstruction (WHO Group 4); or PH due to unknown or rare diseases (WHO PH Group 5).

In some embodiments, the PAH (WHO PH Group 1) is idiopathic PAH, PAH with vasoreactivity, heritable PAH, drugs and toxins-induced PAH, PAH associated with connective tissue disease, PAH associated with HIV infection, PAH associated with portal hypertension, PAH associated with congenital heart disease, PAH associated with schistosomiasis, PAH in long-term responders to calcium channel blockers, PAH with overt signs of venous/capillaries involvement; persistent PH of the Newborn syndrome; or systemic sclerosis-associated PAH (SSc-PAH).

In some embodiments, the PAH secondary to heart failure (WHO PH Group 2) is PH due to heart failure with preserved ejection fraction, PH due to heart failure with reduced ejection fraction, valvular heart disease, or congenital post-capillary obstructive lesions.

In some embodiments, the PH secondary to lung diseases and/or hypoxia (WHO PH Group 3) is PH due to obstructive lung disease, PH due to restrictive lung disease, PH due to other lung diseases with mixed restrictive/obstructive pattern, PH due to hypoxia without lung disease, PH due to developmental lung disorders.

In some embodiments, the PH due to obstructive lung disease is PH due to chronic obstructive pulmonary disease (COPD).

In some embodiments, the PH due to restrictive lung disease is PH due to interstitial lung diseases (ILDs).

In some embodiments, the PH due to interstitial lung diseases (ILDs) is PH due to idiopathic pulmonary fibrosis (IPF).

In some embodiments, the PH due to pulmonary artery obstruction (WHO Group 4) is chronic thromboembolic PH (CTEPH) or PH due to other pulmonaty artery obstructions.

In some embodiments, the PH due to unknown or rare diseases (WHO PH Group 5) is PH due to hematologic disorders, PH due to systemic disorders, PH due to other disorders, or PH due to complex congenital heart disease.

In some aspects, the disclosure is directed to methods of using the compounds described herein to treat a disease or disorder in a subject in need thereof, wherein the disease or disorder is a respiratory disease.

In some embodiments, the respiratory disease is asthma.

In some aspects, the disclosure is directed to methods of using the compounds described herein to treat a disease or disorder in a subject in need thereof, wherein the disease or disorder is a fibrotic disease.

In some embodiments, the fibrotic disease is pulmonary fibrosis, cardiac fibrosis or liver fibrosis.

In some embodiments, the fibrotic disease is pulmonary fibrosis.

In some embodiments, the pulmonary fibrosis is an interstitial lung disease.

In some embodiments, the interstitial lung disease is idiopathic pulmonary fibrosis.

In some embodiments, the interstitial lung disease is rheumatoid arthritis-associated interstitial lung disease.

In some embodiments, the interstitial lung disease is systemic sclerosis-associated interstitial lung disease.

In some embodiments, the interstitial lung disease is connective tissue disease-associated interstitial lung disease.

In some embodiments, the interstitial lung disease is nonspecific interstitial pneumonia.

In some embodiments, the interstitial lung disease is unclassifiable interstitial lung disease.

In some embodiments, the interstitial lung disease is hypersensitivity pneumonitis.

In some embodiments, the interstitial lung disease is sarcoidosis.

In some embodiments, the interstitial lung disease is non-idiopathic pulmonary fibrosis interstitial lung disease.

In some aspects, the disclosure is directed to methods of using the compounds described herein to treat a disease or disorder in a subject in need thereof, wherein the disease or disorder is a dermatological disease.

In some embodiments, the dermatological disease or disorder is atopic dermatitis, scleroderma, or urticaria.

In some aspects, the disclosure is directed to methods of using the compounds described herein to treat a disease or disorder in a subject in need thereof, wherein the disease or disorder is an inflammatory disease or disorder.

In some embodiments, the inflammatory disease or disorder is allergic rhinitis, irritable bowel syndrome (IBS); or inflammatory bowel disease (IBD).

In some aspects, the disclosure is directed to methods of using the compounds described herein to treat a disease or disorder in a subject in need thereof, wherein the disease or disorder is an autoimmune disorder.

In some aspects, the disclosure is directed to methods of using the compounds described herein to treat a disease or disorder in a subject in need thereof, wherein the disease or disorder is a metabolic disease.

In some aspects, the disclosure is directed to methods of using the compounds described herein to treat a disease or disorder in a subject in need thereof, wherein the disease or disorder is vascular restenosis; age-related macular degeneration (AMD); irritable bowel syndrome (IBS); inflammatory bowel disease (IBD); obesity-cell related diseases; type I diabetes or type II diabetes.

In other embodiments, the disclosure is directed to methods of using the compounds described herein to treat a disease or disorder in a subject in need thereof, wherein the disease or disorder is pulmonary arterial hypertension (PAH).

In other embodiments, the disclosure is directed to methods of using the compounds described herein to treat a disease or disorder in a subject in need thereof, wherein the disease or disorder is PH secondary to heart failure (WHO PH Group 2).

In other embodiments, the disclosure is directed to methods of using the compounds described herein to treat a disease or disorder in a subject in need thereof, wherein the disease or disorder is PH due to heart failure with preserved ejection fraction.

In other embodiments, the disclosure is directed to methods of using the compounds described herein to treat a disease or disorder in a subject in need thereof, wherein the disease or disorder is PH due to heart failure with reduced ejection fraction.

In other embodiments, the disclosure is directed to methods of using the compounds described herein to treat a disease or disorder in a subject in need thereof, wherein the disease or disorder is valvular heart disease.

In other embodiments, the disclosure is directed to methods of using the compounds described herein to treat a disease or disorder in a subject in need thereof, wherein the disease or disorder is congenital post-capillary obstructive lesions.

In other embodiments, the disclosure is directed to methods of using the compounds described herein to treat a disease or disorder in a subject in need thereof, wherein the disease or disorder is PH secondary to lung diseases and/or hypoxia (WHO PH Group 3).

In other embodiments, the disclosure is directed to methods of using the compounds described herein to treat a disease or disorder in a subject in need thereof, wherein the disease or disorder is PH due to pulmonary artery obstruction (WHO Group 4).

In other embodiments, the disclosure is directed to methods of using the compounds described herein to treat a disease or disorder in a subject in need thereof, wherein the disease or disorder is chronic thromboembolic PH (CTEPH).

In other embodiments, the disclosure is directed to methods of using the compounds described herein to treat a disease or disorder in a subject in need thereof, wherein the disease or disorder is PH due to unknown or rare diseases (WHO PH Group 5).

In other embodiments, the disclosure is directed to methods of using the compounds described herein to treat a disease or disorder in a subject in need thereof, wherein the disease or disorder is idiopathic PAH.

In other embodiments, the disclosure is directed to methods of using the compounds described herein to treat a disease or disorder in a subject in need thereof, wherein the disease or disorder is PAH associated with connective tissue disease.

In other embodiments, the disclosure is directed to methods of using the compounds described herein to treat a disease or disorder in a subject in need thereof, wherein the disease or disorder is systemic sclerosis-associated PAH (SSc-PAH).

In other embodiments, the disclosure is directed to methods of using the compounds described herein to treat a disease or disorder in a subject in need thereof, wherein the disease or disorder is PH secondary to interstitial lung diseases (ILDs).

In other embodiments, the disclosure is directed to methods of using the compounds described herein to treat a disease or disorder in a subject in need thereof, wherein the disease or disorder is PH secondary to chronic obstructive pulmonary disease (COPD).

In other embodiments, the disclosure is directed to methods of using the compounds described herein to treat a disease or disorder in a subject in need thereof, wherein the disease or disorder is PH secondary to idiopathic pulmonary fibrosis (IPF).

In treatment methods according to the disclosure, an effective amount of a pharmaceutical agent according to the disclosure is administered to a subject suffering from or diagnosed as having such a disease or disorder. An “effective amount” means an amount or dose sufficient to generally bring about the desired therapeutic benefit in patients in need of such treatment for the designated disease or disorder. Effective amounts or doses of the compounds of the present disclosure may be ascertained by routine methods such as modeling, dose escalation studies or clinical trials, and by taking into consideration routine factors, e.g., the mode or route of administration or drug delivery, the pharmacokinetics of the compound, the severity and course of the disease or disorder, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

In addition, the compounds of the disclosure may be used in combination with additional active ingredients in the treatment of the above diseases or disorders. The additional active ingredients may be coadministered separately with a compound of the disclosure or included with such an agent in a pharmaceutical composition according to the disclosure. The combination may serve to increase efficacy (e.g., by including in the combination a compound potentiating the potency or effectiveness of an active agent according to the disclosure), decrease one or more side effects, or decrease the required dose of the active agent according to the disclosure.

### Aspects

Aspect 1: A compound of formula (I):

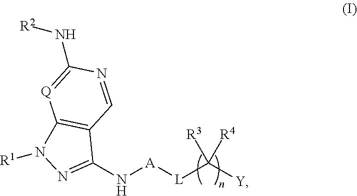

or a pharmaceutically acceptable salt thereof, wherein

A is an optionally substituted phenyl ring, an optionally substituted pyridinyl ring, or an optionally substituted 5-membered heteroaryl ring containing 1-2 heteroatoms that are each independently O, N, or S;

R¹ is an optionally substituted C₁-C₆alkyl group;

R² is optionally substituted aryl, optionally substituted heteroaryl, optionally substituted cycloalkyl, optionally substituted C₁-C₆-alkyl-cycloalkyl, or optionally substituted heterocycloalkyl;

Q is N or CH;

n is 1, 2, or 3;

R³ and R⁴ are each independently H, C₁-C₆alkyl, C₃-C₅cycloalkyl,
or, when n is 2 or 3, one R³ and one R⁴ attached to the same carbon atom, together with that carbon atom, may form a C₃-C₆cycloalkyl ring and one R³ and one

R⁴ attached to the same carbon atom, together with that carbon atom, may form a carbonyl group (>C═O);

L is —C(O)NH—, —NHC(O)—, or —NHC(O)NH, wherein when n is 1 and Y is —NR⁵R⁶, then L is —NHC(O)—;

Y is substituted or unsubstituted 3-7 membered cycloalkyl, optionally substituted 4-7 membered heterocycloalkyl, or —NR⁵R⁶;

R⁵ and R⁶ are each independently optionally substituted aryl, optionally substituted heteroaryl, optionally substituted alkyl, optionally substituted cycloalkyl, or optionally substituted heterocycloalkyl; or R⁵ and R⁶, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system optionally includes, in addition to the nitrogen atom to which both R⁵ and R⁶ are attached, 1-3 other heteroatoms that are each independently O, S, or N.

Aspect 2: The compound according to claim 1, wherein the compound of formula (I) is a compound of formula (IA) or formula (IB):

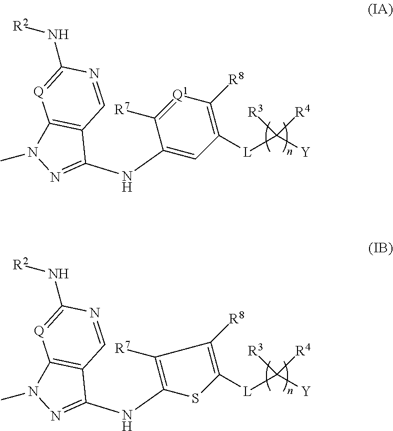

or a pharmaceutically acceptable salt thereof,

wherein
Q¹ is N or CH;
R⁷ is H, C₁-C₆alkyl, C₃-C₆cycloalkyl, halogen, —CN, or C₁-C₄fluoroalkyl; and
R⁸ is H, C₁-C₆alkyl, C₃-C₆cycloalkyl, halogen, or C₁-C₄fluoroalkyl.

Aspect 3: The compound according to Aspect 1 or Aspect 2, wherein Q is N.

Aspect 4: The compound according to Aspect 2 or Aspect 3, wherein said compound is a compound of formula (IA).

Aspect 5: The compound according to Aspect 4, wherein Q¹ is N.

Aspect 6: The compound according to Aspect 4, wherein Q¹ is CH.

Aspect 7: The compound according to any one of Aspects 2-6, wherein R⁷ is halogen or C₁-C₆alkyl.

Aspect 8: The compound according to any one of Aspects 2-7, wherein R⁸ is H.

Aspect 9: The compound according to Aspect 4, wherein the compound of formula IA is a compound of formula IA-1:

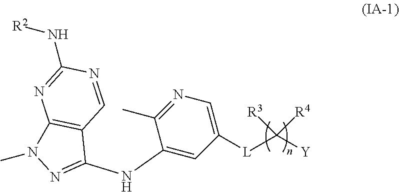

Aspect 10: The compound according to Aspect 4, wherein the compound of formula IA is a compound of formula IA-2:

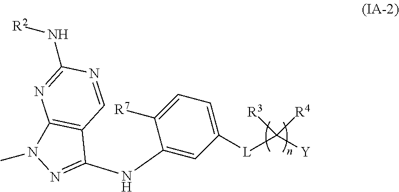

wherein R⁷ is —CH₃ or —F.

Aspect 11: The compound according to Aspect 2, wherein said compound is a compound of formula (IB).

Aspect 12: The compound according to Aspect 11, wherein the compound of formula IB is a compound of formula IB-1:

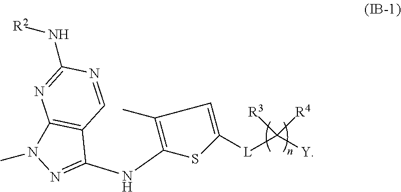

Aspect 13: The compound according to any one of Aspects 1-12, wherein R² is unsubstituted heteroaryl.

Aspect 14: The compound according to any one of Aspects 1-12, wherein R² is heterocycloalkyl.

Aspect 15: The compound according to any one of Aspects 1-12, wherein R² is substituted aryl.

Aspect 16: The compound according to any one of Aspects 1-12, wherein R² is substituted heteroaryl.

Aspect 17: The compound according to any one of the preceding Aspects, wherein L is —NHC(O)—.

Aspect 18: The compound according to any one of Aspects 1-16, wherein L is —C(O)NH—.

Aspect 19: The compound according to any one of Aspects 1-16, wherein L is —NHC(O)NH—.

Aspect 20: The compound according to any one of Aspects 1-19, wherein n=1.

Aspect 21: The compound according to any one of Aspects 1-19, wherein n=2.

Aspect 22: The compound according to any one of Aspects 1-19, wherein n=3.

Aspect 23: The compound according to any one of the preceding Aspects, wherein R³ and R⁴ are each H.

Aspect 24: The compound according to any one of the preceding Aspects, wherein an R³ and an R⁴ together with the carbon atom to which they are both attached, form a C₃-C₆cycloalkyl ring.

Aspect 25: The compound according to any one of the preceding Aspects, wherein an R³ and an R⁴ together with the carbon atom to which they are both attached, form a carbonyl group (>C═O).

Aspect 26: The compound according to any one of the preceding Aspects, wherein Y is a substituted or unsubstituted 3-7 membered cycloalkyl group.

Aspect 27: The compound according to any one of the preceding Aspects, wherein Y is —NR⁵R⁶.

Aspect 28: The compound according to Aspect 27, wherein R⁵ and R⁶ are each optionally substituted C₁-C₆alkyl.

Aspect 29: The compound according to Aspect 27, wherein R⁵ is optionally substituted C₁-C₆alkyl and R⁶ is optionally substituted cycloalkyl or optionally substituted heterocycloalkyl.

Aspect 30: The compound according to Aspect 27, wherein R⁵ and R⁶, together with the nitrogen atom to which they are both attached, form an optionally substituted 3-12-membered heterocycloalkyl ring.

Aspect 31: The compound according to Aspect 27, wherein R⁵ and R⁶, together with the nitrogen atom to which they are both attached, form an optionally substituted 5-12-membered bridged heterocycloalkyl ring.

Aspect 32: The compound according to Aspect 27, wherein R⁵ and R⁶, together with the nitrogen atom to which they are both attached, form an optionally substituted 5-12-membered spiroheterocycloalkyl ring.

Aspect 33: The compound according to Aspect 27, wherein R⁵ and R⁶, together with the nitrogen atom to which they are both attached, form an optionally substituted 4-12-membered fused heterocycloalkyl ring system.

Aspect 34: A pharmaceutical composition comprising a compound according to any one of Aspects 1-33, or a pharmaceutically acceptable salt thereof, and a pharmaceutically acceptable excipient.

Aspect 35: A method of treating a disease or disorder in which PDGFR signaling is implicated in a subject in need thereof, comprising administering to said subject an amount of a compound according to any one of Aspects 1-33, or a pharmaceutically acceptable salt thereof, that is effective to treat said disease or disorder.

Aspect 36: The method according to Aspect 35, wherein said disease or disorder is pulmonary hypertension (PH).

Aspect 37: The method according to Aspect 36, wherein said pulmonary hypertension is pulmonary arterial hypertension (PAH); PH secondary to heart failure; PH secondary to lung diseases and/or hypoxia; PH due to pulmonary artery obstruction; or PH due to unknown or rare diseases.

Aspect 38: The method according to Aspect 37, wherein said pulmonary hypertension is pulmonary arterial hypertension (PAH).

Compounds of Formula I in the present invention can be synthesized in accordance with general synthetic methods to those who are skilled in the art. The following reaction schemes are only meant to represent examples of the invention and are in no way meant to be a limit of the invention.

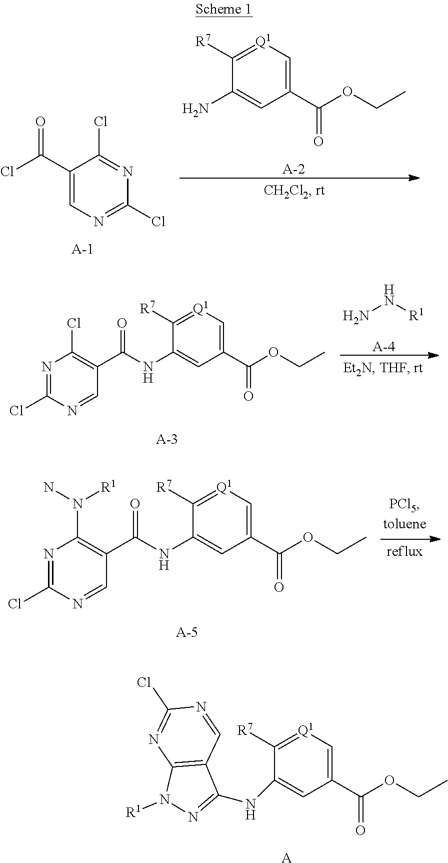

Scheme 1 show the synthesis of key intermediate A. 2,4-Dichloropyrimidine-5-carbonyl chloride (A-1) treated with compound A-2 in an appropriate solvent such as dichloromethane at room temperature to give compound A-3 which them treated with a hydrazine (A-4) in a solvent such as THF and a base such as triethylamine to yield compound A-5, compound A-5 refluxed with PCl₅ in toluene to produce key intermediate A.

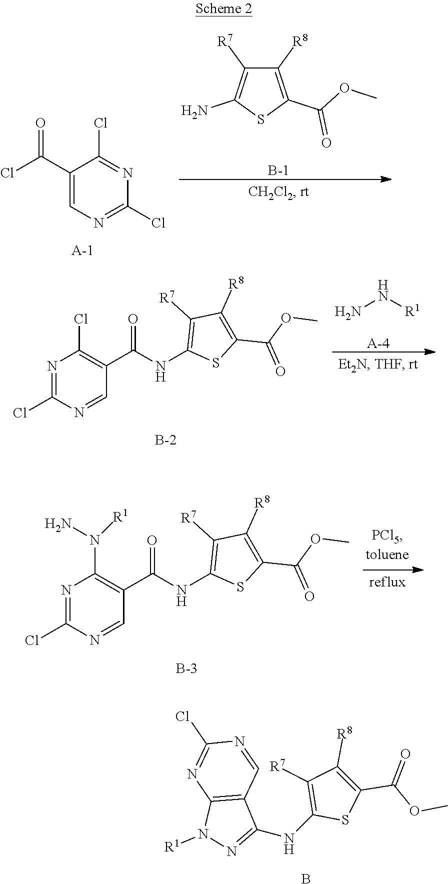

Scheme 2 show the synthesis of key intermediate B. 2,4-Dichloropyrimidine-5-carbonyl chloride (A-1) treated with aminothiophene ester B-1 in an appropriate solvent such as dichloromethane at room temperature to give compound B-2 which them treated with a hydrazine (A-4) in a solvent such as THF and a base such as triethylamine to yield compound B-3, compound B-3 then refluxed with PCl₅ in toluene to produce key intermediate B.

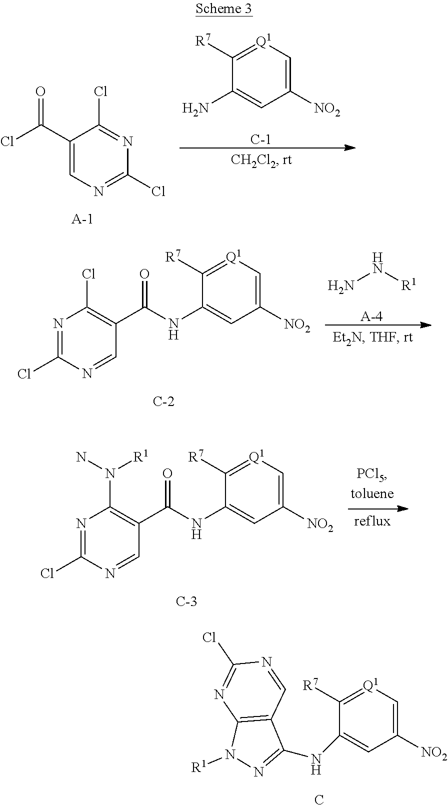

Scheme 3 illustrate the synthesis of key intermediate C. 2,4-Dichloropyrimidine-5-carbonyl chloride (A-1) treated with nitro compound C-1 in an appropriate solvent such as dichloromethane at room temperature to give compound C-2 which them treated with a hydrazine (A-4) in a solvent such as THF and a base such as triethylamine to yield compound C-3, compound C-3 then refluxed with PCl₅ in toluene to produce key intermediate C.

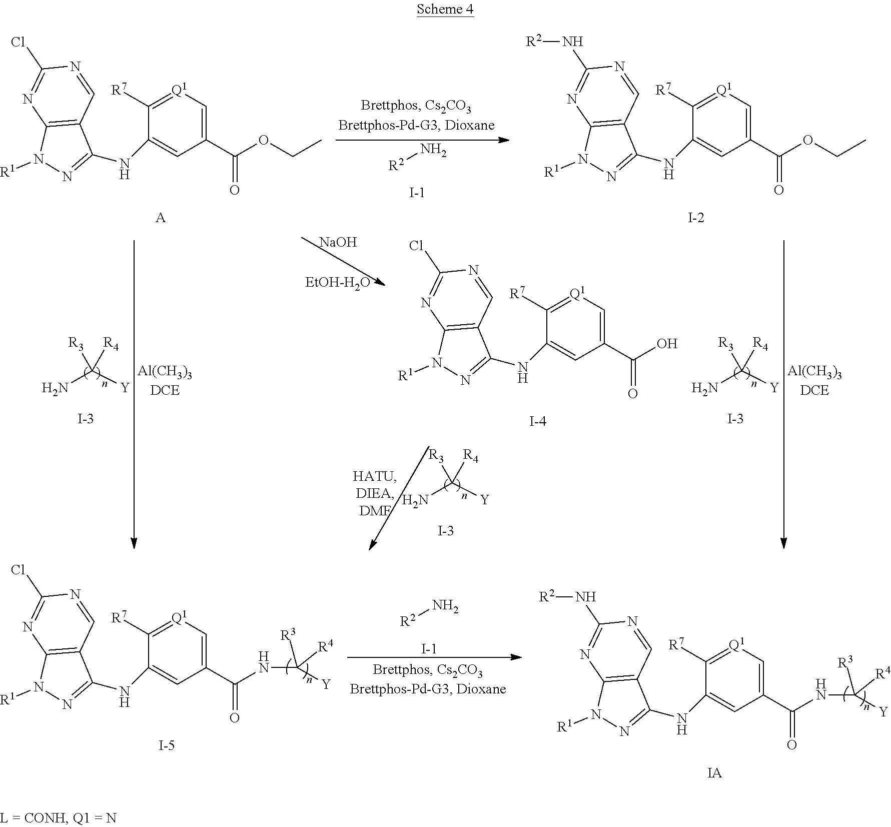

Scheme 4 show the synthesis of Formula IA while L=CONH, Q¹=N. Key intermediate A coupled with amine (I-1) in the present of a ligand such as Brettphos, a catalyst such as Brettphos-Pd-G3, a base such as Cs₂CO₃ in a solvent such as dioxane to yield compound I-2, compound I-2 then treated with an amine (I-3), Al(CH₃)₃ in dichloroethane to generate Formula IA. Alternatively key intermediate A first treated an amine (I-3), Al(CH₃)₃ in dichloroethane to give compound I-5, alternatively key intermediate A first hydrolyzed to acid I-4 in the present a base such as NaOH in a solvent such as ethanol-water followed by coupling with amine (I-3) in the present of HATU and DIEA in DMF to give compound I-5. Compound I-5 then coupled with amine (I-1) in the present of a ligand such as Brettphos, a catalyst such as Brettphos-Pd-G3, a base such as Cs₂CO₃ in a solvent such as dioxane to produce Formula IA.

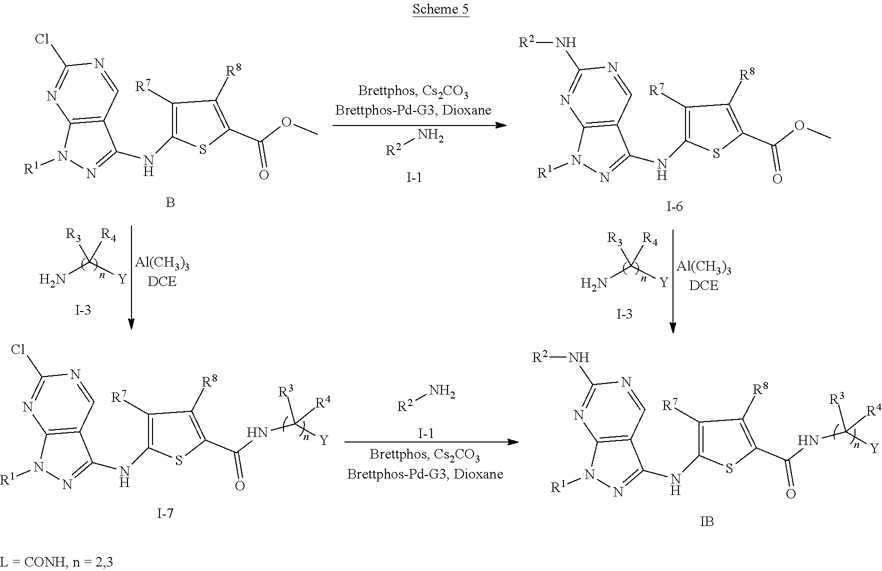

Scheme 5 show the synthesis of Formula IB while L=CONH. Key intermediate B coupled with amine (I-1) in the present of a ligand such as Brettphos, a catalyst such as Brettphos-Pd-G3, a base such as Cs₂CO₃ in a solvent such as dioxane to yield compound I-6, compound I-6 then treated with an amine (I-3), Al(CH₃)₃ in dichloroethane to generate Formula IB. Alternatively key intermediate B first treated an amine (I-3), Al(CH₃)₃ in dichloroethane to give compound I-7 which then coupled with amine (I-1) in the present of a ligand such as Brettphos, a catalyst such as Brettphos-Pd-G3, a base such as Cs₂CO₃ in a solvent such as dioxane to produce Formula IB.

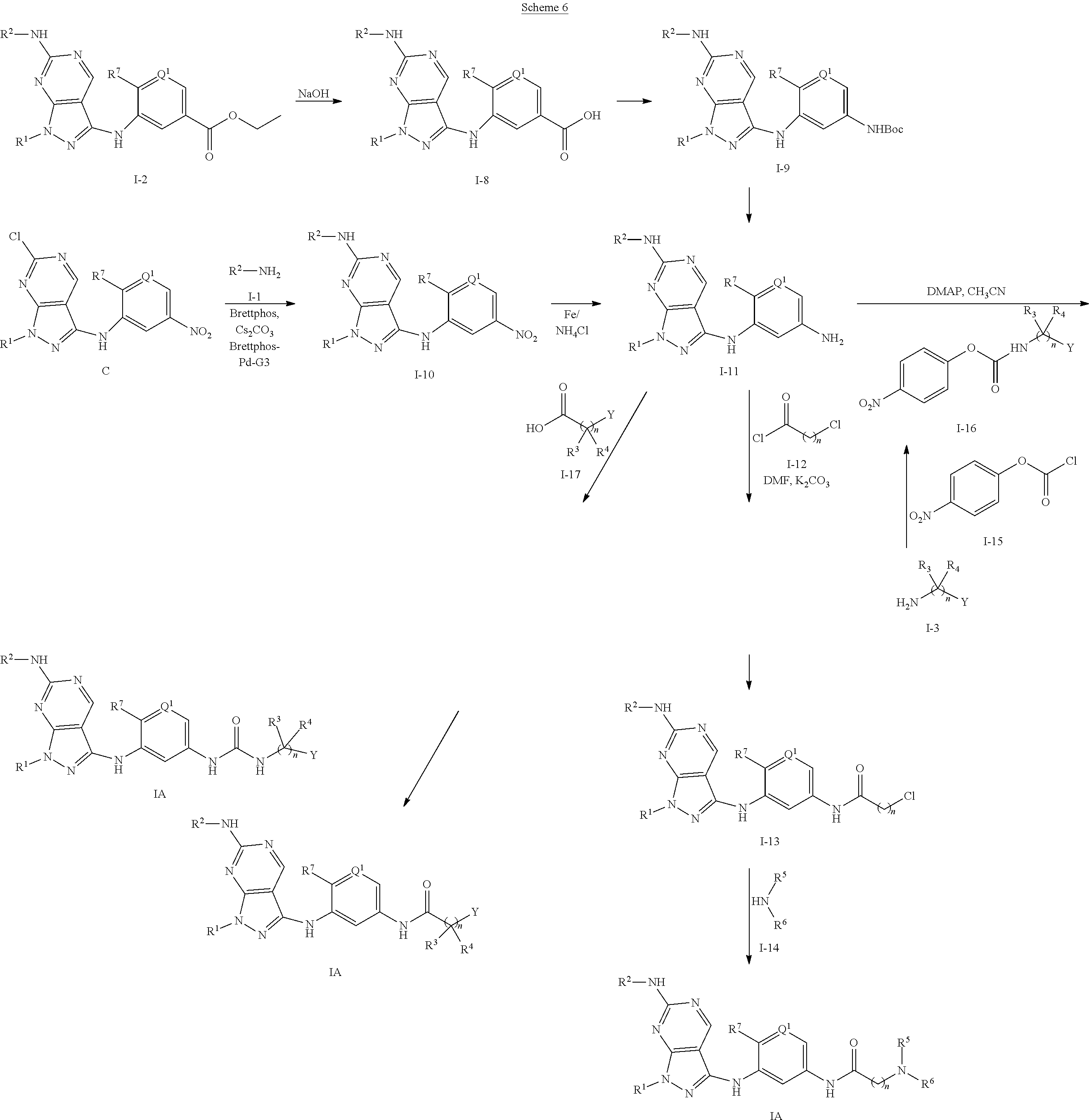

Compound I-2 first hydrolyzed to acid I-8 in the present of a base such as NaOH in a solvent such as ethanol-water, the acid I-8 then treated with DPPA in tert-butanol to yield a Boc protected compound I-9, de-Boc with HCl in dioxane to give an amine compound I-11. Alternatively, key intermediate C first reacted with an amine (I-1) in the present of a ligand such as Brettphos, a catalyst such as Brettphos-Pd-G3, a base such as Cs₂CO₃ in a solvent such as dioxane to yield compound I-10 which then reduced Fe/NH₄Cl to produce an amine compound I-11. Compound I-11 treated with chloroalkylacylchloride (I-12) in the present of a base such as K₂CO₃ in a solvent such as DMF to give compound I-13 which then reacted with amine I-14 in the present of a base such as K₂CO₃ in a solvent such as DMF to generate Formula IA. Alternatively, Compound I-11 coupled with acid (I-17) in the present of coupling reagent such HUTA, a base such as DIEA in a solvent such as DMF to afford Formula IA.

Amine (1-3) treated with 4-nitrophenylcarbamic chloride in a solvent such as dichloromethane to give a carbamate 1-16, the carbamate 1-16 then treated with compound I-11 in the present of DMAP in a solvent such as acetonitrile to yield Formula IA.

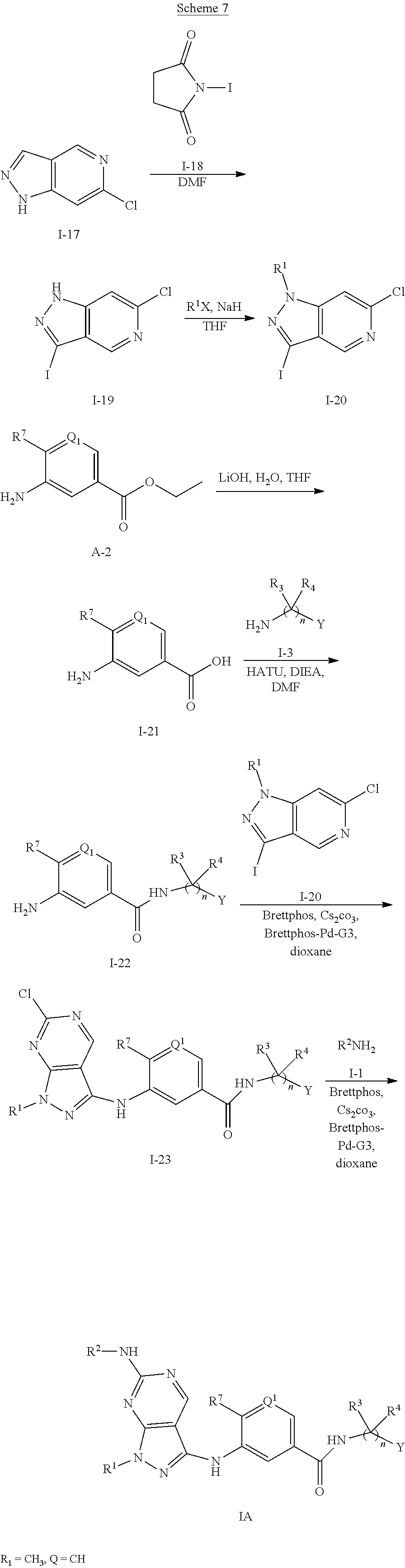

Scheme 7 illustrate the synthesis of Formula IA while Q=CH. 6-Chloro-1H-pyrazolo[4,3-c]pyridine (I-17) treated with 1-iodopyrrolidine-2,5-dione (I-18) in DMF to give compound I-19 which then reacted with alkyl halide in the present of base such as NaH in a solvent such as THF to generate compound 1-20.

The intermediate A-2 first hydrolyzed to an acid I-21, the acid I-21 then coupled with an amine 1-3 in the present of a coupling reagent such as HATU, a base such as DIEA in a solvent such as DMF to produce compound I-22, compound I-22 reacted with compound 1-20 in the present of a ligand such as Brettphos, a catalyst such as Brettphos-Pd-G3, a base such as Cs₂CO₃ in a solvent such as dioxane to yield compound 1-23. Compound I-23 then treated with an amine (I-1) in the present of a ligand such as Brettphos, a catalyst such as Brettphos-Pd-G3, a base such as Cs₂CO₃ in a solvent such as dioxane to yield Formula IA.

### EXAMPLES

### Example 1. N-(2-(4,4-difluoropiperidin-1-yl)ethyl)-6-methyl-5-((1-methyl-6-(pyrimidin-5-ylamino)-1H-pyrazolo[3,4-d]pyrimidin-3-yl)amino)nicotinamide

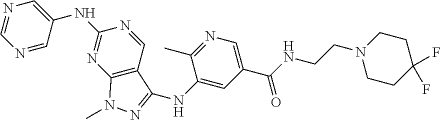 [SMILES: Cc1ncc(C(=O)NCCN2CCC(F)(F)CC2)cc1Nc1nn(C)c2nc(Nc3cncnc3)ncc12]

### Step a: 2-(4,4-difluoropiperidin-1-yl)acetonitrile

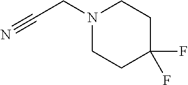 [SMILES: N#CCN1CCC(F)(F)CC1]

To a solution of 4, 4-difluoropiperidine (1.0 g, 6.35 mmol) and potassium carbonate (1.75 g, 12.69 mmol) in N,N-dimethylformamide (10 mL) was added 2-bromoacetonitrile (910 mg, 7.62 mmol) at room-temperature. The resulting mixture was stirred at 60° C. for 16 h before cooling to room temperature. The resulting mixture was quenched with water (10 mL) and extracted with ethyl acetate (10 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=7:3) to give the title compound 2-(4, 4-difluoropiperidin-1-yl)acetonitrile (700 mg, 68%) as a colorless oil.

### Step b: 2-(4, 4-difluoropiperidin-1-yl)ethanamine

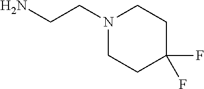 [SMILES: NCCN1CCC(F)(F)CC1]

To a solution of 2-(4,4-difluoropiperidin-1-yl)acetonitrile (700 mg, 4.37 mmol) in THF (4 mL) was added lithium aluminium hydride (180 mg, 4.81 mmol) by portions at 0° C. (ice/water). The mixture was stirred at 20° C. for 90 minutes before quenched with water (185 mg) at 0° C. The reaction mixture was filtered. The filtrate was concentrated to dryness under reduced pressure to afford the crude product 2-(4, 4-difluoropiperidin-1-yl)ethanamine as a colorless oil. ¹H NMR (400 MHz, METHANOL-d₄) δ 2.76 (t, J=6.4 Hz, 2H), 2.65-2.55 (m, 4H), 2.54-2.46 (m, 2H), 2.06-1.94 (m, 4H).

### Step c: Ethyl 5-(2,4-dichloropyrimidine-5-carboxamido)-6-methylnicotinate

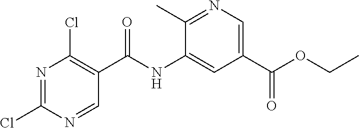 [SMILES: CCOC(=O)c1cnc(C)c(NC(=O)c2cnc(Cl)nc2Cl)c1]

To a solution of ethyl 5-amino-6-methylnicotinate (8.9 g, 49.4 mmol) in dichloromethane (450 mL) was added 2,4-dichloropyrimidine-5-carbonyl chloride (9.5 g, 44.9 mmol). The mixture stirred at 25° C. for 12 h and then concentrated under vacuum to give the crude product, which was purified by column chromatography over silica gel (eluent: dichloromethane/methyl alcohol=9:1) to give the title compound ethyl 5-(2,4-dichloropyrimidine-5-carboxamido)-6-methylnicotinate (16 g, 85%) as a yellow solid. LCMS (ESI): mass calcd. for C₂₁H₂₅BrN₆O₂S, 355.176; m/z found, 354.9 [M+H]⁺.

### Step d: Ethyl 5-(4-chloro-2-(2-methylhydrazinyl)pyrimidine-5-carboxamido)-6-methyl-nicotinate

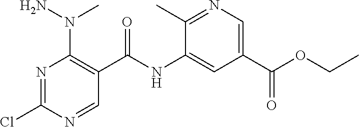 [SMILES: CCOC(=O)c1cnc(C)c(NC(=O)c2cnc(Cl)nc2N(C)N)c1]

To a solution of ethyl 5-(2,4-dichloropyrimidine-5-carboxamido)-6-methylnicotinate (10 g, 23.9 mmol) and TEA (8.4 mL, 59.9 mmol) in THF (100 mL) was added methylhydrazine (3.2 g, 27.5 mmol). The resulting mixture was quenched with water (300 mL) and extracted with ethyl acetate (300 mL×3). The combined organic phases were dried over anhydrous Na₂SO₄ and filtered. The filtrate was concentrated under reduced pressure to give the title compound ethyl 5-(4-chloro-2-(2-methylhydrazinyl)pyrimidine-5-carboxamido)-6-methylnicotinate (8.7 g, 99%) as a red solid. LCMS (ESI): mass calcd. for C₁₅H₁₇ClN₆O₃, 364.787; m/z found, 365.0 [M+H]⁺.

### Step e: ethyl 5-(2,4-dichloropyrimidine-5-carboxamido)-6-methylnicotinate

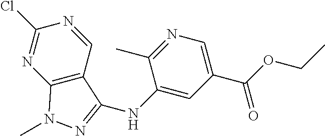 [SMILES: CCOC(=O)c1cnc(C)c(Nc2nn(C)c3nc(Cl)ncc23)c1]

To a solution of ethyl 5-(4-chloro-2-(2-methylhydrazinyl)pyrimidine-5-carboxamido)-6-methylnicotinate (8.7 g, 23.9 mmol) in toluene (150 mL) was added PCl₅ (4.9 g, 23.9 mmol). The mixture stirred at 120° C. for 12 h and then concentrated under vacuum to give the crude product, which was quenched with water (250 mg) and filtered. The filtrate was then concentrated to dryness under reduced pressure to give the title compound ethyl 5-(2,4-dichloropyrimidine-5-carboxamido)-6-methylnicotinate (6.9 g, 75%) as a yellow solid. LCMS (ESI): mass calcd. for C₁₅H₁₅ClN₆O₂, 346.772; m/z found, 347.0 [M+H]⁺.

### Step f: ethyl 6-methyl-5-((1-methyl-6-(pyrimidin-5-ylamino)-1H-pyrazolo[3,4-d]pyrimidin-3-yl)amino)nicotinate

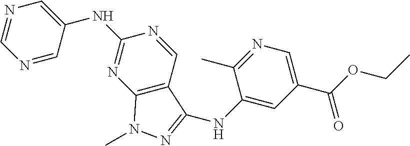 [SMILES: CCOC(=O)c1cnc(C)c(Nc2nn(C)c3nc(Nc4cncnc4)ncc23)c1]

To a solution of ethyl 5-(2,4-dichloropyrimidine-5-carboxamido)-6-methylnicotinate (1.9 g, 5.5 mmol), pyrimidin-5-amine (625 mg, 6.5 mmol) and Cs₂CO₃ (5.4 g, 16.4 mmol) in N,N-dimethylformamide (50 mL) was added Brettphos-Pd-G3 (993 mg, 1.1 mmol) and Brettphos (588 mg, 1.1 mmol). The mixture stirred at 120° C. for 12 h and then concentrated under vacuum to give the crude product, which was washed with MTBE (120 mL). The filtrate was then concentrated to dryness under reduced pressure to give the title compound ethyl 6-methyl-5-((1-methyl-6-(pyrimidin-5-ylamino)-1H-pyrazolo[3,4-d]pyrimidin-3-yl)amino)nicotinate (1.2 g, 47%) as a black solid. LCMS (ESI): mass calcd. for C₁₉H₁₉N₉O₂, 405.413; m/z found, 406.0 [M+H]⁺.

### Step g: 6-methyl-5-((1-methyl-6-(pyrimidin-5-ylamino)-1H-pyrazolo[3,4-d]pyrimidin-3-yl)amino)nicotinic acid

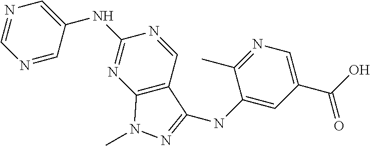 [SMILES: Cc1ncc(C(=O)O)cc1Nc1nn(C)c2nc(Nc3cncnc3)ncc12]

To a solution of ethyl 6-methyl-5-((1-methyl-6-(pyrimidin-5-ylamino)-1H-pyrazolo[3,4-d]pyrimidin-3-yl)amino)nicotinate (700 mg, 1.5 mmol) in methanol/THF/H₂O=1:3:1 (10 mL) was added lithium hydroxide (73 mg, 3.1 mmol) at room temperature. The reaction mixture was stirred at 20° C. for 2 h. The mixture was adjusted pH=3-4 with HCl (aq, 2 M). The mixture was filtered and washed with water (10 mL×3). The solid was evaporated under vacuum to give the desired product 6-methyl-5-((1-methyl-6-(pyrimidin-5-ylamino)-1H-pyrazolo[3,4-d]pyrimidin-3-yl)amino)nicotinic acid (550 mg, 85%) as a white solid. LCMS (ESI): mass calcd. for C₁₇H₁₅N₉O₂, 377.36; m/z found, 378.2 [M+H]⁺.

### Step h: N-(2-(4,4-difluoropiperidin-1-yl)ethyl)-6-methyl-5-((1-methyl-6-(pyrimidin-5-ylamino)-1H-pyrazolo[3,4-d]pyrimidin-3-yl)amino)nicotinamide

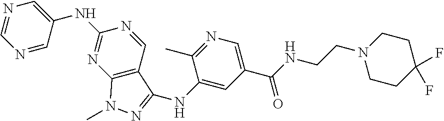 [SMILES: Cc1ncc(C(=O)NCCN2CCC(F)(F)CC2)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino) nicotinic acid (100 mg, 0.23 mmol), HATU (132 mg, 0.35 mmol) and N,N-diisopropylethylamine (90 mg, 0.70 mmol) in N,N-dimethylformamide (5 mL) was added 2-(4, 4-difluoropiperidin-1-yl)ethanamine (60 mg, 0.26 mmol). The mixture stirred at 50° C. for 2 h and then concentrated under vacuum to give the crude product, which was purified by preparative high-performance liquid chromatography over Column: Boston prime C18 150*30 mm*5 um to give the title compound N-(2-(4,4-difluoropiperidin-1-yl)ethyl)-6-methyl-5-((1-methyl-6-(pyrimidin-5-ylamino)-1H-pyrazolo[3,4-d]pyrimidin-3-yl)amino)nicotinamide (42 mg, 34%) as a yellow solid. LCMS (ESI): mass calcd. for C₂₄H₂₇F₂N₁₁O, 523.541; m/z found, 524.3 [M+H]⁺. ¹H NMR (400 MHz, METHANOL-d₄) δ 9.32 (s, 2H), 9.00 (s, 1H), 8.80-8.73 (m, 2H), 8.45 (d, J=1.8 Hz, 1H), 3.86 (s, 3H), 3.53 (t, J=6.6 Hz, 2H), 2.70-2.59 (m, 9H), 2.03-1.93 (m, 4H).

### Example 2. N-(2-(3,3-difluoropiperidin-1-yl)ethyl)-6-methyl-5-((1-methyl-6-(pyrimidin-5-ylamino)-1H-pyrazolo[3,4-d]pyrimidin-3-yl)amino)nicotinamide

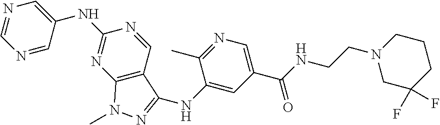 [SMILES: Cc1ncc(C(=O)NCCN2CCCC(F)(F)C2)cc1Nc1nn(C)c2nc(Nc3cncnc3)ncc12]

### Step a: 2-(3,3-difluoropiperidin-1-yl)acetonitrile

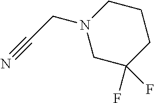 [SMILES: N#CCN1CCCC(F)(F)C1]

To a solution of 3,3-difluoropiperidine (400 mg, 2.5 mmol) and potassium carbonate (700 mg, 5.1 mmol) in N,N-dimethylformamide (6 mL) was added 2-bromoacetonitrile (365 mg, 3.0 mmol) at room-temperature. The resulting mixture was stirred at 60° C. for 12 h before cooling to room temperature. The resulting mixture was quenched with water (10 mL) and extracted with ethyl acetate (10 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=7:3) to give the title compound 2-(3,3-difluoropiperidin-1-yl)acetonitrile (250 mg, 61%) as a colorless oil.

### Step b: 2-(3,3-difluoropiperidin-1-yl)ethanamine

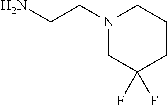 [SMILES: NCCN1CCCC(F)(F)C1]

To a solution of 2-(3,3-difluoropiperidin-1-yl)acetonitrile (250 mg, 1.6 mmol) in THF (4 mL) was added lithium aluminium hydride (65 mg, 1.7 mmol) by portions at 0° C. (ice/water). The resulting mixture was stirred at 20° C. for 90 minutes before quenched with water (65 mg) at 0° C. The reaction mixture was filtered. And the filtrate was concentrated to dryness under reduced pressure to afford the crude product 2-(4, 4-difluoropiperidin-1-yl)ethanamine as a colorless oil. ¹H NMR (400 MHz, METHANOL-d₄) δ 2.79-2.46 (m, 8H), 1.98-1.85 (m, 2H), 1.83-1.73 (m, 2H).

### Step c: N-(2-(3,3-difluoropiperidin-1-yl)ethyl)-6-methyl-5-((1-methyl-6-(pyrimidin-5-ylamino)-1H-pyrazolo[3,4-d]pyrimidin-3-yl)amino)nicotinamide

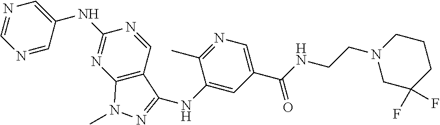 [SMILES: Cc1ncc(C(=O)NCCN2CCCC(F)(F)C2)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino) nicotinic acid (100 mg, 0.23 mmol), HATU (106 mg, 0.28 mmol) and N,N-diisopropylethylamine (90 mg, 0.70 mmol) in N,N-dimethylformamide (5 mL) was added 2-(3,3-difluoropiperidin-1-yl)ethanamine (60 mg, 0.26 mmol). The mixture stirred at 20° C. for 2 h and then concentrated under vacuum to give the crude product, which was purified by preparative high-performance liquid chromatography over Column: Phenomenex Gemini-NX C18 75*30 mm*3 um to give the title compound N-(2-(3,3-difluoropiperidin-1-yl)ethyl)-6-methyl-5-((1-methyl-6-(pyrimidin-5-ylamino)-1H-pyrazolo[3,4-d]pyrimidin-3-yl)amino)nicotinamide (31 mg, 25%) as a yellow solid. LCMS (ESI): mass calcd. for C₂₄H₂₇F₂N₁₁O, 523.541; m/z found, 524.3 [M+H]⁺. ¹H NMR (400 MHz, METHANOL-d₄) δ 9.33 (s, 2H), 9.01 (s, 1H), 8.78 (s, 2H), 8.45 (d, J=1.8 Hz, 1H), 3.88 (s, 3H), 3.55 (t, J=6.7 Hz, 2H), 2.76-2.65 (m, 7H), 2.56 (br d, J=4.6 Hz, 2H), 1.88 (dt, J=6.6, 13.3 Hz, 2H), 1.77 (br d, J=4.6 Hz, 2H).

### Example 3. (S)-6-methyl-5-((1-methyl-6-(pyrimidin-5-ylamino)-1H-pyrazolo [3,4-d]pyrimidin-3-yl)amino)-N-(2-(2-methylpyrrolidin-1-yl)ethyl)nicotinamide

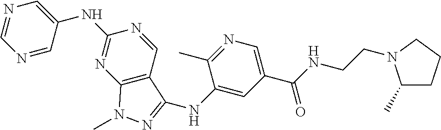 [SMILES: Cc1ncc(C(=O)NCCN2CCC[C@@H]2C)cc1Nc1nn(C)c2nc(Nc3cncnc3)ncc12]

### Step a: (S)-2-(2-methylpyrrolidin-1-yl)acetonitrile

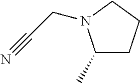

To a solution of (S)-2-methylpyrrolidine (1 g, 11.7 mmol) and potassium carbonate (3.7 g, 27 mmol) in acetonitrile (10 mL) was added 2-bromoacetonitrile (1.6 g, 12.9 mmol) at room temperature. The resulting mixture was stirred at 50° C. for 12 h before cooling to room-temperature. The resulting mixture was quenched with water (30 mL) and extracted with ethyl acetate (50 mL×3). The combined organic phases were dried over anhydrous Na₂SO₄ and filtered. The filtrate was concentrated under reduced pressure to give crude product, which was purified by column chromatography over silica gel (eluent: petroleum ether:ethyl acetate=3:1) to give the title compound (S)-2-(2-methylpyrrolidin-1-yl)acetonitrile (850 mg, 58%) as a pale yellow oil. ¹H NMR (400 MHz, CDCl₃) δ 3.64-3.56 (m, 2H), 2.96 (dt, J=3.0, 8.5 Hz, 1H), 2.59-2.47 (m, 2H), 2.00-1.88 (m, 1H), 1.83-1.64 (m, 2H), 1.39 (m, 1H), 1.03 (d, J=6.1 Hz, 3H).

### Step b: (S)-2-(2-methylpyrrolidin-1-yl)ethanamine

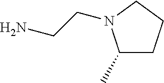

To a solution of (S)-2-methylpyrrolidine (500 mg, 4.0 mmol) in methanol (50 mL) was hydrogenated at room temperature with Raney Ni (24 mg, 0.40 mmol) as a catalyst in the presence of H₂ (15 psi) for 2 hours. After uptake of H₂ (3 eq), the catalyst filtered off and the filtrate was evaporated give the title compound (S)-2-(2-methylpyrrolidin-1-yl)ethanamine (270 mg, 52%) as pale yellow oil. ¹H NMR (400 MHz, CDCl₃) δ 3.18-3.08 (m, 1H), 3.05-2.90 (m, 1H), 2.88-2.66 (m, 2H), 2.28 (br d, J=5.7 Hz, 1H), 2.20-2.00 (m, 2H), 1.93-1.82 (m, 1H), 1.71-1.61 (m, 2H), 1.45-1.32 (m, 1H), 1.10-1.05 (m, 3H).

### Step c: (S)-6-methyl-5-((1-methyl-6-(pyrimidin-5-ylamino)-1H-pyrazolo[3,4-d]pyrimidin-3-yl)amino)-N-(2-(2-methylpyrrolidin-1-yl)ethyl)nicotinamide

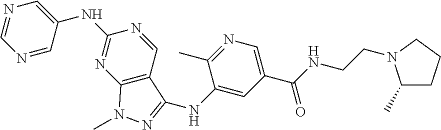 [SMILES: Cc1ncc(C(=O)NCCN2CCC[C@@H]2C)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino) nicotinic acid (140 mg, 0.36 mmol), HATU (277 mg, 0.73 mmol) and N,N-diisopropylethylamine (235 mg, 1.8 mmol) in DMF (5 mL) was added (S)-2-(2-methylpyrrolidin-1-yl)ethanamine (93 mg, 0.73 mmol). The resulting mixture was stirred at room temperature for 1 hour and then concentrated under vacuum to give the crude product, which was purified by preparative high-performance liquid chromatography over Column: Phenomenex Gemini-NX 80*40 mm*3 um to give the title compound (S)-6-methyl-5-((1-methyl-6-(pyrimidin-5-ylamino)-1H-pyrazolo[3,4-d]pyrimidin-3-yl)amino)-N-(2-(2-methylpyrrolidin-1-yl)ethyl)nicotinamide (26 mg, 14%) as a pale brown solid. LCMS (ESI): mass calcd. for C₂₄H₂₉N₁₁O, 487.2; m/z found, 488.5 [M+H]⁺. ¹H NMR (400 MHz, METHANOL-d₄) δ 9.36 (s, 2H), 9.03 (s, 1H), 8.81 (br d, J=6.9 Hz, 2H), 8.50 (s, 1H), 3.90 (s, 3H), 3.65 (br d, J=7.6 Hz, 1H), 3.55-3.47 (m, 1H), 3.16-3.00 (m, 2H), 2.68 (s, 3H), 2.45 (br d, J=6.9 Hz, 1H), 2.31 (td, J=9.1, 18.1 Hz, 2H), 2.00 (br s, 1H), 1.80 (br d, J=7.3 Hz, 2H), 1.44 (br s, 1H), 1.16 (br d, J=6.1 Hz, 3H).

### Example 4. N-(2-(5-azaspiro[3.4]octan-5-yl)ethyl)-6-methyl-5-((1-methyl-6-(pyrimidin-5-ylamino)-1H-pyrazolo[3,4-d]pyrimidin-3-yl)amino)nicotinamide

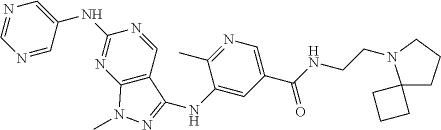 [SMILES: Cc1ncc(C(=O)NCCN2CCCC23CCC3)cc1Nc1nn(C)c2nc(Nc3cncnc3)ncc12]

### Step a:2-(5-azaspiro[3.4]octan-5-yl)acetonitrile

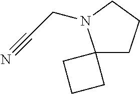 [SMILES: N#CCN1CCCC12CCC2]

To a solution of 5-azaspiro[3.4]octane (400 mg, 1.3 mmol) and potassium carbonate (884 mg, 6.4 mmol) in N,N-dimethylformamide (8 mL) was added 2-bromoacetonitrile (307 mg, 2.5 mmol) at room-temperature. The resulting mixture was stirred at 50° C. for 12 h before cooling to room temperature. The resulting mixture was quenched with water (10 mL) and extracted with ethyl acetate (10 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=4:1) to give the title compound 2-(5-azaspiro[3.4]octan-5-yl)acetonitrile (205 mg, 53%) as a colorless oil.

### Step b: 2-(5-azaspiro[3.4]octan-5-yl)ethanamine

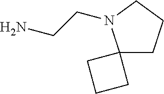 [SMILES: NCCN1CCCC12CCC2]

To a solution of 2-(5-azaspiro[3.4]octan-5-yl)acetonitrile (205 mg, 1.4 mmol) in THF (8 mL) was added lithium aluminium hydride (93 mg, 2.5 mmol) by portions at 0° C. (ice/water). The mixture was stirred at 20° C. for 90 minutes before quenched with water (100 mg) at 0° C. The reaction mixture was filtered. And the filtrate was concentrated to dryness under reduced pressure to afford the crude product 2-(5-azaspiro[3.4]octan-5-yl)ethanamine as a colorless oil. ¹H NMR (400 MHz, METHANOL-d₄) δ 2.65-2.58 (m, 2H), 2.51-2.45 (m, 2H), 2.44-2.39 (m, 2H), 2.04-1.95 (m, 2H), 1.77-1.72 (m, 2H), 1.60-1.53 (m, 2H), 1.52-1.45 (m, 4H).

### Step c: N-(2-(5-azaspiro[3.4]octan-5-yl)ethyl)-6-methyl-5-((1-methyl-6-(pyrimidin-5-ylamino)-1H-pyrazolo[3,4-d]pyrimidin-3-yl)amino)nicotinamide

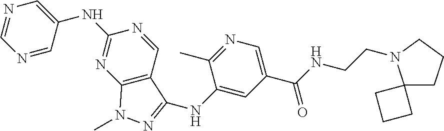 [SMILES: Cc1ncc(C(=O)NCCN2CCCC23CCC3)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino)nicotinic acid (100 mg, 0.25 mmol), HATU (114 mg, 0.3 mmol) and N,N-diisopropylethylamine (97 mg, 0.75 mmol) in N,N-dimethylformamide (5 mL) was added 2-(5-azaspiro[3.4]octan-5-yl)ethanamine (62 mg, 0.27 mmol). The mixture stirred at 50° C. for 2 h and then concentrated under vacuum to give the crude product, which was purified by preparative high-performance liquid chromatography over Column: Welch Xtimate C18 150*25 mm*5 um to give the title compound N-(2-(5-azaspiro[3.4]octan-5-yl)ethyl)-6-methyl-5-((1-methyl-6-(pyrimidin-5-ylamino)-1H-pyrazolo[3,4-d]pyrimidin-3-yl)amino)nicotinamide (31.0 mg, 23%) as a yellow solid. LCMS (ESI): mass calcd. for C₂₆H₃₁N₁₁O, 513.597; m/z found, 514.3 [M+H]⁺. ¹H NMR (400 MHz, METHANOL-d₄) δ 9.30 (s, 2H), 9.00 (s, 1H), 8.86 (d, J=1.5 Hz, 1H), 8.76 (s, 1H), 8.48 (s, 1H), 3.87 (s, 3H), 3.74 (br t, J=6.3 Hz, 2H), 3.37 (br t, J=7.5 Hz, 2H), 3.25 (br t, J=6.3 Hz, 2H), 2.66 (s, 3H), 2.55-2.46 (m, 2H), 2.25-2.17 (m, 2H), 2.09-1.95 (m, 4H), 1.92-1.84 (m, 2H).

### Example 5. (S)-6-methyl-5-((1-methyl-6-(pyrimidin-5-ylamino)-1H-pyrazolo[3,4-d]pyrimidin-3-yl)amino)-N-(2-(2-methylpiperidin-1-yl)ethyl)nicotinamide

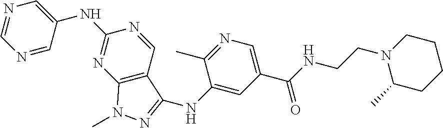 [SMILES: Cc1ncc(C(=O)NCCN2CCCC[C@@H]2C)cc1Nc1nn(C)c2nc(Nc3cncnc3)ncc12]

### Step a: (S)-tert-butyl (2-(2-methylpiperidin-1-yl)ethyl)carbamate

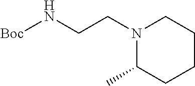 [SMILES: C[C@H]1CCCCN1CCNC(=O)OC(C)(C)C]

To a solution of (S)-2-methylpiperidine (300 mg, 3.0 mmol) and potassium carbonate (1 g, 7.5 mmol) in acetonitrile (15 mL) was added tert-butyl (2-bromoethyl)carbamate (0.75 g, 3.3 mmol) at room-temperature. The resulting mixture was stirred at 80° C. for 12 h before cooling to room temperature. The reaction was concentrated under reduced pressure to give crude product, which was purified by column chromatography over silica gel (petroleum ether/ethyl acetate=1:1) to give the title compound (S)-tert-butyl (2-(2-methylpiperidin-1-yl)ethyl)carbamate (380 mg, 52%) as a yellow solid. LCMS (ESI): mass calcd. for C₁₃H₂₆N₂O₂, 242.358; m/z found, 243.2 [M+H]⁺.

### Step b: (S)-2-(2-methylpiperidin-1-yl)ethanamine

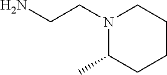

To a solution of (S)-tert-butyl (2-(2-methylpiperidin-1-yl)ethyl)carbamate (350 mg, 1.4 mmol) in DCM (2 mL) was added HCl/MeOH (2 mL, 4M) at 0° C. The resulting mixture was stirred at 20° C. for 1.5 hours. Then the reaction mixture was concentrated under reduced pressure to afford the crude product (S)-2-(2-methylpiperidin-1-yl)ethanamine as a HCl salt. LCMS (ESI): mass calcd. for C₈H₁₈N₂, 142.242; m/z found, 143.1 [M+H]⁺.

### Step c: N-(2-(5-azaspiro[3.4]octan-5-yl)ethyl)-6-methyl-5-((1-methyl-6-(pyrimidin-5-ylamino)-1H-pyrazolo[3,4-d]pyrimidin-3-yl)amino)nicotinamide

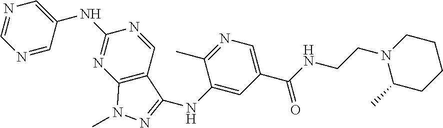 [SMILES: Cc1ncc(C(=O)NCCN2CCCC[C@@H]2C)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino)nicotinic acid (130 mg, 0.34 mmol), HATU (196 mg, 0.5 mmol) and N,N-diisopropylethylamine (133 mg, 1.0 mmol) in N,N-dimethylformamide (5 mL) was added (S)-2-(2-methylpiperidin-1-yl)ethanamine (137 mg, 0.41 mmol). The mixture stirred at 20° C. for 2 h and then concentrated under vacuum to give the crude product, which was purified by preparative high-performance liquid chromatography over Column: Boston Green ODS 150*30 mm*5 um to give the title compound N-(2-(5-azaspiro[3.4]octan-5-yl)ethyl)-6-methyl-5-((1-methyl-6-(pyrimidin-5-ylamino)-1H-pyrazolo[3,4-d]pyrimidin-3-yl)amino)nicotinamide (49.7 mg, 26%) as a yellow solid. LCMS (ESI): mass calcd. for C₂₅H₃₁N₁₁O, 501.587; m/z found, 502.4 [M+H]⁺. ¹H NMR (400 MHz, METHANOL-d₄) δ 9.20 (s, 2H), 8.93 (s, 1H), 8.85 (s, 1H), 8.68 (s, 1H), 8.40 (s, 1H), 3.80 (s, 3H), 3.77-3.73 (m, 1H), 3.62-3.38 (m, 3H), 3.32-3.25 (m, 2H), 3.13 (br s, 1H), 2.60 (s, 3H), 1.99-1.74 (m, 4H), 1.72-1.55 (m, 2H), 1.40 (br d, J=6.4 Hz, 3H).

### Example 6. N-(2-(2-azabicyclo[2.2.2]octan-2-yl)ethyl)-6-methyl-5-((1-methyl-6-(pyrimidin-5-ylamino)-1H-pyrazolo[3,4-d]pyrimidin-3-yl)amino)nicotinamide

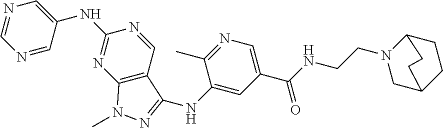 [SMILES: Cc1ncc(C(=O)NCCN2CC3CCC2CC3)cc1Nc1nn(C)c2nc(Nc3cncnc3)ncc12]

### Step a: 2-(2-azabicyclo[2.2.2]octan-2-yl)acetonitrile

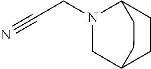 [SMILES: N#CCN1CC2CCC1CC2]

To a solution of 2-azabicyclo[2.2.2]octane (500 mg, 4.5 mmol) and potassium carbonate (1.4 g, 9.9 mmol) in N,N-dimethylformamide (10 mL) was added 2-bromoacetonitrile (593 mg, 4.9 mmol) at room-temperature. The resulting mixture was stirred at 60° C. for 12 h before cooling to room temperature. The resulting mixture was quenched with water (10 mL) and extracted with ethyl acetate (10 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=7:3) to give the title compound 2-(2-azabicyclo[2.2.2]octan-2-yl)acetonitrile (500 mg, 74%) as a colorless oil.

### Step b: 2-(2-azabicyclo[2.2.2]octan-2-yl)ethanamine

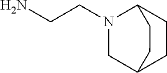 [SMILES: NCCN1CC2CCC1CC2]

To a solution of 2-(2-azabicyclo[2.2.2]octan-2-yl)acetonitrile (450 mg, 2.9 mmol) in THF (10 mL) was added lithium aluminium hydride (170 mg, 4.5 mmol) by portions at 0° C. (ice/water). The resulting mixture was stirred at 20° C. for 90 minutes before quenched with water (170 mg) at 0° C. The reaction mixture was filtered. And the filtrate was concentrated to dryness under reduced pressure to afford the crude product 2-(2-azabicyclo[2.2.2]octan-2-yl)ethanamine as a colorless oil. ¹H NMR (400 MHz, METHANOL-d₄) δ 2.57-2.73 (m, 4H), 2.47-2.55 (m, 2H), 1.79-1.94 (m, 2H), 1.34-1.63 (m, 8H).

### Step c: N-(2-(2-azabicyclo[2.2.2]octan-2-yl)ethyl)-6-methyl-5-((1-methyl-6-(pyrimidin-5-ylamino)-1H-pyrazolo[3,4-d]pyrimidin-3-yl)amino)nicotinamide

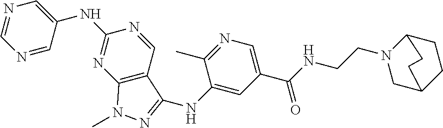 [SMILES: Cc1ncc(C(=O)NCCN2CC3CCC2CC3)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino)-nicotinic acid (130 mg, 0.31 mmol), HATU (232 mg, 0.61 mmol) and N,N-diisopropylethylamine (158 mg, 1.2 mmol) in N,N-dimethylformamide (4 mL) was added 2-(2-azabicyclo[2.2.2]octan-2-yl)ethanamine (47 mg, 0.31 mmol). The mixture stirred at 20° C. for 16 h and then concentrated under vacuum to give the crude product, which was purified by preparative high-performance liquid chromatography over Column: Waters Xbridge BEH C18 100*30 mm*10 um to give the title compound N-(2-(2-azabicyclo[2.2.2]octan-2-yl)ethyl)-6-methyl-5-((1-methyl-6-(pyrimidin-5-ylamino)-1H-pyrazolo[3,4-d]pyrimidin-3-yl)amino)nicotinamide (28 mg, 17%) as a brown solid. LCMS (ESI): mass calcd. for C₂₆H₃₁N₁₁O, 513.597; m/z found, 514.4 [M+H]⁺. ¹H NMR (400 MHz, METHANOL-d₄) δ 9.32 (s, 2H), 9.00 (s, 1H), 8.72-8.81 (m, 2H), 8.46 (d, J=1.76 Hz, 1H), 3.87 (s, 3H), 3.53 (br t, J=7.06 Hz, 2H), 2.98 (br s, 2H), 2.83-2.88 (m, 2H), 2.62-2.68 (m, 3H), 2.01 (br s, 2H), 1.53-1.70 (m, 8H).

### Example 7. N-(2-(7-azaspiro[3.5]nonan-7-yl)ethyl)-6-methyl-5-((1-methyl-6-(pyrimidin-5-ylamino)-1H-pyrazolo[3,4-d]pyrimidin-3-yl)amino)nicotinamide

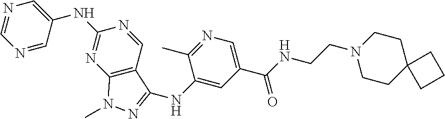 [SMILES: Cc1ncc(C(=O)NCCN2CCC3(CCC3)CC2)cc1Nc1nn(C)c2nc(Nc3cncnc3)ncc12]

### Step a: tert-butyl (2-(7-azaspiro[3.5]nonan-7-yl)ethyl)carbamate

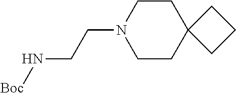 [SMILES: CC(C)(C)OC(=O)NCCN1CCC2(CCC2)CC1]

To a solution of 7-azaspiro[3.5]nonane hydrochloride (200 mg, 1.2 mmol) and potassium carbonate (341 mg, 2.4 mmol) in acetonitrile (3 mL) was added tert-butyl (2-bromoethyl)carbamate (277 mg, 1.2 mmol) at room-temperature. The resulting mixture was stirred at 80° C. for 12 h before cooling to room temperature. The reaction was concentrated under reduced pressure to give crude product, which was purified by column chromatography over silica gel (eluent: ethyl acetate:methanol=10:1) to give the title compound tert-butyl (2-(7-azaspiro[3.5]nonan-7-yl)ethyl)carbamate (290 mg, 100%) as a white solid. LCMS (ESI): mass calcd. for C₁₅H₂₈N₂O₂, 268.3; m/z found, 269.1 [M+H]⁺. ¹H NMR (400 MHz, CHLOROFORM-d) δ 3.15 (br s, 2H), 2.15-2.41 (m, 6H), 1.78 (br d, J=6.17 Hz, 2H), 1.66 (br d, J=4.85 Hz, 4H), 1.52 (br s, 4H), 1.39 (br s, 9H).

### Step b: 2-(7-azaspiro[3.5]nonan-7-yl)ethanamine

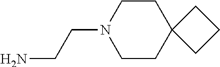 [SMILES: NCCN1CCC2(CCC2)CC1]

To a solution of tert-butyl (2-(7-azaspiro[3.5]nonan-7-yl)ethyl)carbamate (270 mg, 1.0 mmol) in THE (5 mL) was added HCl/MeOH (5 mL, 4 M) at 0° C. The resulting mixture was stirred at 20° C. for 2 hours. Then the reaction mixture was concentrated under reduced pressure to afford the crude product 2-(7-azaspiro[3.5]nonan-7-yl)ethanamine as a colorless oil. ¹H NMR (400 MHz, METHANOL-d₄) δ 3.50 (br d, J=12.13 Hz, 2H), 3.32-3.45 (m, 4H), 3.29 (dt, J=3.31, 1.65 Hz, 2H), 2.95-3.08 (m, 2H), 1.68-2.09 (m, 10H).

### Step c: N-(2-(7-azaspiro[3.5]nonan-7-yl)ethyl)-6-methyl-5-((1-methyl-6-(pyrimidin-5-ylamino)-1H-pyrazolo[3,4-d]pyrimidin-3-yl)amino)nicotinamide

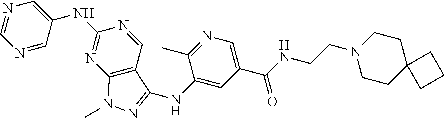 [SMILES: Cc1ncc(C(=O)NCCN2CCC3(CCC3)CC2)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino)nicotinic acid (68 mg, 0.18 mmol), 2-(7-azaspiro[3.5]nonan-7-yl)ethanamine (36 mg, 0.18 mmol) and N,N-diisopropylethylamine (0.12 mL, 0.72 mmol) in N,N-dimethylformamide (4 mL) was added HATU (136 mg, 0.36 mmol). The mixture stirred at 25° C. for 16 h and then concentrated under vacuum to give the crude product, which was purified by preparative high-performance liquid chromatography over Column:Phenomenex Gemini NX—C18(75*30 mm*3 um) to give the title compound N-(2-(7-azaspiro[3.5]nonan-7-yl)ethyl)-6-methyl-5-((1-methyl-6-(pyrimidin-5-ylamino)-1H-pyrazolo[3,4-d]pyrimidin-3-yl)amino)nicotinamide (45 mg, 42%) as a white solid. LCMS (ESI): mass calcd. for C₂₇H₃₃N₁₁O, 527.6; m/z found, 528.3 [M+H]⁺. ¹H NMR (400 MHz, METHANOL-d₄) δ 9.36 (s, 2H), 9.04 (s, 1H), 8.85 (d, J=2.03 Hz, 1H), 8.81 (s, 1H), 8.49-8.56 (m, 1H), 3.91 (s, 3H), 3.69 (t, J=6.32 Hz, 2H), 2.95 (br s, 2H), 2.69 (s, 3H), 2.34-2.54 (m, 2H), 1.92-2.00 (m, 2H), 1.87 (br d, J=7.15 Hz, 4H), 1.81 (br s, 4H), 1.28-1.67 (m, 2H).

### Example 8. N-(2-(3,3-dimethylpyrrolidin-1-yl)ethyl)-6-methyl-5-((1-methyl-6-(pyrimidin-5-ylamino)-1H-pyrazolo[3,4-d]pyrimidin-3-yl)amino)nicotinamide

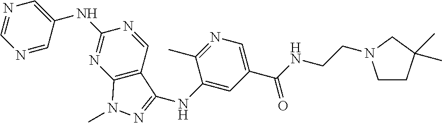 [SMILES: Cc1ncc(C(=O)NCCN2CCC(C)(C)C2)cc1Nc1nn(C)c2nc(Nc3cncnc3)ncc12]

### Step a: tert-butyl (2-(3,3-dimethylpyrrolidin-1-yl)ethyl)carbamate

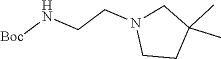 [SMILES: CC1(C)CCN(CCNC(=O)OC(C)(C)C)C1]

To a solution of 3,3-dimethylpyrrolidine hydrochloride (500 mg, 3.7 mmol) and potassium carbonate (1 g, 7.4 mmol) in acetonitrile (3 mL) was added tert-butyl (2-bromoethyl)carbamate (826 mg, 3.7 mmol) at room-temperature. The resulting mixture was stirred at 80° C. for 12 h before cooling to room temperature. The reaction was concentrated under reduced pressure to give crude product, which was purified by column chromatography over silica gel (eluent: ethyl acetate:methanol=10:1) to give the title compound tert-butyl (2-(3,3-dimethylpyrrolidin-1-yl)ethyl)carbamate (740 mg, 83%) as a yellow solid. LCMS (ESI): mass calcd. for C₁₃H₂₆N₂O₂, 242.3; m/z found, 243.2 [M+H]⁺. ¹H NMR (400 MHz, METHANOL-d₄) δ 3.14 (t, J=7.06 Hz, 2H), 2.65 (t, J=6.95 Hz, 2H), 2.51 (t, J=7.06 Hz, 2H), 2.38 (s, 2H), 1.59 (t, J=6.95 Hz, 2H), 1.41 (s, 9H), 1.07 (s, 6H).

### Step b: 2-(3,3-dimethylpyrrolidin-1-yl)ethanamine

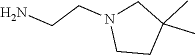

To a solution of (2-(7-azaspiro[3.5]nonan-7-yl)ethyl)carbamate (700 mg, 2.8 mmol) in HCl/dioxane (10 mL, 4M) at 0° C. The resulting mixture stirred at 20° C. for 2 hours. Then the reaction mixture concentrated under reduced pressure to afford the crude product 2-(3,3-dimethylpyrrolidin-1-yl)ethanamine as a yellow oil. ¹H NMR (400 MHz, METHANOL-d₄) δ 3.51-3.58 (m, 4H), 3.36-3.41 (m, 2H), 3.27-3.32 (m, 2H), 1.83-2.11 (m, 2H), 1.22 (d, J=4.41 Hz, 6H).

### Step c: N-(2-(3,3-dimethylpyrrolidin-1-yl)ethyl)-6-methyl-5-((1-methyl-6-(pyrimidin-5-ylamino)-1H-pyrazolo[3,4-d]pyrimidin-3-yl)amino)nicotinamide

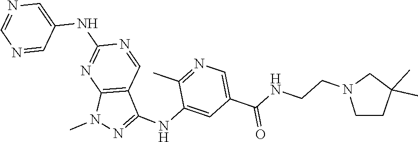 [SMILES: Cc1ncc(C(=O)NCCN2CCC(C)(C)C2)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino) nicotinic acid (130 mg, 0.3 mmol), 2-(3,3-dimethylpyrrolidin-1-yl)ethanamine (60 mg, 0.33 mmol) and N,N-diisopropylethylamine (0.2 mL, 1.2 mmol) in N,N-dimethylformamide (10 mL) was added HATU (233 mg, 0.61 mmol).

The mixture stirred at 25° C. for 16 h and then concentrated under vacuum to give the crude product, which was purified by preparative high-performance liquid chromatography over Column: Boston Prime C18 150*30 mm*5 um to give the title compound N-(2-(3,3-dimethylpyrrolidin-1-yl)ethyl)-6-methyl-5-((1-methyl-6-(pyrimidin-5-ylamino)-1H-pyrazolo[3,4-d]pyrimidin-3-yl)amino)nicotinamide (25 mg, 16%) as a yellow solid. LCMS (ESI): mass calcd. for C₂₅H₃₁N₁₁O, 501.5; m/z found, 502.2 [M+H]⁺. ¹H NMR (400 MHz, METHANOL-d₄) δ 9.33 (s, 2H), 9.02 (s, 1H), 8.83 (d, J=1.76 Hz, 1H), 8.79 (s, 1H), 8.48 (d, J=1.76 Hz, 1H), 3.89 (s, 3H), 3.59 (t, J=6.78 Hz, 2H), 2.77-3.00 (m, 4H), 2.55-2.70 (m, 5H), 1.71 (t, J=7.03 Hz, 2H), 1.14 (s, 6H).

### Example 9. N-(2-(cyclopentyl(methyl)amino)ethyl)-6-methyl-5-((1-methyl-6-(pyrimidin-5-ylamino)-1H-pyrazolo[3,4-d]pyrimidin-3-yl)amino)nicotinamide

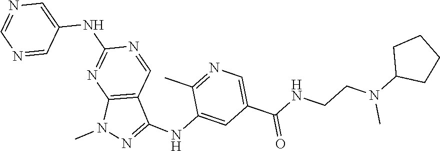 [SMILES: Cc1ncc(C(=O)NCCN(C)C2CCCC2)cc1Nc1nn(C)c2nc(Nc3cncnc3)ncc12]

### Step a: tert-butyl (2-(cyclopentyl(methyl)amino)ethyl)carbamate

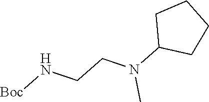 [SMILES: CN(CCNC(=O)OC(C)(C)C)C1CCCC1]

To a solution of N-methylcyclopentanamine hydrochloride (800 mg, 5.90 mmol) in acetonitrile (10 mL) was added tert-butyl (2-bromoethyl)carbamate (1.3 g, 5.9 mmol) and K₂CO₃ (1.63 g, 11.8 mmol) at room temperature. The reaction mixture was stirred at 80° C. overnight. The mixture was cooled to room temperature and filtered. The filtrate was evaporated to afford crude product. The crude product was purified by column chromatography over silica gel (eluent: dichloromethane/MeOH=100/0-97/3). The desired fractions were evaporated in vacuum to give the title compound tert-butyl (2-(cyclopentyl(methyl)amino)ethyl)carbamate (1.1 g, 76%) as colorless oil. ¹H NMR (400 MHz, DMSO-d₆) δ 4.93 (br s, 1H), 3.13 (br d, J=5.48 Hz, 2H), 2.64 (br t, J=7.99 Hz, 1H), 2.43 (br t, J=5.90 Hz, 2H), 2.13 (s, 3H), 1.69-1.77 (m, 2H), 1.53-1.64 (m, 2H), 1.41-1.50 (m, 2H), 1.37 (s, 9H), 1.26-1.34 (m, 2H).

### Step b: N¹-cyclopentyl-N¹-methylethane-1,2-diamine

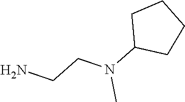

A solution of tert-butyl (2-(cyclopentyl(methyl)amino)ethyl)carbamate (200 mg, 0.83 mmol) in HCl/methanol (4 mL, 4M, 16 mmol) was stirred at room temperature for 1 hour. The mixture was evaporated to afford crude compound N¹-cyclopentyl-N¹-methylethane-1,2-diamine as colorless oil. The crude compound was used for nest step without purification. ¹H NMR (400 MHz, CHLOROFORM-d) δ 8.40-8.65 (m, 2H), 3.53-3.77 (m, 3H), 2.90-3.02 (m, 2H), 1.46-2.24 (m, 11H).

### Step c: N-(2-(cyclopentyl(methyl)amino)ethyl)-6-methyl-5-((1-methyl-6-(pyrimidin-5-ylamino)-1H-pyrazolo[3,4-d]pyrimidin-3-yl)amino)nicotinamide

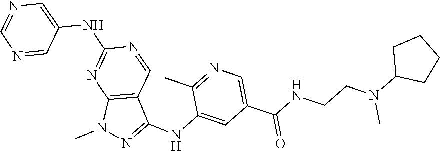 [SMILES: Cc1ncc(C(=O)NCCN(C)C2CCCC2)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino)nicotinic acid (140 mg, 0.32 mmol), DIEA (0.21 mL, 1.28 mmol) in DMF (2.8 mL) was added N¹-cyclopentyl-N¹-methylethane-1,2-diamine (75 mg, 0.42 mmol). The mixture was stirred at room temperature for 10 min. Then HATU (183 mg, 0.48 mmol) was added. The mixture was stirred at room temperature overnight. The reaction mixture was concentrated in vacuo to give the crude product. The crude product was purified by preparative high-performance liquid chromatography over Column: Boston Prime C18 150*30 mm*5 um to give the title compound N-(2-(cyclopentyl(methyl)amino)ethyl)-6-methyl-5-((1-methyl-6-(pyrimidin-5-ylamino)-1H-pyrazolo[3,4-d]pyrimidin-3-yl)amino)nicotinamide (55.1 mg, 34%) as brown solid. LCMS (ESI): mass calcd. for C₂₅H₃₁N₁₁O, 501.6; m/z found, 502.2 [M+H]⁺. ¹H NMR (400 MHz, METHANOL-d₄) δ 9.36 (s, 2H), 9.04 (s, 1H), 8.84 (d, J=1.91 Hz, 1H), 8.80 (s, 1H), 8.49 (d, J=1.91 Hz, 1H), 3.90 (s, 3H), 3.62 (t, J=6.79 Hz, 2H), 3.03 (br s, 1H), 2.87 (br s, 2H), 2.68 (s, 3H), 2.51 (s, 3H), 1.97 (br s, 2H), 1.71-1.80 (m, 2H), 1.63 (br dd, J=7.63, 4.53 Hz, 2H) 1.48-1.57 (m, 2H).

### Example 10. 6-methyl-N-(2-(methyl(tetrahydro-2H-pyran-4-yl)amino)ethyl)-5-((1-methyl-6-(pyrimidin-5-ylamino)-1H-pyrazolo[3,4-d]pyrimidin-3-yl)amino)nicotinamide

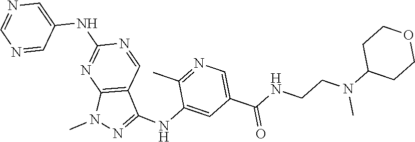 [SMILES: Cc1ncc(C(=O)NCCN(C)C2CCOCC2)cc1Nc1nn(C)c2nc(Nc3cncnc3)ncc12]

### Step a: tert-butyl (2-(methyl(tetrahydro-2H-pyran-4-yl)amino)ethyl)carbamate

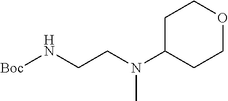 [SMILES: CN(CCNC(=O)OC(C)(C)C)C1CCOCC1]

To a solution of N-methyltetrahydro-2H-pyran-4-amine (1 g, 8.7 mmol) in acetonitrile (10 mL) was added tert-butyl (2-bromoethyl)carbamate (2.0 g, 8.7 mmol) and K₂CO₃ (2.4 g, 17.4 mmol) at room temperature. The reaction mixture was stirred at 80° C. overnight. The mixture was cooled to room temperature and filtered. The filtrate was evaporated to afford crude product. The crude product was purified by column chromatography over silica gel (eluent: dichloromethane/MeOH=100/0-97/3). The desired fractions were evaporated in vacuum to give the title compound tert-butyl (2-(methyl(tetrahydro-2H-pyran-4-yl)amino)ethyl)carbamate (1.9 g, 86%) as colorless oil. ¹H NMR (400 MHz, CHLOROFORM-d) δ 4.90 (br s, 1H), 3.95 (br dd, J=11.27, 4.35 Hz, 2H), 3.29 (td, J=11.65, 2.21 Hz, 2H), 3.11 (br d, J=5.25 Hz, 2H), 2.42-2.54 (m, 3H), 2.17 (s, 3H), 1.53-1.63 (m, 4H), 1.38 (s, 9H).

### Step b: N¹-methyl-N¹-(tetrahydro-2H-pyran-4-yl)ethane-1,2-diamine

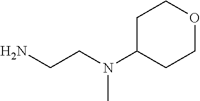

A solution of tert-butyl (2-(methyl(tetrahydro-2H-pyran-4-yl)amino)ethyl)carbamate (200 mg, 0.77 mmol) in HCl/methanol (4 mL, 4M, 16 mmol) was stirred at room temperature for 1 hour. The mixture was evaporated to afford crude compound N¹-methyl-N¹-(tetrahydro-2H-pyran-4-yl)ethane-1,2-diamine as colorless oil. The crude compound was used for nest step without purification.

### Step c: 6-methyl-N-(2-(methyl(tetrahydro-2H-pyran-4-yl)amino)ethyl)-5-((1-methyl-6-(pyrimidin-5-ylamino)-1H-pyrazolo[3,4-d]pyrimidin-3-yl)amino)nicotinamide

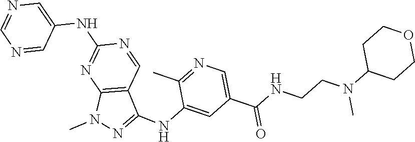 [SMILES: Cc1ncc(C(=O)NCCN(C)C2CCOCC2)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino)nicotinic acid (140 mg, 0.32 mmol), DIEA (0.21 mL, 1.3 mmol) in DMF (2.8 mL) was added N¹-methyl-N¹-(tetrahydro-2H-pyran-4-yl)ethane-1,2-diamine (81 mg, 0.42 mmol). The mixture was stirred at room temperature for 10 min. Then HATU (183 mg, 0.48 mmol) was added. The mixture was stirred at room temperature overnight. The reaction mixture was concentrated in vacuo to give the crude product. The crude product was purified by preparative high-performance liquid chromatography over Column: Boston Prime C18 150*30 mm*5 um to give the title compound: 6-methyl-N-(2-(methyl(tetrahydro-2H-pyran-4-yl)amino)ethyl)-5-((1-methyl-6-(pyrimidin-5-ylamino)-1H-pyrazolo[3,4-d]pyrimidin-3-yl)amino)nicotinamide (51.6 mg, 31%) as brown solid. LCMS (ESI): mass calcd. for C₂₅H₃₁N₁₁O₂, 517.6; m/z found, 518.2 [M+H]⁺. ¹H NMR (400 MHz, METHANOL-d₄) δ 9.35 (s, 2H), 9.03 (s, 1H), 8.82 (d, J=1.91 Hz, 1H), 8.80 (s, 1H), 8.48 (d, J=1.91 Hz, 1H), 4.00 (br dd, J=11.56, 4.17 Hz, 2H), 3.90 (s, 3H), 3.56 (t, J=6.79 Hz, 2H), 3.39-3.46 (m, 2H), 2.73-2.83 (m, 3H), 2.68 (s, 3H), 2.43 (s, 3H), 1.78-1.86 (m, 2H), 1.59 (br dd, J=12.34, 4.23 Hz, 2H).

### Example 11. (R)-6-methyl-5-((1-methyl-6-(pyrimidin-5-ylamino)-1H-pyrazolo[3,4-d]pyrimidin-3-yl)amino)-N-(2-(2-methylpiperidin-1-yl)ethyl)nicotinamide

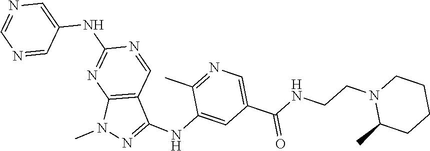 [SMILES: Cc1ncc(C(=O)NCCN2CCCC[C@H]2C)cc1Nc1nn(C)c2nc(Nc3cncnc3)ncc12]

### Step a: (R)-tert-butyl (2-(2-methylpiperidin-1-yl)ethyl)carbamate

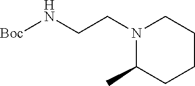 [SMILES: C[C@@H]1CCCCN1CCNC(=O)OC(C)(C)C]

To a solution of tert-butyl (2-bromoethyl)carbamate (2.5 g, 11.09 mmol) and potassium carbonate (519 mg, 9.9 mmol) in acetonitrile (5 mL) was added 2-methylpiperidine (1 g, 10.08 mmol) at room temperature. The resulting mixture was stirred at 50° C. for 16 h before cooling to room-temperature. The mixture was quenched with water (5 mL) and extracted with ethyl acetate (10 mL×3). The combined organic phases were dried over anhydrous Na₂SO₄ and filtered. The filtrate was concentrated under reduced pressure to give crude product, which was purified by column chromatography over silica gel (eluent: petroleum ether:ethyl acetate=3:1) to give the title compound (R)-tert-butyl (2-(2-methylpiperidin-1-yl)ethyl)carbamate (1.3 g, 48%) as a yellow oil. LCMS (ESI): mass calcd. for C₁₃H₂₆N₂O₂, 242.3; m/z found, 243.2 [M+H]⁺.

### Step b: (R)-2-(2-methylpiperidin-1-yl)ethanamine

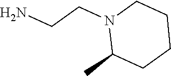

To a solution of (R)-tert-butyl (2-(2-methylpiperidin-1-yl)ethyl)carbamate (1.3 g, 5.3 mmol) in DCM (3 mL) was added HCl/MeOH (3 mL) at room temperature for 16 h. The reaction mixture was concentrated in vacuum to give the crude product (R)-2-(2-methylpiperidin-1-yl)ethanamine (1 g, 61%) as a yellow oil. LCMS (ESI): mass calcd. for C₈H₁₈N₂, 142.2; m/z found, 143.1 [M+H]⁺.

### Step c: (R)-6-methyl-5-((1-methyl-6-(pyrimidin-5-ylamino)-1H-pyrazolo[3,4-d]pyrimidin-3-yl)amino)-N-(2-(2-methylpiperidin-1-yl)ethyl)nicotinamide

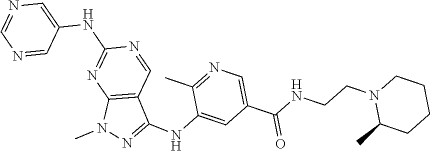 [SMILES: Cc1ncc(C(=O)NCCN2CCCC[C@H]2C)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino) nicotinic acid (100 mg, 0.27 mmol),2-(3H-[1,2,3]triazolo[4,5-b]pyridin-3-yl)-1,1,3,3-tetramethylisouronium hexafluorophosphate(V) (201 mg, 0.53 mmol), and N-ethyl-N-isopropylpropan-2-amine (136 mg, 1.06 mmol) in N,N-dimethylformamide (2 mL) was added (R)-2-(2-methylpiperidin-1-yl)ethanamine (57.02 mg, 0.26 mmol). The mixture was stirred at 50° C. for 2 hours. The reaction mixture was concentrated under vacuum to give the crude product, which was purified by preparative high-performance liquid chromatography over Column: Boston Prime C18 150*30 mm*5 um to give the title compound (R)-6-methyl-5-((1-methyl-6-(pyrimidin-5-ylamino)-1H-pyrazolo[3,4-d]pyrimidin-3-yl)amino)-N-(2-(2-methylpiperidin-1-yl)ethyl)nicotinamide (48 mg, 98%) as a white solid. LCMS (ESI): mass calcd. for C₂₅H₃₁N₁₁O, 501.6; m/z found, 502.2 [M+H]⁺. ¹H NMR (400 MHz, METHANOL-d₄) δ 9.34 (s, 2H), 9.04 (s, 1H), 8.80 (s, 1H), 8.47-8.52 (m, 2H), 3.90 (s, 3H), 3.77 (t, J=6.50 Hz, 2H), 3.56 (br d, J=12.40 Hz, 1H), 3.43-3.52 (m, 1H), 3.20-3.30 (m, 1H), 2.98-3.16 (m, 2H), 2.69 (s, 3H), 1.79-2.02 (m, 4H), 1.59-1.74 (m, 2H), 1.41 (d, J=6.44 Hz, 2H), 1.37-1.47 (m, 1H).

### Example 12. (S)-6-methyl-5-((1-methyl-6-(pyrimidin-5-ylamino)-1H-pyrazolo[3,4-d]pyrimidin-3-yl)amino)-N-(2-(3-methylpyrrolidin-1-yl)ethyl)nicotinamide

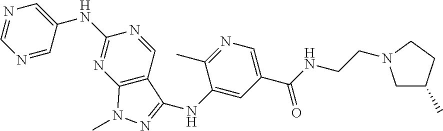 [SMILES: Cc1ncc(C(=O)NCCN2CC[C@H](C)C2)cc1Nc1nn(C)c2nc(Nc3cncnc3)ncc12]

### Step a: (S)-2-(3-methylpyrrolidin-1-yl)acetonitrile

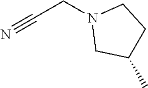

To a solution of (S)-3-methylpyrrolidine hydrochloride (900 mg, 5.69 mmol) and potassium carbonate (6.30 g, 45.55 mmol) in N,N-dimethylformamide (10 mL) was added 2-bromoacetonitrile (1.54 g, 22.77 mmol) at room temperature. The resulting mixture was stirred at 50° C. for 16 h before cooling to room-temperature. The resulting mixture was quenched with water (5 mL) and extracted with ethyl acetate (10 mL×3). The combined organic phases were dried over anhydrous Na₂SO₄ and filtered. The filtrate was concentrated under reduced pressure to give crude product, which was purified by column chromatography over silica gel (eluent: petroleum ether:ethyl acetate=3:1) to give the title compound (S)-2-(3-methylpyrrolidin-1-yl)acetonitrile (500 mg, 85%) as a yellow oil. ¹H NMR (400 MHz, METHANOL-d₄) δ 3.67-3.91 (m, 2H), 2.66-2.84 (m, 2H), 2.22-2.48 (m, 2H), 2.11 (br d, J=4.89 Hz, 1H), 1.45 (br d, J=3.81 Hz, 1H), 1.01-1.30 (m, 4H).

### Step b: (S)-2-(3-methylpyrrolidin-1-yl)ethanamine

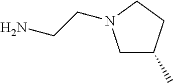

To a solution of (S)-2-(3-methylpyrrolidin-1-yl)acetonitrile(400 mg, 3.22 mmol) in THF (5 mL) was added lithium aluminium hydride (147 mg, 3.87 mmol) by portions at 0° C. (ice/water), and the resulting mixture was stirred for 16 h at 25° C. After cooled to 0° C., the reaction mixture was quenched with water (250 mg) and filtered. The filtrate was then concentrated to dryness under reduced pressure to afford the crude product (S)-2-(3-methylpyrrolidin-1-yl)ethanamine (200 mg, 48%) as a colorless oil. ¹H NMR (400 MHz, METHANOL-d₄) δ 3.32 (br s, 1H), 2.85-3.04 (m, 1H), 2.73-2.84 (m, 2H), 2.56 (br d, J=7.03 Hz, 2H), 2.28 (br d, J=6.32 Hz, 2H), 1.88-2.20 (m, 2H), 1.40 (br d, J=6.68 Hz, 1H), 1.06 (br d, J=6.56 Hz, 3H).

### Step c: (S)-6-methyl-5-((1-methyl-6-(pyrimidin-5-ylamino)-1H-pyrazolo[3,4-d]pyrimidin-3-yl)amino)-N-(2-(3-methylpyrrolidin-1-yl)ethyl)nicotinamide

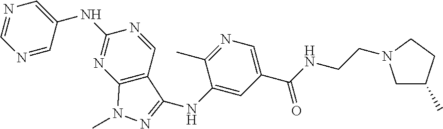 [SMILES: Cc1ncc(C(=O)NCCN2CC[C@H](C)C2)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino) nicotinic acid (150 mg, 0.35 mmol), 2-(3H-[1,2,3]triazolo[4,5-b]pyridin-3-yl)-1,1,3,3-tetramethylisouronium hexafluorophosphate(V) (265 mg, 0.7 mmol), and N-ethyl-N-isopropylpropan-2-amine (180 mg, 1.4 mmol) in N,N-dimethylformamide (5 mL) was added (S)-2-(3-methylpyrrolidin-1-yl)ethanamine (45 mg, 0.35 mmol). The mixture was stirred at 50° C. for 2 hours. The reaction mixture was concentrated under vacuum to give the crude product, which was purified by preparative high-performance liquid chromatography over Column: Boston Prime C18 150*30 mm*5 um to give the title compound (S)-6-methyl-5-((1-methyl-6-(pyrimidin-5-ylamino)-1H-pyrazolo[3,4-d]pyrimidin-3-yl)amino)-N-(2-(3-methylpyrrolidin-1-yl)ethyl)nicotinamide (33 mg, 19%) as a white solid. LCMS (ESI): mass calcd. for C₂₄H₂₉N₁₁O, 487.56; m/z found, 488.2 [M+H]⁺. ¹H NMR (400 MHz, METHANOL-d₄) δ 9.22 (s, 2H), 8.95 (s, 1H), 8.86 (s, 1H), 8.70 (s, 1H), 8.43 (s, 1H), 3.83 (s, 3H), 3.78 (br s, 2H), 3.63-3.72 (m, 1H), 3.52 (br d, J=6.53 Hz, 2H), 3.45 (br s, 2H), 2.98 (br t, J=9.91 Hz, 1H), 2.63 (s, 3H), 2.46-2.58 (m, 1H), 2.22-2.35 (m, 1H), 1.66-1.79 (m, 1H), 1.18 (d, J=6.78 Hz, 3H).

### Example 13. N-(2-(7-oxa-4-azaspiro[2.5]octan-4-yl)ethyl)-6-methyl-5-((1-methyl-6-(pyrimidin-5-ylamino)-1H-pyrazolo[3,4-d]pyrimidin-3-yl)amino)nicotinamide

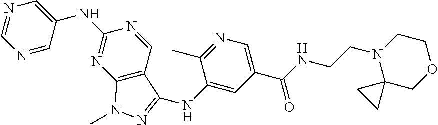 [SMILES: Cc1ncc(C(=O)NCCN2CCOCC23CC3)cc1Nc1nn(C)c2nc(Nc3cncnc3)ncc12]

### Step a: 2-(7-oxa-4-azaspiro[2.5]octan-4-yl)acetonitrile

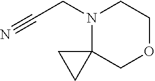 [SMILES: N#CCN1CCOCC12CC2]

To a solution of 7-oxa-4-azaspiro[2.5]octane hydrochloride (150 mg, 1.0 mmol) and potassium carbonate (346 mg, 2.5 mmol) in acetonitrile (6 mL) was added 2-bromoacetonitrile (69 ul, 1.1 mmol) at room-temperature. To a solution of 1,4-oxazepane hydrochloride (800 mg, 7.9 mmol) and potassium carbonate (2.73 g, 19.7 mmol) in acetonitrile (10 mL) was added 2-bromoacetonitrile (1 g, 8.7 mmol) at room temperature. The resulting mixture was stirred at 50° C. for 12 h before cooling to room-temperature. The reaction mixture was filtered. The residue was washed with ethyl acetate (10 mL×3) and concentrated under reduced pressure to give crude product, which was purified by column chromatography over silica gel (eluent: petroleum ether:ethyl acetate=3:1) to give the title compound 2-(7-oxa-4-azaspiro[2.5]octan-4-yl)acetonitrile (150 mg, 98%) as a colorless oil. LCMS (ESI): mass calcd. for C₈H₁₂N₂O, 152.19; m/z found, 153.05 [M+H]⁺. ¹H NMR (400 MHz, CDCl₃) δ 3.80-3.74 (m, 2H), 3.63 (s, 2H), 3.47 (s, 2H), 3.12-3.04 (m, 2H), 0.86-0.78 (m, 2H), 0.61-0.54 (m, 2H).

### Step b:_2-(4-azaspiro[2.4]heptan-4-yl)ethan-1-amine

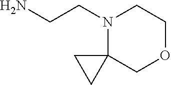 [SMILES: NCCN1CCOCC12CC2]

To a solution of 2-(7-oxa-4-azaspiro[2.5]octan-4-yl)acetonitrile (130 mg, 0.85 mmol) in THF (10 mL) was added lithiumaluminium hydride (65 mg, 1.71 mmol) by portions at 0° C. (ice/water), and the resulting mixture was stirred for 90 minutes at 25° C. After cooled to 0° C., the reaction mixture was quenched with water (65 mg) and filtered. The filtrate was then concentrated to dryness under reduced pressure to afford the crude product 2-(4-azaspiro[2.4]heptan-4-yl)ethan-1-amine (130 mg, 97%) as a colorless oil. LCMS (ESI): mass calcd. for C₈H₁₆N₂O, 156.2; m/z found, 156.9[M+H]⁺. ¹H NMR (400 MHz, METHANOL-d₄) δ 3.74-3.69 (m, 2H), 3.46 (s, 2H), 2.95-2.91 (m, 2H), 2.81 (t, J=6.3 Hz, 2H), 2.66-2.57 (m, 2H), 0.70 (t, J=5.5 Hz, 2H), 0.53-0.48 (m, 2H).

### Step c: N-(2-(7-oxa-4-azaspiro[2.5]octan-4-yl)ethyl)-6-methyl-5-((1-methyl-6-(pyrimidin-5-ylamino)-1H-pyrazolo[3,4-d]pyrimidin-3-yl)amino)nicotinamide

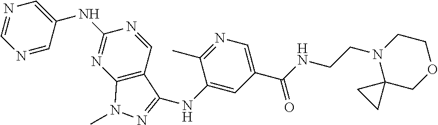 [SMILES: Cc1ncc(C(=O)NCCN2CCOCC23CC3)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino) nicotinic acid (130 mg, 0.34 mmol), HATU (236 mg, 0.62 mmol) and N,N-diisopropylethylamine (178 mg, 1.4 mmol) in N,N-dimethylformamide (6 mL) was added 2-(7-oxa-4-azaspiro[2.5]octan-4-yl)ethanamine (65 mg, 0.41 mmol). The mixture stirred at 25° C. for 2 h and then concentrated under vacuum to give the crude product, which was purified by preparative high-performance liquid chromatography over Column: Welch Xtimate C18 150*25 mm*5 um to give the title compound N-(2-(7-oxa-4-azaspiro[2.5]octan-4-yl)ethyl)-6-methyl-5-((1-methyl-6-(pyrimidin-5-ylamino)-1H-pyrazolo[3,4-d]pyrimidin-3-yl)amino)nicotinamide (58 mg, 31%) as a pale brown solid. LCMS (ESI): mass calcd. for C₂₅H₂₉N₁₁O₂, 515.2; m/z found, 516.2 [M+H]⁺. ¹H NMR (400 MHz, METHANOL-d₄) δ 9.50 (s, 1H), 9.39 (br s, 2H), 9.13 (s, 1H), 8.85 (s, 1H), 8.56 (s, 1H), 4.12 (br s, 2H), 3.98 (s, 3H), 3.88-3.56 (m, 8H), 2.89 (s, 3H), 1.34 (br s, 2H), 1.12 (br s, 2H).

### Example 14. N-(2-((2S,6R)-2,6-dimethylpiperidin-1-yl)ethyl)-6-methyl-5-((1-methyl-6-(pyrimidin-5-ylamino)-1H-pyrazolo[3,4-d]pyrimidin-3-yl)amino)nicotinamide

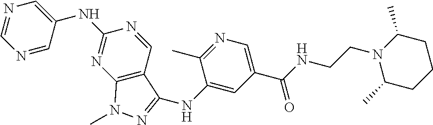 [SMILES: Cc1ncc(C(=O)NCCN2[C@H](C)CCC[C@@H]2C)cc1Nc1nn(C)c2nc(Nc3cncnc3)ncc12]

### Step a: 2-((2S,6R)-2,6-dimethylpiperidin-1-yl)acetonitrile

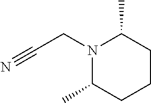

To a solution of (2S,6R)-2,6-dimethylpiperidine (1 g, 8.83 mmol) and potassium carbonate (2.44 g, 17.67 mmol) in DMF (10 mL) was added 2-bromoacetonitrile (0.9 mL, 13.25 mmol) at room-temperature. The resulting mixture was stirred at 50° C. for 12 h before cooling to room-temperature. The reaction mixture was filtered. The residue was washed with ethyl acetate (10 mL×3) and concentrated under reduced pressure to give crude product, which was purified by column chromatography over silica gel (eluent: petroleum ether:ethyl acetate=3:1) to give the title compound 2-((2S,6R)-2,6-dimethylpiperidin-1-yl)acetonitrile (800 mg, 60%) as a colorless oil. ¹H NMR (400 MHz, CDCl₃) δ 3.75 (s, 2H), 2.34-2.48 (m, 2H), 1.64 (br d, J=9.92 Hz, 3H), 1.21-1.48 (m, 3H), 1.09 (d, J=6.17 Hz, 6H).

### Step b: 2-((2S,6R)-2,6-dimethylpiperidin-1-yl)ethan-1-amine

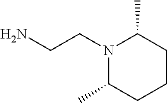

To a solution of 2-((2S,6R)-2,6-dimethylpiperidin-1-yl)acetonitrile (800 mg, 5.25 mmol) in THF (10 mL) was added lithium aluminium hydride (240 mg, 6.31 mmol) by portions at 0° C. (ice/water), and the resulting mixture was stirred for 90 minutes at 25° C. After cooled to 0° C., the reaction mixture was quenched with water (250 mg) and filtered. The filtrate was then concentrated to dryness under reduced pressure to afford the crude product 2-((2S,6R)-2,6-dimethylpiperidin-1-yl)ethan-1-amine (800 mg, 97%) as a colorless oil. ¹H NMR (400 MHz, CDCl₃) δ 2.65-2.73 (m, 2H), 2.57-2.65 (m, 2H), 2.32-2.45 (m, 2H), 1.41-1.65 (m, 3H), 1.14-1.24 (m, 3H), 1.06 (d, J=6.39 Hz, 6H).

### Step c: N-(2-((2S,6R)-2,6-dimethylpiperidin-1-yl)ethyl)-6-methyl-5-((1-methyl-6-(pyrimidin-5-ylamino)-1H-pyrazolo[3,4-d]pyrimidin-3-yl)amino)nicotinamide

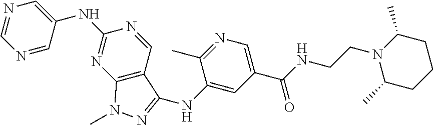 [SMILES: Cc1ncc(C(=O)NCCN2[C@H](C)CCC[C@@H]2C)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino) nicotinic acid (135 mg, 0.31 mmol), 2-((2S,6R)-2,6-dimethylpiperidin-1-yl)ethanamine (53 mg, 0.34 mmol) and N,N-diisopropylethylamine (0.2 mL, 1.2 mmol) in N,N-dimethylformamide (10 mL) was added HATU (235 mg 0.62 mmol). The mixture stirred at 25° C. for 16 h and then concentrated under vacuum to give the crude product, which was purified by preparative high-performance liquid chromatography over Column: Phenomenex Gemini NX—C18 (75*30 mm*3 um) to give the title compound N-(2-((2S,6R)-2,6-dimethylpiperidin-1-yl)ethyl)-6-methyl-5-((1-methyl-6-(pyrimidin-5-ylamino)-1H-pyrazolo[3,4-d]pyrimidin-3-yl)amino)nicotinamide (38 mg, 23%) as a yellow solid. LCMS (ESI): mass calcd. for C₂₆H₃₃N₁₁O, 515.6; m/z found, 516.3 [M+H]⁺. ¹H NMR (400 MHz, METHANOL-d₄) δ 9.32 (s, 2H), 8.99 (s, 1H), 8.67-8.82 (m, 2H), 8.44 (d, J=1.76 Hz, 1H), 4.56 (br s, 2H), 3.87 (s, 3H), 3.48 (br d, J=8.16 Hz, 2H), 2.98 (s, 2H), 2.64 (s, 3H), 1.63 (br d, J=15.88 Hz, 3H), 1.29-1.49 (m, 3H), 1.23 (br d, J=6.17 Hz, 6H).

### Example 15. N-(2-(diisopropylamino)ethyl)-6-methyl-5-((1-methyl-6-(pyrimidin-5-ylamino)-1H-pyrazolo[3,4-d]pyrimidin-3-yl)amino)nicotinamide

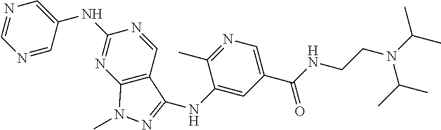 [SMILES: Cc1ncc(C(=O)NCCN(C(C)C)C(C)C)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino) nicotinic acid (130 mg, 0.3 mmol), N¹,N¹-diisopropylethane-1,2-diamine (47 mg, 0.33 mmol) and N,N-diisopropylethylamine (0.2 mL, 1.2 mmol) in N,N-dimethylformamide (10 mL) was added HATU (227 mg, 0.6 mmol). The mixture stirred at 25° C. for 16 h and then concentrated under vacuum to give the crude product, which was purified by preparative high-performance liquid chromatography over Column: Prime C18 150*30 mm*5 um to give the title compound N-(2-(diisopropylamino)ethyl)-6-methyl-5-((1-methyl-6-(pyrimidin-5-ylamino)-1H-pyrazolo[3,4-d]pyrimidin-3-yl)amino)nicotinamide (33 mg, 21%) as a yellow solid. LCMS (ESI): mass calcd. for C₂₅H₃₃N₁₀, 503.6; m/z found, 504.3 [M+H]⁺. ¹H NMR (400 MHz, METHANOL-d₄) δ 9.32 (s, 2H), 8.99 (s, 1H), 8.76 (s, 2H), 8.44 (d, J=1.98 Hz, 1H), 4.56 (br s, 2H), 3.87 (s, 3H), 3.41 (br s, 2H), 3.11 (br s, 1H), 2.73 (br s, 1H), 2.64 (s, 3H), 0.90-1.22 (m, 12H).

### Example 16. 1-(2-(2,2-dimethylpyrrolidin-1-yl)ethyl)-3-(6-methyl-5-((1-methyl-6-(pyrimidin-5-ylamino)-1H-pyrazolo[3,4-d]pyrimidin-3-yl)amino)pyridin-3-yl)urea

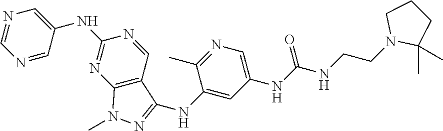 [SMILES: Cc1ncc(NC(=O)NCCN2CCCC2(C)C)cc1Nc1nn(C)c2nc(Nc3cncnc3)ncc12]

### Step a: 4-nitrophenyl (2-(2,2-dimethylpyrrolidin-1-yl)ethyl)carbamate

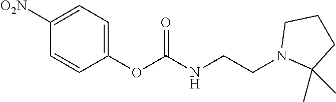 [SMILES: CC1(C)CCCN1CCNC(=O)Oc1ccc([N+](=O)[O-])cc1]

To a solution of 2-(2,2-dimethylpyrrolidin-1-yl)ethanamine (300 mg, 2.1 mmol) in dichloromethane (10 mL) was added 4-nitrophenylcarbamic chloride (425 mg, 2.1 mmol) at −20° C. The resulting mixture was stirred at 0° C. for 1.5 h. The reaction mixture was concentrated under reduced pressure give the title compound 4-nitrophenyl (2-(2,2-dimethylpyrrolidin-1-yl)ethyl)carbamate (300 mg, 36%), which was no purified and was used to next step as a yellow oil. LCMS (ESI): mass calcd. for C₁₅H₂₁N₃O₄, 307.3; m/z found, 307.9 [M+H]⁺.

### Step b: 1-(2-(2,2-dimethylpyrrolidin-1-yl)ethyl)-3-(6-methyl-5-((1-methyl-6-(pyrimidin-5-ylamino)-1H-pyrazolo[3,4-d]pyrimidin-3-yl)amino)pyridin-3-yl)urea

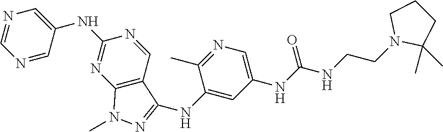 [SMILES: Cc1ncc(NC(=O)NCCN2CCCC2(C)C)cc1Nc1nn(C)c2nc(Nc3cncnc3)ncc12]

To a solution of 4-nitrophenyl (2-(2,2-dimethylpyrrolidin-1-yl)ethyl)carbamate (51 mg, 0.13 mmol) and N,N-dimethylpyridin-4-amine (39 mg, 0.32 mmol) in acetonitrile (2 mL) was added N³-(5-amino-2-methylpyridin-3-yl)-1-methyl-N⁶-(pyrimidin-5-yl)-1H-pyrazolo[3,4-d]pyrimidine-3,6-diamine (70 mg, 0.13 mmol) at 25° C.

The resulting mixture was stirred at 80° C. for 12 h. The reaction mixture was concentrated under reduced pressure give crude product as black oil, which was purified by preparative high-performance liquid chromatography over column: Phenomenex Gemini-NX 150*30 mm*5 um to give the title compound 1-(2-(2,2-dimethylpyrrolidin-1-yl)ethyl)-3-(6-methyl-5-((1-methyl-6-(pyrimidin-5-ylamino)-1H-pyrazolo[3,4-d] pyrimidin-3-yl)amino)pyridin-3-yl)urea (3.8 mg, 5%) as a yellow solid. LCMS (ESI): mass calcd. for C₂₅H₃₂N₁₂O, 516.6; m/z found, 517.5 [M+H]⁺. ¹H NMR (400 MHz, METHANOL-d₆) 6 9.36 (s, 2H), 8.99 (s, 1H), 8.81 (s, 1H), 8.47 (d, J=2.3 Hz, 1H), 8.19 (d, J=2.3 Hz, 1H), 3.89 (s, 3H), 3.37 (br s, 2H), 2.90 (br s, 2H), 2.64 (br s, 2H), 2.54 (s, 3H), 1.89-1.79 (m, 2H), 1.73 (br d, J=8.8 Hz, 2H), 1.07 (s, 6H).

### Example 17. N-(2-(1,4-oxazepan-4-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

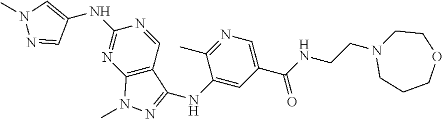 [SMILES: Cc1ncc(C(=O)NCCN2CCCOCC2)cc1Nc1nn(C)c2nc(Nc3cnn(C)c3)ncc12]

### Step a: 2-(1,4-oxazepan-4-yl)acetonitrile

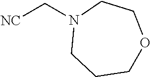

To a solution of 1,4-oxazepane hydrochloride (800 mg, 7.9 mmol) and potassium carbonate (2.73 g, 19.7 mmol) in acetonitrile (10 mL) was added 2-bromoacetonitrile (1 g, 8.7 mmol) at room temperature. The resulting mixture was stirred at 50° C. for 12 h before cooling to room-temperature. The mixture was filtered. The residue was washed with ethyl acetate (10 mL×3) and concentrated under reduced pressure to give crude product, which was purified by column chromatography over silica gel (eluent: petroleum ether:ethyl acetate=3:1) to give the title compound 2-(1,4-oxazepan-4-yl)acetonitrile (880 mg, 79%) as a yellow oil. LCMS (ESI): mass calcd. for C₇H₁₂N₂O, 140.18; m/z found, 141.2 [M+H]⁺. ¹H NMR (400 MHz, CDCl₃) δ 3.80 (t, J=6.1 Hz, 2H), 3.76-3.69 (m, 2H), 3.58 (s, 2H), 2.84-2.73 (m, 4H), 1.94 (q, J=5.9 Hz, 2H).

### Step b: 2-(1,4-oxazepan-4-yl)ethanamine

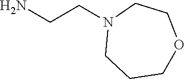

To a solution of 2-(1,4-oxazepan-4-yl)acetonitrile (880 mg, 6.3 mmol) in THF (20 mL) was added lithium aluminium hydride (477 mg, 12.6 mmol) by portions at 0° C. (ice/water), and the resulting mixture was stirred for 1.5 h at 25° C. After cooled to 0° C., the reaction mixture was quenched with water (477 mg) and filtered. The filtrate was then concentrated to dryness under reduced pressure to afford the crude product 2-(1,4-oxazepan-4-yl)ethanamine (700 mg, 77%) as a colorless oil. LCMS (ESI): mass calcd. for C₇H₁₆N₂O, 144.2; m/z found, 145.3[M+H]⁺. ¹H NMR (400 MHz, CDCl₃) δ 3.78 (t, J=6.1 Hz, 2H), 3.72-3.67 (m, 2H), 2.77-2.64 (m, 6H), 2.58-2.50 (m, 2H), 1.87 (q, J=5.9 Hz, 2H).

### Step c: Ethyl 6-methyl-5-((1-methyl-6-((1-methyl-1H-pyrazol-4-yl)amino)-1H-pyrazolo[3,4-d]pyrimidin-3-yl)amino)nicotinate

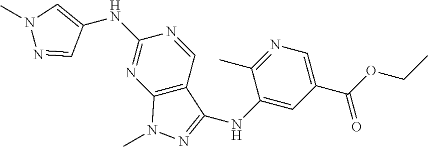 [SMILES: CCOC(=O)c1cnc(C)c(Nc2nn(C)c3nc(Nc4cnn(C)c4)ncc23)c1]

To a solution of ethyl 5-((6-chloro-1-methyl-1H-pyrazolo[3,4-d]pyrimidin-3-yl)amino)-6-methylnicotinate (4 g, 11.5 mmol), 4-amino-1-methylpyrazole (1.34 g, 13.8 mmol), cesium carbonate (11.27 g, 34.6 mmol) and brettphos (1.24 g, 2.31 mmol) in DMF (80 mL) was added brettphos-Pd-G3 (1.04 g, 1.15 mmol) under N₂. The resulting mixture was stirred at 90° C. under N₂ for 12 h before cooled to 25° C. The reaction mixture was diluted with ethyl acetate (50 mL) and filtered over celite. The filtrate was concentrated to the crude product, which was treated with water (70 mL). The residue was filtered and the filter cake was washed with water (50 mL), dried in vacuum to give afford the crude product, which was triturated with methyl tert-butyl ether/ethyl acetate/methanol=5:1:0.2 (120 mL) and filtered to afford ethyl 6-methyl-5-((1-methyl-6-((1-methyl-1H-pyrazol-4-yl)amino)-1H-pyrazolo[3,4-d]pyrimidin-3-yl)amino)nicotinate (3.2 g, 58%) as a black solid. LCMS (ESI): mass calcd. for C₁₉H₂₁N₉O₂, 407.4; m/z found, 408 [M+H]⁺.

### Step d: 6-methyl-5-((1-methyl-6-((1-methyl-1H-pyrazol-4-yl)amino)-1H-pyrazolo[3,4-d]pyrimidin-3-yl)amino)nicotinic acid

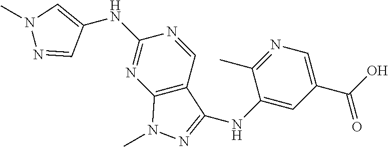 [SMILES: Cc1ncc(C(=O)O)cc1Nc1nn(C)c2nc(Nc3cnn(C)c3)ncc12]

To a solution of ethyl ethyl 6-methyl-5-((1-methyl-6-((1-methyl-1H-pyrazol-4-yl)amino)-1H-pyrazolo[3,4-d]pyrimidin-3-yl)amino)nicotinate (3.4 g, 8.34 mmol) in tetrahydrofuran (20 mL) and water (20 mL) was added lithium hydroxide (1.05 g, 25 mmol) at 25° C. for 2 hours. The mixture was adjusted to pH=3-4 with HCl (aq, 2 M). The mixture was filtered and washed with water (10 mL×3). The solid was evaporated under vacuum to give the desired product 6-methyl-5-((1-methyl-6-((1-methyl-1H-pyrazol-4-yl)amino)-1H-pyrazolo[3,4-d]pyrimidin-3-yl)amino)nicotinic acid (2 g, 63%) as black solid. LCMS (ESI): mass calcd. for C₁₇H₁₇N₉O₂, 379.4; m/z found, 380 [M+H]⁺.

### Step e: N-(2-(1,4-oxazepan-4-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

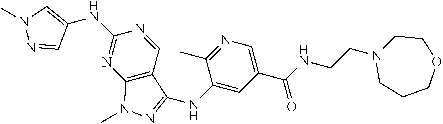 [SMILES: Cc1ncc(C(=O)NCCN2CCCOCC2)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (100 mg, 0.26 mmol), HATU (150 mg, 0.4 mmol) and N,N-diisopropylethylamine (174 ul, 1.05 mmol) in N,N-dimethylformamide (5 mL) was added 2-(1,4-oxazepan-4-yl)ethanamine (49 mg, 0.34 mmol). The mixture stirred at 25° C. for 2 hours and then concentrated under vacuum to give the crude product, which was purified by preparative high-performance liquid chromatography over Column: Welch Xtimate C18 150*30 mm*5 um to give the title compound N-(2-(1,4-oxazepan-4-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (41.6 mg, 30%) as a white solid. LCMS (ESI): mass calcd. for C₂₄H₃₁N₁₁O₂, 505.5; m/z found, 506.4 [M+H]⁺. ¹H NMR (400 MHz, METHANOL-d4) δ 8.85 (s, 1H), 8.76 (d, J=1.8 Hz, 1H), 8.44 (d, J=1.8 Hz, 1H), 8.07 (s, 1H), 7.64 (s, 1H), 3.90 (s, 3H), 3.84 (s, 3H), 3.78 (t, J=6.1 Hz, 2H), 3.76-3.72 (m, 2H), 3.53 (t, J=6.7 Hz, 2H), 2.87-2.75 (m, 6H), 2.64 (s, 3H), 1.91 (td, J=5.9, 11.6 Hz, 2H).

### Example 18. N-(2-(2-oxa-5-azaspiro[3.4]octan-5-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

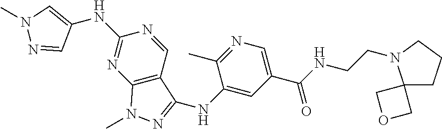 [SMILES: Cc1ncc(C(=O)NCCN2CCCC23COC3)cc1Nc1nn(C)c2nc(Nc3cnn(C)c3)ncc12]

### Step a: 2-(2-oxa-5-azaspiro[3.4]octan-5-yl)acetonitrile

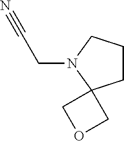 [SMILES: N#CCN1CCCC12COC2]

To a solution of 2-oxa-5-azaspiro[3.4]octane hemioxalate (400 mg, 1.3 mmol) and potassium carbonate (874 mg, 6.3 mmol) in acetonitrile (8 mL) was added 2-bromoacetonitrile (334 mg, 2.8 mmol) at room-temperature. The resulting mixture was stirred at 50° C. for 12 h before cooling to room temperature. The resulting mixture was quenched with water (15 mL) and extracted with ethyl acetate (30 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=3:1) to give the title compound 2-(2-oxa-5-azaspiro[3.4]octan-5-yl)acetonitrile (400 mg, 92%) as a pale yellow oil. ¹H NMR (400 MHz, CDCl₃) δ 4.85 (d, J=7.4 Hz, 2H), 4.54 (d, J=7.3 Hz, 2H), 3.96 (s, 2H), 2.96 (t, J=6.8 Hz, 2H), 2.32-2.18 (m, 2H), 1.91-1.77 (m, 2H).

### Step b: 2-(2-oxa-5-azaspiro[3.4]octan-5-yl)ethanamine

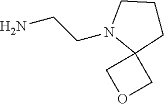 [SMILES: NCCN1CCCC12COC2]

To a solution of 2-(2-oxa-5-azaspiro[3.4]octan-5-yl)acetonitrile (370 mg, 2.4 mmol) in methanol (36 mL) was hydrogenated at room temperature with Raney Ni (14 mg, 0.243 mmol) as a catalyst in the presence of H₂ (15 psi) for 2 hours. After uptake of H₂ (3 eq), the catalyst filtered off and the filtrate was evaporated give the title compound 2-(2-oxa-5-azaspiro[3.4]octan-5-yl)ethanamine (340 mg, 90%) as a pale yellow oil. which was used to the next step without further purification. ¹H NMR (400 MHz, CDCl₃) δ 4.59-4.48 (m, 2H), 2.83-2.69 (m, 2H), 2.60-2.19 (m, 8H), 2.01-1.81 (m, 4H).

### Step c: N-(2-(2-oxa-5-azaspiro[3.4]octan-5-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

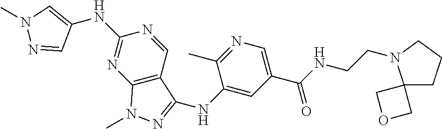 [SMILES: Cc1ncc(C(=O)NCCN2CCCC23COC3)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (140 mg, 0.34 mmol), HATU (261 mg, 0.69 mmol) and N,N-diisopropylethylamine (222 mg, 1.7 mmol) in N,N-dimethylformamide (5 mL) was added 2-(2-oxa-5-azaspiro[3.4]octan-5-yl)ethanamine (80 mg, 0.52 mmol). The mixture stirred at 25° C. for 2 h and then concentrated under vacuum to give the crude product, which was purified by preparative high-performance liquid chromatography over Column: Phenomenex Gemini-NX 80*40 mm*3 um to give the title compound N-(2-(2-oxa-5-azaspiro[3.4]octan-5-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (33 mg, 18%) as an off-white solid. LCMS (ESI): mass calcd. for C₂₅H₃₁N₁₁O₂, 517.3; m/z found, 518.4 [M+H]⁺. ¹H NMR (400 MHz, METHANOL-d₄) δ 8.87 (s, 1H), 8.76 (s, 1H), 8.47 (s, 1H), 8.11 (br s, 1H), 7.67 (s, 1H), 4.82 (br d, J=6.8 Hz, 2H), 4.51 (br d, J=6.7 Hz, 2H), 3.93 (s, 3H), 3.87 (s, 3H), 3.59 (br t, J=6.4 Hz, 2H), 3.14 (br t, J=6.6 Hz, 2H), 2.87 (br t, J=7.0 Hz, 2H), 2.67 (s, 3H), 2.16 (br t, J=7.6 Hz, 2H), 1.79 (q, J=7.3 Hz, 2H).

### Example 19. N-(2-(1-oxa-7-azaspiro[3.5]nonan-7-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

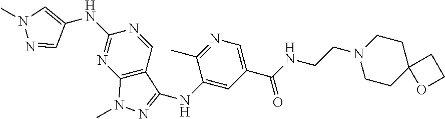 [SMILES: Cc1ncc(C(=O)NCCN2CCC3(CCO3)CC2)cc1Nc1nn(C)c2nc(Nc3cnn(C)c3)ncc12]

### Step a: 2-(1-oxa-7-azaspiro[3.5]nonan-7-yl)acetonitrile

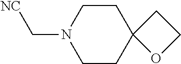 [SMILES: N#CCN1CCC2(CCO2)CC1]

To a solution of 1-oxa-7-azaspiro[3.5]nonane (350 mg, 2.8 mmol) and potassium carbonate (951 mg, 6.9 mmol) in acetonitrile (7 mL) was added 2-bromoacetonitrile (363 mg, 3.1 mmol) at room-temperature. The resulting mixture was stirred at 50° C. for 12 h before cooling to room temperature. The mixture was quenched with water (15 mL) and extracted with ethyl acetate (30 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=3:1) to give the title compound 2-(1-oxa-7-azaspiro[3.5]nonan-7-yl)acetonitrile (368 mg, 80%) as a pale yellow oil. ¹H NMR (400 MHz, CDCl₃) δ 4.46 (t, J=7.8 Hz, 2H), 3.42 (s, 2H), 2.58 (ddd, J=4.2, 7.1, 11.1 Hz, 2H), 2.42 (ddd, J=4.2, 7.2, 11.1 Hz, 2H), 2.33 (t, J=7.8 Hz, 2H), 1.96-1.80 (m, 4H).

### Step b: 2-(1-oxa-7-azaspiro[3.5]nonan-7-yl)ethanamine

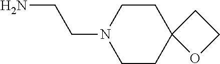 [SMILES: NCCN1CCC2(CCO2)CC1]

To a solution of 2-(1-oxa-7-azaspiro[3.5]nonan-7-yl)acetonitrile (338 mg, 2.0 mmol) in methanol (33 mL) was hydrogenated at room temperature with Raney Ni (12 mg, 0.203 mmol) as a catalyst in the presence of H₂ (15 psi) for 2 hours. After uptake of H₂ (3 eq), the catalyst filtered off and the filtrate was evaporated give the title compound 2-(1-oxa-7-azaspiro[3.5]nonan-7-yl)ethanamine (320 mg, 92%) as a colorless oil which was used to the next step without further purification. ¹H NMR (400 MHz, CDCl₃) δ 4.59-4.48 (m, 2H), 2.83-2.69 (m, 2H), 2.60-2.19 (m, 8H), 2.01-1.81 (m, 4H).

### Step c: N-(2-(1-oxa-7-azaspiro[3.5]nonan-7-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

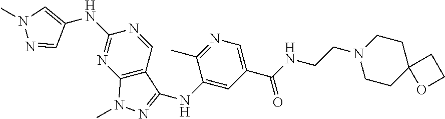 [SMILES: Cc1ncc(C(=O)NCCN2CCC3(CCO3)CC2)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (140 mg, 0.34 mmol), HATU (261 mg, 0.69 mmol) and N,N-diisopropylethylamine (222 mg, 1.7 mmol) in N,N-dimethylformamide (5 mL) was added 2-(1-oxa-7-azaspiro[3.5]nonan-7-yl)ethanamine (117 mg, 0.69 mmol). The mixture stirred at 25° C. for 2 h and then concentrated under vacuum to give the crude product, which was purified by preparative high-performance liquid chromatography over Column: Phenomenex Gemini-NX 80*40 mm*3 um to give the title compound N-(2-(1-oxa-7-azaspiro[3.5]nonan-7-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (30 mg, 16%) as an off-white solid. LCMS (ESI): mass calcd. for C₂₆H₃₃N₁₁O₂, 531.3; m/z found, 532.5 [M+H]⁺. ¹H NMR (400 MHz, METHANOL-d₄) δ 8.87 (s, 1H), 8.75 (s, 1H), 8.47 (s, 1H), 8.10 (br s, 1H), 7.67 (s, 1H), 4.54 (br t, J=7.7 Hz, 2H), 3.92 (s, 3H), 3.87 (s, 3H), 3.56 (br t, J=6.6 Hz, 2H), 2.69-2.57 (m, 7H), 2.55-2.40 (m, 4H), 1.95 (br s, 4H).

### Example 20. N-(2-(2-oxa-7-azaspiro[3.5]nonan-7-yl)ethyl)-6-methyl-5-((1-methyl-6-((1-methyl-1H-pyrazol-4-yl)amino)-1H-pyrazolo[3,4-d] pyrimidin-3-yl)amino)nicotinamide

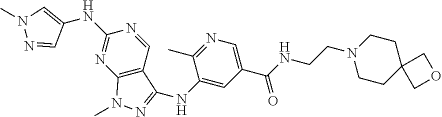 [SMILES: Cc1ncc(C(=O)NCCN2CCC3(CC2)COC3)cc1Nc1nn(C)c2nc(Nc3cnn(C)c3)ncc12]

### Step a: 2-(2-oxa-7-azaspiro[3.5]nonan-7-yl)acetonitrile

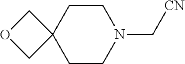 [SMILES: N#CCN1CCC2(CC1)COC2]

To a solution of 2-oxa-7-azaspiro[3.5]nonane (300 mg, 2.4 mmol) and potassium carbonate (815 mg, 5.9 mmol) in acetonitrile (6 mL) was added 2-bromoacetonitrile (311 mg, 2.6 mmol) at room-temperature. The resulting mixture was stirred at 50° C. for 12 h before cooling to room temperature. The mixture was quenched with water (15 mL) and extracted with ethyl acetate (30 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=3:1) to give the title compound 2-(2-oxa-7-azaspiro[3.5]nonan-7-yl)acetonitrile (300 mg, 66%) as a pale yellow oil. ¹H NMR (400 MHz, CDCl₃) δ 4.36 (s, 4H), 3.44 (s, 2H), 2.40 (br t, J=5.2 Hz, 4H), 1.86 (br t, J=5.4 Hz, 4H).

### Step b: 2-(2-oxa-7-azaspiro[3.5]nonan-7-yl)ethanamine

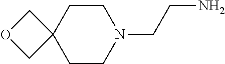 [SMILES: NCCN1CCC2(CC1)COC2]

To a solution of 2-(2-oxa-7-azaspiro[3.5]nonan-7-yl)acetonitrile (270 mg, 1.6 mmol) in methanol (27 mL) was hydrogenated at room temperature with Raney Ni (10 mg, 0.16 mmol) as a catalyst in the presence of H₂ (15 psi) for 2 hours. After uptake of H₂ (3 eq), the catalyst filtered off and the filtrate was evaporated give the title compound 2-(2-oxa-7-azaspiro[3.5]nonan-7-yl)ethanamine (275 mg, 99%) as a colorless oil which was used to the next step without further purification. ¹H NMR (400 MHz, CDCl₃) δ 4.34 (s, 4H), 2.70 (br t, J=6.1 Hz, 2H), 2.28 (br t, J=6.1 Hz, 6H), 1.80 (br s, 4H).

### Step c: N-(2-(2-oxa-7-azaspiro[3.5]nonan-7-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

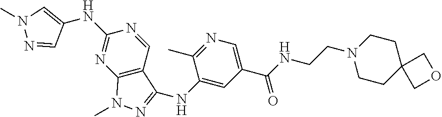 [SMILES: Cc1ncc(C(=O)NCCN2CCC3(CC2)COC3)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (120 mg, 0.29 mmol), HATU (201 mg, 0.53 mmol) and N,N-diisopropylethylamine (190 mg, 1.5 mmol) in N,N-dimethylformamide (5 mL) was added 2-(2-oxa-7-azaspiro[3.5]nonan-7-yl)ethanamine (75 mg, 0.44 mmol). The mixture stirred at 25° C. for 2 h and then concentrated under vacuum to give the crude product, which was purified by preparative high-performance liquid chromatography over Column: Boston Prime C18 150*30 mm*5 um to give the title compound N-(2-(2-oxa-7-azaspiro[3.5]nonan-7-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (45 mg, 18%) as a pale brown solid. LCMS (ESI): mass calcd. for C₂₆H₃₃N₁₁O₂, 531.3; m/z found, 532.3 [M+H]⁺. ¹H NMR (400 MHz, METHANOL-d₄) δ 8.86 (s, 1H), 8.75 (d, J=1.9 Hz, 1H), 8.46 (d, J=1.9 Hz, 1H), 8.10 (s, 1H), 7.67 (s, 1H), 4.43 (s, 4H), 3.92 (s, 3H), 3.87 (s, 3H), 3.56 (t, J=6.8 Hz, 2H), 2.66 (s, 3H), 2.60 (t, J=6.8 Hz, 2H), 2.56-2.35 (m, 1H), 2.48 (br s, 3H), 1.91 (br s, 4H).

### Example 21. 6-methyl-N-(2-(methyl(tetrahydro-2H-pyran-4-yl)amino)ethyl)-5-((1-methyl-6-((1-methyl-1H-pyrazol-4-yl)amino)-1H-pyrazolo[3,4-d]pyrimidin-3-yl)amino)nicotinamide

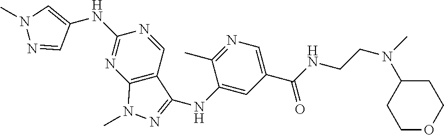 [SMILES: Cc1ncc(C(=O)NCCN(C)C2CCOCC2)cc1Nc1nn(C)c2nc(Nc3cnn(C)c3)ncc12]

### Step a: 2-(methyl(tetrahydro-2H-pyran-4-yl)amino)acetonitrile

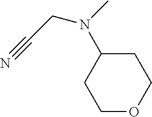

To a solution of N-methyltetrahydro-2H-pyran-4-amine (300 mg, 2.6 mmol) and potassium carbonate (900 mg, 6.5 mmol) in acetonitrile (6 mL) was added 2-bromoacetonitrile (344 mg, 2.9 mmol) at room-temperature. The resulting mixture was stirred at 50° C. for 12 h before cooling to room temperature. The resulting mixture was quenched with water (15 mL) and extracted with ethyl acetate (30 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=3:1) to give the title compound 2-(methyl(tetrahydro-2H-pyran-4-yl)amino)acetonitrile (300 mg, 94%) as a pale yellow oil. ¹H NMR (400 MHz, CDCl₃) δ 4.00-3.91 (m, 2H), 3.55 (s, 2H), 3.34 (dt, J=2.0, 11.9 Hz, 2H), 2.45 (tt, J=4.1, 11.0 Hz, 1H), 2.35 (s, 3H), 1.81-1.72 (m, 2H), 1.50-1.36 (m, 2H).

### Step b: N¹-methyl-N¹-(tetrahydro-2H-pyran-4-yl)ethane-1,2-diamine

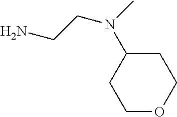

To a solution of 2-(methyl(tetrahydro-2H-pyran-4-yl)amino)acetonitrile (150 mg, 0.97 mmol) in THE (6 mL) was added lithium aluminium hydride (66 mg, 1.8 mmol) by portions at 0° C. (ice/water). The resulting mixture stirred at 20° C. for 1 h before quenched with water (0.2 mL) at 0° C. The reaction mixture was filtered. And the filtrate was concentrated to dryness under reduced pressure to afford the crude product N¹-methyl-N¹-(tetrahydro-2H-pyran-4-yl)ethane-1,2-diamine as a colorless oil, which was used to the next step without further purification. ¹H NMR (400 MHz, CDCl₃) δ 3.95 (br dd, J=4.4, 11.3 Hz, 2H), 3.30 (dt, J=2.3, 11.7 Hz, 2H), 2.68 (t, J=6.1 Hz, 2H), 2.54-2.46 (m, 1H), 2.43 (t, J=6.1 Hz, 2H), 2.19 (s, 3H), 1.64-1.53 (m, 4H).

### Step c: 6-methyl-N-(2-(methyl(tetrahydro-2H-pyran-4-yl)amino)ethyl)-5-((1-methyl-6-((1-methyl-1H-pyrazol-4-yl)amino)-1H-pyrazolo[3,4-d]pyrimidin-3-yl)amino)nicotinamide

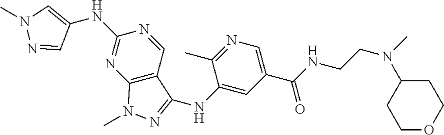 [SMILES: Cc1ncc(C(=O)NCCN(C)C2CCOCC2)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (120 mg, 0.29 mmol), HATU (201 mg, 0.53 mmol) and N,N-diisopropylethylamine (190 mg, 1.5 mmol) in N,N-dimethylformamide (5 mL) was added 2-(2-oxa-7-azaspiro[3.5]nonan-7-yl)ethanamine (70 mg, 0.44 mmol). The mixture stirred at 25° C. for 2 h and then concentrated under vacuum to give the crude product, which was purified by preparative high-performance liquid chromatography over Column: Phenomenex Gemini-NX C18 75*30 mm*3 um to give the title compound 6-methyl-N-(2-(methyl(tetrahydro-2H-pyran-4-yl)amino)ethyl)-5-((1-methyl-6-((1-methyl-1H-pyrazol-4-yl)amino)-1H-pyrazolo[3,4-d]pyrimidin-3-yl)amino) nicotinamide (45 mg, 27%) as a pale brown solid. LCMS (ESI): mass calcd. for C₂₅H₃₃N₁₁O₂, 519.3; m/z found, 520.3 [M+H]⁺. ¹H NMR (400 MHz, METHANOL-d₄) δ 8.86 (s, 1H), 8.76 (d, J=1.7 Hz, 1H), 8.46 (d, J=1.7 Hz, 1H), 8.09 (s, 1H), 7.66 (s, 1H), 3.99 (br dd, J=3.9, 11.1 Hz, 2H), 3.92 (s, 3H), 3.86 (s, 3H), 3.53 (br t, J=6.8 Hz, 2H), 3.40 (br t, J=11.3 Hz, 2H), 2.75 (br t, J=6.8 Hz, 2H), 2.72-2.59 (m, 4H), 2.39 (s, 3H), 1.79 (br d, J=11.0 Hz, 2H), 1.64-1.51 (m, 2H).

### Example 22. N-(2-(4-methoxypiperidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

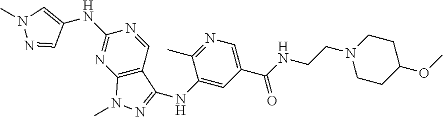

### Step a: tert-butyl (2-(4-methoxypiperidin-1-yl)ethyl)carbamate

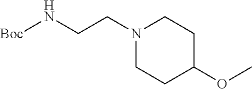 [SMILES: COC1CCN(CCNC(=O)OC(C)(C)C)CC1]

To a solution of tert-butyl (2-bromoethyl)carbamate (0.97 g, 4.3 mmol) and potassium carbonate (1.2 g, 8.7 mmol) in acetonitrile (5 mL) was added 4-methoxypiperidine (500 mg, 4.3 mmol) at room temperature. The resulting mixture was stirred at 50° C. for 16 hrs before cooling to room temperature and filtered. The filtrate was concentrated under reduced pressure to give crude product, which was purified by column chromatography over silica gel (eluent: ethyl acetate/MeOH=5/1, iodine) to give the title compound tert-butyl (2-(4-methoxypiperidin-1-yl)ethyl)carbamate (1.0 g, 89%) as light yellow oil. ¹H NMR (400 MHz, CHLOROFORM-d) δ 4.49-4.43 (m, 1H), 3.69-3.59 (m, 1H), 3.33 (s, 3H), 3.23-3.18 (m, 2H), 2.72 (br s, 2H), 2.42 (br t, J=6.0 Hz, 2H), 2.13 (br t, J=9.7 Hz, 2H), 1.89 (br d, J=11.7 Hz, 2H), 1.73 (s, 2H), 1.44 (s, 9H).

### Step b: 2-(4-methoxypiperidin-1-yl)ethanamine

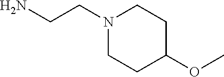

To a solution of tert-butyl (2-(4-methoxypiperidin-1-yl)ethyl)carbamate (1.0 g, 3.9 mmol) in DCM (2 mL) was added HCl/MeOH (10 mL) at room temperature for 2 hrs. The reaction mixture was concentrated in vacuum to give the crude product 2-(4-methoxypiperidin-1-yl)ethanamine (800 mg, 89%) as light yellow oil. ¹H NMR (400 MHz, METHANOL-d₄) δ 4.46-4.38 (m, 1H), 3.68 (br d, J=12.6 Hz, 1H), 3.63-3.54 (m, 2H), 3.45 (br d, J=3.7 Hz, 4H), 3.36-3.34 (m, 2H), 3.33 (s, 3H), 3.29 (td, J=1.7, 3.2 Hz, 1H), 2.26 (br d, J=13.5 Hz, 1H), 2.16-2.02 (m, 2H), 1.86-1.73 (m, 1H).

### Step c: N-(2-(4-methoxypiperidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

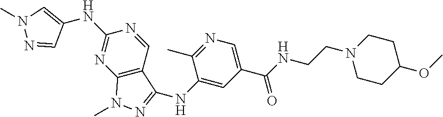

To a solution of 6-methyl-5-((1-methyl-6-((1-methyl-1H-pyrazol-4-yl)amino)-1H-pyrazolo[3,4-d]pyrimidin-3-yl)amino)nicotinic acid (140 mg, 0.37 mmol), 2-(3H-[1,2,3]triazolo[4,5-b]pyridin-3-yl)-1,1,3,3-tetramethylisouronium hexafluorophosphate(V) (210 mg, 0.55 mmol) and N-ethyl-N-isopropylpropan-2-amine (0.24 mL, 1.5 mmol) in DMF (4 mL) was added 2-(4-methoxypiperidin-1-yl)ethanamine (111 mg, 0.48 mmol). The mixture was stirred at 50° C. for 2 hours. The reaction mixture was concentrated under vacuum to give the crude product, which was purified by preparative high-performance liquid chromatography over Column: Phenomenex Gemini-NX C18 75*30 mm*3 um to give the title compound N-(2-(4-methoxypiperidin-1-yl)ethyl)-6-methyl-5-((1-methyl-6-((1-methyl-1H-pyrazol-4-yl)amino)-1H-pyrazolo[3,4-d]pyrimidin-3-yl)amino)-nicotinamide (45.3 mg, 22%) as white solid. LCMS (ESI): mass calcd. for C₂₅H₃₃N₁₁O₂, 519.6; m/z found, 520.3 [M+H]⁺. ¹H NMR (400 MHz, METHANOL-d₄) δ 8.86 (s, 1H), 8.74 (s, 1H), 8.46 (s, 1H), 8.09 (br s, 1H), 7.65 (s, 1H), 3.91 (s, 3H), 3.85 (s, 3H), 3.55 (br t, J=6.7 Hz, 2H), 3.33 (br s, 3H), 3.00 (br s, 1H), 2.84 (br s, 2H), 2.64 (s, 3H), 2.63-2.57 (m, 2H), 2.30 (br s, 2H), 1.92 (br s, 2H), 1.59 (br d, J=9.5 Hz, 2H).

### Example 23. N-(2-(1,1-dioxidothiomorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide

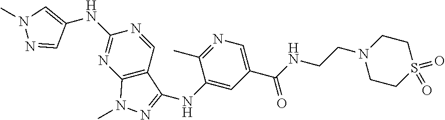 [SMILES: Cc1ncc(C(=O)NCCN2CCS(=O)(=O)CC2)cc1Nc1nn(C)c2nc(Nc3cnn(C)c3)ncc12]

### Step a: N-(2-(1,1-dioxidothiomorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide

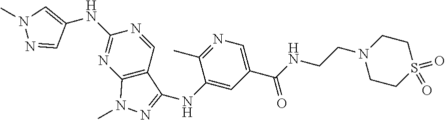 [SMILES: Cc1ncc(C(=O)NCCN2CCS(=O)(=O)CC2)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (120 mg, 0.32 mmol), 2-(3H-[1,2,3]triazolo[4,5-b]pyridin-3-yl)-1,1,3,3-tetramethylisouronium hexafluorophosphate(V) (180 mg, 0.47 mmol) and N-ethyl-N-isopropylpropan-2-amine (209 uL, 1.3 mmol) in DMF (4 mL) was added 4-(2-aminoethyl)thiomorpholine 1,1-dioxide (73 mg, 0.41 mmol). The mixture was stirred at 50° C. for 2 hours. The reaction mixture was concentrated under vacuum to give the crude product, which was purified by preparative high-performance liquid chromatography over Column: Boston Prime C18 150*30 mm*5 um to give pure product. Then the pure product was purified by preparative high-performance liquid chromatography over Column: Phenomenex Gemini-NX 80*40 mm*3 um to give the title compound N-(2-(1,1-dioxidothiomorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide (34.6 mg, 19%) as light brown solid. LCMS (ESI): mass calcd. for C₂₃H₂₉N₁₁O₃S, 539.6; m/z found, 540.40 [M+H]⁺. ¹H NMR (400 MHz, DMSO-d₆) δ 9.76 (br s, 1H), 8.93 (s, 1H), 8.64 (d, J=1.3 Hz, 1H), 8.50-8.46 (m, 2H), 8.39 (s, 1H), 8.08 (br s, 1H), 7.55 (s, 1H), 3.84 (s, 3H), 3.80 (br s, 3H), 3.38 (br s, 2H), 3.07 (br d, J=5.1 Hz, 4H), 2.97 (br d, J=5.3 Hz, 4H), 2.67 (t, J=6.6 Hz, 2H), 2.59 (s, 3H).

### Example 24. N-(2-(7-oxa-4-azaspiro[2.5]octan-4-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

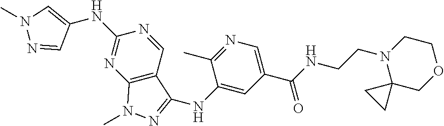 [SMILES: Cc1ncc(C(=O)NCCN2CCOCC23CC3)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (100 mg, 0.26 mmol), HATU (150 mg, 0.4 mmol) and N,N-diisopropylethylamine (174 ul, 1.05 mmol) in N,N-dimethylformamide (5 mL) was added 2-(4-azaspiro[2.4]heptan-4-yl)ethan-1-amine (54 mg, 0.34 mmol). The mixture stirred at 25° C. for 2 hours and then concentrated under vacuum to give the crude product, which was purified by preparative high-performance liquid chromatography over Column: Phenomenex Gemini-NX 80*40 mm*3 um to give the title compound N-(2-(7-oxa-4-azaspiro[2.5]octan-4-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (21.6 mg, 16%) as a white solid. LCMS (ESI): mass calcd. for C₂₅H₃₁N₁₁O₂, 517.6; m/z found, 518.3 [M+H]⁺. ¹H NMR (400 MHz, METHANOL-d₄) δ 8.86 (s, 1H), 8.71 (d, J=1.8 Hz, 1H), 8.42 (d, J=1.8 Hz, 1H), 8.12-8.04 (m, 1H), 7.65 (s, 1H), 3.91 (s, 3H), 3.88-3.83 (m, 3H), 3.77-3.70 (m, 2H), 3.46 (s, 2H), 3.40 (t, J=6.7 Hz, 2H), 3.03-2.95 (m, 4H), 2.65 (s, 3H), 0.74-0.65 (m, 2H), 0.56-0.49 (m, 2H).

### Example 25. N-(2-(2-(methoxymethyl)pyrrolidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

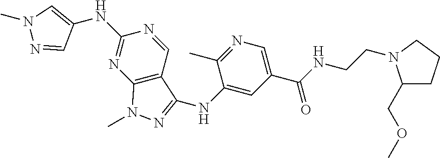 [SMILES: COCC1CCCN1CCNC(=O)c1cnc(C)c(Nc2nn(C)c3nc(Nc4cnn(C)c4)ncc23)c1]

### Step a: tert-butyl (2-(2-(methoxymethyl)pyrrolidin-1-yl)ethyl)carbamate

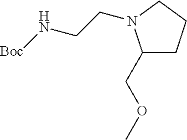 [SMILES: COCC1CCCN1CCNC(=O)OC(C)(C)C]

To a solution of 2-(methoxymethyl)pyrrolidine (170 mg, 1.5 mmol) and potassium carbonate (510 mg, 3.7 mmol) in acetonitrile (6 mL) was added (2-bromo-ethyl)-carbamic acid tert-butyl ester (364 mg, 1.60 mmol) at room-temperature. The resulting mixture was stirred at 50° C. for 12 h before cooling to room temperature. The reaction was concentrated under reduced pressure to give crude product, which was purified by column chromatography over silica gel (eluent: ethyl acetate:methanol=10:1) to give the title compound tert-butyl (2-(2-(methoxymethyl)pyrrolidin-1-yl)ethyl)carbamate (380 mg, 99%) as a pale yellow oil. LCMS (ESI): mass calcd. for C₁₃H₂₆N₂O₃, 258.3; m/z found, 258.9[M+H]⁺. ¹H NMR (400 MHz, CDCl₃) δ 5.20 (br s, 1H), 3.32 (s, 3H), 3.31-3.21 (m, 3H), 3.16-3.01 (m, 2H), 2.97-2.83 (m, 1H), 2.70-2.56 (m, 1H), 2.51-2.37 (m, 1H), 2.20 (q, J=8.3 Hz, 1H), 1.93-1.79 (m, 1H), 1.77-1.64 (m, 2H), 1.61-1.49 (m, 1H), 1.42 (s, 9H).

### Step b: 2-(5-azaspiro[3.4]octan-5-yl)ethanamine

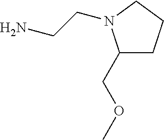

To a solution of tert-butyl (2-(2-(methoxymethyl)pyrrolidin-1-yl)ethyl)carbamate (380 mg, 0.826 mmol) in dichloromethane (5 mL) was added HCl/dioxane (11 mL, 4M) at 0° C. The resulting mixture was stirred at 25° C. for 2 hours. LCMS showed the reaction was completed. Then the reaction mixture was concentrated under reduced pressure to afford the crude product 2-(5-azaspiro[3.4]octan-5-yl)ethanamine as a 2HCl salt white solid. ¹H NMR (400 MHz, METHANOL-d₄) δ 3.90-3.73 (m, 3H), 3.72-3.64 (m, 2H), 3.52-3.45 (m, 1H), 3.43 (s, 3H), 3.42-3.36 (m, 2H), 3.27-3.17 (m, 1H), 2.34-2.22 (m, 1H), 2.21-2.10 (m, 1H), 2.09-1.97 (m, 1H), 1.87-1.75 (m, 1H).

### Step c: N-(2-(2-(methoxymethyl)pyrrolidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

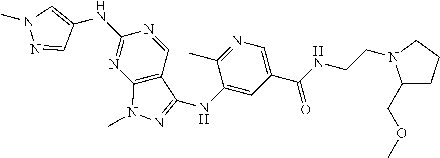 [SMILES: COCC1CCCN1CCNC(=O)c1cnc(C)c(Nc2nn(C)c3nc(Nc4cnn(C)c4)ncc23)c1]

To a solution of 6-methyl-5-((1-methyl-6-((1-methyl-1H-pyrazol-4-yl)amino)-1H-pyrazolo[3,4-d]pyrimidin-3-yl)amino)nicotinic acid (120 mg, 0.32 mmol), HATU (180 mg, 0.47 mmol) and N,N-diisopropylethylamine (209 ul, 1.26 mmol) in N,N-dimethylformamide (5 mL) was added 2-(5-azaspiro[3.4]octan-5-yl)ethanamine 2HCl salt (95 mg, 0.41 mmol). The mixture stirred at 25° C. for 2 hours and then concentrated under vacuum to give the crude product, which was purified by preparative high-performance liquid chromatography over Column: Phenomenex Gemini-NX C18 75*30 mm*3 um to give the title compound N-(2-(2-(methoxymethyl)pyrrolidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (47 mg, 28%) as a white solid. LCMS (ESI): mass calcd. for C₂₅H₃₃N₁₁O₂, 519.6; m/z found, 520.4 [M+H]⁺. ¹H NMR (400 MHz, DMSO-d₆) δ 9.74 (br s, 1H), 8.92 (s, 1H), 8.62 (d, J=1.5 Hz, 1H), 8.46 (d, J=1.7 Hz, 1H), 8.42 (br t, J=5.5 Hz, 1H), 8.37 (br s, 1H), 8.08 (br s, 1H), 7.55 (s, 1H), 3.84 (s, 3H), 3.79 (br s, 3H), 3.23-3.19 (m, 4H), 3.16-3.07 (m, 2H), 2.97 (td, J=7.7, 11.9 Hz, 1H), 2.80 (br s, 1H), 2.64-2.54 (m, 5H), 2.41 (ddd, J=5.2, 7.4, 12.2 Hz, 1H), 2.27-2.16 (m, 1H), 1.87-1.74 (m, 1H), 1.72-1.56 (m, 2H), 1.50-1.39 (m, 1H).

### Example 26. 6-methyl-5-((1-methyl-6-((1-methyl-1H-pyrazol-4-yl)amino)-1H-pyrazolo[3,4-d]pyrimidin-3-yl)amino)-N-(2-(tetrahydro-1H-furo[3,4-c]pyrrol-5(3H)-yl)ethyl)nicotinamide

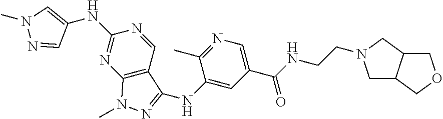 [SMILES: Cc1ncc(C(=O)NCCN2CC3COCC3C2)cc1Nc1nn(C)c2nc(Nc3cnn(C)c3)ncc12]

### Step a: tert-butyl (2-(tetrahydro-1H-furo[3,4-c]pyrrol-5(3H)-yl)ethyl)carbamate

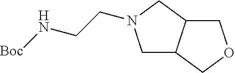 [SMILES: CC(C)(C)OC(=O)NCCN1CC2COCC2C1]

To a solution of Hexahydro-1H-furo[3,4-c]pyrrole (170 mg, 1.5 mmol) and potassium carbonate (519 mg, 3.75 mmol) in acetonitrile (6 mL) was added (2-bromo-ethyl)-carbamic acid tert-butyl ester (370 mg, 1.65 mmol) at room-temperature. The resulting mixture was stirred at 50° C. for 12 h before cooling to room temperature. The reaction was concentrated under reduced pressure to give crude product, which was purified by column chromatography over silica gel (eluent: ethyl acetate:methanol=10:1) to give the title compound tert-butyl (2-(tetrahydro-1H-furo[3,4-c]pyrrol-5(3H)-yl)ethyl)carbamate (300 mg, 78%) as a colorless oil. LCMS (ESI): mass calcd. for C₁₃H₂₄N₂O₃, 256.3; m/z found, 257 [M+H]⁺. ¹H NMR (400 MHz, CDCl₃) δ 4.96 (br s, 1H), 3.81 (br d, J=6.4 Hz, 2H), 3.58-3.45 (m, 2H), 3.21 (br s, 2H), 2.77 (br d, J=2.2 Hz, 2H), 2.64 (br d, J=6.6 Hz, 2H), 2.56-2.46 (m, 2H), 2.36 (br s, 2H), 1.44 (br d, J=8.6 Hz, 9H).

### Step b: 2-(tetrahydro-1H-furo[3,4-c]pyrrol-5(3H)-yl)ethanamine

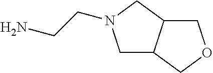 [SMILES: NCCN1CC2COCC2C1]

To a solution of tert-butyl (2-(tetrahydro-1H-furo[3,4-c]pyrrol-5(3H)-yl)ethyl)carbamate (250 mg, 0.98 mmol) in dichloromethane (9 mL) was added trifluoroacetic acid (3 mL, 40.39 mmol) at 0° C. The resulting mixture was stirred at 25° C. for 2 hours. Then the reaction mixture was concentrated under reduced pressure to afford the crude product 2-(tetrahydro-1H-furo[3,4-c]pyrrol-5(3H)-yl)ethanamine as a 2HCl salt white solid. LCMS (ESI): mass calcd. for C₈H₁₆N₂O, 156.2; m/z found, 157.2 [M+H]⁺.

### Step c: 6-methyl-5-((1-methyl-6-((1-methyl-1H-pyrazol-4-yl)amino)-1H-pyrazolo[3,4-d]pyrimidin-3-yl)amino)-N-(2-(tetrahydro-1H-furo[3,4-c]pyrrol-5(3H)-yl)ethyl)nicotinamide

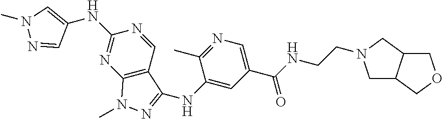 [SMILES: Cc1ncc(C(=O)NCCN2CC3COCC3C2)cc1Nc1nn(C)c2nc(Nc3cnn(C)c3)ncc12]

To a solution of 6-methyl-5-(2-(1-methyl-1H-pyrazol-4-yl)pyrazolo[5,1-b]thiazole-7-carboxamido)nicotinic acid (100 mg, 0.26 mmol), HATU (150 mg, 0.39 mmol) and N,N-diisopropylethylamine (174 ul, 1.05 mmol) in N,N-dimethylformamide (5 mL) was added 2-(tetrahydro-1H-furo[3,4-c]pyrrol-5(3H)-yl)ethanamine 2HCl salt (131 mg, 0.34 mmol). The mixture stirred at 25° C. for 2 h and then concentrated under vacuum to give the crude product, which was purified by preparative high-performance liquid chromatography over Column: Boston Prime C18 150*30 mm*5 um to give the title compound 6-methyl-5-((1-methyl-6-((1-methyl-1H-pyrazol-4-yl)amino)-1H-pyrazolo[3,4-d]pyrimidin-3-yl)amino)-N-(2-(tetrahydro-1H-furo[3,4-c]pyrrol-5(3H)-yl)ethyl)nicotinamide (49.2 mg, 33%) as a yellow solid. LCMS (ESI): mass calcd. for C₂₅H₃₁N₁₁O₂, 517.6; m/z found, 518.3 [M+H]⁺. ¹H NMR (400 MHz, DMSO-d₆) δ 9.41 (br s, 1H), 8.87 (s, 1H), 8.53 (d, J=1.8 Hz, 1H), 8.48 (d, J=1.8 Hz, 1H), 8.17 (br s, 2H), 7.99 (s, 1H), 7.60 (s, 1H), 3.85 (s, 3H), 3.79 (s, 3H), 3.77-3.72 (m, 2H), 3.43-3.36 (m, 4H), 2.71 (br s, 4H), 2.60 (s, 3H), 2.58-2.55 (m, 2H), 2.43 (dd, J=2.8, 9.0 Hz, 2H).

### Example 27. N-(2-(2,2-dimethylpyrrolidin-1-yl)ethyl)-6-methyl-5-((1-methyl-6-(pyrimidin-5-ylamino)-1H-pyrazolo[3,4-d]pyrimidin-3-yl)amino)nicotinamide

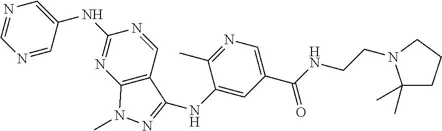 [SMILES: Cc1ncc(C(=O)NCCN2CCCC2(C)C)cc1Nc1nn(C)c2nc(Nc3cncnc3)ncc12]

### Step a: 5-((6-chloro-1-methyl-1H-pyrazolo[3,4-d]pyrimidin-3-yl)amino)-N-(2-(2,2-dimethylpyrrolidin-1-yl)ethyl)-6-methylnicotinamide

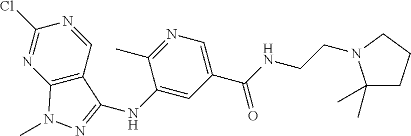 [SMILES: Cc1ncc(C(=O)NCCN2CCCC2(C)C)cc1Nc1nn(C)c2nc(Cl)ncc12]

To a solution of 2-(2,2-dimethylpyrrolidin-1-yl)ethanamine (389 mg, 2.7 mmol) in DCE (4 mL) was added trimethylaluminium (2.0 mol/l in toluene, 1.4 mL, 2.7 mmol) slowly at 0° C. under N₂. The mixture was charged with N₂. The mixture was stirred at 0° C. for 30 min. Then a solution of ethyl 5-((6-chloro-1-methyl-1H-pyrazolo[3,4-d]pyrimidin-3-yl)amino)-6-methylnicotinate (400 mg, 1.1 mmol) in DCE (4 mL) was added slowly. The mixture was stirred at 60° C. for 12 hours. The mixture was cooled to 0° C. Water (2 mL) was added slowly and the mixture was filtered. The filtrate was concentrated to afford to give crude compound. The crude compound was purified by preparative high-performance liquid chromatography over Column: Welch Xtimate C18 100*40 mm*3 um to give the title compound 5-((6-chloro-1-methyl-1H-pyrazolo[3,4-d]pyrimidin-3-yl)amino)-N-(2-(2,2-dimethylpyrrolidin-1-yl)ethyl)-6-methylnicotinamide (127 mg, 26%) as yellow powder. LCMS (ESI): mass calcd. for C₂₁H₂₇ClN₈O, 442.9, m/z found, 443.3 [M+H]⁺. ¹H NMR (400 MHz, DMSO-d₆) δ 9.26 (s, 1H), 8.85 (s, 1H), 8.64 (d, J=1.79 Hz, 1H), 8.56 (d, J=1.91 Hz, 1H), 8.20 (s, 1H), 3.81-3.93 (m, 3H), 3.13-3.40 (m, 2H), 2.88 (br t, J=7.21 Hz, 2H), 2.56-2.66 (m, 5H), 1.66-1.78 (m, 2H), 1.54-1.66 (m, 2H), 0.98 (s, 6H).

### Step b: N-(2-(2,2-dimethylpyrrolidin-1-yl)ethyl)-6-methyl-5-((1-methyl-6-(pyrimidin-5-ylamino)-1H-pyrazolo[3,4-d]pyrimidin-3-yl)amino)nicotinamide

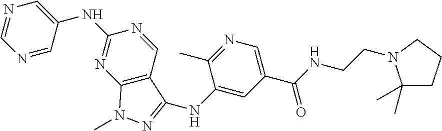 [SMILES: Cc1ncc(C(=O)NCCN2CCCC2(C)C)cc1Nc1nn(C)c2nc(Nc3cncnc3)ncc12]

To a solution of 5-((6-chloro-1-methyl-1H-pyrazolo [3,4-d]pyrimidin-3-yl)amino)-N-(2-(2,2-dimethylpyrrolidin-1-yl)ethyl)-6-methylnicotinamide (100 mg, 0.21 mmol) in DMF (10 mL) was added pyrimidin-5-amine (24.4 mg 0.26 mmol), Then Cs₂CO₃ (209 mg 0.64 mmol), Brettphos (23.0 mg 0.043 mmol) and Brettphos-Pd-G3 (38.8 mg 0.04 mmol) was added. The mixture was charged with N₂. The mixture was stirred at 120° C. for 12 hours. The mixture was cooled to room temperature and filtered. The filtrate was concentrated to afford crude compound. The crude compound was purified by preparative high-performance liquid chromatography over Column: Phenomenex Gemini-NX 80*40 mm*3 um to give the title compound N-(2-(2,2-dimethylpyrrolidin-1-yl)ethyl)-6-methyl-5-((1-methyl-6-(pyrimidin-5-ylamino)-1H-pyrazolo[3,4-d]pyrimidin-3-yl)amino)nicotinamide (34.4 mg, 31%) as yellow powder. LCMS (ESI): mass calcd. for C₂₅H₃₁N₁₁O, 501.5; m/z found, 502.2 [M+H]⁺. ¹H NMR (400 MHz, DMSO-d₆) δ 10.25 (s, 1H), 9.28-9.33 (m, 2H), 9.08-9.13 (m, 1H), 8.78-8.85 (m, 1H), 8.64 (d, J=1.51 Hz, 1H), 8.44-8.56 (m, 3H), 3.81 (s, 3H), 3.25-3.31 (m, 2H), 2.68-2.88 (m, 2H), 2.61 (s, 3H), 2.53-2.57 (m, 2H), 1.64-1.74 (m, 2H), 1.51-1.61 (m, 2H), 0.94 (s, 6H).

### Example 28. N-(2-(2,2-dimethylpyrrolidin-1-yl)ethyl)-6-methyl-5-((1-methyl-6-(pyridin-4-ylamino)-1H-pyrazolo[3,4-d]pyrimidin-3-yl)amino)nicotinamide

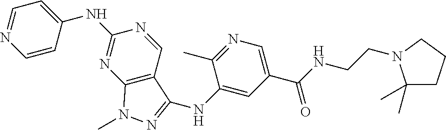 [SMILES: Cc1ncc(C(=O)NCCN2CCCC2(C)C)cc1Nc1nn(C)c2nc(Nc3ccncc3)ncc12]

To a solution of 5-((6-chloro-1-methyl-1H-pyrazolo[3,4-d]pyrimidin-3-yl) amino)-N-(2-(2,2-dimethylpyrroli-din-1-yl)ethyl)-6-methylnicotinamide (71 mg, 0.16 mmol) dissolved in DMF (7 mL) was added pyridin-4-amine (18 mg 0.19 mmol), Then Cs₂CO₃ (157 mg 0.48 mmol), Brettphos (17 mg 0.03 mmol) and Brettphos-Pd-G3 (19 mg 0.03 mmol) was added. The mixture was charged with N₂. The mixture was stirred at 120° C. for 12 hours. The mixture was cooled to room temperature and filtered. The filtrate was concentrated to afford crude compound. The crude compound was purified by preparative high-performance liquid chromatography over Column: Phenomenex Gemini-NX 80*40 mm*3 um to give the title compound N-(2-(2,2-dimethylpyrrolidin-1-yl)ethyl)-6-methyl-5-((1-methyl-6-(pyridin-4-ylamino)-1H-pyrazolo[3,4-d]pyrimidin-3-yl)amino)nicotinamide (9.1 mg, 11%) as brown powder. LCMS (ESI): mass calcd. for C₂₆H₃₂N₁₀O, 500.5; m/z found, 501.1 [M+H]⁺. ¹H NMR (400 MHz, METHANOL-d₄) δ9.01 (s, 1H), 8.78 (d, J=1.71 Hz, 1H), 8.46 (d, J=1.96 Hz, 1H), 8.31-8.35 (m, 2H), 7.89-7.96 (m, 2H), 4.60 (br s, 3H), 3.90 (s, 3H), 1.05 (s, 6H), 3.51 (br t, J=6.85 Hz, 2H), 2.95 (br s, 2H), 2.71 (br s, 2H), 2.65 (s, 3H), 1.79-1.86 (m, 2H), 1.66-1.74 (m, 2H).

### Example 29. 5-((6-((5-carbamoylpyridin-3-yl)amino)-1-methyl-1H-pyrazolo[3, 4-d]pyrimidin-3-yl)amino)-N-(2-(2,2-dimethylpyrrolidin-1-yl)ethyl)-6-methylnicotinamide

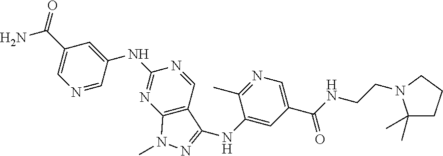 [SMILES: Cc1ncc(C(=O)NCCN2CCCC2(C)C)cc1Nc1nn(C)c2nc(Nc3cncc(C(N)=O)c3)ncc12]

### Step a: methyl 5-aminonicotinate

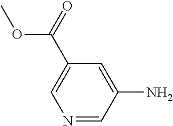

A solution of 5-aminonicotinic acid (500 mg, 3.6 mmol) in HCl (6 mol/L in methanol, 5 mL, 30 mmol) was stirred at 75° C. overnight. The mixture was concentrated to give the crude compound methyl 5-aminonicotinate. The crude compound was used for nest step without purification. ¹H NMR (400 MHz, DMSO-d₆) δ 8.28 (s, 1H), 8.14 (br s, 1H), 7.87-7.96 (m, 1H), 3.79-3.86 (m, 3H).

### Step b: methyl 5-((3-((5-((2-(2,2-dimethylpyrrolidin-1-yl)ethyl)carbamoyl)-2-methyl pyridin-3-yl)amino)-1-methyl-1H-pyrazolo[3,4-d]pyrimidin-6-yl)amino)nicotinate

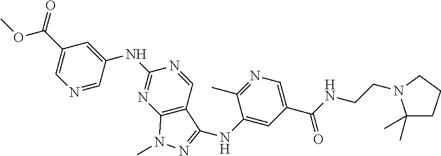 [SMILES: COC(=O)c1cncc(Nc2ncc3c(Nc4cc(C(=O)NCCN5CCCC5(C)C)cnc4C)nn(C)c3n2)c1]

To a solution of 5-((6-chloro-1-methyl-1H-pyrazolo [3,4-d]pyrimidin-3-yl)amino)-N-(2-(2,2-dimethylpyrroli-din-1-yl)ethyl)-6-methylnicotinamide (200 mg, 0.45 mmol) dissolved in DMF (30 mL) was added methyl 5-aminonicotinate (82 mg 0.54 mmol), Then Cs₂CO₃ (436 mg 1.3 mmol), Brettphos (48 mg 0.09 mmol) and Brettphos-Pd-G3 (81 mg 0.09 mmol) was added. The mixture was charged with N₂. The mixture was stirred at 120° C. for 12 hours. The mixture was cooled to room temperature and filtered. The filtrate was concentrated to afford crude compound. The crude compound was purified by preparative high-performance liquid chromatography over Column: Xtimate C18 150*40 mm*5 um to give the title compound methyl 5-((3-((5-((2-(2,2-dimethylpyrrolidin-1-yl) ethyl) carbamoyl)-2-methylpyridin-3-yl) amino)-1-methyl-1H-pyrazolo [3,4-d]pyrimidin-6-yl)amino)nicotinate (79.3 mg, 30%) as a yellow powder. LCMS (ESI): mass calcd. for C₂₈H₃₄N₁₀O₃, 558.6; m/z found, 559.3. [M+H]⁺.

### Step c: 5-((6-((5-carbamoylpyridin-3-yl)amino)-1-methyl-1H-pyrazolo[3,4-d]pyrimidin-3-yl)amino)-N-(2-(2,2-dimethylpyrrolidin-1-yl)ethyl)-6-methylnicotinamide

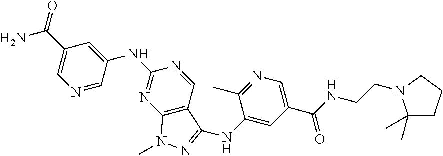 [SMILES: Cc1ncc(C(=O)NCCN2CCCC2(C)C)cc1Nc1nn(C)c2nc(Nc3cncc(C(N)=O)c3)ncc12]

To a solution methyl 5-((3-((5-((2-(2,2-dimethylpyrrolidin-1-yl)ethyl) carbamoyl)-2-methylpyridin-3-yl) amino)-1-methyl-1H-pyrazolo[3,4-d]pyrimidin-6-yl)amino)nicotinate (65 mg, 0.11 mmol) dissolved in NH₃ (7 mol/L in methanol, 9.1 mL, 63.7 mmol)) was added CaCl₂) (12 mg, 0.11 mmol). The mixture was stirred at 70° C. for 3 days. The mixture was cooled to room temperature and filtered. The filtrate was concentrated to afford crude compound. The crude compound was purified by preparative high-performance liquid chromatography over Column: Phenomenex Gemini-NX 80*40 mm*3 um to give the title compound 5-((6-((5-carbamoylpyridin-3-yl)amino)-1-methyl-1H-pyrazolo[3,4-d]pyrimidin-3-yl)amino)-N-(2-(2,2-dimethylpyrrolidin-1-yl)ethyl)-6-methylnicotinamide (17.4 mg, 29%) as white powder. LCMS (ESI): mass calcd. for C₂₇H₃₃N₁₁O₃, 543.6; m/z found, 544.2. [M+H]⁺. ¹H NMR (400 MHz, DMSO-d₆) δ 10.23 (s, 1H), 9.08-9.13 (m, 2H), 8.82 (s, 1H), 8.66 (br d, J=3.76 Hz, 2H), 8.51 (br d, J=5.77 Hz, 2H), 8.14 (br s, 2H), 7.56 (br s, 1H), 3.81 (s, 5H), 2.68 (br s, 3H), 2.62 (s, 4H), 1.52-1.80 (m, 4H), 0.82-1.12 (m, 6H).

### Example 30. 5-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)nicotinic acid

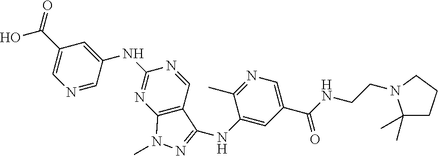 [SMILES: Cc1ncc(C(=O)NCCN2CCCC2(C)C)cc1Nc1nn(C)c2nc(Nc3cncc(C(=O)O)c3)ncc12]

To a solution methyl 5-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)nicotinate (65 mg, 0.11 mmol) dissolved in NH₃ (7 mol/L in methanol, 9.1 mL, 63.7 mmol) was added CaCl₂ (12 mg, 0.11 mmol). The mixture was stirred at 70° C. for 3 days. The mixture was cooled to room temperature and filtered. The filtrate was concentrated to afford crude compound. The crude compound was purified by preparative high-performance liquid chromatography over Column: Phenomenex Gemini-NX 80*40 mm*3 um to give the title compound 5-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)nicotinic acid (18.8 mg, 28%) as a white powder. LCMS (ESI): mass calcd. for C₂₇H₃₂N₁₀O₃, 544.6; m/z found, 545.2. [M+H]⁺. ¹H NMR (400 MHz, METHANOL-d₄) δ 9.05 (s, 1H), 8.98 (br s, 1H), 8.85 (s, 1H), 8.71 (br s, 1H), 8.46 (br d, J=15.26 Hz, 2H), 8.26 (br s, 1H), 3.83-3.90 (m, 5H), 3.37-3.45 (m, 3H), 2.60 (s, 3H), 2.10-2.19 (m, 4H), 1.46 (br s, 6H).

### Example 31. N-(2-(2,2-dimethylpyrrolidin-1-yl)ethyl)-6-methyl-5-((1-methyl-6-((1-methyl-6-oxo-1,6-dihydropyridazin-3-yl)amino)-1H-pyrazolo[3,4-d]pyrimidin-3-yl) amino)nicotinamide

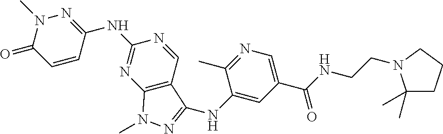 [SMILES: Cc1ncc(C(=O)NCCN2CCCC2(C)C)cc1Nc1nn(C)c2nc(Nc3ccc(=O)n(C)n3)ncc12]

### Step a: tert-butyl (1-methyl-6-oxo-1,6-dihydropyridazin-3-yl)carbamate

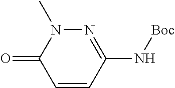 [SMILES: Cn1nc(NC(=O)OC(C)(C)C)ccc1=O]

To a solution of 6-chloro-2-methylpyridazin-3(2H)-one (500 mg, 3.5 mmol), tert-butyl carbamate (608 mg, 5.2 mmol) and Cs₂CO₃ (3.4 g, 10.4 mmol) in dioxane (50 mL) was added X-phos (165 mg, 0.35 mmol) and Pd₂(dba)₃ (317 mg, 0.35 mmol) under N₂. The mixture was stirred at 90° C. overnight under N₂. The mixture was cooled to room temperature and filtered. The filtrate was diluted with ethyl acetate (80 mL) and washed with water (80 mL*3). The organic layer was dried over MgSO₄, filtered and evaporated to afford crude product. The crude product was purified by column chromatography over silica gel (eluent: petroleum ether/ethyl acetate=100:0-0:100). The fractions were collected and the solvent was removed in vacuum to give title compound tert-butyl (1-methyl-6-oxo-1,6-dihydropyridazin-3-yl)carbamate (663 mg, 68%) as a yellow solid. LCMS (ESI): mass calcd. for C₁₀H₁₅N₃O₃, 225.2; m/z found, 226.2 [M+H]⁺.

### Step b: 6-amino-2-methylpyridazin-3(2H)-one

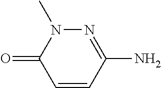

A solution of tert-butyl (1-methyl-6-oxo-1,6-dihydropyridazin-3-yl)carbamate (600 mg, 2.2 mmol) in HCl/dioxane (20 mL, 4M, 80 mmol) was stirred at room temperature for 1 hour. The mixture was evaporated to afford crude compound 6-amino-2-methylpyridazin-3(2H)-one (374 mg, 95%) as a yellow oil. LCMS (ESI): mass calcd. for C₅H₇N₃O, 125.1; m/z found, 126.2 [M+H]⁺.

### Step c: N-(2-(2,2-dimethylpyrrolidin-1-yl)ethyl)-6-methyl-5-((1-methyl-6-((1-methyl-6-oxo-1,6-dihydropyridazin-3-yl)amino)-1H-pyrazolo[3,4-d]pyrimidin-3-yl)amino)nicotinamide

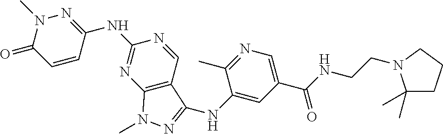 [SMILES: Cc1ncc(C(=O)NCCN2CCCC2(C)C)cc1Nc1nn(C)c2nc(Nc3ccc(=O)n(C)n3)ncc12]

To a solution of 5-((6-chloro-1-methyl-1H-pyrazolo [3,4-d]pyrimidin-3-yl)amino)-N-(2-(2,2-dimethyl-pyrrolidin-1-yl)ethyl)-6-methylnicotinamide (150 mg, 0.34 mmol) in DMF (10 mL) was added 6-amino-2-methylpyridazin-3(2H)-one (90 mg, 0.51 mmol), Then Cs₂CO₃ (331 mg 1.02 mmol), Brettphos (36 mg, 0.07 mmol) and Brettphos-Pd-G3 (60 mg, 0.07 mmol) was added. The mixture was charged with N₂. The mixture was stirred at 120° C. for 12 hours. The mixture was cooled to room temperature and filtered. The filtrate was concentrated to afford crude compound. The crude compound was purified by preparative high-performance liquid chromatography over Column: Xtimate C18 150*40 mm*5 um to give the title compound N-(2-(2,2-dimethylpyrrolidin-1-yl)ethyl)-6-methyl-5-((1-methyl-6-((1-methyl-6-oxo-1,6-dihydropyridazin-3-yl)amino)-1H-pyrazolo[3,4-d]pyrimidin-3-yl)amino)nicotinamide (59.3 mg, 32%) as a yellow powder. LCMS (ESI): mass calcd. for C₂₆H₃₃N₁₁O₂, 531.6; m/z found, 532.4 [M+H]⁺. ¹H NMR (400 MHz, METHANOL-d₄) δ 8.88 (s, 1H), 8.74 (d, J=1.91 Hz, 1H), 8.40-8.45 (m, 2H), 6.93 (d, J=9.78 Hz, 1H), 3.73 (s, 3H), 3.60-3.66 (m, 5H), 3.36 (br d, J=17.17 Hz, 2H), 3.05 (br s, 2H), 2.57 (s, 3H), 1.85-1.99 (m, 4H), 1.21 (br s, 6H).

### Example 32. 5-((6-((6-aminopyridin-3-yl)amino)-1-methyl-1H-pyrazolo[3,4-d]pyrimidin-3-yl)amino)-N-(2-(2,2-dimethylpyrrolidin-1-yl)ethyl)-6-methylnicotinamide

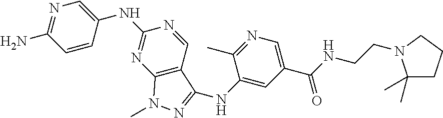 [SMILES: Cc1ncc(C(=O)NCCN2CCCC2(C)C)cc1Nc1nn(C)c2nc(Nc3ccc(N)nc3)ncc12]

### Step a: tert-butyl (5-((3-((5-((2-(2,2-dimethylpyrrolidin-1-yl)ethyl)carbamoyl)-2-methyl-pyri-din-3-yl)amino)-1-methyl-1H-pyrazolo[3,4-d]pyrimidin-6-yl)amino)pyridin-2-yl) carbamate

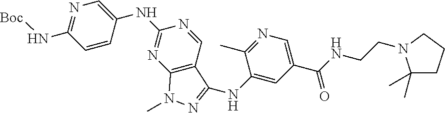 [SMILES: Cc1ncc(C(=O)NCCN2CCCC2(C)C)cc1Nc1nn(C)c2nc(Nc3ccc(NC(=O)OC(C)(C)C)nc3)ncc12]

To a solution of 5-((6-chloro-1-methyl-1H-pyrazolo [3,4-d]pyrimidin-3-yl)amino)-N-(2-(2,2-dimethylpyrrolidin-1-yl)ethyl)-6-methylnicotinamide (100 mg, 0.23 mmol) in DMF (10 mL) was added tert-butyl (5-aminopyridin-2-yl)carbamate (71 mg 0.34 mmol), Then Cs₂CO₃ (220 mg 0.68 mmol), Brettphos (24 mg, 0.05 mmol) and Brettphos-Pd-G3 (41 mg, 0.05 mmol) was added. The mixture was charged with N₂. The mixture was stirred at 90° C. for 12 hours. The mixture was cooled to room temperature and filtered. The filtrate was concentrated to afford crude compound. The crude compound was purified by preparative high-performance liquid chromatography over Column: Xtimate C18 150*40 mm*5 um to give the title compound tert-butyl (5-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)pyridin-2-yl)carbamate (75.1 mg, 23%) as a white powder. LCMS (ESI): mass calcd. for C₃₁H₄₁N₁₁O₃, 615.7; m/z found, 616.4 [M+H]⁺.

### Step b: 5-((6-((6-aminopyridin-3-yl)amino)-1-methyl-1H-pyrazolo[3,4-d]pyrimidin-3-yl)amino)-N-(2-(2,2-dimethylpyrrolidin-1-yl)ethyl)-6-methylnicotinamide

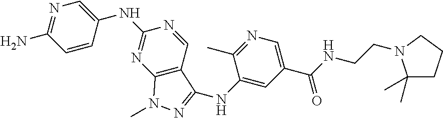 [SMILES: Cc1ncc(C(=O)NCCN2CCCC2(C)C)cc1Nc1nn(C)c2nc(Nc3ccc(N)nc3)ncc12]

A solution of tert-butyl (5-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)pyridin-2-yl)carbamate (70 mg, 0.10 mmol) in HCl/dioxane (2 mL, 4M, 8 mmol) was stirred at room temperature for 1 hour. The mixture was evaporated to afford crude compound. The crude compound was purified by preparative high-performance liquid chromatography over Column: Phenomenex Gemini-NX C18 75*30 mm*3 um to give the title compound 5-((6-((6-aminopyridin-3-yl)amino)-1-methyl-H-pyrazolo[3,4-d]pyrimidin-3-yl)amino)-N-(2-(2,2-dimethyl-pyrrolidin-1-yl)ethyl)-6-methylnicotinamide (17.8 mg, 17%) as a white powder. LCMS (ESI): mass calcd. for C₂₆H₃₃N₁₁O, 515.6; m/z found, 516.3 [M+H]⁺. ¹H NMR (400 MHz, DMSO-d₆) δ 9.39 (br s, 1H), 8.88 (s, 1H), 8.57 (d, J=1.71 Hz, 1H), 8.34-8.44 (m, 3H), 8.25 (br s, 1H), 7.76 (br d, J=7.58 Hz, 1H), 6.43 (d, J=8.80 Hz, 1H), 5.60 (s, 2H) 3.68 (s, 3H), 3.25 (br d, J=6.85 Hz, 2H), 2.69-2.74 (m, 2H), 2.52-2.65 (m, 5H), 1.61-1.66 (m, 2H), 1.45-1.52 (m, 2H), 0.88 (s, 6H).

### Example 33. 2-(5-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)pyridin-3-yl)acetic acid

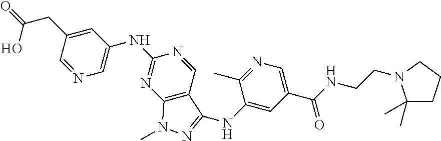 [SMILES: Cc1ncc(C(=O)NCCN2CCCC2(C)C)cc1Nc1nn(C)c2nc(Nc3cncc(CC(=O)O)c3)ncc12]

### Step a: methyl 2-(5-bromopyridin-3-yl)acetate

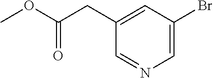

A solution of 2-(5-bromopyridin-3-yl)acetic acid (500 mg, 2.31 mmol) in methanol/HCl (5 mL, 4 M, 20 mmol) was stirred at room temperature for 2 hours. The mixture was evaporated in vacuum to give crude compound methyl 2-(5-bromopyridin-3-yl)acetate as white solid. The crude compound was used for nest step without purification (500 mg, 99%). LCMS (ESI): mass calcd. for C₈H₈BrNO₂, 230.1; m/z found, 232.0 [M+2]⁺.

### Step b: methyl 2-(5-((tert-butoxycarbonyl)amino)pyridin-3-yl)acetate

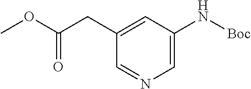 [SMILES: COC(=O)Cc1cncc(NC(=O)OC(C)(C)C)c1]

To a solution of methyl 2-(5-bromopyridin-3-yl)acetate (500 mg, 2.2 mmol), tert-butyl carbamate (380 mg, 3.2 mmol) and Cs₂CO₃ (2.1 g, 6.5 mmol) in dioxane (50 mL) was added X-phos (103 mg, 0.22 mmol) and (dba)₃Pd₂ (198 mg, 0.22 mmol) under N₂. The mixture was stirred at 80° C. overnight under N₂. The mixture was cooled to room temperature and filtered. The filtrate was diluted with ethyl acetate (80 mL) and washed with water (80 mL*3). The organic layer was dried over MgSO₄, filtered, and evaporated to afford crude product. The crude product was purified by column chromatography over silica gel (eluent: petroleum ether/ethyl acetate=100:0-0:100). The fractions were collected, the solvent was removed in vacuum to give crude compound. The crude compound was purified by preparative high-performance liquid chromatography over Column: Xtimate C18 100*30 mm*3 um to give the title compound methyl 2-(5-((tert-butoxycarbonyl)amino)pyridin-3-yl)acetate (162 mg, 33%) as a white solid. LCMS (ESI): mass calcd. for C₁₃H₁₈N₂O₄, 266.3; m/z found, 226.9 [M+H]⁺.

### Step c: methyl 2-(5-aminopyridin-3-yl)acetate

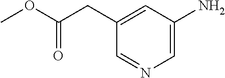

A solution of methyl 2-(5-((tert-butoxycarbonyl)amino)pyridin-3-yl)acetate (162 mg, 0.61 mmol) in HCl/dioxane (6.5 mL, 4 M, 25.9 mmol) was stirred at room temperature for 1 hour. The mixture was evaporated to afford crude compound methyl 2-(5-aminopyridin-3-yl)acetate as yellow oil. The crude compound was used for nest step without purification. LCMS (ESI): mass calcd. for C₈H₁₀N₂O₂, 166.2; m/z found, 167.2 [M+H]⁺.

### Step d: methyl 2-(5-((3-((5-((2-(2,2-dimethylpyrrolidin-1-yl) ethyl) carbamoyl)-2-methylpyridin-3-yl)amino)-1-methyl-1H-pyrazolo[3,4-d]pyrimidin-6-yl)amino)pyridin-3-yl)acetate

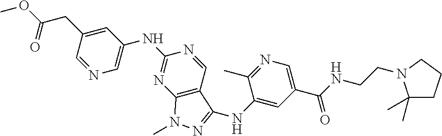 [SMILES: COC(=O)Cc1cncc(Nc2ncc3c(Nc4cc(C(=O)NCCN5CCCC5(C)C)cnc4C)nn(C)c3n2)c1]

To a solution of 5-((6-chloro-1-methyl-1H-pyrazolo [3,4-d]pyrimidin-3-yl)amino)-N-(2-(2,2-dimethylpyrrolidin-1-yl)ethyl)-6-methylnicotinamide (100 mg, 0.23 mmol) in DMF (10 mL) was added tert-butyl (5-aminopyridin-2-yl)carbamate (69 mg 0.34 mmol), Then Cs₂CO₃ (220 mg, 0.68 mmol), Brettphos (24 mg, 0.05 mmol) and Brettphos-Pd-G3 (41 mg, 0.05 mmol) was added. The mixture was charged with N₂. The mixture was stirred at 90° C. for 12 hours. The mixture was cooled to room temperature and filtered. The filtrate was concentrated to afford crude compound. The crude compound was purified by preparative high-performance liquid chromatography over Column: Xtimate C18 150*40 mm*5 um to give the title compound methyl 2-(5-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)pyridin-3-yl)acetate (56.3 mg, 22%) as white powder. LCMS (ESI): mass calcd. for C₂₉H₃₆N₁₀O₃, 572.7; m/z found, 573.2 [M+H]⁺.

### Step e: 2-(5-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)pyridin-3-yl)acetic acid

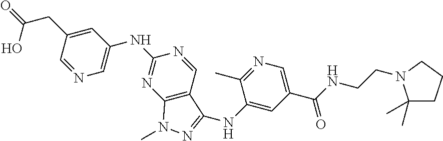 [SMILES: Cc1ncc(C(=O)NCCN2CCCC2(C)C)cc1Nc1nn(C)c2nc(Nc3cncc(CC(=O)O)c3)ncc12]

To a solution of methyl 2-(5-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)pyridin-3-yl)acetate (50 mg, 0.09 mmol) in THF (3 mL) and H₂O (1 mL) was added lithium hydroxide (18 mg, 0.432 mmol) at room temperature. The reaction mixture was stirred at 20° C. for 1.5 h. The mixture was adjusted to pH=3-4 with HCl (aq, 2 M). The mixture was evaporated under vacuum to give the crude compound. The crude compound was purified by preparative high-performance liquid chromatography over Column: Phenomenex Gemini-NX C18 75*30 mm*3 um to give the title compound 2-(5-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)pyridin-3-yl)acetic acid (31.3 mg, 65%) as a yellow powder. LCMS (ESI): mass calcd. for C₂₈H₃₄N₁₀O₃, 558.6; m/z found, 559.3 [M+H]⁺. 1H NMR (400 MHz, DMSO-d₆) δ 10.09 (s, 1H), 9.07 (s, 1H), 8.85 (d, J=2.26 Hz, 1H), 8.68 (d, J=1.76 Hz, 2H), 8.47-8.59 (m, 2H), 8.40 (s, 1H), 8.18 (s, 1H), 8.08 (d, J=1.51 Hz, 1H), 3.80 (s, 3H), 3.63-3.65 (m, 2H), 3.40 (br d, J=6.02 Hz, 2H), 2.96 (br d, J=7.28 Hz, 2H), 2.65-2.72 (m, 2H), 2.61 (s, 3H), 1.76 (br s, 2H), 1.65 (br d, J=8.28 Hz, 2H), 1.03 (s, 6H).

### Example 34. N-(2-(2,2-dimethylpyrrolidin-1-yl)ethyl)-6-methyl-5-((1-methyl-6-((1-methyl-1H-pyrazol-3-yl)amino)-1H-pyrazolo[3,4-d]pyrimidin-3-yl)amino)nicotinamide

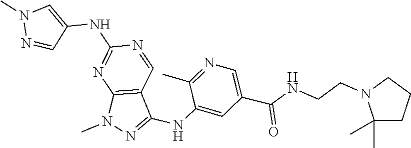 [SMILES: Cc1ncc(C(=O)NCCN2CCCC2(C)C)cc1Nc1nn(C)c2nc(Nc3cnn(C)c3)ncc12]

To a solution of 5-((6-chloro-1-methyl-1H-pyrazolo[3,4-d]pyrimidin-3-yl)amino)-N-(2-(2,2-dimethyl-pyrrolidin-1-yl)ethyl)-6-methylnicotinamide (200 mg, 0.44 mmol) dissolved in DMF (10 mL) was added 1-methyl-1H-pyrazol-3-amine (63 mg, 0.66 mmol), Then Cs₂CO₃ (428 mg, 1.3 mmol), Brettphos (47 mg, 0.09 mmol) and Brettphos-Pd-G3 (79 mg, 0.09 mmol) was added. The mixture was charged with N₂ and then stirred at 80° C. for 12 hours before cooling to room temperature. The reaction was concentrated under reduced pressure to give crude product, which was purified by preparative high-performance liquid chromatography over Column: Prime C18 150*30 mm*5 um to give the title compound N-(2-(2,2-dimethylpyrrolidin-1-yl)ethyl)-6-methyl-5-((1-methyl-6-((1-methyl-1H-pyrazol-3-yl)amino)-1H-pyrazolo[3,4-d]pyrimidin-3-yl)amino)nicotinamide (32.3 mg, 14%) as a yellow solid. LCMS (ESI): mass calcd. for C₂₅H₃₃N₁₁O, 503.6; m/z found, 504.2 [M+H]⁺. ¹H NMR (400 MHz, METHANOL-d₄) δ 8.93 (s, 1H), 8.78 (d, J=1.91 Hz, 1H), 8.48 (d, J=1.91 Hz, 1H), 7.52 (d, J=2.26 Hz, 1H), 6.85 (d, J=2.26 Hz, 1H), 3.86 (s, 3H), 3.85 (s, 3H), 3.49-3.56 (m, 2H), 2.95 (br d, J=2.15 Hz, 2H), 2.68 (s, 5H), 1.79-1.91 (m, 2H), 1.67-1.78 (m, 2H), 1.07 (s, 6H).

### Example 35. N-(2-(2,2-dimethylpyrrolidin-1-yl)ethyl)-6-methyl-5-((1-methyl-6-((6-oxo-1,6-dihydropyridin-3-yl)amino)-1H-pyrazolo[3,4-d]pyrimidin-3-yl)amino)nicotinamide

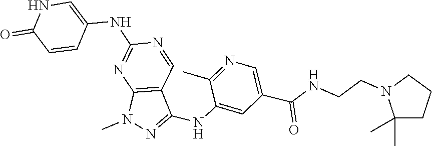 [SMILES: Cc1ncc(C(=O)NCCN2CCCC2(C)C)cc1Nc1nn(C)c2nc(Nc3ccc(=O)[nH]c3)ncc12]

To a solution of 5-((6-chloro-1-methyl-1H-pyrazolo[3,4-d]pyrimidin-3-yl)amino)-N-(2-(2,2-dimethyl-pyrrolidin-1-yl)ethyl)-6-methylnicotinamide (150 mg, 0.34 mmol) dissolved in DMF (15 mL) was added 1-methyl-1H-pyrazol-3-amine (56 mg, 0.51 mmol), Then Cs₂CO₃ (331 mg, 1.0 mmol), Brettphos (36 mg, 0.07 mmol) and Tris(dibenzylideneacetone)dipalladium(0) (62 mg, 0.07 mmol) was added. The mixture was charged with N₂ and then stirred at 80° C. for 12 hours before cooling to room temperature. The reaction was concentrated under reduced pressure to give crude product, which was purified by preparative high-performance liquid chromatography over Column: Boston Prime C18 150*30 mm*5 um to give the title compound N-(2-(2,2-dimethylpyrrolidin-1-yl)ethyl)-6-methyl-5-((1-methyl-6-((6-oxo-1,6-dihydropyridin-3-yl)amino)-1H-pyrazolo[3,4-d]pyrimidin-3-yl)amino)nicotinamide (28.8 mg, 16%) as a light yellow solid. LCMS (ESI): mass calcd. for C₂₆H₃₂N₁₀O₂, 516.6; m/z found, 517.3 [M+H]⁺. ¹H NMR (400 MHz, DMSO-d₆) δ 11.30 (br s, 1H), 9.52 (br s, 1H), 8.96 (s, 1H), 8.59-8.64 (m, 1H), 8.39-8.50 (m, 3H), 8.09 (br s, 1H), 7.71 (dd, J=9.66, 2.62 Hz, 1H), 6.39 (d, J=9.66 Hz, 1H), 3.74 (s, 3H), 2.69-2.87 (m, 2H), 2.52-2.62 (m, 7H), 1.64-1.77 (m, 2H), 1.49-1.63 (m, 2H), 0.94 (s, 6H).

### Example 36. N-(2-(2,2-dimethylpyrrolidin-1-yl)ethyl)-6-methyl-5-((1-methyl-6-((6-oxo-1,6-dihydropyridazin-3-yl)amino)-1H-pyrazolo[3,4-d]pyrimidin-3-yl)amino) nicotinamide

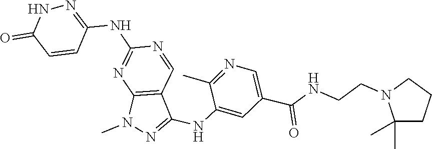 [SMILES: Cc1ncc(C(=O)NCCN2CCCC2(C)C)cc1Nc1nn(C)c2nc(Nc3ccc(=O)[nH]n3)ncc12]

To a solution of 5-((6-chloro-1-methyl-1H-pyrazolo[3,4-d]pyrimidin-3-yl)amino)-N-(2-(2,2-dimethylpyrrolidin-1-yl)ethyl)-6-methylnicotinamide (150 mg, 0.34 mmol) dissolved in DMF (15 mL) was added 6-aminopyridazin-3-ol (56 mg, 0.51 mmol), Then Cs₂CO₃ (331 mg, 1.0 mmol), Brettphos (36 mg, 0.07 mmol) and Brettphos-Pd-G3 (61 mg, 0.07 mmol) was added. The mixture was charged with N₂ and then stirred at 90° C. for 12 hours before cooling to room temperature. The reaction was concentrated under reduced pressure to give crude product, which was purified by preparative high-performance liquid chromatography over Column: Prime C18 150*30 mm*5 um to give the title compound N-(2-(2,2-dimethylpyrrolidin-1-yl)ethyl)-6-methyl-5-((1-methyl-6-((6-oxo-1,6-dihydropyridazin-3-yl)amino)-1H-pyrazolo[3,4-d]pyrimidin-3-yl)amino)nicotinamide (31.3 mg, 17%) as a light yellow solid. LCMS (ESI): mass calcd. for C₂₅H₃₁N₁₁O₂, 517.6; m/z found, 518.4 [M+H]⁺. ¹H NMR (400 MHz, DMSO-d₆) δ ppm 12.60 (br s, 1H), 10.14 (s, 1H), 9.02 (s, 1H), 8.63 (br s, 1H), 8.51 (br d, J=11.32 Hz, 3H), 8.12 (br d, J=10.13 Hz, 1H), 6.93 (br d, J=9.89 Hz, 1H), 3.73 (s, 3H), 2.83 (br s, 2H), 2.60 (br s, 3H), 2.51-2.55 (m, 4H), 1.71 (br s, 2H), 1.57 (br s, 2H), 0.96 (br s, 6H).

### Example 37. methyl 1-(3-((3-((5-((2-(2,2-dimethylpyrrolidin-1-yl)ethyl) carbamoyl)-2-methylpyridin-3-yl)amino)-1-methyl-1H-pyrazolo[3,4-d]pyrimidin-6-yl)amino)phenyl)cyclopropanecarboxylate

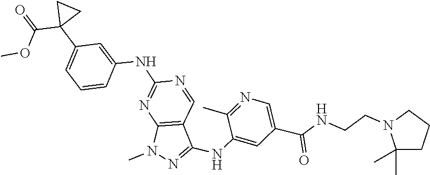 [SMILES: COC(=O)C1(c2cccc(Nc3ncc4c(Nc5cc(C(=O)NCCN6CCCC6(C)C)cnc5C)nn(C)c4n3)c2)CC1]

To a solution of methyl 1-(3-((3-chloro-1-methyl-1H-pyrazolo[3,4-d]pyrimidin-6-yl)amino)phenyl)-cyclopropanecarboxylate (85 mg, 0.24 mmol) dissolved in DMF (8 mL) was added 5-amino-N-(2-(2,2-dimethylpyrrolidin-1-yl)ethyl)-6-methylnicotinamide (105 mg, 0.35 mmol), Then Cs₂CO₃ (231 mg, 0.71 mmol), Brettphos (25 mg, 0.05 mmol) and Brettphos-Pd-G3 (43 mg, 0.05 mmol) was added. The mixture was charged with N₂ and then stirred at 120° C. for 12 hours before cooling to room temperature. The reaction was concentrated under reduced pressure to give crude product, which was purified by preparative high-performance liquid chromatography over Column: Xtimate C18 150*40 mm*5 um to give the title compound methyl 1-(3-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)phenyl)cyclopropanecarboxylate (60.2 mg, 20%) as a white solid. LCMS (ESI): mass calcd. for C₃₂H₃₉N₉O₃, 597.7; m/z found, 598.3 [M+H]⁺. ¹H NMR (400 MHz, DMSO-d₆) δ 9.87 (s, 1H), 9.03 (s, 1H), 8.65 (d, J=1.79 Hz, 1H), 8.41-8.50 (m, 3H), 7.92 (s, 1H), 7.76 (d, J=7.69 Hz, 1H), 7.26 (t, J=7.93 Hz, 1H), 6.97 (d, J=7.63 Hz, 1H), 3.78 (s, 3H), 3.55-3.59 (m, 3H), 3.21-3.32 (m, 4H), 2.71-2.79 (m, 2H), 2.58-2.62 (m, 3H), 1.67 (br d, J=6.91 Hz, 2H), 1.45-1.56 (m, 4H), 1.20-1.25 (m, 2H), 0.92 (s, 6H).

### Example 38. 1-(3-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)phenyl)cyclopropanecarboxylic acid

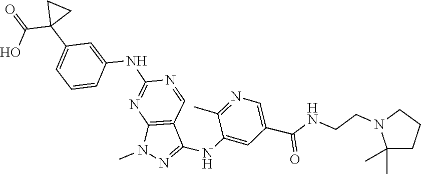 [SMILES: Cc1ncc(C(=O)NCCN2CCCC2(C)C)cc1Nc1nn(C)c2nc(Nc3cccc(C4(C(=O)O)CC4)c3)ncc12]

To a solution of methyl 1-(3-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)phenyl)cyclopropanecarboxylate (50 mg, 0.08 mmol) in tetrahydrofuran (3 mL) and water (1 mL) was added lithium hydroxide (54 mg, 1.3 mmol) and water (2 mL) at room temperature. The reaction mixture was stirred at 25° C. for 2 days. The mixture was adjusted to pH=4-5 with HCl (aq, 1 M). The reaction was concentrated under reduced pressure to give crude product, which was purified by preparative high-performance liquid chromatography over Column: Phenomenex Gemini-NX C18 75*30 mm*3 um to give the title compound 1-(3-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)phenyl)Cyclopropane-carboxylic acid (32.1 mg, 68%) as yellow solid. LCMS (ESI): mass calcd. for C₃₁H₃₇N₉O₃, 583.7; m/z found, 584.5 [M+H]⁺. ¹H NMR (400 MHz, DMSO-d₆) δ 9.84 (s, 1H), 9.03 (s, 1H), 8.66 (d, J=1.67 Hz, 1H), 8.40-8.51 (m, 3H), 8.19 (s, 1H), 7.92 (s, 1H), 7.74 (br d, J=8.11 Hz, 1H), 7.24 (t, J=7.87 Hz, 1H), 6.95 (d, J=7.51 Hz, 1H), 3.64-3.89 (m, 3H), 3.33 (br s, 2H), 2.81 (br t, J=7.15 Hz, 2H), 2.61 (s, 3H), 2.54-2.57 (m, 2H), 1.64-1.74 (m, 2H), 1.51-1.59 (m, 2H), 1.41-1.49 (m, 2H), 1.10-1.18 (m, 2H), 0.95 (s, 6H).

### Example 39. N-(2-(2,2-dimethylpyrrolidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

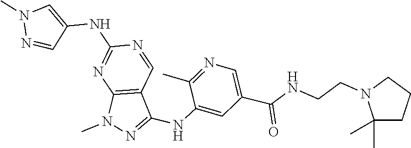 [SMILES: Cc1ncc(C(=O)NCCN2CCCC2(C)C)cc1Nc1nn(C)c2nc(Nc3cnn(C)c3)ncc12]

To a solution of 5-((6-chloro-1-methyl-1H-pyrazolo[3,4-d]pyrimidin-3-yl)amino)-N-(2-(2,2-dimethyl-pyrrolidin-1-yl)ethyl)-6-methylnicotinamide (100 mg, 0.23 mmol) dissolved in DMF (10 mL) was added 1-methyl-1H-pyrazol-4-amine (33 mg, 0.34 mmol), Then Cs₂CO₃ (221 mg, 0.68 mmol), Brettphos (24 mg, 0.05 mmol) and Tris(dibenzylideneacetone)dipalladium(0) (41 mg, 0.05 mmol) was added. The mixture was charged with N₂ and then stirred at 80° C. for 12 hours before cooling to room temperature. The reaction was concentrated under reduced pressure to give crude product, which was purified by preparative high-performance liquid chromatography over Column: Boston Prime C18 150*30 mm*5 um to give the title compound N-(2-(2,2-dimethylpyrrolidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (54.7 mg, 23%) as a light brown solid. LCMS (ESI): mass calcd. for C₂₅H₃₃N₁₁O, 503.6; m/z found, 504.4 [M+H]⁺. ¹H NMR (400 MHz, METHANOL-d₄) δ 8.88 (s, 1H), 8.76 (d, J=1.76 Hz, 1H), 8.48 (d, J=2.01 Hz, 1H), 8.12 (s, 1H), 7.67 (s, 1H), 3.93 (s, 3H), 3.88 (s, 3H), 3.52 (t, J=7.03 Hz, 2H), 2.92 (br t, J=6.90 Hz, 2H), 2.66-2.72 (m, 5H), 1.83 (br d, J=6.78 Hz, 2H), 1.67-1.72 (m, 2H), 1.05 (s, 6H).

### Example 40. N-(2-(2,2-dimethylpyrrolidin-1-yl)ethyl)-6-methyl-5-((1-methyl-6-((2-methyl-2H-1,2,3-triazol-4-yl)amino)-1H-pyrazolo[3,4-d]pyrimidin-3-yl)amino)nicotinamide

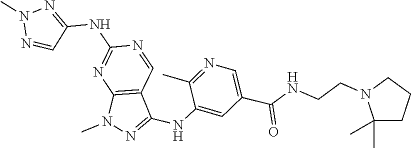 [SMILES: Cc1ncc(C(=O)NCCN2CCCC2(C)C)cc1Nc1nn(C)c2nc(Nc3cnn(C)n3)ncc12]

To a solution of 5-((6-chloro-1-methyl-1H-pyrazolo[3,4-d]pyrimidin-3-yl)amino)-N-(2-(2,2-dimethylpyrrolidin-1-yl)ethyl)-6-methylnicotinamide (100 mg, 0.23 mmol) dissolved in DMF (10 mL) was added 2-methyl-2H-1,2,3-triazol-4-amine hydrobromide (61 mg, 0.34 mmol), Then Cs₂CO₃ (221 mg, 0.68 mmol), Brettphos (24 mg, 0.05 mmol) and Tris(dibenzylideneacetone)dipalladium(0) (41 mg, 0.05 mmol) was added. The mixture was charged with N₂ and then stirred at 80° C. for 12 hours before cooling to room temperature. The reaction was concentrated under reduced pressure to give crude product, which was purified by preparative high-performance liquid chromatography over Column: Xtimate C18 150*40 mm*5 um to give the title compound N-(2-(2,2-dimethylpyrrolidin-1-yl)ethyl)-6-methyl-5-((1-methyl-6-((2-methyl-2H-1,2,3-triazol-4-yl)amino)-1H-pyrazolo[3,4-d]pyrimidin-3-yl)amino)nicotinamide (62.3 mg, 55%) as a light yellow solid. LCMS (ESI): mass calcd. for C₂₄H₃₂N₁₂O, 504.6; m/z found, 505.4 [M+H]⁺. 1H NMR (400 MHz, METHANOL-d₄) δ 8.96 (s, 1H), 8.77 (d, J=2.01 Hz, 1H), 8.48 (d, J=2.01 Hz, 1H), 8.21 (s, 1H), 4.91-5.01 (m, 1H), 4.12 (s, 3H), 3.89 (s, 3H), 3.52 (t, J=6.90 Hz, 2H), 2.91 (br t, J=7.28 Hz, 2H), 2.65-2.71 (m, 4H), 1.79-1.88 (m, 2H), 1.66-1.72 (m, 2H), 1.05 (s, 6H).

### Example 41. 5-((6-((2-aminopyridin-4-yl)amino)-1-methyl-1H-pyrazolo[3,4-d]pyrimidin-3-yl)amino)-N-(2-(2,2-dimethylpyrrolidin-1-yl)ethyl)-6-methylnicotinamide

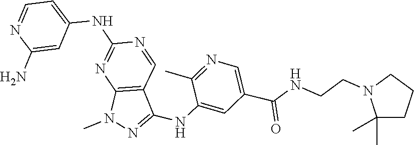 [SMILES: Cc1ncc(C(=O)NCCN2CCCC2(C)C)cc1Nc1nn(C)c2nc(Nc3ccnc(N)c3)ncc12]

To a solution of 5-((6-chloro-1-methyl-1H-pyrazolo[3,4-d]pyrimidin-3-yl)amino)-N-(2-(2,2-dimethylpyrrolidin-1-yl)ethyl)-6-methylnicotinamide (150 mg, 0.34 mmol) dissolved in DMF (10 mL) was added tert-butyl (4-aminopyridin-2-yl)carbamate (106 mg, 0.51 mmol), Then Cs₂CO₃ (331 mg, 1.0 mmol), Brettphos (36 mg, 0.07 mmol) and Tris(dibenzylideneacetone)dipalladium(0) (62 mg, 0.07 mmol) was added. The mixture was charged with N₂ and then stirred at 80° C. for 12 hours before cooling to room temperature. The reaction was concentrated under reduced pressure to give crude product, which was purified by preparative high-performance liquid chromatography over Column: Xtimate C18 150*40 mm*5 um to give the title compound 5-((6-((2-aminopyridin-4-yl)amino)-1-methyl-1H-pyrazolo[3,4-d]pyrimidin-3-yl)amino)-N-(2-(2,2-dimethylpyrrolidin-1-yl)ethyl)-6-methylnicotinamide (17.5 mg, 10%) as a light yellow solid. LCMS (ESI): mass calcd. for C₂₆H₃₃N₁₁O, 515.6; m/z found, 516.3 [M+H]⁺. ¹H NMR (400 MHz, METHANOL-d₄) δ 9.00 (s, 1H), 8.80 (d, J=1.76 Hz, 1H), 8.49 (d, J=1.76 Hz, 1H), 7.76 (d, J=5.77 Hz, 1H), 7.50 (d, J=1.76 Hz, 1H), 6.93 (dd, J=6.02, 2.01 Hz, 1H), 3.94 (s, 3H), 3.49-3.55 (m, 2H), 2.93 (br t, J=7.28 Hz, 2H), 2.68-2.72 (m, 5H), 1.83-1.89 (m, 2H), 1.68-1.73 (m, 2H), 1.06 (s, 6H).

### Example 42. N-(2-(2,2-dimethylpyrrolidin-1-yl)ethyl)-6-methyl-5-((1-methyl-6-(pyridin-3-ylamino)-1H-pyrazolo[3,4-d]pyrimidin-3-yl)amino)nicotinamide

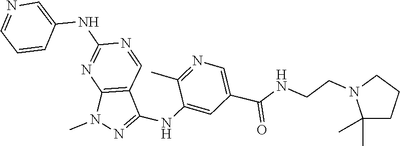 [SMILES: Cc1ncc(C(=O)NCCN2CCCC2(C)C)cc1Nc1nn(C)c2nc(Nc3cccnc3)ncc12]

### Step a: ethyl 6-methyl-5-((1-methyl-6-(pyridin-3-ylamino)-1H-pyrazolo[3,4-d]pyrimidin-3-yl)amino)nicotinate

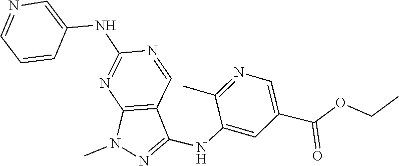 [SMILES: CCOC(=O)c1cnc(C)c(Nc2nn(C)c3nc(Nc4cccnc4)ncc23)c1]

To a solution of ethyl 5-((6-chloro-1-methyl-1H-pyrazolo[3,4-d]pyrimidin-3-yl)amino)-6-methylnicotinate (200 mg, 0.55 mmol) dissolved in DMF (10 mL) was added 3-Aminopyridine (61.8 mg 0.66 mmol), Then Cs₂CO₃ (534 mg 1.6 mmol), Brettphos (58.7 mg 0.11 mmol) and Brettphos-Pd-G3 (99.2 mg 0.11 mmol) was added. The mixture was charged with N₂. The mixture was stirred at 120° C. for 12 hours. The mixture was cooled to room temperature and filtered. The filtrate was concentrated to afford crude compound. The crude compound was purified by preparative high-performance liquid chromatography over Column: Welch Xtimate C18 100*40 mm*3 um to give the title compound ethyl 6-methyl-5-((1-methyl-6-(pyridin-3-ylamino)-1H-pyrazolo[3,4-d]pyrimidin-3-yl)amino)nicotinate (86 mg, 34%) as a yellow powder. LCMS (ESI): mass calcd. for C₂₀H₂₀N₈O₂, 404.4; m/z found, 405.2 [M+H]⁺.

### Step b: N-(2-(2,2-dimethylpyrrolidin-1-yl)ethyl)-6-methyl-5-((1-methyl-6-(pyridin-3-ylamino)-1H-pyrazolo[3,4-d]pyrimidin-3-yl)amino)nicotinamide

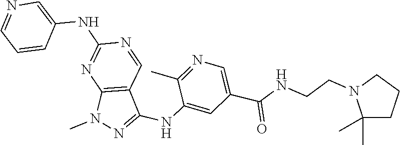 [SMILES: Cc1ncc(C(=O)NCCN2CCCC2(C)C)cc1Nc1nn(C)c2nc(Nc3cccnc3)ncc12]

To a solution of 2-(2,2-dimethylpyrrolidin-1-yl)ethanamine (46.2 mg, 0.33 mmol) in DCE (0.5 mL) was added Trimethylaluminium (2.0 mol/1 in toluene, 0.16 mL, 0.33 mmol) slowly at 0° C. under N₂. The mixture was charged with N₂. The mixture was stirred at 0° C. for 30 min. Then a solution of ethyl 6-methyl-5-((1-methyl-6-(pyridin-3-ylamino)-1H-pyrazolo [3,4-d] pyrimidin-3-yl) amino) nicotinate (76 mg 0.16 mmol) in DCE (0.5 mL) was added slowly. The mixture was stirred at 60° C. for 12 hours. The mixture was cooled to 0° C. Water (1 mL) was added slowly and the mixture was filtered. The filtrate was concentrated to afford to give crude compound. The crude compound was purified by preparative high-performance liquid chromatography over Column: Welch Xtimate C18 100*40 mm*3 um to give the title compound N-(2-(2,2-dimethylpyrrolidin-1-yl)ethyl)-6-methyl-5-((1-methyl-6-(pyridin-3-ylamino)-1H-pyrazolo[3,4-d]pyrimidin-3-yl)amino)nicotinamide (20.3 mg, 23%) as a red powder. LCMS (ESI): mass calcd. for C₂₆H₃₂N₁₀O, 500.5 m/z found, 501.3 [M+H]⁺. ¹H NMR (400 MHz, DMSO-d₆) δ 10.08 (s, 1H), 9.07 (s, 1H), 9.01 (br s, 1H), 8.66 (s, 2H), 8.38 (br d, J=8.58 Hz, 1H), 8.19-8.24 (m, 2H), 7.37 (dd, J=8.23, 4.53 Hz, 1H), 3.80 (s, 3H), 3.41 (br d, J=5.60 Hz, 2H), 2.97 (br t, J=7.03 Hz, 2H), 2.70 (br t, J=6.38 Hz, 2H), 2.61 (s, 3H), 2.54-2.55 (m, 1H), 1.75 (br d, J=6.68 Hz, 2H), 1.63-1.69 (m, 2H) 1.03 (s, 6H).

### Example 43. methyl 2-(3-((3-((5-(3-(2,2-dimethylpyrrolidin-1-yl)propan-amido)-2-methylpyridin-3-yl)amino)-1-methyl-1H-pyrazolo[3,4-d]pyrimidin-6-yl)amino) phenyl)acetate

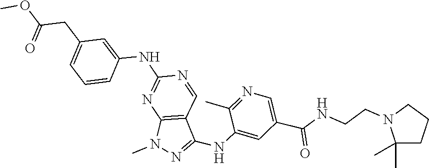 [SMILES: COC(=O)Cc1cccc(Nc2ncc3c(Nc4cc(C(=O)NCCN5CCCC5(C)C)cnc4C)nn(C)c3n2)c1]

### Step a: 5-amino-6-methylnicotinic acid

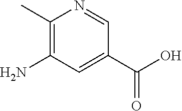

To a solution of ethyl 5-amino-6-methylnicotinate (5 g, 27.74 mmol) in methanol (15 mL) and THF (15 mL) and water (15 mL) was added sodium hydroxide (1.119 g, 27.74 mmol) at room temperature. The reaction mixture was stirred at 50° C. for 15 minutes before cooling to room-temperature. The mixture was adjusted to pH=3-4 with HCl (aq, 2 M). The mixture was filtered and washed with water (10 mL×3). The solid was evaporated under vacuum to give the desired product 5-amino-6-methylnicotinic acid (3.1 g, 93%) as yellow solid. ¹H NMR (400 MHz, DMSO-d₆) δ 8.17 (d, J=1.54 Hz, 1H), 7.67 (s, 1H), 2.39 (s, 3H).

### Step b: 5-amino-N-(2-(2,2-dimethylpyrrolidin-1-yl)ethyl)-6-methylnicotinamide

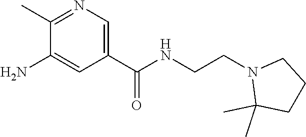 [SMILES: Cc1ncc(C(=O)NCCN2CCCC2(C)C)cc1N]

To a solution of 5-amino-6-methylnicotinic acid (3 g, 19.72 mmol), 2-(3H-[1,2,3]triazolo[4,5-b]pyridin-3-yl)-1,1,3,3-tetramethylisouronium hexafluorophosphate(V) (14.99 mg, 39.44 mmol), and N-ethyl-N-isopropylpropan-2-amine (13.04 mL, 78.8 mmol) in N,N-dimethylformamide (30 mL) was added 2-(2,2-dimethylpyrrolidin-1-yl)acetonitrile (2.8 g, 19.72 mmol). The mixture was stirred at 50° C. for 16 hours. The reaction mixture was concentrated under vacuum to give the crude product, which was purified by column chromatography over silica gel (eluent: MeOH:ethyl acetate=1:1) to give the title compound 5-amino-N-(2-(2,2-dimethylpyrrolidin-1-yl)ethyl)-6-methylnicotinamide (11 g, 66%) as a yellow solid. LCMS (ESI): mass calcd. for C₁₅H₂₄N₄O, 276.37; m/z found, 277.3 [M+H]⁺.

### Step c: ethyl 4-hydrazinyl-2-(methylthio)pyrimidine-5-carboxylate

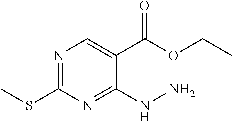

To a solution of ethyl 4-chloro-2-(methylthio)pyrimidine-5-carboxylate (25 g, 107.44 mmol) and EtOH (200 mL) was added hydrazine hydrate (16.1 g, 322.32 mmol). The mixture was stirred at 25° C. for 2 hours. The reaction mixture was filtered and the filter cake was concentrated under vacuum to give the crude product ethyl 4-hydrazinyl-2-(methylthio)pyrimidine-5-carboxylate (22 g, 90%) as a white solid. LCMS (ESI): mass calcd. for C₈H₁₂N₄O₂S, 228.27; m/z found, 229.1 [M+H]⁺.

### Step d: 6-(methylthio)-1H-pyrazolo[3,4-d]pyrimidin-3-ol

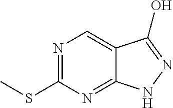

To a solution of ethyl 4-hydrazinyl-2-(methylthio)pyrimidine-5-carboxylate (20 g, 39.44 mmol) in KOH solution (250 mL, 4M). The mixture was stirred at 25° C. for 15 minutes. The reaction mixture was cooled to 0° C. and was acidified with 25 percent aq. AcOH. The resulting precipitate was filtered off, dried under reduced pressure and co-evaporated with toluene to afford the title compound 6-(methylthio)-1H-pyrazolo[3,4-d]pyrimidin-3-ol (18 g, 97%) as a yellow solid. ¹H NMR (400 MHz, DMSO-d₆): δ 8.63 (s, 1H), 2.47 (s, 3H), 1.91 (s, 1H).

### Step e: 3-chloro-6-(methylthio)-1H-pyrazolo[3,4-d]pyrimidine

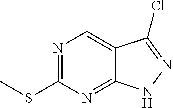

To a solution of 6-(methylthio)-1H-pyrazolo[3,4-d]pyrimidin-3-ol (8 g, 43.9 mmol) in POCl₃ (80 mL). The mixture was stirred at 90° C. for overnight. The reaction was quenched with sat.NaHCO₃ (20 mL) and the mixture was extracted with DCM (20 mL*3). The organic layer was dried with Na₂SO₄, filtered and concentrated under vacuum to give the crude product 3-chloro-6-(methylthio)-1H-pyrazolo[3,4-d]pyrimidine (10 g, 74%) as a white solid. LCMS (ESI): mass calcd. for C₆H₅ClN₄S, 200.65; m/z found, 200.8 [M+H]⁺. ¹H NMR (DMSO-d₆, 400 MHz): δ 14.14 (br s, 1H), 9.07 (s, 1H), 2.53 (s, 3H).

### Step f: 3-chloro-6-(methylthio)-1H-pyrazolo[3,4-d]pyrimidine

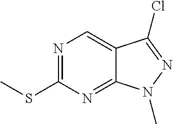

To a solution of 3-chloro-6-(methylthio)-1H-pyrazolo[3,4-d]pyrimidine (7 g, 26.1 mmol), and sodium hydride (1.5 g, 39.14 mmol) in N,N-dimethylformamide (100 mL) was added iodomethane (15.5 g, 109.614 mmol). The mixture was stirred at 0° C. for 15 minutes. The resulting mixture was quenched with water (50 mL) and extracted with ethyl acetate (100 mL×3). The organic was collected, dried over anhydrous Na₂SO₄, filtered and evaporated under vacuum to give the crude product 3-chloro-6-(methylthio)-1H-pyrazolo[3,4-d]pyrimidine (1.2 g, 21%) as colorless oil. LCMS (ESI): mass calcd. for C₇H₇ClN₄S, 214.67; m/z found, 215.1 [M+H]⁺.

### Step g: 3-chloro-1-methyl-6-(methylsulfonyl)-1H-pyrazolo[3,4-d]pyrimidine

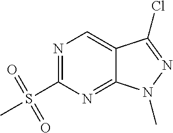

To a solution of 3-chloro-1-methyl-6-(methylthio)-1H-pyrazolo[3,4-d]pyrimidine (500 mg, 2.33 mmol), sodium periodate (992 mg, 4.66 mmol), and ruthenium(III) chloride (19.192 mg, 0.1 mmol) in water (7 mL) was added THF (7 mL). The mixture was stirred at 25° C. for 1 hours. The resulting mixture was quenched with water (50 mL) and extracted with ethyl acetate (100 mL×3). The organic was collected, dried over anhydrous Na₂SO₄, filtered and evaporated under vacuum to give the crude product 3-chloro-6-(methylthio)-1H-pyrazolo[3,4-d]pyrimidine (550 mg, 98%) as yellow solid.

### Step h: 2-(3-((3-chloro-1-methyl-1H-pyrazolo[3,4-d]pyrimidin-6-yl)amino)phenyl)acetic acid

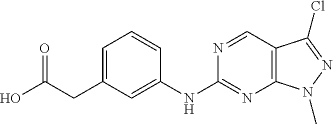 [SMILES: Cn1nc(Cl)c2cnc(Nc3cccc(CC(=O)O)c3)nc21]

To a solution of 3-chloro-1-methyl-6-(methylsulfonyl)-1H-pyrazolo[3,4-d]pyrimidine (340 mg, 1.38 mmol) and 2-(3-aminophenyl)acetic acid (417 mg, 2.76 mmol) in DMSO (10 mL). The mixture was stirred at 80° C. for 24 h. The reaction mixture was concentrated under vacuum to give the crude product, which was purified by preparative high-performance liquid chromatography over Column: Boston Prime C18 150*30 mm*5 um to give the title compound 2-(3-((3-chloro-1-methyl-1H-pyrazolo[3,4-d]pyrimidin-6-yl)amino)phenyl)acetic acid (230 mg, 94%) as a white solid. LCMS (ESI): mass calcd. for C₁₄H₁₂ClN₅O₂, 317.73; m/z found, 317.9 [M+H]⁺. ¹H NMR (400 MHz, DMSO-d₆) δ 12.31 (s, 1H), 10.11 (s, 1H), 8.94 (s, 1H), 7.87 (s, 1H), 7.67 (br d, J=9.26 Hz, 1H), 7.24 (t, J=7.83 Hz, 1H), 6.88 (d, J=7.50 Hz, 1H), 3.84 (s, 3H), 3.52 (s, 2H).

### Step i: methyl 2-(3-((3-chloro-1-methyl-1H-pyrazolo[3,4-d]pyrimidin-6-yl)amino)phenyl) acetate

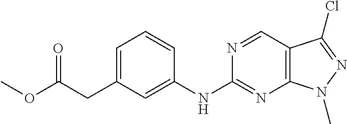 [SMILES: COC(=O)Cc1cccc(Nc2ncc3c(Cl)nn(C)c3n2)c1]

To a solution of 2-(3-((3-chloro-1-methyl-1H-pyrazolo[3,4-d]pyrimidin-6-yl)amino)phenyl)acetic acid (250 mg, 26.1 mmol) in HCl/MeOH (7.9 mL) was stirred at room temperature for overnight. The reaction mixture was concentrated under vacuum to give the crude product. The crude product was dissolved with water (10 mL) and adjusted pH to 7 with sat. NaHCO₃. The aqueous was extracted with DCM (50 mL*3) and the organic layers were collected, filtered and concentrated under vacuum to give the crude product methyl 2-(3-((3-chloro-1-methyl-1H-pyrazolo[3,4-d]pyrimidin-6-yl)amino)phenyl)acetate (250 mg, 99%) as a white solid. LCMS (ESI): mass calcd. for C₁₅H₁₄ClN₅O₂, 331.757; m/z found, 332.3 [M+H]⁺.

### Step j: methyl 2-(3-((3-((5-((2-(2,2-dimethylpyrrolidin-1-yl)ethyl)carbamoyl)-2-methylpyri-din-3-yl)amino)-1-methyl-1H-pyrazolo[3,4-d]pyrimidin-6-yl)amino)phenyl)acetate

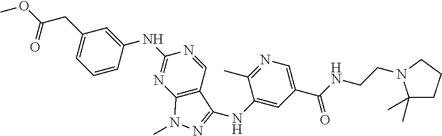 [SMILES: COC(=O)Cc1cccc(Nc2ncc3c(Nc4cc(C(=O)NCCN5CCCC5(C)C)cnc4C)nn(C)c3n2)c1]

To a solution of methyl 2-(3-((3-chloro-1-methyl-1H-pyrazolo[3,4-d]pyrimidin-6-yl)amino)phenyl)acetate (250 mg, 0.75 mmol), 5-amino-N-(2-(2,2-dimethylpyrrolidin-1-yl)ethyl)-6-methylnicotinamide (229 mg, 0.83 mmol), Cesium carbonate (736 mg, 2.26 mmol) and Brettphos (81 mg, 0.15 mmol) in N,N-dimethylformamide (5 mL) was added Brettphos-Pd-G3 (137 mg, 0.15 mmol). The mixture was stirred at 120° C. for 12 hours. The reaction mixture was concentrated under vacuum to give the crude product, which was purified by preparative high-performance liquid chromatography over Column: Boston Prime C18 150*30 mm*5 um to give the title compound 2-(3-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)phenyl)acetate (21 mg, 4%) as a white solid. LCMS (ESI): mass calcd. for C₃₀H₃₇N₉O₃, 571.673; m/z found, 572.1 [M+H]⁺. ¹H NMR (400 MHz, DMSO-d₆): δ 9.85 (s, 1H), 8.98 (s, 1H), 8.61 (d, J=1.5 Hz, 1H), 7.88 (s, 1H), 7.69 (br d, J=7.9 Hz, 1H), 7.23 (t, J=7.9 Hz, 1H), 6.84 (br d, J=7.3 Hz, 1H), 3.76 (s, 3H), 3.63 (s, 3H), 3.60 (s, 5H), 2.68-2.75 (m, 4H), 2.57 (s, 5H), 1.60-1.69 (m, 1H), 1.60-1.69 (m, 1H), 1.45-1.54 (m, 2H), 0.88 (s, 6H).

### Example 44. 2-(3-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)phenyl)-acetic acid

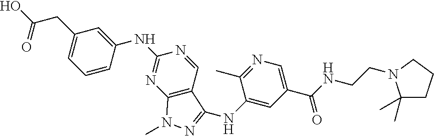 [SMILES: Cc1ncc(C(=O)NCCN2CCCC2(C)C)cc1Nc1nn(C)c2nc(Nc3cccc(CC(=O)O)c3)ncc12]

### Step a: 2-(3-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)phenyl)acetic acid

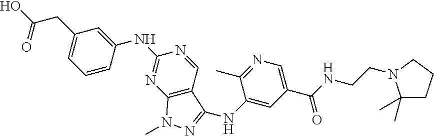 [SMILES: Cc1ncc(C(=O)NCCN2CCCC2(C)C)cc1Nc1nn(C)c2nc(Nc3cccc(CC(=O)O)c3)ncc12]

To a solution of methyl 2-(3-((3-chloro-1-methyl-1H-pyrazolo[3,4-d]pyrimidin-6-yl)amino)phenyl)acetate (250 mg, 0.75 mmol), 5-amino-N-(2-(2,2-dimethylpyrrolidin-1-yl)ethyl)-6-methylnicotinamide (229 mg, 0.83 mmol), cesium carbonate (737 mg, 2.26 mmol) and Brettphos (81 mg, 0.15 mmol) in N,N-dimethylformamide (5 mL) was added Brettphos-Pd-G3 (137 mg, 0.15 mmol). The mixture was stirred at 120° C. for 12 hours. The reaction mixture was concentrated under vacuum to give the crude product, which was purified by preparative high-performance liquid chromatography over Column: Boston Prime C18 150*30 mm*5 um to give the title compound 2-(3-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)phenyl)acetic acid (35 mg, 8%) as a white solid. LCMS (ESI): mass calcd. for C₂₉H₃₅N₉O₃, 557.647; m/z found, 558.1 [M+H]⁺. ¹H NMR (DMSO-d₆, 400 MHz): δ 12.6 (br s, 1H), 9.79-9.90 (m, 1H), 8.99 (s, 1H), 8.61 (br d, J=2.0 Hz, 1H), 8.39-8.49 (m, 2H), 8.21 (s, 1H), 7.88 (s, 1H), 7.69 (br d, J=8.2 Hz, 1H), 7.23 (br t, J=7.8 Hz, 1H), 6.84 (br d, J=7.3 Hz, 1H), 3.76 (s, 3H), 3.56-3.67 (m, 8H), 2.73-2.79 (m, 3H), 1.65 (br d, J=6.4 Hz, 2H), 1.40-1.57 (m, 2H), 0.87-0.95 (m, 6H).

### Example 45. N-(2-(2,2-dimethylpyrrolidin-1-yl)ethyl)-5-((6-((1,1-dioxido-tetrahydro-2H-thiopyran-4-yl)amino)-1-methyl-1H-pyrazolo[3,4-d]pyrimidin-3-yl)amino)-6-methylnicotinamide

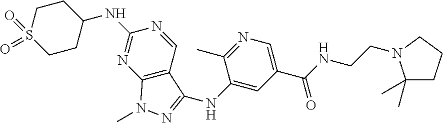 [SMILES: Cc1ncc(C(=O)NCCN2CCCC2(C)C)cc1Nc1nn(C)c2nc(NC3CCS(=O)(=O)CC3)ncc12]

### Step a: 4-((3-chloro-1-methyl-1H-pyrazolo[3,4-d]pyrimidin-6-yl)amino)tetrahydro-2H-thiopyran 1,1-dioxide

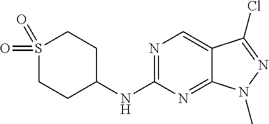 [SMILES: Cn1nc(Cl)c2cnc(NC3CCS(=O)(=O)CC3)nc21]

To a solution of 3-chloro-1-methyl-6-(methylsulfonyl)-1H-pyrazolo[3,4-d]pyrimidine (1.3 g, 5.27 mmol) and 4-aminotetrahydro-2H-thiopyran 1,1-dioxide (978 mg, 5.27 mmol) in DMSO (20 mL) was stirred at 120° C. for overnight. The resulting mixture was quenched with water (30 mL) and extracted with ethyl acetate (50 mL×3). The combined organic phases were dried over anhydrous Na₂SO₄ and filtered. The filtrate was concentrated under reduced pressure to give crude product, which was purified by preparative high-performance liquid chromatography over Column: Phenomenex Gemini-NX 80*40 mm*3 um to give the title compound 4-((3-chloro-1-methyl-1H-pyrazolo[3,4-d]pyrimidin-6-yl)amino)tetrahydro-2H-thiopyran 1,1-dioxide (100 mg, 98%) as a white solid. LCMS (ESI): mass calcd. for C₁₁H₁₄ClN₅O₂S, 315.7; m/z found, 315.8, [M+H]⁺.

### Step b: N-(2-(2,2-dimethylpyrrolidin-1-yl)ethyl)-5-((6-((1,1-dioxidotetrahydro-2H-thiopyran-4-yl)amino)-1-methyl-1H-pyrazolo[3,4-d]pyrimidin-3-yl)amino)-6-methylnicotinamide

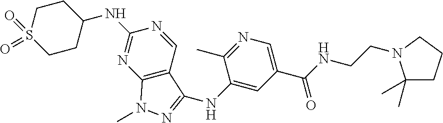 [SMILES: Cc1ncc(C(=O)NCCN2CCCC2(C)C)cc1Nc1nn(C)c2nc(NC3CCS(=O)(=O)CC3)ncc12]

To a solution of 4-((3-chloro-1-methyl-1H-pyrazolo[3,4-d]pyrimidin-6-yl)amino)tetrahydro-2H-thiopyran 1,1-dioxide (25 mg, 0.08 mmol), 5-amino-N-(2-(2,2-dimethylpyrrolidin-1-yl)ethyl)-6-methylnicotinamide (24 mg, 0.09 mmol), cesium carbonate (78 mg, 0.23 mmol) and Brettphos (9 mg, 0.02 mmol) in N,N-dimethylformamide (0.5 mL) was added Brettphos-Pd-G3 (14 mg, 0.02 mmol). The mixture was stirred at 120° C. for 12 hours. The reaction mixture was concentrated under vacuum to give the crude product, which was purified by preparative high-performance liquid chromatography over Column: Boston Prime C18 150*30 mm*5 um to give the title compound N-(2-(2,2-dimethylpyrrolidin-1-yl)ethyl)-5-((6-((1,1-dioxidotetrahydro-2H-thiopyran-4-yl)amino)-1-methyl-1H-pyrazolo[3,4-d]pyrimidin-3-yl)amino)-6-methylnicotinamide (10 mg, 20%) as a white solid. LCMS (ESI): mass calcd. for C₂₆H₃₇N₉O₃S, 555.695; m/z found, 556.3 [M+H]⁺. ¹H NMR (METHANOL-d₄, 400 MHz): δ 8.81 (s, 1H), 8.77 (d, J=2.0 Hz, 1H), 8.50 (br s, 1H), 8.48 (d, J=1.8 Hz, 1H), 4.61 (br s, 1H), 4.24-4.33 (m, 1H), 3.77 (s, 3H), 3.70 (br t, J=6.1 Hz, 2H), 3.47 (br s, 1H), 3.27 (br s, 1H), 3.19 (br s, 4H), 2.65 (s, 3H), 2.41 (br d, J=10.1 Hz, 2H), 2.18-2.31 (m, 2H), 2.02-2.13 (m, 2H), 1.92-2.01 (m, 2H), 1.32 (s, 6H).

### Example 46. methyl 2-(3-((3-((5-((2-(2,2-dimethylpyrrolidin-1-yl)ethyl) carbamoyl)pyridin-3-yl)amino)-1-methyl-1H-pyrazolo[3,4-d]pyrimidin-6-yl)amino) phenyl)-2-methylpropanoate

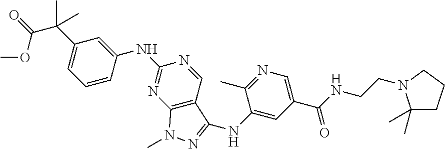 [SMILES: COC(=O)C(C)(C)c1cccc(Nc2ncc3c(Nc4cc(C(=O)NCCN5CCCC5(C)C)cnc4C)nn(C)c3n2)c1]

### Step a: methyl 2-(3-((3-chloro-1-methyl-1H-pyrazolo[3,4-d]pyrimidin-6-yl)amino)phenyl)-2-methylpropanoate

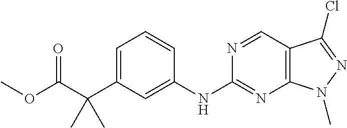 [SMILES: COC(=O)C(C)(C)c1cccc(Nc2ncc3c(Cl)nn(C)c3n2)c1]

To a solution of 3-chloro-1-methyl-6-(methylsulfonyl)-1H-pyrazolo[3,4-d]pyrimidine (493 mg, 2.0 mmol) in formamide (20 mL) was added methyl 2-(3-aminophenyl)-2-methylpropanoate (400 mg, 2.0 mmol) at room temperature. The resulting mixture was stirred at 100° C. for overnight before cooling to room temperature and filtered. The filtrate was concentrated under reduced pressure to give crude product, which was purified by preparative high-performance liquid chromatography over Column: Phenomenex Gemini NX C18 150*40 mm*5 um to give the title compound methyl 2-(3-((3-chloro-1-methyl-1H-pyrazolo[3,4-d]pyrimidin-6-yl)amino)phenyl)-2-methylpropanoate (277 mg, 39%) as a white solid. LCMS (ESI): mass calcd. for C₁₇H₁₈ClN₅O₂, 359.8; m/z found, 360.1 [M+H]⁺.

### Step b: methyl 2-(3-((3-((5-((2-(2,2-dimethylpyrrolidin-1-yl)ethyl)carbamoyl)pyridin-3-yl)amino)-1-methyl-1H-pyrazolo[3,4-d]pyrimidin-6-yl)amino)phenyl)-2-methylpropanoate

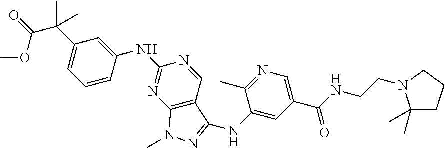 [SMILES: COC(=O)C(C)(C)c1cccc(Nc2ncc3c(Nc4cc(C(=O)NCCN5CCCC5(C)C)cnc4C)nn(C)c3n2)c1]

To a solution of methyl 2-(3-((3-chloro-1-methyl-1H-pyrazolo[3,4-d]pyrimidin-6-yl)amino)phenyl)-2-methylpropanoate (277 mg, 0.77 mmol), 5-amino-N-(2-(2,2-dimethylpyrrolidin-1-yl)ethyl)-6-methylnicotinamide (243 mg, 0.85 mmol), Brettphos-Pd-G3 (174 mg, 0.19 mmol), cesium bicarbonate (752 mg, 2.3 mmol), Brettphos (103 mg, 0.19 mmol) in DMF (12 mL). The resulting mixture was stirred at 120° C. for overnight before cooling to room-temperature. The resulting mixture was quenched with water (30 mL) and extracted with ethyl acetate (50 mL*3). The combined organic layers were washed with sat. NaCl (20 mL*3) and then concentrated under vacuum to give the crude product, which was purified by preparative high-performance liquid chromatography over Column: Xtimate C18 150*40 mm*5 um to give the title compound methyl 2-(3-((3-((5-((2-(2,2-dimethylpyrrolidin-1-yl)ethyl)carbamoyl)pyridin-3-yl)amino)-1-methyl-1H-pyrazolo[3,4-d]pyrimidin-6-yl)amino)phenyl)-2-methylpropanoate (185 mg, 34%) as a yellow solid. LCMS(ESI): mass calcd. for C₃₂H₄₁N₉O₃, 599.7, m/z found, 600.4 [M+H]⁺. ¹H NMR (400 MHz, METHANOL-d₄) δ 8.94 (s, 1H), 8.84 (d, J=1.9 Hz, 1H), 8.52 (d, J=1.8 Hz, 1H), 8.33 (br s, 2H), 8.14 (s, 1H), 7.58 (dd, J=1.6, 7.9 Hz, 1H), 7.31 (t, J=8.0 Hz, 1H), 7.04 (d, J=8.0 Hz, 1H), 3.90 (s, 3H), 3.78 (br t, J=6.2 Hz, 2H), 3.69 (s, 3H), 3.64 (br s, 2H), 2.70 (s, 3H), 2.21-2.11 (m, 2H), 2.10-2.04 (m, 2H), 1.63 (s, 6H), 1.42 (s, 6H).

### Example 47. 2-(3-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)phenyl)-2-methylpropanoic acid

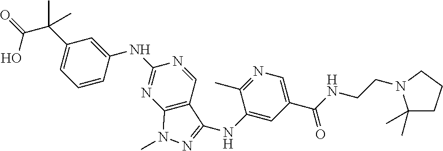 [SMILES: Cc1ncc(C(=O)NCCN2CCCC2(C)C)cc1Nc1nn(C)c2nc(Nc3cccc(C(C)(C)C(=O)O)c3)ncc12]

To a solution of methyl 2-(3-((3-((5-((2-(2,2-dimethylpyrrolidin-1-yl)ethyl)carbamoyl)-2-methylpyridin-3-yl)amino)-1-methyl-1H-pyrazolo[3,4-d]pyrimidin-6-yl)amino)phenyl)-2-methylpropanoate (165 mg, 0.27 mmol) and lithium hydroxide (32 mg, 1.35 mmol) in methanol: tetrahydrofuran: water (1.5 mL, 1:3:1) at 25° C. for 12 h. The reaction mixture was slowly acidified with a in aqueous solution of HCl (1M) (until pH=4). The reaction mixture was filtered and the filter cake, dried in vacuum to give the crude product, which was purified by preparative high-performance liquid chromatography over Column: Xtimate C18 150*40 mm*5 um to give the title compound 2-(3-((3-((5-((2-(2,2-dimethylpyrrolidin-1-yl)ethyl)carbamoyl)-2-methylpyridin-3-yl) amino)-1-methyl-1H-pyrazolo[3,4-d]pyrimidin-6-yl)amino)phenyl)-2-methylpropanoic acid (85 mg, 53%) as a yellow solid. LCMS (ESI): mass calcd. for C₃₁H₃₉N₉O₃, 585.7; m/z found, 586.3 [M+H]⁺. ¹H NMR (400 MHz, DMSO-d₆) δ 9.90 (s, 1H), 9.04 (s, 1H), 8.67 (d, J=1.8 Hz, 1H), 8.54-8.42 (m, 3H), 8.29 (br s, 2H), 7.55 (br d, J=8.5 Hz, 1H), 7.26 (t, J=7.9 Hz, 1H), 6.98 (br d, J=7.4 Hz, 1H), 3.81 (s, 3H), 3.35-3.29 (m, 2H), 2.83-2.75 (m, 1H), 2.79 (br t, J=7.2 Hz, 1H), 2.61 (s, 3H), 2.54 (br d, J=5.5 Hz, 2H), 1.74-1.62 (m, 2H), 1.59-1.53 (m, 2H), 1.51 (s, 6H), 0.94 (s, 6H).

### Example 48. N-(2-(2,2-dimethylpyrrolidin-1-yl)ethyl)-4-methyl-5-((1-methyl-6-(pyrimidin-5-ylamino)-1H-pyrazolo[3,4-d]pyrimidin-3-yl)amino)thiophene-2-carboxamide

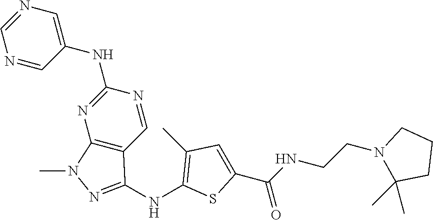 [SMILES: Cc1cc(C(=O)NCCN2CCCC2(C)C)sc1Nc1nn(C)c2nc(Nc3cncnc3)ncc12]

### Step a: methyl 5-(2,4-dichloropyrimidine-5-carboxamido)-4-methylthiophene-2-carboxylate

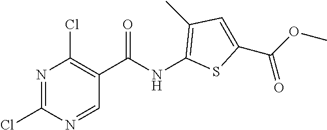 [SMILES: COC(=O)c1cc(C)c(NC(=O)c2cnc(Cl)nc2Cl)s1]

To a solution of methyl 5-amino-4-methylthiophene-2-carboxylate (1.4 g, 8.0 mmol) in dioxane (80 mL) was added a solution of 2,4-dichloropyrimidine-5-carbonyl chloride (2 g, 9.5 mmol) in Dioxane (20 mL) dropwise, maintained at 70° C. Then the reaction mixture was stirred at 85° C. for 15 h. The mixture was cooled down room temperature then concentrated under vacuum to give crude. The crude was purified by column chromatography over silica gel (eluent: petroleum ether:ethyl acetate=3:1) to afford methyl 5-(2,4-dichloropyrimidine-5-carboxamido)-4-methylthiophene-2-carboxylate (2.8 g, 85%) as an orange solid. ¹H NMR (400 MHz, CDCl₃) δ 9.30 (s, 1H), 9.16 (s, 1H), 7.57 (s, 1H), 3.90 (s, 3H), 2.31 (s, 3H).

### Step b: methyl 5-(2-chloro-4-(1-methylhydrazinyl)pyrimidine-5-carboxamido)-4-methylthiophene-2-carboxylate

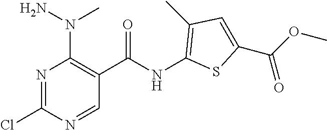 [SMILES: COC(=O)c1cc(C)c(NC(=O)c2cnc(Cl)nc2N(C)N)s1]

To a solution of methyl 5-(2,4-dichloropyrimidine-5-carboxamido)-4-methylthiophene-2-carboxylate (1.7 g, 4.9 mmol) in THF (30 mL) was added methylhydrazine (566 mg, 4.9 mmol) and Et₃N (1 g, 10.3 mmol) at 0° C. The resulting mixture was stirred at room temperature for 1 h and then concentrated under vacuum to give the crude product. The crude was triturated with H₂O (100 mL) then filtered. The filter cake was dried under vacuum to afford methyl 5-(2-chloro-4-(1-methylhydrazinyl)pyrimidine-5-carboxamido)-4-methylthiophene-2-carboxylate (1.2 g, 68%) as a yellow solid. LCMS (ESI): mass calcd. for C₁₃H₁₄ClN₅O₃S, 355.1; m/z found, 356.1 [M+H]⁺.

### Step c: methyl 5-((6-chloro-1-methyl-1H-pyrazolo[3,4-d]pyrimidin-3-yl)amino)-4-methylthiophene-2-carboxylate

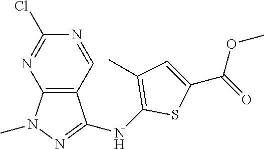

To a solution of methyl 5-(2-chloro-4-(1-methylhydrazinyl)pyrimidine-5-carboxamido)-4-methylthiophene-2-carboxylate (5.4 g, 14 mmol) in toluene (135 mL) was added PCl₅ (2.9 g, 14 mmol) at room temperature. The reaction mixture was stirred at 120° C. for 12 h before cooling to room-temperature. The mixture was evaporated under vacuum to give crude. The crude was triturated with MeOH (20 mL). The mixture was filtered. The filter cake was washed with H₂O (10 mL) and MeOH (10 mL). The filter cake was dried under vacuum to afford product methyl 5-((6-chloro-1-methyl-1H-pyrazolo[3,4-d]pyrimidin-3-yl)amino)-4-methylthiophene-2-carboxylate (2 g, 34%) as a yellow solid. LCMS (ESI): mass calcd. for C₁₃H₁₂ClN₈O₂S, 337.0; m/z found, 338.1 [M+H]⁺.

### Step d: methyl 4-methyl-5-((1-methyl-6-(pyrimidin-5-ylamino)-1H-pyrazolo[3,4-d]pyrimidin-3-yl)amino)thiophene-2-carboxylate

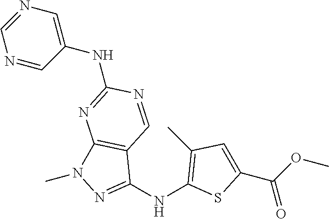 [SMILES: COC(=O)c1cc(C)c(Nc2nn(C)c3nc(Nc4cncnc4)ncc23)s1]

To a mixture of methyl 5-((6-chloro-1-methyl-1H-pyrazolo[3,4-d]pyrimidin-3-yl)amino)-4-methylthiophene-2-carboxylate (200 mg, 0.59 mmol), pyrimidin-5-amine (84 mg, 0.89 mmol), Brettphos-Pd-G3 (134 mg, 0.15 mmol) and Brettphos (79 mg, 0.15 mmol) in DMF (5 mL) was added Cs₂CO₃ (579 mg, 1.8 mmol) at room temperature. The reaction mixture was purged with N₂ for 2 minutes. Then the reaction mixture was stirred at 90° C. for 16 h. The reaction mixture was cooled down room temperature. H₂O (50 mL) was added. Then the mixture was filtered. The filter cake was washed with H₂O (20 mL). The filter cake was dried under vacuum to give crude. The crude was triturated with TBME (20 mL). The mixture was filtered. The filter cake was dried under vacuum to give crude of title compound methyl 4-methyl-5-((1-methyl-6-(pyrimidin-5-ylamino)-1H-pyrazolo[3,4-d]pyrimidin-3-yl)amino)thiophene-2-carboxylate (180 mg, 62%) as a brown solid. LCMS (ESI): mass calcd. for C₁₇H₁₆N₈O₂S, 396.1; m/z found, 397.1 [M+H]⁺.

### Step e: N-(2-(2,2-dimethylpyrrolidin-1-yl)ethyl)-4-methyl-5-((1-methyl-6-(pyrimidin-5-ylamino)-1H-pyrazolo[3,4-d]pyrimidin-3-yl)amino)thiophene-2-carboxamide

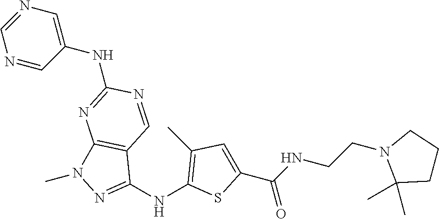 [SMILES: Cc1cc(C(=O)NCCN2CCCC2(C)C)sc1Nc1nn(C)c2nc(Nc3cncnc3)ncc12]

To a mixture of methyl 4-methyl-5-((1-methyl-6-(pyrimidin-5-ylamino)-1H-pyrazolo[3,4-d]pyrimidin-3-yl)amino)thiophene-2-carboxylate (120 mg, 0.24 mmol) and 2-(2,2-dimethylpyrrolidin-1-yl)ethanamine (142 mg, 1.0 mmol) in THF (8 mL) was added Me₃Al (0.6 mL, 1.2 mmol, 2M in toluene) under N₂ at 0° C. The resulting mixture was stirred at 60° C. for 15 h before cooled down room temperature. The reaction mixture was quenched with MeOH (10 mL) and filtered over celite. The filtrate was concentrated to the crude product. The crude was purified by preparative high-performance liquid chromatography over Column: Phenomenex Gemini-NX C18 75*30 mm*3 um to give the title compound N-(2-(2,2-dimethylpyrrolidin-1-yl)ethyl)-4-methyl-5-((1-methyl-6-(pyrimidin-5-ylamino)-1H-pyrazolo[3,4-d]pyrimidin-3-yl)amino)thiophene-2-carboxamide (50 mg, 34%) as a yellow solid. LCMS (ESI): mass calcd. for C₂₄H₃₀N₁₀OS, 506.2; m/z found, 507.3 [M+H]⁺. ¹H NMR (400 MHz, DMSO-d₆) δ 10.23 (s, 1H), 9.61 (s, 1H), 9.29 (s, 2H), 9.15 (s, 1H), 8.82 (s, 1H), 8.18 (s, 1H), 7.41 (s, 1H), 3.82 (s, 3H), 2.80 (br t, J=7.2 Hz, 2H), 2.55 (br s, 4H), 2.24 (s, 3H), 1.76-1.66 (m, 2H), 1.61-1.53 (m, 2H), 0.96 (s, 6H).

### Example 49. N-(2-(4-methoxypiperidin-1-yl)ethyl)-4-methyl-5-((1-methyl-6-((1-methyl-1H-pyrazol-4-yl)amino)-1H-pyrazolo[3,4-d]pyrimidin-3-yl)amino)thiophene-2-carboxamide

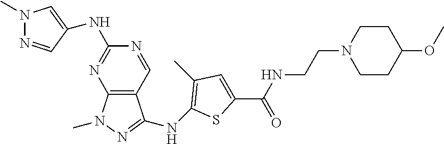

### Step a: methyl 4-methyl-5-((1-methyl-6-((1-methyl-1H-pyrazol-4-yl)amino)-1H-pyrazolo[3,4-d]pyrimidin-3-yl)amino)thiophene-2-carboxylate

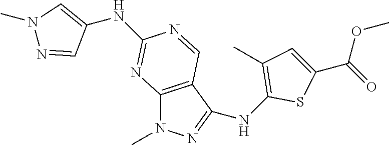

To a mixture of methyl 5-((6-chloro-1-methyl-1H-pyrazolo[3,4-d]pyrimidin-3-yl)amino)-4-methylthiophene-2-carboxylate (2 g, 4.7 mmol), 1-methyl-1H-pyrazol-4-amine (688 mg, 7.1 mmol), Brettphos-Pd-G3 (1070 mg, 1.2 mmol) and Brettphos (634 mg, 1.2 mmol) in DMF (60 mL) was added Cs₂CO₃ (4.6 g, 14 mmol) at room temperature. The reaction mixture was purged with N₂ for 10 minutes. Then the reaction mixture was stirred at 90° C. for 16 h. The reaction mixture was cooled down room temperature. H₂O (300 mL) was added. Then the mixture was filtered. The filter cake was washed with H₂O (100 mL). The filter cake was dried under vacuum to give crude. The crude was triturated with TBME (50 mL). The mixture was filtered. The filter cake was dried under vacuum to give crude of title compound methyl 4-methyl-5-((1-methyl-6-((1-methyl-1H-pyrazol-4-yl)amino)-1H-pyrazolo[3,4-d]pyrimidin-3-yl)amino)thiophene-2-carboxylate (1900 mg, 85%) as a brown solid. LCMS (ESI): mass calcd. for C₁₇H₁₈N₈O₂S, 398.1; m/z found, 399.2 [M+H]⁺.

### Step b: N-(2-(4-methoxypiperidin-1-yl)ethyl)-4-methyl-5-((1-methyl-6-((1-methyl-1H-pyrazol-4-yl)amino)-1H-pyrazolo[3,4-d]pyrimidin-3-yl)amino)thiophene-2-carboxamide

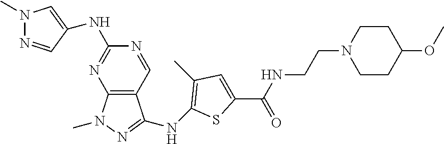

To a mixture of methyl 4-methyl-5-((1-methyl-6-((1-methyl-1H-pyrazol-4-yl)amino)-1H-pyrazolo[3,4-d]pyrimidin-3-yl)amino)thiophene-2-carboxylate (150 mg, 0.32 mmol) and 2-(4-methoxypiperidin-1-yl)ethanamine (184 mg, 0.8 mmol) in THF (9 mL) was added Me₃Al (0.48 mL, 0.96 mmol, 2M in toluene) under N₂ at 0° C. The resulting mixture was stirred at 60° C. for 15 h before cooled down room temperature. The reaction mixture was quenched with MeOH (10 mL) and filtered over celite. The filtrate was concentrated to the crude product. The crude was purified by preparative high-performance liquid chromatography over Column: Welch Xtimate C18 150*25 mm*5 um to give the title compound N-(2-(4-methoxypiperidin-1-yl)ethyl)-4-methyl-5-((1-methyl-6-((1-methyl-1H-pyrazol-4-yl)amino)-1H-pyrazolo[3,4-d]pyrimidin-3-yl)amino)thiophene-2-carboxamide (23 mg, 18%) as a yellow solid. LCMS (ESI): mass calcd. for C₂₄H₃₂N₁₀O₂S, 524.2; m/z found, 525.3 [M+H]⁺. ¹H NMR (400 MHz, METHANOL-d₆) δ 8.90 (s, 1H), 8.09 (s, 1H), 7.67 (s, 1H), 7.42 (s, 1H), 3.93 (s, 3H), 3.87 (s, 3H), 3.68 (br t, J=5.8 Hz, 2H), 3.55 (br s, 1H), 3.39 (s, 3H), 3.37 (s, 1H), 3.31-3.27 (m, 2H), 3.23-3.10 (m, 3H), 2.29 (s, 3H), 2.04 (br d, J=8.9 Hz, 2H), 1.95 (br s, 2H).

### Example 50. N-(2-(2,2-dimethylpyrrolidin-1-yl)ethyl)-4-fluoro-3-((1-methyl-6-(pyridazin-4-ylamino)-1H-pyrazolo[3,4-d]pyrimidin-3-yl)amino)benzamide

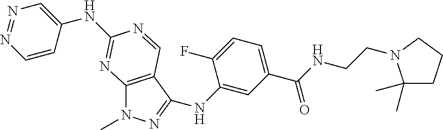 [SMILES: Cn1nc(Nc2cc(C(=O)NCCN3CCCC3(C)C)ccc2F)c2cnc(Nc3ccnnc3)nc21]

### Step a: ethyl 3-(2,4-dichloropyrimidine-5-carboxamido)-4-fluorobenzoate

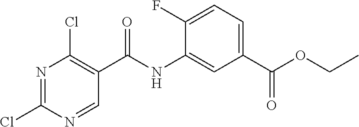 [SMILES: CCOC(=O)c1ccc(F)c(NC(=O)c2cnc(Cl)nc2Cl)c1]

To a solution of ethyl 3-amino-4-fluorobenzoate (953 mg, 5.2 mmol) in DCM (50 mL) was 2,4-dichloropyrimidine-5-carbonyl chloride (1 g, 4.7 mmol). The mixture was charged with N₂. The mixture was stirred at 25° C. overnight. The mixture was concentrated under vacuum to give a crude as yellow gum, which was purified by flash column chromatography over 40 g silica gel (eluent: dichloromethane:methanol from 100:0 to 90:10) to give the title compound ethyl 3-(2,4-dichloropyrimidine-5-carboxamido)-4-fluorobenzoate (985 mg, 54%) as yellow solid. LCMS (ESI): mass calcd. for C₁₄H₁₀Cl₂FN₃O₃, 358.2; m/z found, 358.1[M+H]⁺. ¹H NMR (400 MHz, DMSO-d₆) δ 10.86 (s, 1H), 9.13 (d, J=9.8 Hz, 1H), 8.70 (dd, J=2.1, 7.5 Hz, 1H), 7.87 (ddd, J=2.2, 4.8, 8.6 Hz, 1H), 7.51 (dd, J=8.8, 10.4 Hz, 1H), 4.35 (q, J=7.0 Hz, 2H), 1.34 (t, J=7.1 Hz, 3H).

### Step b: ethyl 3-(2-chloro-4-(1-methylhydrazinyl)pyrimidine-5-carboxamido)-4-fluorobenzoate

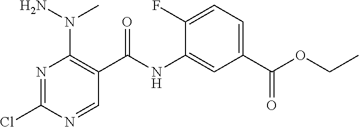 [SMILES: CCOC(=O)c1ccc(F)c(NC(=O)c2cnc(Cl)nc2N(C)N)c1]

To a solution of ethyl 3-(2,4-dichloropyrimidine-5-carboxamido)-4-fluorobenzoate (930 mg, 2.4 mmol) dissolved in THF (16 mL) was added TEA (604 mg, 48.8 mmol) and methyl hydrazine (40% in water, 820 mg, 7.1 mmol). The mixture was stirred at room temperature for 1 hour. The reaction mixture was concentrated under vacuum to give the crude product. The residue was diluted with ethyl acetate (20 mL). The solution was washed with H₂O (20 mL*2), dried over MgSO₄, filtered and concentrated under vacuum to afford the title compound ethyl 3-(2-chloro-4-(1-methylhydrazinyl)pyrimidine-5-carboxamido)-4-fluorobenzoate (622 mg, 49%) as yellow powder. LCMS (ESI): mass calcd. for C₁₅H₁₅ClFN₅O₃, 367.8; m/z found, 367.9[M+H]⁺.

### Step c: ethyl 3-((6-chloro-1-methyl-1H-pyrazolo[3,4-d]pyrimidin-3-yl)amino)-4-fluorobenzoate

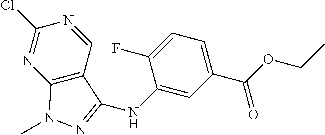 [SMILES: CCOC(=O)c1ccc(F)c(Nc2nn(C)c3nc(Cl)ncc23)c1]

To a solution of ethyl 3-(2-chloro-4-(1-methylhydrazinyl)pyrimidine-5-carboxamido)-4-fluorobenzoate (622 mg, 0.17 mmol) dissolved in toluene (16 mL) was added Phosphorus pentachloride (242 mg 1.2 mmol). The mixture was stirred at 120° C. for 16 hours. The residue was diluted with the solvent (dichloromethane:methanol=8/1 (500 mL)). The reaction mixture was washed with saturated aqueous NaHCO₃ (400 mL) and the organic layer was dried over MgSO₄, the mixture was concentrated under vacuum to give crude compound. The crude compound was purified by column chromatography over silica gel (eluent: dichloromethane:methanol=100:0 to 90:10) to give the title compound ethyl 3-((6-chloro-1-methyl-1H-pyrazolo[3,4-d]pyrimidin-3-yl)amino)-4-fluorobenzoate (476 mg, 89%) as yellow powder. LCMS (ESI): mass calcd. for C₁₅H₁₃ClFN₅O₂, 349.7; m/z found, 350.1 [M+H]⁺. ¹H NMR (400 MHz, DMSO-d₆) δ 9.64 (s, 1H), 9.37 (s, 1H), 9.02 (dd, J=2.0, 8.3 Hz, 111), 7.54 (ddd, J=2.1, 4.6, 8.4 Hz, 111), 7.36 (dd, J=8.5, 11.4 Hz, 111), 4.31-4.22 (m, 2H), 3.89-3.75 (m, 3H), 1.31-1.26 (m, 3H).

### Step d: ethyl 4-fluoro-3-((1-methyl-6-(pyridazin-4-ylamino)-1H-pyrazolo[3,4-d]pyrimidin-3-yl)amino)benzoate

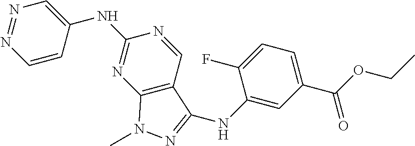 [SMILES: CCOC(=O)c1ccc(F)c(Nc2nn(C)c3nc(Nc4ccnnc4)ncc23)c1]

To a solution of ethyl 3-((6-chloro-1-methyl-1H-pyrazolo[3,4-d]pyrimidin-3-yl)amino)-4-fluorobenzoate (200 mg, 0.55 mmol) dissolved in DMF (10 mL) was added pyridazin-4-amine (82 mg 0.66 mmol), Then Cs₂CO₃ (534 mg 1.6 mmol), Brettphos (59 mg 0.11 mmol) and Brettphos-Pd-G3 (100 mg 0.11 mmol) was added. The mixture was charged with N₂. The mixture was stirred at 100° C. for 12 hours. The mixture was cooled to room temperature and filtered. The filtrate was concentrated to afford crude compound. Ethyl acetate (5 mL) was added to the mixture and the mixture was stirred at room temperature for 30 min. The mixture was filtered and the filter cake was dried in vacuum to give the title compound ethyl 4-fluoro-3-((1-methyl-6-(pyridazin-4-ylamino)-1H-pyrazolo[3,4-d]pyrimidin-3-yl)amino)benzoate (312 mg, 61%) as brown powder. LCMS (ESI): mass calcd. for C₁₉H₁₇FN₈O₂, 408.4; m/z found, 409.1 [M+H]⁺.

### Step e: 4-fluoro-3-((1-methyl-6-(pyridazin-4-ylamino)-1H-pyrazolo[3,4-d]pyrimidin-3-yl)amino)benzoic acid

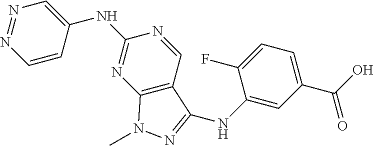 [SMILES: Cn1nc(Nc2cc(C(=O)O)ccc2F)c2cnc(Nc3ccnnc3)nc21]

To a solution of 2-(2,2-dimethylpyrrolidin-1-yl)ethanamine (300 mg, 0.37 mmol) in THE (3 mL), methanol (1 mL) and H₂O (1 mL) was added lithium hydroxide (78.6 mg, 1.9 mmol) at room temperature. The reaction mixture was stirred at 20° C. for 2 h. The mixture was adjusted to pH=3˜4 with HCl (aq, 2 M). The mixture was evaporated under vacuum to give the crude compound 4-fluoro-3-((1-methyl-6-(pyridazin-4-ylamino)-1H-pyrazolo[3,4-d]pyrimidin-3-yl)amino)benzoic acid as yellow oil. The crude compound was used for next step without purification. LCMS (ESI): mass calcd. for C₁₇H₁₃FN₈O₂, 380.3; m/z found, 381.0 [M+H]⁺.

### Step f: N-(2-(2,2-dimethylpyrrolidin-1-yl)ethyl)-4-fluoro-3-((1-methyl-6-(pyridazin-4-ylamino)-1H-pyrazolo[3,4-d]pyrimidin-3-yl)amino)benzamide

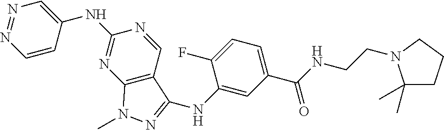 [SMILES: Cn1nc(Nc2cc(C(=O)NCCN3CCCC3(C)C)ccc2F)c2cnc(Nc3ccnnc3)nc21]

To a solution of 4-fluoro-3-((1-methyl-6-(pyridazin-4-ylamino)-1H-pyrazolo[3,4-d]pyrimidin-3-yl)amino)benzoic acid (120 mg, 0.17 mmol), HATU (97 mg, 0.25 mmol) and N,N-diisopropylethylamine (87 mg, 0.68 mmol) in DMF (3 mL) was added 2-(2,2-dimethylpyrrolidin-1-yl)ethanamine (31 mg, 0.22 mmol). The resulting mixture was stirred at room temperature for 16 h and then concentrated under vacuum to give the crude product, which was purified by preparative high-performance liquid chromatography over Column: Phenomenex Gemini NX C18 150*40 mm*5 um to give the crude compound. The crude compound was purified by supercritical fluid chromatography over Column: DAICEL CHIRALPAK AS 250*30 mm*10 um to give title compound: N-(2-(2,2-dimethylpyrrolidin-1-yl)ethyl)-4-fluoro-3-((1-methyl-6-(pyridazin-4-ylamino)-1H-pyrazolo[3,4-d]pyrimidin-3-yl)amino)benzamide (12.7 mg, 14%) as a white solid. LCMS (ESI): mass calcd. for C₂₅H₂₉FN₁₀O, 504.6; m/z found, 505.2 [M+H]⁺. ¹H NMR (400 MHz, METHANOL-d4) δ 9.32 (d, J=2.26 Hz, 1H), 9.01 (s, 1H), 8.81 (d, J=6.08 Hz, 1H), 8.69 (dd, J=8.17, 2.21 Hz, 1H), 8.38 (dd, J=6.14, 2.80 Hz, 1H), 7.28 (ddd, J=8.43, 4.44, 2.27 Hz, 1H), 7.11 (dd, J=11.09, 8.46 Hz, 1H), 3.84 (s, 3H), 3.42 (t, J=6.91 Hz, 2H), 2.84-2.92 (m, 2H), 2.61 (t, J=6.97 Hz, 2H), 1.67-1.78 (m, 2H), 1.58-1.66 (m, 2H), 0.97 (s, 6H).

### Example 51. N-(2-(2,2-dimethylpyrrolidin-1-yl)ethyl)-4-fluoro-3-((1-methyl-6-(pyrimidin-5-ylamino)-1H-pyrazolo[3,4-d]pyrimidin-3-yl)amino)benzamide

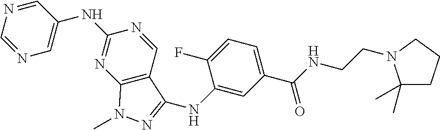 [SMILES: Cn1nc(Nc2cc(C(=O)NCCN3CCCC3(C)C)ccc2F)c2cnc(Nc3cncnc3)nc21]

### Step a: ethyl 4-fluoro-3-((1-methyl-6-(pyrimidin-5-ylamino)-1H-pyrazolo[3,4-d]pyrimidin-3-yl)amino)benzoate

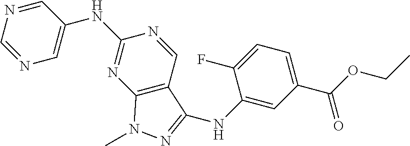 [SMILES: CCOC(=O)c1ccc(F)c(Nc2nn(C)c3nc(Nc4cncnc4)ncc23)c1]

To a solution of ethyl ethyl 3-((6-chloro-1-methyl-1H-pyrazolo[3,4-d]pyrimidin-3-yl)amino)-4-fluorobenzoate (150 mg, 0.33 mmol), 5-aminopyrimidine (47 mg, 0.49 mmol), cesium carbonate (321 mg, 0.98 mmol) and Brettphos (35 mg, 0.06 mmol) in DMF (10 mL) was added Brettphos-Pd-G3 (59 mg, 0.06 mmol) under N₂. The resulting mixture was stirred at 80° C. under N₂ for 16 h before cooled to 25° C. The reaction mixture was diluted with ethyl acetate (50 mL) and filtered over celite. The filtrate was concentrated to the crude product, which was treated with water (7 mL). The residue was filtered and the filter cake was washed with water (10 mL), dried in vacuum to give the crude product, which was triturated with methyl tert-butyl ether/ethyl acetate/methanol=25:5:1 (20 mL) and filtered to afford ethyl 4-fluoro-3-((1-methyl-6-(pyrimidin-5-ylamino)-1H-pyrazolo[3,4-d]pyrimidin-3-yl)amino)benzoate (251 mg, 90%) as a brown solid. LCMS (ESI): mass calcd. for C₁₉H₁₇FN₈O₂, 408.4; m/z found, 409.0[M+H]⁺.

### Step b: 4-fluoro-3-((1-methyl-6-(pyrimidin-5-ylamino)-1H-pyrazolo[3,4-d]pyrimidin-3-yl)amino)benzoic acid

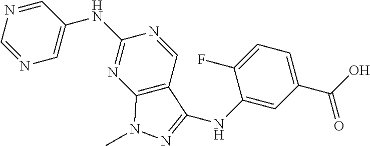 [SMILES: Cn1nc(Nc2cc(C(=O)O)ccc2F)c2cnc(Nc3cncnc3)nc21]

To a solution of ethyl 4-fluoro-3-((1-methyl-6-(pyrimidin-5-ylamino)-1H-pyrazolo[3,4-d] pyrimidin-3-yl)amino)benzoate (241 mg, 0.28 mmol) in methanol: tetrahydrofuran: water (2 mL, 1:3:1) was added lithium hydroxide (34 mg, 1.42 mmol), the reaction mixture was stirred at 25° C. for 2 h. The reaction mixture was adjusted to pH=4 slowly with aqueous solution of HCl (1M). The reaction mixture was filtered and the filter cake, dried in vacuum to give the title compound 4-fluoro-3-((1-methyl-6-(pyrimidin-5-ylamino)-1H-pyrazolo[3,4-d]pyrimidin-3-yl)amino)benzoic acid (107 mg, 98%) as a brown solid. LCMS (ESI): mass calcd. for C₁₇H₁₃FN₈O₂, 380.3; m/z found, 381.1 [M+H]⁺.

### Step c: N-(2-(2,2-dimethylpyrrolidin-1-yl)ethyl)-4-fluoro-3-((1-methyl-6-(pyrimidin-5-yl-amino)-1H-pyrazolo[3,4-d]pyrimidin-3-yl)amino)benzamide

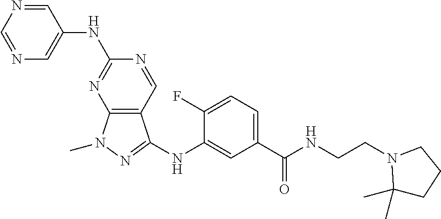 [SMILES: Cn1nc(Nc2cc(C(=O)NCCN3CCCC3(C)C)ccc2F)c2cnc(Nc3cncnc3)nc21]

To a solution of 4-fluoro-3-((1-methyl-6-(pyrimidin-5-ylamino)-1H-pyrazolo[3,4-d]pyrimidin-3-yl)amino)benzoic acid (107 mg, 0.28 mmol), HATU (214 mg, 0.56 mmol) and N,N-diisopropylethylamine (145 ul, 1.12 mmol) in N,N-dimethylformamide (9 mL) was added 2-(2,2-dimethylpyrrolidin-1-yl)ethanamine (44 mg, 0.31 mmol). The mixture stirred at 25° C. for 16 hours and then concentrated under vacuum to give the crude product, which was purified by preparative high-performance liquid chromatography over column: Phenomenex Gemini NX C18 150*40 mm*5 um to give the title compound N-(2-(2,2-dimethylpyrrolidin-1-yl)ethyl)-4-fluoro-3-((1-methyl-6-(pyrimidin-5-ylamino)-1H-pyrazolo[3,4-d]pyrimidin-3-yl)amino)benzamide (35 mg, 23%) as a yellow solid. LCMS (ESI): mass calcd. for C₂₅H₂₉FN₁₀O, 504.5; m/z found, 505.4 [M+H]⁺. ¹H NMR (400 MHz, DMSO-d₆) δ 10.24 (s, 1H), 9.30 (s, 2H), 9.25 (s, 2H), 8.84-8.74 (m, 2H), 8.59 (br s, 1H), 7.47 (br s, 1H), 7.34 (br dd, J=8.5, 11.1 Hz, 1H), 3.84 (s, 3H), 3.61-3.43 (m, 4H), 2.81 (br d, J=15.6 Hz, 2H), 1.87-1.67 (m, 4H), 1.10 (br s, 6H).

### Example 52. N-(2-(2,2-dimethylpyrrolidin-1-yl)ethyl)-4-fluoro-3-((1-methyl-6-(pyrimidin-4-ylamino)-1H-pyrazolo[3,4-d]pyrimidin-3-yl)amino)benzamide

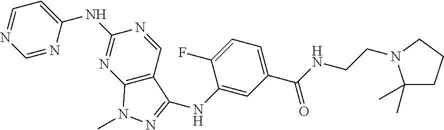 [SMILES: Cn1nc(Nc2cc(C(=O)NCCN3CCCC3(C)C)ccc2F)c2cnc(Nc3ccncn3)nc21]

### Step a: 4-fluoro-3-((1-methyl-6-(pyrimidin-4-ylamino)-1H-pyrazolo[3,4-d]pyrimidin-3-yl)amino)benzoate

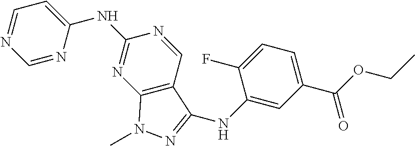 [SMILES: CCOC(=O)c1ccc(F)c(Nc2nn(C)c3nc(Nc4ccncn4)ncc23)c1]

To a solution of ethyl 3-((6-chloro-1-methyl-1H-pyrazolo[3,4-d]pyrimidin-3-yl)amino)-4-fluorobenzoate (300 mg, 0.66 mmol), pyrimidin-4-amine (93 mg, 0.99 mmol), cesium carbonate (642 mg, 1.95 mmol) and Brettphos (71 mg, 0.13 mmol) in DMF (7 mL) was added Brettphos-Pd-G3 (119 mg, 0.13 mmol) under N₂. The resulting mixture was stirred at 90° C. under N₂ for 12 h before cooled to 25° C. The reaction mixture was diluted with ethyl acetate (50 mL) and filtered over celite. The filtrate was concentrated to the crude product, which was treated with water (30 mL). The residue was filtered and the filter cake was washed with water (50 mL), dried in vacuum to give afford the crude product, which was triturated with methyl tert-butyl ether/ethyl acetate/methanol=25:5:1 (50 mL) and filtered to afford 4-fluoro-3-((1-methyl-6-(pyrimidin-4-ylamino)-1H-pyrazolo[3,4-d]pyrimidin-3-yl)amino)benzoate (350 mg, 98%) as a brown solid. LCMS (ESI): mass calcd. for C₁₉H₁₇FN₈O₂, 408.4; m/z found, 409.2 [M+H]⁺.

### Step b: 4-fluoro-3-((1-methyl-6-(pyrimidin-4-ylamino)-1H-pyrazolo[3,4-d]pyrimidin-3-yl)amino)benzoic acid

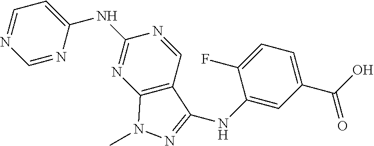 [SMILES: Cn1nc(Nc2cc(C(=O)O)ccc2F)c2cnc(Nc3ccncn3)nc21]

To a solution of ethyl 4-fluoro-3-((1-methyl-6-(pyrimidin-4-ylamino)-1H-pyrazolo[3,4-d]pyrimidin-3-yl)amino)benzoate (350 mg, 0.86 mmol) in methanol: tetrahydrofuran: water (3.75 mL, 1:3:1) was added lithium hydroxide (103 mg, 4.28 mmol), the mixture was stirred at 25° C. for 2 h. The reaction mixture was adjusted to pH=4 slowly with aqueous solution of HCl (1M). The reaction mixture was filtered and the filter cake, dried in vacuum to give the title compound 4-fluoro-3-((1-methyl-6-(pyrimidin-4-ylamino)-1H-pyrazolo[3,4-d]pyrimidin-3-yl)amino)benzoic acid (100 mg, 31%) as a brown solid.

### Step c: N-(2-(2,2-dimethylpyrrolidin-1-yl)ethyl)-4-fluoro-3-((1-methyl-6-(pyrazin-2-ylamino)-1H-pyrazolo[3,4-d]pyrimidin-3-yl)amino)benzamide

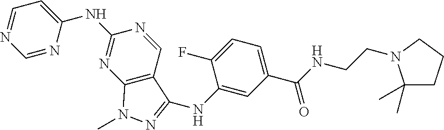 [SMILES: Cn1nc(Nc2cc(C(=O)NCCN3CCCC3(C)C)ccc2F)c2cnc(Nc3ccncn3)nc21]

To a solution of 4-fluoro-3-((1-methyl-6-(pyrimidin-4-ylamino)-1H-pyrazolo[3,4-d]pyrimidin-3-yl)amino)benzoic acid (90 mg, 0.24 mmol), HATU (180 mg, 0.47 mmol) and N,N-diisopropylethylamine (156 ul, 0.95 mmol) in N,N-dimethylformamide (9 mL) was added 2-(2,2-dimethylpyrrolidin-1-yl)ethanamine (37 mg, 0.26 mmol). The mixture was stirred at 25° C. for 2 hours and then concentrated under vacuum to give the crude product, which was purified by preparative high-performance liquid chromatography over Column: Phenomenex Gemini NX C18 150*40 mm*5 um to give the title compound N-(2-(2,2-dimethylpyrrolidin-1-yl)ethyl)-4-fluoro-3-((1-methyl-6-(pyrimidin-4-ylamino)-1H-pyrazolo[3,4-d]pyrimidin-3-yl)amino)benzamide (28.4 mg, 24%) as a yellow solid. LCMS (ESI): mass calcd. for C₂₅H₂₉N₁₀O, 504.5; m/z found, 505.1 [M+H]⁺. ¹H NMR (400 MHz, METHANOL-d₄) δ 9.17 (s, 1H), 8.84 (dd, J=2.1, 8.2 Hz, 1H), 8.78 (s, 1H), 8.68 (dd, J=1.3, 6.1 Hz, 1H), 8.59 (d, J=6.1 Hz, 1H), 7.43 (ddd, J=2.2, 4.4, 8.5 Hz, 1H), 7.26 (dd, J=8.5, 11.1 Hz, 1H), 3.98 (s, 3H), 3.61 (br s, 2H), 3.15 (br d, J=1.7 Hz, 2H), 2.92 (br s, 2H), 1.97 (br s, 2H), 1.84 (br s, 2H), 1.19 (br s, 6H).

### Example 53. N-(2-(4-azaspiro[2.4]heptan-4-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

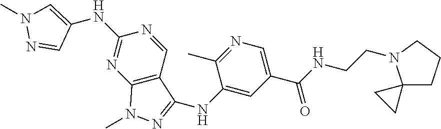 [SMILES: Cc1ncc(C(=O)NCCN2CCCC23CC3)cc1Nc1nn(C)c2nc(Nc3cnn(C)c3)ncc12]

### Step a: 2-(4-azaspiro[2.4]heptan-4-yl)acetonitrile

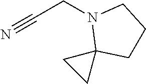

To a solution of 4-azaspiro[2.4]heptane; hydrochloride (800 mg, 6.0 mmol) and potassium carbonate (2.07 g, 14.9 mmol) in acetonitrile (10 mL) was added 2-bromoacetonitrile (0.77 g, 6.6 mmol) at room temperature. The resulting mixture was stirred at 50° C. for 12 h before cooling to room-temperature. The reaction mixture was filtered. The residue was washed with ethyl acetate (10 mL×3) and concentrated under reduced pressure to give crude product, which was purified by column chromatography over silica gel (eluent: petroleum ether:ethyl acetate=3:1) to give the title compound 2-(4-azaspiro[2.4]heptan-4-yl)acetonitrile (800 mg, 98%) as a yellow oil. LCMS (ESI): mass calcd. for C₈H₁₂N₂, 136.1; m/z found, 137.1 [M+H]⁺. ¹H NMR (400 MHz, CDCl₃) δ 3.33 (s, 2H), 3.03 (t, J=6.8 Hz, 2H), 2.00-1.92 (m, 2H), 1.87-1.79 (m, 2H), 0.79-0.70 (m, 2H), 0.57-0.50 (m, 2H).

### Step b: 2-(4-azaspiro[2.4]heptan-4-yl)ethanamine

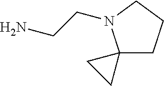

To a solution of 2-(4-azaspiro[2.4]heptan-4-yl)acetonitrile (800 mg, 5.87 mmol) in THF (20 mL) was added lithium aluminium hydride (446 mg, 11.7 mmol) by portions at 0° C. (ice/water), and the resulting mixture was stirred at 25° C. for 90 min. After cooled to 0° C., the reaction mixture was quenched with water (446 mg) and filtered. The filtrate was then concentrated to dryness under reduced pressure to afford the crude product 2-(4-azaspiro[2.4]heptan-4-yl)ethanamine (600 mg, 73%) as a colorless oil. LCMS (ESI): mass calcd. for C₈H₁₆N₂, 140.2; m/z found, 141.2[M+H]⁺. ¹H NMR (400 MHz, CDCl₃) δ 2.47-2.32 (m, 4H), 1.88 (t, J=6.4 Hz, 2H), 1.56-1.46 (m, 2H), 1.45-1.37 (m, 2H), 1.0 (br s, 2H), 0.39-0.30 (m, 2H), 0.05-0.03 (m, 2H).

### Step c: N-(2-(4-azaspiro[2.4]heptan-4-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

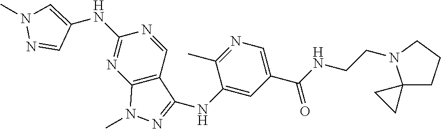 [SMILES: Cc1ncc(C(=O)NCCN2CCCC23CC3)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (130 mg, 0.34 mmol), HATU (195 mg, 0.5 mmol) and N,N-diisopropylethylamine (226.5 ul, 1.37 mmol) in N,N-dimethylformamide (5 mL) was added 2-(4-azaspiro[2.4]heptan-4-yl)ethanamine (62.5 mg, 0.44 mmol). The mixture stirred at 25° C. for 2 hours and then concentrated under vacuum to give the crude product, which was purified by preparative high-performance liquid column chromatography: Welch Xtimate C18 100*25 mm*3 um, then SFC column: DAICEL CHIRALPAK AS (250 mm*30 mm, 10 um) to give the title compound N-(2-(4-azaspiro[2.4]heptan-4-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (25.2 mg, 14%) as a white solid. LCMS (ESI): mass calcd. for C₂₅H₃₁N₁₁O, 501.6; m/z found, 502.4 [M+H]⁺. ¹H NMR (400 MHz, METHANOL-d₄) δ 8.85 (s, 1H), 8.74 (d, J=1.8 Hz, 1H), 8.44 (d, J=1.8 Hz, 1H), 8.09 (s, 1H), 7.65 (s, 1H), 3.90 (s, 3H), 3.85 (s, 3H), 3.48 (t, J=7.1 Hz, 2H), 2.93 (t, J=7.1 Hz, 2H), 2.64 (s, 3H), 2.50 (t, J=7.2 Hz, 2H), 2.00-1.90 (m, 2H), 1.85-1.79 (m, 2H), 0.81-0.76 (m, 2H), 0.48-0.42 (m, 2H).

### Example 54. N-(2-(4-oxa-7-azaspiro[2.5]octan-7-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

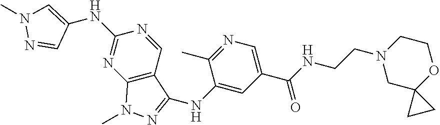 [SMILES: Cc1ncc(C(=O)NCCN2CCOC3(CC3)C2)cc1Nc1nn(C)c2nc(Nc3cnn(C)c3)ncc12]

### Step a: 2-(4-oxa-7-azaspiro[2.5]octan-7-yl)acetonitrile

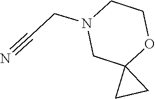 [SMILES: N#CCN1CCOC2(CC2)C1]

To a solution of 4-oxa-7-azaspiro [2.5]octane hydrochloride (500 mg, 3.34 mmol) and potassium carbonate (1.15 g, 8.3 mmol) in acetonitrile (8 mL) was added 2-bromoacetonitrile (228.5 ul, 3.67 mmol) at room-temperature. The resulting mixture was stirred at 50° C. for 12 h before cooling to room-temperature. The reaction mixture was filtered. The residue was washed with ethyl acetate (10 mL×3) and concentrated under reduced pressure to give crude product, which was purified by column chromatography over silica gel (eluent: petroleum ether:ethyl acetate=3:1) to give the title compound 2-(4-oxa-7-azaspiro[2.5]octan-7-yl)acetonitrile (380 mg, 70%) as a colorless oil. LCMS (ESI): mass calcd. for C₈H₁₂N₂O, 152.19; m/z found, 153.2 [M+H]⁺. ¹H NMR (400 MHz, CDCl₃) δ 3.84-3.77 (m, 2H), 3.53 (s, 2H), 2.74-2.65 (m, 2H), 2.62-2.50 (m, 2H), 0.88-0.80 (m, 2H), 0.61-0.52 (m, 2H).

### Step b: 2-(4-oxa-7-azaspiro[2.5]octan-7-yl)ethanamine

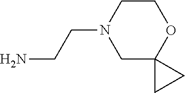 [SMILES: NCCN1CCOC2(CC2)C1]

To a solution of 2-(4-oxa-7-azaspiro[2.5]octan-7-yl)acetonitrile (360 mg, 2.36 mmol) in THF (20 mL) was added lithium aluminium hydride (179.5 mg, 4.73 mmol) by portions at 0° C. (ice/water) and the resulting mixture was stirred at 25° C. for 90 minutes. After cooled to 0° C., the reaction mixture was quenched with water (180 mg) and filtered. The filtrate was then concentrated to dryness under reduced pressure to afford the crude product 2-(4-oxa-7-azaspiro[2.5]octan-7-yl)ethanamine (320 mg, 87%) as a colorless oil. LCMS (ESI): mass calcd. for C₈H₁₆N₂O, 156.2; m/z found, 157.1[M+H]⁺. ¹H NMR (400 MHz, CDCl₃) δ 3.77-3.72 (m, 2H), 2.77 (t, J=6.3 Hz, 2H), 2.54-2.48 (m, 2H), 2.44-2.36 (m, 4H), 1.35 (br s, 2H), 0.81-0.73 (m, 2H), 0.54-0.46 (m, 2H).

### Step c: N-(2-(4-oxa-7-azaspiro[2.5]octan-7-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

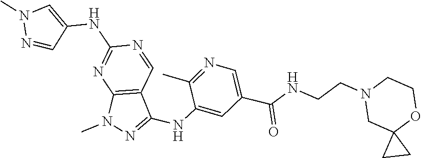 [SMILES: Cc1ncc(C(=O)NCCN2CCOC3(CC3)C2)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (150 mg, 0.39 mmol), HATU (225 mg, 0.59 mmol) and N,N-diisopropylethylamine (261 ul, 1.58 mmol) in N,N-dimethylformamide (5 mL) was added 2-(4-oxa-7-azaspiro[2.5]octan-7-yl)ethanamine (80 mg, 0.51 mmol). The mixture stirred at 25° C. for 2 hours and then concentrated under vacuum to give the crude product, which was purified by preparative high-performance liquid column chromatography: Welch Xtimate C18 150*30 mm*5 um to give the title compound N-(2-(4-oxa-7-azaspiro[2.5]octan-7-yl)ethyl)-6-methyl-5-((1-methyl-6-((1-methyl-1H-pyrazol-4-yl)amino)-1H-pyrazolo[3,4-d]pyrimidin-3-yl)amino)nicotin-amide (51.1 mg, 24%) as a gray solid. LCMS (ESI): mass calcd. for C₂₅H₃₁NO₂, 517.6; m/z found, 518.3 [M+H]⁺. ¹H NMR (400 MHz, METHANOL-d₄) δ 8.84 (s, 1H), 8.75 (d, J=1.8 Hz, 1H), 8.43 (d, J=1.8 Hz, 1H), 8.07 (s, 1H), 7.63 (s, 1H), 3.89 (s, 3H), 3.83 (s, 3H), 3.78-3.71 (m, 2H), 3.55 (t, J=6.6 Hz, 2H), 2.66-2.59 (m, 7H), 2.52 (s, 2H), 0.78-0.69 (m, 2H), 0.60-0.53 (m, 2H).

### Example 55. N-(2-(3,3-dimethylazetidin-1-yl)ethyl)-6-methyl-5-((1-methyl-6-(pyrimidin-5-ylamino)-1H-pyrazolo[3,4-d]pyrimidin-3-yl)amino)nicotinamide

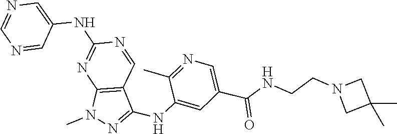 [SMILES: Cc1ncc(C(=O)NCCN2CC(C)(C)C2)cc1Nc1nn(C)c2nc(Nc3cncnc3)ncc12]

### Step a: 2-(3,3-dimethylazetidin-1-yl)acetonitrile

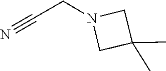

To a solution of 3,3-dimethylazetidine; hydrochloride (0.5 g, 4.1 mmol) and potassium carbonate (1.1 g, 8.2 mmol) in acetonitrile (10 mL) was added 2-bromoacetonitrile (542 mg, 4.5 mmol) at room temperature. The resulting mixture was stirred at 60° C. for 12 h before cooling to room-temperature. The resulting mixture was quenched with water (30 mL) and extracted with ethyl acetate (50 mL×3). The combined organic phases were dried over anhydrous Na₂SO₄ and filtered. The filtrate was concentrated under reduced pressure to give crude product, which was purified by column chromatography over silica gel (eluent: petroleum ether:ethyl acetate=3:1) to give the title compound 2-(3,3-dimethylazetidin-1-yl)acetonitrile (300 mg, 59%) as a pale yellow oil. ¹H NMR (400 MHz, METHANOL-d₄) δ 3.53 (s, 2H), 3.13 (s, 4H), 1.22 (s, 6H).

### Step b: 2-(3,3-dimethylazetidin-1-yl)ethanamine

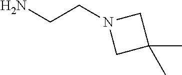

To a solution of 2-(3,3-dimethylazetidin-1-yl)acetonitrile (250 mg, 2.0 mmol) in THF (4 mL) was added lithium aluminium hydride (84 mg, 2.2 mmol) by portions at 0° C. (ice/water). The resultant mixture was stirred at 20° C. for 90 minutes before quenched with water (100 mg) at 0° C. The reaction mixture was filtered. And the filtrate was concentrated to dryness under reduced pressure to afford the crude product 2-(3,3-dimethylazetidin-1-yl)ethanamine (130 mg, 50%) as a colorless oil. ¹H NMR (400 MHz, METHANOL-d₄) δ 3.02 (s, 4H), 2.62-2.49 (m, 4H), 1.20 (s, 6H).

### Step c: N-(2-(3,3-dimethylazetidin-1-yl)ethyl)-6-methyl-5-((1-methyl-6-(pyrimidin-5-ylamino)-1H-pyrazolo[3,4-d]pyrimidin-3-yl)amino)nicotinamide

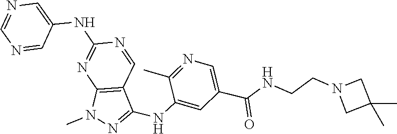 [SMILES: Cc1ncc(C(=O)NCCN2CC(C)(C)C2)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino) nicotinic acid (125 mg, 0.33 mmol), HATU (189 mg, 0.50 mmol) and N,N-diisopropylethylamine (128 mg, 1.0 mmol) in DMF (5 mL) was added 2-(3,3-dimethylazetidin-1-yl)ethanamine (80 mg, 0.40 mmol). The resulting mixture was stirred at room temperature for 2 hours and then concentrated under vacuum to give the crude product, which was purified by preparative high-performance liquid column chromatography: Welch Xtimate C18 150*30 mm*5 um to give the title compound N-(2-(3,3-dimethylazetidin-1-yl)ethyl)-6-methyl-5-((1-methyl-6-(pyrimidin-5-ylamino)-1H-pyrazolo[3,4-d]pyrimidin-3-yl)amino)nicotinamide (64 mg, 38%) as a yellow solid. LCMS (ESI): mass calcd. for C₂₄H₂₉N₁₁O, 487.56; m/z found, 488.4 [M+H]⁺. ¹H NMR (400 MHz, METHANOL-d₄) δ 9.28 (s, 2H), 8.97 (s, 1H), 8.81 (d, J=1.8 Hz, 1H), 8.74 (s, 1H), 8.44 (d, J=2.0 Hz, 1H), 3.85 (s, 3H), 3.40 (t, J=6.6 Hz, 2H), 3.11 (s, 4H), 2.73 (t, J=6.7 Hz, 2H), 2.62 (s, 3H), 1.23 (s, 6H)

### Example 56. N-(2-(4-azaspiro[2.5]octan-4-yl)ethyl)-6-methyl-5-((1-methyl-6-(pyrimidin-5-ylamino)-1H-pyrazolo[3,4-d]pyrimidin-3-yl)amino)nicotinamide

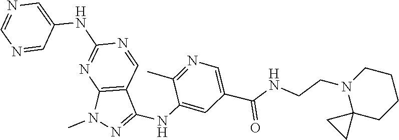 [SMILES: Cc1ncc(C(=O)NCCN2CCCCC23CC3)cc1Nc1nn(C)c2nc(Nc3cncnc3)ncc12]

### Step a: 4-azaspiro[2.5]octane

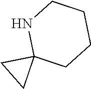

To a solution of 4-azaspiro[2.5]octan-5-one (1 g, 8.0 mmol) in tetrahydrofuran (80 mL) was added borane tetrahydrofuran complex solution (12 mL, 12 mmol) at 0° C. The resulting mixture was stirred at 70° C. for 16 hours before cooling to room temperature. The filtrate was concentrated to dryness under reduced pressure to afford the crude product 4-azaspiro [2.5]octane (1 g, 54%) as a yellow oil, which was used to the next step without further purification. LCMS (ESI): mass calcd. for C₇H₁₃N, 111.2; m/z found, 112.3 [M+H]⁺.

### Step b: 2-(4-azaspiro[2.5]octan-4-yl)acetonitrile

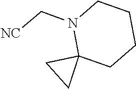 [SMILES: N#CCN1CCCCC12CC2]

To a solution of 4-azaspiro[2.5]octane (1 g, 1.6 mmol) and potassium carbonate (450 mg, 3.3 mmol) in N,N-dimethylformamide (12 mL) was added 2-bromoacetonitrile (234 mg, 1.9 mmol) at room-temperature. The resulting mixture was stirred at 60° C. for 16 h before cooling to room temperature. The resulting mixture was quenched with water (10 mL) and extracted with ethyl acetate (10 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=4:1) to give the title compound: 2-(4-azaspiro[2.5]octan-4-yl)acetonitrile (200 mg, 82%) as a yellow liquid. ¹H NMR (400 MHz, METHANOL-d₄) δ 3.38 (s, 2H), 2.65-2.57 (m, 2H), 1.37-1.28 (m, 2H), 1.25-1.15 (m, 2H), 1.03 (br d, J=5.1 Hz, 2H), 0.33-0.27 (m, 2H), 0.10-0.01 (m, 2H).

### Step c: 2-(4-azaspiro[2.5]octan-4-yl)ethanamine

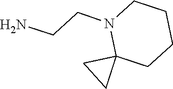 [SMILES: NCCN1CCCCC12CC2]

To a solution of 2-(4-azaspiro[2.5]octan-4-yl)acetonitrile (150 mg, 1.0 mmol) in THF (10 mL) was added lithium aluminium hydride (42 mg, 1.1 mmol) by portions at 0° C. (ice/water). The resultant mixture was stirred at 20° C. for 90 min before quenched with water (40 mg) at 0° C. The reaction mixture was filtered. And the filtrate was concentrated to dryness under reduced pressure to afford the crude product 2-(4-azaspiro[2.5]octan-4-yl)ethanamine (150 mg, 97%) as a colorless oil, which was used to the next step without further purification.

### Step d: N-(2-(4-azaspiro[2.5]octan-4-yl)ethyl)-6-methyl-5-((1-methyl-6-(pyrimidin-5-ylamino)-1H-pyrazolo[3,4-d]pyrimidin-3-yl)amino)nicotinamide

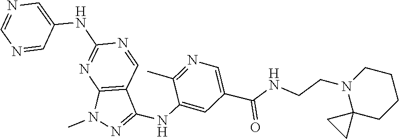 [SMILES: Cc1ncc(C(=O)NCCN2CCCCC23CC3)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino)nicotinic acid (100 mg, 0.27 mmol), HATU (121 mg, 0.32 mmol) and N,N-diisopropylethylamine (103 mg, 0.79 mmol) in N,N-dimethylformamide (4 mL) was added 2-(4-azaspiro[2.5]octan-4-yl)ethanamine (45 mg, 0.3 mmol). The mixture stirred at 25° C. for 2 h and then concentrated under vacuum to give the crude product, which was purified by preparative high-performance liquid column chromatography: Welch Xtimate C18 150*30 mm*5 um to give the title compound N-(2-(4-azaspiro[2.5]octan-4-yl)ethyl)-6-methyl-5-((1-methyl-6-(pyrimidin-5-ylamino)-1H-pyrazolo[3,4-d]pyrimidin-3-yl)amino)nicotinamide (30 mg, 22%) as a yellow solid. LCMS (ESI): mass calcd. for C₂₆H₃₁N₁₁O, 513.6; m/z found, 514.5 [M+H]⁺. ¹H NMR (400 MHz, METHANOL-d₄) δ 9.32 (s, 2H), 9.00 (s, 1H), 8.78-8.74 (m, 2H), 8.43 (d, J=1.8 Hz, 1H), 3.87 (s, 3H), 3.43 (br t, J=6.8 Hz, 2H), 3.03-2.95 (m, 4H), 2.64 (s, 3H), 1.73-1.67 (m, 2H), 1.57 (br s, 2H), 1.39 (br s, 2H), 0.61 (br s, 2H), 0.41 (br s, 2H).

### Example 57. N-(2-(4-azaspiro[2.4]heptan-4-yl)ethyl)-6-methyl-5-((1-methyl-6-(pyrimidin-5-ylamino)-1H-pyrazolo[3,4-d]pyrimidin-3-yl)amino)nicotinamide

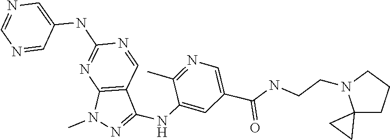 [SMILES: Cc1ncc(C(=O)NCCN2CCCC23CC3)cc1Nc1nn(C)c2nc(Nc3cncnc3)ncc12]

To a solution of 6-methyl-5-(2-(1-methyl-1H-pyrazol-4-yl)pyrazolo[5,1-b]thiazole-7-carboxamido)nicotinic acid (85 mg, 0.23 mmol), HATU (128 mg, 0.34 mmol) and N,N-diisopropylethylamine (87.3 mg, 0.67 mmol) in N,N-dimethylformamide (4 mL) was added 2-(4-azaspiro[2.4]heptan-4-yl)ethanamine (37.9 mg, 0.27 mmol). The mixture stirred at 25° C. for 2 h and then concentrated under vacuum to give the crude product, which was purified by preparative high-performance liquid column chromatography: Boston Green ODS 150*30 mm*5 um to give the title compound N-(2-(4-azaspiro[2.4]heptan-4-yl)ethyl)-6-methyl-5-((1-methyl-6-(pyrimidin-5-ylamino)-1H-pyrazolo[3,4-d]pyrimidin-3-yl)amino)nicotinamide (28.1 mg, 21%) as a yellow solid. LCMS (ESI): mass calcd. for C₂₅H₂₉N₁₁O₂, 499.571; m/z found, 500.3 [M+H]⁺. ¹H NMR (400 MHz, METHANOL-d₄) δ 9.32 (s, 2H), 9.01 (s, 1H), 8.83 (d, J=1.8 Hz, 1H), 8.78 (s, 1H), 8.48 (d, J=1.8 Hz, 1H), 3.88 (s, 3H), 3.68 (t, J=6.4 Hz, 2H), 3.51 (t, J=7.2 Hz, 2H), 3.06-3.01 (m, 2H), 2.66 (s, 3H), 2.22-2.13 (m, 2H), 2.10-2.04 (m, 2H), 1.24-1.18 (m, 2H), 0.86-0.80 (m, 2H).

### Example 58. N-(2-(5-azaspiro[2.4]heptan-5-yl)ethyl)-6-methyl-5-((1-methyl-6-(pyrimidin-5-ylamino)-1H-pyrazolo[3,4-d]pyrimidin-3-yl)amino)nicotinamide

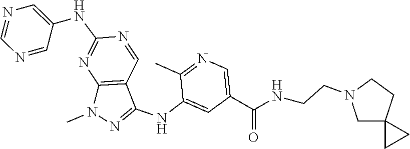 [SMILES: Cc1ncc(C(=O)NCCN2CCC3(CC3)C2)cc1Nc1nn(C)c2nc(Nc3cncnc3)ncc12]

### Step a: 2-(5-azaspiro[2.4]heptan-5-yl)acetonitrile

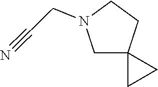

To a solution of 5-azaspiro[2.4]heptane (1.0 g, 7.5 mmol) and potassium carbonate (2.1 g, 15.0 mmol) in acetonitrile (20 mL) was added 2-bromoacetonitrile (1.1 g, 8.9 mmol) at room-temperature. The resulting mixture was stirred at 60° C. for 16 h before cooling to room temperature. The resulting mixture was quenched with water (10 mL) and extracted with ethyl acetate (10 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=3:2) to give the title compound 2-(5-azaspiro[2.4]heptan-5-yl)acetonitrile (480 mg, 47%) as a yellow oil.

### Step b: 2-(5-azaspiro[2.4]heptan-5-yl)ethanamine

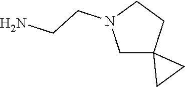

To a solution of 2-(5-azaspiro[2.4]heptan-5-yl)acetonitrile (450 mg, 3.3 mmol) in THF (4 mL) was added lithium aluminium hydride (138 mg, 3.6 mmol) by portions at 0° C. (ice/water). The resultant mixture was stirred at 20° C. for 90 min before quenched with water (137 mg) at 0° C. The reaction mixture was filtered. The filtrate was concentrated to dryness under reduced pressure to afford the crude product 2-(5-azaspiro[2.4]heptan-5-yl)ethanamine as a colorless oil. ¹H NMR (400 MHz, METHANOL-d₄) δ 2.83-2.66 (m, 4H), 2.64-2.44 (m, 4H), 1.82 (t, J=7.1 Hz, 2H), 0.62-0.49 (m, 4H).

### Step c: N-(2-(5-azaspiro[2.4]heptan-5-yl)ethyl)-6-methyl-5-((1-methyl-6-(pyrimidin-5-ylamino)-1H-pyrazolo[3,4-d]pyrimidin-3-yl)amino)nicotinamide

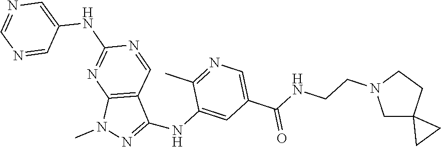 [SMILES: Cc1ncc(C(=O)NCCN2CCC3(CC3)C2)cc1Nc1nn(C)c2nc(Nc3cncnc3)ncc12]

To a solution of 6-methyl-5-(2-(1-methyl-1H-pyrazol-4-yl)pyrazolo[5,1-b]thiazole-7-carboxamido)nicotinic acid (120 mg, 0.32 mmol), HATU (181 mg, 0.48 mmol) and N,N-diisopropylethylamine (123 mg, 0.95 mmol) in N,N-dimethylformamide mL) was added 2-(5-azaspiro[2.4]heptan-5-yl)ethanamine (53.5 mg, 0.38 mmol). The mixture stirred at 25° C. for 2 h and then concentrated under vacuum to give the crude product, which was purified by preparative high-performance liquid column chromatography: Boston Green ODS 150*30 mm*5 um to give the title compound N-(2-(5-azaspiro[2.4]heptan-5-yl)ethyl)-6-methyl-5-((1-methyl-6-(pyrimidin-5-ylamino)-1H-pyrazolo[3,4-d]pyrimidin-3-yl)amino)nicotinamide (23.9 mg, 18%) as a yellow solid. LCMS (ESI): mass calcd. for C₂₅H₂₉N₁₁O, 499.571; m/z found, 500.3 [M+H]⁺. ¹H NMR (400 MHz, METHANOL-d₄) δ 8.86 (s, 2H), 8.60 (s, 1H), 8.55 (s, 1H), 8.36 (s, 1H), 8.20 (br s, 1H), 8.09 (s, 1H), 3.52-3.44 (m, 5H), 3.14 (br s, 2H), 3.09-2.95 (m, 3H), 2.29 (s, 3H), 1.78 (br t, J=7.3 Hz, 2H), 0.52-0.41 (m, 4H).

### Example 59. (R)-6-methyl-5-((1-methyl-6-(pyrimidin-5-ylamino)-1H pyrazolo-[3,4-d]pyrimidin-3-yl)amino)-N-(2-(3-methylpyrrolidin-1-yl)ethyl)nicotinamide

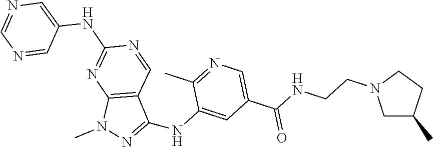 [SMILES: Cc1ncc(C(=O)NCCN2CC[C@@H](C)C2)cc1Nc1nn(C)c2nc(Nc3cncnc3)ncc12]

### Step a: (R)-2-(3-methylpyrrolidin-1-yl)acetonitrile

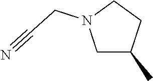

To a solution of (R)-3-methylpyrrolidine (0.70 g, 5.8 mmol) and potassium carbonate (1.6 g, 11.5 mmol) in acetonitrile (10 mL) was added 2-bromoacetonitrile (0.76 g, 6.3 mmol) at room-temperature. The resulting mixture was stirred at 60° C. for 12 h before cooling to room temperature. The reaction was concentrated under reduced pressure to give crude product, which was purified by column chromatography over silica gel (eluent: petroleum ether:ethyl acetate=7:3) to give the title compound (R)-2-(3-methylpyrrolidin-1-yl)acetonitrile (450 mg, 63%) as a pale yellow oil.

### Step b: (R)-2-(3-methylpyrrolidin-1-yl)ethanamine

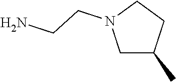

To a solution of (R)-2-(3-methylpyrrolidin-1-yl)acetonitrile (450 mg, 3.6 mmol) in THF (5 mL) was added lithium aluminium hydride (151 mg, 4.0 mmol) by portions at 0° C. (ice/water). The resultant mixture was stirred at 20° C. for 90 min before quenched with water (151 mg) at 0° C. The reaction mixture was filtered. And the filtrate was concentrated to dryness under reduced pressure to afford the crude product (R)-2-(3-methylpyrrolidin-1-yl)ethanamine as a colorless oil. ¹H NMR (400 MHz, METHANOL-d₄) δ 2.90-2.81 (m, 1H), 2.78-2.66 (m, 3H), 2.58-2.45 (m, 3H), 2.30-2.18 (m, 1H), 2.02 (td, J=7.7, 8.9 Hz, 2H), 1.41-1.29 (m, 1H), 1.02 (d, J=6.8 Hz, 3H).

### Step c: (R)-6-methyl-5-((1-methyl-6-(pyrimidin-5-ylamino)-1H-pyrazolo[3,4-d]pyrimidin-3-yl)amino)-N-(2-(3-methylpyrrolidin-1-yl)ethyl)nicotinamide

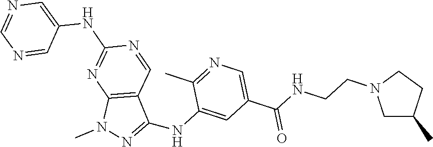 [SMILES: Cc1ncc(C(=O)NCCN2CC[C@@H](C)C2)cc1Nc1nn(C)c2nc(Nc3cncnc3)ncc12]

To a solution of 6-methyl-5-(2-(1-methyl-1H-pyrazol-4-yl)pyrazolo[5,1-b]thiazole-7-carboxamido)nicotinic acid (100 mg, 0.27 mmol), HATU (121 mg, 0.32 mmol) and N,N-diisopropylethylamine (103 mg, 0.79 mmol) in N,N-dimethylformamide (5 mL) was added (R)-2-(3-methylpyrrolidin-1-yl)ethanamine (37.4 mg, 0.29 mmol). The mixture stirred at 25° C. for 2 h and then concentrated under vacuum to give the crude product, which was purified by preparative high-performance liquid column chromatography: Phenomenex Gemini-NX C18 75*30 mm*3 um to give the title compound (R)-6-methyl-5-((1-methyl-6-(pyrimidin-5-ylamino)-1H-pyrazolo[3,4-d]pyrimidin-3-yl)amino)-N-(2-(3-methylpyrrolidin-1-yl)ethyl)nicotinamide (23.9 mg, 18%) as a yellow solid. LCMS (ESI): mass calcd. for C₂₄H₂₉N₁₁O, 487.56; m/z found, 488.2 [M+H]⁺. ¹H NMR (400 MHz, METHANOL-d₄) δ 9.27 (s, 2H), 8.97 (s, 1H), 8.81 (d, J=1.8 Hz, 1H), 8.73 (s, 1H), 8.44 (d, J=1.8 Hz, 1H), 3.84 (s, 3H), 3.60 (t, J=6.5 Hz, 2H), 3.20 (dd, J=7.5, 9.3 Hz, 1H), 3.09-3.01 (m, 1H), 3.00-2.91 (m, 3H), 2.62 (s, 3H), 2.44-2.31 (m, 2H), 2.16-2.07 (m, 1H), 1.54-1.44 (m, 1H), 1.07 (d, J=6.6 Hz, 3H).

### Example 60. (S)-4-methyl-5-((1-methyl-6-((1-methyl-1H-pyrazol-4-yl)amino)-1H-pyrazolo[3,4-d]pyrimidin-3-yl)amino)-N-(2-(2-methylpyrrolidin-1-yl)ethyl)thiophene-2-carboxamide

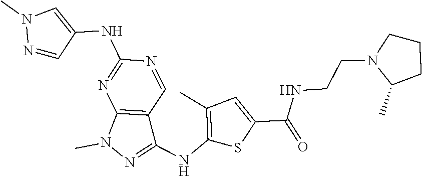 [SMILES: Cc1cc(C(=O)NCCN2CCC[C@@H]2C)sc1Nc1nn(C)c2nc(Nc3cnn(C)c3)ncc12]

To a mixture of methyl 4-methyl-5-((1-methyl-6-((1-methyl-1H-pyrazol-4-yl)amino)-1H-pyrazolo[3,4-d]pyrimidin-3-yl)amino)thiophene-2-carboxylate (150 mg, 0.32 mmol) and (S)-2-(2-methylpyrrolidin-1-yl)ethanamine (143 mg, 1.1 mmol) in THE (5 mL) was added AlMe₃ (0.64 mL, 1.3 mmol, 2M in toluene) under N₂ at 0° C. The resulting mixture was stirred at 60° C. for 15 h before cooled down room temperature. The reaction mixture was quenched with MeOH (50 mL) and filtered over celite. The filtrate was concentrated to the crude product. The crude was purified by preparative high-performance liquid column chromatography: Welch Xtimate C18 150*25 mm*5 um to give the title compound (S)-4-methyl-5-((1-methyl-6-((1-methyl-1H-pyrazol-4-yl)amino)-1H-pyrazolo[3,4-d]pyrimidin-3-yl)amino)-N-(2-(2-methylpyrrolidin-1-yl)ethyl)thiophene-2-carboxamide (22 mg, 12%) as a yellow solid. LCMS (ESI): mass calcd. for C₂₃H₃₀N₁₀OS, 494.2; m/z found, 495.2 [M+H]⁺.

¹H NMR (400 MHz, METHANOL-d₄) δ 8.84 (s, 1H), 8.10 (s, 1H), 7.66 (s, 1H), 7.39 (s, 1H), 3.92 (s, 3H), 3.85 (s, 3H), 3.61 (ddd, J=5.2, 8.2, 13.4 Hz, 1H), 3.52-3.36 (m, 2H), 3.15-3.06 (m, 1H), 2.52 (br s, 1H), 2.44-2.33 (m, 2H), 2.27 (s, 3H), 2.07-1.98 (m, 1H), 1.88-1.79 (m, 2H), 1.54-1.43 (m, 1H), 1.19 (d, J=6.2 Hz, 3H).

### Example 61. 4-methyl-5-((1-methyl-6-((1-methyl-1H-pyrazol-4-yl)amino) 1H-pyrazolo[3,4-d]pyrimidin-3-yl)amino)-N-(2-(tetrahydro-1H-furo[3,4-c]pyrrol-5(3H)-yl)ethyl)thiophene-2-carboxamide

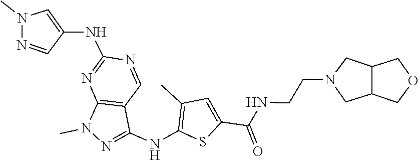 [SMILES: Cc1cc(C(=O)NCCN2CC3COCC3C2)sc1Nc1nn(C)c2nc(Nc3cnn(C)c3)ncc12]

To a mixture of methyl 4-methyl-5-((1-methyl-6-((1-methyl-1H-pyrazol-4-yl)amino)-1H-pyrazolo[3,4-d]pyrimidin-3-yl)amino)thiophene-2-carboxylate (150 mg, 0.32 mmol) and 2-(tetrahydro-1H-furo[3,4-c]pyrrol-5(3H)-yl)ethanamine (124 mg, 0.8 mmol) in THF (7 mL) was added Al Me₃ (0.48 mL, 0.96 mmol, 2M in toluene) under N₂ at 0° C. The resulting mixture was stirred at 60° C. for 15 h before cooled down room temperature. The reaction mixture was quenched with MeOH (30 mL) and filtered over celite. The filtrate was concentrated to the crude product. The crude was purified by preparative high-performance liquid column chromatography: Phenomenex Gemini-NX C18 75*30 mm*3 um to give the title compound 4-methyl-5-((1-methyl-6-((1-methyl-1H-pyrazol-4-yl)amino)-1H-pyrazolo[3,4-d]pyrimidin-3-yl)amino)-N-(2-(tetrahydro-1H-furo[3,4-c]pyrrol-5(3H)-yl)ethyl)thiophene-2-carboxamide (20 mg, 10%) as a yellow solid. LCMS (ESI): mass calcd. for C₂₄H₃₀N₁₀O₂S, 522.2; m/z found, 523.4 [M+H]⁺. ¹H NMR (400 MHz, METHANOL-d₄) δ 8.83 (s, 1H), 8.09 (s, 1H), 7.66 (s, 1H), 7.39 (s, 1H), 3.92 (s, 3H), 3.85 (s, 3H), 3.78-3.72 (m, 2H), 3.69-3.64 (m, 2H), 3.50 (t, J=6.7 Hz, 2H), 2.95-2.84 (m, 4H), 2.68 (t, J=6.6 Hz, 2H), 2.41 (br d, J=5.4 Hz, 2H), 2.27 (s, 3H).

### Example 62. N-(2-(1,4-oxazepan-4-yl)ethyl)-4-methyl-5-((1-methyl-6-((1-methyl-1H-pyrazol-4-yl)amino)-1H-pyrazolo[3,4-d]pyrimidin-3-yl)amino)thiophene-2-carboxamide

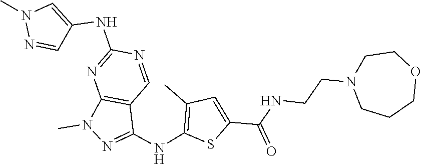 [SMILES: Cc1cc(C(=O)NCCN2CCCOCC2)sc1Nc1nn(C)c2nc(Nc3cnn(C)c3)ncc12]

To a solution of 2-(1,4-oxazepan-4-yl)ethanamine (138 mg, 0.95 mmol) in THF (7 mL) was added Trimethylaluminium (2.0 mol/1 in toluene, 0.64 mL, 1.27 mmol) slowly at 0° C. under N₂. The mixture was charged with N₂. The mixture was stirred at 0° C. for 30 min. Then a solution of ethyl 4-methyl-5-((1-methyl-6-((1-methyl-1H-pyrazol-4-yl)amino)-1H-pyrazolo[3,4-d]pyrimidin-3-yl)amino)thiophene-2-carboxylate (150 mg, 0.32 mmol) in THF (2 mL) was added slowly. The mixture was stirred at 60° C. for 12 hours. The mixture was cooled to 0° C. Water (1 mL) was added slowly and the mixture was filtered. The filtrate was concentrated to afford to give crude compound, which was purified by preparative high-performance liquid column chromatography: Phenomenex Gemini-NX C18 75*30 mm*3 um, then SFC column: DAICEL CHIRALCEL OJ-H (250 mm*30 mm, 10 um) to give the title compound N-(2-(1,4-oxazepan-4-yl)ethyl)-4-methyl-5-((1-methyl-6-((1-methyl-1H-pyrazol-4-yl)amino)-1H-pyrazolo[3,4-d]pyrimidin-3-yl)amino)thiophene-2-carboxamide (25 mg, 20%) as a yellow solid. LCMS (ESI): mass calcd. for C₂₃H₃₀N₁₀O₂S, 510.6; m/z found, 511.2 [M+H]⁺. ¹H NMR (400 MHz, METHANOL-d₄) δ 8.81 (s, 1H), 8.06 (s, 1H), 7.62 (s, 1H), 7.35 (s, 1H), 3.88 (s, 3H), 3.85-3.70 (m, 7H), 3.47 (t, J=6.7 Hz, 2H), 2.89-2.82 (m, 4H), 2.78 (t, J=6.7 Hz, 2H), 2.23 (s, 3H), 1.96-1.89 (m, 2H).

### Example 63. N-(2-(4,4-difluoropiperidin-1-yl)ethyl)-4-fluoro-3-((1-methyl-6-((1-methyl-1H-pyrazol-4-yl)amino)-1H-pyrazolo[3,4-d]pyrimidin-3-yl)amino)benzamide

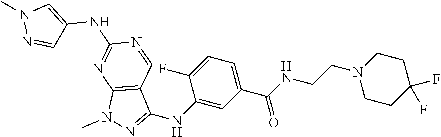 [SMILES: Cn1cc(Nc2ncc3c(Nc4cc(C(=O)NCCN5CCC(F)(F)CC5)ccc4F)nn(C)c3n2)cn1]

### Step a: ethyl 3-(2,4-dichloropyrimidine-5-carboxamido)-4-fluorobenzoate

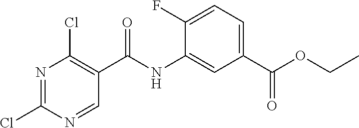 [SMILES: CCOC(=O)c1ccc(F)c(NC(=O)c2cnc(Cl)nc2Cl)c1]

To a solution of ethyl 3-amino-4-fluorobenzoate (3 g, 14.2 mmol) in dichloromethane (250 mL) was added 2,4-dichloropyrimidine-5-carbonyl chloride (2.3 g, 12.8 mmol). The mixture stirred at 25° C. for 12 h and then concentrated under vacuum to give the crude product, which was purified by column chromatography over silica gel (eluent: dichloromethane/methyl alcohol=9:1) to give the title compound ethyl 3-(2,4-dichloropyrimidine-5-carboxamido)-4-fluorobenzoate (2 g, 84%) as a yellow solid. LCMS (ESI): mass calcd. for C₁₄H₁₀C₁₂FN₃O₃, 358.15; m/z found, 358.0 [M+H]⁺.

### Step b: ethyl 3-(2-chloro-4-(1-methylhydrazinyl)pyrimidine-5-carboxamido)-4-fluorobenzoate

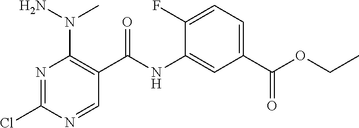 [SMILES: CCOC(=O)c1ccc(F)c(NC(=O)c2cnc(Cl)nc2N(C)N)c1]

To a solution of ethyl 3-(2,4-dichloropyrimidine-5-carboxamido)-4-fluorobenzoate (1 g, 4.6 mmol) and TEA (0.824 mL, 5.6 mmol) in THF (40 mL) was added methylhydrazine (1.02 g, 8.85 mmol). The mixture stirred at 25° C. for 2 h. The resulting mixture was quenched with water (30 mL) and extracted with ethyl acetate (30 mL×3). The combined organic phases were dried over anhydrous Na₂SO₄ and filtered. The filtrate was concentrated under reduced pressure to give the title compound ethyl 3-(2-chloro-4-(1-methylhydrazinyl)pyrimidine-5-carboxamido)-4-fluorobenzoate (1 g, 80.49%) as a red solid. LCMS (ESI): mass calcd. for C₁₅H₁₅ClFN₅O₃, 367.763; m/z found, 368.1[M+H]⁺.

### Step c: ethyl 3-((6-chloro-1-methyl-1H-pyrazolo[3,4-d]pyrimidin-3-yl)amino)-4-fluorobenzoate

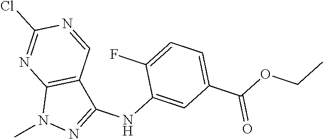 [SMILES: CCOC(=O)c1ccc(F)c(Nc2nn(C)c3nc(Cl)ncc23)c1]

To a solution of 3-(2-chloro-4-(1-methylhydrazinyl)pyrimidine-5-carboxamido)-4-fluorobenzoate (850 mg, 1.84 mmol) in toluene (15 mL) was added PCl₅ (392.3 mg, 1.88 mmol). The mixture stirred was at 120° C. for 12 h and then concentrated under vacuum to give the crude product, which was quenched with water (5 mL) and filtered. The filtrate was then concentrated to dryness under reduced pressure to give the title compound ethyl 3-((6-chloro-1-methyl-1H-pyrazolo[3,4-d]pyrimidin-3-yl)amino)-4-fluorobenzoate (780 mg, 71%) as a yellow solid. LCMS (ESI): mass calcd. for C₁₅H₁₃ClFN₈O₂, 349.74; m/z found, 350.1 [M+H]⁺.

### Step d: ethyl 4-fluoro-3-((1-methyl-6-((1-methyl-1H-pyrazol-4-yl)amino)-1H-pyrazolo[3,4-d]pyrimidin-3-yl)amino)benzoate

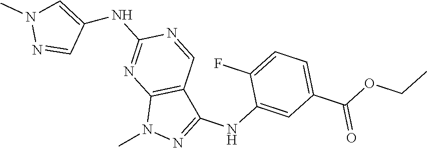 [SMILES: CCOC(=O)c1ccc(F)c(Nc2nn(C)c3nc(Nc4cnn(C)c4)ncc23)c1]

To a solution of ethyl 3-((6-chloro-1-methyl-1H-pyrazolo[3,4-d]pyrimidin-3-yl)amino)-4-fluorobenzoate (490 mg, 1.07 mmol), 1-methyl-1H-pyrazol-4-amine (124.8 mg, 1.3 mmol) and Cs₂CO₃ (1.04 g, 3.2 mmol) in N,N-dimethylformamide (10 mL) was added Brettphos-Pd-G3 (97 mg, 0.1 mmol) and Brettphos (114 mg, 0.2 mmol). The mixture was stirred at 80° C. for 12 h and then concentrated under vacuum to give the crude product, which was washed with MTBE (50 mL). The filtrate was then concentrated to dryness under reduced pressure to give the title compound ethyl 4-fluoro-3-((1-methyl-6-((1-methyl-1H-pyrazol-4-yl)amino)-1H-pyrazolo[3,4-d]pyrimidin-3-yl)amino)benzoate (490 mg, 93%) as a black solid. LCMS (ESI): mass calcd. for C₁₉H₁₉FN₈O₂, 410.405; m/z found, 411.2 [M+H]⁺.

### Step e: 4-fluoro-3-((1-methyl-6-((1-methyl-1H-pyrazol-4-yl)amino)-1H-pyrazolo[3,4-d]pyrimidin-3-yl)amino)benzoic acid

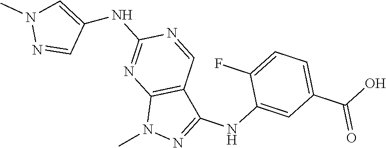 [SMILES: Cn1cc(Nc2ncc3c(Nc4cc(C(=O)O)ccc4F)nn(C)c3n2)cn1]

To a solution of ethyl 4-fluoro-3-((1-methyl-6-((1-methyl-1H-pyrazol-4-yl)amino)-1H-pyrazolo[3,4-d]pyrimidin-3-yl)amino)benzoate (490 mg, 1.07 mmol) in methanol/THF/H₂O=1:3:1 (10 mL) was added lithium hydroxide (38.7 mg, 1.6 mmol) at room temperature. The reaction mixture was stirred at 20° C. for 2 h. The mixture was adjusted to pH=3-4 with HCl (aq, 2 M). The mixture was filtered and washed with water (10 mL×3). The solid was evaporated under vacuum to give the desired product 4-fluoro-3-((1-methyl-6-((1-methyl-1H-pyrazol-4-yl)amino)-1H-pyrazolo[3,4-d]pyrimidin-3-yl)amino)benzoic acid (350 mg, 85%) as a white solid. LCMS (ESI): mass calcd. for C₁₇H₁₅FN₈O₂, 382.352; m/z found, 383.1 [M+H]⁺.

### Step f: N-(2-(4,4-difluoropiperidin-1-yl)ethyl)-4-fluoro-3-((1-methyl-6-((1-methyl-1H-pyrazol-4-yl)amino)-1H-pyrazolo[3,4-d]pyrimidin-3-yl)amino)benzamide

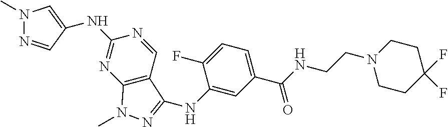 [SMILES: Cn1cc(Nc2ncc3c(Nc4cc(C(=O)NCCN5CCC(F)(F)CC5)ccc4F)nn(C)c3n2)cn1]

To a solution of 4-fluoro-3-((1-methyl-6-((1-methyl-1H-pyrazol-4-yl)amino)-1H-pyrazolo[3,4-d]pyrimidin-3-yl)amino)benzoic acid (100 mg, 0.18 mmol), HATU (136 mg, 0.36 mmol) and N,N-diisopropylethylamine (92 mg, 0.72 mmol) in N,N-dimethylformamide (2 mL) was added 2-(4, 4-difluoropiperidin-1-yl)ethanamine (30 mg, 0.18 mmol). The mixture stirred at 50° C. for 2 h and then concentrated under vacuum to give the crude product, which was purified by preparative high-performance liquid column chromatography: Boston prime C18 150*30 mm*5 um to give the title compound N-(2-(4,4-difluoropiperidin-1-yl)ethyl)-4-fluoro-3-((1-methyl-6-((1-methyl-1H-pyrazol-4-yl)amino)-1H-pyrazolo[3,4-d]pyrimidin-3-yl)amino)benzamide (35 mg, 34%) as a white solid. LCMS (ESI): mass calcd. for C₂₄H₂₇F₃N₁₀O, 528.533; m/z found, 529.3 [M+H]⁺. ¹H NMR (400 MHz, METHANOL-d₄) δ 8.93 (s, 1H), 8.73 (dd, J=8.11, 2.15 Hz, 1H), 8.11 (s, 1H), 7.66 (s, 1H), 7.38 (ddd, J=8.34, 4.47, 2.21 Hz, 1H), 7.23 (dd, J=11.09, 8.46 Hz, 1H), 3.93 (s, 3H), 3.89 (s, 3H), 3.54-3.59 (m, 2H), 2.68-2.72 (m, 6H), 2.00-2.08 (m, 4H).

### Example 64. N-(2-(3,3-dimethylazetidin-1-yl)ethyl)-4-methyl-3-((1-methyl-6-((1-methyl-1H-pyrazol-4-yl)amino)-1H-pyrazolo[3,4-d]pyrimidin-3-yl)amino)benzamide

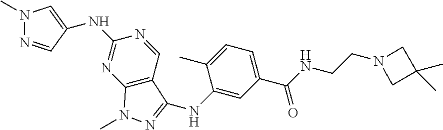

### Step a: ethyl 3-(2,4-dichloropyrimidine-5-carboxamido)-4-methylbenzoate

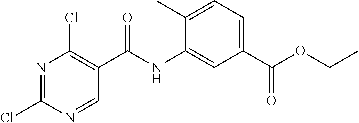 [SMILES: CCOC(=O)c1ccc(C)c(NC(=O)c2cnc(Cl)nc2Cl)c1]

To a solution of ethyl 3-amino-4-methylbenzoate (2.2 g, 13 mmol) in DCM (15 mL) was added 2,4-dichloropyrimidine-5-carbonyl chloride (3 g, 14.2 mmol) at room-temperature. The reaction mixture was stirred at room-temperature for 12 h. The resulting mixture was concentrated under reduced pressure to give crude product, which was purified by column chromatography over silica gel (eluent: dichloromethane/methyl alcohol=9:1) to give the title compound ethyl 3-(2,4-dichloropyrimidine-5-carboxamido)-4-methylbenzoate (2 g, 48%) as a yellow solid. LCMS (ESI): mass calcd. for C₅H₁₃Cl₂N₃O₃, 354.15; m/z found, 356.1 [M+H]⁺.

### Step b: ethyl 3-(2-chloro-4-(1-methylhydrazineyl)pyrimidine-5-carboxamido)-4-methylbenzoate

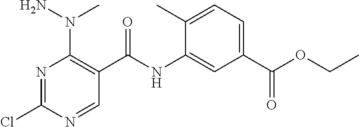 [SMILES: CCOC(=O)c1ccc(C)c(NC(=O)c2cnc(Cl)nc2N(C)N)c1]

To a solution of ethyl 3-(2,4-dichloropyrimidine-5-carboxamido)-4-methylbenzoate (1.8 g, 11 mmol) and triethylamine (1.28 g, 12.7 mmol) in THF (200 mL) was added methylhydrazine (2.7 g, 11 mmol). The resulting mixture was stirred at 25° C. for 1 hour and then concentrated under vacuum to give the crude product. The mixture was washed with water (100 mL×3). The solid was evaporated under vacuum to give desired product ethyl 3-(2-chloro-4-(1-methylhydrazineyl)pyrimidine-5-carboxamido)-4-methylbenzoate (1.8 g, 70.9%) as red solid. LCMS (ESI): mass calcd. for C₁₆H₁₈ClN₅O₃, 363.80; m/z found, 364.1 [M+H]⁺.

### Step c: ethyl 3-((6-chloro-1-methyl-1H-pyrazolo[3,4-d]pyrimidin-3-yl)amino)-4-methylbenzoate

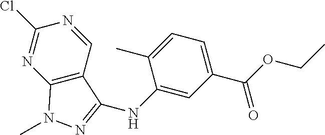 [SMILES: CCOC(=O)c1ccc(C)c(Nc2nn(C)c3nc(Cl)ncc23)c1]

To a solution of ethyl 3-(2-chloro-4-(1-methylhydrazineyl)pyrimidine-5-carboxamido)-4-methylbenzoate (1.7 g, 3.4 mmol) in toluene (200 mL) was added PCl₅(0.71 g, 3.4 mmol) at room-temperature. The reaction mixture was stirred at 120° C. for 12 h before cooling to room-temperature. The mixture was quenched by saturated aqueous NaHCO₃ (20 mL). The mixture was filtered and washed with water (10 mL×3). The solid was evaporated under vacuum to give desired product ethyl 3-((6-chloro-1-methyl-1H-pyrazolo[3,4-d]pyrimidin-3-yl)amino)-4-methylbenzoate (1.1 g, 93%) as yellow solid. LCMS (ESI): mass calcd. for C₁₆H₁₆ClN₈O₂, 345.79; m/z found, 346.1 [M+H]⁺.

### Step d: ethyl 4-methyl-3-((1-methyl-6-((1-methyl-1H-pyrazol-4-yl)amino)-1H-pyrazolo[3,4-d]pyrimidin-3-yl)amino)benzoate

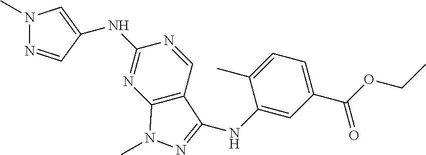 [SMILES: CCOC(=O)c1ccc(C)c(Nc2nn(C)c3nc(Nc4cnn(C)c4)ncc23)c1]

To a solution of ethyl 3-((6-chloro-1-methyl-1H-pyrazolo[3,4-d]pyrimidin-3-yl)amino)-4-methylbenzoate (0.9 g, 2.6 mmol), 1-methyl-1H-pyrazol-4-amine (0.3 g, 3.1 mmol) and Cs₂CO₃ (2.5 g, 7.8 mmol) in DMF (10 mL) was added Brettphos pd G3 (0.47 g, 0.5 mmol) and Brettphos (0.28 g, 0.5 mmol) at room-temperature. The reaction mixture was stirred at 80° C. for 3 h before cooling to room-temperature. The mixture was concentrated and washed with Tert-butyl ether (20 mL×3). The solid was evaporated under vacuum to give desired product ethyl 4-methyl-3-((1-methyl-6-((1-methyl-1H-pyrazol-4-yl)amino)-1H-pyrazolo[3,4-d]pyrimidin-3-yl)amino)-benzoate (800 mg, 55%) as yellow solid. LCMS (ESI): mass calcd. for C₂₀H₂₂N₈O₂, 406.45; m/z found, 407.2 [M+H]⁺.

### Step e: 4-methyl-3-((1-methyl-6-((1-methyl-1H-pyrazol-4-yl)amino)-1H-pyrazolo[3,4-d]pyrimidin-3-yl)amino)benzoic acid

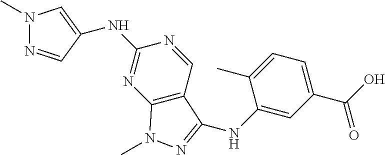 [SMILES: Cc1ccc(C(=O)O)cc1Nc1nn(C)c2nc(Nc3cnn(C)c3)ncc12]

To a solution of sodium hydrate (4 mL, 8 mmol, 2 M) in ethanol (4 mL) was added ethyl 4-methyl-3-((1-methyl-6-((1-methyl-1H-pyrazol-4-yl)amino)-1H-pyrazolo[3,4-d]pyrimidin-3-yl)amino)benzoate (790 mg, 2 mmol) at room-temperature. The reaction mixture was stirred at room-temperature for 1 h. The mixture was adjusted to pH=3-4 with HCl (aq, 2 M). The mixture was filtered and washed with water (20 mL×3). The solid was evaporated under vacuum to give desired product 4-methyl-3-((1-methyl-6-((1-methyl-1H-pyrazol-4-yl)amino)-1H-pyrazolo[3,4-d]pyrimidin-3-yl)amino)benzoic acid (620 mg, 84%) as black solid. LCMS (ESI): mass calcd. for C₁₈H₁₈N₈O₂, 378.4; m/z found, 379.2 [M+H]⁺.

### Step f: N-(2-(3,3-dimethylazetidin-1-yl)ethyl)-4-methyl-3-((1-methyl-6-((1-methyl-1H-pyrazol-4-yl)amino)-1H-pyrazolo[3,4-d]pyrimidin-3-yl)amino)benzamide

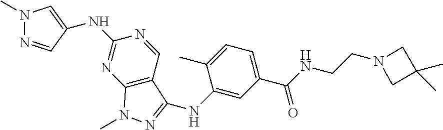

To a solution of 4-methyl-3-((1-methyl-6-((1-methyl-1H-pyrazol-4-yl)amino)-1H-pyrazolo[3,4-d]pyrimidin-3-yl)amino)benzoic acid (100 mg, 0.26 mmol), HATU (120 mg, 0.32 mmol) and N,N-diisopropylethylamine (102 mg, 0.79 mmol) in N,N-dimethylformamide (5 mL) was added 2-(3,3-dimethylazetidin-1-yl)ethan-1-amine (33 mg, 0.29 mmol). The mixture stirred at 20° C. for 2 h and then concentrated under vacuum to give the crude product, which was purified by preparative high-performance liquid column chromatography: Boston prime C18 150*30 mm*5 um to give the title compound N-(2-(3,3-dimethylazetidin-1-yl)ethyl)-4-methyl-3-((1-methyl-6-((1-methyl-1H-pyrazol-4-yl)amino)-1H-pyrazolo[3,4-d]pyrimidin-3-yl)amino)benzamide (28.7 mg, 21%) as a white solid. LCMS (ESI): mass calcd. for C₂₅H₃₂N₁₀O, 488.58; m/z found, 489.4 [M+H]⁺. ¹H NMR (400 MHz, METHANOL-d₄) δ 8.67 (s, 1H), 8.14 (d, J=1.5 Hz, 1H), 8.09 (s, 1H), 7.64 (s, 1H), 7.42 (dd, J=1.5, 7.9 Hz, 1H), 7.31 (d, J=7.9 Hz, 1H), 3.91 (s, 3H), 3.81 (s, 3H), 3.37 (t, J=6.7 Hz, 2H), 3.11 (s, 4H), 2.71 (t, J=6.6 Hz, 2H), 2.39 (s, 3H), 1.22 (s, 6H).

### Example 65. (S)-4-methyl-3-((1-methyl-6-((1-methyl-1H-pyrazol-4-yl)amino)-1H-pyrazolo[3,4-d]pyrimidin-3-yl)amino)-N-(2-(2-methylpyrrolidin-1-yl)ethyl)benzamide

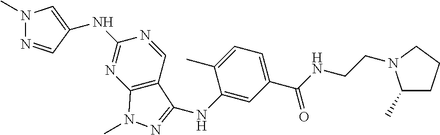 [SMILES: Cc1ccc(C(=O)NCCN2CCC[C@@H]2C)cc1Nc1nn(C)c2nc(Nc3cnn(C)c3)ncc12]

### Step a: (S)-2-(2-methylpyrrolidin-1-yl)acetonitrile

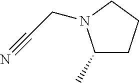

To a solution of (S)-2-methylpyrrolidine (3 g, 35 mmol) and potassium carbonate (9.7 g, 70 mmol) in N,N-dimethylformamide (40 mL) was added 2-bromoacetonitrile (4.6 g, 39 mmol) at room-temperature. The resulting mixture was stirred at 60° C. for 16 h before cooling to room temperature. The resulting mixture was quenched with water (45 mL) and extracted with ethyl acetate (100 mL*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=4:1) to give the title compound (S)-2-(2-methylpyrrolidin-1-yl)acetonitrile (3.5 g, 80%) as a yellow oil. ¹H NMR (400 MHz, METHANOL-d₄) δ 3.83-3.68 (m, 2H), 3.11-2.97 (m, 1H), 2.60-2.46 (m, 2H), 2.06-1.94 (m, 2H), 1.84-1.68 (m, 2H), 1.09 (d, J=6.0 Hz, 3H).

### Step b: (S)-2-(2-methylpyrrolidin-1-yl)ethanamine

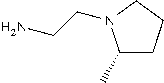

To a solution of 2-(4-azaspiro[2.4]heptan-4-yl)acetonitrile (3.5 g, 28 mmol) in THE (30 mL) was added lithium aluminium hydride (1.2 g, 31 mmol) by portions at 0° C. (ice/water). The resultant mixture was stirred at 20° C. for 90 min before quenched with water (100 mg) at 0° C. The reaction mixture was filtered. And the filtrate was concentrated to dryness under reduced pressure to afford the crude product (S)-2-(2-methylpyrrolidin-1-yl)ethanamine (2.5 g, 69%) as a colorless oil, which was used to the next step without further purification. ¹H NMR (400 MHz, METHANOL-d₄) δ 3.17-3.06 (m, 1H), 2.94-2.83 (m, 1H), 2.79-2.66 (m, 2H), 2.41-2.29 (m, 1H), 2.18-2.09 (m, 2H), 1.99-1.90 (m, 1H), 1.81-1.69 (m, 2H), 1.45-1.32 (m, 1H), 1.10 (d, J=6.0 Hz, 3H).

### Step c: (S)-4-methyl-3-((1-methyl-6-((1-methyl-1H-pyrazol-4-yl)amino)-1H-pyrazolo[3,4-d]pyrimidin-3-yl)amino)-N-(2-(2-methylpyrrolidin-1-yl)ethyl)benzamide

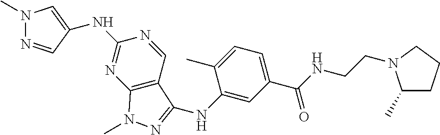 [SMILES: Cc1ccc(C(=O)NCCN2CCC[C@@H]2C)cc1Nc1nn(C)c2nc(Nc3cnn(C)c3)ncc12]

To a solution of 6-methyl-5-(2-(1-methyl-1H-pyrazol-4-yl)pyrazolo[5,1-b]thiazole-7-carboxamido)nicotinic acid (100 mg, 0.26 mmol), HATU (121 mg, 0.32 mmol) and N,N-diisopropylethylamine (102 mg, 0.79 mmol) in N,N-dimethylformamide (5 mL) was added (S)-2-(2-methylpyrrolidin-1-yl)ethanamine (37 mg, 0.29 mmol). The mixture was stirred at 25° C. for 2 h and then concentrated under vacuum to give the crude product, which was purified by preparative high-performance liquid column chromatography: Welch Xtimate C18 150*30 mm*5 um to give the title compound (S)-4-methyl-3-((1-methyl-6-((1-methyl-1H-pyrazol-4-yl)amino)-1H-pyrazolo[3,4-d]pyrimidin-3-yl)amino)-N-(2-(2-methylpyrrolidin-1-yl)ethyl)benzamide (31 mg, 24%) as a yellow solid. LCMS (ESI): mass calcd. for C₂₅H₃₂N₁₀O, 488.6; m/z found, 489.3 [M+H]⁺. ¹H NMR (400 MHz, METHANOL-d₄) δ 8.66 (s, 1H), 8.16 (d, J=1.5 Hz, 1H), 8.07 (s, 1H), 7.63 (s, 1H), 7.42 (dd, J=1.7, 7.8 Hz, 1H), 7.29 (d, J=7.9 Hz, 1H), 3.90 (s, 3H), 3.79 (s, 3H), 3.60 (ddd, J=5.2, 8.2, 13.3 Hz, 1H), 3.49-3.41 (m, 1H), 3.27-3.20 (m, 1H), 3.05 (td, J=7.7, 12.0 Hz, 1H), 2.47-2.41 (m, 1H), 2.39 (s, 3H), 2.36-2.23 (m, 2H), 2.02-1.92 (m, 1H), 1.82-1.72 (m, 2H), 1.47-1.36 (m, 1H), 1.12 (d, J=6.2 Hz, 3H).

### Example 66. N-(2-(2,2-dimethylpyrrolidin-1-yl)ethyl)-4-methyl-3-((1-methyl-6-((1-methyl-1H-pyrazol-4-yl)amino)-1H-pyrazolo[3,4-d]pyrimidin-3-yl)amino)benzamide

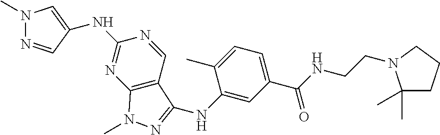 [SMILES: Cc1ccc(C(=O)NCCN2CCCC2(C)C)cc1Nc1nn(C)c2nc(Nc3cnn(C)c3)ncc12]

To a solution of 4-methyl-3-((1-methyl-6-((1-methyl-1H-pyrazol-4-yl)amino)-1H-pyrazolo[3,4-d]pyrimidin-3-yl)amino)benzoic acid (100 mg, 0.27 mmol), HATU (120 mg, 0.32 mmol) and N,N-diisopropylethylamine (102 mg, 0.8 mmol) in DMF (5 mL) was added 2-(2,2-dimethylpyrrolidin-1-yl)ethanamine (41.3 mg, 0.29 mmol). The resulting mixture was stirred at room temperature for 2 h and then concentrated under vacuum to give the crude product, which was purified by preparative high-performance liquid column chromatography: Welch Xtimate C18 150*30 mm*5 um to give the title compound N-(2-(2,2-dimethylpyrrolidin-1-yl)ethyl)-4-methyl-3-((1-methyl-6-((1-methyl-1H-pyrazol-4-yl)amino)-1H-pyrazolo[3,4-d]pyrimidin-3-yl)amino)benzamide (34.3 mg, 33%) as a yellow solid. LCMS (ESI): mass calcd. for C₂₆H₃₄N₁₀O, 502.6; m/z found, 503.2 [M+H]⁺. ¹H NMR (400 MHz, METHANOL-d₄) δ 8.66 (s, 1H), 8.14 (d, J=1.5 Hz, 1H), 8.08 (s, 1H), 7.64 (s, 1H), 7.41 (dd, J=1.7, 7.8 Hz, 1H), 7.30 (d, J=7.9 Hz, 1H), 3.90 (s, 3H), 3.80 (s, 3H), 3.47 (t, J=6.9 Hz, 2H), 2.91-2.83 (m, 2H), 2.67-2.58 (m, 2H), 2.39 (s, 3H), 1.86-1.75 (m, 2H), 1.70-1.62 (m, 2H), 1.01 (s, 6H).

### Example 67. N-(2-(5-azaspiro[2.4]heptan-5-yl)ethyl)-4-methyl-3-((1-methyl-6-((1-methyl-1H-pyrazol-4-yl)amino)-1H-pyrazolo[3,4-d]pyrimidin-3-yl)amino)benzamide

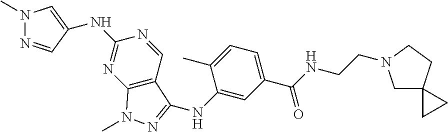 [SMILES: Cc1ccc(C(=O)NCCN2CCC3(CC3)C2)cc1Nc1nn(C)c2nc(Nc3cnn(C)c3)ncc12]

To a solution of 4-methyl-3-((1-methyl-6-((1-methyl-1H-pyrazol-4-yl)amino)-1H-pyrazolo[3,4-d]pyrimidin-3-yl)amino)benzoic acid (100 mg, 0.26 mmol), HATU (121 mg, 0.32 mmol) and N,N-diisopropylethylamine (102 mg, 0.79 mmol) in DMF (5 mL) was added 2-(5-azaspiro[2.4]heptan-5-yl)ethanamine (40.8 mg, 0.29 mmol). The resulting mixture was stirred at room temperature for 2 h and then concentrated under vacuum to give the crude product, which was purified by preparative high-performance liquid column chromatography: Welch Xtimate C18 150*30 mm*5 um to give the title compound N-(2-(5-azaspiro[2.4]heptan-5-yl)ethyl)-4-methyl-3-((1-methyl-6-((1-methyl-1H-pyrazol-4-yl)amino)-1H-pyrazolo[3,4-d]pyrimidin-3-yl)amino)benzamide (41.2 mg, 30%) as a yellow solid. LCMS (ESI): mass calcd. for C₂₆H₃₂N₁₀O, 500.6; m/z found, 501.2 [M+H]⁺. ¹H NMR (400 MHz, METHANOL-d₄) δ 8.66 (s, 1H), 8.16 (d, J=1.3 Hz, 1H), 8.08 (s, 1H), 7.63 (s, 1H), 7.43 (dd, J=1.5, 7.7 Hz, 1H), 7.30 (d, J=7.9 Hz, 1H), 3.90 (s, 3H), 3.80 (s, 3H), 3.52 (t, J=6.8 Hz, 2H), 2.83 (t, J=7.1 Hz, 2H), 2.73 (t, J=6.7 Hz, 2H), 2.61 (s, 2H), 2.39 (s, 3H), 1.84 (t, J=7.1 Hz, 2H), 0.63-0.54 (m, 4H).

### Example 68. N-(2-((2S,6R)-2,6-dimethylpiperidin-1-yl)ethyl)-6-methyl-5-((1-methyl-6-((1-methyl-1H-pyrazol-4-yl)amino)-1H-pyrazolo[4,3-c]pyridin-3-yl)amino)nicotinamide

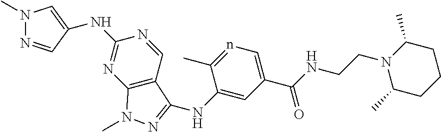

### Step a: 6-chloro-3-iodo-1H-pyrazolo[4,3-c]pyridine

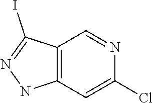 [SMILES: Clc1cc2[nH]nc(I)c2cn1]

To a solution of 6-chloro-1H-pyrazolo[4,3-c]pyridine (1.2 g, 7.8 mmol) in DMF (15 mL) was added NIS (2.6 g, 11.7 mmol). The reaction was stirred at 80° C. for 1 h. The reaction mixture was washed with 10 mL of saturated aqueous Na₂SO₃. The resulting solution was added saturated aqueous NaHCO₃ (20 mL) and extracted with ethyl acetate (20 mL×3). The combined organic layer was dried over Na₂SO₄, filtered and evaporated in vacuum to give the crude compound which was purified by silica gel column chromatography (eluent: petroleum ether/ethyl acetate=100:0 to 50:50) to give the title compound 6-chloro-3-iodo-1H-pyrazolo[4,3-c]pyridine (3 g, 60%) as a yellow solid. LCMS (ESI): mass calcd. for C₆H₃ClIN₃, 279.466; m/z found, 279.9[M+H]⁺.

### Step b: 6-chloro-3-iodo-1-methyl-1H-pyrazolo[4,3-c]pyridine

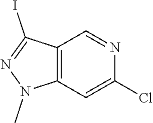

To a solution of 6-chloro-3-iodo-1H-pyrazolo[4,3-c]pyridine (3 g, 14.7 mmol) in THF (50 mL) was added NaH (284 mg, 7.1 mmol, 60%) by portions at 0° C. (ice/water). The resultant mixture was stirred at 0° C. for 30 minutes. Then iodomethane (1.2 mL, 23.6 mmol) was added to the mixture and the mixture was stirred at 0° C. for 2 h. The reaction mixture was quenched with water (50 mL) slowly and extracted with ethyl acetate (50 mL×3). The combined organic layer was dried over Na₂SO₄, filtered and evaporated in vacuum to afford crude compound which was purified by silica gel column chromatography (eluent: petroleum ether/ethyl acetate=100:0 to 50:50). The desired fractions were collected and the solvent was concentrated to dryness under vacuum to afford the crude compound which was purified by preparative high-performance liquid column chromatography: Boston Uni C18 40*150 mm*5 um to give the title compound 6-chloro-3-iodo-1-methyl-1H-pyrazolo[4,3-c]pyridine (1.2 g, 87%) as a white solid. LCMS (ESI): mass calcd. for C₇H₅ClIN₃, 293.492; m/z found, 293.9[M+H]⁺.

### Step c: 5-amino-6-methylnicotinic acid

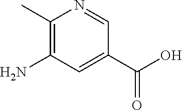

To a solution of ethyl 5-amino-6-methylnicotinate (500 mg, 2.8 mmol) in THF (1.2 mL), methanol (1.2 mL) and H₂O (0.5 mL) was added LiOH (122 mg, 3.1 mmol). The mixture stirred at 50° C. for 2 h. Solvent was consumed under reduced pressure to give crude compound which was diluted with water (2.5 mL) and acidified by 2N HCl to pH=3-4. The solid was precipitated and collected by filtrate. The solid was dried in high vacuo to give the title compound 5-amino-6-methylnicotinic acid (420 mg, 99%) as a yellow solid.

### Step d: 5-amino-N-(2-((2S,6R)-2,6-dimethylpiperidin-1-yl)ethyl)-6-methylnicotinamide

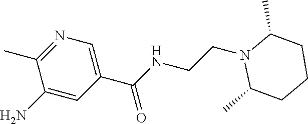 [SMILES: Cc1ncc(C(=O)NCCN2[C@H](C)CCC[C@@H]2C)cc1N]

To a solution of 5-amino-6-methylnicotinic acid (400 mg, 2.6 mmol), HATU (1.2 g, 3.2 mmol) and N,N-diisopropylethylamine (1.2 g, 3.2 mmol) in N,N-dimethylformamide (40 mL) was added 2-((2S,6R)-2,6-dimethylpiperidin-1-yl)ethanamine (507 mg, 2.6 mmol). The mixture stirred at 25° C. for 2 h and then concentrated under vacuum to give the crude product, which was purified by preparative high-performance liquid column chromatography: Boston Green ODS 150*30 mm*5 um to give the title compound 5-amino-N-(2-((2S,6R)-2,6-dimethylpiperidin-1-yl)ethyl)-6-methylnicotinamide (720 mg, 94%) as a yellow solid. LCMS (ESI): mass calcd. for C₁₆H₂₆N₄O, 290.404; m/z found, 291.3 [M+H]⁺.

### Step e: 5-((6-chloro-1-methyl-1H-pyrazolo[4,3-c]pyridin-3-yl)amino)-N-(2-((2S,6R)-2,6-dimethylpiperidin-1-yl)ethyl)-6-methylnicotinamide

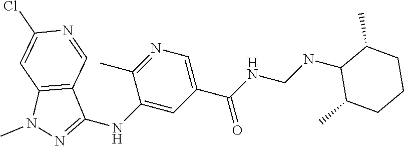 [SMILES: Cc1ncc(C(=O)NCNC2[C@H](C)CCC[C@@H]2C)cc1Nc1nn(C)c2cc(Cl)ncc12]

To a solution of 6-chloro-3-iodo-1-methyl-1H-pyrazolo[4,3-c]pyridine (480 mg, 1.6 mmol), 5-amino-N-(2-((2S,6R)-2,6-dimethylpiperidin-1-yl)ethyl)-6-methylnicotinamide (475 mg, 1.6 mmol) and Cs₂CO₃ (1.6 g, 4.9 mmol) in N,N-dimethylformamide (10 mL) was added Brettphos-Pd-G₃ (297 mg, 0.33 mmol) and Brettphos (176 mg, 0.33 mmol). The mixture stirred at 90° C. for 12 h and then concentrated under vacuum to give the crude product, which was purified by preparative high-performance liquid column chromatography: Phenomenex Gemini-NX C18 75*30 mm*3 um to give the title compound 5-((6-chloro-1-methyl-1H-pyrazolo[4,3-c]pyridin-3-yl)amino)-N-(2-((2S,6R)-2,6-dimethylpiperidin-1-yl)ethyl)-6-methylnicotinamide (100 mg, 13%) as a yellow solid. LCMS (ESI): mass calcd. for C₂₃H₃₀ClN₇O, 455.984; m/z found, 456.2 [M+H]⁺.

### Step f: N-(2-((2S,6R)-2,6-dimethylpiperidin-1-yl)ethyl)-6-methyl-5-((1-methyl-6-((1-methyl-1H-pyrazol-4-yl)amino)-1H-pyrazolo[4,3-c]pyridin-3-yl)amino)nicotinamide

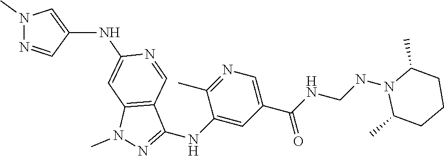 [SMILES: Cc1ncc(C(=O)NCNN2[C@H](C)CCC[C@@H]2C)cc1Nc1nn(C)c2cc(Nc3cnn(C)c3)ncc12]

To a solution of 5-((6-chloro-1-methyl-1H-pyrazolo[4,3-c]pyridin-3-yl)amino)-N-(2-((2S,6R)-2,6-dimethylpiperidin-1-yl)ethyl)-6-methylnicotinamide (100 mg, 0.22 mmol), 1-methylpyrazol-4-amine (32 mg, 0.33 mmol) and Cs₂CO₃ (214 mg, 0.66 mmol) in N,N-dimethylformamide (10 mL) was added Brettphos-Pd-G₃ (40 mg, 0.04 mmol) and Brettphos (24 mg, 0.04 mmol). The mixture stirred at 100° C. for 12 h and then concentrated under vacuum to give the crude product, which was purified by preparative high-performance liquid column chromatography: Phenomenex Gemini-NX C18 75*30 mm*3 um to give the title compound N-(2-((2S,6R)-2,6-dimethylpiperidin-1-yl)ethyl)-6-methyl-5-((1-methyl-6-((1-methyl-1H-pyrazol-4-yl)amino)-1H-pyrazolo[4,3-c]pyridin-3-yl)amino)nicotinamide (55.6 mg, 40%) as a yellow solid. LCMS (ESI): mass calcd. for C₂₇H₃₆N₁₀O, 516.641; m/z found, 517.3 [M+H]⁺. ¹H NMR (400 MHz, DMSO-d6) δ 8.70 (s, 2H), 8.48-8.54 (m, 2H), 8.21 (d, J=3.26 Hz, 2H), 7.86 (s, 1H), 7.44 (s, 1H), 6.35 (s, 1H), 3.82 (s, 3H), 3.64-3.75 (m, 3H), 3.33 (br d, J=7.53 Hz, 2H), 2.83 (br t, J=7.53 Hz, 2H), 2.67 (br s, 2H), 2.60 (s, 3H), 1.51-1.63 (m, 3H), 1.24 (br s, 3H), 1.15 (d, J=6.02 Hz, 6H).

### Example 69. N-(2-(2,2-dimethylpyrrolidin-1-yl)ethyl)-6-methyl-5-((1-methyl-6-((1-methyl-1H-pyrazol-4-yl)amino)-1H-pyrazolo[4,3-c]pyridin-3-yl)amino)nicotinamide

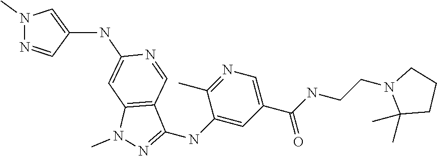 [SMILES: Cc1ncc(C(=O)NCCN2CCCC2(C)C)cc1Nc1nn(C)c2cc(Nc3cnn(C)c3)ncc12]

To a solution of 5-((6-chloro-1-methyl-1H-pyrazolo[3,4-d]pyrimidin-3-yl) amino)-N-(2-(2,2-dimethylpyrroli-din-1-yl)ethyl)-6-methylnicotinamide (70 mg, 0.16 mmol) dissolved in DMF (7 mL) was added 1-methyl-1H-pyrazol-4-amine (23 mg 0.24 mmol), Then Cs₂CO₃ (155 mg 0.48 mmol), Brettphos (17 mg 0.03 mmol) and Brettphos-Pd-G3 (28 mg 0.03 mmol) was added. The mixture was charged with N₂. The mixture was stirred at 110° C. for 12 hours. The mixture was cooled to room temperature and filtered. The filtrate was concentrated to afford crude compound. The crude compound was purified by preparative high-performance liquid column chromatography: Phenomenex Gemini-NX C18 75*30 mm*3 um to give the title compound N-(2-(2,2-dimethylpyrrolidin-1-yl)ethyl)-6-methyl-5-((1-methyl-6-((1-methyl-1H-pyrazol-4-yl)amino)-1H-pyrazolo[4,3-c]pyridin-3-yl)amino)nicotinamide (20.9 mg, 25%) as brown powder. LCMS (ESI): mass calcd. for C₂₆H₃₄N₁₀O, 502.6; m/z found, 503.2 [M+H]⁺. ¹H NMR (400 MHz, DMSO-d₆) δ 8.71 (s, 1H), 8.64 (br s, 1H), 8.53 (s, 2H), 8.47 (s, 1H), 8.23 (br s, 2H), 7.86 (s, 1H), 7.44 (s, 1H), 6.35 (s, 1H), 3.82-3.83 (m, 3H), 3.66-3.75 (m, 2H), 3.40 (br d, J=5.48 Hz, 2H), 2.96 (br t, J=7.03 Hz, 2H), 2.69 (br t, J=6.26 Hz, 2H), 2.58-2.63 (m, 3H), 1.70-1.80 (m, 2H), 1.65 (br d, J=7.75 Hz, 2H), 1.02 (s, 6H).

### Example 70. 2-(2,2-dimethylpyrrolidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

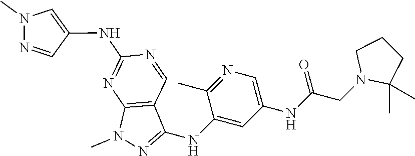

### Step a: 2,4-dichloro-N-(2-methyl-5-nitropyridin-3-yl)pyrimidine-5-carboxamide

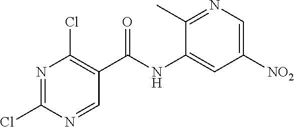 [SMILES: Cc1ncc([N+](=O)[O-])cc1NC(=O)c1cnc(Cl)nc1Cl]

To a solution of 2-methyl-5-nitropyridin-3-amine 2,4-dichloropyrimidine-5-carbonyl chloride (3.3 g, 21.3 mmol) in dioxane (150 mL) was added 2,4-dichloropyrimidine-5-carbonyl chloride (5 g, 23.6 mmol) at 0° C. The resulting mixture was stirred at 80° C. for 12 h before cooling to room-temperature. The reaction mixture was concentrated under reduced pressure to give crude product, which was triturated with TBME/EtOAc (3/1, 120 mL) to give the title compound 2,4-dichloro-N-(2-methyl-5-nitropyridin-3-yl)pyrimidine-5-carboxamide (6.7 g, 44%) as a gray solid. LCMS (ESI): mass calcd. for C₁₁H₇C₁₂N₅O₃, 327.0; m/z found, 328.0 [M+H]⁺.

### Step b: 2-chloro-N-(2-methyl-5-nitropyridin-3-yl)-4-(1-methylhydrazinyl)pyrimidine-5-carboxamide

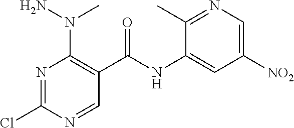 [SMILES: Cc1ncc([N+](=O)[O-])cc1NC(=O)c1cnc(Cl)nc1N(C)N]

To a solution of 2,4-dichloro-N-(2-methyl-5-nitropyridin-3-yl)pyrimidine-5-carboxamide (6.7 g, 17.5 mmol) in THE (250 mL) was added methylhydrazine (2.51 g, 21.8 mmol, 40% in H₂O) and Et₃N (6.09 mL, 43.7 mmol) at 0° C. The resulting mixture was stirred at room temperature for 1 h. The mixture was quenched by the addition of HCl aqueous (1N aqueous in water) (10 mL). The mixture was extracted with EtOAc (70 mL*3). The combined organic layers were washed with brine (30 mL*2), dried over anhydrous Na₂SO₄, filtered and concentrated under vacuum to afford 2-chloro-N-(2-methyl-5-nitropyridin-3-yl)-4-(1-methylhydrazinyl)pyrimidine-5-carboxamide (6.5 g, crude) as a yellow solid. LCMS (ESI): mass calcd. for C₁₂H₁₂ClN₇O₃, 337.1; m/z found, 338.1[M+H]⁺. ¹H NMR (400 MHz, DMSO-d₆) δ 9.99 (s, 1H), 9.04 (d, J=2.4 Hz, 1H), 8.88 (d, J=2.2 Hz, 1H), 8.12 (s, 1H), 5.11 (s, 2H), 3.27 (s, 3H), 2.61 (s, 3H).

### Step c: 6-chloro-1-methyl-N-(2-methyl-5-nitropyridin-3-yl)-1H-pyrazolo[3,4-d]pyrimidin-3-amine

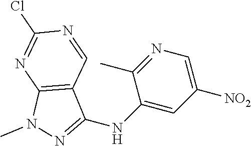 [SMILES: Cc1ncc([N+](=O)[O-])cc1Nc1nn(C)c2nc(Cl)ncc12]

To a solution of 2-chloro-N-(2-methyl-5-nitropyridin-3-yl)-4-(1-methylhydrazinyl)pyrimidine-5-carboxamide (3 g, 8.53 mmol) in toluene (300 mL) was added PCl₅ (1.78 g, 8.53 mmol) at room temperature. The reaction mixture was stirred at 100° C. for 2 h before cooling to room-temperature. The reaction mixture was evaporated to give crude. The crude was purified by flash column chromatography over a 4 g silica gel (gradient: Petroleum ether/ethyl acetate=0:100 to 100:0 then ethyl acetate/methanol=70:30). The desired fractions were collected and the solvent was concentrated under vacuum to afford product 6-chloro-1-methyl-N-(2-methyl-5-nitropyridin-3-yl)-1H-pyrazolo[3,4-d]pyrimidin-3-amine (2.8 g, 28%) as a yellow solid. LCMS (ESI): mass calcd. for C₁₂H₁₀ClN₇O₂, 319.1; m/z found, 319.7 [M+H]⁺.

### Step d: 1-methyl-N⁶-(1-methyl-1H-pyrazol-4-yl)-N³-(2-methyl-5-nitropyridin-3-yl)-1H-pyrazolo[3,4-d]pyrimidine-3,6-diamine

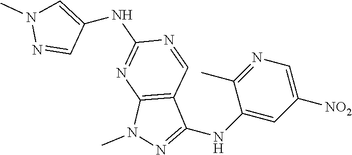 [SMILES: Cc1ncc([N+](=O)[O-])cc1Nc1nn(C)c2nc(Nc3cnn(C)c3)ncc12]

To a mixture of 6-chloro-1-methyl-N-(2-methyl-5-nitropyridin-3-yl)-1H-pyrazolo[3,4-d]pyrimidin-3-amine (2.8 g, 8.76 mmol), 1-methyl-1H-pyrazol-4-amine (1.02 g 10.5 mmol), Brettphos-Pd-G3 (794 mg, 0.88 mmol) and Brettphos (940 mg, 1.75 mmol) in DMF (150 mL) was added Cs₂CO₃ (8.56 g, 26.3 mmol) and Molecular sieves pack 4 A powder (500 mg) at room temperature. The reaction mixture was purged with N₂ for 2 minutes. Then the reaction mixture was stirred at 90° C. for 2 h under N₂. The reaction mixture was cooled down room temperature. The reaction mixture was filtered. The filtrate was evaporated to give crude. The crude was purified by flash column chromatography over a 40 g silica gel (gradient: Petroleum ether/ethyl acetate=0/100 to 100/0 then ethyl acetate/methanol=85/15). The desired fractions were collected and the solvent was concentrated under vacuum to afford product 1-methyl-N⁶-(1-methyl-1H-pyrazol-4-yl)-N³-(2-methyl-5-nitropyridin-3-yl)-1H-pyrazolo[3,4-d]pyrimidine-3,6-diamine (1.1 g, 13%) as a yellow solid. LCMS (ESI): mass calcd. for C₁₆H₁₆N₁₀O₂, 380.1; m/z found, 381.2 [M+H]⁺.

### Step e: N³-(5-amino-2-methylpyridin-3-yl)-1-methyl-N⁶-(1-methyl-1H-pyrazol-4-yl)-1H-pyrazolo[3,4-d]pyrimidine-3,6-diamine

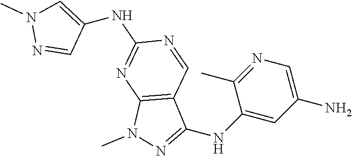 [SMILES: Cc1ncc(N)cc1Nc1nn(C)c2nc(Nc3cnn(C)c3)ncc12]

To a mixture of 1-methyl-N₆-(1-methyl-1H-pyrazol-4-yl)-N₃-(2-methyl-5-nitropyridin-3-yl)-1H-pyrazolo[3,4-d]pyrimidine-3,6-diamine (1.6 g, 4.2 mmol) in AcOH (50 mL) was added Fe (1.2 g, 21 mmol) at room temperature. The reaction mixture was stirred at 50° C. for 5 hours. The reaction mixture was filtered. The filtrate was concentrated to give crude as black oil. The crude product was triturated with methanol (10 mL) and filtered. The filtrate was concentrated and purified by column on silica gel (eluent: dichloromethane:methanol=95:5). The pure fractions were collected and the solvent was evaporated to afford product N³-(5-amino-2-methylpyridin-3-yl)-1-methyl-N⁶-(1-methyl-1H-pyrazol-4-yl)-1H-pyrazolo[3,4-d]pyrimidine-3,6-diamine (400 mg, 20%) as chocolate solid. LCMS (ESI): mass calcd. for C₁₆H₁₈N₁₀, 350.2; m/z found, 351.2 [M+H]⁺.

### Step f: 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

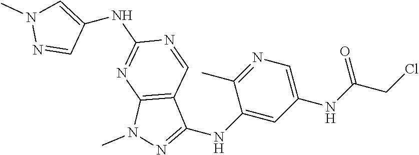 [SMILES: Cc1ncc(NC(=O)CCl)cc1Nc1nn(C)c2nc(Nc3cnn(C)c3)ncc12]

To a mixture of N³-(5-amino-2-methylpyridin-3-yl)-1-methyl-N⁶-(1-methyl-1H-pyrazol-4-yl)-1H-pyrazolo[3,4-d]pyrimidine-3,6-diamine (670 mg, 67% purity, 1.4 mmol) and NaHCO₃ (177 mg, 2.1 mmoL) in DMF (35 mL) was added 2-chloroacetyl chloride (167 mg, 1.5 mmol) dropwise at 0° C. After the reaction was warmed to room temperature with stirred for 2 hours. The reaction mixture was filtered. The filtrate was concentrated in vacuo to give crude. The crude was purified by flash column chromatography over a 12 g silica gel (gradient: DCM:MeOH from 100:0 to 85:15). The desired fractions were collected and the solvent was concentrated under vacuum to afford product 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (320 mg, 60%) as a pale brown solid. LCMS (ESI): mass calcd. for C₁₅H₁₉ClN₁₀O, 426.1; m/z found, 427.3 [M+H]⁺.

### Step g: 2-(2,2-dimethylpyrrolidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

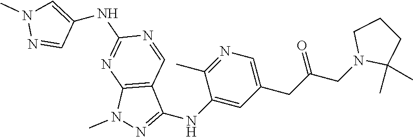

To a mixture of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (60 mg, 0.14 mmol), K₂CO₃ (58 mg, 0.42 mmoL) and NaI (13 mg, 0.08 mmol) in DMF (4 mL) was added 2,2-dimethylpyrrolidine (17 mg, 0.17 mmol). The reaction mixture was stirred at 50° C. for 2 h before cooling to room-temperature. The reaction mixture was filtered and the filtrate was concentrated under vacuum to give crude product, which was purified by preparative by preparative high-performance liquid chromatography over Column: Phenomenex Gemini-NX 80*40 mm*3 um to give the title compound 2-(2,2-dimethylpyrrolidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (32 mg, 44%) as a white solid. LCMS (ESI): mass calcd. for C₂₄H₃₁N₁₁O, 489.3; m/z found, 490.1 [M+H]⁺. ¹H NMR (400 MHz, METHANOL-d₄) δ 8.85 (s, 1H), 8.70 (d, J=2.1 Hz, 1H), 8.32 (d, J=2.1 Hz, 1H), 8.11 (s, 1H), 7.66 (s, 1H), 3.92 (s, 3H), 3.85 (s, 3H), 3.26 (s, 2H), 2.91 (t, J=7.2 Hz, 2H), 2.56 (s, 3H), 1.91-1.84 (m, 2H), 1.82-1.76 (m, 2H), 1.11 (s, 6H).

### Example 71. (S)—N-(6-methyl-5-((1-methyl-6-((1-methyl-1H-pyrazol-4-yl)amino)-1H-pyrazolo[3,4-d]pyrimidin-3-yl)amino)pyridin-3-yl)-2-(2-methylpyrrolidin-1-yl)acetamide

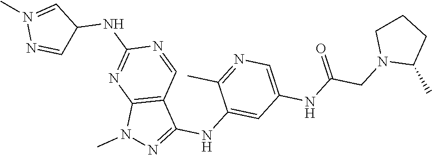

To a mixture of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (70 mg, 0.16 mmol), K₂CO₃ (68 mg, 0.49 mmoL) and NaI (27 mg, 0.18 mmol) in DMF (6 mL) was added (S)-2-methylpyrrolidine (42 mg, 0.49 mmol). The reaction mixture was stirred at 50° C. for 2 h before cooling to room-temperature. The reaction mixture was filtered and the filtrate was concentrated under vacuum to give crude product, which was purified by preparative high-performance liquid chromatography over Column: Phenomenex Gemini-NX 80*40 mm*3 um to give the title compound (S)—N-(6-methyl-5-((1-methyl-6-((1-methyl-1H-pyrazol-4-yl)amino)-1H-pyrazolo[3,4-d]pyrimidin-3-yl)amino)pyridin-3-yl)-2-(2-methylpyrrolidin-1-yl)acetamide (56 mg, 67%) as an off-white solid. LCMS (ESI): mass calcd. for C₂₃H₂₉N₁₁O, 475.3; m/z found, 476.2 [M+H]⁺. ¹H NMR (400 MHz, METHANOL-d₄) δ 8.85 (s, 1H), 8.68 (d, J=2.0 Hz, 1H), 8.35 (d, J=2.1 Hz, 1H), 8.11 (s, 1H), 7.67 (s, 1H), 3.93 (s, 3H), 3.86 (s, 3H), 3.56 (d, J=16.0 Hz, 1H), 3.29-3.24 (m, 1H), 3.10 (d, J=15.9 Hz, 1H), 2.64-2.58 (m, 1H), 2.56 (s, 3H), 2.41 (q, J=8.8 Hz, 1H), 2.08-1.97 (m, 1H), 1.93-1.72 (m, 2H), 1.59-1.45 (m, 1H), 1.17 (d, J=6.1 Hz, 3H).

### Example 72. 2-(4,4-difluoropiperidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

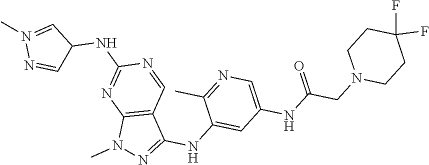

To a mixture of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (70 mg, 0.16 mmol), K₂CO₃ (68 mg, 0.49 mmoL) and NaI (27 mg, 0.18 mmol) in DMF (6 mL) was added 4,4-difluoropiperidine (60 mg, 0.49 mmol). The reaction mixture was stirred at 50° C. for 1.5 h before cooling to room-temperature. The reaction mixture was filtered and the filtrate was concentrated under vacuum to give crude product, which was purified by preparative high-performance liquid chromatography over Column: Phenomenex Gemini-NX 80*40 mm*3 um to give the title compound 2-(4,4-difluoropiperidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (52 mg, 61%) as an off-white solid. LCMS (ESI): mass calcd. for C₂₃H₂₇F₂N₁₁O, 511.2; m/z found, 512.2 [M+H]⁺. ¹H NMR (400 MHz, METHANOL-d₄) δ 8.85 (s, 1H), 8.68 (d, J=2.1 Hz, 1H), 8.38 (d, J=2.1 Hz, 1H), 8.12 (s, 1H), 7.67 (s, 1H), 3.93 (s, 3H), 3.86 (s, 3H), 3.29 (s, 2H), 2.74 (br t, J=5.4 Hz, 4H), 2.57 (s, 3H), 2.16-2.03 (m, 4H).

### Example 73. 2-(trans-2,6-dimethylmorpholino)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

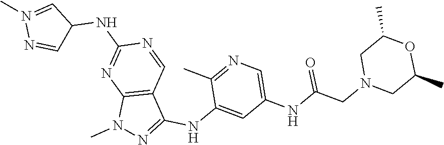

To a solution of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (55 mg, 0.13 mmol) and potassium carbonate (53 mg, 0.39 mmol), sodium iodide (21 mg, 0.14 mmol) in N,N-dimethylformamide (3 mL) was added trans-2,6-dimethylmorpholine (45 mg, 0.39 mmol) at room-temperature. The resulting mixture was stirred at 50° C. for 1.5 h before cooling to room-temperature. The reaction mixture was filtered and the filtrate was concentrated under vacuum to give crude product, which was purified by preparative high-performance liquid chromatography over Column: Phenomenex Gemini-NX 80*40 mm*3 um to give the title compound 2-(trans-2,6-dimethylmorpholino)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (38.8 mg, 57%) as a white solid. LCMS (ESI): mass calcd. for C₂₄H₃N₁₁O₂, 505.5; m/z found, 506.3 [M+H]⁺. ¹H NMR (400 MHz, METHANOL-d4) δ 8.84 (s, 1H), 8.66 (d, J=2.1 Hz, 1H), 8.39 (d, J=2.1 Hz, 1H), 8.08 (s, 1H), 7.65 (s, 1H), 4.15-4.05 (m, 2H), 3.91 (s, 3H), 3.83 (s, 3H), 3.22 (d, J=15.6 Hz, 1H), 3.04 (d, J=15.7 Hz, 1H), 2.62 (dd, J=3.0, 11.0 Hz, 2H), 2.55 (s, 3H), 2.32 (dd, J=5.7, 11.0 Hz, 2H), 1.31 (s, 3H), 1.30 (s, 3H).

### Example 74. N-(6-methyl-5-((1-methyl-6-((1-methyl-1H-pyrazol-4-yl)amino)-1H-pyrazolo[3,4-d]pyrimidin-3-yl)amino)pyridin-3-yl)-2-(5-azaspiro[2.4]heptan-5-yl)acetamide

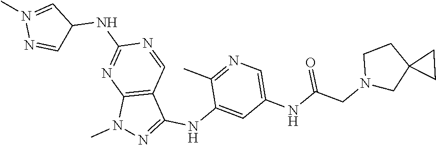

To a solution of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (55 mg, 0.13 mmol) and potassium carbonate (53 mg, 0.39 mmol), sodium iodide (21 mg, 0.14 mmol) in N,N-dimethylformamide (3 mL) was added 5-azaspiro[2.4]heptane hydrochloride (52 mg, 0.39 mmol) at room-temperature. The resulting mixture was stirred at 50° C. for 1.5 h before cooling to room-temperature. The reaction mixture was filtered and the filtrate was concentrated under vacuum to give crude product, which was purified by preparative high-performance liquid chromatography over Column: Phenomenex Gemini-NX 80*40 mm*3 um to give the title compound N-(6-methyl-5-((1-methyl-6-((1-methyl-1H-pyrazol-4-yl)amino)-1H-pyrazolo[3,4-d]pyrimidin-3-yl)amino)pyridin-3-yl)-2-(5-azaspiro[2.4]heptan-5-yl)acetamide (39.3 mg, 58%) as a white solid. LCMS (ESI): mass calcd. for C₂₄H₂₉N₁₁O, 487.5; m/z found, 488.3 [M+H]⁺. ¹H NMR (400 MHz, METHANOL-d₄) δ 8.82 (s, 1H), 8.64 (d, J=2.1 Hz, 1H), 8.34 (d, J=2.1 Hz, 1H), 8.07 (s, 1H), 7.64 (s, 1H), 3.89 (s, 3H), 3.82 (s, 3H), 3.36 (s, 2H), 2.90 (t, J=6.9 Hz, 2H), 2.67 (s, 2H), 2.52 (s, 3H), 1.87 (t, J=6.9 Hz, 2H), 0.63-0.59 (m, 2H), 0.58-0.54 (m, 2H).

### Example 75. 2-(3,3-difluoropyrrolidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

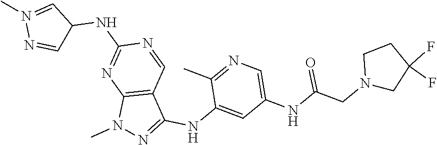

To a solution of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (40 mg, 0.09 mmol) and potassium carbonate (39 mg, 0.28 mmol), sodium iodide (19.3 mg, 0.1 mmol) in N,N-dimethylformamide (3 mL) was added 3,3-difluoropyrrolidine hydrochloride (40 mg, 0.28 mmol) at room-temperature. The resulting mixture was stirred at 50° C. for 2 h before cooling to room-temperature. The reaction mixture was filtered and the filtrate was concentrated under vacuum to give crude product, which was purified by preparative high-performance liquid chromatography over Column: Phenomenex Gemini-NX C18 75*30 mm*3 um to give the title compound 2-(3,3-difluoropyrrolidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide as a formate salt (19.3 mg, 36%) as a white solid. LCMS (ESI): mass calcd. for C₂₂H₂₅F₂N₁₁O, 497.5; m/z found, 498.3 [M+H]⁺. ¹H NMR (400 MHz, METHANOL-d₄) δ 8.81 (s, 1H), 8.70 (d, J=2.0 Hz, 1H), 8.34 (d, J=2.0 Hz, 1H), 8.13 (br s, 1H), 8.04 (s, 1H), 7.63 (s, 1H), 3.88 (s, 3H), 3.81 (s, 3H), 3.39 (s, 2H), 3.10 (t, J=13.3 Hz, 2H), 2.94 (t, J=7.0 Hz, 2H), 2.53 (s, 3H), 2.34 (tt, J=7.1, 14.6 Hz, 2H).

### Example 76. 2-(3,3-difluoroazetidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

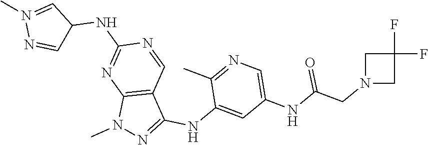

To a solution of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (40 mg, 0.09 mmol) and potassium carbonate (39 mg, 0.28 mmol), sodium iodide (19.3 mg, 0.1 mmol) in N,N-dimethylformamide (3 mL) was added 3,3-difluoroazetidine hydrochloride (36 mg, 0.28 mmol) at room-temperature. The resulting mixture was stirred at 50° C. for 2 h before cooling to room-temperature. The reaction mixture was filtered and the filtrate was concentrated under vacuum to give crude product, which was purified by preparative high-performance liquid chromatography over Column: Phenomenex Gemini-NX C18 75*30 mm*3 um to give the title compound 2-(3,3-difluoroazetidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide as formate salt (26.9 mg, 50%) as a white solid. LCMS (ESI): mass calcd. for C₂₁H₂₃F₂N₁₁O, 483.4; m/z found, 484.2 [M+H]⁺. ¹H NMR (400 MHz, METHANOL-d₄) δ 8.85 (s, 1H), 8.67 (d, J=2.0 Hz, 1H), 8.40-8.35 (m, 1H), 8.16 (br s, 1H), 8.10 (s, 1H), 7.66 (s, 1H), 3.94-3.82 (m, 10H), 3.53 (s, 2H), 2.56 (s, 3H).

### Example 77. 2-(3,3-dimethylazetidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

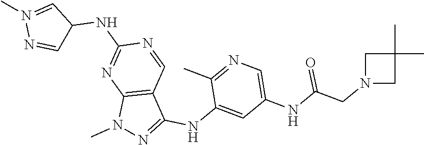

To a solution of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (44 mg, 0.1 mmol) and potassium carbonate (43 mg, 0.31 mmol), sodium iodide (17 mg, 0.1 mmol) in N,N-dimethylformamide (3 mL) was added 3,3-dimethylazetidine hydrochloride (38 mg, 0.31 mmol) at room-temperature. The resulting mixture was stirred at 50° C. for 1.5 h before cooling to room-temperature. The reaction mixture was filtered and the filtrate was concentrated under vacuum to give crude product, which was purified by preparative high-performance liquid chromatography over Column: Phenomenex Gemini-NX 80*40 mm*3 um to give the title compound 2-(3,3-dimethylazetidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (22.8 mg, 45%) as a white solid. LCMS (ESI): mass calcd. for C₂₃H₂₉N₁₁O, 475.5; m/z found, 476.3 [M+H]⁺. ¹H NMR (400 MHz, METHANOL-d₄) δ 8.83 (s, 1H), 8.64 (d, J=2.1 Hz, 1H), 8.32 (d, J=2.3 Hz, 1H), 8.08 (s, 1H), 7.65 (s, 1H), 3.90 (s, 3H), 3.83 (s, 3H), 3.34 (s, 2H), 3.19 (s, 4H), 2.53 (s, 3H), 1.27 (s, 6H).

### Example 78. 2-(cis-2,6-dimethylmorpholino)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

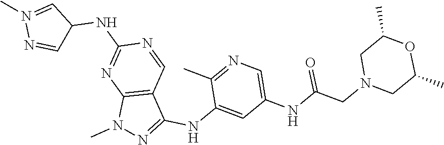

To a solution of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (44 mg, 0.1 mmol) and potassium carbonate (43 mg, 0.31 mmol), sodium iodide (17 mg, 0.1 mmol) in N,N-dimethylformamide (3 mL) was added cis-2,6-dimethylmorpholine (36 mg, 0.31 mmol) at room-temperature. The resulting mixture was stirred at 50° C. for 1.5 h before cooling to room-temperature. The reaction mixture was filtered and the filtrate was concentrated under vacuum to give crude product, which was purified by preparative high-performance liquid chromatography over Column: Phenomenex Gemini-NX 80*40 mm*3 um to give the title compound 2-(cis-2,6-dimethylmorpholino)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (33.6 mg, 63%) as a white solid. LCMS (ESI): mass calcd. for C₂₄H₃₁N₁O₂, 505.5; m/z found, 506.3 [M+H]⁺. ¹H NMR (400 MHz, METHANOL-d₄) δ 8.83 (s, 1H), 8.65 (d, J=2.1 Hz, 1H), 8.35 (d, J=2.1 Hz, 1H), 8.09 (s, 1H), 7.65 (s, 1H), 3.91 (s, 3H), 3.84 (s, 3H), 3.82-3.75 (m, 2H), 3.18 (s, 2H), 2.83 (br d, J=10.5 Hz, 2H), 2.55 (s, 3H), 1.97-1.87 (m, 2H), 1.15 (s, 3H), 1.13 (s, 3H).

### Example 79. 2-(cyclopentyl(2-hydroxyethyl)amino)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

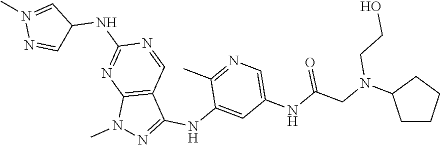

To a solution of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (60 mg, 0.14 mmol) and potassium carbonate (58 mg, 0.42 mmol), sodium iodide (23 mg, 0.15 mmol) in N,N-dimethylformamide (3 mL) was added 2-(cyclopentylamino)ethanol (55 mg, 0.42 mmol) at room-temperature. The resulting mixture was stirred at 50° C. for 2 h before cooling to room-temperature. The reaction mixture was filtered and the filtrate was concentrated under vacuum to give crude product, which was purified by preparative high-performance liquid chromatography over Column: Phenomenex Gemini-NX 80*40 mm*3 um to give the title compound 2-(cyclopentyl(2-hydroxyethyl)amino)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (46 mg, 59%) as a white solid. LCMS (ESI): mass calcd. for C₂₅H₃₃N₁₁O₂, 519.6; m/z found, 520.4 [M+H]⁺. ¹H NMR (400 MHz, METHANOL-d₄) δ 8.85 (s, 1H), 8.72 (d, J=2.1 Hz, 1H), 8.35 (d, J=2.1 Hz, 1H), 8.11 (s, 1H), 7.67 (s, 1H), 3.93 (s, 3H), 3.86 (s, 3H), 3.68 (t, J=5.2 Hz, 2H), 3.32 (br s, 2H), 3.30-3.26 (m, 1H), 2.81 (t, J=5.2 Hz, 2H), 2.56 (s, 3H), 1.88 (br d, J=6.8 Hz, 2H), 1.77-1.66 (m, 2H), 1.66-1.55 (m, 2H), 1.52-1.42 (m, 2H).

### Example 80. 2-(cyclopentyl(3-hydroxypropyl)amino)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

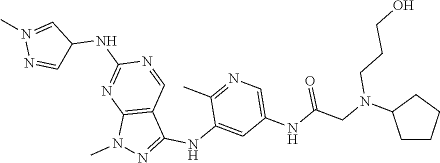

To a mixture of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (70 mg, 0.16 mmol), 3-(cyclopentylamino)propan-1-ol (70.5 mg, 0.45 mmol) and NaI (27 mg, 0.18 mmol) in DMF (4 mL) was added K₂CO₃ (68 mg, 0.49 mmol) at room temperature. The mixture was stirred at 50° C. for 2 hours. The reaction mixture was filtered and the filtrate was concentrated under vacuum to give crude product, which was purified by preparative high-performance liquid chromatography over column: Phenomenex Gemini-NX 80*40 mm*3 um to give the title compound 2-(cyclopentyl(3-hydroxypropyl)amino)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (37 mg, 41%) as a pale brown solid.

LCMS (ESI): mass calcd. for C₂₆H₃₅N₁₁O₂, 533.629; m/z found, 534.3 [M+H]⁺. ¹H NMR (400 MHz, METHANOL-d₄) δ 8.85 (s, 1H), 8.69 (d, J=2.1 Hz, 1H), 8.35 (d, J=2.1 Hz, 1H), 8.11 (s, 1H), 7.67 (s, 1H), 3.92 (s, 3H), 3.86 (s, 3H), 3.67 (t, J=6.0 Hz, 2H), 3.30 (s, 2H), 3.28-3.20 (m, 1H), 2.79 (t, J=7.2 Hz, 2H), 2.56 (s, 3H), 1.88 (br d, J=6.4 Hz, 2H), 1.81-1.68 (m, 4H), 1.66-1.56 (m, 2H), 1.55-1.44 (m, 2H).

### Example 81. (S)—N-(2-(3-ethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide

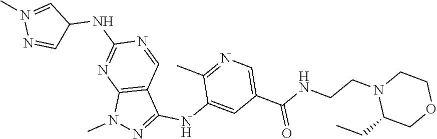

### Step a: (S)-2-(3-ethylmorpholino)acetonitrile

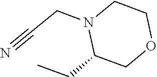

To a solution of (S)-3-ethylmorpholine (0.5 g, 4.3 mmol) and potassium carbonate (1.2 g, 8.7 mmol) in acetonitrile (10 mL) was added 2-bromoacetonitrile (0.63 g, 5.2 mmol) at room temperature. The resulting mixture was stirred at 50° C. for 12 h before cooling to room-temperature. The reaction mixture was filtered. The residue was washed with ethyl acetate (50 mL×3) and concentrated under reduced pressure to give crude product, which was purified by column chromatography over silica gel (eluent: petroleum ether:ethyl acetate=3:1) to give the title compound (S)-2-(3-ethylmorpholino)acetonitrile (600 mg, 90%) as a pale brown oil. LCMS (ESI): mass calcd. for C₈H₁₄N₂O, 154.1; m/z found, 155.1 [M+H]⁺. ¹H NMR (400 MHz, CDCl₃) δ 3.83-3.73 (m, 3H), 3.57 (dt, J=2.5, 11.3 Hz, 1H), 3.37 (d, J=17.4 Hz, 1H), 3.25 (t, J=10.7 Hz, 1H), 2.71 (dt, J=3.2, 11.2 Hz, 1H), 2.55 (br d, J=11.3 Hz, 1H), 2.50-2.41 (m, 1H), 1.58-1.47 (m, 1H), 1.31 (quind, J=7.5, 14.8 Hz, 1H), 0.84 (t, J=7.6 Hz, 3H).

### Step b: (S)-2-(3-ethylmorpholino)ethanamine

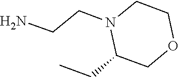

To a solution of (S)-2-(3-ethylmorpholino)acetonitrile (300 mg, 1.94 mmol) in THF (10 mL) was added lithium aluminium hydride (111 mg, 2.92 mmol) by portions at 0° C. (ice/water), and the resulting mixture was stirred at 25° C. for 1 h. After cooled to 0° C., the reaction mixture was quenched with water (0.25 mL) and filtered. The filtrate was concentrated to dryness under reduced pressure to afford the crude product (S)-2-(3-ethylmorpholino)ethanamine (260 mg, 84%) as a yellow oil. LCMS (ESI): mass calcd. for C₈H₁₈N₂O 2, 158.2; m/z found, 159.2[M+H]⁺. ¹H NMR (400 MHz, CDCl₃) δ 3.72-3.64 (m, 2H), 3.60-3.52 (m, 1H), 3.34 (dd, J=7.9, 11.3 Hz, 1H), 2.79-2.61 (m, 4H), 2.27-2.13 (m, 3H), 1.56-1.46 (m, 1H), 1.41-1.36 (m, 1H), 0.81 (t, J=7.6 Hz, 3H).

### Step c: (S)—N-(2-(3-ethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide

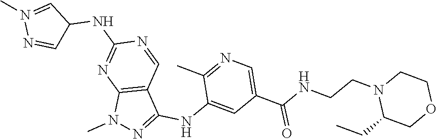

To a solution of 6-methyl-5-((1-methyl-6-((1-methyl-1H-pyrazol-4-yl)amino)-1H-pyrazolo[3,4-d]pyrimidin-3-yl)amino)nicotinic acid (160 mg, 0.41 mmol), HATU (283 mg, 0.74 mmol) and N,N-diisopropylethylamine (0.2 mL, 1.24 mmol) in N,N-dimethylformamide (7 mL) was added (S)-2-(3-ethylmorpholino)ethanamine (118 mg, 0.74 mmol). The mixture stirred at room temperature for 1 h and then concentrated under vacuum to give the crude product, which was purified by preparative high-performance liquid chromatography over column: Phenomenex Gemini-NX C18 75*30 mm*3 um to give the title compound (S)—N-(2-(3-ethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide (FA salt, 67 mg, 28%) as a pale brown solid. LCMS (ESI): mass calcd. for C₂₅H₃₃N₁₁O, 519.6 m/z found, 520.2 [M+H]⁺. ¹H NMR (400 MHz, METHANOL-d₄) δ 8.89 (s, 1H), 8.78 (d, J=1.9 Hz, 1H), 8.48 (d, J=1.8 Hz, 1H), 8.23 (br s, 1H), 8.11 (s, 1H), 7.68 (s, 1H), 3.93 (s, 3H), 3.90-3.83 (m, 5H), 3.77-3.70 (m, 1H), 3.67-3.58 (m, 2H), 3.52 (dd, J=7.8, 12.1 Hz, 1H), 3.27-3.14 (m, 2H), 2.87-2.70 (m, 3H), 2.68 (s, 3H), 1.79 (ddd, J=3.1, 7.7, 13.9 Hz, 1H), 1.63-1.49 (m, 1H), 0.96 (t, J=7.5 Hz, 3H).

### Example 82. (R)-6-methyl-5-((1-methyl-6-((1-methyl-1H-pyrazol-4-yl)amino)-1H-pyrazolo[3,4-d]pyrimidin-3-yl)amino)-N-(2-(2-methylpiperidin-1-yl)ethyl)nicotinamide

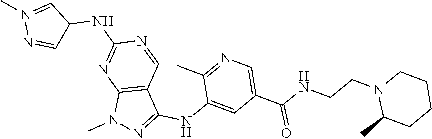

### Step a: (R)-2-(2-methylpiperidin-1-yl)acetonitrile

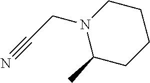

To a solution of (R)-2-methylpiperidine (0.5 g, 5.0 mmol) and potassium carbonate (1.5 g, 11 mmol) in TBME (8 mL) was added 2-bromoacetonitrile (0.665 g, 5.5 mmol) at room temperature. The resulting mixture was stirred at room temperature for 15 h. The resulting mixture was quenched with water (20 mL) and extracted with ethyl acetate (50 mL×3). The combined organic phases were dried over anhydrous Na₂SO₄ and filtered. The filtrate was concentrated under reduced pressure to give crude product, which was purified by column chromatography over silica gel (eluent: petroleum ether:ethyl acetate from 100:0 to 90:10) to give the title compound (R)-2-(2-methylpiperidin-1-yl)acetonitrile (500 mg, 72%) as a pale yellow oil. LCMS (ESI): mass calcd. for C₈H₁₄N₂, 138.1; m/z found, 139.1 [M+H]⁺. ¹H NMR (400 MHz, CDCl₃) δ 3.81 (d, J=17.4 Hz, 1H), 3.33 (d, J=17.4 Hz, 1H), 2.68 (br d, J=11.1 Hz, 1H), 2.40 (dt, J=2.7, 11.6 Hz, 1H), 2.33-2.23 (m, 1H), 1.71-1.58 (m, 3H), 1.56-1.43 (m, 1H), 1.33-1.14 (m, 2H), 1.03 (d, J=6.2 Hz, 3H).

### Step b: (R)-2-(2-methylpiperidin-1-yl)ethanamine

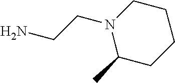

To a solution of (R)-2-(2-methylpiperidin-1-yl)acetonitrile (500 mg, 3.6 mmol) in THF (30 mL) was added lithium aluminium hydride (206 mg, 5.4 mmol) by portions at 0° C. (ice/water). The resultant mixture was stirred at room temperature for 1 h before quenched with water (0.5 mL) at 0° C. The reaction mixture was filtered. And the filtrate was concentrated to dryness under reduced pressure to afford the crude product (R)-2-(2-methylpiperidin-1-yl)ethanamine (390 mg, 76%) as a yellow oil. LCMS (ESI): mass calcd. for C₈H₁₈N₂, 142.1; m/z found, 143.1 [M+H]⁺. ¹H NMR (400 MHz, 400 MHz, CDCl₃) δ 2.83-2.61 (m, 4H), 2.28-2.15 (m, 2H), 2.09-2.01 (m, 1H), 1.63-1.41 (m, 4H), 1.26-1.19 (m, 2H), 0.99 (d, J=6.3 Hz, 3H).

### Step c: (R)-6-methyl-5-((1-methyl-6-((1-methyl-1H-pyrazol-4-yl)amino)-1H-pyrazolo[3,4-d]pyrimidin-3-yl)amino)-N-(2-(2-methylpiperidin-1-yl)ethyl)nicotinamide

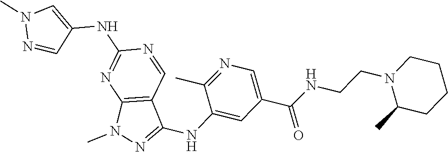 [SMILES: Cc1ncc(C(=O)NCCN2CCCC[C@H]2C)cc1Nc1nn(C)c2nc(Nc3cnn(C)c3)ncc12]

To a solution of 6-methyl-5-((1-methyl-6-(pyrimidin-5-ylamino)-1H-pyrazolo[3,4-d]pyrimidin-3-yl)amino) nicotinic acid (60 mg, 0.16 mmol), HATU (106 mg, 0.28 mmol) and N,N-diisopropylethylamine (60 mg, 0.47 mmol) in DMF (3 mL) was added (R)-2-(2-methylpiperidin-1-yl)ethanamine (35 mg, 0.25 mmol). The resulting mixture was stirred at room temperature for 1 h and then concentrated under vacuum to give the crude product, which was purified by preparative high-performance liquid chromatography over Column: Phenomenex Gemini-NX C18 75*30 mm*3 um to give the title compound (R)-6-methyl-5-((1-methyl-6-((1-methyl-1H-pyrazol-4-yl)amino)-1H-pyrazolo[3,4-d]pyrimidin-3-yl)amino)-N-(2-(2-methylpiperidin-1-yl)ethyl)nicotinamide (42 mg, FA salt, 48%) as a brown solid. LCMS (ESI): mass calcd. for C₂₅H₃₃N₁₁O, 503.3; m/z found, 504.3 [M+H]⁺. ¹H NMR (400 MHz, METHANOL-d₄) δ 8.90 (s, 1H), 8.82 (d, J=1.9 Hz, 1H), 8.50 (d, J=1.9 Hz, 1H), 8.38 (s, 1H), 8.11 (s, 1H), 7.69 (s, 1H), 3.93 (s, 3H), 3.88 (s, 3H), 3.82-3.75 (m, 1H), 3.82-3.75 (m, 1H), 3.61 (br s, 1H), 3.50 (br s, 2H), 3.31-3.26 (m, 1H), 3.16 (br d, J=10.5 Hz, 1H), 2.69 (s, 3H), 2.04-1.90 (m, 2H), 1.86 (br d, J=10.7 Hz, 2H), 1.76-1.57 (m, 2H), 1.43 (d, J=6.6 Hz, 3H).

### Example 83. N-(2-(6-azaspiro[3.4]octan-6-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

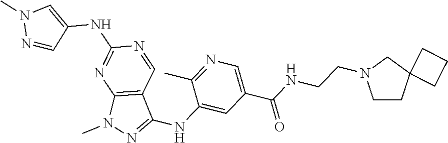 [SMILES: Cc1ncc(C(=O)NCCN2CCC3(CCC3)C2)cc1Nc1nn(C)c2nc(Nc3cnn(C)c3)ncc12]

To a solution of 6-methyl-5-((1-methyl-6-(pyrimidin-5-ylamino)-1H-pyrazolo[3,4-d]pyrimidin-3-yl)amino) nicotinic acid (100 mg, 0.24 mmol), HATU (167 mg, 0.44 mmol) and N,N-diisopropylethylamine (95 mg, 0.73 mmol) in DMF (6 mL) was added 2-(6-azaspiro[3.4]octan-6-yl)ethanamine (60 mg, 0.39 mmol). The resulting mixture was stirred at room temperature for 1 h and then concentrated under vacuum to give the crude product, which was purified by preparative high-performance liquid chromatography over Column: Welch Xtimate C18 150*30 mm*5 um to give the title compound N-(2-(6-azaspiro[3.4]octan-6-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (38 mg, 30%) as a white solid. LCMS (ESI): mass calcd. for C₂₆H₃₃N₁₁O, 515.3; m/z found, 516.3 [M+H]⁺. ¹H NMR (400 MHz, METHANOL-d₄) δ 8.87 (s, 1H), 8.77 (d, J=1.9 Hz, 1H), 8.48 (d, J=1.9 Hz, 1H), 8.11 (s, 1H), 7.67 (s, 1H), 3.93 (s, 3H), 3.87 (s, 3H), 3.56 (t, J=6.9 Hz, 2H), 2.78-2.69 (m, 6H), 2.67 (s, 3H), 2.13-1.94 (m, 6H), 1.92-1.80 (m, 2H).

### Example 84. N-(2-(cyclobutyl(2-methoxyethyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide

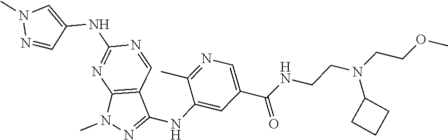

### Step a: N-(2-methoxyethyl)cyclobutanamine

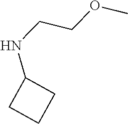

To a solution of 2-methoxyethanamine (1.5 g, 20 mmol), cyclobutanone (1.54 g, 22 mmol) and AcOH (1.3 mL, 22 mmol) in MeOH (21 mL) was added NaBH₃CN (2.5 g, 40 mmol) portion wise at 0° C. The mixture was stirred at room temperature for 12 h. The reaction was quenched with saturated aq NaHCO₃ (20 mL). The mixture was extracted with DCM (80 mL*5). The organic layer was washed with brine, dried over anhydrous Na₂SO₄, filtered and concentrated in vacuo to give crude of N-(2-methoxyethyl)cyclobutanamine (380 mg, 15%) as pale brown oil. ¹H NMR (400 MHz, CDCl₃) δ 3.47-3.41 (m, 2H), 3.30 (s, 3H), 3.29-3.22 (m, 1H), 2.75 (br s, 1H), 2.71-2.68 (m, 2H), 2.22-2.12 (m, 2H), 1.81-1.57 (m, 4H).

### Step b: 2-(cyclobutyl(2-methoxyethyl)amino)acetonitrile

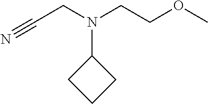

To a solution of N-(2-methoxyethyl)cyclobutanamine (430 mg, 3.3 mmol) and potassium carbonate (1.4 g, 10 mmol) in acetonitrile (10 mL) was added 2-bromoacetonitrile (439 mg, 3.7 mmol) at room temperature. The resulting mixture was stirred at 50° C. for 12 h before cooling to room-temperature. The reaction mixture was filtered. The filter cake was washed with ethyl acetate (50 mL×3). The combined filtrate was concentrated under reduced pressure to give crude product, which was purified by column chromatography over silica gel (eluent: petroleum ether/ethyl acetate=3/1) to give the title compound 2-(cyclobutyl-(2-methoxyethyl)amino)acetonitrile (440 mg, 79%) as a pale yellow oil. ¹H NMR (400 MHz, CDCl₃) δ 3.66 (s, 2H), 3.51 (t, J=5.2 Hz, 2H), 3.38 (s, 3H), 3.22-3.09 (m, 1H), 2.64 (t, J=5.2 Hz, 2H), 2.16-2.05 (m, 2H), 1.94-1.82 (m, 2H), 1.78-1.66 (m, 2H).

### Step c: N¹-cyclobutyl-N¹-(2-methoxyethyl)ethane-1,2-diamine

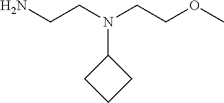

To a solution of 2-(cyclobutyl(2-methoxyethyl)amino)acetonitrile (390 mg, 2.3 mmol) in THF (10 mL) was added lithium aluminium hydride (132 mg, 3.5 mmol) by portions at 0° C. (ice/water), and the resulting mixture was stirred at 25° C. for 1 h. After cooled to 0° C., the reaction mixture was quenched with water (0.5 mL) and filtered. The filtrate was then concentrated to dryness under reduced pressure to afford the crude product N¹-cyclobutyl-N¹-(2-methoxyethyl)ethane-1,2-diamine (380 mg, 95%) as a yellow oil. ¹H NMR (400 MHz, CDCl₃) δ 3.35 (t, J=6.3 Hz, 2H), 3.27 (s, 3H), 3.06 (quin, J=8.0 Hz, 1H), 2.65 (t, J=6.4 Hz, 2H), 2.54 (t, J=6.3 Hz, 2H), 2.44-2.38 (m, 2H), 2.00-1.90 (m, 2H), 1.85-1.74 (m, 2H), 1.67-1.45 (m, 2H).

### Step d: N-(2-(cyclobutyl(2-methoxyethyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide

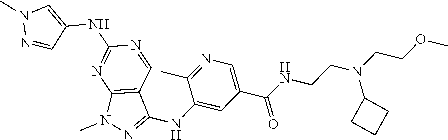

To a solution of 6-methyl-5-((1-methyl-6-(pyrimidin-5-ylamino)-1H-pyrazolo[3,4-d]pyrimidin-3-yl)amino) nicotinic acid (100 mg, 0.24 mmol), HATU (167 mg, 0.44 mmol) and N,N-diisopropylethylamine (95 mg, 0.73 mmol) in DMF (6 mL) was added Ni-cyclobutyl-N¹-(2-methoxyethyl)ethane-1,2-diamine (67 mg, 0.39 mmol). The resulting mixture was stirred at room temperature for 1 h and then concentrated under vacuum to give the crude product, which was purified by preparative high-performance liquid chromatography over Column: Welch Xtimate C18 150*30 mm*5 um to give the title compound N-(2-(cyclobutyl(2-methoxyethyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide (40 mg, 30%) as a white solid. LCMS (ESI): mass calcd. for C₂₆H₃₅N₁₁O₂, 533.3; m/z found, 534.4 [M+H]⁺. ¹H NMR (400 MHz, METHANOL-d₄) δ 9.11 (s, 1H), 8.99 (d, J=1.9 Hz, 1H), 8.70 (d, J=1.9 Hz, 1H), 8.35 (s, 1H), 7.90 (s, 1H), 4.16 (s, 3H), 4.11 (s, 3H), 3.76-3.67 (m, 4H), 3.55-3.53 (m, 3H), 3.51-3.46 (m, 1H), 2.99-2.92 (m, 4H), 2.90 (s, 3H), 2.35-2.27 (m, 2H), 2.19-2.09 (m, 2H), 1.94-1.85 (m, 2H).

### Example 85. N-(2-(2-azabicyclo[2.2.2]octan-2-yl)ethyl)-6-methyl-5-((1-methyl-6-((1-methyl-1H-pyrazol-4-yl)amino)-1H-pyrazolo [3,4-d]pyrimidin-3-yl)amino)nicotinamide

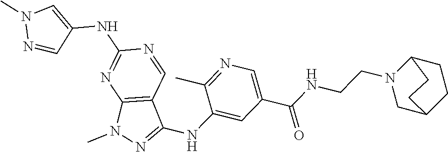 [SMILES: Cc1ncc(C(=O)NCCN2CC3CCC2CC3)cc1Nc1nn(C)c2nc(Nc3cnn(C)c3)ncc12]

### Step a: N-(2-(2-azabicyclo[2.2.2]octan-2-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

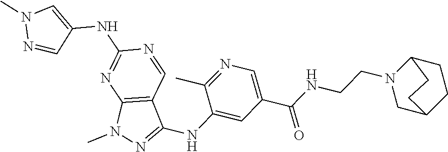 [SMILES: Cc1ncc(C(=O)NCCN2CC3CCC2CC3)cc1Nc1nn(C)c2nc(Nc3cnn(C)c3)ncc12]

To a solution of 6-methyl-5-((1-methyl-6-(pyrimidin-5-ylamino)-1H-pyrazolo[3,4-d]pyrimidin-3-yl)amino) nicotinic acid (100 mg, 0.24 mmol), HATU (167 mg, 0.44 mmol) and N,N-diisopropylethylamine (95 mg, 0.73 mmol) in DMF (6 mL) was added 2-(2-azabicyclo[2.2.2]octan-2-yl)ethanamine (60 mg, 0.39 mmol). The resulting mixture was stirred at room temperature for 1 h and then concentrated under vacuum to give the crude product, which was purified by preparative high-performance liquid chromatography over Column: Welch Xtimate C18 150*30 mm*5 um to give the title compound N-(2-(2-azabicyclo[2.2.2]octan-2-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (FA salt, 40 mg, 27%) as a pale brown solid. LCMS (ESI): mass calcd. for C₂₆H₃₃N₁₁O, 515.3; m/z found, 516.3 [M+H]⁺. ¹H NMR (400 MHz, METHANOL-d₄) δ 8.90 (s, 1H), 8.82 (d, J=1.7 Hz, 1H), 8.51 (br d, J=1.8 Hz, 2H), 8.11 (s, 1H), 7.69 (s, 1H), 3.93 (s, 3H), 3.88 (s, 3H), 3.75 (br t, J=6.1 Hz, 2H), 3.57 (br s, 1H), 3.44 (br t, J=6.0 Hz, 4H), 2.69 (s, 3H), 2.23-2.11 (m, 2H), 2.01 (br s, 1H), 1.92-1.76 (m, 6H).

### Example 86. N-(2-(3,3-dimethylpyrrolidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

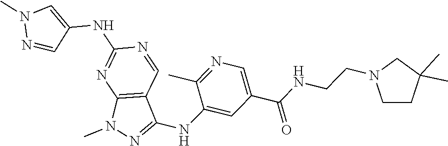

### Step a: 2-(3,3-dimethylpyrrolidin-1-yl)acetonitrile

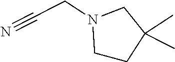

To a solution of 3,3-dimethylpyrrolidine hydrochloride (0.5 g, 3.7 mmol) and potassium carbonate (1.6 g, 12 mmol) in TBME (8 mL) was added 2-bromoacetonitrile (0.49 g, 4.1 mmol) at room temperature. The resulting mixture was stirred at room temperature for 15 h. The reaction mixture was filtered. The filter cake was washed with ethyl acetate (50 mL×3). The combined filtrate was concentrated under reduced pressure to give crude product, which was purified by column chromatography over silica gel (eluent: petroleum ether:ethyl acetate=3:1) to give the title compound title compound 2-(3,3-dimethylpyrrolidin-1-yl)acetonitrile (240 mg, 42%) as a pale yellow oil. ¹H NMR (400 MHz, CDCl₃) δ 3.56 (s, 2H), 2.70 (t, J=7.2 Hz, 2H), 2.40 (s, 2H), 1.58 (t, J=7.1 Hz, 2H), 1.04 (s, 6H).

### Step b: 2-(3,3-dimethylpyrrolidin-1-yl)ethanamine

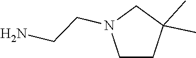

To a solution of 2-(3,3-dimethylpyrrolidin-1-yl)acetonitrile (240 mg, 1.7 mmol) in THF (15 mL) was added lithium aluminium hydride (99 mg, 2.6 mmol) by portions at 0° C. (ice/water), and the resulting mixture was stirred at 25° C. for 1 h. After cooled to 0° C., the reaction mixture was quenched with water (0.2 mL) and filtered. The filtrate was then concentrated to dryness under reduced pressure to afford the crude product 2-(3,3-dimethylpyrrolidin-1-yl)ethanamine (170 mg, 69%) as a yellow oil. ¹H NMR (400 MHz, CDCl₃) δ 2.75 (br t, J=6.1 Hz, 2H), 2.61 (br s, 2H), 2.53-2.43 (m, 2H), 2.32 (br s, 2H), 1.54 (br t, J=7.0 Hz, 2H), 1.02 (s, 6H).

### Step c: N-(2-(3,3-dimethylpyrrolidin-1-yl)ethyl)-6-methyl-5-((1-methyl-6-((1-methyl-1H-pyrazol-4-yl)amino)-1H-pyrazolo[3,4-d]pyrimidin-3-_yl)amino)nicotinamide

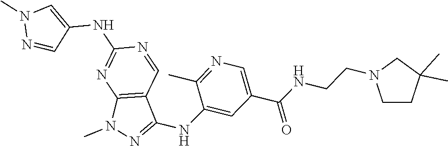

To a solution of 6-methyl-5-((1-methyl-6-((1-methyl-1H-pyrazol-4-yl)amino)-1H-pyrazolo[3,4-d]pyrimidin-3-yl)amino)nicotinic acid (120 mg, 0.29 mmol), HATU (201 mg, 0.53 mmol) and N,N-diisopropylethylamine (0.15 mL, 0.88 mmol) in N,N-dimethylformamide (8 mL) was added 2-(3,3-dimethylpyrrolidin-1-yl)ethanamine (37 mg, 0.47 mmol). The mixture stirred at room temperature for 1 h and then concentrated under vacuum to give the crude product, which was purified by preparative high-performance liquid chromatography over column: Phenomenex Gemini-NX C18 75*30 mm*3 um to give the title compound N-(2-(3,3-dimethylpyrrolidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (38 mg, 24%) as a pale brown solid. LCMS (ESI): mass calcd. for C₂₅H₃₃N₁₁O, 503.3 m/z found, 504.3 [M+H]⁺. ¹H NMR (400 MHz, METHANOL-d₄) δ 8.88 (s, 1H), 8.77 (d, J=1.8 Hz, 1H), 8.48 (d, J=1.9 Hz, 1H), 8.12 (s, 1H), 7.67 (s, 1H), 3.93 (s, 3H), 3.88 (s, 3H), 3.55 (t, J=6.9 Hz, 2H), 2.76 (td, J=6.9, 13.9 Hz, 4H), 2.67 (s, 3H), 2.51 (s, 2H), 1.66 (t, J=7.0 Hz, 2H), 1.13 (s, 6H).

### Example 87. N-(2-(3,5-dimethylpiperidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

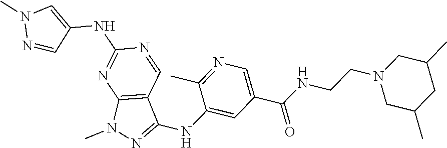

### Step a: 2-(3,5-dimethylpiperidin-1-yl)acetonitrile

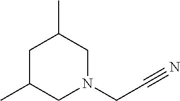

To a solution of 3,5-dimethylpiperidine (200 mg, 1.8 mmol) and potassium carbonate (538 mg, 3.9 mmol) in N,N-dimethylformamide (10 mL) was added 2-bromoacetonitrile (0.14 mL, 1.9 mmol) at room-temperature. The resulting mixture was stirred at 25° C. for 15 hours. The resulting mixture was quenched with water (10 ml) and extracted with ethyl acetate (20 ml*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=4:1) to give the title compound 2-(3,5-dimethylpiperidin-1-yl)acetonitrile (220 mg, 82%) as a yellow oil. ¹H NMR (400 MHz, CDCl₃) δ 3.51 (s, 2H), 2.69 (dt, J=9.98, 1.63 Hz, 2H), 1.79-1.95 (m, 2H), 1.58-1.76 (m, 3H), 0.87 (d, J=6.39 Hz, 6H), 0.43-0.63 (m, 1H).

### Step b: 2-(3,5-dimethylpiperidin-1-yl)ethanamine

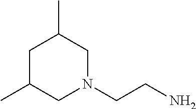

To a solution of 2-(3,5-dimethylpiperidin-1-yl)acetonitrile (170 mg, 1.1 mmol) in THF (3 mL) was added lithium aluminium hydride (51 mg, 1.3 mmol) by portions at 0° C. (ice/water). The resultant mixture was stirred at 20° C. for 1.5 h before quenched with water (0.06 mL) at 0° C. The reaction mixture was filtered. And the filtrate was concentrated to dryness under reduced pressure to afford the crude product 2-(3,5-dimethylpiperidin-1-yl)ethanamine (150 mg, 86%) as a colorless oil, which was used to the next step without further purification. ¹H NMR (400 MHz, CDCl₃) δ 2.75-2.97 (m, 4H), 2.38 (t, J=6.28 Hz, 2H), 1.85 (br s, 2H), 1.69 (br d, J=3.09 Hz, 1H), 1.37-1.53 (m, 2H), 0.84 (s, 3H), 0.82 (s, 3H), 0.41-0.58 (m, 1H).

### Step c: N-(2-(3,5-dimethylpiperidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

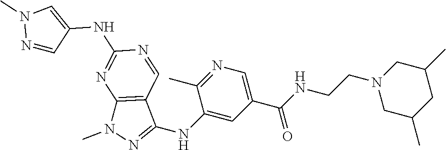

To a solution of 6-methyl-5-((1-methyl-6-((1-methyl-1H-pyrazol-4-yl)amino)-1H-pyrazolo[3,4-d]pyrimidin-3-yl)amino)nicotinic acid (170 mg, 0.44 mmol), HATU (217 mg, 0.57 mmol) and N,N-diisopropylethylamine (0.29 mL, 1.8 mmol) in N,N-dimethylformamide (3 mL) was added 2-(3,5-dimethylpiperidin-1-yl)ethanamine (69 mg, 0.44 mmol). The mixture stirred at 25° C. for 2 h and then concentrated under vacuum to give the crude product, which was purified by preparative high-performance liquid chromatography over Column: Phenomenex Gemini-NX 80*40 mm*3 um to give the title compound N-(2-(3,5-dimethylpiperidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (38 mg, 17%) as a yellow solid. LCMS (ESI): mass calcd. for C₂₆H₃₅N₁₁O, 517.6; m/z found, 518.3 [M+H]⁺. ¹H NMR (400 MHz, METHANOL-d4) δ 8.84 (s, 1H), 8.73 (d, J=1.98 Hz, 1H), 8.44 (d, J=1.76 Hz, 1H), 8.08 (s, 1H), 7.63 (s, 1H), 3.89 (s, 3H), 3.84 (s, 3H), 3.56 (t, J=6.84 Hz, 2H), 2.95 (br d, J=9.26 Hz, 2H), 2.63 (s, 3H), 2.57-2.62 (m, 2H), 1.56-1.76 (m, 5H), 0.88 (s, 3H), 0.86 (s, 3H), 0.48-0.61 (m, 1H).

### Example 88. (R)—N-(2-(2-ethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide

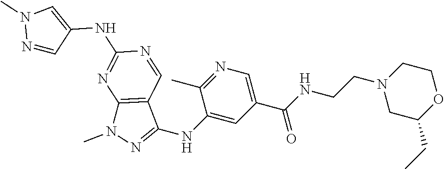 [SMILES: CC[C@@H]1CN(CCNC(=O)c2cnc(C)c(Nc3nn(C)c4nc(Nc5cnn(C)c5)ncc34)c2)CCO1]

### Step a: (R)-2-(2-ethylmorpholino)acetonitrile

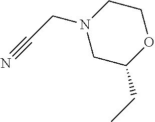

To a solution of (R)-2-ethylmorpholine (200 mg, 1.7 mmol) and potassium carbonate (528 mg, 3.8 mmol) in N,N-dimethylformamide (5 mL) was added 2-Bromoacetonitrile (0.13 mL, 1.9 mmol) at room-temperature. The resulting mixture was stirred at 25° C. for 15 hours. The resulting mixture was quenched with water (10 ml) and extracted with ethyl acetate (20 ml*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=4:1) to give the title compound (R)-2-(2-ethylmorpholino)acetonitrile (188 mg, 70%) as a yellow oil. ¹H NMR (400 MHz, CDCl₃) δ 3.58-3.96 (m, 2H), 3.50 (s, 2H), 3.34-3.43 (m, 1H), 2.55-2.71 (m, 2H), 2.09-2.54 (m, 2H), 1.37-1.59 (m, 2H), 0.93 (t, J=7.50 Hz, 3H).

### Step b: (R)-2-(2-ethylmorpholino)ethanamine

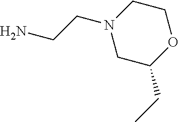

To a solution of (R)-2-(2-ethylmorpholino)acetonitrile (170 mg, 1.1 mmol) in THE (3 mL) was added lithium aluminium hydride (50 mg, 1.3 mmol) by portions at 0° C. (ice/water). The resultant mixture was stirred at 20° C. for 1.5 h before quenched with water (0.06 mL) at 0° C. The reaction mixture was filtered. And the filtrate was concentrated to dryness under reduced pressure to afford the crude product (R)-2-(2-ethylmorpholino)ethanamine (150 mg, 86%) as a colorless oil, which was used to the next step without further purification. ¹H NMR (400 MHz, CDCl₃) δ 3.80-3.92 (m, 1H), 3.44-3.79 (m, 2H), 2.79-3.42 (m, 2H), 2.36-2.77 (m, 4H), 1.75-2.19 (m, 2H), 1.31-1.59 (m, 2H), 0.87-1.05 (m, 3H).

### Step c: (R)—N-(2-(2-ethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide

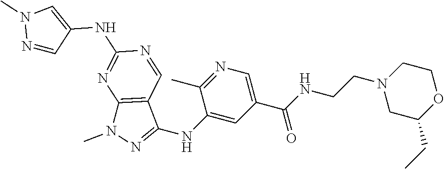 [SMILES: CC[C@@H]1CN(CCNC(=O)c2cnc(C)c(Nc3nn(C)c4nc(Nc5cnn(C)c5)ncc34)c2)CCO1]

To a solution of 6-methyl-5-((1-methyl-6-((1-methyl-1H-pyrazol-4-yl)amino)-1H-pyrazolo[3,4-d]pyrimidin-3-yl)amino)nicotinic acid (110 mg, 0.28 mmol), HATU (141 mg, 0.37 mmol) and N,N-diisopropylethylamine (0.19 mL, 1.1 mmol) in N,N-dimethylformamide (3 mL) was added (R)-2-(2-ethylmorpholino)ethanamine (45 mg, 0.28 mmol). The mixture stirred at 25° C. for 2 h and then concentrated under vacuum to give the crude product, which was purified by preparative high-performance liquid chromatography over Column: Boston Prime C18 150*30 mm*5 um to give the title compound (R)—N-(2-(2-ethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide (40 mg, 27%) as a brown solid. LCMS (ESI): mass calcd. for C₂₅H₃₃N₁₁O₂, 519.6; m/z found, 520.4 [M+H]⁺. ¹H NMR (400 MHz, METHANOL-d₄) δ 8.88 (s, 1H), 8.76 (d, J=1.76 Hz, 1H), 8.48 (d, J=1.51 Hz, 1H), 8.11 (s, 1H), 7.67 (s, 1H), 3.93 (s, 3H), 3.88 (s, 3H), 3.86 (br s, 1H), 3.53-3.69 (m, 4H), 2.82-2.94 (m, 2H), 2.67 (s, 3H), 2.63 (br t, J=6.65 Hz, 2H), 2.21 (td, J=11.48, 3.14 Hz, 1H), 1.90 (t, J=10.54 Hz, 1H), 1.42-1.55 (m, 2H), 0.95 (t, J=7.53 Hz, 3H).

### Example 89. N-(2-(9-azabicyclo[3.3.1]nonan-9-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

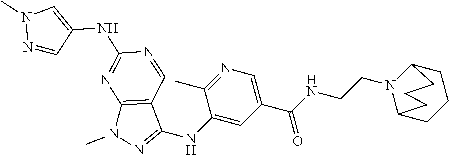 [SMILES: Cc1ncc(C(=O)NCCN2C3CCCC2CCC3)cc1Nc1nn(C)c2nc(Nc3cnn(C)c3)ncc12]

### Step a: 2-(9-azabicyclo[3.3.1]nonan-9-yl)acetonitrile

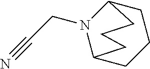 [SMILES: N#CCN1C2CCCC1CCC2]

To a solution of 9-azabicyclo[3.3.1]nonane hydrochloride (300 mg, 1.9 mmol) and potassium carbonate (564 mg, 4.1 mmol) in MeCN (10 mL) was added 2-Bromoacetonitrile (0.14 mL, 2.0 mmol) at room-temperature. The resulting mixture was stirred at 25° C. for 15 hours. The resulting mixture was quenched with water (10 ml) and extracted with ethyl acetate (20 ml*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=4:1) to give the title compound 2-(9-azabicyclo[3.3.1]nonan-9-yl)acetonitrile (280 mg, 92%) as a yellow oil. ¹H NMR (400 MHz, METHANOL-d₄) δ 3.74-3.83 (m, 2H), 2.93 (br s, 2H), 1.95-2.09 (m, 6H), 1.49-1.66 (m, 6H).

### Step b: 2-(9-azabicyclo[3.3.1]nonan-9-yl)ethanamine

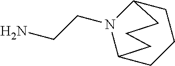 [SMILES: NCCN1C2CCCC1CCC2]

To a solution of 2-(9-azabicyclo[3.3.1]nonan-9-yl)acetonitrile (270 mg, 1.6 mmol) in THF (5 mL) was added lithium aluminium hydride (75 mg, 2.0 mmol) by portions at 0° C. (ice/water). The resultant mixture was stirred at 20° C. for 1.5 h before quenched with water (0.1 mL) at 0° C. The reaction mixture was filtered. And the filtrate was concentrated to dryness under reduced pressure to afford the crude product 2-(9-azabicyclo[3.3.1]nonan-9-yl)ethanamine (210 mg, 76%) as a colorless oil, which was used to the next step without further purification. ¹H NMR (400 MHz, METHANOL-d₄) δ 2.70-3.06 (m, 4H), 2.47-2.67 (m, 2H), 1.97-2.17 (m, 6H), 1.45-1.64 (m, 6H).

### Step c: N-(2-(9-azabicyclo[3.3.1]nonan-9-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

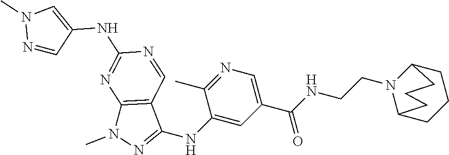 [SMILES: Cc1ncc(C(=O)NCCN2C3CCCC2CCC3)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (200 mg, 0.48 mmol), HATU (236 mg, 0.62 mmol) and N,N-diisopropylethylamine (0.32 mL, 1.9 mmol) in N,N-dimethylformamide (5 mL) was added N³-(5-amino-2-methylpyridin-3-yl)-1-methyl-N⁶-(1-methyl-1H-pyrazol-4-yl)-1H-pyrazolo[3,4-d]pyrimidine-3,6-diamine (120 mg, 0.72 mmol).

The mixture stirred at 25° C. for 2 h and then concentrated under vacuum to give the crude product, which was purified by preparative high-performance liquid chromatography over Column: Welch Xtimate C18 150*30 mm*5 um to give the title compound N-(2-(9-azabicyclo[3.3.1]nonan-9-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (44 mg, 16%) as a yellow solid. LCMS (ESI): mass calcd. for C₂₇H₃₅N₁₁O, 529.6; m/z found, 530.4 [M+H]⁺. ¹H NMR (400 MHz, DMSO-d₆) δ 9.78 (br s, 1H), 8.94 (s, 1H), 8.64 (s, 1H), 8.47 (s, 1H), 8.39 (br s, 2H), 8.09 (br s, 1H), 7.56 (s, 1H), 3.85 (s, 3H), 3.81 (br s, 3H), 2.67-3.08 (m, 4H), 2.61 (s, 3H), 1.96 (br s, 7H), 1.56 (br s, 7H).

### Example 90. N-(2-(5-azaspiro[2.4]heptan-5-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

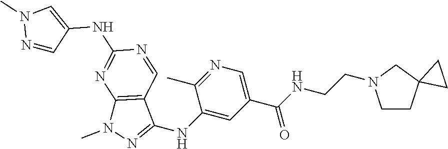 [SMILES: Cc1ncc(C(=O)NCCN2CCC3(CC3)C2)cc1Nc1nn(C)c2nc(Nc3cnn(C)c3)ncc12]

### Step a: 2-(5-azaspiro[2.4]heptan-5-yl)acetonitrile

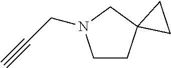

To a solution of 5-azaspiro[2.4]heptane hydrochloride (300 mg, 2.3 mmol) and potassium carbonate (683 mg, 4.9 mmol) in MeCN (15 mL) was added 2-Bromoacetonitrile (0.17 mL, 2.5 mmol) at room-temperature. The resulting mixture was stirred at 25° C. for 15 hours. The resulting mixture was quenched with water (20 ml) and extracted with ethyl acetate (40 ml*3). The organic extracts were dried over anhydrous Na₂SO₄ and filtered. The filtrate was concentrated under reduced pressure to give crude product, which was purified by column chromatography over silica gel (eluent: petroleum ether:ethyl acetate=4:1) to give the title compound 2-(5-azaspiro[2.4]heptan-5-yl)acetonitrile (180 mg, 59%) as a yellow oil. ¹H NMR (400 MHz, METHANOL-d₄) δ 3.74 (s, 2H), 2.86 (t, J=6.95 Hz, 2H), 2.63 (s, 2H), 1.85 (t, J=6.95 Hz, 2H), 0.58-0.66 (m, 2H), 0.52-0.58 (m, 2H).

### Step b: 2-(5-azaspiro[2.4]heptan-5-yl)ethanamine

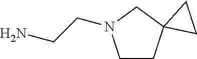

To a solution of 2-(5-azaspiro[2.4]heptan-5-yl)acetonitrile (170 mg, 1.3 mmol) in THF (5 mL) was added lithium aluminium hydride (57 mg, 1.5 mmol) by portions at 0° C. (ice/water). The resultant mixture was stirred at 20° C. for 1.5 h before quenched with water (0.1 mL) at 0° C. The reaction mixture was filtered. And the filtrate was concentrated to dryness under reduced pressure to afford the crude product 2-(5-azaspiro[2.4]heptan-5-yl)ethanamine (160 mg, 91%) as a colorless oil, which was used to the next step without further purification. ¹H NMR (400 MHz, METHANOL-d4) δ 2.74 (q, J=7.28 Hz, 4H), 2.49-2.60 (m, 4H), 1.82 (t, J=6.95 Hz, 2H), 0.48-0.64 (m, 4H).

### Step c: N-(2-(5-azaspiro[2.4]heptan-5-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

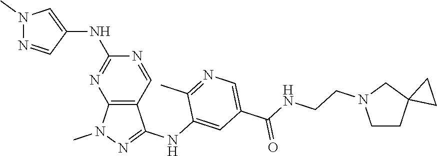 [SMILES: Cc1ncc(C(=O)NCCN2CCC3(CC3)C2)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (200 mg, 0.48 mmol), HATU (236 mg, 0.62 mmol) and N,N-diisopropylethylamine (0.32 mL, 1.9 mmol) in N,N-dimethylformamide (5 mL) was added 2-(5-azaspiro[2.4]heptan-5-yl)ethanamine (100 mg, 0.72 mmol). The mixture stirred at 25° C. for 2 h and then concentrated under vacuum to give the crude product, which was purified by preparative high-performance liquid chromatography over Column: Welch Xtimate C18 150*30 mm*5 um to give the title compound N-(2-(5-azaspiro[2.4]heptan-5-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (54 mg, 22%) as a yellow solid. LCMS (ESI): mass calcd. for C₂₅H₃₁N₁₁O, 501.6; m/z found, 502.3 [M+H]⁺. ¹H NMR (400 MHz, DMSO-d₆) δ 9.78 (br s, 1H), 8.94 (s, 1H), 8.66 (s, 1H), 8.44-8.55 (m, 2H), 8.39 (br s, 1H), 8.10 (br s, 1H), 7.56 (s, 1H), 3.85 (s, 3H), 3.81 (br s, 3H), 3.36-3.44 (m, 2H), 2.68 (t, J=6.90 Hz, 2H), 2.60 (s, 3H), 2.57 (br t, J=7.03 Hz, 2H), 2.47 (s, 2H), 1.71 (t, J=6.78 Hz, 2H), 0.50-0.54 (m, 2H), 0.45-0.49 (m, 2H).

### Example 91. N-(2-(cis-2,6-dimethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide

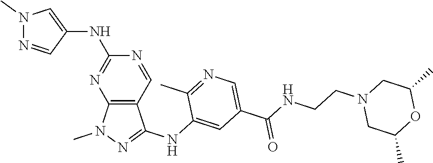

### Step a: 2-(cis-2,6-dimethylmorpholino)acetonitrile

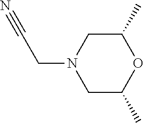

To a solution of cis-2,6-dimethylmorpholine (950 mg, 8.2 mmol) and potassium carbonate (3.42 g, 24.7 mmol) in acetonitrile (19 mL) was added 2-bromoacetonitrile (0.77 mL, 12.4 mmol) at room temperature. The resulting mixture was stirred at 50° C. for 16 h before cooling to room-temperature. The reaction mixture was filtered. The filtrate was concentrated under reduced pressure to give crude product, which was purified by column chromatography over silica gel (eluent: petroleum ether:ethyl acetate=3:1) to give the title compound 2-(cis-2,6-dimethylmorpholino)acetonitrile (2.5 g, 97%) as a colorless oil. LCMS (ESI): mass calcd. for C₈H₁₄N₂O, 154.2; m/z found, 155.2 [M+H]⁺. ¹H NMR (400 MHz, CDCl₃) δ 3.74-3.64 (m, 2H), 3.51 (s, 2H), 2.65 (s, 1H), 2.62 (s, 1H), 2.11 (t, J=10.5 Hz, 2H), 1.21 (s, 3H), 1.19 (s, 3H).

### Step b: 2-(cis-2,6-dimethylmorpholino)ethanamine

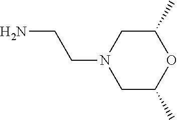

To a solution of 2-(cis-2,6-dimethylmorpholino)acetonitrile (1 g, 6.5 mmol) in THE (20 mL) was added lithium aluminium hydride (369 mg, 9.7 mmol) by portions at 0° C. (ice/water), and the resulting mixture was stirred at 25° C. for 1.5 h. After cooled to 0° C., the reaction mixture was quenched with water (369 mg) and filtered. The filtrate was then concentrated to dryness under reduced pressure to afford the crude product 2-(cis-2,6-dimethylmorpholino)ethanamine (870 mg, 85%) as a colorless oil. LCMS (ESI): mass calcd. for C₈H₁₈N₂O, 158.2; m/z found, 159.3[M+H]⁺. ¹H NMR (400 MHz, CDCl₃) δ 3.76-3.63 (m, 2H), 2.89-2.80 (m, 2H), 2.73 (br d, J=11.0 Hz, 2H), 2.48-2.40 (m, 2H), 1.77 (t, J=10.7 Hz, 2H), 1.18 (s, 3H), 1.17 (s, 3H).

### Step c: N-(2-(cis-2,6-dimethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide

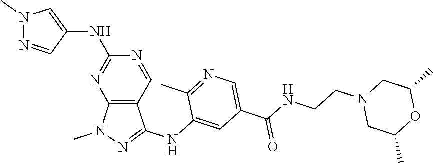

To a solution of 6-methyl-5-((1-methyl-6-((1-methyl-1H-pyrazol-4-yl)amino)-1H-pyrazolo[3,4-d]pyrimidin-3-yl)amino)nicotinic acid (130 mg, 0.34 mmol), HATU (192 mg, 0.50 mmol) and N,N-diisopropylethylamine (0.222 mL, 1.35 mmol) in N,N-dimethylformamide (5 mL) was added 2-(cis-2,6-dimethylmorpholino)ethanamine (69.2 mg, 0.44 mmol). The mixture stirred at 25° C. for 2 hours and then concentrated under vacuum to give the crude product, which was purified by column chromatography over silica gel (eluent: ethyl acetate:methanol=2:1) to give pure product, then the pure product was purified by preparative high-performance liquid chromatography over Column: Welch Xtimate C18 150*30 mm*5 um to give the title compound N-(2-(cis-2,6-dimethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide (58.6 mg, 32%) as a white solid. LCMS (ESI): mass calcd. for C₂₅H₃₃N₁₁O₂, 519.6; m/z found, 520.4 [M+H]⁺. ¹H NMR (400 MHz, METHANOL-d4) δ 8.87 (s, 1H), 8.76 (d, J=1.8 Hz, 1H), 8.47 (d, J=1.8 Hz, 1H), 8.11 (s, 1H), 7.67 (s, 1H), 3.93 (s, 3H), 3.87 (s, 3H), 3.73-3.65 (m, 2H), 3.57 (t, J=6.7 Hz, 2H), 2.89 (br d, J=10.8 Hz, 2H), 2.67 (s, 3H), 2.61 (t, J=6.7 Hz, 2H), 1.80 (t, J=10.8 Hz, 2H), 1.16 (s, 3H), 1.14 (s, 3H).

### Example 92. N-(2-(trans-2,6-dimethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide

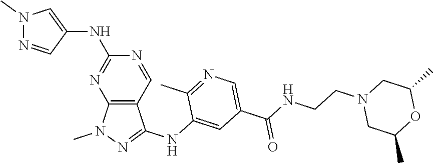

### Step a: 2-(trans-2,6-dimethylmorpholino)acetonitrile

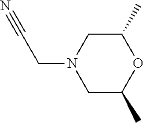

To a solution of trans-2,6-dimethylmorpholine (950 mg, 8.25 mmol) and potassium carbonate (3.42 g, 24.7 mmol) in acetonitrile (19 mL) was added 2-bromoacetonitrile (0.77 mL, 12.4 mmol) at room-temperature. The resulting mixture was stirred at 50° C. for 16 h before cooling to room-temperature. The reaction mixture was filtered. The filtrate was concentrated under reduced pressure to give crude product, which was purified by column chromatography over silica gel (eluent: petroleum ether:ethyl acetate=3:1) to give the title compound 2-(trans-2,6-dimethylmorpholino)acetonitrile (1.2 g, 94%) as a colorless oil. LCMS (ESI): mass calcd. for C₈H₁₄N₂O, 154.2; m/z found, 155.2 [M+H]⁺. ¹H NMR (400 MHz, CDCl₃) δ 4.14-4.04 (m, 2H), 3.58-3.43 (m, 2H), 2.66 (dd, J=3.0, 10.7 Hz, 2H), 2.32 (dd, J=5.7, 10.6 Hz, 2H), 1.28 (s, 3H), 1.27 (s, 3H).

### Step b: 2-(trans-2,6-dimethylmorpholino)ethanamine

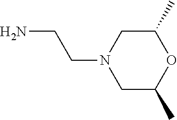

To a solution of 2-(trans-2,6-dimethylmorpholino)acetonitrile (1.2 g, 7.78 mmol) in THF (24 mL) was added lithium aluminium hydride (443.0 mg, 11.67 mmol) by portions at 0° C. (ice/water) and the resulting mixture was stirred at 25° C. for 1.5 h. After cooled to 0° C., the reaction mixture was quenched with water (443 mg) and filtered. The filtrate was then concentrated to dryness under reduced pressure to afford the crude product 2-(trans-2,6-dimethylmorpholino)ethanamine (1 g, 81%) as a colorless oil. LCMS (ESI): mass calcd. for C₈H₁₈N₂O, 158.2; m/z found, 159.2[M+H]⁺. ¹H NMR (400 MHz, CDCl₃) δ 4.04 (dt, J=3.4, 6.0 Hz, 2H), 2.88-2.76 (m, 2H), 2.49 (br dd, J=2.5, 11.0 Hz, 2H), 2.42-2.32 (m, 2H), 2.18 (br dd, J=5.6, 10.9 Hz, 2H), 1.27 (s, 3H), 1.25 (s, 3H).

### Step c: N-(2-(trans-2,6-dimethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamid

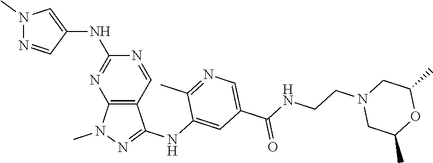

To a solution of 6-methyl-5-((1-methyl-6-((1-methyl-1H-pyrazol-4-yl)amino)-1H-pyrazolo[3,4-d]pyrimidin-3-yl)amino)nicotinic acid (160 mg, 0.41 mmol), HATU (236 mg, 0.62 mmol) and N,N-diisopropylethylamine (0.274 mL, 1.66 mmol) in N,N-dimethylformamide (5 mL) was added 2-(trans-2,6-dimethylmorpholino)ethanamine (85 mg, 0.54 mmol). The mixture stirred at 25° C. for 2 hours and then concentrated under vacuum to give the crude product, which was purified by column chromatography over silica gel (eluent: ethyl acetate:methanol=2:1) to give pure product, then the pure product was purified by preparative high-performance liquid chromatography over Column: Welch Xtimate C18 150*30 mm*5 um to give the title compound N-(2-(trans-2,6-dimethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide (60 mg, 27%) as a white solid. LCMS (ESI): mass calcd. for C₂₅H₃₃N₁₁O₂, 519.6; m/z found, 520.4 [M+H]⁺. ¹H NMR (400 MHz, METHANOL-d₄) δ 8.87 (s, 1H), 8.74 (d, J=1.8 Hz, 1H), 8.45 (d, J=1.8 Hz, 1H), 8.11 (s, 1H), 7.67 (s, 1H), 4.05-3.97 (m, 2H), 3.93 (s, 3H), 3.87 (s, 3H), 3.60-3.49 (m, 2H), 2.67 (s, 3H), 2.60-2.50 (m, 4H), 2.26 (br dd, J=5.6, 10.9 Hz, 2H), 1.22 (s, 3H), 1.21 (s, 3H).

### Example 93. N-(2-(cyclobutyl(3-methoxypropyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide

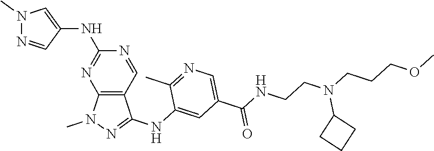

### Step a: N-(3-methoxypropyl)cyclobutanamine

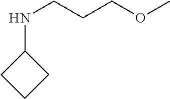

To a solution of 3-methoxypropan-1-amine (2 g, 22.4 mmol), cyclobutanone (1.6 g, 22.4 mmol) and acetic acid (1.413 mL, 24.7 mmol) in MeOH (40 mL) was added sodium cyanotrihydroborate (2.82 g, 44.9 mmol) by portions at 0° C. (ice/water). The resulting mixture was stirred at room temperature for 12 h. The reaction mixture was quenched with NaHCO₃ (20 mL) and extracted with ethyl acetate (60 mL×5). The organic layer was washed with brine, dried over anhydrous Na₂SO₄ and filtered. The filtrate was concentrated under reduced pressure to give the title compound N-(3-methoxypropyl)cyclobutanamine (350 mg, 11%) as a pale brown oil. LCMS (ESI): mass calcd. for C₈H₁₇NO, 143.2; m/z found, 144.3 [M+H]⁺. ¹H NMR (400 MHz, CDCl₃) δ 3.63 (t, J=5.5 Hz, 2H), 3.43-3.42 (m, 3H), 3.06 (s, 1H), 2.43-2.24 (m, 5H), 2.07-1.82 (m, 6H).

### Step b: 2-(cyclobutyl(3-methoxypropyl)amino)acetonitrile

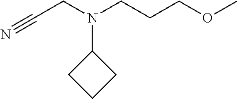

To a solution of N-(3-methoxypropyl)cyclobutanamine (300 mg, 2.10 mmol) and potassium carbonate (724 mg, 5.24 mmol) in acetonitrile (6 mL) was added 2-bromoacetonitrile (0.14 mL, 2.30 mmol) at room-temperature. The resulting mixture was stirred at 50° C. for 16 h before cooling to room-temperature. The reaction mixture was filtered. The residue was concentrated under reduced pressure to give crude product, which was purified by column chromatography over silica gel (eluent: petroleum ether:ethyl acetate=3:1) to give the title compound 2-(cyclobutyl(3-methoxypropyl)amino)acetonitrile (300 mg, 79%) as a colorless oil. LCMS (ESI): mass calcd. for C₁₀H₁₈N₂O, 182.2; m/z found, 183.2 [M+H]⁺. ¹H NMR (400 MHz, CDCl₃) δ 3.54 (br s, 2H), 3.44 (t, J=6.3 Hz, 2H), 3.36 (s, 3H), 3.22-3.04 (m, 1H), 2.52 (br s, 2H), 2.17-2.07 (m, 2H), 1.80 (br d, J=6.6 Hz, 2H), 1.73 (br d, J=5.7 Hz, 4H).

### Step c: N¹-cyclobutyl-N¹-(3-methoxypropyl)ethane-1,2-diamine

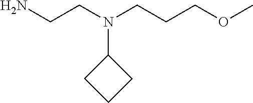

To a solution of 2-(cyclobutyl(3-methoxypropyl)amino)acetonitrile (300 mg, 1.65 mmol) in THF (6 mL) was added lithium aluminium hydride (93.7 mg, 2.47 mmol) by portions at 0° C. (ice/water) and the resulting mixture was stirred at 25° C. for 1.5 h. After cooled to 0° C., the reaction mixture was quenched with water (93.7 mg) and filtered. The filtrate was then concentrated to dryness under reduced pressure to afford the crude product N1-cyclobutyl-N1-(3-methoxypropyl)ethane-1,2-diamine (220 mg, 72%) as a colorless oil. LCMS (ESI): mass calcd. for C₁₀H₂₂N₂O, 186.2; m/z found, 187.2[M+H]⁺. ¹H NMR (400 MHz, CDCl₃) δ 3.41 (t, J=6.4 Hz, 2H), 3.35 (s, 3H), 3.17-3.06 (m, 1H), 2.75 (t, J=6.4 Hz, 2H), 2.53-2.43 (m, 4H), 2.07-2.00 (m, 2H), 1.94-1.83 (m, 2H), 1.73-1.60 (m, 6H).

### Step d: N-(2-(cyclobutyl(3-methoxypropyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide

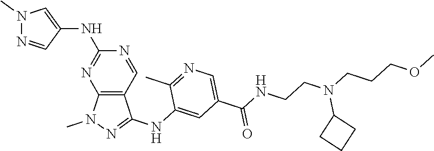

To a solution of 6-methyl-5-((1-methyl-6-((1-methyl-1H-pyrazol-4-yl)amino)-1H-pyrazolo[3,4-d]pyrimidin-3-yl)amino)nicotinic acid (140 mg, 0.33 mmol), HATU (190 mg, 0.50 mmol) and N,N-diisopropylethylamine (0.220 mL, 1.33 mmol) in N,N-dimethylformamide (5 mL) was added Ni-cyclobutyl-N¹-(3-methoxypropyl)ethane-1,2-diamine (80.8 mg, 0.43 mmol). The mixture stirred at 25° C. for 2 hours and then concentrated under vacuum to give the crude product, which was purified by column chromatography over silica gel (eluent: ethyl acetate:methanol=2:1) to give pure product, then the pure product was purified by preparative high-performance liquid chromatography over Column: Welch Xtimate C18 150*30 mm*5 um to give the title compound: N-(2-(cyclobutyl(3-methoxypropyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide (71 mg, 38%) as a white solid. LCMS (ESI): mass calcd. for C₂₇H₃₇N₁₁O₂, 547.6; m/z found, 548.4 [M+H]⁺. ¹H NMR (400 MHz, METHANOL-d₄) δ 8.87 (s, 1H), 8.76 (d, J=1.9 Hz, 1H), 8.46 (d, J=1.9 Hz, 1H), 8.11 (s, 1H), 7.67 (s, 1H), 3.93 (s, 3H), 3.87 (s, 3H), 3.49 (t, J=7.0 Hz, 2H), 3.42 (t, J=6.1 Hz, 2H), 3.30 (s, 3H), 3.22 (br t, J=7.5 Hz, 1H), 2.69 (t, J=7.0 Hz, 2H), 2.66 (s, 3H), 2.62-2.58 (m, 2H), 2.11-2.05 (m, 2H), 1.95-1.88 (m, 2H), 1.79-1.73 (m, 2H), 1.72-1.65 (m, 2H).

### Example 94. N-(2-(trans-3,5-dimethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide

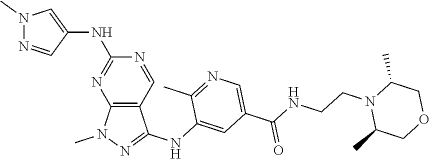 [SMILES: Cc1ncc(C(=O)NCCN2[C@H](C)COC[C@H]2C)cc1Nc1nn(C)c2nc(Nc3cnn(C)c3)ncc12]

### Step a: 2-(trans-3,5-dimethylmorpholino)acetonitrile

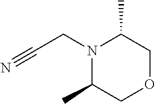

To a solution of trans-3,5-dimethylmorpholine hydrochloride (450 mg, 2.97 mmol) and potassium carbonate (1.02 g, 7.4 mmol) in acetonitrile (10 mL) was added 2-bromoacetonitrile (203 uL, 3.3 mmol) at room temperature. The resulting mixture was stirred at 50° C. for 12 h before cooling to room-temperature. The reaction mixture was filtered. The residue was washed with ethyl acetate (10 mL×3) and the filtrate was concentrated under reduced pressure to give crude product, which was purified by column chromatography over silica gel (eluent: petroleum ether:ethyl acetate=3:1) to give the title compound 2-(trans-3,5-dimethylmorpholino)acetonitrile (450 mg, 98%) as a yellow oil. LCMS (ESI): mass calcd. for C₈H₁₄N₂O, 154.2; m/z found, 155.2 [M+H]⁺. ¹H NMR (400 MHz, CDCl₃) δ 3.71-3.61 (m, 3H), 3.40-3.26 (m, 3H), 2.95 (dquin, J=3.2, 6.3 Hz, 2H), 1.07 (s, 3H), 1.05 (s, 3H).

### Step b: 2-(trans-3,5-dimethylmorpholino)ethanamine

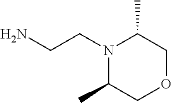

To a solution of 2-(trans-3,5-dimethylmorpholino)acetonitrile (420 mg, 2.72 mmol) in THF (8 mL) was added lithium aluminium hydride (207 mg, 5.4 mmol) by portions at 0° C. (ice/water), and the resulting mixture was stirred for 1.5 h at 25° C. After cooled to 0° C., the reaction mixture was quenched with water (207 mg) and filtered. The filtrate was then concentrated to dryness under reduced pressure to afford the crude product 2-(trans-3,5-dimethylmorpholino)ethanamine (370 mg, 86%) as a colorless oil. LCMS (ESI): mass calcd. for C₈H₁₈N₂O, 158.2; m/z found, 159.3[M+H]⁺. ¹H NMR (400 MHz, CDCl₃) δ 3.65 (dd, J=3.1, 10.8 Hz, 2H), 3.36 (dd, J=6.0, 10.8 Hz, 2H), 2.88-2.78 (m, 2H), 2.77-2.63 (m, 3H), 2.43-2.32 (m, 1H), 0.96 (d, J=6.4 Hz, 6H).

### Step c: N-(2-(trans-3,5-dimethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide

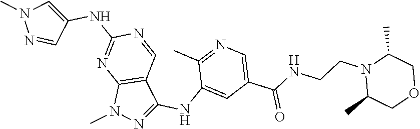 [SMILES: Cc1ncc(C(=O)NCCN2[C@H](C)COC[C@H]2C)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (130 mg, 0.34 mmol), HATU (192 mg, 0.5 mmol) and N,N-diisopropylethylamine (223 ul, 1.34 mmol) in N,N-dimethylformamide (5 mL) was added 2-(trans-3,5-dimethylmorpholino)ethanamine (69 mg, 0.44 mmol). The mixture stirred at 25° C. for 2 hours and then concentrated under vacuum to give the crude product, which was purified by preparative high-performance liquid chromatography over column: Phenomenex Gemini-NX 80*40 mm*3 um to give the title compound N-(2-(trans-3,5-dimethylmorpholino)ethyl)-6-methyl-5-((1-methyl-6-((1-methyl-1H-pyrazol-4-yl)amino)-1H-pyrazolo[3,4-d]pyrimidin-3-yl)amino)nicotinamide (41.3 mg, 22%) as a white solid. LCMS (ESI): mass calcd. for C₂₅H₃₃N₁₁O₂, 519.6; m/z found, 502.1 [M+H]⁺. ¹H NMR (400 MHz, METHANOL-d₄) δ 8.86 (s, 1H), 8.74 (d, J=1.8 Hz, 1H), 8.44 (d, J=1.8 Hz, 1H), 8.10 (s, 1H), 7.65 (s, 1H), 3.91 (s, 3H), 3.86 (s, 3H), 3.69 (d, J=2.9 Hz, 1H), 3.66 (d, J=2.9 Hz, 1H), 3.53-3.42 (m, 2H), 3.37 (dd, J=6.0, 11.0 Hz, 2H), 2.97-2.90 (m, 3H), 2.65 (s, 3H), 2.57 (ddd, J=5.5, 7.7, 13.1 Hz, 1H), 1.05 (d, J=6.4 Hz, 6H).

### Example 95. N-(2-(cyclopentyl(2-methoxyethyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide

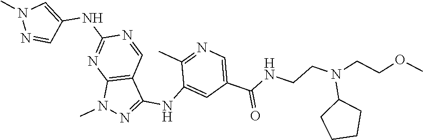

### Step a: N-(2-methoxyethyl)cyclopentanamine

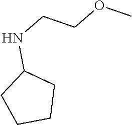

To a solution of 2-methoxyethanamine (2 g, 26.6 mmol) in methanol (10 mL) was added cyclopentanone (2.3 g, 27.9 mmol), acetic acid (1.7 mL, 29.3 mmol) and NaBH₃CN (3.3 g, 53.2 mmol). The mixture was stirred at room temperature for 2 hours. Saturated aqueous NaHCO₃ (15 mL) was added and extracted with the solution (DCM/MeOH=6/1, 20 mL*8). The organic layer was washed with brine, dried with anhydrous Na₂SO₄, filtered and the filtrate was evaporated in vacuo to give the crude product N-(2-methoxyethyl)cyclopentanamine as a yellow liquid. MS (ESI): mass calcd. for C₈H₁₇NO, 143.227; m/z found, 143.9 [M+H]⁺. ¹H NMR (400 MHz, CDCl₃) δ 5.88 (br s, 1H), 3.51 (t, J=5.07 Hz, 2H), 3.24-3.32 (m, 4H), 2.95 (t, J=5.07 Hz, 2H), 1.80-1.97 (m, 2H), 1.67 (br d, J=6.84 Hz, 2H), 1.43-1.57 (m, 4H).

### Step b: 2-(cyclopentyl(2-methoxyethyl)amino)acetonitrile

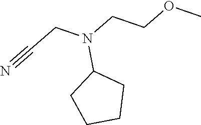

To a solution of N-(2-methoxyethyl)cyclopentanamine (2 g, 14.0 mmol) and potassium carbonate (4.8 g, 34.9 mmol) in acetonitrile (20 mL) was added 2-bromoacetonitrile (0.96 mL, 15.4 mmol) at room temperature. The resulting mixture was stirred at room temperature overnight. The reaction mixture was filtered and the filtrate was concentrated in vacuo to give crude product, which was purified by column chromatography over silica gel (eluent: petroleum ether:ethyl acetate=1:1) to give the title compound 2-(cyclopentyl(2-methoxyethyl)amino)acetonitrile (1.7 g, 67%) as a colorless liquid. MS (ESI): mass calcd. for C₁₀H₁₈N₂O, 182.263; m/z found, 183.0 [M+H]⁺. ¹H NMR (400 MHz, CDCl₃) δ 3.69 (s, 2H), 3.43 (t, J=5.19 Hz, 2H), 3.29 (s, 3H), 2.86 (br t, J=7.33 Hz, 1H), 2.73 (t, J=5.07 Hz, 2H), 1.79-1.90 (m, 2H), 1.60-1.70 (m, 2H), 1.49-1.57 (m, 2H), 1.33 (br dd, J=10.37, 8.58 Hz, 2H).

### Step c: N¹-cyclopentyl-N-(2-methoxyethyl)ethane-1,2-diamine

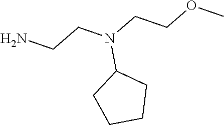

To a solution of 2-(cyclopentyl(2-methoxyethyl)amino)acetonitrile (1.7 g, 9.3 mmol) in THF (20 mL) was added lithium aluminium hydride (425 mg, 11.2 mmol) by portions at 0° C. (ice/water), and the resulting mixture was stirred at 25° C. for 1.5 h. After cooled to 0° C., the reaction mixture was quenched with water (425 mg) and filtered. The filtrate was then concentrated to dryness under reduced pressure to afford the crude product Ni-cyclopentyl-N¹-(2-methoxyethyl)ethane-1,2-diamine (1.3 g, 75%) as a colorless liquid. ¹H NMR (400 MHz, DMSO-d₆) δ 3.32-3.35 (m, 4H), 3.22 (s, 3H), 3.03 (quin, J=8.05 Hz, 1H), 2.57 (t, J=6.56 Hz, 2H), 2.40-2.45 (m, 2H), 1.63-1.74 (m, 2H), 1.57 (dt, J=7.81, 3.84 Hz, 2H), 1.39-1.51 (m, 2H), 1.25-1.36 (m, 2H), 1.20 (br t, J=6.44 Hz, 2H).

### Step d: N-(2-(cyclopentyl(2-methoxyethyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide

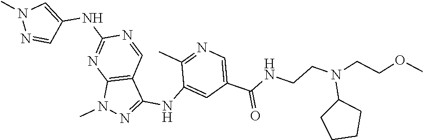

To a solution of 6-methyl-5-((1-methyl-6-((1-methyl-1H-pyrazol-4-yl)amino)-1H-pyrazolo[3,4-d]pyrimidin-3-yl)amino)nicotinic acid (150 mg, 0.36 mmol), HATU (177 mg, 0.46 mmol) and N,N-diisopropylethylamine (240 ul, 1.43 mmol) in N,N-dimethylformamide (5 mL) was added Ni-cyclopentyl-N¹-(2-methoxyethyl)ethane-1,2-diamine (80 mg, 0.43 mmol). The mixture stirred at 25° C. for 1 hour and then concentrated under vacuum to give the crude product, which was purified by preparative high-performance liquid chromatography over column: Phenomenex Gemini NX C18 150*40 mm*5 um to give the title compound N-(2-(cyclopentyl(2-methoxyethyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide (92.0 mg, 42%) as a white solid. LCMS (ESI): mass calcd. for C₂₇H₃₇N₁₁O₂, 547.655; m/z found, 548.3 [M+H]⁺. ¹H NMR (400 MHz, DMSO-d₆) δ 9.78 (br s, 1H), 8.94 (s, 1H), 8.64 (s, 1H), 8.45-8.50 (m, 2H), 8.39 (s, 1H), 8.15 (s, 1H), 7.56 (s, 1H), 3.85 (s, 3H), 3.81 (br s, 2H), 3.43 (br t, J=6.15 Hz, 3H), 3.31-3.39 (m, 3H), 3.11-3.21 (m, 2H), 2.66-2.79 (m, 5H), 2.60 (s, 3H), 1.74-1.80 (m, 2H), 1.56-1.62 (m, 2H), 1.43-1.50 (m, 2H), 1.32-1.40 (m, 2H).

### Example 96. N-(2-(2,2-dimethylpyrrolidin-1-yl)ethyl)-6-methyl-5-((1-methyl-6-((1-methyl-1H-pyrazol-4-yl)amino)-1H-pyrazolo[4,3-c]pyridin-3-yl)amino)nicotinamide

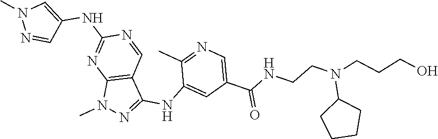 [SMILES: Cc1ncc(C(=O)NCCN(CCCO)C2CCCC2)cc1Nc1nn(C)c2nc(Nc3cnn(C)c3)ncc12]

### Step a: 3-(cyclopentylamino)propan-1-ol

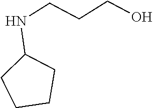

To a solution of 3-aminopropan-1-ol (500 mg, 6.7 mmol) in methanol (7 mL) was added cyclopentanone (616 mg, 7.3 mmol), acetic acid (0.42 mL, 7.3 mmol) and NaBH₃CN (837 mg, 13.3 mmol) The reaction mixture was stirred at room temperature for 12 hours. MS showed the completion of the reaction. Saturated aqueous NaHCO₃ (15 mL) was added and extracted with the solution (DCM/MeOH=6/1, 20 mL*8). The organic layer was washed with brine, dried with Na₂SO₄, filtered and the filtrate was evaporated in vacuo to give the crude product 3-(cyclopentylamino)propan-1-ol (400 mg, 42%) as a pale brown liquid. ¹H NMR (400 MHz, CDCl₃) δ 3.96 (br t, J=5.4 Hz, 2H), 3.52 (td, J=6.8, 14.1 Hz, 1H), 3.28-3.20 (m, 2H), 2.13-2.08 (m, 2H), 1.89-1.68 (m, 8H).

### Step b: 2-(cyclopentyl(3-hydroxypropyl)amino)acetonitrile

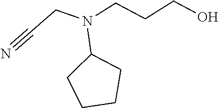 [SMILES: N#CCN(CCCO)C1CCCC1]

To a solution of 3-(cyclopentylamino) propan-1-ol (370 mg, 2.6 mmol) and potassium carbonate (785 mg, 5.7 mmol) in acetonitrile (10 mL) was added 2-bromoacetonitrile (340 mg, 2.8 mmol) at room temperature. The resulting mixture was stirred at room temperature overnight. The reaction mixture was filtered and the filtrate was concentrated in vacuo to give crude product, which was purified by column chromatography over silica gel (eluent: petroleum ether:ethyl acetate=1:1) to give the title compound 2-(cyclopentyl(3-hydroxypropyl)amino)acetonitrile (260 mg, 55%) as a pale brown oil. ¹H NMR (400 MHz, CDCl₃) δ 3.72 (t, J=5.4 Hz, 3H), 3.75-3.68 (m, 1H), 2.97-2.81 (m, 3H), 1.92 (br d, J=4.8 Hz, 2H), 1.76-1.51 (m, 8H).

### Step c: 3-((2-aminoethyl)(cyclopentyl)amino)propan-1-ol

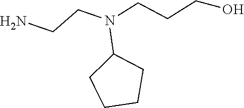 [SMILES: NCCN(CCCO)C1CCCC1]

To a solution of 2-(cyclopentyl(3-hydroxypropyl)amino)acetonitrile (260 mg, 1.4 mmol) in THF (10 mL) was added lithium aluminium hydride (81.2 mg, 2.1 mmol) by portions at 0° C. (ice/water), and the resulting mixture was stirred at 25° C. for 1.5 h. After cooled to 0° C., the reaction mixture was quenched with water (81.2 mg) and filtered. The filtrate was then concentrated to dryness under reduced pressure to afford the crude product 3-((2-aminoethyl)(cyclopentyl)amino)propan-1-ol (210 mg, 79%) as a pale brown oil.

### Step d: N-(2-(cyclopentyl(3-hydroxypropyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide

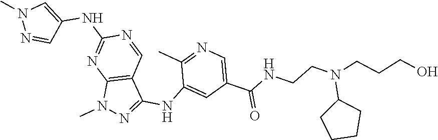 [SMILES: Cc1ncc(C(=O)NCCN(CCCO)C2CCCC2)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (150 mg, 0.37 mmol), HATU (251 mg, 0.66 mmol) and N,N-diisopropylethylamine (190 ul, 1.1 mmol) in N,N-dimethylformamide (10 mL) was added 3-((2-aminoethyl)(cyclopentyl)amino)propan-1-ol (205 mg, 1.1 mmol). The mixture stirred at 25° C. for 1 hour and then concentrated under vacuum to give the crude product, which was purified by preparative high-performance liquid chromatography over column: Phenomenex Gemini-NX C18 75*30 mm*3 um to give the title compound N-(2-(cyclopentyl(3-hydroxypropyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide (54 mg, 26%) as a pale brown solid. LCMS (ESI): mass calcd. for C₂₇H₃₇N₁₁O₂, 547.655; m/z found, 548.4 [M+H]⁺. ¹H NMR (400 MHz, METHANOL-d₄) δ 8.87 (s, 1H), 8.76 (d, J=1.8 Hz, 1H), 8.46 (d, J=1.8 Hz, 1H), 8.11 (s, 1H), 7.67 (s, 1H), 3.93 (s, 3H), 3.87 (s, 3H), 3.64 (t, J=6.0 Hz, 2H), 3.53 (t, J=7.1 Hz, 2H), 3.16 (quin, J=8.1 Hz, 1H), 2.78 (td, J=7.4, 14.7 Hz, 4H), 2.66 (s, 3H), 1.90 (br d, J=6.8 Hz, 2H), 1.81-1.74 (m, 2H), 1.73-1.66 (m, 2H), 1.64-1.55 (m, 2H), 1.51-1.40 (m, 2H).

### Example 97. N³-(2-chloro-6-methylphenyl)-N⁶-(2-(4-(2-fluoroethyl)piperazin-1-yl)-6-methylpyridin-4-yl)-1-methyl-1H-pyrazolo[3,4-d]pyrimidine-3,6-diamine

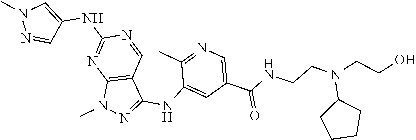 [SMILES: Cc1ncc(C(=O)NCCN(CCO)C2CCCC2)cc1Nc1nn(C)c2nc(Nc3cnn(C)c3)ncc12]

### Step a: 2-(cyclopentyl(2-hydroxyethyl)amino)acetonitrile

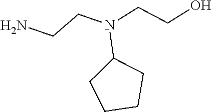 [SMILES: NCCN(CCO)C1CCCC1]

To a solution of 2-(cyclopentylamino)ethanol (700 mg, 5.4 mmol) and potassium carbonate (1.65 g, 11.9 mmol) in acetonitrile (14 mL) was added 2-bromoacetonitrile (715 mg, 6.0 mmol) at room temperature. The resulting mixture was stirred at room temperature overnight. The reaction mixture was filtered and the filtrate was concentrated in vacuo to give crude product, which was purified by column chromatography over silica gel (eluent: petroleum ether:ethyl acetate=1:1) to give the title compound 2-(cyclopentyl(2-hydroxyethyl)amino)acetonitrile (849 mg, 93%) as a pale brown oil. ¹H NMR (400 MHz, CDCl₃) δ 3.63-3.54 (m, 4H), 3.01-2.91 (m, 1H), 2.74 (t, J=5.3 Hz, 2H), 2.06 (br s, 1H), 1.93-1.83 (m, 2H), 1.71-1.61 (m, 2H), 1.60-1.50 (m, 2H), 1.38-1.26 (m, 2H).

### Step b: 2-((2-aminoethyl)(cyclopentyl)amino)ethanol

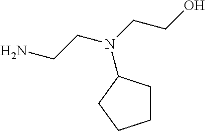 [SMILES: NCCN(CCO)C1CCCC1]

To a solution of 2-(cyclopentyl(2-hydroxyethyl)amino)acetonitrile (849 mg, 5.0 mmol) in THF (20 mL) was added lithium aluminium hydride (287.3 mg, 7.6 mmol) by portions at 0° C. (ice/water), and the resulting mixture was stirred at 25° C. for 1.5 h. After cooled to 0° C., the reaction mixture was quenched with water (287.3 mg) and filtered. The filtrate was then concentrated to dryness under reduced pressure to afford the crude product 2-((2-aminoethyl)(cyclopentyl)amino)ethanol (800 mg, 92%) as a pale brown oil.

### Step c: N-(2-(cyclopentyl(2-hydroxyethyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide

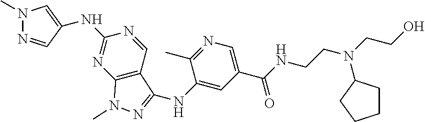 [SMILES: Cc1ncc(C(=O)NCCN(CCO)C2CCCC2)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (130 mg, 0.32 mmol), HATU (218 mg, 0.57 mmol) and N,N-diisopropylethylamine (166 ul, 0.95 mmol) in N,N-dimethylformamide (8 mL) was added 2-((2-aminoethyl)(cyclopentyl)amino)ethanol (98.6 mg, 0.57 mmol). The mixture stirred at 25° C. for 1 hour and then concentrated under vacuum to give the crude product, which was purified by preparative high-performance liquid chromatography over column: Boston Green ODS 150*30 mm*5 um to give the title compound N-(2-(cyclopentyl(2-hydroxyethyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide (40 mg, 21%) as a brown solid. LCMS (ESI): mass calcd. for C₂₆H₃₅N₁₁O₂, 533.629; m/z found, 534.3 [M+H]⁺. ¹H NMR (400 MHz, METHANOL-d₄) δ 8.89 (s, 1H), 8.79 (d, J=1.8 Hz, 1H), 8.49 (d, J=1.9 Hz, 1H), 8.43 (s, 1H), 8.10 (s, 1H), 7.69 (s, 1H), 3.93 (s, 3H), 3.91-3.85 (m, 6H), 3.81-3.75 (m, 1H), 3.78 (br t, J=6.3 Hz, 1H), 3.39 (br t, J=6.3 Hz, 2H), 3.32-3.28 (m, 2H), 2.68 (s, 2H), 2.70-2.66 (m, 1H), 2.15 (br s, 2H), 1.84 (br s, 2H), 1.77-1.67 (m, 4H).

### Example 98. 5-((6-((3-acetamidophenyl)amino)-1-methyl-1H-pyrazolo[3,4-d]pyrimidin-3-yl)amino)-N-(2-(2,2-dimethylpyrrolidin-1-yl)ethyl)-6-methylnicotinamide

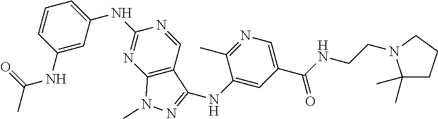 [SMILES: CC(=O)Nc1cccc(Nc2ncc3c(Nc4cc(C(=O)NCCN5CCCC5(C)C)cnc4C)nn(C)c3n2)c1]

### Step a: 5-((6-chloro-1-methyl-1H-pyrazolo[3,4-d]pyrimidin-3-yl)amino)-N-(2-(2,2-dimethylpyrrolidin-1-yl)ethyl)-6-methylnicotinamide

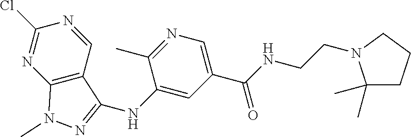 [SMILES: Cc1ncc(C(=O)NCCN2CCCC2(C)C)cc1Nc1nn(C)c2nc(Cl)ncc12]

To a solution of 2-(2,2-dimethylpyrrolidin-1-yl)ethanamine (534 mg, 3.75 mmol) in 1,2-dichloroethane (10 mL) was added trimethylaluminium (1.87 mL, 3.75 mmol, 2 M in toluene) by portions at 0° C. (ice/water) and the resulting mixture was stirred at 0° C. for 30 minutes. Then methyl 5-((6-chloro-1-methyl-1H-pyrazolo[3,4-d]pyrimidin-3-yl)amino)-6-methylnicotinate (500 mg, 1.5 mmol) was added and the resulting mixture was stirred at 60° C. for 3 hours. After cooled to 0° C., the reaction mixture was quenched with HCl aqueous (5 mL, 1 M) and filtered. The filtrate was then concentrated to dryness under reduced pressure to afford the crude product, which was purified by preparative high-performance liquid chromatography over column: Boston Uni C18 40*150*5 um to give the title compound 5-((6-chloro-1-methyl-1H-pyrazolo[3,4-d]pyrimidin-3-yl)amino)-N-(2-(2,2-dimethylpyrrolidin-1-yl)ethyl)-6-methylnicotinamide (470 mg, 71%) as a yellow solid. LCMS (ESI): mass calcd. for C₂₁H₂₇ClN₈O, 442.9; m/z found, 443.9[M+H]⁺.

### Step b: 5-((6-((3-acetamidophenyl)amino)-1-methyl-1H-pyrazolo[3,4-d]pyrimidin-3-yl)amino)-N-(2-(2,2-dimethylpyrrolidin-1-yl)ethyl)-6-methylnicotinamide

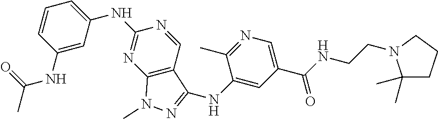 [SMILES: CC(=O)Nc1cccc(Nc2ncc3c(Nc4cc(C(=O)NCCN5CCCC5(C)C)cnc4C)nn(C)c3n2)c1]

To a solution of 5-((6-chloro-1-methyl-1H-pyrazolo[3,4-d]pyrimidin-3-yl)amino)-N-(2-(2,2-dimethylpyrrolidin-1-yl)ethyl)-6-methylnicotinamide (200 mg, 0.45 mmol), 3-aminoacetanilide (102 mg, 0.67 mmol), cesium carbonate (441 mg, 1.35 mmol) and Brettphos (48 mg, 0.09 mmol) in DMF (20 mL) was added Brettphos-Pd-G3 (82 mg, 0.09 mmol) under N₂. The resulting mixture was stirred at 95° C. under N₂ for 12 h before cooled to 25° C. Then reaction mixture was concentrated under vacuum to give the crude product, which was purified by column chromatography over silica gel (eluent: Petroleum ether/ethyl acetate=100:0 to 0:100˜ dichloromethane/methanol=100:0 to 50:50) to give crude product, then the crude product was purified by preparative high-performance liquid chromatography over column: Welch Xtimate C18 150*30 mm*5 um to give the title compound 5-((6-((3-acetamidophenyl)amino)-1-methyl-1H-pyrazolo[3,4-d]pyrimidin-3-yl)amino)-N-(2-(2,2-dimethylpyrrolidin-1-yl)ethyl)-6-methylnicotinamide (13.9 mg, 5%) as a white solid. LCMS (ESI): mass calcd. for C₂₉H₃₆N₁₀O₂, 556.6; m/z found, 557.4 [M+H]⁺. 1H NMR (400 MHz, METHANOL-d₄) δ 8.93 (s, 1H), 8.77 (s, 1H), 8.49 (s, 2H), 7.42 (br d, J=8.5 Hz, 1H), 7.27 (t, J=7.9 Hz, 1H), 7.12 (br d, J=8.6 Hz, 1H), 3.92 (s, 3H), 3.55 (br t, J=6.3 Hz, 2H), 3.00 (br s, 2H), 2.76 (br s, 2H), 2.68 (s, 3H), 2.17 (s, 3H), 1.87 (br d, J=7.7 Hz, 2H), 1.75 (br d, J=8.1 Hz, 2H), 1.10 (br s, 6H).

### Example 99. 5-((6-((5-acetamidopyridin-3-yl)amino)-1-methyl-1H-pyrazolo[3,4-d]pyrimidin-3-yl)amino)-N-(2-(2,2-dimethylpyrrolidin-1-yl)ethyl)-6-methylnicotinamide

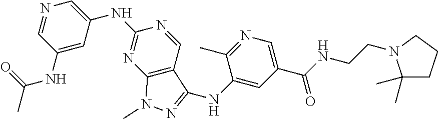 [SMILES: CC(=O)Nc1cncc(Nc2ncc3c(Nc4cc(C(=O)NCCN5CCCC5(C)C)cnc4C)nn(C)c3n2)c1]

### Step a: 5-((6-((5-acetamidopyridin-3-yl)amino)-1-methyl-1H-pyrazolo[3,4-d]pyrimidin-3-yl)amino)-N-(2-(2,2-dimethylpyrrolidin-1-yl)ethyl)-6-methylnicotinamide

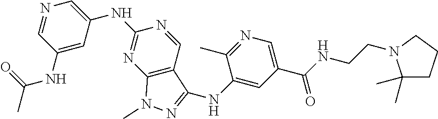 [SMILES: CC(=O)Nc1cncc(Nc2ncc3c(Nc4cc(C(=O)NCCN5CCCC5(C)C)cnc4C)nn(C)c3n2)c1]

To a solution of 5-((6-chloro-1-methyl-1H-pyrazolo[3,4-d]pyrimidin-3-yl)amino)-N-(2-(2,2-dimethylpyrrolidin-1-yl)ethyl)-6-methylnicotinamide (400 mg, 0.9 mmol), N-(5-aminopyridin-3-yl)acetamide (500 mg, 3.31 mmol), potassium phosphate (575 mg, 2.71 mmol) and Brettphos (97 mg, 0.18 mmol) in DMF (40 mL) was added Brettphos-Pd-G3 (164 mg, 0.18 mmol) under N₂. The resulting mixture was stirred at 100° C. under N₂ for 12 h before cooled to 25° C. Then reaction mixture was concentrated under vacuum to give the crude product, which was purified by preparative high-performance liquid chromatography over column: Boston Uni C18 40*150*5 um to give the title compound 5-((6-((5-acetamidopyridin-3-yl)amino)-1-methyl-1H-pyrazolo[3,4-d]pyrimidin-3-yl)amino)-N-(2-(2,2-dimethylpyrrolidin-1-yl)ethyl)-6-methylnicotinamide as formate salt (51.5 mg, 9%) as a yellow solid. LCMS (ESI): mass calcd. for C₂₈H₃₅N₁₁O₂, 557.6; m/z found, 558.4 [M+H]⁺. ¹H NMR (400 MHz, METHANOL-d₄) δ 8.96 (s, 1H), 8.82 (d, J=1.8 Hz, 1H), 8.77 (d, J=2.5 Hz, 1H), 8.53 (br s, 1H), 8.50 (d, J=1.8 Hz, 1H), 8.30 (dd, J=2.7, 9.0 Hz, 1H), 8.05 (br d, J=9.2 Hz, 1H), 3.87 (s, 3H), 3.71 (br t, J=6.4 Hz, 2H), 3.51-3.38 (m, 2H), 3.17 (br s, 2H), 2.70-2.67 (m, 3H), 2.20 (s, 3H), 2.13-2.03 (m, 2H), 2.01-1.93 (m, 2H), 1.33 (s, 6H).

### Example 100. 5-((6-((5-acetamidopyridin-3-yl)amino)-1-methyl-1H-pyrazolo[3,4-d]pyrimidin-3-yl)amino)-N-(2-(2,2-dimethylpyrrolidin-1-yl)ethyl)-6-methylnicotinamide

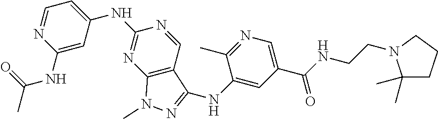 [SMILES: CC(=O)Nc1cc(Nc2ncc3c(Nc4cc(C(=O)NCCN5CCCC5(C)C)cnc4C)nn(C)c3n2)ccn1]

### Step a: tert-butyl (2-acetamidopyridin-4-yl)carbamate

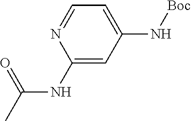 [SMILES: CC(=O)Nc1cc(NC(=O)OC(C)(C)C)ccn1]

To a solution of N-(4-bromopyridin-2-yl)acetamide (500 mg, 2.33 mmol), tert-butyl carbamate (327 mg, 2.79 mmol), cesium carbonate (1.06 g, 3.26 mmol) and Brettphos (332 mg, 0.70 mmol) in DMF (20 mL) was added tris(dibenzylideneacetone)dipalladium (213 mg, 0.23 mmol) under N₂. The resulting mixture was stirred at 80° C. under N₂ for 12 h before cooled to 25° C. Then reaction mixture was concentrated under vacuum to give the crude product, which was purified by preparative high-performance by column chromatography over silica gel (eluent: petroleum ether/ethyl acetate=100:0 to 50:50) to give tert-butyl (2-acetamidopyridin-4-yl)carbamate (520 mg, 87%) as light yellow solid. LCMS (ESI): mass calcd. for C₁₂H₁₇N₃O₃, 251.2; m/z found, 252.1 [M+H]⁺. ¹H NMR (400 MHz, METHANOL-d₄) δ 8.15-8.03 (m, 2H), 7.30 (dd, J=1.7, 5.7 Hz, 1H), 2.18 (s, 3H), 1.54 (s, 9H).

### Step b: N-(4-aminopyridin-2-yl)acetamide

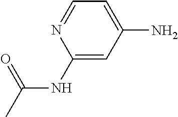

To a solution of tert-butyl (2-acetamidopyridin-4-yl)carbamate (400 mg, 2.325 mmol) in 1,2-dichloroethane (5 mL) was added trifluoroacetic acid (15 mL) under N₂. The resulting mixture was stirred at 25° C. under N₂ for 2 hours. Then reaction mixture was quenched with saturated aqueous NaHCO₃ (8 mL) and extracted with ethyl acetate (20 mL*5). The organic layer was washed with brine, dried over anhydrous Na₂SO₄, filtered and concentrated in vacuum to give N-(4-aminopyridin-2-yl)acetamide (270 mg, 90%) as orange solid. LCMS (ESI): mass calcd. for C₇H₉N₃O, 151.1; m/z found, 152.2 [M+H]⁺. ¹H NMR (400 MHz, METHANOL-d₄) δ 7.63 (d, J=5.8 Hz, 1H), 7.14 (br s, 1H), 6.24 (dd, J=2.1, 5.9 Hz, 1H), 2.03 (s, 3H).

### Step c: 5-((6-((2-acetamidopyridin-4-yl)amino)-1-methyl-1H-pyrazolo[3,4-d]pyrimidin-3-yl)amino)-N-(2-(2,2-dimethylpyrrolidin-1-yl)ethyl)-6-methylnicotinamide

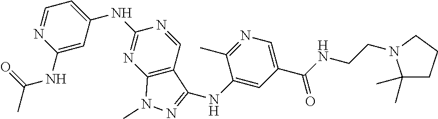 [SMILES: CC(=O)Nc1cc(Nc2ncc3c(Nc4cc(C(=O)NCCN5CCCC5(C)C)cnc4C)nn(C)c3n2)ccn1]

To a solution of 5-((6-chloro-1-methyl-1H-pyrazolo[3,4-d]pyrimidin-3-yl)amino)-N-(2-(2,2-dimethylpyrrolidin-1-yl)ethyl)-6-methylnicotinamide (400 mg, 0.9 mmol), N-(4-aminopyridin-2-yl)acetamide (205 mg, 1.35 mmol), potassium phosphate (575 mg, 2.71 mmol) and Brettphos (97 mg, 0.18 mmol) in DMF (8 mL) was added Brettphos-Pd-G3 (164 mg, 0.18 mmol) under N₂. The resulting mixture was stirred at 100° C. under N₂ for 12 h before cooled to 25° C. Then reaction mixture was concentrated under vacuum to give the crude product, which was purified by preparative high-performance liquid chromatography over column: Boston Uni C18 40*150*5 um and SFC over column:DAICEL CHIRALCEL OD-H (250 mm*30 mm, 5 um) to give the title compound 5-((6-((2-acetamidopyridin-4-yl)amino)-1-methyl-1H-pyrazolo[3,4-d]pyrimidin-3-yl)amino)-N-(2-(2,2-dimethylpyrrolidin-1-yl)ethyl)-6-methylnicotinamide as formate salt (37.7 mg, 7%) as a white solid. LCMS (ESI): mass calcd. for C₂₈H₃₅N₁₁O₂, 557.6; m/z found, 558.4 [M+H]⁺. ¹H NMR (400 MHz, DMSO-d₆) δ 10.34 (s, 1H), 10.30 (s, 1H), 9.27 (br s, 1H), 9.11 (s, 1H), 8.67 (d, J=1.8 Hz, 1H), 8.52-8.48 (m, 2H), 8.45 (br t, J=5.7 Hz, 1H), 8.08 (d, J=5.7 Hz, 1H), 7.27 (dd, J=1.9, 5.7 Hz, 1H), 3.93 (s, 3H), 3.32-3.28 (m, 4H), 2.77 (t, J=7.2 Hz, 2H), 2.62 (s, 3H), 2.11 (s, 3H), 1.74-1.63 (m, 2H), 1.59-1.51 (m, 2H), 0.93 (s, 6H).

### Example 101. N-(2-(2,2-dimethylpyrrolidin-1-yl)ethyl)-6-methyl-5-((1-methyl-6-((2-morpholinopyridin-4-yl)amino)-1H-pyrazolo[3,4-d]pyrimidin-3-yl)amino)nicotinamide

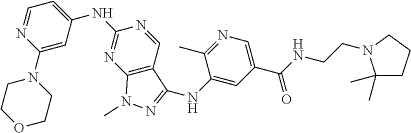 [SMILES: Cc1ncc(C(=O)NCCN2CCCC2(C)C)cc1Nc1nn(C)c2nc(Nc3ccnc(N4CCOCC4)c3)ncc12]

To a solution of 5-((6-chloro-1-methyl-1H-pyrazolo[3,4-d]pyrimidin-3-yl)amino)-N-(2-(2,2-dimethylpyrrolidin-1-yl)ethyl)-6-methylnicotinamide (80 mg, 0.18 mmol), 2-morpholinopyridin-4-amine (48.6 mg, 0.27 mmol) and Cs₂CO₃ (176 mg, 0.54 mmol) in DMF (8 mL) was added Pd₂(dba)₃ (33 mg, 0.04 mmol) and Brettphos (19.4 mg, 0.04 mmol) at room-temperature. The reaction mixture was stirred at 80° C. overnight before cooling to room-temperature. The mixture was concentrated under vacuum to give the crude product, which was purified by preparative high-performance liquid chromatography over Column: Phenomenex Gemini-NX C18 75*30 mm*3 um to give the title compound N-(2-(2,2-dimethylpyrrolidin-1-yl)ethyl)-6-methyl-5-((1-methyl-6-((2-morpholinopyridin-4-yl)amino)-1H-pyrazolo[3,4-d]pyrimidin-3-yl)amino)nicotinamide (26.2 mg, 22%) as a light yellow solid. LCMS (ESI): mass calcd. for C₃₀H₃₉N₁₁O₂, 585.703; m/z found, 586.20 [M+H]⁺. ¹H NMR (400 MHz, DMSO-d₆) δ 10.13 (s, 1H), 9.10 (s, 1H), 8.67 (s, 1H), 8.51 (d, J=6.68 Hz, 2H), 8.19 (s, 1H), 7.99 (d, J=5.60 Hz, 1H), 7.59 (s, 1H), 7.16 (d, J=5.60 Hz, 1H), 3.82 (s, 3H), 3.73 (br d, J=4.77 Hz, 4H), 3.42 (br d, J=4.41 Hz, 4H), 3.35-3.38 (m, 2H), 2.87-2.93 (m, 2H), 2.59-2.65 (m, 5H), 1.72 (br d, J=7.15 Hz, 2H), 1.61 (br d, J=7.99 Hz, 2H), 0.99 (s, 6H).

### Example 102. N-(2-(2,2-dimethylpyrrolidin-1-yl)ethyl)-6-methyl-5-((1-methyl-6-((6-morpholinopyridin-3-yl)amino)-1H-pyrazolo[3,4-d]pyrimidin-3-yl)amino)nicotinamide

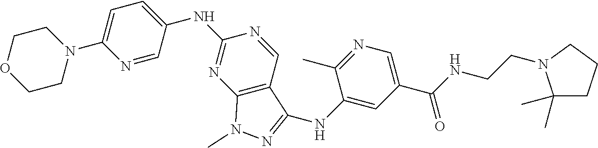 [SMILES: Cc1ncc(C(=O)NCCN2CCCC2(C)C)cc1Nc1nn(C)c2nc(Nc3ccc(N4CCOCC4)nc3)ncc12]

To a solution of 5-((6-chloro-1-methyl-1H-pyrazolo[3,4-d]pyrimidin-3-yl)amino)-N-(2-(2,2-dimethylpyrrolidin-1-yl)ethyl)-6-methylnicotinamide (180 mg, 0.41 mmol), 6-morpholinopyridin-3-amine (120 mg, 0.61 mmol) and Cs₂CO₃ (397 mg, 1.2 mmol) in DMF (9 mL) was added Pd₂(dba)₃ (75 mg, 0.08 mmol) and Brettphos (43.6 mg, 0.08 mmol) at room-temperature. The reaction mixture was stirred at 90° C. overnight before cooling to room-temperature. The mixture was concentrated under vacuum to give the crude product, which was purified by preparative high-performance liquid chromatography over Column: Phenomenex Gemini NX C18 150*40 mm*5 um to give the title compound N-(2-(2,2-dimethylpyrrolidin-1-yl)ethyl)-6-methyl-5-((1-methyl-6-((6-morpholinopyridin-3-yl)amino)-1H-pyrazolo[3,4-d]pyrimidin-3-yl)amino)nicotinamide (47.8 mg, 18%) as a brown solid. LCMS (ESI): mass calcd. for C₃₀H₃₉N₁₁O₂, 585.703; m/z found, 586.20 [M+H]⁺. ¹H NMR (400 MHz, DMSO-d₆) δ 9.64 (s, 1H), 8.94 (s, 1H), 8.54-8.64 (m, 3H), 8.44-8.47 (m, 1H), 8.39 (s, 1H), 8.00 (br d, J=7.50 Hz, 1H), 6.84 (d, J=9.26 Hz, 1H), 3.68-3.73 (m, 7H), 3.32-3.37 (m, 6H), 2.87 (br t, J=7.28 Hz, 2H), 2.56-2.64 (m, 5H), 1.66-1.74 (m, 2H), 1.55-1.60 (m, 2H), 0.96 (s, 6H).

### Example 103. N-(2-(2,2-dimethylpyrrolidin-1-yl)ethyl)-5-((6-((2-(4-(2-hydroxyethyl)piperazin-1-yl)pyridin-4-yl)amino)-1-methyl-1H-pyrazolo[3,4-d]pyrimidin-3-yl)amino)-6-methylnicotinamide

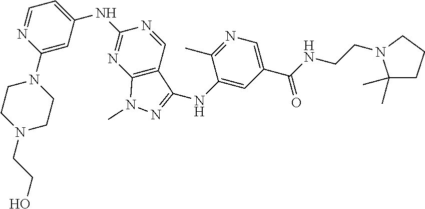 [SMILES: Cc1ncc(C(=O)NCCN2CCCC2(C)C)cc1Nc1nn(C)c2nc(Nc3ccnc(N4CCN(CCO)CC4)c3)ncc12]

### Step a: N-(2-(2,2-dimethylpyrrolidin-1-yl)ethyl)-5-((6-((2-(4-(2-hydroxyethyl)piperazin-1-yl)pyridin-4-yl)amino)-1-methyl-11H-pyrazolo[3,4-d]pyrimidin-3-yl)amino)-6-methylnicotinamide

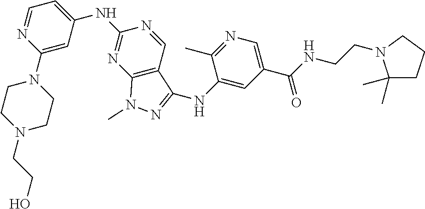 [SMILES: Cc1ncc(C(=O)NCCN2CCCC2(C)C)cc1Nc1nn(C)c2nc(Nc3ccnc(N4CCN(CCO)CC4)c3)ncc12]

To a solution of 5-((6-chloro-1-methyl-1H-pyrazolo[3,4-d]pyrimidin-3-yl)amino)-N-(2-(2,2-dimethylpyrrolidin-1-yl)ethyl)-6-methylnicotinamide (90 mg, 0.20 mmol), 2-(4-(4-aminopyridin-2-yl)piperazin-1-yl)ethanol (70 mg, 0.31 mmol) and Cs₂CO₃ (199 mg, 0.61 mmol) in DMF (9 mL) was added Brettphos pd G3 (37 mg, 0.04 mmol) and Brettphos (22 mg, 0.04 mmol) at room-temperature. The reaction mixture was stirred at 90° C. overnight before cooling to room-temperature. The mixture was concentrated under vacuum to give the crude product. The crude product was purified by preparative high-performance liquid chromatography over Column: Phenomenex Gemini NX C18 150*40 mm*5 um, then SFC over column: DAICEL CHIRALCEL OJ (250 mm*30 mm, 120 um) to give the title compound N-(2-(2,2-dimethylpyrrolidin-1-yl)ethyl)-5-((6-((2-(4-(2-hydroxyethyl)piperazin-1-yl)pyridin-4-yl)amino)-1-methyl-1H-pyrazolo[3,4-d]pyrimidin-3-yl)amino)-6-methylnicotinamide (48.5 mg, 23%) as a white solid. LCMS (ESI): mass calcd. for C₃₂H₄₄N₁₂O₂, 628.771; m/z found, 629.4 [M+H]⁺. ¹H NMR (400 MHz, METHANOL-d₄) δ 8.94 (s, 1H), 8.76 (d, J=1.98 Hz, 1H), 8.45 (d, J=1.98 Hz, 1H), 7.92 (d, J=5.95 Hz, 1H), 7.55 (s, 1H), 7.12 (dd, J=5.84, 1.43 Hz, 1H), 3.86 (s, 3H), 3.72 (t, J=5.95 Hz, 2H), 3.49-3.54 (m, 6H), 2.98 (br s, 2H), 2.74 (br s, 2H), 2.59-2.68 (m, 9H), 1.85 (br s, 2H), 1.72 (br d, J=8.38 Hz, 2H), 1.06 (s, 6H).

### Example 104.2-(4-((3-((5-((2-cyclohexylethyl)carbamoyl)-2-methylpyridin-3-yl)amino)-1-methyl-1H-pyrazolo[3,4-d]pyrimidin-6-yl)amino)phenyl)aceticacid

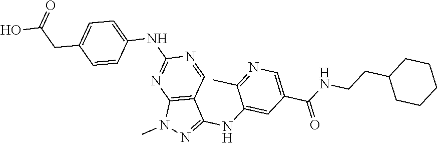 [SMILES: Cc1ncc(C(=O)NCCC2CCCCC2)cc1Nc1nn(C)c2nc(Nc3ccc(CC(=O)O)cc3)ncc12]

### Step a: 5-((6-chloro-1-methyl-1H-pyrazolo[3,4-d]pyrimidin-3-yl)amino)-N-(2-cyclohexylethyl)-6-methylnicotinamide

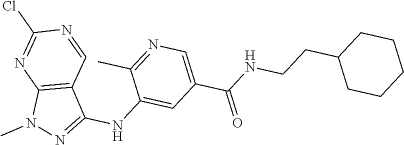 [SMILES: Cc1ncc(C(=O)NCCC2CCCCC2)cc1Nc1nn(C)c2nc(Cl)ncc12]

To a solution of ethyl 5-((6-chloro-1-methyl-1H-pyrazolo[3,4-d]pyrimidin-3-yl)amino)-6-methylnicotinate (200 mg, 0.487 mmol) in DCE (8 mL) was added trimethylaluminium (0.6 mL, 2M in toluene) and 2-cyclohexylethanamine (154 mg, 1.21 mmol) at 0° C. under N₂. The mixture was stirred at 60° C. for 5 h. The reaction mixture was quenched by aqueous HCl (4 mL, 1M), the mixture was concentrated to afford as black oil. MeOH (5 mL) and water (5 mL) added and the mixture stirred. The mixture was filtered and the filter cake was dried under vacuum to give 5-((6-chloro-1-methyl-1H-pyrazolo[3,4-d]pyrimidin-3-yl)amino)-N-(2-cyclohexylethyl)-6-methylnicotinamide (150 mg, 27%) as a yellow solid. LCMS (ESI): mass calcd. for C₂₁H₂₆C₁N₇O, 427.93; m/z found, 428.0 [M+H]⁺.

### Step b: tert-butyl 2-(4-((3-((5-((2-cyclohexylethyl)carbamoyl)-2-methylpyridin-3-yl)amino)-1-methyl-1H-pyrazolo[3,4-d]pyrimidin-6-yl)amino)phenyl)acetate

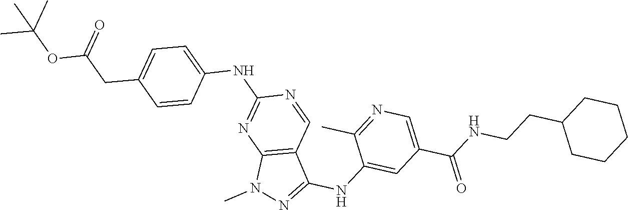 [SMILES: Cc1ncc(C(=O)NCCC2CCCCC2)cc1Nc1nn(C)c2nc(Nc3ccc(CC(=O)OC(C)(C)C)cc3)ncc12]

To a solution of 5-((6-chloro-1-methyl-1H-pyrazolo[3,4-d]pyrimidin-3-yl)amino)-N-(2-cyclohexylethyl)-6-methylnicotinamide (140 mg, 0.24 mmol), 2-tert-butyl 2-(4-aminophenyl)acetate (60.8 mg, 0.29 mmol) and Cesium Carbonate (238.8 mg, 0.73 mmol) in DMF (150 mL) was added Brettphos pd G3 (44.3 mg, 0.04 mmol) and Brettphos (26.3 mg, 0.05 mmol) at room temperature. The resultant mixture was stirred at 80° C. for 12 h. Then the mixture was concentrated under reduced pressure to give crude product, which was purified by column chromatography over silica gel (dichloromethanepetroleum/Methanol=8/2) to give the title compound tert-butyl 2-(4-((3-((5-((2-cyclohexylethyl)carbamoyl)-2-methylpyridin-3-yl)amino)-1-methyl-1H-pyrazolo[3,4-d]pyrimidin-6-yl)amino)phenyl)acetate (80 mg, 55%) as a yellow solid.

LCMS (ESI): mass calcd. for C₃₃H₄₂N₈O₃, 598.738; m/z found, 599.1 [M+H]⁺.

### Step f: 2-(4-((3-((5-((2-cyclohexylethyl)carbamoyl)-2-methylpyridin-3-yl)amino)-1-methyl-1H-pyrazolo[3,4-d]pyrimidin-6-yl)amino)phenyl)acetic acid

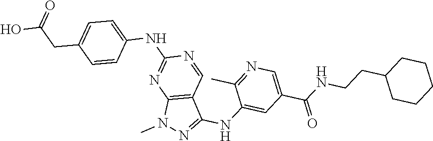 [SMILES: Cc1ncc(C(=O)NCCC2CCCCC2)cc1Nc1nn(C)c2nc(Nc3ccc(CC(=O)O)cc3)ncc12]

The solution of tert-butyl 2-(4-((3-((5-((2-cyclohexylethyl)carbamoyl)-2-methylpyridin-3-yl)amino)-1-methyl-1H-pyrazolo[3,4-d]pyrimidin-6-yl)amino)phenyl)acetate (80 mg, 0.13 mmol) in HCl solution (0.8 mL, 4M in dioxane) was stirred at 25° C. for 2 h. Then the reaction was concentrated under vacuum to give the crude product, which was purified by preparative high-performance liquid chromatography over Column: Welch Xtimate C18 150*30 mm*5 um to give the title compound 2-(4-((3-((5-((2-cyclohexylethyl)carbamoyl)-2-methylpyridin-3-yl)amino)-1-methyl-1H-pyrazolo[3,4-d]pyrimidin-6-yl)amino)phenyl)acetic acid (26.5 mg, 98%) as a white solid. LCMS (ESI): mass calcd. for C₂₉H₃₄N₈O₃, 542.632; m/z found, 543.2 [M+H]⁺. ¹H NMR (400 MHz, DMSO-d₆) δ 9.81 (s, 1H), 9.01 (s, 1H), 8.63 (d, J=1.8 Hz, 1H), 8.51-8.49 (m, 1H), 8.48-8.44 (m, 2H), 7.79 (br d, J=8.4 Hz, 2H), 7.20 (d, J=8.4 Hz, 2H), 3.77 (s, 3H), 3.45 (br s, 2H), 2.67-2.56 (m, 5H), 1.72-1.56 (m, 4H), 1.42 (q, J=6.9 Hz, 2H), 1.34-1.08 (m, 5H), 0.95-0.84 (m, 2H).

### Example 105. 5-((6-((6-(cyanomethyl)pyridin-3-yl)amino)-1-methyl-1H-pyrazolo[3,4-d]pyrimidin-3-yl)amino)-N-(2-cyclohexylethyl)-6-methylnicotinamide

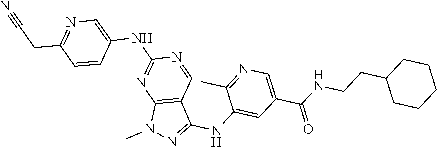 [SMILES: Cc1ncc(C(=O)NCCC2CCCCC2)cc1Nc1nn(C)c2nc(Nc3ccc(CC#N)nc3)ncc12]

To a solution of 5-((6-chloro-1-methyl-1H-pyrazolo[3,4-d]pyrimidin-3-yl)amino)-N-(2-cyclohexylethyl)-6-methylnicotinamide (100 mg, 0.18 mmol) in DMF (4 mL) was added 2-(5-aminopyridin-2-yl)acetonitrile (28 mg, 0.21 mmol) and Cs₂CO₃ (172 mg, 0.53 mmol), then Brettphos (19 mg, 0.04 mmol) and Brettphos-Pd-G3 (32 mg, 0.04 mmol) was added under N₂. The resulting mixture was charged with N₂ and heated at 80° C. and stirred for 14 hours. The mixture was cooled to room temperature and concentrated under vacuum to give the crude product, which was purified by preparative high-performance liquid chromatography over Column: Boston Prime C18 150*30 mm*5 um to give the title compound 5-((6-((6-(cyanomethyl)pyridin-3-yl)amino)-1-methyl-1H-pyrazolo[3,4-d]pyrimidin-3-yl)amino)-N-(2-cyclohexylethyl)-6-methylnicotinamide (25 mg, 25%) as a yellow solid. LCMS (ESI): mass calcd. for C₂₈H₃₂N₁₀O, 524.6; m/z found, 525.3 [M+H]⁺. 1H NMR (400 MHz, DMSO-d₆) δ 10.15 (s, 1H), 9.09-8.97 (m, 2H), 8.64 (s, 1H), 8.49 (br s, 3H), 8.37 (br d, J=7.3 Hz, 1H), 7.40 (d, J=8.5 Hz, 1H), 4.14 (s, 2H), 3.81 (s, 3H), 3.28 (br d, J=6.0 Hz, 2H), 2.61 (s, 3H), 1.76-1.62 (m, 5H), 1.48-1.38 (m, 2H), 1.31 (br s, 1H), 1.23-1.12 (m, 3H), 0.95-0.85 (m, 2H).

### Example 106. 2-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-2-yl)acetic acid

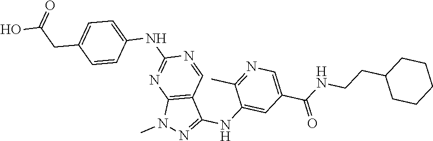 [SMILES: Cc1ncc(C(=O)NCCC2CCCCC2)cc1Nc1nn(C)c2nc(Nc3ccc(CC(=O)O)cc3)ncc12]

### Step a: ethyl 2-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-2-yl)acetate

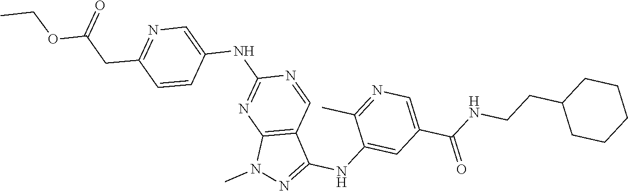 [SMILES: CCOC(=O)Cc1ccc(Nc2ncc3c(Nc4cc(C(=O)NCCC5CCCCC5)cnc4C)nn(C)c3n2)cn1]

To the mixture of 5-((6-chloro-1-methyl-1H-pyrazolo[3,4-d]pyrimidin-3-yl)amino)-N-(2-cyclohexylethyl)-6-methylnicotinamide (200 mg, 0.35 mmol) in DMF (4 mL) was added ethyl 2-(5-aminopyridin-2-yl)acetate (76 mg, 0.42 mmol) and Cs₂CO₃ (344 mg, 1.06 mmol), then Brettphos (38 mg, 0.07 mmol) and Brettphos-Pd-G3 (64 mg, 0.07 mmol) was added under N₂. The resulting mixture was charged with N₂ and heated at 80° C. and stirred for 16 hours. The mixture was cooled to room temperature and concentrated under vacuum to give the crude product, which was purified by silica gel chromatography (dichloromethane/Methanol from 100/0 to 80/20) to give the title compound ethyl 2-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-2-yl)acetate (100 mg, 50%) as a yellow solid. LCMS (ESI): mass calcd. for C₃₀H₃₇N₉O₃, 571.7; m/z found, 572.3 [M+H]⁺.

### Step b: 2-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-2-yl)acetic acid

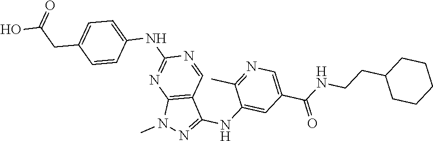 [SMILES: Cc1ncc(C(=O)NCCC2CCCCC2)cc1Nc1nn(C)c2nc(Nc3ccc(CC(=O)O)cc3)ncc12]

To a solution of ethyl 2-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-2-yl)acetate (10 mg, 0.18 mmol) in EtOH (3 mL) and water (1 mL) was added aqueous NaOH (0.48 mL, 0.95 mmol, 2 M) at room temperature. The reaction mixture was stirred at 25° C. for 1 hour. The reaction was concentrated under reduced pressure to give crude product, which was purified by preparative high-performance liquid chromatography over Column: Welch Xtimate C18 150*30 mm*5 um to give the title compound 2-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-2-yl)acetic acid (28 mg, 28%) as yellow solid. LCMS (ESI): mass calcd. for C₂₈H₃₃N₉O₃, 543.6; m/z found, 544.3 [M+H]⁺. ¹H NMR (400 MHz, DMSO-d₆) δ 10.01 (s, 1H), 9.05 (s, 1H), 8.92 (br s, 1H), 8.64 (s, 1H), 8.49 (s, 3H), 8.25 (br d, J=10.0 Hz, 1H), 7.30 (d, J=8.5 Hz, 1H), 3.79 (s, 3H), 3.65 (s, 2H), 3.28 (br d, J=6.3 Hz, 2H), 2.60 (s, 3H), 1.77-1.58 (m, 5H), 1.43 (q, J=7.0 Hz, 2H), 1.30 (br s, 1H), 1.25-1.10 (m, 3H), 0.95-0.83 (m, 2H).

### Example 107. 2-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-3-yl)acetic acid

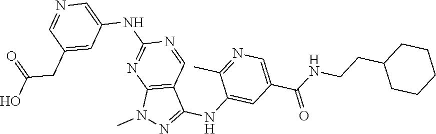 [SMILES: Cc1ncc(C(=O)NCCC2CCCCC2)cc1Nc1nn(C)c2nc(Nc3cncc(CC(=O)O)c3)ncc12]

### Step a: methyl 2-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-3-yl)acetate

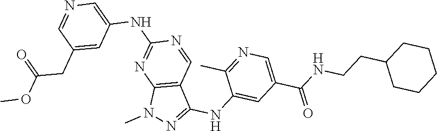 [SMILES: COC(=O)Cc1cncc(Nc2ncc3c(Nc4cc(C(=O)NCCC5CCCCC5)cnc4C)nn(C)c3n2)c1]

To a solution of 5-((6-chloro-1-methyl-1H-pyrazolo[3,4-d]pyrimidin-3-yl)amino)-N-(2-cyclohexylethyl)-6-methylnicotinamide (120 mg, 0.17 mmol) dissolved in DMF (10 mL) was added methyl 2-(5-aminopyridin-3-yl)acetate (33.7 mg 0.17 mmol), Then Cs₂CO₃ (162 mg 0.50 mmol), Brettphos (17.8 mg 0.03 mmol) and Brettphos-Pd-G3 (30.1 mg 0.03 mmol) was added. The mixture was charged with N₂. The mixture was stirred at 80° C. for 12 hours. The mixture was cooled to room temperature and filtered. The filtrate was concentrated to afford crude compound. The crude compound was purified by preparative high-performance liquid chromatography over Column: Phenomenex Gemini NX C18 150*40 mm*5 um to give the title compound methyl 2-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-3-yl)acetate (30 mg, 27%) as white powder. LCMS (ESI): mass calcd. for C₂₉H₃₅N₉O₃, 557.647; m/z found, 558.4 [M+H]⁺.

### Step b: 2-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-3-yl)acetic acid

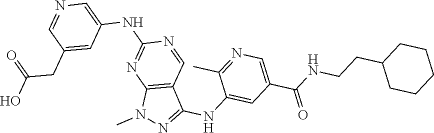 [SMILES: Cc1ncc(C(=O)NCCC2CCCCC2)cc1Nc1nn(C)c2nc(Nc3cncc(CC(=O)O)c3)ncc12]

To a solution of methyl 2-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-3-yl)acetate (25 mg, 0.04 mmol) in THF (0.6 mL) and H₂O (0.2 mL) was added LiOH (7.9 mg, 0.19 mmol). The mixture stirred at room temperature for 1.5 h. The reaction mixture was cooled to 0° C. and adjusted to pH=5 with aqueous HCl (1M). The resulting viscous oil was concentrated in vacuo to give the crude product, which was purified by preparative high-performance liquid chromatography over Column: Phenomenex Gemini-NX C18 75*30 mm*3 um to give the title compound 2-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-3-yl)acetic acid (9 mg, 16%) as a white solid. LCMS (ESI): mass calcd. for C₂₈H₃₃N₉O₃, 543.62; m/z found, 544.3 [M+H]⁺. ¹H NMR (400 MHz, DMSO-d₆) δ 10.20 (s, 1H), 9.12 (s, 1H), 8.93 (br s, 1H), 8.70 (s, 1H), 8.60 (s, 1H), 8.50-8.55 (m, 2H), 8.46 (s, 1H), 8.14 (s, 1H), 7.96 (s, 1H), 3.82 (s, 3H), 3.70 (s, 2H), 2.90 (s, 2H), 2.74 (s, 3H), 1.59-1.71 (m, 5H), 1.41-1.47 (m, 2H), 1.17-1.27 (m, 4H), 0.90 (br d, J=11.44 Hz, 2H).

### Example 108. N-(2-cyclohexylethyl)-5-((6-((2-cyclohexylethyl)amino)-1-methyl-1H-pyrazolo[3,4-d]pyrimidin-3-yl)amino)-6-methylnicotinamide

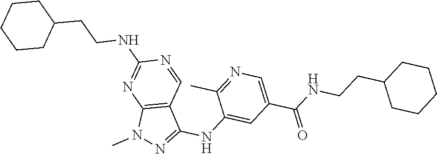 [SMILES: Cc1ncc(C(=O)NCCC2CCCCC2)cc1Nc1nn(C)c2nc(NCCC3CCCCC3)ncc12]

### Step a: N-(2-cyclohexylethyl)-5-((6-((2-cyclohexylethyl)amino)-1-methyl-1H-pyrazolo[3,4-d]pyrimidin-3-yl)amino)-6-methylnicotinamide

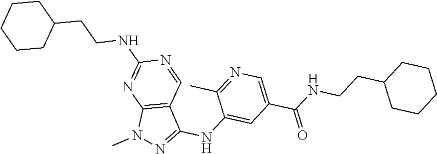 [SMILES: Cc1ncc(C(=O)NCCC2CCCCC2)cc1Nc1nn(C)c2nc(NCCC3CCCCC3)ncc12]

To a solution of ethyl 5-((6-chloro-1-methyl-1H-pyrazolo[3,4-d]pyrimidin-3-yl)amino)-6-methylnicotinate (500 mg, 1.3 mmol), 2-cyclohexylethanamine (418 mg, 3.3 mmol) and TMA (1.6 mL, 3.3 mmol) in DCE (8 mL). The mixture stirred at 60° C. for 8 h. and then aqueous HCl (4 mL, 1M) was added slowly. The mixture was concentrated under vacuum to give the crude product, which was purified by preparative high-performance liquid chromatography over Column: Boston prime C18 150*30 mm*5 um to give the title compound N-(2-cyclohexylethyl)-5-((6-((2-cyclohexylethyl)amino)-1-methyl-1H-pyrazolo[3,4-d]pyrimidin-3-yl)amino)-6-methylnicotinamide (30 mg, 4%) as a yellow solid. LCMS (ESI): mass calcd. for C₂₉H₄₂N₈O, 518.697; m/z found, 519.3 [M+H]⁺. ¹H NMR (400 MHz, METHANOL-d₄) δ 8.78 (s, 1H), 8.75-8.82 (m, 1H), 8.73 (d, J=1.91 Hz, 1H), 8.44 (d, J=1.79 Hz, 1H), 3.77 (s, 3H), 3.50 (t, J=7.21 Hz, 2H), 3.43 (t, J=7.39 Hz, 2H), 2.65 (s, 3H), 1.65-1.90 (m, 10H), 1.55 (dq, J=10.70, 7.08 Hz, 4H), 1.33-1.47 (m, 3H), 1.21-1.33 (m, 5H), 0.89-1.07 (m, 4H).

### Example 109. N-(2-cyclohexylethyl)-6-methyl-5-((1-methyl-6-((6-(piperazin-1-yl)pyridin-3-yl)amino)-1H-pyrazolo[3,4-d]pyrimidin-3-yl)amino)nicotinamide

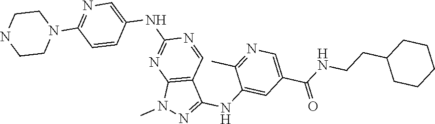 [SMILES: Cc1ncc(C(=O)NCCC2CCCCC2)cc1Nc1nn(C)c2nc(Nc3ccc(N4CCNCC4)nc3)ncc12]

### Step a: tert-butyl 4-(5-((3-((5-(ethoxycarbonyl)-2-methylpyridin-3-yl)amino)-1-methyl-1H-pyrazolo[3,4-d]pyrimidin-6-yl)amino)pyridin-2-yl)piperazine-1-carboxylate

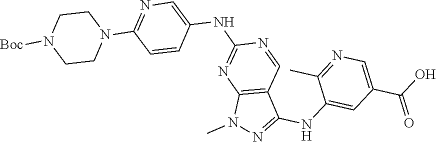 [SMILES: Cc1ncc(C(=O)O)cc1Nc1nn(C)c2nc(Nc3ccc(N4CCN(C(=O)OC(C)(C)C)CC4)nc3)ncc12]

To a solution of ethyl 5-((6-chloro-1-methyl-1H-pyrazolo[3,4-d]pyrimidin-3-yl)amino)-6-methylnicotinate (250 mg, 0.656 mmol), tert-butyl 4-(5-aminopyridin-2-yl)piperazine-1-carboxylate (219 mg, 0.788 mmol) and Cs₂CO₃ (640 mg, 1.96 mmol) in N,N-dimethylformamide (10 mL) was added Brettphos-Pd-G₃ (119 mg, 0.131 mmol) and Brettphos (70 mg, 0.131 mmol). The mixture was stirred at 80° C. for 16 h and then concentrated under vacuum to give the title compound tert-butyl 4-(5-((3-((5-(ethoxycarbonyl)-2-methylpyridin-3-yl)amino)-1-methyl-1H-pyrazolo[3,4-d]pyrimidin-6-yl)amino)pyridin-2-yl)piperazine-1-carboxylate (250 mg, 93%) as a black solid, which was used to next step without further purification. LCMS (ESI): mass calcd. for C₂₉H₃₆N₁₀O₄, 588.661; m/z found, 589.4 [M+H]⁺.

### Step b: 5-((6-((6-(4-(tert-butoxycarbonyl)piperazin-1-yl)pyridin-3-yl)amino)-1-methyl-1H-pyrazolo[3,4-d]pyrimidin-3-yl)amino)-6-methylnicotinic acid

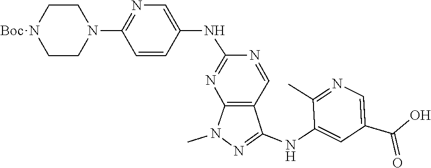 [SMILES: Cc1ncc(C(=O)O)cc1Nc1nn(C)c2nc(Nc3ccc(N4CCN(C(=O)OC(C)(C)C)CC4)nc3)ncc12]

To a solution of lithium hydrate (20 mg, 0.86 mmol) in methanol/THF/H₂O=1:3:1 (10 mL) was added tert-butyl 4-(5-((3-((5-(ethoxycarbonyl)-2-methylpyridin-3-yl)amino)-1-methyl-1H-pyrazolo[3,4-d]pyrimidin-6-yl)amino)pyridin-2-yl)piperazine-1-carboxylate (250 mg, 0.425 mmol) at room-temperature. The reaction mixture was stirred at room-temperature for 1 h. The mixture was adjust pH=3-4 with aqueous HCl (2 M). The mixture was filtered and washed with water (20 mL×3). The solid was evaporated under vacuum to give desired product 5-((6-((6-(4-(tert-butoxycarbonyl)piperazin-1-yl)pyridin-3-yl)amino)-1-methyl-1H-pyrazolo[3,4-d]pyrimidin-3-yl)amino)-6-methylnicotinic acid (200 mg, 59%) as black solid. LCMS (ESI): mass calcd. for C₂₇H₃₂N₁₀O₄, 560.6; m/z found, 561.3 [M+H]⁺.

### Step c: tert-butyl 4-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-2-yl)piperazine-1-carboxylate

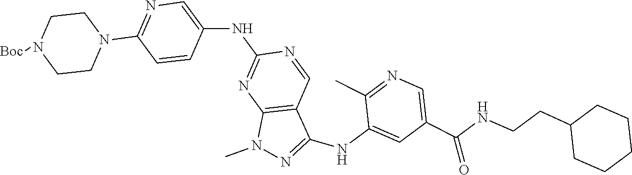 [SMILES: Cc1ncc(C(=O)NCCC2CCCCC2)cc1Nc1nn(C)c2nc(Nc3ccc(N4CCN(C(=O)OC(C)(C)C)CC4)nc3)ncc12]

To a solution of 5-((6-((6-(4-(tert-butoxycarbonyl)piperazin-1-yl)pyridin-3-yl)amino)-1-methyl-1H-pyrazolo[3,4-d]pyrimidin-3-yl)amino)-6-methylnicotinic acid (140 mg, 0.25 mmol), HATU (191 mg, 0.503 mmol) and N,N-diisopropylethylamine (129 mg, 1.005 mmol) in N,N-dimethylformamide (2 mL) was added 2-cyclohexylethanamine (32 mg, 0.25 mmol). The mixture stirred at 25° C. for 2 h and then concentrated under vacuum to give the crude product, which was purified by preparative high-performance liquid chromatography over Column: Boston prime C18 150*30 mm*5 um to give the title compound tert-butyl 4-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-2-yl)piperazine-1-carboxylate (30 mg, 18%) as a white solid. LCMS (ESI): mass calcd. for C₃₅H₄₇N₁₁O₃, 669.82; m/z found, 670.4 [M+H]⁺.

### Step d: N-(2-cyclohexylethyl)-6-methyl-5-((1-methyl-6-((6-(piperazin-1-yl)pyridin-3-yl)amino)-1H-pyrazolo[3,4-d]pyrimidin-3-yl)amino)nicotinamide

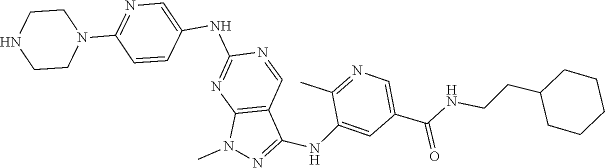 [SMILES: Cc1ncc(C(=O)NCCC2CCCCC2)cc1Nc1nn(C)c2nc(Nc3ccc(N4CCNCC4)nc3)ncc12]

To a solution of tert-butyl 4-(5-((3-((5-((2-cyclohexylethyl)carbamoyl)-2-methylpyridin-3-yl)amino)-1-methyl-1H-pyrazolo[3,4-d]pyrimidin-6-yl)amino)pyridin-2-yl)piperazine-1-carboxylate (30 mg, 0.04 mmol) in DCM (2 mL) was added HCl solution in MeOH (6 mL, 4M) at room-temperature. The reaction mixture was stirred at room-temperature for 1 h. The mixture was adjusted to pH=3-4 with aqueous HCl (2 M). The mixture was filtered and washed with water (20 mL×3). The solid was evaporated under vacuum to give desired product N-(2-cyclohexylethyl)-6-methyl-5-((1-methyl-6-((6-(piperazin-1-yl)pyridin-3-yl)amino)-1H-pyrazolo[3,4-d]pyrimidin-3-yl)amino)nicotinamide (20 mg, 73%) as gray solid. LCMS (ESI): mass calcd. for C₃₀H₃₉N₁₁O, 569.704; m/z found, 570.4 [M+H]⁺. ¹H NMR (400 MHz, DMSO-d₆) δ 9.76 (br s, 1H), 9.13 (br s, 2H), 9.03 (s, 1H), 8.68 (br s, 2H), 8.44-8.62 (m, 3H), 8.09 (br d, J=9.30 Hz, 1H), 6.98 (br d, J=9.06 Hz, 1H), 3.76 (s, 3H), 3.68 (br s, 4H), 3.27 (br s, 2H), 3.19 (br s, 4H), 2.63 (s, 3H), 2.00 (br d, J=7.75 Hz, 1H), 1.62-1.73 (m, 4H), 1.43 (q, J=7.03 Hz, 2H), 1.24 (br s, 2H), 1.17 (br d, J=10.37 Hz, 2H), 0.86-0.95 (m, 2H).

### Example 110. N-(2-cyclohexylethyl)-5-((6-((2-(4-(2-hydroxyethyl)piperazin-1-yl)pyridin-4-yl)amino)-1-methyl-1H-pyrazolo[3,4-d]pyrimidin-3-yl)amino)-6-methylnicotinamide

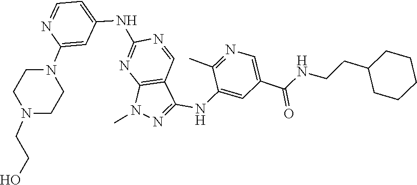 [SMILES: Cc1ncc(C(=O)NCCC2CCCCC2)cc1Nc1nn(C)c2nc(Nc3ccnc(N4CCN(CCO)CC4)c3)ncc12]

### Step d: ethyl 5-((6-((2-(4-(2-hydroxyethyl)piperazin-1-yl)pyridin-4-yl)amino)-1-methyl-1H-pyrazolo[3,4-d]pyrimidin-3-yl)amino)-6-methylnicotinate

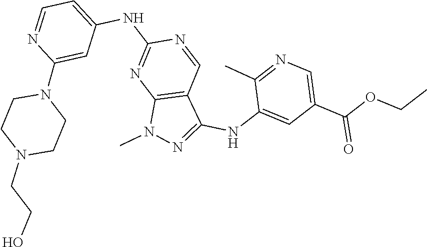 [SMILES: CCOC(=O)c1cnc(C)c(Nc2nn(C)c3nc(Nc4ccnc(N5CCN(CCO)CC5)c4)ncc23)c1]

To a solution of 5-((6-chloro-1-methyl-1H-pyrazolo[3,4-d]pyrimidin-3-yl)amino)-N-(2-cyclohexylethyl)-6-methylnicotinamide (300 mg, 0.79 mmol), 2-(4-(4-aminopyridin-2-yl)piperazin-1-yl)ethanol (217 mg, 0.95 mmol) and Cesium Carbonate (769 mg, 2.4 mmol) in DMF (10 mL) was added Brettphos pd G3 (143 mg, 0.16 mmol) and Brettphos (84.6 mg, 0.16 mmol) at room temperature. The resultant mixture was stirred at 80° C. for 12 h. Then the mixture was concentrated under reduced pressure to give crude product, which was purified by column chromatography over silica gel (dichloromethanepetroleum/Methanol=6:4) to give the title compound ethyl 5-((6-((2-(4-(2-hydroxyethyl)piperazin-1-yl)pyridin-4-yl)amino)-1-methyl-1H-pyrazolo[3,4-d]pyrimidin-3-yl)amino)-6-methylnicotinate (180 mg, 43%) as a yellow solid. LCMS (ESI): mass calcd. for C₂₆H₃₂N₁₀O₃, 532.597; m/z found, 533.3 [M+H]⁺.

### Step e: 5-((6-((2-(4-(2-hydroxyethyl)piperazin-1-yl)pyridin-4-yl)amino)-1-methyl-1H-pyrazolo[3,4-d]pyrimidin-3-yl)amino)-6-methylnicotinic acid

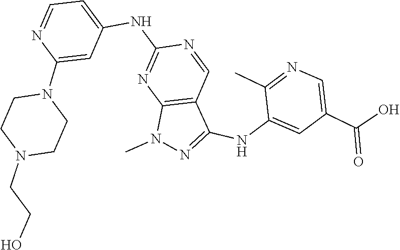 [SMILES: Cc1ncc(C(=O)O)cc1Nc1nn(C)c2nc(Nc3ccnc(N4CCN(CCO)CC4)c3)ncc12]

To a solution of ethyl 5-((6-((2-(4-(2-hydroxyethyl)piperazin-1-yl)pyridin-4-yl)amino)-1-methyl-1H-pyrazolo[3,4-d]pyrimidin-3-yl)amino)-6-methylnicotinate (150 mg, 0.28 mmol) in EtOH (1 mL) was added aqueous sodium hydroxide (0.29 mL, 0.57 mmol, 2M) at room temperature. The reaction mixture was stirred at 20° C. for 1 h. The mixture was adjusted to pH=3-4 with aqueous HCl (2 M). The mixture was filtered and washed with water (20 mL×3). The solid was evaporated under vacuum to give the desired product 5-((6-((2-(4-(2-hydroxyethyl)piperazin-1-yl)pyridin-4-yl)amino)-1-methyl-1H-pyrazolo[3,4-d]pyrimidin-3-yl)amino)-6-methylnicotinic acid (0.8 g, 94%) as black solid. LCMS (ESI): mass calcd. for C₂₄H₂₈N₁O₀₃, 504.544; m/z found, 505.3 [M+H]⁺.

### Step f: N-(2-cyclohexylethyl)-5-((6-((2-(4-(2-hydroxyethyl)piperazin-1-yl)pyridin-4-yl)amino)-1-methyl-1H-pyrazolo[3,4-d]pyrimidin-3-yl)amino)-6-methylnicotinamide

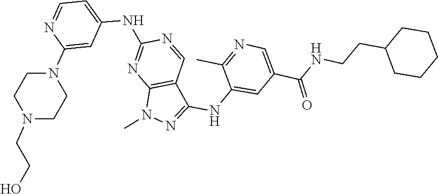 [SMILES: Cc1ncc(C(=O)NCCC2CCCCC2)cc1Nc1nn(C)c2nc(Nc3ccnc(N4CCN(CCO)CC4)c3)ncc12]

To a solution of 5-((6-((2-(4-(2-hydroxyethyl)piperazin-1-yl)pyridin-4-yl)amino)-1-methyl-1H-pyrazolo[3,4-d]pyrimidin-3-yl)amino)-6-methylnicotinic acid (100 mg, 0.20 mmol), HATU (90.4 mg, 0.24 mmol) and N,N-diisopropylethylamine (76.8 mg, 0.60 mmol) in N,N-dimethylformamide (5 mL) was added 2-cyclohexylethanamine (27.7 mg, 0.22 mmol). The mixture stirred at 25° C. for 1 h. Then the reaction was concentrated under vacuum to give the crude product, which was purified by preparative high-performance liquid chromatography over column: Welch Xtimate C18 150*30 mm*5 um to give the title compound N-(2-cyclohexylethyl)-5-((6-((2-(4-(2-hydroxyethyl)piperazin-1-yl)pyridin-4-yl)amino)-1-methyl-1H-pyrazolo[3,4-d]pyrimidin-3-yl)amino)-6-methylnicotinamide (39.5 mg, 96%) as a white solid. LCMS (ESI): mass calcd. for C₃₂H₄₃N₁₁O₂, 613.756; m/z found, 614.4 [M+H]⁺. ¹H NMR (400 MHz, DMSO-d₆) δ 10.07 (s, 1H), 9.09 (s, 1H), 8.65 (s, 1H), 8.50 (br d, J=9.3 Hz, 3H), 7.95 (d, J=5.8 Hz, 1H), 7.60 (s, 1H), 7.09 (br d, J=5.8 Hz, 1H), 4.51-4.43 (m, 1H), 3.81 (s, 3H), 3.59-3.50 (m, 3H), 3.46 (br s, 5H), 3.33-3.23 (m, 4H), 2.60 (s, 3H), 2.44 (br t, J=6.0 Hz, 2H), 1.76-1.58 (m, 5H), 1.42 (q, J=6.9 Hz, 2H), 1.29 (br s, 1H), 1.21-1.07 (m, 3H), 0.89 (q, J=11.5 Hz, 2H).

### Example 111. N-(2-(2,2-dimethylpyrrolidin-1-yl)ethyl)-5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinamide

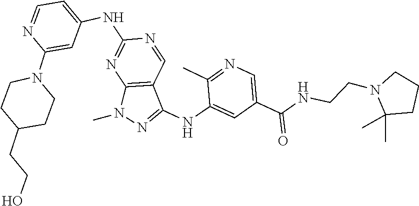 [SMILES: Cc1ncc(C(=O)NCCN2CCCC2(C)C)cc1Nc1nn(C)c2nc(Nc3ccnc(N4CCC(CCO)CC4)c3)ncc12]

### Step a: ethyl 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinate

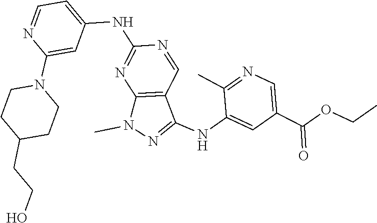 [SMILES: CCOC(=O)c1cnc(C)c(Nc2nn(C)c3nc(Nc4ccnc(N5CCC(CCO)CC5)c4)ncc23)c1]

To a solution of ethyl 5-((6-chloro-1-methyl-1H-pyrazolo[3,4-d]pyrimidin-3-yl)amino)-6-methylnicotinate (1.0 g, 2.1 mmol), 2-(1-(4-aminopyridin-2-yl)piperidin-4-yl)ethanol (0.4 g, 0.4 mmol) and Cesium Carbonate (2.4 g, 20.9 mmol) in THF (150 mL) was added Brettphos pd G3 (2.4 g, 20.9 mmol) and Brettphos (6.3 mL, 45.0 mmol) at room temperature. The resultant mixture was stirred at 80° C. for 12 h. Then the mixture was concentrated and triturated with tert-butyl ether (10 mL). The filter cake was dried in vacuum to give ethyl 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinate (0.9 g, 82%) as a red solid. LCMS (ESI): mass calcd. for C₂₇H₃₃N₉O₃, 531.609; m/z found, 532.6 [M+H]⁺.

### Step b: 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinic acid

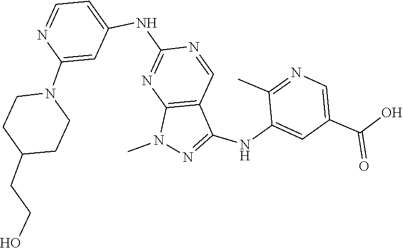 [SMILES: Cc1ncc(C(=O)O)cc1Nc1nn(C)c2nc(Nc3ccnc(N4CCC(CCO)CC4)c3)ncc12]

To a solution of ethyl 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinate (900 mg, 1.7 mmol) in EtOH (1.7 mL) was added aqueous sodium hydroxide (1.7 mL, 3.4 mmol, 2M) at room temperature. The reaction mixture was stirred at 20° C. for 1 h. The mixture was adjusted to pH=3-4 with aqueous HCl (2 M). The mixture was filtered and washed with water (20 mL×3). The solid was evaporated under vacuum to give the desired product 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinic acid (0.8 g, 94%) as white solid. LCMS (ESI): mass calcd. for C₂₅H₂₉N₉O₃, 503.556; m/z found, 504.3 [M+H]⁺.

### Step c: N-(2-(2,2-dimethylpyrrolidin-1-yl)ethyl)-5-((6-((2-(4-(2-hydroxyethyl)piperidin-1-yl)pyridin-4-yl)amino)-1-methyl-1H-pyrazolo[3,4-d]pyrimidin-3-yl)amino)-6 methylnicotinamide

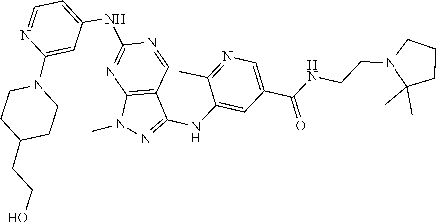 [SMILES: Cc1ncc(C(=O)NCCN2CCCC2(C)C)cc1Nc1nn(C)c2nc(Nc3ccnc(N4CCC(CCO)CC4)c3)ncc12]

To a solution of 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinic (100 mg, 0.20 mmol), HATU (90.6 mg, 0.24 mmol) and N,N-diisopropylethylamine (76.9 mg, 0.59 mmol) in N,N-dimethylformamide (5 mL) was added 2-(2,2-dimethylpyrrolidin-1-yl)ethanamine (31.1 mg, 0.22 mmol). The mixture stirred at 25° C. for 1 h and then concentrated under vacuum to give the crude product, which was purified by preparative high-performance liquid chromatography over Column: Welch Xtimate C18 150*30 mm*5 um to give the title compound N-(2-(2,2-dimethylpyrrolidin-1-yl)ethyl)-5-((6-((2-(4-(2-hydroxyethyl)piperidin-1-yl)pyridin-4-yl)amino)-1-methyl-1H-pyrazolo[3,4-d]pyrimidin-3-yl)amino)-6 methylnicotinamide (37.8 mg, 95%) as a white solid. LCMS (ESI): mass calcd. for C₃₃H₄₅N₁₁O₂, 627.783; m/z found, 628.5 [M+H]⁺. ¹H NMR (400 MHz, METHANOL-d₄) δ 8.91 (s, 1H), 8.80 (d, J=1.5 Hz, 1H), 8.42 (d, J=1.8 Hz, 1H), 7.86 (d, J=6.0 Hz, 1H), 7.57 (s, 1H), 7.00-6.93 (m, 1H), 4.16 (br d, J=13.0 Hz, 2H), 3.82 (s, 3H), 3.63 (t, J=6.6 Hz, 2H), 3.51 (t, J=6.9 Hz, 2H), 2.94 (br t, J=7.4 Hz, 2H), 2.83 (br t, J=11.8 Hz, 2H), 2.71 (br t, J=6.6 Hz, 2H), 2.61 (s, 3H), 1.85-1.75 (m, 4H), 1.73-1.64 (m, 3H), 1.49 (q, J=6.6 Hz, 2H), 1.32-1.17 (m, 2H), 1.05 (s, 6H).

### Example 112. N-(2-((2S,6S)-2,6-dimethylmorpholino)ethyl)-5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinamide

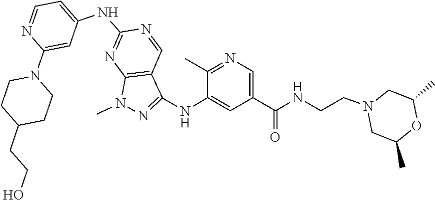 [SMILES: Cc1ncc(C(=O)NCCN2C[C@H](C)O[C@@H](C)C2)cc1Nc1nn(C)c2nc(Nc3ccnc(N4CCC(CCO)CC4)c3)ncc12]

### Step a: ethyl 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinate

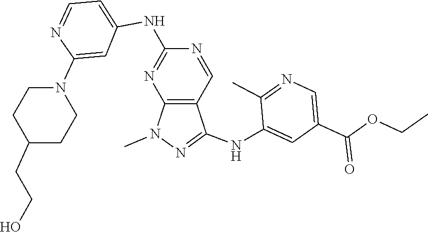 [SMILES: CCOC(=O)c1cnc(C)c(Nc2nn(C)c3nc(Nc4ccnc(N5CCC(CCO)CC5)c4)ncc23)c1]

To a solution of ethyl 5-((6-chloro-1-methyl-1H-pyrazolo[3,4-d]pyrimidin-3-yl)amino)-6-methylnicotinate (1.0 g, 2.1 mmol), 2-(1-(4-aminopyridin-2-yl)piperidin-4-yl)ethanol (0.4 g, 0.4 mmol) and Cesium Carbonate (2.4 g, 20.9 mmol) in THF (150 mL) was added Brettphos pd G3 (2.4 g, 20.9 mmol) and Brettphos (6.3 mL, 45.0 mmol) at room temperature. The resultant mixture was stirred at 80° C. for 12 h. Then the mixture was concentrated and triturated with Tert-butyl ether (10 mL). The filter cake was dried in vacuum to give ethyl 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinate (0.9 g, 82%) as a red solid. LCMS (ESI): mass calcd. for C₂₇H₃₃N₉O₃, 531.6; m/z found, 532.6 [M+H]⁺.

### Step b: 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinic acid

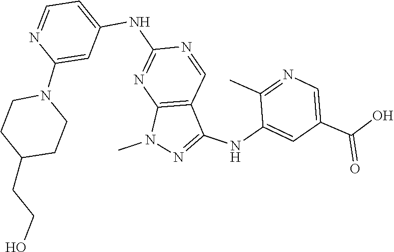 [SMILES: Cc1ncc(C(=O)O)cc1Nc1nn(C)c2nc(Nc3ccnc(N4CCC(CCO)CC4)c3)ncc12]

To a solution of ethyl 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinate (900 mg, 1.7 mmol) in EtOH (1.7 mL) was added aqueous sodium hydroxide (1.7 mL, 3.4 mmol, 2 M) at room temperature. The reaction mixture was stirred at 20° C. for 1 h. The mixture was adjusted to pH=3-4 with aqueous HCl (2 M). The mixture was filtered and washed with water (20 mL×3). The solid was evaporated under vacuum to give the desired product 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinic acid (0.8 g, 94%) as white solid. LCMS (ESI): mass calcd. for C₂₅H₂₉N₉O₃, 503.56; m/z found, 504.3 [M+H]⁺.

### Step c: N-(2-((2S,6S)-2,6-dimethylmorpholino)ethyl)-5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinamide

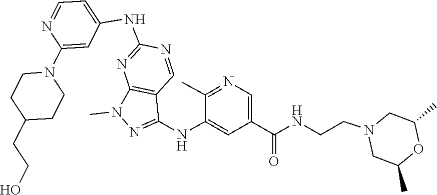 [SMILES: Cc1ncc(C(=O)NCCN2C[C@H](C)O[C@@H](C)C2)cc1Nc1nn(C)c2nc(Nc3ccnc(N4CCC(CCO)CC4)c3)ncc12]

To a solution of 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinic (150 mg, 0.29 mmol), HATU (136 mg, 0.36 mmol) and N,N-diisopropylethylamine (115 mg, 0.89 mmol) in N,N-dimethylformamide (5 mL) was added 2-((2S,6S)-2,6-dimethylmorpholino)ethanamine (51.9 mg, 0.33 mmol). The mixture stirred at 25° C. for 2 h and then concentrated under vacuum to give the crude product, which was purified by preparative high-performance liquid chromatography over Column: Welch Xtimate C18 150*30 mm*5 um to give the title compound N-(2-((2S,6S)-2,6-dimethylmorpholino)ethyl)-5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinamide (55.2 mg, 97%) as a white solid. LCMS (ESI): mass calcd. for C₃₃H₄₅N₁₁O₃, 643.78; m/z found, 644.4 [M+H]⁺. ¹H NMR (400 MHz, METHANOL-d₄) δ 8.95 (s, 1H), 8.79 (d, J=1.8 Hz, 1H), 8.42 (d, J=1.8 Hz, 1H), 7.89 (d, J=6.0 Hz, 1H), 7.62 (d, J=1.1 Hz, 1H), 7.02 (dd, J=1.7, 5.8 Hz, 1H), 4.20 (br d, J=12.8 Hz, 2H), 4.03-3.96 (m, 2H), 3.86 (s, 3H), 3.65 (t, J=6.6 Hz, 2H), 3.59-3.47 (m, 2H), 2.92-2.83 (m, 2H), 2.64 (s, 3H), 2.58-2.47 (m, 4H), 2.25 (dd, J=5.6, 10.9 Hz, 2H), 1.81 (br d, J=12.6 Hz, 2H), 1.77-1.67 (m, 1H), 1.52 (q, J=6.7 Hz, 2H), 1.33-1.25 (m, 2H), 1.20 (d, J=6.6 Hz, 6H).

### Example 113. (R)—N-(2-(2-ethylmorpholino)ethyl)-5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinamide

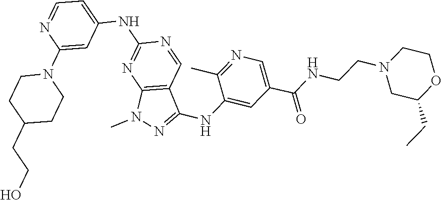 [SMILES: CC[C@@H]1CN(CCNC(=O)c2cnc(C)c(Nc3nn(C)c4nc(Nc5ccnc(N6CCC(CCO)CC6)c5)ncc34)c2)CCO1]

### Step a: ethyl 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinate

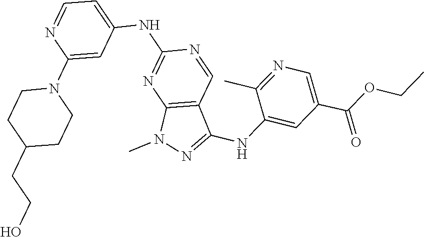 [SMILES: CCOC(=O)c1cnc(C)c(Nc2nn(C)c3nc(Nc4ccnc(N5CCC(CCO)CC5)c4)ncc23)c1]

To a solution of ethyl 5-((6-chloro-1-methyl-1H-pyrazolo[3,4-d]pyrimidin-3-yl)amino)-6-methylnicotinate (1.0 g, 2.1 mmol), 2-(1-(4-aminopyridin-2-yl)piperidin-4-yl)ethanol (0.4 g, 0.4 mmol) and Cesium Carbonate (2.4 g, 20.9 mmol) in THF (150 mL) was added Brettphos pd G3 (2.4 g, 20.9 mmol) and Brettphos (6.3 mL, 45.0 mmol) at room temperature. The resultant mixture was stirred at 80° C. for 12 h. Then the mixture was concentrated and triturated with Tert-butyl ether (10 mL). The filter cake was dried in vacuum to give ethyl 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinate (0.9 g, 82%) as a red solid. LCMS (ESI): mass calcd. for C₂₇H₃₃N₉O₃, 531.609; m/z found, 532.6 [M+H]⁺.

### Step b: 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinic acid

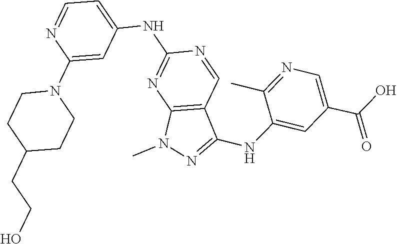 [SMILES: Cc1ncc(C(=O)O)cc1Nc1nn(C)c2nc(Nc3ccnc(N4CCC(CCO)CC4)c3)ncc12]

To a solution of ethyl 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinate (900 mg, 1.7 mmol) in EtOH (1.7 mL) was added aqueous sodium hydroxide (1.7 mL, 3.4 mmol, 2M) at room temperature. The reaction mixture was stirred at 20° C. for 1 h. The mixture was adjusted to pH=3-4 with aqueous HCl (2 M). The mixture was filtered and washed with water (20 mL×3). The solid was evaporated under vacuum to give the desired product 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinic acid (0.8 g, 94%) as white solid. LCMS (ESI): mass calcd. for C₂₅H₂₉N₉O₃, 503.556; m/z found, 504.3 [M+H]⁺.

### Step c: (R)—N-(2-(2-ethylmorpholino)ethyl)-5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinamide

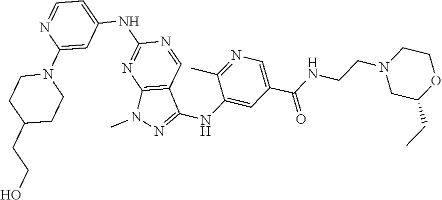 [SMILES: CC[C@@H]1CN(CCNC(=O)c2cnc(C)c(Nc3nn(C)c4nc(Nc5ccnc(N6CCC(CCO)CC6)c5)ncc34)c2)CCO1]

To a solution of 5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinic (100 mg, 0.19 mmol), HATU (90.6 mg, 0.24 mmol) and N,N-diisopropylethylamine (76.9 mg, 0.59 mmol) in N,N-dimethylformamide (5 mL) was added (R)-2-(2-ethylmorpholino)ethanamine (34.5 mg, 0.22 mmol). The mixture stirred at 25° C. for 2 h and then concentrated under vacuum to give the crude product, which was purified by preparative high-performance liquid chromatography over Column: Welch Xtimate C18 150*30 mm*5 um to give the title compound (R)—N-(2-(2-ethylmorpholino)ethyl)-5-((6-((2-(4-(2-hydroxyethyl)piperidin-1-yl)pyridin-4-yl)amino)-1-methyl-1H-pyrazolo[3,4-d]pyrimidin-3-yl)amino)-6-methylnicotinamide (31.1 mg, 96%) as a white solid. LCMS (ESI): mass calcd. for C₃₃H₄₅N₁₁O₃, 643.782; m/z found, 644.5 [M+H]⁺. ¹H NMR (400 MHz, METHANOL-d₄) δ 8.99 (s, 1H), 8.80 (d, J=2.01 Hz, 1H), 8.49 (d, J=2.01 Hz, 1H), 7.93 (d, J=5.77 Hz, 1H), 7.68 (d, J=1.25 Hz, 1H), 7.07 (dd, J=1.76, 5.77 Hz, 1H), 4.24 (br d, J=13.30 Hz, 2H), 3.80-3.96 (m, 4H), 3.64-3.72 (m, 3H), 3.51-3.62 (m, 2H), 3.37-3.46 (m, 1H), 2.81-2.99 (m, 4H), 2.68 (s, 3H), 2.63 (t, J=6.65 Hz, 2H), 2.21 (dt, J=3.39, 11.48 Hz, 1H), 1.82-1.97 (m, 3H), 1.75 (br s, 1H), 1.41-1.60 (m, 4H), 1.26-1.38 (m, 2H), 0.95 (t, J=7.53 Hz, 3H).

### Example 114. N-(2-((2S,6S)-2,6-dimethylmorpholino)ethyl)-6-methyl-5-((1-methyl-6-(pyrimidin-5-ylamino)-1H-pyrazolo[3,4-d]pyrimidin-3-yl)amino)nicotinamide

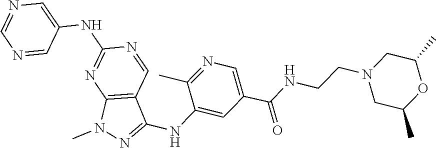 [SMILES: Cc1ncc(C(=O)NCCN2C[C@H](C)O[C@@H](C)C2)cc1Nc1nn(C)c2nc(Nc3cncnc3)ncc12]

### Step a: ethyl 6-methyl-5-((1-methyl-6-(pyrimidin-5-ylamino)-1H-pyrazolo[3,4-d]pyrimidin-3-yl)amino)nicotinate

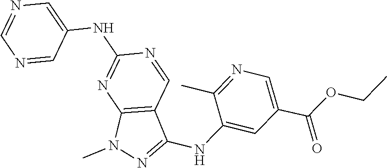 [SMILES: CCOC(=O)c1cnc(C)c(Nc2nn(C)c3nc(Nc4cncnc4)ncc23)c1]

To a solution of 5-((6-chloro-1-methyl-1H-pyrazolo[3,4-d]pyrimidin-3-yl)amino)-N-(2-cyclohexylethyl)-6-methylnicotinamide (550 mg, 1.1 mmol), pyrimidin-5-amine (129 mg, 1.4 mmol) and Cesium Carbonate (1.1 g, 3.4 mmol) in DMF (10 mL) was added Brettphos pd G3 (205 mg, 0.26 mmol) and Brettphos (121 mg, 0.23 mmol) at room temperature. The resultant mixture was stirred at 80° C. for 12 h. Then the mixture was concentrated and triturated with Tert-butyl ether (10 mL). The filter cake was dried in vacuum to give the title compound ethyl 6-methyl-5-((1-methyl-6-(pyrimidin-5-ylamino)-1H-pyrazolo[3,4-d]pyrimidin-3-yl)amino)nicotinate (350 mg, 76%) as a black solid. LCMS (ESI): mass calcd. for C₁₉H₁₉N₉O₂, 405.413; m/z found, 406.2 [M+H]⁺.

### Step b: 6-methyl-5-((1-methyl-6-(pyrimidin-5-ylamino)-1H-pyrazolo[3,4-d]pyrimidin-3-yl)amino)nicotinic acid

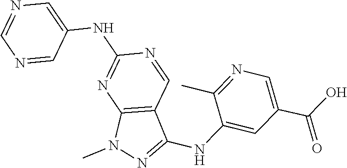 [SMILES: Cc1ncc(C(=O)O)cc1Nc1nn(C)c2nc(Nc3cncnc3)ncc12]

To a solution of ethyl 6-methyl-5-((1-methyl-6-(pyrimidin-5-ylamino)-1H-pyrazolo[3,4-d]pyrimidin-3-yl)amino)nicotinate (350 mg, 0.86 mmol) in EtOH (1 mL) was added aqueous sodium hydroxide (0.88 mL, 1.75 mmol, 2M) at room temperature. The reaction mixture was stirred at 20° C. for 1 h. The mixture was adjusted to pH=3-4 with aqueous HCl (2 M). The mixture was filtered and washed with water (20 mL×3). The solid was evaporated under vacuum to give the desired product 6-methyl-5-((1-methyl-6-(pyrimidin-5-ylamino)-1H-pyrazolo[3,4-d]pyrimidin-3-yl)amino)nicotinic acid (200 mg, 61%) as black solid. LCMS (ESI): mass calcd. for C_{P}H₁₅N₉O₂, 377.36 m/z found, 378.1 [M+H]⁺.

### Step c: N-(2-((2S,6S)-2,6-dimethylmorpholino)ethyl)-6-methyl-5-((1-methyl-6-(pyrimidin-5-ylamino)-1H-pyrazolo[3,4-d]pyrimidin-3-yl)amino)nicotinamide

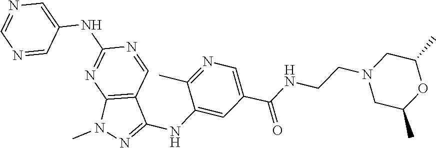 [SMILES: Cc1ncc(C(=O)NCCN2C[C@H](C)O[C@@H](C)C2)cc1Nc1nn(C)c2nc(Nc3cncnc3)ncc12]

To a solution of 6-methyl-5-((1-methyl-6-(pyrimidin-5-ylamino)-1H-pyrazolo[3,4-d]pyrimidin-3-yl)amino)nicotinic acid (100 mg, 0.27 mmol), HATU (121 mg, 0.32 mmol) and N,N-diisopropylethylamine (103 mg, 0.80 mmol) in N,N-dimethylformamide (4 mL) was added 2-((2S,6S)-2,6-dimethylmorpholino)ethanamine (46.1 mg, 0.29 mmol). The mixture stirred at 25° C. for 1 h. Then the reaction was concentrated under vacuum to give the crude product, which was purified by preparative high-performance liquid chromatography over column: Welch Xtimate C18 150*30 mm*5 um to give the title compound N-(2-((2S,6S)-2,6-dimethylmorpholino)ethyl)-6-methyl-5-((1-methyl-6-(pyrimidin-5-ylamino)-1H-pyrazolo[3,4-d]pyrimidin-3-yl)amino)nicotinamide (39.5 mg, 96%) as a white solid. LCMS (ESI): mass calcd. for C₂₅H₃₁N₁₁O₂, 517.586; m/z found, 518.4 [M+H]⁺. ¹H NMR (400 MHz, METHANOL-d₄) δ 9.33 (s, 2H), 9.01 (s, 1H), 8.79-8.76 (m, 2H), 8.45 (d, J=1.8 Hz, 1H), 4.03-3.96 (m, 2H), 3.88 (s, 3H), 3.59-3.48 (m, 2H), 2.66 (s, 3H), 2.60-2.49 (m, 4H), 2.25 (dd, J=5.6, 11.1 Hz, 2H), 1.20 (d, J=6.4 Hz, 6H).

### Example 115. N-(2-((2S,6S)-2,6-dimethylmorpholino)ethyl)-6-methyl-5-((1-methyl-6-(pyrimidin-5-ylamino)-1H-pyrazolo[3,4-d]pyrimidin-3-yl)amino)nicotinamide

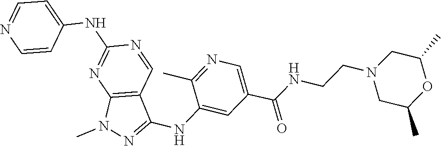 [SMILES: Cc1ncc(C(=O)NCCN2C[C@H](C)O[C@@H](C)C2)cc1Nc1nn(C)c2nc(Nc3ccncc3)ncc12]

### Step a: ethyl 6-methyl-5-((1-methyl-6-(pyridin-4-ylamino)-1H-pyrazolo[3,4-d]pyrimidin-3-yl)amino)nicotinate

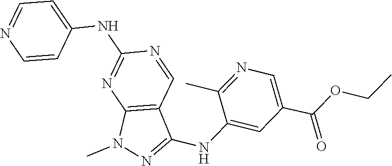 [SMILES: CCOC(=O)c1cnc(C)c(Nc2nn(C)c3nc(Nc4ccncc4)ncc23)c1]

To a solution of ethyl 5-((6-chloro-1-methyl-1H-pyrazolo[3,4-d]pyrimidin-3-yl)amino)-6-methylnicotinate (500 mg, 1.4 mmol), pyridin-4-amine (200 mg, 2.1 mmol) and Cesium Carbonate (1.4 g, 4.3 mmol) in DMF (10 mL) was added Brettphos pd G3 (261 mg, 0.29 mmol) and Brettphos (154 mg, 0.29 mmol) at room temperature. The resultant mixture was stirred at 80° C. for 12 h. Then the mixture was concentrated and triturated with tert-butyl ether (10 mL). The filter cake was dried in vacuum to give the title compound ethyl 6-methyl-5-((1-methyl-6-(pyridin-4-ylamino)-1H-pyrazolo[3,4-d]pyrimidin-3-yl)amino)nicotinate (350 mg, 60%) as a black solid. LCMS (ESI): mass calcd. for C₂₀H₂₀N₈O₂, 404.425; m/z found, 405.2 [M+H]⁺.

### Step e: 6-methyl-5-((1-methyl-6-(pyridin-4-ylamino)-1H-pyrazolo[3,4-d]pyrimidin-3-yl)amino)nicotinic acid

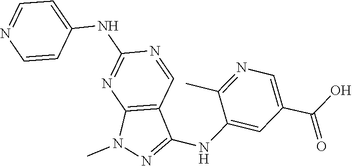 [SMILES: Cc1ncc(C(=O)O)cc1Nc1nn(C)c2nc(Nc3ccncc3)ncc12]

To a solution of ethyl 6-methyl-5-((1-methyl-6-(pyridin-4-ylamino)-1H-pyrazolo[3,4-d]pyrimidin-3-yl)amino)nicotinate (350 mg, 0.86 mmol) in EtOH (1 mL) was added aqueous sodium hydroxide (0.88 mL, 1.75 mmol, 2M) at room temperature. The reaction mixture was stirred at 20° C. for 1 h. The mixture was adjusted to pH=3-4 with aqueous HCl (2 M). The mixture was filtered and washed with water (20 mL×3). The solid was evaporated under vacuum to give the desired product 6-methyl-5-((1-methyl-6-(pyridin-4-ylamino)-1H-pyrazolo[3,4-d]pyrimidin-3-yl)amino)nicotinic acid (200 mg, 61%) as black solid. LCMS (ESI): mass calcd. for C₁₈H₁₆N₈O₂, 376.372 m/z found, 377.1 [M+H]⁺.

### Step f: N-(2-((2S, 6S)-2,6-dimethylmorpholino)ethyl)-6-methyl-5-((1-methyl-6-(pyridin-4-ylamino)-1H-pyrazolo[3,4-d]pyrimidin-3-yl)amino)nicotinamide

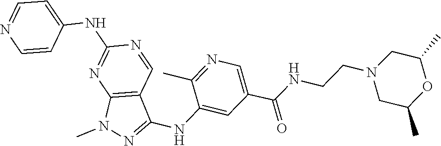 [SMILES: Cc1ncc(C(=O)NCCN2C[C@H](C)O[C@@H](C)C2)cc1Nc1nn(C)c2nc(Nc3ccncc3)ncc12]

To a solution of 6-methyl-5-((1-methyl-6-(pyridin-4-ylamino)-1H-pyrazolo[3,4-d]pyrimidin-3-yl)amino)nicotinic acid (150 mg, 0.40 mmol), HATU (182 mg, 0.48 mmol) and N,N-diisopropylethylamine (155 mg, 1.2 mmol) in N,N-dimethylformamide (1 mL) was added 2-((2S,6S)-2,6-dimethylmorpholino)ethanamine (69.4 mg, 0.44 mmol). The mixture stirred at 25° C. for 1 h. Then the reaction was concentrated under vacuum to give the crude product, which was purified by preparative high-performance liquid chromatography over column: Welch Xtimate C18 150*30 mm*5 um to give the title compound N-(2-((2S,6S)-2,6-dimethylmorpholino)ethyl)-6-methyl-5-((1-methyl-6-(pyridin-4-ylamino)-1H-pyrazolo[3,4-d]pyrimidin-3-yl)amino)nicotinamide (65.6 mg, 30%) as a white solid. LCMS (ESI): mass calcd. for C₂₆H₃₂N₁₀O₂, 516.598; m/z found, 517.3 [M+H]⁺. ¹H NMR (400 MHz, METHANOL-d₄) δ 8.98 (s, 1H), 8.77 (d, J=1.8 Hz, 1H), 8.42 (s, 1H), 8.32 (d, J=6.0 Hz, 2H), 7.91 (d, J=5.1 Hz, 2H), 4.01-3.93 (m, 2H), 3.88 (s, 3H), 3.57-3.45 (m, 2H), 2.63 (s, 3H), 2.58-2.44 (m, 4H), 2.23 (dd, J=5.6, 10.9 Hz, 2H), 1.18 (d, J=6.6 Hz, 6H).

### Example 116. N-(6-methyl-5-((1-methyl-6-((1-methyl-1H-pyrazol-4-yl)amino)-1H-pyrazolo[3,4-d]pyrimidin-3-yl)amino)pyridin-3-yl)-1-(4-methylpiperazine-1-carbonyl)cyclopropanecarboxamide

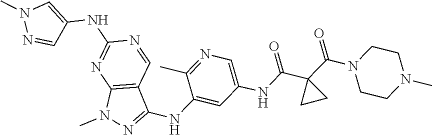

### Step a: ethyl 1-(4-methylpiperazine-1-carbonyl)cyclopropanecarboxylate

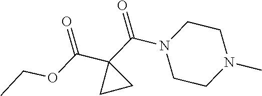 [SMILES: CCOC(=O)C1(C(=O)N2CCN(C)CC2)CC1]

To a solution of 1-(ethoxycarbonyl)cyclopropanecarboxylic acid (600 mg, 3.8 mmol) in DCM (12 mL) was added 1-methylpiperazine (570 mg, 5.7 mmol), 1H-benzo[d][1,2,3]triazol-1-ol (770 mg, 5.7 mmol), N¹-((ethylimino)methylene)-N³,N³-dimethylpropane-1,3-diamine hydrochloride (1.09 g, 5.7 mmol) and N-ethyl-N-isopropylpropan-2-amine (0.66 mL, 3.8 mmol) at room temperature. The reaction mixture was stirred at room temperature for 12 hours. The reaction mixture was quenched with water (15 mL). The mixture was extracted with DCM (3*40 mL). The organic layers were separated, dried with Na₂SO₄ and filtered. The filtrate was concentrated under reduced pressure to give crude product, which was purified by column chromatography over silica gel (eluent: petroleum ether:ethyl acetate=4:1) to give the title compound ethyl 1-(4-methylpiperazine-1-carbonyl)cyclopropanecarboxylate (0.95 g, 90%, crude) as a yellow oil.

¹H NMR (400 MHz, CDCl₃) δ 4.11 (q, J=7.1 Hz, 2H), 3.62 (br s, 2H), 3.53-3.45 (m, 2H), 2.36 (br s, 4H), 2.27 (s, 3H), 1.46-1.38 (m, 2H), 1.28-1.16 (m, 5H).

### Step b: 1-(4-methylpiperazine-1-carbonyl)cyclopropanecarboxylic acid

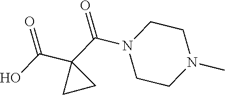

To a solution of ethyl 1-(4-methylpiperazine-1-carbonyl)cyclopropanecarboxylate (0.95 g, 4.0 mmol) in THF (20 mL) was added aqueous lithium hydroxide (7.9 mL, 1M in water, 7.9 mmol) at room-temperature. The resulting mixture was stirred at 30° C. for 12 hours. The mixture was adjust pH=4-5 with aqueous HCl (2 M). The mixture was concentrated under reduced pressure to give crude product, which was purified by preparative high-performance liquid chromatography over Column: Phenomenex Gemini NX C18 150*40 mm*5 um to give the title compound 1-(4-methylpiperazine-1-carbonyl)cyclopropanecarboxylic acid (0.95 g, 97%) as white solid. LCMS (ESI): mass calcd. for C₁₀H₁₆N₂O₃·HCl, 212.2; m/z found, 213.2 [M+H]⁺. ¹H NMR (400 MHz, DMSO-d₆) δ 3.68 (br s, 4H), 2.95 (br d, J=18.0 Hz, 4H), 2.61 (s, 3H), 1.35-1.29 (m, 2H), 1.25-1.19 (m, 2H).

### Step c: N-(6-methyl-5-((1-methyl-6-((1-methyl-1H-pyrazol-4-yl)amino)-1H-pyrazolo[3,4-d]pyrimidin-3-yl)amino)pyridin-3-yl)-1-(4-methylpiperazine-1-carbonyl)cyclopropanecarboxamide

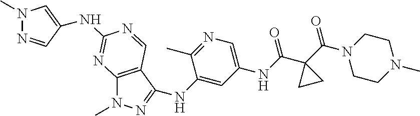

To a solution of 1-(4-methylpiperazine-1-carbonyl)cyclopropanecarboxylic acid (60 mg, 0.24 mmol), TCFH (135 mg, 0.48 mmol) and NMI (139 mg, 1.7 mmol) in N,N-dimethylformamide (8 mL) was added N³-(5-amino-2-methylpyridin-3-yl)-1-methyl-N⁶-(1-methyl-1H-pyrazol-4-yl)-1H-pyrazolo[3,4-d]pyrimidine-3,6-diamine (97 mg, 0.22 mmol). The mixture stirred at 25° C. for 2 h and then concentrated under vacuum to give the crude product, which was purified by preparative high-performance liquid chromatography over Column: Phenomenex Gemini-NX 80*40 mm*3 um to give the title compound N-(6-methyl-5-((1-methyl-6-((1-methyl-1H-pyrazol-4-yl)amino)-1H-pyrazolo[3,4-d]pyrimidin-3-yl)amino)pyridin-3-yl)-1-(4-methylpiperazine-1-carbonyl)cyclopropanecarboxamide (51 mg, 37%) as an off-white solid. LCMS (ESI): mass calcd. for C₂₆H₃₂N₁₂O₂, 544.6; m/z found, 545.3 [M+H]⁺. ¹H NMR (400 MHz, METHANOL-d₄) δ 8.84 (s, 1H), 8.57 (s, 1H), 8.28 (s, 1H), 8.10 (s, 1H), 7.67 (s, 1H), 3.92 (s, 3H), 3.85 (s, 3H), 3.69 (br s, 4H), 2.56 (s, 3H), 2.47 (br s, 4H), 2.30 (s, 3H), 1.57-1.49 (m, 2H), 1.40-1.32 (m, 2H).

### Example 117. N-(6-methyl-5-((1-methyl-6-((1-methyl-1H-pyrazol-4-yl)amino)-1H-pyrazolo[3,4-d]pyrimidin-3-yl)amino)pyridin-3-yl)-1-(piperazine-1-carbonyl) cyclopropanecarboxamide

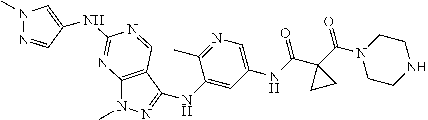 [SMILES: Cc1ncc(NC(=O)C2(C(=O)N3CCNCC3)CC2)cc1Nc1nn(C)c2nc(Nc3cnn(C)c3)ncc12]

### Step a: tert-butyl 4-(1-(ethoxycarbonyl)cyclopropanecarbonyl)piperazine-1-carboxylate

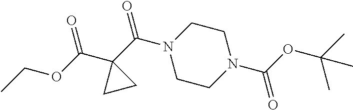 [SMILES: CCOC(=O)C1(C(=O)N2CCN(C(=O)OC(C)(C)C)CC2)CC1]

To a solution of 1-(ethoxycarbonyl)cyclopropanecarboxylic acid (500 mg, 3.2 mmol) in DCM (12 mL) was added tert-butyl piperazine-1-carboxylate (883 mg, 4.7 mmol), 1H-benzo[d][1,2,3]triazol-1-ol (641 mg, 4.7 mmol), N¹-((ethylimino)methylene)-N³,N³-dimethylpropane-1,3-diamine hydrochloride (909 mg, 4.7 mmol) and N-ethyl-N-isopropylpropan-2-amine (0.55 mL, 3.2 mmol) at room temperature. The reaction mixture was stirred at room temperature for 12 hours. The reaction mixture was quenched with water (15 mL). The mixture was extracted with DCM (3*40 mL). The organic layers were separated, dried with anhydrous Na₂SO₄ and filtered. The filtrate was concentrated under reduced pressure to give crude product, which was purified by column chromatography over silica gel (eluent: petroleum ether:ethyl acetate=4:1) to give the title compound tert-butyl 4-(1-(ethoxycarbonyl)cyclopropanecarbonyl)piperazine-1-carboxylate (1.1 g, 90%, crude) as a brown oil. ¹H NMR (400 MHz, CDCl₃) δ 4.11 (q, J=7.2 Hz, 1H), 4.06 (s, 1H), 3.54 (br d, J=5.2 Hz, 2H), 3.46-3.33 (m, 6H), 1.46-1.37 (m, 11H), 1.28-1.23 (m, 2H), 1.20 (t, J=7.1 Hz, 3H).

### Step b: 1-(4-methylpiperazine-1-carbonyl)cyclopropanecarboxylic acid

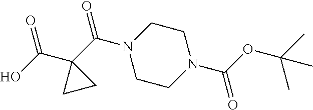 [SMILES: CC(C)(C)OC(=O)N1CCN(C(=O)C2(C(=O)O)CC2)CC1]

To a solution of tert-butyl 4-(1-(ethoxycarbonyl)cyclopropanecarbonyl)piperazine-1-carboxylate (1.05 g, 3.2 mmol) in THF (20 mL) was added aqueous lithium hydroxide (6.4 mL, 1M in water, 6.4 mmol) at room-temperature. The resulting mixture was stirred at 30° C. for 12 hours. The mixture was adjusted to pH=4-5 with aqueous HCl (2 M). The mixture filtered and the filter cake was washed with water (10 mL). The filter cake was dried under vacuum to give the title compound 1-(4-(tert-butoxycarbonyl)piperazine-1-carbonyl) cyclopropanecarboxylic acid (900 mg, 94%) as white solid. ¹H NMR (400 MHz, DMSO-d₆) δ 12.85 (br s, 1H), 3.44 (br s, 4H), 3.35 (br s, 4H), 1.41 (s, 9H), 1.33-1.27 (m, 2H), 1.22-1.17 (m, 2H).

### Step c: tert-butyl 4-(1-((6-methyl-5-((1-methyl-6-((1-methyl-1H-pyrazol-4-yl)amino)-1H-pyrazolo[3,4-d]pyrimidin-3-yl)amino)pyridin-3-yl)carbamoyl)cyclopropanecarbonyl) piperazine-1-carboxylate

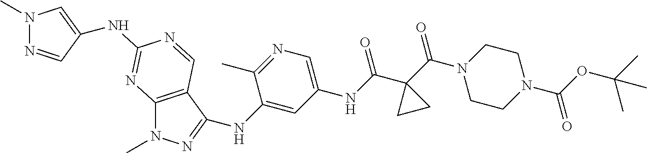 [SMILES: Cc1ncc(NC(=O)C2(C(=O)N3CCN(C(=O)OC(C)(C)C)CC3)CC2)cc1Nc1nn(C)c2nc(Nc3cnn(C)c3)ncc12]

To a solution of 1-(4-(tert-butoxycarbonyl)piperazine-1-carbonyl)cyclopropanecarboxylic acid (80 mg, 0.27 mmol), TCFH (150 mg, 0.54 mmol) and NMI (154 mg, 1.9 mmol) in N,N-dimethylformamide (8 mL) was added N³-(5-amino-2-methylpyridin-3-yl)-1-methyl-N⁶-(1-methyl-1H-pyrazol-4-yl)-1H-pyrazolo[3,4-d]pyrimidine-3,6-diamine (108 mg, 0.24 mmol). The mixture stirred at 25° C. for 2 h and then concentrated under vacuum to give the crude product, which was purified by flash column chromatography over a 4 g silica gel (DCM:CH₃OH=10:1) to give the title compound tert-butyl 4-(1-((6-methyl-5-((1-methyl-6-((1-methyl-1H-pyrazol-4-yl)amino)-1H-pyrazolo[3,4-d]pyrimidin-3-yl)amino)pyridin-3-yl)carbamoyl)cyclopropanecarbonyl piperazine-1-carboxylate (134 mg, 69%) as a pale brown solid. LCMS (ESI): mass calcd. for C₃₀H₃₈N₁₂O₄, 630.7; m/z found, 631.4 [M+H]⁺.

### Step d: N-(6-methyl-5-((1-methyl-6-((1-methyl-1H-pyrazol-4-yl)amino)-1H-pyrazolo[3,4-d]pyrimidin-3-yl)amino)pyridin-3-yl)-1-(piperazine-1-carbonyl)cyclopropanecarboxamide

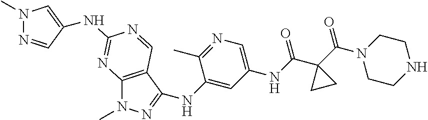 [SMILES: Cc1ncc(NC(=O)C2(C(=O)N3CCNCC3)CC2)cc1Nc1nn(C)c2nc(Nc3cnn(C)c3)ncc12]

A mixture of tert-butyl 4-(1-((6-methyl-5-((1-methyl-6-((1-methyl-1H-pyrazol-4-yl)amino)-1H-pyrazolo[3,4-d]pyrimidin-3-yl)amino)pyridin-3-yl)carbamoyl)cyclopropanecarbonyl piperazine-1-carboxylate (200 mg, 0.17 mmol) in HCl/MeOH (6 mL, 4 M, 24 mmol) was stirred at 25° C. for 2 hours. The mixture was adjusted to pH-7 with NH₃—H₂O and then concentrated under vacuum to give the crude product, which was purified by preparative high-performance liquid chromatography over Column: Phenomenex Gemini-NX 80*40 mm*3 um to give the title compound N-(6-methyl-5-((1-methyl-6-((1-methyl-1H-pyrazol-4-yl)amino)-1H-pyrazolo[3,4-d]pyrimidin-3-yl)amino)pyridin-3-yl)-1-(piperazine-1-carbonyl)cyclopropanecarboxamide (47 mg, 52%) as an off-white solid. LCMS (ESI): mass calcd. for C₂₅H₃₀N₁₂O₂, 530.6; m/z found, 531.3 [M+H]⁺. ¹H NMR (400 MHz, METHANOL-d₄) δ 8.84 (s, 1H), 8.56 (br s, 1H), 8.29 (br s, 1H), 8.10 (br s, 1H), 7.67 (s, 1H), 3.92 (s, 3H), 3.86 (s, 3H), 3.65 (br d, J=4.3 Hz, 4H), 2.89-2.81 (m, 4H), 2.56 (s, 3H), 1.56-1.50 (m, 2H), 1.39-1.34 (m, 2H).

### Example 118. N-(2-(4,4-difluoropiperidin-1-yl)ethyl)-4-methyl-3-((1-methyl-6-((1-methyl-1H-pyrazol-4-yl)amino)-1H-pyrazolo[3,4-d]pyrimidin-3-yl)amino)benzamide

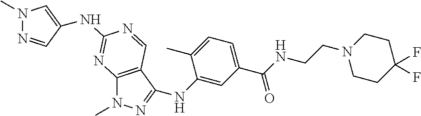 [SMILES: Cc1ccc(C(=O)NCCN2CCC(F)(F)CC2)cc1Nc1nn(C)c2nc(Nc3cnn(C)c3)ncc12]

### Step a: ethyl 3-(2,4-dichloropyrimidine-5-carboxamido)-4-methylbenzoate

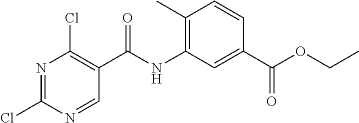 [SMILES: CCOC(=O)c1ccc(C)c(NC(=O)c2cnc(Cl)nc2Cl)c1]

To a solution of ethyl 5-amino-6-methylnicotinate (1.0 g, 4.7 mmol) in Dichloromethane (150 mL) was added ethyl 3-amino-4-methylbenzoate (0.76 g, 4.3 mmol). The mixture stirred at 25° C. for 12 h and then the reaction was concentrated under vacuum to give the crude product, which was purified by column chromatography over silica gel (eluent: dichloromethane/methyl alcohol=9:1) to give the title compound ethyl 3-(2,4-dichloropyrimidine-5-carboxamido)-4-methylbenzoate (900 mg, 54%) as a yellow solid. LCMS (ESI): mass calcd. for C₁₅H₁₃Cl₂N₃O₃, 353.03; m/z found, 354.0 [M+H]⁺.

### Step b: ethyl 3-(2-chloro-4-(1-methylhydrazinyl)pyrimidine-5-carboxamido)-4-methylbenzoate

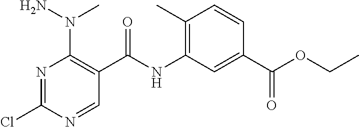 [SMILES: CCOC(=O)c1ccc(C)c(NC(=O)c2cnc(Cl)nc2N(C)N)c1]

To a solution of ethyl 3-(2,4-dichloropyrimidine-5-carboxamido)-4-methylbenzoate (0.80 g, 2.3 mmol) and TEA (0.79 mL, 5.6 mmol) in THF (200 mL) was added methylhydrazine (0.33 mL, 2.5 mmol, 40% in H₂O). The resulting mixture was quenched with water (300 mL) and extracted with ethyl acetate (300 mL×3). The combined organic phases were dried over anhydrous Na₂SO₄ and filtered. The filtrate was concentrated under reduced pressure to give the title compound ethyl 3-(2-chloro-4-(1-methylhydrazinyl)pyrimidine-5-carboxamido)-4-methylbenzoate (0.8 g, 62%) as a yellow solid. LCMS (ESI): mass calcd. for C₁₆H₁₈ClN₅O₃, 363.11; m/z found, 364.1 [M+H]⁺.

### Step c: ethyl 3-((6-chloro-1-methyl-1H-pyrazolo[3,4-d]pyrimidin-3-yl)amino)-4-methylbenzoate

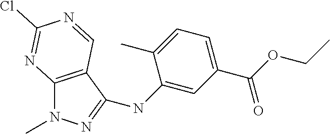 [SMILES: CCOC(=O)c1ccc(C)c(Nc2nn(C)c3nc(Cl)ncc23)c1]

To a solution of ethyl 3-(2-chloro-4-(1-methylhydrazinyl)pyrimidine-5-carboxamido)-4-methylbenzoate (0.80 g, 1.4 mmol) in toluene (60 mL) was added PCl₅(294 mg, 1.4 mmol). The mixture stirred at 120° C. for 12 h. Then the reaction was quenched with water (30 mL) and filtered. The filtrate was then concentrated to dryness under reduced pressure to give the title compound ethyl 3-((6-chloro-1-methyl-1H-pyrazolo[3,4-d]pyrimidin-3-yl)amino)-4-methylbenzoate (450 mg, 92%) as a yellow solid. LCMS (ESI): mass calcd. for C₁₆H₁₆ClN₅O₂, 345.784; m/z found, 346.7 [M+H]⁺.

### Step d: ethyl 4-methyl-3-((1-methyl-6-((1-methyl-1H-pyrazol-4-yl)amino)-1H-pyrazolo[3,4-d]pyrimidin-3-yl)amino)benzoate

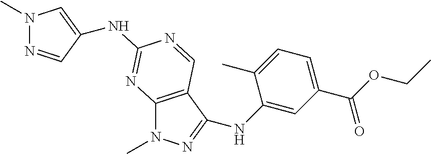 [SMILES: CCOC(=O)c1ccc(C)c(Nc2nn(C)c3nc(Nc4cnn(C)c4)ncc23)c1]

To a solution of ethyl 3-((6-chloro-1-methyl-1H-pyrazolo[3,4-d]pyrimidin-3-yl)amino)-4-methylbenzoate (450 mg, 1.3 mmol), 1-methyl-1H-pyrazol-4-amine (151 mg, 1.6 mmol) and Cs₂CO₃ (1.3 g, 3.9 mmol) in N,N-dimethylformamide (8 mL) was added Brettphos-Pd-G3 (236 mg, 0.26 mmol) and Brettphos (139 mg, 0.26 mmol). The mixture stirred at 80° C. for 3 h. Then the reaction was concentrated under vacuum to give the crude product, which was washed with MTBE (10 mL). The filtrate was then concentrated to dryness under reduced pressure to give the title compound ethyl 4-methyl-3-((1-methyl-6-((1-methyl-1H-pyrazol-4-yl)amino)-1H-pyrazolo[3,4-d]pyrimidin-3-yl)amino)benzoate (450 mg, 85%) as a black solid. LCMS (ESI): mass calcd. for C₂₀H₂₂N₈O₂, 406.19; m/z found, 407.2 [M+H]⁺.

### Step e: 4-methyl-3-((1-methyl-6-((1-methyl-1H-pyrazol-4-yl)amino)-1H-pyrazolo[3,4-d]pyrimidin-3-yl)amino)benzoic acid

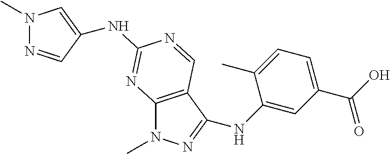 [SMILES: Cc1ccc(C(=O)O)cc1Nc1nn(C)c2nc(Nc3cnn(C)c3)ncc12]

To a solution of ethyl 4-methyl-3-((1-methyl-6-((1-methyl-1H-pyrazol-4-yl)amino)-1H-pyrazolo[3,4-d]pyrimidin-3-yl)amino)benzoate (450 mg, 1.1 mmol) in EtOH (3 mL) was added aqueous lithium hydroxide (3 mL, 6.0 mmol, 1M in water) at room temperature. The reaction mixture was stirred at 20° C. for 1 h. The mixture was adjusted to pH=3-4 with aqueous HCl (2 M). The mixture was filtered and washed with water (20 mL×3). The solid was evaporated under vacuum to give the desired product 4-methyl-3-((1-methyl-6-((1-methyl-1H-pyrazol-4-yl)amino)-1H-pyrazolo[3,4-d]pyrimidin-3-yl)amino)benzoic acid (350 mg, 84%) as black solid. LCMS (ESI): mass calcd. for C₁₈H₁₈N₈O₂, 378.16; m/z found, 379.0 [M+H]⁺.

### Step f: N-(2-(4,4-difluoropiperidin-1-yl)ethyl)-4-methyl-3-((1-methyl-6-((1-methyl-1H-pyrazol-4-yl)amino)-1H-pyrazolo[3,4-d]pyrimidin-3-yl)amino)benzamide

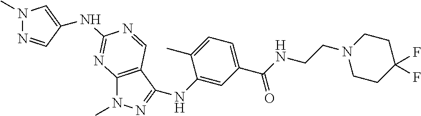 [SMILES: Cc1ccc(C(=O)NCCN2CCC(F)(F)CC2)cc1Nc1nn(C)c2nc(Nc3cnn(C)c3)ncc12]

To a solution of 4-methyl-3-((1-methyl-6-((1-methyl-1H-pyrazol-4-yl)amino)-1H-pyrazolo[3,4-d]pyrimidin-3-yl)amino)benzoic acid (100 mg, 0.26 mmol), HATU (120 mg, 0.32 mmol) and N,N-diisopropylethylamine (102 mg, 0.79 mmol) in N,N-dimethylformamide (5 mL) was added ethyl 3-amino-4-methylbenzoate (47.7 mg, 0.29 mmol). The mixture stirred at room temperature for 2 h. Then the reaction was concentrated under vacuum to give the crude product, which was purified by preparative high-performance liquid chromatography over Column: Boston prime C18 150*30 mm*5 um to give the title compound N-(2-(4,4-difluoropiperidin-1-yl)ethyl)-4-methyl-3-((1-methyl-6-((1-methyl-1H-pyrazol-4-yl)amino)-1H-pyrazolo[3,4-d]pyrimidin-3-yl)amino)benzamide (55.5 mg, 39%) as a yellow solid. LCMS (ESI): mass calcd. for C₂₅H₃₀F₂N₁₀O, 524.26; m/z found, 525.4 [M+H]⁺. ¹H NMR (400 MHz, METHANOL-d₄) δ 8.67 (s, 1H), 8.13 (d, J=1.3 Hz, 1H), 8.09 (s, 1H), 7.64 (s, 1H), 7.44-7.39 (m, 1H), 7.31 (d, J=7.9 Hz, 1H), 3.91 (s, 3H), 3.81 (s, 3H), 3.51 (t, J=6.7 Hz, 2H), 2.68-2.62 (m, 6H), 2.40 (s, 3H), 2.03-1.93 (m, 4H).

### Example 119. (S)-4-fluoro-3-((1-methyl-6-((1-methyl-1H-pyrazol-4-yl)amino)-1H-pyrazolo[3,4-d]pyrimidin-3-yl)amino)-N-(2-(2-methylpyrrolidin-1-yl)ethyl)benzamide

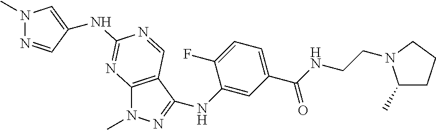 [SMILES: C[C@H]1CCCN1CCNC(=O)c1ccc(F)c(Nc2nn(C)c3nc(Nc4cnn(C)c4)ncc23)c1]

To a solution of 4-fluoro-3-((1-methyl-6-((1-methyl-1H-pyrazol-4-yl)amino)-1H-pyrazolo[3,4-d]pyrimidin-3-yl)amino)benzoic acid (80 mg, 0.144 mmol), HATU (110 mg, 0.29 mmol) and N,N-diisopropylethylamine (74 mg, 0.58 mmol) in N,N-dimethylformamide (3 mL) was added (S)-2-(2-methylpyrrolidin-1-yl)ethanamine (18.4 mg, 0.14 mmol). The mixture stirred at 25° C. for 2 h and then concentrated under vacuum to give the crude product, which was purified by preparative high-performance liquid chromatography over Column: Boston prime C18 150*30 mm*5 um to give the title compound (S)-4-fluoro-3-((1-methyl-6-((1-methyl-1H-pyrazol-4-yl)amino)-1H-pyrazolo[3,4-d]pyrimidin-3-yl)amino)-N-(2-(2-methylpyrrolidin-1-yl)ethyl)benzamide (25 mg, 34%) as a white solid. LCMS (ESI): mass calcd. for C₂₄H₂₉FN₁₀O, 492.25; m/z found, 493.3 [M+H]⁺. ¹H NMR (400 MHz, METHANOL-d₄) δ 8.92 (s, 1H), 8.75 (dd, J=8.16, 2.13 Hz, 1H), 8.11 (s, 1H), 7.66 (s, 1H), 7.37-7.42 (m, 1H), 7.23 (dd, J=11.04, 8.53 Hz, 1H), 3.93 (s, 3H), 3.89 (s, 3H), 3.61-3.69 (m, 1H), 3.46-3.55 (m, 1H), 3.09-3.16 (m, 1H), 2.43-2.54 (m, 1H), 2.29-2.43 (m, 3H), 1.97-2.08 (m, 1H), 1.81 (br d, J=8.28 Hz, 2H), 1.40-1.52 (m, 1H), 1.17 (d, J=6.02 Hz, 3H).

### Example 120. N-(2-(3,3-dimethylazetidin-1-yl)ethyl)-4-fluoro-3-((1-methyl-6-((1-methyl-1H-pyrazol-4-yl)amino)-1H-pyrazolo[3,4-d]pyrimidin-3-yl)amino)benzamide

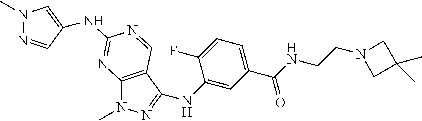

### Step a: N-(2-(3,3-dimethylazetidin-1-yl)ethyl)-4-fluoro-3-((1-methyl-6-((1-methyl-1H-pyrazol-4-yl)amino)-1H-pyrazolo[3,4-d]pyrimidin-3-yl)amino)benzamide

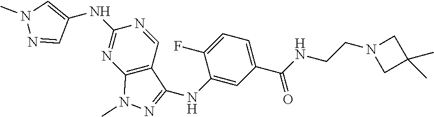

To a solution of 4-fluoro-3-((1-methyl-6-((1-methyl-1H-pyrazol-4-yl)amino)-1H-pyrazolo[3,4-d]pyrimidin-3-yl)amino)benzoic acid (80 mg, 0.144 mmol), HATU (109.4 mg, 0.288 mmol) and N,N-diisopropylethylamine (74.4 mg, 0.58 mmol) in N,N-dimethylformamide (2 mL) was added 2-(3,3-dimethylazetidin-1-yl)ethanamine (18.4 mg, 0.14 mmol). The mixture stirred at 25° C. for 2 h and then concentrated under vacuum to give the crude product, which was purified by preparative high-performance liquid chromatography over Column: Boston prime C18 150*30 mm*5 um to give the title compound N-(2-(3,3-dimethylazetidin-1-yl)ethyl)-4-fluoro-3-((1-methyl-6-((1-methyl-1H-pyrazol-4-yl)amino)-1H-pyrazolo[3,4-d]pyrimidin-3-yl)amino)benzamide (32.2 mg, 43%) as a white solid. LCMS (ESI): mass calcd. for C₂₄H₂₉FN₁₀O, 492.25; m/z found, 493.3 [M+H]⁺. ¹H NMR (400 MHz, METHANOL-d₄) δ 8.94 (s, 1H), 8.78 (dd, J=8.23, 2.15 Hz, 1H), 8.10 (s, 1H), 7.68 (s, 1H), 7.43 (ddd, J=8.37, 4.44, 2.21 Hz, 1H), 7.25 (dd, J=11.09, 8.46 Hz, 1H), 3.93 (s, 3H), 3.90 (s, 3H), 3.74 (s, 4H), 3.57 (t, J=5.96 Hz, 2H), 3.23 (br t, J=5.78 Hz, 2H), 1.35 (s, 6H).

### Example 121. N-(2-(2,2-dimethylpyrrolidin-1-yl)ethyl)-4-fluoro-3-((1-methyl-6-((1-methyl-1H-pyrazol-4-yl)amino)-1H-pyrazolo[3,4-d]pyrimidin-3-yl)amino)benzamide

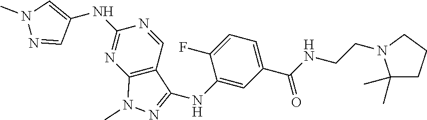 [SMILES: Cn1cc(Nc2ncc3c(Nc4cc(C(=O)NCCN5CCCC5(C)C)ccc4F)nn(C)c3n2)cn1]

### Step a: N-(2-(2,2-dimethylpyrrolidin-1-yl)ethyl)-4-fluoro-3-((1-methyl-6-((1-methyl-1H-pyrazol-4-yl)amino)-1H-pyrazolo[3,4-d]pyrimidin-3-yl)amino)benzamide

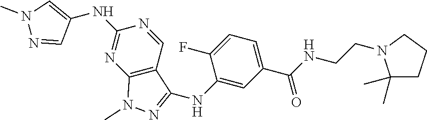 [SMILES: Cn1cc(Nc2ncc3c(Nc4cc(C(=O)NCCN5CCCC5(C)C)ccc4F)nn(C)c3n2)cn1]

To a solution of 4-fluoro-3-((1-methyl-6-((1-methyl-1H-pyrazol-4-yl)amino)-1H-pyrazolo[3,4-d]pyrimidin-3-yl)amino)benzoic acid (80 mg, 0.144 mmol), HATU (109.4 mg, 0.29 mmol) and N,N-diisopropylethylamine (74 mg, 0.58 mmol) in N,N-dimethylformamide (2 mL) was added 2-(2,2-dimethylpyrrolidin-1-yl)ethanamine (18 mg, 0.15 mmol). The mixture stirred at 25° C. for 2 h and then concentrated under vacuum to give the crude product, which was purified by preparative high-performance liquid chromatography over Column: Boston prime C18 150*30 mm*5 um to give the title compound N-(2-(3,3-dimethylazetidin-1-yl)ethyl)-4-fluoro-3-((1-methyl-6-((1-methyl-1H-pyrazol-4-yl)amino)-1H-pyrazolo[3,4-d]pyrimidin-3-yl)amino)benzamide (25 mg, 33%) as a white solid. LCMS (ESI): mass calcd. for C₂₅H₃₁FN₁₀O, 506.27; m/z found, 507.2 [M+H]⁺. ¹H NMR (400 MHz, METHANOL-d₄) δ 9.13 (s, 1H), 8.95 (dd, J=8.23, 2.03 Hz, 1H), 8.31 (s, 1H), 7.87 (s, 1H), 7.55-7.65 (m, 1H), 7.43 (dd, J=11.09, 8.46 Hz, 1H), 4.13 (s, 3H), 4.10 (s, 3H), 3.73 (br t, J=6.97 Hz, 2H), 3.13 (br t, J=7.33 Hz, 2H), 2.89 (br t, J=6.91 Hz, 2H), 2.00-2.12 (m, 2H), 1.89-1.94 (m, 2H), 1.27 (s, 6H).

### Example 122. N-(2-(5-azaspiro[2.4]heptan-5-yl)ethyl)-4-fluoro-3-((1-methyl-6-((1-methyl-1H-pyrazol-4-yl)amino)-1H-pyrazolo[3,4-d]pyrimidin-3-yl)amino)benzamide

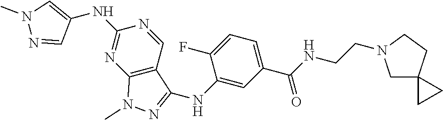 [SMILES: Cn1cc(Nc2ncc3c(Nc4cc(C(=O)NCCN5CCC6(CC6)C5)ccc4F)nn(C)c3n2)cn1]

To a solution of 4-fluoro-3-((1-methyl-6-((1-methyl-1H-pyrazol-4-yl)amino)-1H-pyrazolo[3,4-d]pyrimidin-3-yl)amino)benzoic acid (80 mg, 0.14 mmol), HATU (109 mg, 0.29 mmol) and N,N-diisopropylethylamine (74 mg, 0.575 mmol) in N,N-dimethylformamide (2 mL) was added 2-(5-azaspiro[2.4]heptan-5-yl)ethanamine (18 mg, 0.144 mmol). The mixture stirred at 25° C. for 2 h and then concentrated under vacuum to give the crude product, which was purified by preparative high-performance liquid chromatography over Column: Boston prime C18 150*30 mm*5 um to give the title compound N-(2-(5-azaspiro[2.4]heptan-5-yl)ethyl)-4-fluoro-3-((1-methyl-6-((1-methyl-1H-pyrazol-4-yl)amino)-1H-pyrazolo[3,4-d]pyrimidin-3-yl)amino)benzamide (25 mg, 33%) as a white solid. LCMS (ESI): mass calcd. for C₂₅H₂₉FN₁₀O, 504.25; m/z found, 505.2[M+H]⁺. ¹H NMR (400 MHz, METHANOL-d₄) δ 8.93 (br s, 1H), 8.76 (br d, J=8.46 Hz, 1H), 8.11 (br s, 1H), 7.67 (br s, 1H), 7.40 (br s, 1H), 7.23 (br t, J=9.54 Hz, 1H), 3.91 (br d, J=13.23 Hz, 6H), 3.57 (br s, 2H), 2.87 (br d, J=6.56 Hz, 2H), 2.78 (br s, 2H), 2.65 (br s, 2H), 1.88 (br s, 2H), 0.61 (br d, J=14.31 Hz, 4H).

### Example 123. N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(1-azaspiro[3.3]heptan-1-yl)acetamide

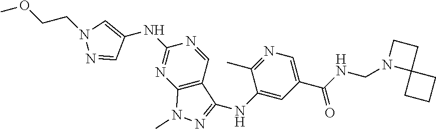 [SMILES: COCCn1cc(Nc2ncc3c(Nc4cc(C(=O)NCN5CCC56CCC6)cnc4C)nn(C)c3n2)cn1]

To a solution of 2-chloro-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide (130 mg, 0.28 mmol) and 1-azaspiro[3.3]heptane hemioxalate (117 mg, 0.41 mmol) in DMF (6 mL) was added K₂CO₃ (117 mg, 0.85 mmol) and sodium iodide (46 mg, 0.31 mmol). The reaction mixture was stirred at 50° C. for 2 hrs. The reaction mixture was purified by preparative high-performance liquid chromatography over column: Agela DuraShell C18 250*80 mm*10 um to give the title compound N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(1-azaspiro[3.3]heptan-1-yl)acetamide (21 mg, 14%) as a white solid. LCMS (ESI): mass calcd. for C₂₆H₃₃N₁₁O₂, 531.3; m/z found, 532.3 [M+H]⁺. ¹H NMR (400 MHz, METHANOL-d₄) δ 8.85 (s, 1H), 8.64 (s, 1H), 8.36 (s, 1H), 8.18 (br s, 1H), 7.72 (s, 1H), 4.32 (br t, J=5.0 Hz, 2H), 3.86 (s, 3H), 3.79 (br t, J=5.0 Hz, 2H), 3.54 (s, 2H), 3.49 (br t, J=7.0 Hz, 2H), 3.37 (s, 3H), 2.57 (s, 3H), 2.46-2.34 (m, 4H), 2.08 (br s, 2H), 1.79-1.67 (m, 2H)

### Example 124. N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(5-azaspiro[2.4]heptan-5-yl)acetamide

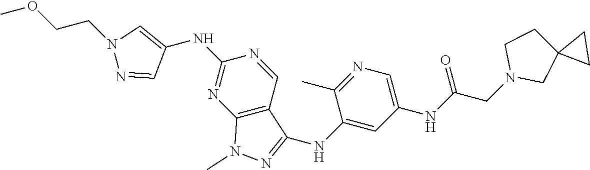 [SMILES: COCCn1cc(Nc2ncc3c(Nc4cc(NC(=O)CN5CCC6(CC6)C5)cnc4C)nn(C)c3n2)cn1]

To a solution of 2-chloro-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide (130 mg, 0.18 mmol) in DMF (4 mL) was added 5-azaspiro[2.4]heptane hydrochloride (78 mg, 0.58 mmol), K₂CO₃ (117 mg, 0.85 mmol) and sodium iodide (46 mg, 0.31 mmol). The mixture was stirred at 50° C. overnight. The reaction mixture was purified by column chromatography over silica gel (eluent: dichloromethane/methanol=100:0 to 30:70). The desired fractions were collected, and the solvent was concentrated under vacuum to give orange solid. The orange solid was purified by preparative high-performance liquid chromatography over column: Phenomenex C18 80*40 mm*3 um to give the title compound N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(5-azaspiro[2.4]heptan-5-yl)acetamide (16 mg, 17%) as an off-white solid. LCMS (ESI): mass calcd. for C₂₆H₃₃N₁₁O₂, 531.3; m/z found, 532.3 [M+H]⁺. ¹H NMR (400 MHz, METHANOL-d₄) δ 8.82-8.90 (m, 1H), 8.62-8.69 (m, 1H), 8.37-8.46 (m, 1H), 8.15-8.25 (m, 1H), 7.67-7.75 (m, 1H), 4.63 (s, 6H), 4.33 (t, J=5.13 Hz, 2H), 3.79 (t, J=5.19 Hz, 2H), 3.59 (s, 2H), 3.07-3.13 (m, 2H), 2.84-2.89 (m, 2H), 2.53-2.61 (m, 3H), 1.95 (t, J=7.03 Hz, 2H), 0.67 (d, J=2.26 Hz, 2H), 0.63-0.66 (m, 2H).

### Example 125. 2-(2,2-dimethylpyrrolidin-1-yl)-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide

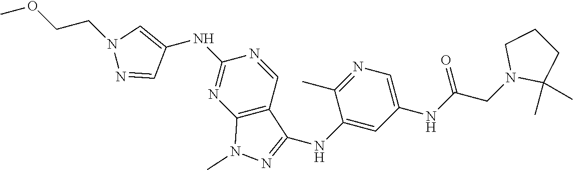

To the mixture of 2-chloro-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide (50 mg, 0.08 mmol) in DMF (3 mL) was added 2,2-dimethylpyrrolidine (18 mg, 0.18 mmol), K₂CO₃ (48 mg, 0.35 mmol) and sodium iodide (11 mg, 0.07 mmol) at room temperature. The resulting mixture was heated at 50° C. for 2 hours before cooling to room-temperature. The reaction mixture was filtered and the filtrate was concentrated under vacuum to give crude product, which was purified by preparative high-performance liquid chromatography over column: Phenomenex C18 80*40 mm*3 um to give the title compound 2-(2,2-dimethylpyrrolidin-1-yl)-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide (24 mg, 53%) as a white solid. LCMS (ESI): mass calcd. for C₂₆H₃₅N₁₁O₂, 533.6; m/z found, 534.3 [M+H]⁺. ¹H NMR (400 MHz, METHANOL-d₄) δ ppm 8.85 (s, 1H), 8.68 (d, J=2.26 Hz, 1H), 8.34 (d, J=2.26 Hz, 1H), 8.19 (s, 1H), 7.71 (s, 1H), 4.32 (t, J=5.14 Hz, 2H), 3.86 (s, 3H), 3.78 (t, J=5.14 Hz, 2H), 3.37 (s, 3H), 2.97 (br s, 2H), 2.57 (s, 3H), 1.85-1.96 (m, 2H), 1.76-1.84 (m, 2H), 1.23-1.44 (m, 2H), 1.14 (s, 6H)

### Example 126. 2-(3,3-dimethylazetidin-1-yl)-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide

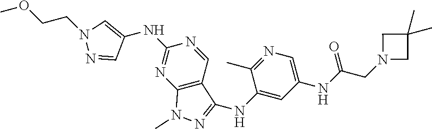

To a solution of 2-chloro-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide (80 mg, 0.16 mmol), 3,3-dimethylazetidine hydrochloride (24 mg, 0.20 mmol), K₂CO₃ (56 mg, 0.41 mmol) and sodium iodide (12 mg, 0.08 mmol) in DMF (4 mL) at room temperature. The resulting mixture was heated at 50° C. for 2 hours before cooling to room-temperature. The reaction mixture was filtered and the filtrate was concentrated under vacuum to give crude product, which was purified by preparative high-performance liquid chromatography over column: Phenomenex C18 80*40 mm*3 um to give the title compound 2-(3,3-dimethylazetidin-1-yl)-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide (32 mg, 39%) as a yellow solid. LCMS (ESI): mass calcd. for C₂₅H₃₃N₁₁O₂, 519.6; m/z found, 520.3 [M+H]⁺. ¹H NMR (400 MHz, METHANOL-d₄) δ ppm 8.85 (s, 1H), 8.63 (d, J=2.15 Hz, 1H), 8.36 (d, J=2.27 Hz, 1H), 8.19 (s, 1H), 7.71 (s, 1H), 4.32 (t, J=5.19 Hz, 2H), 3.86 (s, 3H), 3.78 (t, J=5.19 Hz, 2H), 3.37 (s, 3H), 3.36 (s, 2H), 3.21 (s, 4H), 2.56 (s, 3H), 1.28 (s, 6H)

### Example 127. 2-(2-azabicyclo[2.2.2]octan-2-yl)-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide

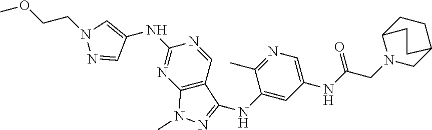 [SMILES: COCCn1cc(Nc2ncc3c(Nc4cc(NC(=O)CN5CC6CCC5CC6)cnc4C)nn(C)c3n2)cn1]

To the mixture of 2-chloro-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide (130 mg, 0.28 mmol) in DMF (4 mL) was added 2-azabicyclo[2.2.2]octane hydrochloride (134 mg, 0.91 mmol), K₂CO₃ (243 mg, 1.7 mmol) and sodium iodide (62 mg, 0.41 mmol). The mixture was stirred at 50° C. overnight and then concentrated under vacuum to give the crude product, which was purified by preparative high-performance liquid chromatography over column: Welch Xtimate C18 150*25 mm*5 um to give the title compound 2-(2-azabicyclo[2.2.2]octan-2-yl)-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide (15.5 mg, 10%) as a white solid. LCMS (ESI): mass calcd. for C₂₇H₃₅N₁₁O₂, 545.6; m/z found, 546.3 [M+H]⁺. ¹H NMR (400 MHz, METHANOL-d₄) δ 8.85 (s, 1H), 8.68 (d, J=2.1 Hz, 1H), 8.35 (d, J=2.3 Hz, 1H), 8.18 (s, 1H), 7.71 (s, 1H), 4.32 (t, J=5.2 Hz, 2H), 3.86 (s, 3H), 3.78 (t, J=5.1 Hz, 2H), 3.37 (s, 5H), 2.91 (s, 2H), 2.62 (br s, 1H), 2.57 (s, 3H), 2.06 (br d, J=11.8 Hz, 2H), 1.77 (br s, 1H), 1.74-1.68 (m, 2H), 1.66-1.57 (m, 4H)

### Example 128. (S)—N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(2-methylpyrrolidin-1-yl)acetamide

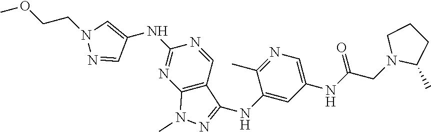

To a solution of 2-chloro-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide (100 mg, 0.18 mmol) and (S)-1,2-dimethylpyrrolidine (20 mg, 0.24 mmol) in DMF (5 mL) was added K₂CO₃ (70 mg, 0.51 mmol) and sodium iodide (15 mg, 0.1 mmol) at room temperature. The reaction mixture was stirred at 50° C. for 2 hours. The reaction mixture was filtered. The filtrate was concentrated under vacuum to give the crude product, which was purified by preparative high-performance liquid chromatography over column: Welch Xtimate C18 150*30 mm*5 um to give the title compound (S)—N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(2-methylpyrrolidin-1-yl)acetamide (25.4 mg, 27%) as a yellow solid. LCMS (ESI): mass calcd. for C₂₅H₃₃N₁₁O₂, 519.602; m/z found, 520.3 [M+H]⁺. ¹H NMR (400 MHz, CHLOROFORM-d) δ 8.85 (s, 1H), 8.66 (d, J=2.15 Hz, 1H), 8.36 (d, J=2.15 Hz, 1H), 8.19 (s, 1H), 7.71 (s, 1H), 4.33 (t, J=5.19 Hz, 2H), 3.86 (s, 3H), 3.79 (t, J=5.19 Hz, 2H), 3.57 (d, J=15.85 Hz, 1H), 3.37 (s, 3H), 3.24-3.29 (m, 1H), 3.12 (d, J=15.97 Hz, 1H), 2.59-2.66 (m, 1H), 2.57 (s, 3H), 2.42 (q, J=8.82 Hz, 1H), 1.97-2.10 (m, 1H), 1.73-1.94 (m, 2H), 1.46-1.59 (m, 1H), 1.18 (d, J=6.08 Hz, 3H)

### Example 129. 2-(3,3-dimethylpyrrolidin-1-yl)-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide

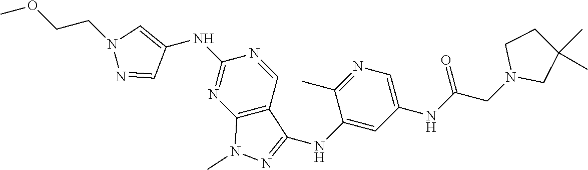

To a solution of 2-chloro-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide (120 mg, 0.26 mmol) in DMF (3 mL) was added 3,3-dimethylpyrrolidine hydrochloride (44 mg, 0.32 mmol), K₂CO₃ (83 mg, 0.6 mmol) and sodium iodide (18 mg, 0.12 mmol). The resulting mixture was stirred at 50° C. for 12 h. The resulting mixture was purified by column chromatography over silica gel (eluent: DCM/MeOH from 100:0 to 90:10) to give the crude product. Which was purified by preparative high-performance liquid chromatography over column: Phenomenex Gemini-NX 75*30 mm*3 um to give the crude product and the crude product was purified by Supercritical Fluid Chromatography over column: Welch Xtimate C18 150*30 mm*5 um to give title compound 2-(3,3-dimethylpyrrolidin-1-yl)-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide (18.4 mg, 13%) as a white solid. LCMS (ESI): mass calcd. for C₂₆H₃₅N₁₁O₂, 533.6; m/z found, 534.3 [M+H]⁺. ¹H NMR (400 MHz, CHLOROFORM-d) δ ppm 9.25 (br s, 1H), 8.64 (br d, J=8.46 Hz, 2H), 8.14-8.22 (m, 1H), 8.04 (s, 1H), 7.68 (s, 1H), 7.07-7.17 (m, 1H), 6.18 (s, 1H), 4.33 (br t, J=5.01 Hz, 2H), 3.90 (s, 3H), 3.81 (br t, J=5.13 Hz, 2H), 3.40 (s, 3H), 3.27 (s, 2H), 2.84 (br t, J=7.15 Hz, 2H), 2.58 (s, 3H), 2.50 (s, 2H), 1.69-1.72 (m, 2H), 1.17 (s, 6H).

### Example 130. N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(1-azaspiro[3.3]heptan-1-yl)acetamide

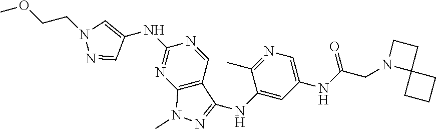 [SMILES: COCCn1cc(Nc2ncc3c(Nc4cc(NC(=O)CN5CCC56CCC6)cnc4C)nn(C)c3n2)cn1]

To a solution of 2-chloro-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide (130 mg, 0.28 mmol) and 1-azaspiro[3.3]heptane hemioxalate (117 mg, 0.41 mmol) in DMF (6 mL) was added K₂CO₃ (117 mg, 0.85 mmol) and sodium iodide (46 mg, 0.31 mmol). The reaction mixture was stirred at 50° C. for 2 hrs. The reaction mixture was purified by preparative high-performance liquid chromatography over column: Agela DuraShell C18 250*80 mm*10 um to give the title compound N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(1-azaspiro[3.3]heptan-1-yl)acetamide (21 mg, 14%) as a white solid. LCMS (ESI): mass calcd. for C₂₆H₃₃N₁₁O₂, 531.3; m/z found, 532.3 [M+H]⁺. ¹H NMR (400 MHz, METHANOL-d₄) δ 8.85 (s, 1H), 8.64 (s, 1H), 8.36 (s, 1H), 8.18 (br s, 1H), 7.72 (s, 1H), 4.32 (br t, J=5.0 Hz, 2H), 3.86 (s, 3H), 3.79 (br t, J=5.0 Hz, 2H), 3.54 (s, 2H), 3.49 (br t, J=7.0 Hz, 2H), 3.37 (s, 3H), 2.57 (s, 3H), 2.46-2.34 (m, 4H), 2.08 (br s, 2H), 1.79-1.67 (m, 2H)

### Example 131. N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(5-azaspiro[2.4]heptan-5-yl)acetamide

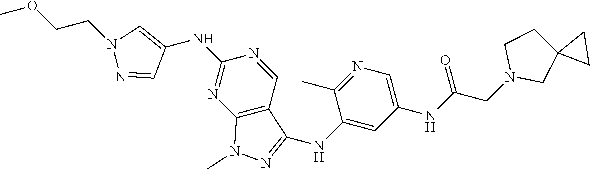 [SMILES: COCCn1cc(Nc2ncc3c(Nc4cc(NC(=O)CN5CCC6(CC6)C5)cnc4C)nn(C)c3n2)cn1]

To a solution of 2-chloro-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide (130 mg, 0.18 mmol) in DMF (4 mL) was added 5-azaspiro[2.4]heptane hydrochloride (78 mg, 0.58 mmol), K₂CO₃ (117 mg, 0.85 mmol) and sodium iodide (46 mg, 0.31 mmol). The mixture was stirred at 50° C. overnight. The reaction mixture was purified by column chromatography over silica gel (eluent: dichloromethane/methanol=100:0 to 30:70). The desired fractions were collected, and the solvent was concentrated under vacuum to give orange solid. The orange solid was purified by preparative high-performance liquid chromatography over column: Phenomenex C18 80*40 mm*3 um to give the title compound N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(5-azaspiro[2.4]heptan-5-yl)acetamide (16 mg, 17%) as an off-white solid. LCMS (ESI): mass calcd. for C₂₆H₃₃N₁₁O₂, 531.3; m/z found, 532.3 [M+H]⁺. ¹H NMR (400 MHz, METHANOL-d₄) δ 8.82-8.90 (m, 1H), 8.62-8.69 (m, 1H), 8.37-8.46 (m, 1H), 8.15-8.25 (m, 1H), 7.67-7.75 (m, 1H), 4.63 (s, 6H), 4.33 (t, J=5.13 Hz, 2H), 3.79 (t, J=5.19 Hz, 2H), 3.59 (s, 2H), 3.07-3.13 (m, 2H), 2.84-2.89 (m, 2H), 2.53-2.61 (m, 3H), 1.95 (t, J=7.03 Hz, 2H), 0.67 (d, J=2.26 Hz, 2H), 0.63-0.66 (m, 2H).

### Example 132. N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(5-azaspiro[3.4]octan-5-yl)acetamide

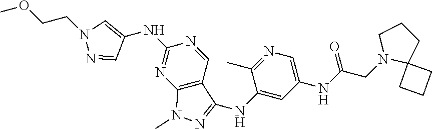 [SMILES: COCCn1cc(Nc2ncc3c(Nc4cc(NC(=O)CN5CCCC56CCC6)cnc4C)nn(C)c3n2)cn1]

To a solution of 2-chloro-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl) acetamide (130 mg, 0.19 mmol) in DMF (8 mL) was added 5-azaspiro [3.4]octane (60 mg, 0.43 mmol), K₂CO₃ (110 mg, 0.80 mmol) and sodium iodide (30 mg, 0.17 mmol) at room-temperature. The resulting mixture was stirred at 50° C. for 2 h before cooling to room-temperature. The reaction mixture was filtered and the filtrate was concentrated under vacuum to give crude product, which was purified by preparative high-performance liquid chromatography over column: Phenomenex C18 80*40 mm*3 um to give the title compound N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(5-azaspiro[3.4]octan-5-yl)acetamide (15.1 mg, 15%) as a white solid. LCMS (ESI): mass calcd. for C₂₇H₃₅N₁₁O₂ 545.6; m/z found, 546.5 [M+H]⁺. ¹H NMR (400 MHz, CD₃OD) δ 8.91-8.79 (m, 1H), 8.73-8.55 (m, 1H), 8.42-8.26 (m, 1H), 8.25-8.08 (m, 1H), 7.79-7.62 (m, 1H), 4.36-4.29 (m, 2H), 3.92-3.82 (m, 3H), 3.82-3.74 (m, 2H), 3.44 (br s, 2H), 3.37 (br s, 3H), 2.90-2.77 (m, 2H), 2.63-2.49 (m, 3H), 2.29-2.18 (m, 2H), 2.09-2.02 (m, 2H), 1.91-1.76 (m, 4H), 1.76-1.63 (m, 2H).

### Example 133. 2-(cyclopentyl(2-fluoroethyl)amino)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

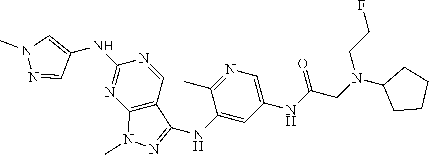 [SMILES: Cc1ncc(NC(=O)CN(CCF)C2CCCC2)cc1Nc1nn(C)c2nc(Nc3cnn(C)c3)ncc12]

### Step a: N-(2-fluoroethyl)cyclopentanamine

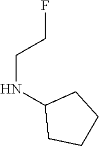

A mixture of cyclopentanone (2 g, 23.7 mmol), 2-fluoroethanamine (2.6 g, 26.2 mmol) and AcOH (2.8 g, 47.5 mmol) in MeOH (30 mL), then NaBH₃CN (2.9 g, 47.5 mmol) was added at 0° C. The reaction mixture was stirred at 25° C. for 16 hours. The mixture was concentrated in vacuo to give the crude product. The product was purified by silica gel chromatography (petroleum ether/ethyl acetate from 100/0 to 0/100) to give the title compound N-(2-fluoroethyl)cyclopentanamine (200 mg, 6.4%) as a yellow oil.

### Step b: 2-(cyclopentyl(2-fluoroethyl)amino)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

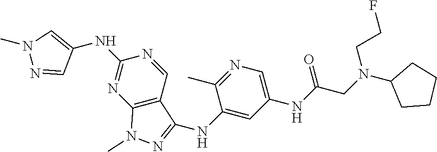 [SMILES: Cc1ncc(NC(=O)CN(CCF)C2CCCC2)cc1Nc1nn(C)c2nc(Nc3cnn(C)c3)ncc12]

To a mixture of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (80 mg, 0.18 mmol), K₂CO₃ (155 mg, 1.1 mmoL) and sodium iodide (93 mg, 0.62 mmol) in DMF (5 mL) was added N-(2-fluoroethyl)cyclopentanamine (74 mg, 0.56 mmol). The reaction mixture was stirred at 50° C. for 2 h before cooling to room-temperature. The reaction mixture was filtered and the filtrate was concentrated under vacuum to give crude product, which was purified by preparative high-performance liquid chromatography over column: Welch Xtimate C18 150*30 mm*5 um to give the title compound 2-(cyclopentyl(2-fluoroethyl)amino)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (26 mg, 26%) as a white solid. LCMS (ESI): mass calcd. for C₂₅H₃₂FN₁₁O, 521.6; m/z found, 522.4 [M+H]⁺. ¹H NMR (400 MHz, CHLOROFORM-d) δ 8.83 (s, 111), 8.67 (d, J=2.0 Hz, 1H), 8.28 (d, J=2.3 Hz, 1H), 8.07 (s, 1H), 7.65 (s, 1H), 4.64 (t, J=4.8 Hz, 2H), 4.52 (t, J=4.9 Hz, 1H), 3.90 (s, 3H), 3.83 (s, 3H), 3.36 (s, 2H), 3.04 (t, J=4.8 Hz, 1H), 2.97 (t, J=4.8 Hz, 1H), 2.54 (s, 3H), 1.89 (br d, J=6.8 Hz, 2H), 1.75-1.66 (m, 2H), 1.59 (br dd, J=4.6, 7.4 Hz, 2H), 1.51-1.41 (m, 2H).

### Example 134. 2-(4-methoxypiperidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

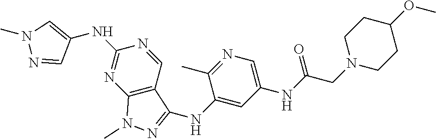

To a mixture of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (90 mg, 0.17 mmol), K₂CO₃ (72 mg, 0.52 mmoL) and sodium iodide (36 mg, 0.24 mmol) in DMF (2 mL) was added 4-methoxypiperidine (63 mg, 0.55 mmol). The reaction mixture was stirred at 50° C. for 2 h before cooling to room-temperature. The reaction mixture was filtered and the filtrate was concentrated under vacuum to give crude product, which was purified by preparative by preparative high-performance liquid chromatography over column: Boston Prime C18 150*30 mm*5 um to give the title compound 2-(4-methoxypiperidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (57 mg, 62%) as a white solid. LCMS (ESI): mass calcd. for C₂₄H₃₁N₁₁O₂, 505.6; m/z found, 506.3 [M+H]⁺. ¹H NMR (400 MHz, METHANOL-d₄) δ 8.82 (s, 1H), 8.67 (d, J=1.91 Hz, 1H), 8.32 (d, J=2.03 Hz, 1H), 8.06 (s, 1H), 7.65 (s, 1H), 3.90 (s, 3H), 3.83 (s, 3H), 3.35-3.37 (m, 3H), 3.28-3.32 (m, 1H), 3.19 (s, 2H), 2.80-2.88 (m, 2H), 2.53 (s, 3H), 2.37-2.44 (m, 2H), 1.94-2.01 (m, 2H), 1.64-1.73 (m, 2H).

### Example 135. (R)-2-(3-methoxypyrrolidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

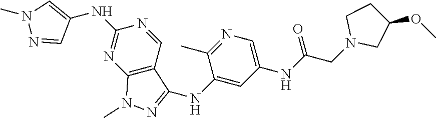

To a solution of 2-chloro-N-(6-methyl-5-((1-methyl-6-((1-methyl-1H-pyrazol-4-yl)amino)-1H-pyrazolo[3,4-d]pyrimidin-3-yl)amino)-276-pyridine-3-yl)acetamide (100 mg, 0.20 mmol) and ®-3-methoxypyrrolidine hydrochloride (82 mg, 0.60 mmol) in DMF (6 mL) was added K₂CO₃ (110 mg, 0.80 mmol) and sodium iodide (35 mg, 0.23 mmol). The reaction mixture was stirred at 50° C. for 1.5 hrs. The reaction mixture was purified by preparative high-performance liquid chromatography over column: Phenomenex C18 80*40 mm*3 um to give the title compound (R)-2-(3-methoxypyrrolidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (31 mg, 31%) as an off-white solid. LCMS (ESI): mass calcd. for C₂₃H₂₉N₁₁O₂, 491.3; m/z found, 492.3 [M+H]⁺. ¹H NMR (400 MHz, METHANOL-d₄) δ 8.83 (s, 1H), 8.66 (d, J=2.0 Hz, 1H), 8.37 (d, J=2.1 Hz, 1H), 8.09 (s, 1H), 7.67 (s, 1H), 4.02 (dt, J=2.7, 4.9 Hz, 1H), 3.92 (s, 3H), 3.85 (s, 3H), 3.41-3.36 (m, 1H), 3.34 (s, 3H), 3.32-3.30 (m, 1H), 3.01-2.86 (m, 2H), 2.76 (dd, J=5.4, 10.4 Hz, 1H), 2.61-2.51 (m, 4H), 2.19 (dt, J=7.8, 13.5 Hz, 1H), 1.95-1.84 (m, 1H).

### Example 136. (S)-2-(3-methoxypyrrolidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

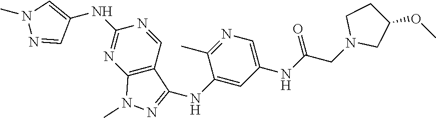

To a solution of 2-chloro-N-(6-methyl-5-((1-methyl-6-((1-methyl-1H-pyrazol-4-yl)amino)-1H-pyrazolo[3,4-d]pyrimidin-3-yl)amino)-276-yridine-3-yl)acetamide (150 mg, 0.30 mmol), potassium carbonate (122 mg, 0.89 mmol), sodium iodide (48.7 mg, 0.33 mmol) in DMF (6 mL) were added (S)-3-methoxypyrrolidine (89.5 mg, 0.89 mmol) at room temperature. The reaction was stirred at 50° C. for 2 h. The reaction mixture was concentrated in vacuo to give the crude product, which was purified by preparative high-performance liquid chromatography over column: Boston Green ODS 150*30 mm*5 um to give the title compound (S)-2-(3-methoxypyrrolidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (57.3 mg, 37%) as a white solid. LCMS (ESI): mass calcd. for C₂₃H₂₉N₁₁O, 491.5; m/z found, 492.4 [M+H]⁺. ¹H NMR (400 MHz, METHANOL-d₄) δ 8.82 (s, 1H), 8.66 (s, 1H), 8.35 (d, J=1.8 Hz, 1H), 8.04 (s, 1H), 7.65 (s, 1H), 4.10 (br s, 1H), 3.90 (s, 3H), 3.82 (s, 3H), 3.76 (br d, J=1.7 Hz, 1H), 3.39-3.34 (m, 3H), 3.33-3.30 (m, 1H), 3.28-3.19 (m, 2H), 3.10 (br dd, J=4.8, 11.4 Hz, 1H), 3.04-2.95 (m, 1H), 2.53 (s, 3H), 2.22 (qd, J=7.2, 14.0 Hz, 1H), 2.06 (br dd, J=6.3, 13.4 Hz, 1H)

### Example 137. 2-(3-methoxyazetidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

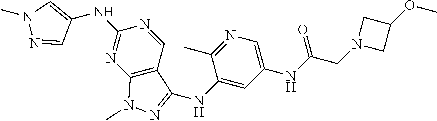

To a solution of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (90 mg, 0.18 mmol) in DMF (2 mL) was added 3-methoxyazetidine (66 mg, 0.53 mmol), K₂CO₃ (72 mg, 0.52 mmol) and sodium iodide (36 mg, 0.24 mmol). The resulting mixture was stirred at 50° C. for 1 h. The mixture was cooled to room temperature and filtered. The filtrate was concentrated under vacuum to afford a yellow solid. Which was purified by preparative high-performance liquid chromatography over column: Boston Prime C18 150*30 mm*5 um to g give the title compound 2-(3-methoxyazetidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (28.2 mg, 32%) as a white solid. LCMS (ESI): mass calcd. for C₂₂H₂₇N₁₁O₂, 477.5; m/z found, 478.3 [M+H]⁺. ¹H NMR (400 MHz, METHANOL-d₄) δ ppm 8.83 (s, 1H), 8.63 (d, J=1.91 Hz, 1H), 8.34 (d, J=2.03 Hz, 1H), 8.08 (s, 1H), 7.66 (s, 1H), 4.12 (t, J=5.60 Hz, 1H), 3.91 (s, 3H), 3.84 (s, 3H), 3.78 (dd, J=8.23, 6.56 Hz, 2H), 3.39 (s, 2H), 3.29 (s, 3H), 3.20 (dd, J=7.75, 6.20 Hz, 2H), 2.54 (s, 3H).

### Example 138. N-(6-methyl-5-((1-methyl-6-((1-methyl-1H-pyrazol-4-yl)amino)-1H-pyrazolo[3,4-d]pyrimidin-3-yl)amino)pyridin-3-yl)-2-(pyrrolidin-1-yl)acetamide

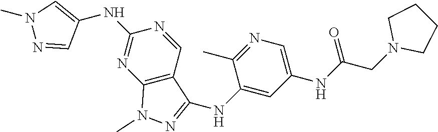 [SMILES: Cc1ncc(NC(=O)CN2CCCC2)cc1Nc1nn(C)c2nc(Nc3cnn(C)c3)ncc12]

To a mixture of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (80 mg, 0.16 mmol), K₂CO₃ (64 mg, 0.48 mmoL) and sodium iodide (12 mg, 0.08 mmol) in DMF (4 mL) was added pyrrolidine (16 mg, 0.23 mmol). The reaction mixture was stirred at 50° C. for 1.5 h before cooling to room-temperature. The reaction mixture was filtered and the filtrate was concentrated under vacuum to give crude product, which was purified by preparative high-performance liquid chromatography over column: Boston Prime C18 150*30 mm*5 um to give the title compound N-(6-methyl-5-((1-methyl-6-((1-methyl-1H-pyrazol-4-yl)amino)-1H-pyrazolo[3,4-d]pyrimidin-3-yl)amino)pyridin-3-yl)-2-(pyrrolidin-1-yl)acetamide (46.3 mg, 61%) as a yellow solid. LCMS (ESI): mass calcd. for C₂₂H₂₇N₁O, 461.5; m/z found, 462.3 [M+H]⁺. ¹H NMR (400 MHz, METHANOL-d₄) δ ppm 8.85 (s, 1H), 8.66 (d, J=2.01 Hz, 1H), 8.37 (d, J=2.26 Hz, 1H), 8.11 (s, 1H), 7.67 (s, 1H), 3.93 (s, 3H), 3.86 (s, 3H), 3.37 (s, 2H), 2.72 (br s, 4H), 2.56 (s, 3H), 1.88 (br t, J=3.39 Hz, 4H).

### Example 139. (R)-2-(3-methoxypyrrolidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide

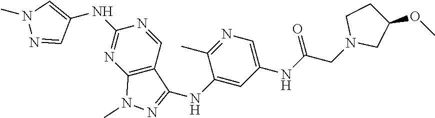

To a solution of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (100 mg, 0.20 mmol) and ®-3-methoxypyrrolidine hydrochloride (82 mg, 0.60 mmol) in DMF (6 mL) was added K₂CO₃ (110 mg, 0.80 mmol) and sodium iodide (35 mg, 0.23 mmol). The reaction mixture was stirred at 50° C. for 1.5 hrs. The reaction mixture was purified by preparative high-performance liquid chromatography over column: Phenomenex C18 80*40 mm*3 um to give the title compound (R)-2-(3-methoxypyrrolidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide (31 mg, 31%) as an off-white solid. LCMS (ESI): mass calcd. for C₂₃H₂₉N₁₁O₂, 491.3; m/z found, 492.3 [M+H]⁺. ¹H NMR (400 MHz, METHANOL-d₄) δ 8.83 (s, 1H), 8.66 (d, J=2.0 Hz, 1H), 8.37 (d, J=2.1 Hz, 1H), 8.09 (s, 1H), 7.67 (s, 1H), 4.02 (dt, J=2.7, 4.9 Hz, 1H), 3.92 (s, 3H), 3.85 (s, 3H), 3.41-3.36 (m, 1H), 3.34 (s, 3H), 3.32-3.30 (m, 1H), 3.01-2.86 (m, 2H), 2.76 (dd, J=5.4, 10.4 Hz, 1H), 2.61-2.51 (m, 4H), 2.19 (dt, J=7.8, 13.5 Hz, 1H), 1.95-1.84 (m, 1H).

### Example 140. (S)—N-(6-methyl-5-((1-methyl-6-((1-methyl-1H-pyrazol-4-yl)amino)-1H-pyrazolo[3,4-d]pyrimidin-3-yl)amino)pyridin-3-yl)-2-(3-methylpyrrolidin-1-yl)acetamide

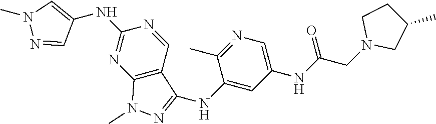

To the mixture of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl) acetamide (100 mg, 0.2 mmol) in DMF (3 mL) was added (S)-3-methylpyrrolidine hydrochloride (72 mg, 0.6 mmol), K₂CO₃ (109 mg, 0.8 mmol), sodium iodide (33 mg, 0.2 mmol). The reaction mixture was stirred at 50° C. for 1.5 hours. The reaction mixture was filtered and the filtrate was concentrated under vacuum to give crude product, which was purified by preparative high-performance liquid chromatography over column: Phenomenex C18 80*40 mm*3 um to give the title compound (S)—N-(6-methyl-5-((1-methyl-6-((1-methyl-1H-pyrazol-4-yl)amino)-1H-pyrazolo[3,4-d]pyrimidin-3-yl)amino)pyridin-3-yl)-2-(3-methylpyrrolidin-1-yl)acetamide (50.9 mg, 54%) as a white solid. LCMS (ESI): mass calcd. for C₂₃H₂₉N₁₁O 475.5; m/z found, 476.3 [M+H]⁺. ¹H NMR (400 MHz, METHANOL-d₄) δ 8.87-8.83 (m, 1H), 8.67-8.63 (m, 1H), 8.40-8.36 (m, 1H), 8.14-8.09 (m, 1H), 7.69-7.65 (m, 1H), 4.57 (s, 3H), 3.95-3.91 (m, 3H), 3.88-3.84 (m, 3H), 3.02-2.95 (m, 1H), 2.92-2.82 (m, 1H), 2.76-2.67 (m, 1H), 2.59-2.54 (m, 3H), 2.40-2.20 (m, 2H), 2.15 (s, 1H), 1.12-1.08 (m, 3H).

### Example 141. 2-(3,3-dimethylazetidin-1-yl)-N-(6-methyl-5-((1-methyl-6-((1-methyl-1H-imidazol-4-yl)amino)-1H-pyrazolo[3,4-d]pyrimidin-3-yl)amino)pyridin-3-yl)acetamide

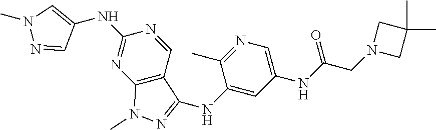

### Step a: methyl 6-methyl-5-((1-methyl-6-((1-methyl-1H-imidazol-4-yl)amino)-1H-pyrazolo[3,4-d]pyrimidin-3-yl)amino)nicotinate

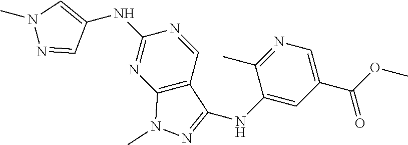

To the mixture of methyl 5-((6-chloro-1-methyl-1H-pyrazolo[3,4-d]pyrimidin-3-yl)amino)-6-methylnicotinate (1.0 g, 3.005 mmol) in DMF (15 mL) was added 1-methyl-1H-imidazol-4-amine hydrochloride (600 mg, 4.492 mmol) and Cesium Carbonate (2.940 g, 9.023 mmol), then Brettphos (810 mg, 1.509 mmol) and Brettphos-Pd-G3 (680 mg, 0.750 mmol) was added. N₂ was bubbled into the mixture for 1 min, and the resulting mixture was heated at 90° C. and stirred for 16 h. The reaction mixture was filtered through a pad of celite and the pad was washed with DCM (50 mL×3). The filtrates were concentrated to dryness in vacuo to give a black solid. The black solid was triturated with ethyl acetate (15 mL) at room temperature, then filtered. The solid was collected and dried to dryness in vacuo to give methyl 6-methyl-5-((1-methyl-6-((1-methyl-1H-imidazol-4-yl)amino)-1H-pyrazolo[3,4-d]pyrimidin-3-yl)amino)nicotinate (2.1 g, 85%) as a black solid. LCMS (ESI): mass for C₁₅H₁₉N₉O₂: 393.4; m/z found: 394.2 [M+H]⁺.

### Step b: 6-methyl-5-((1-methyl-6-((1-methyl-1H-imidazol-4-yl)amino)-1H-pyrazolo[3,4-d]pyrimidin-3-yl)amino)nicotinic acid

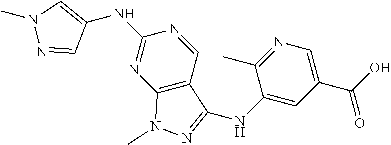 [SMILES: Cc1ncc(C(=O)O)cc1Nc1nn(C)c2nc(Nc3cnn(C)c3)ncc12]

To a solution of methyl 6-methyl-5-((1-methyl-6-((1-methyl-1H-imidazol-4-yl) amino)-1H-pyrazolo [3,4-d]pyrimidin-3-yl)amino) nicotinate (2.1 g, 2.569 mmol) in mixture of Methanol:THF:H₂O (1:1:1, 30 mL) was added LiOH (185 mg, 7.725 mmol). The mixture was stirred at room temperature for 1.5 h. The resultant was evaporated under vacuum, then adjusted to pH=3-4 with 1 mol/L HCl. The mixture was filtered under reduced pressure and the pad was washed with water (5 mL×3). The filter cake was triturated with ethyl acetate and MeOH, then filtered under reduced pressure to give 6-methyl-5-((1-methyl-6-((1-methyl-1H-imidazol-4-yl)amino)-1H-pyrazolo[3,4-d]pyrimidin-3-yl)amino)nicotinic acid (860 mg, 84%) as a brown solid. LCMS (ESI): mass for C₁₇H₁₇N₉O₂: 379.4; m/z found: 380.3 [M+H]⁺.

### Step c: tert-butyl (6-methyl-5-((1-methyl-6-((1-methyl-1H-imidazol-4-yl)amino)-1H-pyrazolo[3,4-d]pyrimidin-3-yl)amino)pyridin-3-yl)carbamate

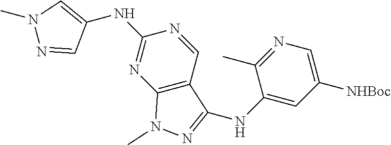 [SMILES: Cc1ncc(NC(=O)OC(C)(C)C)cc1Nc1nn(C)c2nc(Nc3cnn(C)c3)ncc12]

To the mixture of 6-methyl-5-((1-methyl-6-((1-methyl-1H-pyrazol-4-yl)amino)-1H-pyrazolo[3,4-d]pyrimidin-3-yl)amino) nicotinic acid (860 mg, 2.148 mmol) in t-BuOH (20 mL) was added triethylamine (436 mg, 4.309 mmol) and DPPA (695 μL, 3.225 mmol) at room temperature. The mixture was heated to 100° C. and stirred overnight. The reaction mixture was concentrated in vacuo to give brown oil. The brown oil was purified by flash column chromatography over a 20 g silica gel (gradient: DCM:CH₃OH from 100:0 to 90:10). The solvent was concentrated under vacuum to give tert-butyl (6-methyl-5-((1-methyl-6-((1-methyl-1H-imidazol-4-yl)amino)-1H-pyrazolo[3,4-d]pyrimidin-3-yl)amino)pyridin-3-yl)carbamate (600 mg, 42%) as a pale brown solid. LCMS (ESI): mass for C₂₁H₂₆N₁₀O₂: 450.5; m/z found: 451.3 [M+H]⁺.

### Step d: N₃-(5-amino-2-methylpyridin-3-yl)-1-methyl-N₆-(1-methyl-1H-imidazol-4-yl)-1H-pyrazolo[3,4-d]pyrimidine-3,6-diamine

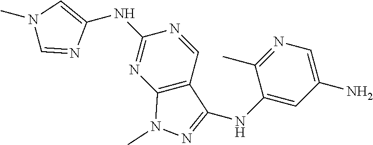 [SMILES: Cc1ncc(N)cc1Nc1nn(C)c2nc(Nc3cn(C)cn3)ncc12]

To the mixture of tert-butyl (6-methyl-5-((1-methyl-6-((1-methyl-1H-pyrazol-4-yl)amino)-1H-pyrazolo[3,4-d]pyrimidin-3-yl)amino)pyridin-3-yl) carbamate (600 mg, 0.912 mmol) in DMF (20 mL) was added HCl/dioxane (2.387 mL, 9.547 mmol, 4M) at room temperature. The reaction mixture was stirred at room temperature for 16 hours. The reaction mixture was concentrated in vacuo to give yellow gum. The yellow gum was triturated with mixture of DCM and CH₃OH (1:1, 20 mL). The mixture was filtered. The filter cake was washed with DCM. The filer cake was dried in vacuo to give (100 mg, 24%) as yellow solid. LCMS (ESI): mass for C₁₆H₁₈N₁₀: 350.4; m/z found: 351.2 [M+H]⁺.

### Step e: 2-chloro-N-(6-methyl-5-((1-methyl-6-((1-methyl-1H-imidazol-4-yl)amino)-1H-pyrazolo[3,4-d]pyrimidin-3-yl)amino)pyridin-3-yl)acetamide

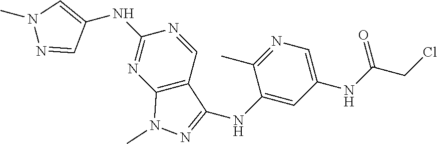 [SMILES: Cc1ncc(NC(=O)CCl)cc1Nc1nn(C)c2nc(Nc3cnn(C)c3)ncc12]

To a solution of N₃-(5-amino-2-methylpyridin-3-yl)-1-methyl-N₆-(1-methyl-1H-pyrazol-4-yl)-1H-pyrazolo[3,4-d]pyrimidine-3,6-diamine hydrochloride (100 mg, 0.208 mmol) in DMF (3 mL) was added NaHCO₃ (88 mg, 1.048 mmol). Then the mixture was cooled to 0° C. and 2-chloroacetyl chloride (27 μL, 0.318 mmoL) was added dropwise. After the reaction was warmed to room temperature, the solution was stirred for 1.5 hours. The reaction mixture was filtered. The filtrate was concentrated in vacuo to give crude. The crude was purified by flash column chromatography over a 4 g silica gel (gradient: DCM:MeOH from 100:0 to 85:15). The desired fractions were collected and the solvent was concentrated under vacuum to give 2-chloro-N-(6-methyl-5-((1-methyl-6-((1-methyl-1H-imidazol-4-yl)amino)-1H-pyrazolo[3,4-d]pyrimidin-3-yl)amino)pyridin-3-yl)acetamide (61 mg, 57%) as a pale brown solid. LCMS (ESI): mass for C₁₈H₁₉ClN₁₀O: 426.9; m/z found: 427.2 [M+H]⁺.

### Step f: 2-(3,3-dimethylazetidin-1-yl)-N-(6-methyl-5-((1-methyl-6-((1-methyl-1H-imidazol-4-yl)amino)-1H-pyrazolo[3,4-d]pyrimidin-3-yl)amino)pyridin-3-yl)acetamide

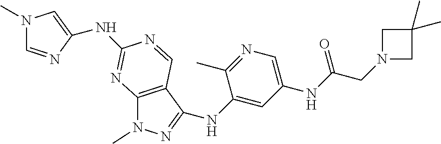

To the mixture of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl) acetamide (60 mg, 0.117 mmol) in DMF (3 mL) was added (S)-3-methylpyrrolidine hydrochloride (44 mg, 0.362 mmol), K₂CO₃ (65 mg, 0.470 mmol), sodium iodide (20 mg, 0.133 mmol). The reaction mixture was stirred at 50° C. for 1.5 hours. The reaction mixture was concentrated under vacuum to give product as a brown solid which was purified by preparative high-performance liquid chromatography over column: Phenomenex C18 80*40 mm*3 um to give the title compound (21 mg, 37%) as a yellow solid. LCMS (ESI): mass for C₂₃H₂₉N₁₁O: 475.5; m/z found: 476.3 [M+H]⁺. ¹H NMR (400 MHz, METHANOL-d₄) δ 8.93-8.82 (m, 1H), 8.71-8.60 (m, 1H), 8.40-8.28 (m, 1H), 7.56-7.45 (m, 1H), 7.44-7.37 (m, 1H), 3.90-3.82 (m, 3H), 3.80-3.74 (m, 3H), 3.43-3.37 (m, 2H), 3.21 (br s, 4H), 2.58-2.50 (m, 3H), 1.31-1.26 (m, 6H).

### Example 142. (S)—N-(6-methyl-5-((1-methyl-6-((1-methyl-1H-imidazol-4-yl)amino)-1H-pyrazolo[3,4-d]pyrimidin-3-yl)amino)pyridin-3-yl)-2-(2-methylpyrrolidin-1-yl)acetamide

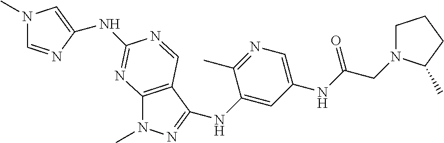 [SMILES: Cc1ncc(NC(=O)CN2CCC[C@@H]2C)cc1Nc1nn(C)c2nc(Nc3cn(C)cn3)ncc12]

To the mixture of 2-chloro-N-(6-methyl-5-((1-methyl-6-((1-methyl-1H-pyrazol-4-yl)amino)-1H-pyrazolo[3,4-d]pyrimidin-3-yl)amino)pyridin-3-yl) acetamide (150 mg, 0.14 mmol) in DMF (3 mL) was added (S)-3-methylpyrrolidine hydrochloride (38 mg, 0.4 mmol), K₂CO₃ (81 mg, 0.6 mmol), sodium iodide (24 mg, 0.2 mmol). The reaction mixture was stirred at 50° C. for 1.5 hours. The reaction mixture was filtered and the filtrate was concentrated under vacuum to give crude product, which was purified by preparative high-performance liquid chromatography over column: Phenomenex C18 80*40 mm*3 um to give the title compound (S)—N-(6-methyl-5-((1-methyl-6-((1-methyl-1H-imidazol-4-yl)amino)-1H-pyrazolo[3,4-d]pyrimidin-3-yl)amino)pyridin-3-yl)-2-(2-methylpyrrolidin-1-yl)acetamide (11.7 mg, 16.7%) as a white solid. LCMS (ESI): mass calcd. for C₂₃H₂₉N₁₁O 475.5; m/z found, 476.3 [M+H]⁺. ¹H NMR (400 MHz, METHANOL-d₄) δ 8.82-8.77 (m, 1H), 8.62-8.57 (m, 1H), 8.30-8.24 (m, 1H), 7.46-7.40 (m, 1H), 7.39-7.31 (m, 1H), 3.79 (s, 3H), 3.70 (s, 3H), 3.51-3.44 (m, 1H), 3.21-3.15 (m, 1H), 3.05-2.97 (m, 1H), 2.55-2.50 (m, 1H), 2.49-2.44 (m, 3H), 2.36-2.28 (m, 1H), 1.98-1.92 (m, 1H), 1.84-1.67 (m, 2H), 1.48-1.38 (m, 1H), 1.13-1.03 (m, 3H)

### Example 143. (S)—N-(6-methyl-5-((1-methyl-6-((1-methyl-1H-pyrazol-5-yl)amino)-1H-pyrazolo[3,4-d]pyrimidin-3-yl)amino)pyridin-3-yl)-2-(2-methylpyrrolidin-1-yl)acetamide

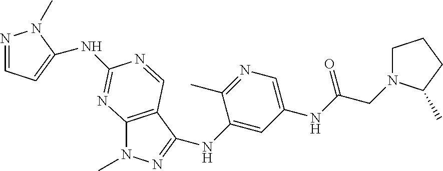 [SMILES: Cc1ncc(NC(=O)CN2CCC[C@@H]2C)cc1Nc1nn(C)c2nc(Nc3ccnn3C)ncc12]

To a solution of 2-chloro-N-(6-methyl-5-((1-methyl-6-((1-methyl-1H-pyrazol-5-yl)amino)-1H-pyrazolo[3,4-d]pyrimidin-3-yl)amino)pyridin-3-yl)acetamide (80 mg, 0.16 mmol), potassium carbonate (64.1 mg, 0.46 mmol), sodium iodide (25.5 mg, 0.17 mmol) in DMF (5 mL) were added (S)-2-methylpyrrolidine (39.5 mg, 0.46 mmol) at room temperature. The reaction was stirred at 50° C. for 2 h. The reaction mixture was concentrated in vacuo to give the crude product, which was purified by preparative high-performance liquid chromatography over column: Boston Green ODS 150*30 mm*5 um to give the title compound (S)—N-(6-methyl-5-((1-methyl-6-((1-methyl-1H-pyrazol-5-yl)amino)-1H-pyrazolo[3,4-d]pyrimidin-3-yl)amino)pyridin-3-yl)-2-(2-methylpyrrolidin-1-yl)acetamide (20 mg, 25%) as a white solid. LCMS (ESI): mass calcd. for C₂₃H₂₉N₁₁O, 475.5; m/z found, 476.4 [M+H]⁺. ¹H NMR (400 MHz, METHANOL-d₄) δ 8.91 (s, 1H), 8.68 (d, J=2.0 Hz, 1H), 8.35 (d, J=2.0 Hz, 1H), 7.47 (d, J=2.0 Hz, 1H), 6.42 (d, J=2.0 Hz, 1H), 3.79 (d, J=4.0 Hz, 6H), 3.56 (d, J=16.1 Hz, 1H), 3.27 (dt, J=3.3, 8.7 Hz, 1H), 3.11 (d, J=15.8 Hz, 1H), 2.64-2.59 (m, 1H), 2.57 (s, 3H), 2.41 (q, J=8.8 Hz, 1H), 2.07-1.98 (m, 1H), 1.92-1.73 (m, 2H), 1.59-1.46 (m, 1H), 1.17 (d, J=6.0 Hz, 3H)

### Example 144. (S)—N-(6-methyl-5-((1-methyl-6-((1-(oxetan-3-yl)-1H-pyrazol-4-yl)amino)-1H-pyrazolo[3,4-d]pyrimidin-3-yl)amino)pyridin-3-yl)-2-(2-methylpyrrolidin-1-yl)acetamide

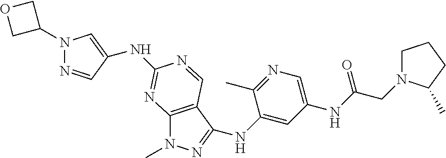 [SMILES: Cc1ncc(NC(=O)CN2CCC[C@@H]2C)cc1Nc1nn(C)c2nc(Nc3cnn(C4COC4)c3)ncc12]

To the mixture of 2-chloro-N-(6-methyl-5-((1-methyl-6-((1-(oxetan-3-yl)-1H-pyrazol-4-yl)amino)-1H-pyrazolo[3,4-d]pyrimidin-3-yl)amino)pyridin-3-yl)acetamide (50 mg, 0.10 mmol) in DMF (2 mL) was added (S)-2-methylpyrrolidine (10 mg, 0.12 mmol), K₂CO₃ (35 mg, 0.25 mmol) and sodium iodide (7 mg, 0.05 mmol) at room temperature. The resulting mixture was heated at 50° C. for 2 hours before cooling to room-temperature. The reaction mixture was filtered and the filtrate was concentrated under vacuum to give crude product, which was purified by preparative high-performance liquid chromatography over column: Phenomenex C18 80*40 mm*3 um to give the title compound (S)—N-(6-methyl-5-((1-methyl-6-((1-(oxetan-3-yl)-1H-pyrazol-4-yl)amino)-1H-pyrazolo[3,4-d]pyrimidin-3-yl)amino)pyridin-3-yl)-2-(2-methylpyrrolidin-1-yl)acetamide (11 mg, 22%) as a yellow solid. LCMS (ESI): mass calcd. For C₂₅H₃₁N₁₁O₂, 517.6; m/z found, 518.3 [M+H]⁺. ¹H NMR (400 MHz, METHANOL-d₄) δ ppm 8.78 (s, 1H), 8.58 (d, J=2.15 Hz, 1H), 8.28 (d, J=2.27 Hz, 1H), 8.21 (s, 1H), 7.75 (s, 1H), 5.51 (quin, J=6.88 Hz, 1H), 4.93-5.14 (m, 4H), 3.78 (s, 3H), 3.40 (br d, J=10.13 Hz, 2H), 2.49 (s, 3H), 2.05 (br s, 1H), 1.76-1.99 (m, 2H), 1.54 (br s, 1H), 1.26 (br s, 1H), 1.22 (s, 3H), 1.19 (br d, J=6.08 Hz, 2H)

### Example 145. (S)—N-(6-methyl-5-((1-methyl-6-((2-methylpyridin-3-yl)amino)-1H-pyrazolo[3,4-d]pyrimidin-3-yl)amino)pyridin-3-yl)-2-(2-methylpyrrolidin-1-yl)acetamide

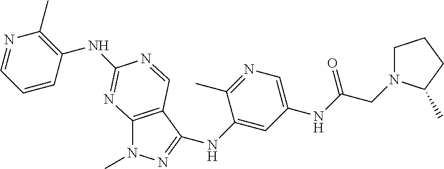 [SMILES: Cc1ncccc1Nc1ncc2c(Nc3cc(NC(=O)CN4CCC[C@@H]4C)cnc3C)nn(C)c2n1]

### Step a: methyl 6-methyl-5-((1-methyl-6-((2-methylpyridin-3-yl)amino)-1H-pyrazolo[3,4-d]pyrimidin-3-yl)amino)nicotinate

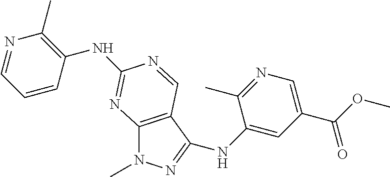 [SMILES: COC(=O)c1cnc(C)c(Nc2nn(C)c3nc(Nc4cccnc4C)ncc23)c1]

To a mixture of ethyl 5-((6-chloro-1-methyl-1H-pyrazolo[3,4-d]pyrimidin-3-yl)amino)-6-methylnicotinate (1.0 g, 3.0 mmol), 2-methylpyridin-3-amine (0.39 g, 3.6 mmol), Brettphos-Pd-G3 (0.55 g, 0.61 mmol) and Brettphos (0.33 g, 0.62 mmol) in DMF (50 mL) was added Cs₂CO₃ (2.9 g, 9.0 mmol) at room temperature under N₂ atmosphere. Then the reaction mixture was purged with N₂ for 2 minutes. The reaction mixture was stirred at 90° C. for 15 hrs. The reaction mixture was filtered after cooling down room temperature. The filtrate was concentrated in vacuo to give crude of methyl 6-methyl-5-((1-methyl-6-((2-methylpyridin-3-yl)amino)-1H-pyrazolo[3,4-d]pyrimidin-3-yl)amino)nicotinate (2.5 g, crude) as brown oil which was used in the next step without further work up and purification. LCMS (ESI): mass calcd. for C₂₀H₂₀N₈O₂, 404.2; m/z found, 405.3 [M+H]⁺.

### Step b: 6-methyl-5-((1-methyl-6-((2-methylpyridin-3-yl)amino)-1H-pyrazolo[3,4-d]pyrimidin-3-yl)amino)nicotinic acid

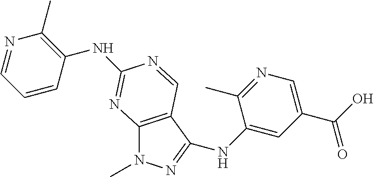 [SMILES: Cc1ncccc1Nc1ncc2c(Nc3cc(C(=O)O)cnc3C)nn(C)c2n1]

To a solution of methyl 6-methyl-5-((1-methyl-6-((2-methylpyridin-3-yl)amino)-1H-pyrazolo[3,4-d]pyrimidin-3-yl)amino)nicotinate (1.4 g, 3.5 mmol) in THF (10 mL) was added aq LiOH (10 mL, 1M, 10 mmol). The reaction was stirred at room temperature for 3 hrs. The reaction mixture was concentrated to remove solvent. The mixture was adjusted to pH-5 with 1N HCl and brown precipitate out. The mixture was filtered. The brown solid was triturated with EtOAc (20 mL). The mixture was filtered. Then the brown solid was dried in vacuo to give 6-methyl-5-((1-methyl-6-((2-methylpyridin-3-yl)amino)-1H-pyrazolo[3,4-d]pyrimidin-3-yl)amino)nicotinic acid (0.91 g, 67%) which was used in the next step without further work up and purification. LCMS (ESI): mass calcd. for C₁₉H₁₈N₈O₂, 390.2; m/z found, 391.1 [M+H]⁺.

### Step c: tert-butyl (6-methyl-5-((1-methyl-6-((2-methylpyridin-3-yl)amino)-1H-pyrazolo[3,4-d]pyrimidin-3-yl)amino)pyridin-3-yl)carbamate

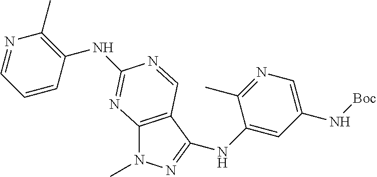 [SMILES: Cc1ncccc1Nc1ncc2c(Nc3cc(NC(=O)OC(C)(C)C)cnc3C)nn(C)c2n1]

To a solution of 6-methyl-5-((1-methyl-6-((2-methylpyridin-3-yl)amino)-1H-pyrazolo[3,4-d]pyrimidin-3-yl)amino)nicotinic acid (856 mg, 2.2 mmol) in t-BuOH (90 mL) was added TEA (450 mg, 4.4 mmol) and DPPA (910 mg, 3.3 mmol) at room temperature. The mixture was heated to 100° C. and stirred for 15 hrs. The reaction mixture was concentrated in vacuo to give brown oil. The brown oil was purified by column chromatography over silica gel (eluent: dichloromethane/methanol=100:0 to 90:10). The desired fractions were collected and the solvent was concentrated under vacuum to give desired product tert-butyl (6-methyl-5-((1-methyl-6-((2-methylpyridin-3-yl)amino)-1H-pyrazolo[3,4-d]pyrimidin-3-yl)amino)pyridin-3-yl)carbamate (1.1 g, 72%) as a pale brown solid. LCMS (ESI): mass calcd. for C₂₃H₂₇N₉O₂, 461.2; m/z found, 462.1 [M+H]⁺.

### Step d: N³-(5-amino-2-methylpyridin-3-yl)-1-methyl-N⁶-(2-methylpyridin-3-yl)-1H-pyrazolo [3,4-d]pyrimidine-3,6-diamine

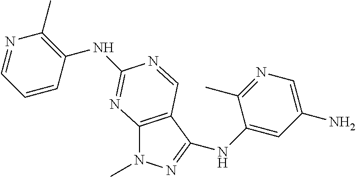 [SMILES: Cc1ncccc1Nc1ncc2c(Nc3cc(N)cnc3C)nn(C)c2n1]

To a solution of tert-butyl (6-methyl-5-((1-methyl-6-((2-methylpyridin-3-yl)amino)-1H-pyrazolo[3,4-d]pyrimidin-3-yl)amino)pyridin-3-yl)carbamate (1.1 g, 1.6 mmol) in DCM (5.0 mL) was added HCl/dioxane (4.0 mL, 16 mmol, 4M) at room temperature. The reaction mixture was stirred at room temperature for 15 hrs. The reaction mixture was concentrated in vacuo to give yellow gum. The yellow gum was triturated with mixture of DCM and MeOH (1:1, 20 mL). The mixture was filtered. The filter cake was washed with DCM. The filer cake was dried in vacuo to give N³-(5-amino-2-methylpyridin-3-yl)-1-methyl-N⁶-(2-methylpyridin-3-yl)-1H-pyrazolo[3,4-d]pyrimidine-3,6-diamine (75 mg, 12%) as a yellow solid. LCMS (ESI): mass calcd. for C₁₈H₁₉N₉, 361.2; m/z found, 362.1 [M+H]⁺.

### Step e: 2-chloro-N-(6-methyl-5-((1-methyl-6-((2-methylpyridin-3-yl)amino)-1H-pyrazolo[3,4-d]pyrimidin-3-yl)amino)pyridin-3-yl)acetamide

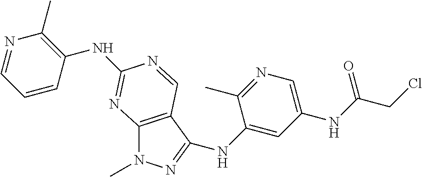 [SMILES: Cc1ncccc1Nc1ncc2c(Nc3cc(NC(=O)CCl)cnc3C)nn(C)c2n1]

To a mixture of N³-(5-amino-2-methylpyridin-3-yl)-1-methyl-N⁶-(2-methylpyridin-3-yl)-1H-pyrazolo[3,4-d]pyrimidine-3,6-diamine (75 mg, 0.19 mmol) and NaHCO₃ (55 mg, 0.66 mmol) in DMF (3 mL) was added 2-chloroacetyl chloride (25 mg, 0.22 mmoL) dropwise at 0° C. Then the reaction was stirred at room temperature for 1.5 hrs. The reaction mixture was filtered. The filtrate was concentrated in vacuo to give crude which was purified by column chromatography over silica gel (eluent: dichloromethane/methanol=100:0 to 85:15). The desired fractions were collected, and the solvent was concentrated under vacuum to give desired product 2-chloro-N-(6-methyl-5-((1-methyl-6-((2-methylpyridin-3-yl)amino)-1H-pyrazolo[3,4-d]pyrimidin-3-yl)amino)pyridin-3-yl)acetamide (77 mg, 93%) as a pale brown solid. LCMS (ESI): mass calcd. for C₂₀H₂₀ClN₉O, 437.1; m/z found, 438.2 [M+H]⁺.

### Step f: (S)—N-(6-methyl-5-((1-methyl-6-((2-methylpyridin-3-yl)amino)-1H-pyrazolo[3,4-d]pyrimidin-3-yl)amino)pyridin-3-yl)-2-(2-methylpyrrolidin-1-yl)acetamide

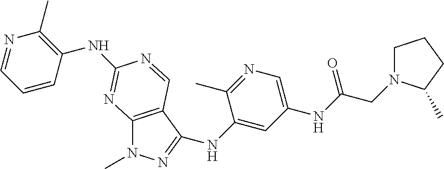 [SMILES: Cc1ncccc1Nc1ncc2c(Nc3cc(NC(=O)CN4CCC[C@@H]4C)cnc3C)nn(C)c2n1]

To a solution of 2-chloro-N-(6-methyl-5-((1-methyl-6-((2-methylpyridin-3-yl)amino)-1H-pyrazolo[3,4-d]pyrimidin-3-yl)amino)pyridin-3-yl)acetamide (77 mg, 0.18 mmol) and (S)-2-methylpyrrolidine (45 mg, 0.53 mmol) in DMF (3 mL) was added K₂CO₃ (73 mg, 0.53 mmol) and sodium iodide (30 mg, 0.20 mmol). The reaction mixture was stirred at 50° C. for 1.5 hrs. The reaction mixture was purified by preparative high-performance liquid chromatography over column: Phenomenex C18 80*40 mm*3 um to give the title compound (S)—N-(6-methyl-5-((1-methyl-6-((2-methylpyridin-3-yl)amino)-1H-pyrazolo[3,4-d]pyrimidin-3-yl)amino)pyridin-3-yl)-2-(2-methylpyrrolidin-1-yl)acetamide (41 mg, 46%) as an off-white solid. LCMS (ESI): mass calcd. for C₂₅H₃₀N₁₀O, 486.3; m/z found, 487.3 [M+H]⁺. ¹H NMR (400 MHz, METHANOL-d₄) δ 8.82 (s, 1H), 8.60 (d, J=2.1 Hz, 1H), 8.26 (d, J=2.1 Hz, 1H), 8.23 (dd, J=1.4, 8.1 Hz, 1H), 8.14 (dd, J=1.4, 4.9 Hz, 1H), 7.25 (dd, J=4.9, 8.0 Hz, 1H), 3.68 (s, 3H), 3.47 (d, J=16.0 Hz, 1H), 3.18 (dt, J=3.2, 8.6 Hz, 1H), 3.02 (d, J=16.0 Hz, 1H), 2.56-2.49 (m, 1H), 2.48 (d, J=3.3 Hz, 6H), 2.32 (q, J=8.9 Hz, 1H), 2.00-1.89 (m, 1H), 1.85-1.66 (m, 2H), 1.50-1.37 (m, 1H), 1.08 (d, J=6.1 Hz, 3H).

### Example 146. (S)—N-(5-((6-((2-methoxypyridin-3-yl)amino)-1-methyl-1H-pyrazolo[3,4-d]pyrimidin-3-yl)amino)-6-methylpyridin-3-yl)-2-(2-methylpyrrolidin-1-yl)acetamide

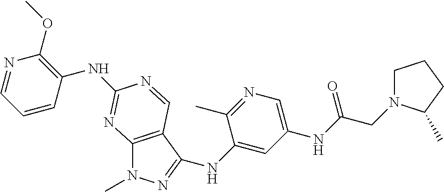 [SMILES: COc1ncccc1Nc1ncc2c(Nc3cc(NC(=O)CN4CCC[C@@H]4C)cnc3C)nn(C)c2n1]

### Step a: methyl 5-((6-((2-methoxypyridin-3-yl)amino)-1-methyl-1H-pyrazolo[3,4-d]pyrimidin-3-yl)amino)-6-methylnicotinate

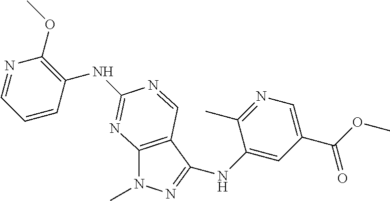 [SMILES: COC(=O)c1cnc(C)c(Nc2nn(C)c3nc(Nc4cccnc4OC)ncc23)c1]

To a solution of methyl 5-((6-chloro-1-methyl-1H-pyrazolo[3,4-d]pyrimidin-3-yl)amino)-6-methylnicotinate (500 mg, 1.5 mmol), 4 A molecular sieve (500 mg), 2-methoxypyridin-3-amine (280 mg, 2.3 mmol) and Cs₂CO₃ (1.5 g, 4.6 mmol) in DMF (10 mL) was added Brettphos (167 mg, 0.31 mmol) and Brettphos Pd G₃ (275 mg, 0.30 mmol). The reaction mixture was heated at 100° C. under N₂ overnight. The reaction mixture was cooled to room temperature. The reaction mixture was filtered through a pad of celite and the pad was washed with DCM (5 mL×3). The combined filtrates were concentrated to dryness to give a black solid. The black solid was triturated with ethyl acetate (5 mL) at room temperature for 30 min., then filtered. The solid was rinsed with 2 mL ethyl acetate, collected and dried to dryness in vacuo to give the title compound methyl 5-((6-((2-methoxypyridin-3-yl)amino)-1-methyl-1H-pyrazolo[3,4-d]pyrimidin-3-yl)amino)-6-methylnicotinate (1.1 g, 60%) as a yellow solid. LCMS (ESI): mass calcd. For C₂₀H₂₀N₈O₃, 420.1; m/z found, 421.3 [M+H]⁺.

### Step b: 5-((6-((2-methoxypyridin-3-yl)amino)-1-methyl-1H-pyrazolo[3,4-d]pyrimidin-3-yl)amino)-6-methylnicotinic acid

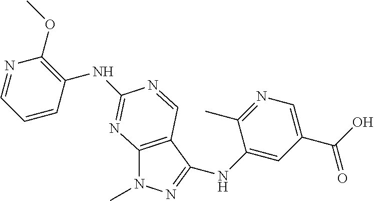 [SMILES: COc1ncccc1Nc1ncc2c(Nc3cc(C(=O)O)cnc3C)nn(C)c2n1]

To a solution of methyl 5-((6-((2-methoxypyridin-3-yl)amino)-1-methyl-1H-pyrazolo[3,4-d]pyrimidin-3-yl)amino)-6-methylnicotinate (1.1 g, 1.3 mmol) in THE (27 mL) and H₂O (9 mL) was added LiOH—H₂O (180 mg, 4.3 mmol) dropwise via syringe at room temperature over 1 min. The mixture was stirred at room temperature for 1 hour. The reaction mixture was poured into 10 mL of ice-water carefully and acidified with 1N HCl to pH=5. The mixture was filtered, rinsed with H₂O (5 mL). The filter cake was dried in vacuo to afford 5-((6-((2-methoxypyridin-3-yl)amino)-1-methyl-1H-pyrazolo[3,4-d]pyrimidin-3-yl)amino)-6-methylnicotinic acid (520 mg, 96%) as a brown solid. LCMS (ESI): mass calcd. for C₁₉H₁₈N₈O₃, 406.4; m/z found, 407.0[M+H]⁺.

### Step c: tert-butyl (5-((6-((2-methoxypyridin-3-yl)amino)-1-methyl-1H-pyrazolo[3,4-d]pyrimidin-3-yl)amino)-6-methylpyridin-3-yl)carbamate

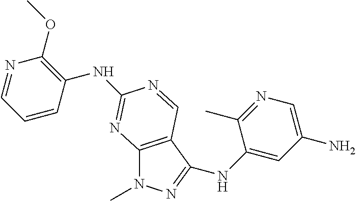 [SMILES: COc1ncccc1Nc1ncc2c(Nc3cc(N)cnc3C)nn(C)c2n1]

To a solution of 5-((6-((2-methoxypyridin-3-yl)amino)-1-methyl-1H-pyrazolo[3,4-d]pyrimidin-3-yl)amino)-6-methylnicotinic acid (520 mg, 1.2 mmol) in t-BuOH (10 mL) was added TEA (0.34 mL, 2.5 mmol) and DPPA (0.53 mL, 2.5 mmol) at room temperature. The mixture was heated to 100° C. and stirred overnight. The reaction mixture was poured into 5 mL of ice-water. The mixture was filtered, rinsed with H₂O (2 mL). The filter cake was dried in vacuo to afford a brown solid. The brown solid was subjected to column chromatography over silica gel (gradient elution: 0-20% methanol in dichloromethane). The pure fractions were collected and concentrated to dryness in vacuum to give tert-butyl (5-((6-((2-methoxypyridin-3-yl)amino)-1-methyl-1H-pyrazolo[3,4-d]pyrimidin-3-yl)amino)-6-methylpyridin-3-yl)carbamate (200 mg, 25%) as black solid. LCMS (ESI): mass calcd. for C₂₃H₂₇N₉O₃, 477.5; m/z found, 478.3 [M+H]⁺.

### Step d: N³-(5-amino-2-methylpyridin-3-yl)-N⁶-(2-methoxypyridin-3-yl)-1-methyl-1H-pyrazolo [3,4-d]pyrimidine-3,6-diamine

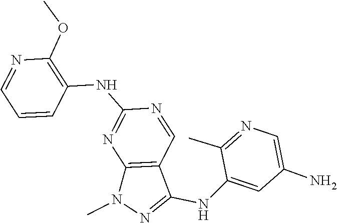 [SMILES: COc1ncccc1Nc1ncc2c(Nc3cc(N)cnc3C)nn(C)c2n1]

To the mixture of tert-butyl (5-((6-((2-methoxypyridin-3-yl)amino)-1-methyl-1H-pyrazolo[3,4-d]pyrimidin-3-yl)amino)-6-methylpyridin-3-yl)carbamate (170 mg, 0.27 mmol) in DCM (2 mL) was added TFA (0.2 mL, 2.7 mmol). The resulting mixture was stirred at room temperature for 1 hour. The reaction mixture was quenched with 2 mL of sat. aq. NaHCO₃ at 20° C. The mixture was filtered and the solid was rinsed with 2 mL water, collected and dried to dryness in vacuo to give N³-(5-amino-2-methylpyridin-3-yl)-N⁶-(2-methoxypyridin-3-yl)-1-methyl-1H-pyrazolo[3,4-d]pyrimidine-3,6-diamine (105 mg, 88%) as yellow solid. LCMS (ESI): mass calcd. for C₁₈H₁₉N₉O, 337.4; m/z found, 378.0 [M+H]⁺.

### Step e: 2-chloro-N-(5-((6-((2-methoxypyridin-3-yl)amino)-1-methyl-1H-pyrazolo[3,4-d]pyrimidin-3-yl)amino)-6-methylpyridin-3-yl)acetamide

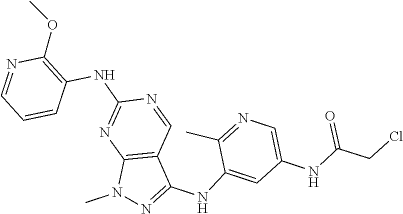 [SMILES: COc1ncccc1Nc1ncc2c(Nc3cc(NC(=O)CCl)cnc3C)nn(C)c2n1]

To a solution of N³-(5-amino-2-methylpyridin-3-yl)-N⁶-(2-methoxypyridin-3-yl)-1-methyl-1H-pyrazolo[3,4-d]pyrimidine-3,6-diamine (100 mg, 0.22 mmol) in DCM (5 mL) was added NaHCO₃ (200 mg, 2.4 mmol) and 2-chloroacetyl chloride (0.1 mL, 1.2 mmol). The mixture was stirred at room temperature for 1 hour. The reaction mixture was quenched with 2 mL of sat. aq. NaHCO₃ at 20° C. The mixture was filtered and the solid was rinsed with 1 mL water, collected and dried to dryness in vacuo to give 2-chloro-N-(5-((6-((2-methoxypyridin-3-yl)amino)-1-methyl-1H-pyrazolo[3,4-d]pyrimidin-3-yl)amino)-6-methylpyridin-3-yl)acetamide (100 mg, 82%) as a yellow solid. LCMS (ESI): mass calcd. for C₂₀H₂₀ClN₉O₂, 453.8; m/z found, 454.2 [M+H]⁺.

### Step f: (S)—N-(5-((6-((2-methoxypyridin-3-yl)amino)-1-methyl-1H-pyrazolo[3,4-d]pyrimidin-3-yl)amino)-6-methylpyridin-3-yl)-2-(2-methylpyrrolidin-1-yl)acetamide

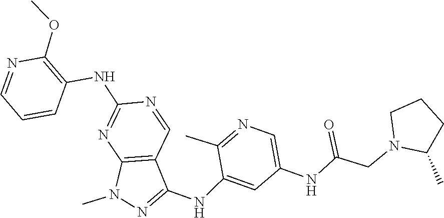 [SMILES: COc1ncccc1Nc1ncc2c(Nc3cc(NC(=O)CN4CCC[C@@H]4C)cnc3C)nn(C)c2n1]

To a solution of 2-chloro-N-(5-((6-((2-methoxypyridin-3-yl)amino)-1-methyl-1H-pyrazolo[3,4-d]pyrimidin-3-yl)amino)-6-methylpyridin-3-yl)acetamide (95 mg, 0.17 mmol) in DMF (2 mL) was added (S)-2-methylpyrrolidine (30 mg, 0.35 mmol), K₂CO₃ (75 mg, 0.54 mmol) and sodium iodide (16 mg, 0.11 mmol) at room temperature. The reaction mixture was stirred at 50° C. for 1 hour. The reaction mixture was cooled to room temperature. The reaction mixture was filtered through a pad of celite and the pad was washed with DCM (2 mL×3). The combined filtrates were concentrated under vacuum to give crude product, which was purified by preparative high-performance liquid chromatography over column: Phenomenex C18 80*40 mm*3 um to give the title compound. (S)—N-(5-((6-((2-methoxypyridin-3-yl)amino)-1-methyl-1H-pyrazolo[3,4-d]pyrimidin-3-yl)amino)-6-methylpyridin-3-yl)-2-(2-methylpyrrolidin-1-yl)acetamide (48.6 mg, 56%) as yellow powder. LCMS (ESI): mass calcd. for C₂₅H₃₀N₁₀O₂, 502.5; m/z found, 503.3 [M+H]⁺.

¹H NMR (400 MHz, METHANOL-d₄) δ ppm 8.91 (s, 1H), 8.89 (d, J=7.36 Hz, 1H), 8.73 (d, J=2.15 Hz, 1H), 8.32 (d, J=2.15 Hz, 1H), 7.76 (dd, J=5.01, 1.43 Hz, 1H), 7.00 (dd, J=7.81, 5.07 Hz, 1H), 4.06 (s, 3H), 3.85 (s, 3H), 3.47-3.63 (m, 1H), 3.27 (br dd, J=8.64, 5.66 Hz, 1H), 3.11 (d, J=15.85 Hz, 1H), 2.59-2.65 (m, 1H), 2.55 (s, 3H), 2.41 (d, J=8.82 Hz, 1H), 2.02 (br s, 1H), 1.80 (br s, 2H), 1.52 (br s, 1H), 1.18 (d, J=6.08 Hz, 3H).

### Example 147. N-(2-(5-azaspiro[3.4]octan-5-yl)ethyl)-5-((6-((1-(2-methoxyethyl)-1H-pyrazol-4-yl)amino)-1-methyl-1H-pyrazolo[3,4-d]pyrimidin-3-yl)amino)-6-methylnicotinamide

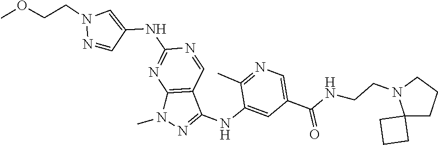 [SMILES: COCCn1cc(Nc2ncc3c(Nc4cc(C(=O)NCCN5CCCC56CCC6)cnc4C)nn(C)c3n2)cn1]

To the mixture of 5-((6-((1-(2-methoxyethyl)-1H-pyrazol-4-yl)amino)-1-methyl-1H-pyrazolo[3,4-d]pyrimidin-3-yl)amino)-6-methylnicotinic acid (130 mg, 0.31 mmol) in DMF (5 mL) was added 2-(5-azaspiro[3.4]octan-5-yl)ethanamine (65 mg, 0.42 mmol) and DIEA (210 μL, 1.3 mmol), then HATU (143 mg, 0.38 mmol) was added. The mixture stirred at 25° C. for 1 h and then concentrated under vacuum to give the crude product, which was purified by preparative high-performance liquid chromatography over column: Phenomenex C18 80*40 mm*3 um to give the title compound N-(2-(5-azaspiro[3.4]octan-5-yl)ethyl)-5-((6-((1-(2-methoxyethyl)-1H-pyrazol-4-yl)amino)-1-methyl-1H-pyrazolo[3,4-d]pyrimidin-3-yl)amino)-6-methylnicotinamide (32 mg, 19%) as white solid. LCMS (ESI): mass calcd. for C₂₈H₃₇N₁O₂, 559.7; m/z found, 560.4 [M+H]⁺. ¹H NMR (400 MHz, METHANOL-d₄) δ ppm 8.88 (s, 1H), 8.77 (d, J=1.91 Hz, 1H), 8.49 (s, 1H), 8.19 (br s, 1H), 7.71 (s, 1H), 4.33 (t, J=5.13 Hz, 2H), 3.88 (s, 3H), 3.79 (t, J=5.19 Hz, 2H), 3.52-3.62 (m, 2H), 3.37 (s, 3H), 2.88 (br s, 4H), 2.67 (s, 3H), 2.23-2.38 (m, 2H), 1.95-2.12 (m, 2H), 1.85 (quin, J=7.54 Hz, 2H), 1.65-1.80 (m, 4H)

### Example 148. N-(2-(2,2-dimethylpyrrolidin-1-yl)ethyl)-5-((6-((1-(2-methoxyethyl)-1H-pyrazol-4-yl)amino)-1-methyl-1H-pyrazolo[3,4-d]pyrimidin-3-yl)amino)-6-methylnicotinamide

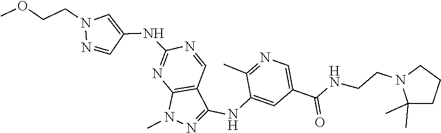 [SMILES: COCCn1cc(Nc2ncc3c(Nc4cc(C(=O)NCCN5CCCC5(C)C)cnc4C)nn(C)c3n2)cn1]

To a solution of 2-(2,2-dimethylpyrrolidin-1-yl)ethanamine (50 mg, 0.337 mmol) in DMF (3 mL) was added 5-((6-((1-(2-methoxyethyl)-1H-pyrazol-4-yl)amino)-1-methyl-1H-pyrazolo[3,4-d]pyrimidin-3-yl)amino)-6-methylnicotinic acid (100 mg, 0.233 mmol), DIEA (162 μL, 0.980 mmol) and HATU (110 mg, 0.289 mmol). The resulting mixture was stirred at room temperature for 2 h. The reaction mixture was concentrated under vacuum to give product as brown solid which was purified by preparative high-performance liquid chromatography over column: Xtimate C18 150*40 mm*5 um to give the title compound (45.6 mg, 31%) as a yellow solid. LCMS (ESI): mass for C₂₇H₃₇N₁O₂: 547.7; m/z found: 548.4 [M+H]⁺. ¹H NMR (400 MHz, CD₃OD) δ 9.46-9.33 (m, 1H), 9.08-8.96 (m, 1H), 8.65-8.49 (m, 1H), 8.26-8.08 (m, 1H), 7.83-7.66 (m, 1H), 4.39-4.31 (m, 2H), 3.99-3.90 (m, 4H), 3.90-3.83 (m, 1H), 3.82-3.71 (m, 3H), 3.58-3.49 (m, 1H), 3.42-3.36 (m, 4H), 3.25-3.12 (m, 1H), 2.88-2.83 (m, 3H), 2.27-1.95 (m, 4H), 1.56-1.50 (m, 3H), 1.37-1.32 (m, 3H).

### Example 149. N-(2-(1-azaspiro[3.3]heptan-1-yl)ethyl)-6-methyl-5-((1-methyl-6-((1-methyl-1H-pyrazol-4-yl)amino)-1H-pyrazolo [3,4-d]pyrimidin-3-yl)amino)nicotinamide

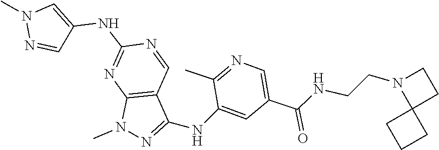 [SMILES: Cc1ncc(C(=O)NCCN2CCC23CCC3)cc1Nc1nn(C)c2nc(Nc3cnn(C)c3)ncc12]

To the mixture of 6-methyl-5-((1-methyl-6-((1-methyl-1H-pyrazol-4-yl)amino)-1H-pyrazolo[3,4-d]pyrimidin-3-yl)amino)nicotinic acid (170 mg, 0.42 mmol) in DMF (10 mL) was added 2-(1-azaspiro[3.3]heptan-1-yl)ethanamine (85 mg, 0.61 mmol) and DIEA (300 μL, 1.8 mmol), then HATU (204 mg, 0.54 mmol) was added. The mixture stirred at 25° C. for 1 h and then concentrated under vacuum to give the crude product, which was purified by preparative high-performance liquid chromatography over column: Boston Prime C18 150*30 mm*5 um to give the title compound N-(2-(1-azaspiro[3.3]heptan-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (31 mg, 14%) as white solid. LCMS (ESI): mass calcd. for C₂₅H₃₁N₁₁O, 501.6; m/z found, 502.3 [M+H]⁺. ¹H NMR (400 MHz, METHANOL-d₄) δ ppm 8.84-8.91 (m, 1H), 8.77 (d, J=1.91 Hz, 1H), 8.42-8.52 (m, 1H), 8.11 (br s, 1H), 7.67 (s, 1H), 3.93 (d, J=1.79 Hz, 3H), 3.84-3.90 (m, 3H), 3.41-3.53 (m, 2H), 3.24 (br t, J=7.03 Hz, 2H), 2.71-2.82 (m, 2H), 2.67 (d, J=2.74 Hz, 3H), 2.32-2.43 (m, 2H), 2.22-2.30 (m, 2H), 1.99 (br dd, J=6.62, 3.16 Hz, 2H), 1.62-1.74 (m, 2H)

### Example 150. N-(2-(4-azaspiro[2.5]octan-4-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

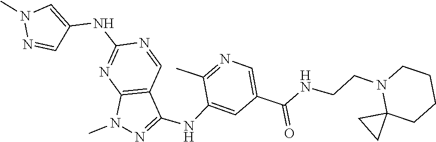 [SMILES: Cc1ncc(C(=O)NCCN2CCCCC23CC3)cc1Nc1nn(C)c2nc(Nc3cnn(C)c3)ncc12]

### Step a: 2-(4-azaspiro[2.5]octan-4-yl)acetonitrile

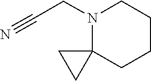 [SMILES: N#CCN1CCCCC12CC2]

To the mixture of 4-azaspiro [2.5]octane (300 mg, 2.698 mmol) in MTBE (12 mL) was added K₂CO₃ (1.1 g, 8 mmol) and 2-bromoacetonitrile (0.2 mL, 2.8 mmol) at room temperature. The reaction was stirred as 25° C. for 12 h. The suspension was filtered through a pad of celite and the pad or filter cake was washed with ethyl acetate (25 mL). The filtrate was concentrated under reduced pressure to give crude product, which was purified by column chromatography over silica gel (eluent: petroleum ether:ethyl acetate from 100:0 to 90:10) to give the title compound 2-(4-azaspiro[2.5]octan-4-yl)acetonitrile (160 mg, 39%) as a pale yellow oil. LCMS (ESI): mass calcd. for C₉H₁₄N₂, 150.2; m/z found, 151.3[M+H]⁺. ¹H NMR (400 MHz, CHLOROFORM-d) δ 3.57 (s, 2H), 2.99-2.91 (m, 2H), 1.64 (quin, J=5.9 Hz, 2H), 1.52-1.44 (m, 2H), 1.34-1.25 (m, 2H), 0.66-0.59 (m, 2H), 0.40-0.30 (m, 2H)

### Step b: 2-(4-azaspiro[2.5]octan-4-yl)ethanamine

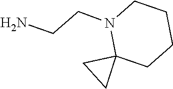 [SMILES: NCCN1CCCCC12CC2]

A mixture of 2-(4-azaspiro[2.5]octan-4-yl)acetonitrile (160 mg, 1.1 mmol) and THF (15 mL) were under N₂ atmosphere. The reaction mixture was cooled to 0° C. via an ice water bath and once cooled for ˜10 mins, LiAlH₄ (60 mg, 1.6 mmol) was added portion wise. After the reaction mixture was cooled to 0° C., the reaction mixture was quenched by addition of 0.1 mL of H₂O, followed by 0.1 mL of 15% aq. NaOH. The reaction mixture was added Mg₂SO₄ (100 mg) stirred at room temperature for 10 min, the solid was removed by filtration. The reaction mixture was filtered. And the filtrate was concentrated to dryness under reduced pressure to afford the crude product 2-(4-azaspiro[2.5]octan-4-yl)ethanamine (120 mg, 73%) as a yellow oil. LCMS (ESI): mass calcd. for C₉H₁₈N₂, 154.2; m/z found, 155.3 [M+H]⁺. ¹H NMR (400 MHz, CHLOROFORM-d) δ 2.77-2.73 (m, 2H), 2.72-2.66 (m, 2H), 2.60-2.55 (m, 2H), 1.63-1.58 (m, 2H), 1.41-1.34 (m, 2H), 1.28-1.15 (m, 2H), 0.50-0.44 (m, 2H), 0.28-0.22 (m, 2H)

### Step c: N-(2-(4-azaspiro[2.5]octan-4-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

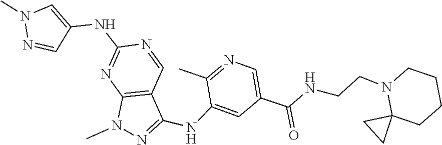 [SMILES: Cc1ncc(C(=O)NCCN2CCCCC23CC3)cc1Nc1nn(C)c2nc(Nc3cnn(C)c3)ncc12]

To the mixture of 6-methyl-5-((1-methyl-6-((1-methyl-1H-pyrazol-4-yl)amino)-1H-pyrazolo[3,4-d]pyrimidin-3-yl)amino)nicotinic acid (170 mg, 0.4 mmol) in DMF (15 mL) was added 2-(4-azaspiro[2.5]octan-4-yl)ethanamine (102 mg, 0.6 mmol), DIEA (0.5 mL, 3 mmol) and HATU (340 mg, 0.9 mmol). The resulting mixture was stirred at room temperature for 2 hours. Which was purified by preparative high-performance liquid chromatography over column: Phenomenex C18 80*40 mm*3 um to give the title compound N-(2-(4-azaspiro[2.5]octan-4-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (62.8 mg, 28%) as a white solid. LCMS (ESI): mass calcd. for C₂₆H₃₃N₁₁O, 515.6; m/z found, 516.3 [M+H]⁺. ¹H NMR (400 MHz, DMSO-d₆) δ 9.77 (br s, 1H), 8.94 (s, 1H), 8.62 (s, 1H), 8.46 (s, 1H), 8.38 (br s, 2H), 8.09 (br s, 1H), 7.56 (s, 1H), 3.83 (br s, 6H), 3.21 (br s, 2H), 2.80 (br s, 4H), 2.60 (s, 3H), 1.62 (br s, 2H), 1.44 (br s, 2H), 1.27 (br s, 2H), 0.45 (br s, 2H), 0.30 (br s, 2H)

### Example 151. N-(2-(5-azaspiro[3.4]octan-5-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

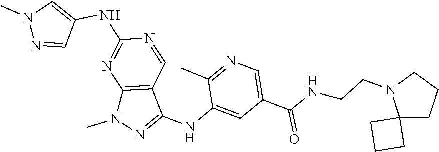 [SMILES: Cc1ncc(C(=O)NCCN2CCCC23CCC3)cc1Nc1nn(C)c2nc(Nc3cnn(C)c3)ncc12]

To the mixture of 6-methyl-5-((1-methyl-6-((1-methyl-1H-pyrazol-4-yl)amino)-1H-pyrazolo[3,4-d]pyrimidin-3-yl)amino)nicotinic acid (170 mg, 0.42 mmol) in DMF (10 mL) was added 2-(5-azaspiro[3.4]octan-5-yl)ethanamine (85 mg, 0.55 mmol) and DIEA (0.3 mL, 1.8 mmol), then HATU (204 mg, 0.54 mmol) was added. The resulting mixture was heated at room temperature for 2 hours and then concentrated under vacuum to give the crude product, which was purified by preparative high-performance liquid chromatography over column: Xtimate C18 150*40 mm*5 um to give the title compound N-(2-(5-azaspiro[3.4]octan-5-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (68.4 mg, 27%) as a yellow solid. LCMS (ESI): mass calcd. for C₂₆H₃₃N₁₁O, 515.3; m/z found, 516.3 [M+H]⁺. 1H NMR (400 MHz, METHANOL-d₄) δ 8.87 (s, 1H), 8.83-8.77 (m, 1H), 8.50 (d, J=1.9 Hz, 1H), 8.48-8.41 (m, 1H), 8.15-8.02 (m, 1H), 7.72-7.63 (m, 1H), 3.92 (s, 3H), 3.86 (s, 3H), 3.76 (t, J=6.3 Hz, 2H), 3.42-3.37 (m, 2H), 3.27 (br t, J=6.3 Hz, 2H), 2.68-2.65 (m, 3H), 2.59-2.47 (m, 2H), 2.27-2.19 (m, 2H), 2.12-2.04 (m, 2H), 2.03-1.94 (m, 2H), 1.94-1.84 (m, 2H)

### Example 152. N-(2-(cyclopentyl(2-fluoroethyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide

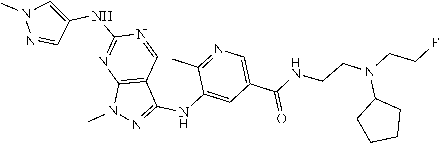 [SMILES: Cc1ncc(C(=O)NCCN(CCF)C2CCCC2)cc1Nc1nn(C)c2nc(Nc3cnn(C)c3)ncc12]

### Step a: tert-butyl (2-(cyclopentyl(2-fluoroethyl)amino)ethyl)carbamate

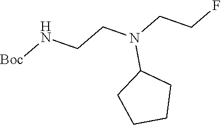 [SMILES: CC(C)(C)OC(=O)NCCN(CCF)C1CCCC1]

A mixture of N-(2-fluoroethyl)cyclopentanamine (0.25 g, 1.9 mmol) and K₂CO₃ (0.66 g, 4.8 mmol) in ACN (10 mL), then tert-butyl (2-bromoethyl)carbamate (0.47 g, 2.1 mmol) was added. The reaction mixture was stirred at 80° C. for 16 hours. The resulting mixture was quenched with water (10 mL) and extracted with ethyl acetate (20 mL×3). The combined organic phases were dried over anhydrous Na₂SO₄ and filtered. The filtrate was concentrated under reduced pressure to give crude product, which was purified by column:Xtimate C18 150*40 mm*5 um to give the title compound tert-butyl (2-(cyclopentyl(2-fluoroethyl)amino)ethyl)carbamate (200 mg, 38%) as a yellow oil. LCMS (ESI): mass calcd. for C₁₄H₂₇FN₂O₂, 274.375; m/z found, 275.2 [M+H]⁺.

### Step b: N¹-cyclopentyl-N¹-(2-fluoroethyl)ethane-1,2-diamine

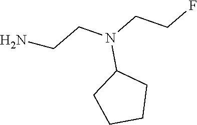 [SMILES: NCCN(CCF)C1CCCC1]

To a solution of tert-butyl (2-(cyclopentyl(2-fluoroethyl)amino)ethyl)carbamate (150 mg, 0.55 mmol) in DCM (10 mL) was added HCl/dioxane (1 mL, 4 mmol, 4M). The resultant mixture was stirred at room temperature for 1 h before quenched with water (0.5 mL) at 0° C. The resulting mixture was concentrated to dryness under reduced pressure to afford the crude product N¹-cyclopentyl-N¹-(2-fluoroethyl)ethane-1,2-diamine (120 mg, 89%) as a yellow oil.

### Step c: N-(2-(cyclopentyl(2-fluoroethyl)amino)ethyl)-6-methyl-5-((1-methyl-6-((1-methyl-1H-pyrazol-4-yl amino)-1H-pyrazolo[3,4-d]pyrimidin-3-yl)amino)nicotinamide

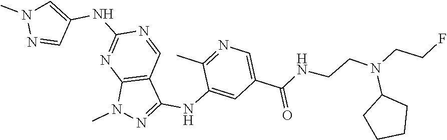 [SMILES: Cc1ncc(C(=O)NCCN(CCF)C2CCCC2)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (110 mg, 0.26 mmol), HATU (120 mg, 0.31 mmol) and N,N-diisopropylethylamine (102 mg, 0.79 mmol) in DMF (5 mL) was added Ni-cyclopentyl-N¹-(2-fluoroethyl)ethane-1,2-diamine. The mixture stirred at 25° C. for 2 hours and then concentrated under vacuum to give the crude product, which was purified by preparative high-performance liquid chromatography over column: Boston Uni C18 40*150*5 um to give the title compound N-(2-(cyclopentyl(2-fluoroethyl)amino)ethyl)-6-methyl-5-((1-methyl-6-((1-methyl-1H-pyrazol-4-yl)amino)-1H-pyrazolo[3,4-d]pyrimidin-3-yl)amino)nicotinamide (49.1 mg, 32%) as a yellow solid. LCMS (ESI): mass calcd. for C₂₆H₃₄FN₁₁O, 535.6; m/z found, 536.4 [M+H]⁺. ¹H NMR (400 MHz, METHANOL-d₄) δ 8.86 (s, 1H), 8.73 (d, J=2.0 Hz, 1H), 8.44 (d, J=1.5 Hz, 1H), 8.47-8.41 (m, 1H), 8.09 (s, 1H), 7.65 (s, 1H), 4.60-4.57 (m, 1H), 4.47 (t, J=5.3 Hz, 1H), 3.91 (s, 3H), 3.86 (s, 3H), 3.49 (t, J=6.9 Hz, 2H), 3.24-3.13 (m, 1H), 2.95 (t, J=5.3 Hz, 1H), 2.89 (t, J=5.1 Hz, 1H), 2.82 (t, J=7.0 Hz, 2H), 2.65 (s, 3H), 1.87 (br d, J=6.8 Hz, 2H), 1.67 (br d, J=3.8 Hz, 2H), 1.56 (br dd, J=4.9, 7.7 Hz, 2H), 1.47-1.33 (m, 2H).

### Example 153. N-(2-(7-azabicyclo[2.2.1]heptan-7-yl)ethyl)-6-methyl-5-((1-methyl-6-((1-methyl-1H-pyrazol-4-yl)amino)-1H-pyrazolo[3,4-d]pyrimidin-3-yl)amino)nicotinamide

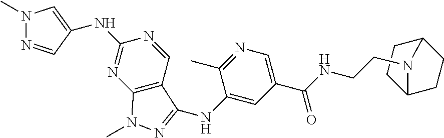 [SMILES: Cc1ncc(C(=O)NCCN2C3CCC2CC3)cc1Nc1nn(C)c2nc(Nc3cnn(C)c3)ncc12]

To a solution of 6-methyl-5-((1-methyl-6-((1-methyl-1H-pyrazol-4-yl)amino)-1H-pyrazolo[3,4-d]pyrimidin-3-yl)amino)nicotinic acid (200 mg, 0.49 mmol) in DMF (3 mL) was added 2-(7-azabicyclo[2.2.1]heptan-7-yl)ethanamine (100 mg, 0.74 mmol), DIEA (0.85 mL, 5.1 mmol) and HATU (580 mg, 1.5 mmol). The mixture stirred at room temperature for 2 h and then concentrated under vacuum to give the crude product, which was purified by column chromatography over silica gel (eluent: DCM/MeOH from 100:0 to 90:10) to give the crude product. Which was purified by preparative high-performance liquid chromatography over column: Welch Xtimate C18 150*30 mm*5 um to give the title compound N-(2-(7-azabicyclo[2.2.1]heptan-7-yl)ethyl)-6-methyl-5-((1-methyl-6-((1-methyl-1H-pyrazol-4-yl)amino)-1H-pyrazolo[3,4-d]pyrimidin-3-yl)amino)nicotinamide (40.1 mg, 16%) as a brown solid. LCMS (ESI): mass calcd. for C₂₅H₃₁N₁₁O, 501.6 m/z found, 502.3 [M+H]⁺. ¹H NMR (400 MHz, METHANOL-d₄) δ 8.87 (s, 1H), 8.76 (d, J=1.91 Hz, 1H), 8.49 (d, J=1.91 Hz, 1H), 8.10 (s, 1H), 7.68 (s, 1H), 3.93 (s, 3H), 3.87 (s, 3H), 3.58 (t, J=6.91 Hz, 2H), 3.51 (br s, 2H), 2.76 (t, J=6.85 Hz, 2H), 2.67 (s, 3H), 1.86 (br d, J=7.39 Hz, 4H), 1.45 (br d, J=7.51 Hz, 4H).

### Example 154. N-(2-(5-azaspiro[2.4]heptan-5-yl)ethyl)-5-((6-((1,3-dimethyl-1H-pyrazol-4-yl)amino)-1-methyl-1H-pyrazolo[3,4-d]pyrimidin-3-yl)amino)-6-methylnicotinamide

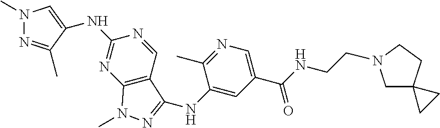 [SMILES: Cc1nn(C)cc1Nc1ncc2c(Nc3cc(C(=O)NCCN4CCC5(CC5)C4)cnc3C)nn(C)c2n1]

To a solution of 5-((6-((1,3-dimethyl-1H-pyrazol-4-yl)amino)-1-methyl-1H-pyrazolo[3,4-d]pyrimidin-3-yl)amino)-6-methylnicotinic acid (150 mg, 0.29 mmol) in DMF (4 mL) was added 2-(5-azaspiro[2.4]heptan-5-yl)ethanamine (68 mg, 0.49 mmol), DIEA (0.25 mL, 1.5 mmol) and HATU (150 mg, 0.39 mmol). The reaction mixture was stirred for 2 hours at room temperature. The mixture was concentrated under vacuum to afford a yellow solid which was purified by preparative high-performance liquid chromatography over column: Phenomenex C18 75*30 mm*3 um to give the title compound N-(2-(5-azaspiro[2.4]heptan-5-yl)ethyl)-5-((6-((1,3-dimethyl-1H-pyrazol-4-yl)amino)-1-methyl-1H-pyrazolo[3,4-d]pyrimidin-3-yl)amino)-6-methylnicotinamide (61 mg, 39%) as a white solid. LCMS (ESI): mass calcd. for C₂₆H₃₃N₁₁O, 515.3; m/z found, 516.3 [M+H]⁺. 1H NMR (400 MHz, METHANOL-d₄) δ 8.87 (s, 1H), 8.77 (d, J=2.01 Hz, 1H), 8.50 (d, J=1.51 Hz, 1H), 7.98 (br s, 1H), 3.87 (s, 3H), 3.82 (s, 3H), 3.57 (t, J=6.65 Hz, 2H), 2.83-2.90 (m, 2H), 2.77 (br t, J=6.65 Hz, 2H), 2.67 (s, 3H), 2.64 (s, 2H), 2.25 (s, 3H), 1.88 (t, J=7.03 Hz, 2H), 0.63 (br s, 2H), 0.59 (br s, 2H).

### Example 155. (S)-5-((6-((1,5-dimethyl-1H-pyrazol-4-yl)amino)-1-methyl-1H-pyrazolo[3,4-d]pyrimidin-3-yl)amino)-6-methyl-N-(2-(2-methylpyrrolidin-1-yl)ethyl)nicotinamide

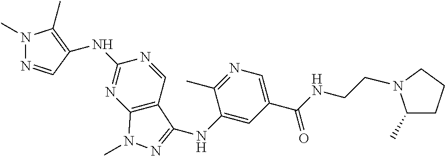 [SMILES: Cc1ncc(C(=O)NCCN2CCC[C@@H]2C)cc1Nc1nn(C)c2nc(Nc3cnn(C)c3C)ncc12]

To the mixture of 5-(6-(1,5-dimethyl-1H-pyrazol-4-yl)amino)-1-methyl-1H-pyrazolo[3,4-d]pyrimidin-3-yl)amino)-6-methylnicotinic acid (200 mg, 0.45 mmol) in DMF (5 mL) was added (S)-2-(2-methylpyrrolidin-1-yl)ethanamine (60 mg, 0.5 mmol), DIEA (0.3 mL, 1.8 mmol) and HATU (200 mg, 0.5 mmol). The resulting mixture was heated at room temperature and stirred for 2 hours. and then concentrated under vacuum to give the crude product, which was purified by preparative high-performance liquid chromatography over column: Welch Xtimate C18 150*30 mm*5 um to give the title compound (S)-5-((6-((1,5-dimethyl-1H-pyrazol-4-yl)amino)-1-methyl-1H-pyrazolo[3,4-d]pyrimidin-3-yl)amino)-6-methyl-N-(2-(2-methylpyrrolidin-1-yl)ethyl)nicotinamide (43.7 mg, 19%) as a white solid. LCMS (ESI): mass calcd. for C₂₅H₃₃N₁₁O, 503.6; m/z found, 504.3 [M+H]⁺. ¹H NMR (400 MHz, METHANOL-d₄) δ 8.84 (s, 1H), 8.75 (d, J=1.91 Hz, 1H), 8.48 (d, J=2.03 Hz, 1H), 7.66 (br s, 1H), 3.83 (s, 3H), 3.77 (s, 3H), 3.58-3.70 (m, 1H), 3.44-3.56 (m, 1H), 3.21-3.21-3.31 (m, 1H), 3.04-3.15 (m, 1H), 2.66 (s, 3H), 2.30-2.51 (m, 3H), 2.27 (s, 3H), 1.95-2.07 (m, 1H), 1.73-1.86 (m, 2H), 1.36-1.51 (m, 1H), 1.15 (d, J=6.08 Hz, 3H)

### Example 156. 5-((6-((1,5-dimethyl-1H-pyrazol-4-yl)amino)-1-methyl-1H-pyrazolo[3,4-d]pyrimidin-3-yl)amino)-N-(2-(2,2-dimethylpyrrolidin-1-yl)ethyl)-6-methylnicotinamide

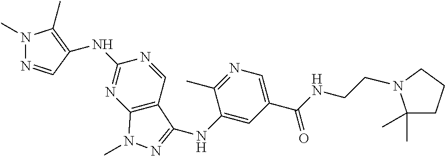 [SMILES: Cc1ncc(C(=O)NCCN2CCCC2(C)C)cc1Nc1nn(C)c2nc(Nc3cnn(C)c3C)ncc12]

To the mixture of 5-(6-(1,5-dimethyl-1H-pyrazol-4-yl)amino)-1-methyl-1H-pyrazolo[3,4-d]pyrimidin-3-yl)amino)-6-methylnicotinic acid (200 mg, 0.45 mmol) in DMF (5 mL) was added 2-(2,2-dimethylpyrrolidin-1-yl)ethanamine (70 mg, 0.49 mmol), DIEA (0.3 mL, 1.8 mmol) and HATU (200 mg, 0.52 mmol). The resulting mixture was heated at room temperature and stirred for 2 hours. And then concentrated under vacuum to give the crude product, which was purified by preparative high-performance liquid chromatography over column: Welch Xtimate C18 150*30 mm*5 um to give the title compound 5-((6-((1,5-dimethyl-1H-pyrazol-4-yl)amino)-1-methyl-1H-pyrazolo[3,4-d]pyrimidin-3-yl)amino)-N-(2-(2,2-dimethylpyrrolidin-1-yl)ethyl)-6-methylnicotinamide (66.9 mg, 27%) as a yellow solid LCMS (ESI): mass calcd. for C₂₆H₃₅N₁₁O, 517.6; m/z found, 518.3 [M+H]⁺. ¹H NMR (400 MHz, CHLOROFORM-d) δ ppm 8.54 (s, 1H), 8.47 (dd, J=12.46, 1.85 Hz, 2H), 7.79 (br s, 1H), 7.04 (br d, J=12.16 Hz, 1H), 6.60 (brs, 1H), 6.14 (s, 1H), 3.84 (d, J=5.01 Hz, 6H), 3.49 (q, J=5.17 Hz, 2H), 2.78 (br t, J=6.74 Hz, 2H), 2.66 (s, 3H), 2.61-2.66 (m, 2H), 2.26 (s, 3H), 1.73-1.83 (m, 2H), 1.68 (br s, 2H), 0.85-1.09 (m, 6H)

### Example 157. N-(2-(2,2-dimethylpyrrolidin-1-yl)ethyl)-6-methyl-5-((1-methyl-6-((1-methyl-1H-pyrazol-5-yl)amino)-1H-pyrazolo[3,4-d]pyrimidin-3-yl)amino)nicotinamide

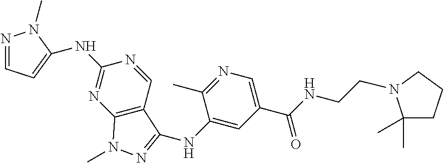 [SMILES: Cc1ncc(C(=O)NCCN2CCCC2(C)C)cc1Nc1nn(C)c2nc(Nc3ccnn3C)ncc12]

To a solution of 6-methyl-5-((1-methyl-6-((1-methyl-1H-pyrazol-5-yl)amino)-1H-pyrazolo[3,4-d]pyrimidin-3-yl)amino)nicotinic acid (200 mg, 0.30 mmol), 2-(2,2-dimethylpyrrolidin-1-yl)ethanamine (49.2 mg, 0.35 mmol), N-ethyl-N-isopropylpropan-2-amine (118 mg, 0.91 mmol) in DMF (5 mL) was added HATU (174 mg, 0.46 mmol) at room temperature. The reaction was stirred at room temperature for 2 h. The reaction mixture was concentrated in vacuo to give the crude product, which was purified by preparative high-performance liquid chromatography over column: Boston Green ODS 150*30 mm*5 um to give the title compound N-(2-(2,2-dimethylpyrrolidin-1-yl)ethyl)-6-methyl-5-((1-methyl-6-((1-methyl-1H-pyrazol-5-yl)amino)-1H-pyrazolo[3,4-d]pyrimidin-3-yl)amino)nicotinamide (25.5 mg, 17%) as a white solid. LCMS (ESI): mass calcd. for C₂₅H₃₃N₁₁O, 503.6; m/z found, 504.4 [M+H]⁺. ¹H NMR (400 MHz, METHANOL-d₄) δ 8.97 (s, 1H), 8.84 (d, J=1.8 Hz, 1H), 8.52 (d, J=1.8 Hz, 1H), 7.48 (d, J=2.0 Hz, 1H), 6.42 (d, J=1.9 Hz, 1H), 3.80 (d, J=2.7 Hz, 6H), 3.76 (br t, J=6.2 Hz, 2H), 3.58 (br s, 1H), 3.28 (br s, 3H), 2.69 (s, 3H), 2.18-2.07 (m, 2H), 2.06-2.00 (m, 2H), 1.39 (s, 6H)

### Example 158. (S)-5-((6-((1,3-dimethyl-1H-pyrazol-5-yl)amino)-1-methyl-1H-pyrazolo[3,4-d]pyrimidin-3-yl)amino)-6-methyl-N-(2-(2-methylpyrrolidin-1-yl)ethyl)nicotinamide

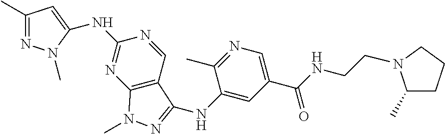 [SMILES: Cc1cc(Nc2ncc3c(Nc4cc(C(=O)NCCN5CCC[C@@H]5C)cnc4C)nn(C)c3n2)n(C)n1]

### Step a: Methyl 5-((6-((6,7-dihydro-5H-pyrazolo[5,1-b][1,3]oxazin-3-yl)amino)-1-methyl-1H-pyrazolo[3,4-d]pyrimidin-3-yl)amino)-6-methylnicotinate

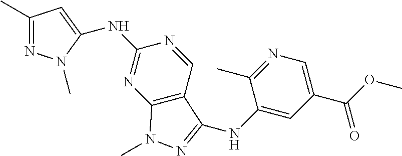

To a solution of methyl 5-((6-chloro-1-methyl-1H-pyrazolo[3,4-d]pyrimidin-3-yl)amino)-6-methylnicotinate (400 mg, 1.2 mmol), 1,3-dimethyl-1H-pyrazol-5-amine (152 mg, 1.37 mmol), Brettphos-Pd-G₃ (272 mg, 0.3 mmol) and Brettphos (161 mg, 0.30 mmol) in DMF (10 mL) was added Cs₂CO₃ (1.2 g, 3.6 mmol) at N₂ atmosphere. The reaction mixture was stirred at 90° C. overnight. The reaction mixture was concentrated under reduced pressure to give the crude product methyl 5-((6-((6,7-dihydro-5H-pyrazolo[5,1-b] [1,3]oxazin-3-yl)amino)-1-methyl-1H-pyrazolo[3,4-d]pyrimidin-3-yl)amino)-6-methylnicotinate (1.0 g, 49%) as yellow solid. LCMS (ESI): mass calcd. for C₁₉H₂₁N₉O₂, 407.4; m/z found, 408.2 [M+H]⁺.

### Step b: 5-((6-((6,7-dihydro-5H-pyrazolo[5,1-b][1,3]oxazin-3-yl)amino)-1-methyl-1H-pyrazolo[3,4-d]pyrimidin-3-yl)amino)-6-methylnicotinic acid

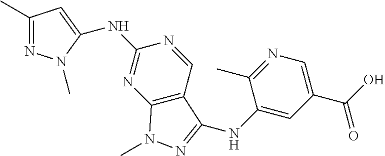 [SMILES: Cc1cc(Nc2ncc3c(Nc4cc(C(=O)O)cnc4C)nn(C)c3n2)n(C)n1]

To a solution of Methyl 5-((6-((1,3-dimethyl-1H-pyrazol-5-yl)amino)-1-methyl-1H-pyrazolo[3,4-d]pyrimidin-3-yl)amino)-6-methylnicotinate (900 mg, 0.534 mmol) in EtOH (10 mL) was added sodium hydroxide (2 mL, 4 mmol, 4M). The mixture was stirred at room temperature for 1 h. The mixture was adjusted to pH=3-4 with HCl (aq, 2 M). The mixture was filtered and washed with H₂O (20 mL×3). The solid was evaporated under vacuum to give desired product 5-((6-((6,7-dihydro-5H-pyrazolo[5,1-b][1,3]oxazin-3-yl)amino)-1-methyl-1H-pyrazolo[3,4-d]pyrimidin-3-yl)amino)-6-methylnicotinic acid (200 mg, 59%) as black solid. LCMS (ESI): mass calcd. for C₈H₁₉N₉O₂, 393.4; m/z found, 394.2 [M+H]⁺.

### Step c: (S)-5-((6-((1,3-dimethyl-1H-pyrazol-5-yl)amino)-1-methyl-1H-pyrazolo[3,4-d]pyrimidin-3-yl)amino)-6-methyl-N-(2-(2-methylpyrrolidin-1-yl)ethyl)nicotinamide

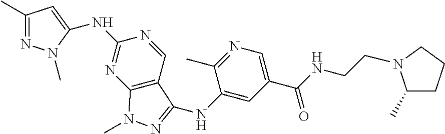 [SMILES: Cc1cc(Nc2ncc3c(Nc4cc(C(=O)NCCN5CCC[C@@H]5C)cnc4C)nn(C)c3n2)n(C)n1]

To a solution of 5-((6-((1,3-dimethyl-1H-pyrazol-5-yl)amino)-1-methyl-1H-pyrazolo[3,4-d]pyrimidin-3-yl)amino)-6-methylnicotinic acid (180 mg, 0.29 mmol), (S)-2-(2-methylpyrrolidin-1-yl)ethanamine (41.6 mg, 0.33 mmol), DIEA (110 mg, 0.86 mmol) in DMF (5 mL) was added HATU (163 mg, 0.43 mmol) at room temperature. The reaction was stirred at room temperature for 2 h. The reaction mixture was concentrated in vacuo to give the crude product, which was purified by preparative high-performance liquid chromatography over column: Welch Xtimate C18 150*30 mm*5 um to give the title compound (S)-5-((6-((1,3-dimethyl-1H-pyrazol-5-yl)amino)-1-methyl-1H-pyrazolo[3,4-d]pyrimidin-3-yl)amino)-6-methyl-N-(2-(2-methylpyrrolidin-1-yl)ethyl)nicotinamide (30.4 mg, 21%) as a white solid. LCMS (ESI): mass calcd. for C₂₅H₃₃N₁₁O, 503.6; m/z found, 504.3 [M+H]⁺. ¹H NMR (400 MHz, METHANOL-d₄) δ 8.94 (s, 1H), 8.79 (d, J=1.8 Hz, 1H), 8.49 (d, J=1.8 Hz, 1H), 6.22 (s, 1H), 3.80 (s, 3H), 3.71 (s, 3H), 3.65 (ddd, J=5.1, 8.2, 13.4 Hz, 1H), 3.55-3.45 (m, 1H), 3.30-3.24 (m, 1H), 3.10 (td, J=7.6, 12.2 Hz, 1H), 2.67 (s, 3H), 2.52-2.43 (m, 1H), 2.41-2.28 (m, 2H), 2.25 (s, 3H), 2.07-1.96 (m, 1H), 1.86-1.76 (m, 2H), 1.51-1.40 (m, 1H), 1.16 (d, J=6.1 Hz, 3H)

### Example 159. 5-((6-((1,3-dimethyl-1H-pyrazol-4-yl)amino)-1-methyl-1H-pyrazolo[3,4-d]pyrimidin-3-yl)amino)-N-(2-(3,3-dimethylpyrrolidin-1-yl)ethyl)-6-methylnicotinamide

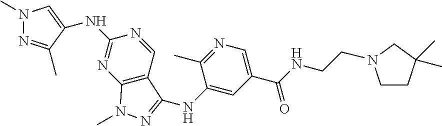

To a solution of 5-((6-((1,3-dimethyl-1H-pyrazol-4-yl)amino)-1-methyl-1H-pyrazolo[3,4-d]pyrimidin-3-yl)amino)-6-methylnicotinic acid (100 mg, 0.19 mmol) in DMF (3 mL) was added 2-(3,3-dimethylpyrrolidin-1-yl)ethanamine (45 mg, 0.32 mmol), DIEA (0.2 mL, 1.2 mmol) and HATU (135 mg, 0.36 mmol). The mixture stirred at room temperature for 1 h and then concentrated under vacuum to give the crude product, which was purified by preparative high-performance liquid chromatography over column: Phenomenex C18 75*30 mm*3 um to give the title compound 5-((6-((1,3-dimethyl-1H-pyrazol-4-yl)amino)-1-methyl-1H-pyrazolo[3,4-d]pyrimidin-3-yl)amino)-N-(2-(3,3-dimethylpyrrolidin-1-yl)ethyl)-6-methylnicotinamide (47.4 mg, 44%) as a white solid. LCMS (ESI): mass calcd. for C₂₆H₃₅N₁₁O, 517.6, m/z found, 518.3 [M+H]⁺. ¹H NMR (400 MHz, DMSO-d₆) δ ppm 9.03-9.19 (m, 1H), 8.94 (s, 1H), 8.64 (s, 1H), 8.46-8.49 (m, 1H), 8.46 (s, 1H), 8.39 (s, 1H), 7.90-8.13 (m, 1H), 3.78 (s, 3H), 3.74-3.76 (m, 3H), 3.41 (br s, 2H), 2.58-2.61 (m, 5H), 2.56 (br d, J=6.80 Hz, 3H), 2.29-2.32 (m, 2H), 2.16 (s, 2H), 1.50 (t, J=7.03 Hz, 2H), 1.03 (s, 6H).

### Example 160. (S)-5-((6-((1,3-dimethyl-1H-pyrazol-4-yl)amino)-1-methyl-1H-pyrazolo[3,4-d]pyrimidin-3-yl)amino)-6-methyl-N-(2-(2-methylpyrrolidin-1-yl)ethyl)nicotinamide

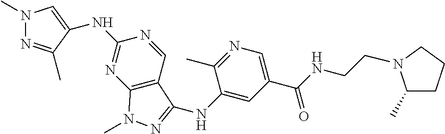 [SMILES: Cc1nn(C)cc1Nc1ncc2c(Nc3cc(C(=O)NCCN4CCC[C@@H]4C)cnc3C)nn(C)c2n1]

To a solution of 5-((6-((1,3-dimethyl-1H-pyrazol-4-yl)amino)-1-methyl-1H-pyrazolo[3,4-d]pyrimidin-3-yl)amino)-6-methylnicotinic acid (140 mg, 0.27 mmol) in DMF (3 mL) was added (S)-2-(2-methylpyrrolidin-1-yl)ethanamine (50 mg, 0.39 mmol), DIEA (0.25 mL, 1.5 mmol) and HATU (177 mg, 0.46 mmol). The mixture stirred at room temperature for 15 h and then concentrated under vacuum to give the crude product, which was purified by preparative high-performance liquid chromatography over column: Phenomenex C18 75*30 mm*3 um to give the title compound (S)-5-((6-((1,3-dimethyl-1H-pyrazol-4-yl)amino)-1-methyl-1H-pyrazolo[3,4-d]pyrimidin-3-yl)amino)-6-methyl-N-(2-(2-methylpyrrolidin-1-yl)ethyl)nicotinamide (55.5 mg, 38%) as a white solid. LCMS (ESI): mass calcd. for C₂₅H₃₃N₁₁O, 503.6 m/z; found, 504.3 [M+H]⁺. ¹H NMR (400 MHz, METHANOL-d₄) δ 8.87 (s, 1H), 8.76 (d, J=2.03 Hz, 1H), 8.48 (d, J=2.03 Hz, 1H), 7.98 (br s, 1H), 3.86 (s, 3H), 3.82 (s, 3H), 3.62-3.69 (m, 1H), 3.47-3.53 (m, 1H), 3.24-3.30 (m, 1H), 3.07-3.13 (m, 1H), 2.66 (s, 3H), 2.43-2.49 (m, 1H), 2.27-2.37 (m, 2H), 2.24 (s, 3H), 1.98-2.04 (m, 1H), 1.77-1.84 (m, 2H), 1.40-1.48 (m, 1H), 1.15 (d, J=6.08 Hz, 3H).

### Example 161. 5-((6-((1,3-dimethyl-1H-pyrazol-4-yl)amino)-1-methyl-1H-pyrazolo[3,4-d]pyrimidin-3-yl)amino)-N-(2-(2,2-dimethylpyrrolidin-1-yl)ethyl)-6-methylnicotinamide

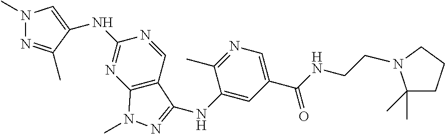 [SMILES: Cc1nn(C)cc1Nc1ncc2c(Nc3cc(C(=O)NCCN4CCCC4(C)C)cnc3C)nn(C)c2n1]

### Step a: methyl 5-((6-((1,3-dimethyl-1H-pyrazol-4-yl)amino)-1-methyl-1H-pyrazolo[3,4-d]pyrimidin-3-yl)amino)-6-methylnicotinate

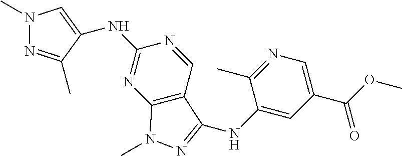

To a solution of methyl 5-((6-chloro-1-methyl-1H-pyrazolo[3,4-d]pyrimidin-3-yl)amino)-6-methylnicotinate (2 g, 6.0 mmol) in i-PrOH (20 mL) was added DIEA (2.7 mL, 15.5 mmol) and 1,3-dimethyl-1H-pyrazol-4-amine (1 g, 9.0 mmol). The mixture was stirred at 110° C. for 48 hours. The reaction mixture was concentrated to dryness in vacuo to give a green solid. The green solid was subjected to column chromatography over silica gel (gradient elution: 0-20% methanol in dichloromethane). The pure fractions were collected and concentrated to dryness in vacuum to give the product methyl 5-((6-((1,3-dimethyl-1H-pyrazol-4-yl)amino)-1-methyl-1H-pyrazolo[3,4-d]pyrimidin-3-yl)amino)-6-methylnicotinate (2.1 g, 83%) as a green solid. LCMS (ESI): mass calcd. for C₁₉H₂₁N₉O₂, 407.4; m/z found, 408.2 [M+H]⁺.

### Step b: 5-((6-((1,3-dimethyl-1H-pyrazol-4-yl)amino)-1-methyl-1H-pyrazolo[3,4-d]pyrimidin-3-yl)amino)-6-methylnicotinic acid

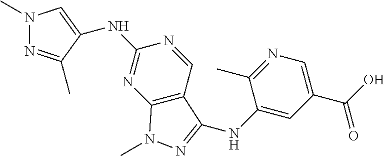 [SMILES: Cc1nn(C)cc1Nc1ncc2c(Nc3cc(C(=O)O)cnc3C)nn(C)c2n1]

To a solution of methyl 5-((6-((1,3-dimethyl-1H-pyrazol-4-yl)amino)-1-methyl-1H-pyrazolo[3,4-d]pyrimidin-3-yl)amino)-6-methylnicotinate (2.1 g, 5.0 mmol) in THF (27 mL) and H₂O (9 mL) was added LiOH— H₂O (670 mg, 16 mmol) at room temperature over 1 min. The mixture was stirred at room temperature for 1 hour. The reaction mixture was acidified with 1N HCl to pH=5. The mixture was filtered, rinsed with H₂O (5 mL). The filter cake was dried in vacuo to afford 5-((6-((1,3-dimethyl-1H-pyrazol-4-yl)amino)-1-methyl-1H-pyrazolo[3,4-d]pyrimidin-3-yl)amino)-6-methylnicotinic acid (1.41 g, 54%) as a green solid. LCMS (ESI): mass calcd. for C₁₈H₁₉N₉O₂, 393.4; m/z found, 394.1 [M+H]⁺.

### Step c: 5-((6-((1,3-dimethyl-1H-pyrazol-4-yl)amino)-1-methyl-1H-pyrazolo[3,4-d]pyrimidin-3-yl)amino)-N-(2-(2,2-dimethylpyrrolidin-1-yl)ethyl)-6-methylnicotinamide

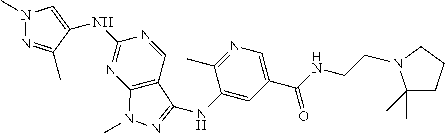 [SMILES: Cc1nn(C)cc1Nc1ncc2c(Nc3cc(C(=O)NCCN4CCCC4(C)C)cnc3C)nn(C)c2n1]

To the mixture of 5-((6-((1,3-dimethyl-1H-pyrazol-4-yl)amino)-1-methyl-1H-pyrazolo[3,4-d]pyrimidin-3-yl)amino)-6-methylnicotinic acid (150 mg, 0.29 mmol) in DMF (3 mL) was added 2-(2,2-dimethylpyrrolidin-1-yl)ethanamine (49 mg, 0.34 mmol) and DIEA (0.2 mL, 1.21 mmol), then HATU (150 mg, 0.39 mmol) was added. The resulting mixture was stirred at room temperature for 1 hour. The reaction mixture was concentrated to dryness in vacuo to afford a black oil, which was purified by preparative by preparative high-performance liquid chromatography over column: Boston Prime C18 150*30 mm*5 um to give the title compound 5-((6-((1,3-dimethyl-1H-pyrazol-4-yl)amino)-1-methyl-1H-pyrazolo[3,4-d]pyrimidin-3-yl)amino)-N-(2-(2,2-dimethylpyrrolidin-1-yl)ethyl)-6-methylnicotinamide (32.4 mg, 21%) as a yellow powder. LCMS (ESI): mass calcd. for C₂₆H₃₅N₁₁O, 517.6; m/z found: 518.3 [M+H]⁺. ¹H NMR (400 MHz, METHANOL-d₄) δ 8.87 (s, 1H), 8.76 (d, J=1.91 Hz, 1H), 8.47 (d, J=2.03 Hz, 1H), 7.98 (br s, 1H), 3.87 (s, 3H), 3.82 (s, 3H), 3.50-3.55 (m, 2H), 2.93 (br t, J=7.27 Hz, 2H), 2.66-2.71 (m, 5H), 2.25 (s, 3H), 1.84 (br d, J=7.27 Hz, 2H), 1.71 (br d, J=8.34 Hz, 2H), 1.06 (s, 6H).

### Example 162. (S)-5-((6-((1,5-dimethyl-1H-pyrazol-4-yl)amino)-1-methyl-1H-pyrazolo[3,4-d]pyrimidin-3-yl)amino)-6-methyl-N-(2-(2-methylpyrrolidin-1-yl)ethyl)nicotinamide

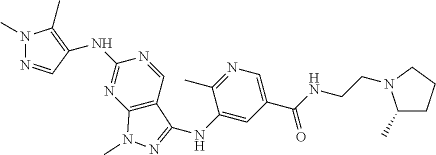 [SMILES: Cc1ncc(C(=O)NCCN2CCC[C@@H]2C)cc1Nc1nn(C)c2nc(Nc3cnn(C)c3C)ncc12]

### Step a: methyl 5-((6-((1,5-dimethyl-1H-pyrazol-4-yl)amino)-1-methyl-1H-pyrazolo[3,4-d]pyrimidin-3-yl)amino)-6-methylnicotinate

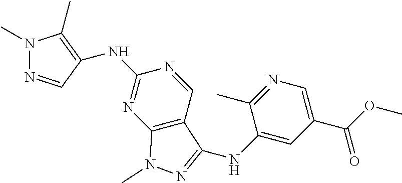

To a solution of methyl 5-((6-chloro-1-methyl-1H-pyrazolo[3,4-d]pyrimidin-3-yl)amino)-6-methylnicotinate (2 g, 6.0 mmol) in i-PrOH (10 mL) was added N-ethyl-N-isopropylpropan-2-amine (2.5 mL, 15.1 mmol) and 1,5-dimethyl-1H-pyrazol-4-amine (1.3 g, 11.7 mmol). The mixture was stirred at 110° C. for 48 hours. The reaction mixture was concentrated to dryness in vacuo to give a green solid. The green solid was subjected to column chromatography over silica gel (gradient elution: 0-20% methanol in dichloromethane). The pure fractions were collected and concentrated to dryness in vacuum to give the product methyl 5-((6-((1,5-dimethyl-1H-pyrazol-4-yl)amino)-1-methyl-1H-pyrazolo[3,4-d]pyrimidin-3-yl)amino)-6-methylnicotinate (2.6 g, 87%) as a yellow solid.

LCMS (ESI): mass calcd. for C₁₉H₂₁N₉O₂, 407.4; m/z found, 408.2 [M+H]⁺.

### Step b: 5-((6-((1,5-dimethyl-1H-pyrazol-4-yl)amino)-1-methyl-1H-pyrazolo[3,4-d]pyrimidin-3-yl)amino)-6-methylnicotinic acid

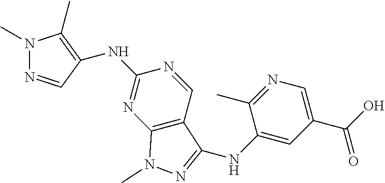 [SMILES: Cc1ncc(C(=O)O)cc1Nc1nn(C)c2nc(Nc3cnn(C)c3C)ncc12]

To a solution of methyl 5-((6-((1,5-dimethyl-1H-pyrazol-4-yl)amino)-1-methyl-1H-pyrazolo[3,4-d]pyrimidin-3-yl)amino)-6-methylnicotinate (2.6 g, 5.2 mmol) in THF/MeOH=1/1 (150 mL) was added lithium hydroxide (257 mg, 10.7 mmol) in H₂O (50 mL). The mixture was stirred at room temperature for 1 hour. The reaction mixture was acidified with 1N HCl to pH=5. The mixture was filtered, rinsed with H₂O (10 mL). The filter cake was dried in vacuo to afford 5-((6-((1,5-dimethyl-1H-pyrazol-4-yl)amino)-1-methyl-1H-pyrazolo[3,4-d]pyrimidin-3-yl)amino)-6-methylnicotinic acid (1.8 g, 79%) as a yellow solid. LCMS (ESI): mass calcd. for C₁₈H₁₉N₉O₂, 393.4; m/z found, 394.2 [M+H]⁺.

### Step c: (S)-5-((6-((1,5-dimethyl-1H-pyrazol-4-yl)amino)-1-methyl-1H-pyrazolo[3,4-d]pyrimidin-3-yl)amino)-6-methyl-N-(2-(2-methylpyrrolidin-1-yl)ethyl)nicotinamide

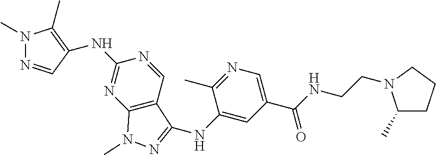 [SMILES: Cc1ncc(C(=O)NCCN2CCC[C@@H]2C)cc1Nc1nn(C)c2nc(Nc3cnn(C)c3C)ncc12]

To the mixture of 5-((6-((1,5-dimethyl-1H-pyrazol-4-yl)amino)-1-methyl-1H-pyrazolo[3,4-d]pyrimidin-3-yl)amino)-6-methylnicotinic acid (200 mg, 0.45 mmol) in DMF (5 mL) was added (S)-2-(2-methylpyrrolidin-1-yl)ethanamine (60 mg, 0.47 mmol) and DIEA (0.3 mL, 1.8 mmol), then HATU (200 mg, 0.53 mmol) was added. The resulting mixture was stirred at room temperature for 1 hour. The reaction mixture was concentrated to dryness in vacuo to afford a black oil, which was purified by preparative by preparative high-performance liquid chromatography over column: Welch Xtimate C18 150*30 mm*5 um to give the title compound (S)-5-((6-((1,5-dimethyl-1H-pyrazol-4-yl)amino)-1-methyl-1H-pyrazolo[3,4-d]pyrimidin-3-yl)amino)-6-methyl-N-(2-(2-methylpyrrolidin-1-yl)ethyl)nicotinamide (43.7 mg, 19%) as a white solid. LCMS (ESI): mass calcd. for C₂₅H₃₃N₁₁O, 503.6; m/z found: 504.3 [M+H]⁺. ¹H NMR (400 MHz, METHANOL-d₄) δ 8.84 (s, 1H), 8.75 (d, J=1.91 Hz, 1H), 8.48 (d, J=2.03 Hz, 1H), 7.66 (br s, 1H), 3.83 (s, 3H), 3.77 (s, 3H), 3.58-3.70 (m, 1H), 3.44-3.56 (m, 1H), 3.21-3.31 (m, 1H), 3.04-3.15 (m, 1H), 2.66 (s, 3H), 2.30-2.51 (m, 3H), 2.27 (s, 3H), 1.95-2.07 (m, 1H), 1.73-1.86 (m, 2H), 1.36-1.51 (m, 1H), 1.15 (d, J=6.08 Hz, 3H).

### Example 163.5-((6-((6,7-dihydro-5H-pyrazolo[5,1-b][1,3]oxazin-3-yl)amino)-1-methyl-1H-pyrazolo[3,4-d]pyrimidin-3-yl)amino)-N-(2-(2,2-dimethylpyrrolidin-1-yl)ethyl)-6-methylnicotinamide

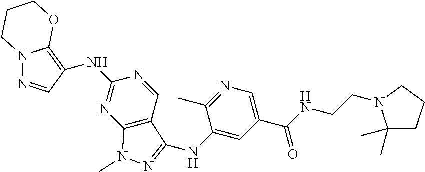 [SMILES: Cc1ncc(C(=O)NCCN2CCCC2(C)C)cc1Nc1nn(C)c2nc(Nc3cnn4c3OCCC4)ncc12]

### Step a: 3-nitro-6,7-dihydro-5H-pyrazolo[5,1-b][1,3]oxazine

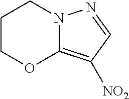 [SMILES: O=[N+]([O-])c1cnn2c1OCCC2]

To a solution of 6,7-dihydro-5H-pyrazolo[5,1-b][1,3]oxazine (1.5 g, 12.1 mmol) in sulfuric acid (10 mL) was added potassium nitrate (1.7 g, 17.1 mmol) at 0° C. The resultant mixture was stirred at 0° C. for 1 h. The mixture was adjusted to pH=7-8 with NaOH (aq, 2 M). The reaction mixture was concentrated to give the crude product, which was purified by column chromatography over silica gel (eluent: petroleum ether:ethyl acetate=0:100) to give the title compound to give the title compound 3-nitro-6,7-dihydro-5H-pyrazolo[5,1-b][1,3]oxazine (500 mg, 35%) as a white solid. ¹H NMR (400 MHz, METHANOL-d₄) δ 8.58 (s, 1H), 4.29 (t, J=7.2 Hz, 2H), 4.03 (t, J=7.0 Hz, 2H), 2.75 (quin, J=7.1 Hz, 2H)

### Step b: 6,7-dihydro-5H-pyrazolo[5,1-b][1,3]oxazin-3-amine

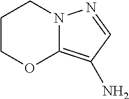

A solution of 3-nitro-6,7-dihydro-5H-pyrazolo[5,1-b][1,3]oxazine (400 mg, 2.4 mol) in ethanol (8 mL) was added Pd/C (248 mg, 0.23 mmol) under N₂. The suspension was degassed in vacuo and purged with H₂ several times. The mixture was stirred under H₂ balloon (15 psi) at room temperature for 1 h. After uptake of H₂, the reaction mixture was filtered. The filtrate was evaporated give the crude product 6,7-dihydro-5H-pyrazolo[5,1-b][1,3]oxazin-3-amine (248 mg, 0.23 mmol) as yellow oil. ¹H NMR (400 MHz, METHANOL-d₄) δ 7.14 (s, 1H), 3.84 (t, J=7.0 Hz, 2H), 3.63-3.58 (m, 2H), 2.67-2.60 (m, 2H)

### Step c: methyl 5-((6-((6,7-dihydro-5H-pyrazolo[5,1-b][1,3]oxazin-3-yl)amino)-1-methyl-1H-pyrazolo[3,4-d]pyrimidin-3-yl)amino)-6-methylnicotinate

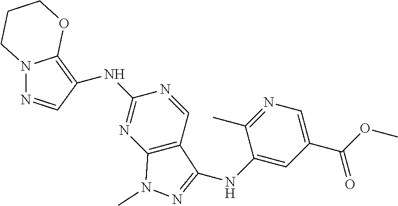 [SMILES: COC(=O)c1cnc(C)c(Nc2nn(C)c3nc(Nc4cnn5c4OCCC5)ncc23)c1]

To a solution of 5-((6-chloro-1-methyl-1H-pyrazolo[3,4-d]pyrimidin-3-yl)amino)-6-methylnicotinate (300 mg, 0.87 mmol), 6,7-dihydro-5H-pyrazolo[5,1-b][1,3]oxazin-3-amine (137 mg, 0.98 mmol), Brettphos-Pd-G3 (196 mg, 0.22 mmol), Brettphos (116 mg, 0.22 mmol) in DMF (7 mL) was added Cs₂CO₃ (0.85 g, 2.6 mmol) at N₂ atmosphere. The reaction mixture was stirred at 90° C. overnight. The reaction mixture was concentrated under reduced pressure to give the crude product, which was purified by a column on silica gel (eluent: petroleum ether:ethyl acetate=100/0 to petroleum ether:ethyl acetate=0/100). The pure fractions were collected and the solvent was evaporated to give the title product methyl 5-((6-((6,7-dihydro-5H-pyrazolo[5,1-b][1,3]oxazin-3-yl)amino)-1-methyl-1H-pyrazolo[3,4-d]pyrimidin-3-yl)amino)-6-methylnicotinate (100 mg, 27%) as yellow solid. LCMS (ESI): mass calcd. for C₂₀H₂₁N₉O₃, 435.4; m/z found: 435.7 [M+H]⁺.

### Step d: 5-((6-((6,7-dihydro-5H-pyrazolo[5,1-b][1,3]oxazin-3-yl)amino)-1-methyl-1H-pyrazolo[3,4-d]pyrimidin-3-yl)amino)-6-methylnicotinic acid

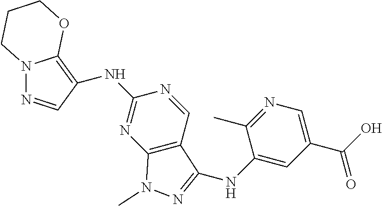 [SMILES: Cc1ncc(C(=O)O)cc1Nc1nn(C)c2nc(Nc3cnn4c3OCCC4)ncc12]

To a solution of Methyl 5-((6-((6,7-dihydro-5H-pyrazolo[5,1-b][1,3]oxazin-3-yl)amino)-1-methyl-1H-pyrazolo[3,4-d]pyrimidin-3-yl)amino)-6-methylnicotinate (95 mg, 0.22 mmol) in EtOH (5 mL) was added sodium hydroxide (2.2 mL, 4.5 mmol, 2M). The mixture was stirred at room temperature for 1 h. The mixture was adjust to pH=3-4 with HCl (aq, 2M). The mixture was filtered and washed with H₂O (20 mL×3). The solid was evaporated under vacuum to give desired product 5-((6-((6,7-dihydro-5H-pyrazolo[5,1-b][1,3]oxazin-3-yl)amino)-1-methyl-1H-pyrazolo[3,4-d]pyrimidin-3-yl)amino)-6-methylnicotinic acid (80 mg, 87%) as black solid. LCMS (ESI): mass calcd. for C₁₉H₁₉N₉O₃, 421.4; m/z found, 422.1 [M+H]⁺.

### Step e: 5-((6-((6,7-dihydro-5H-pyrazolo[5,1-b][1,3]oxazin-3-yl)amino)-1-methyl-1H-pyrazolo[3,4-d]pyrimidin-3-yl)amino)-N-(2-(2,2-dimethylpyrrolidin-1-yl)ethyl)-6-methylnicotinamide

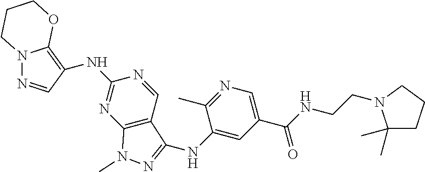 [SMILES: Cc1ncc(C(=O)NCCN2CCCC2(C)C)cc1Nc1nn(C)c2nc(Nc3cnn4c3OCCC4)ncc12]

To a solution of 5-((6-((6,7-dihydro-5H-pyrazolo[5,1-b][1,3]oxazin-3-yl)amino)-1-methyl-1H-pyrazolo[3,4-d]pyrimidin-3-yl)amino)-6-methylnicotinic acid (75 mg, 0.18 mmol), 2-(2,2-dimethylpyrrolidin-1-yl)ethanamine (28.7 mg, 0.20 mmol), DIEA (69.0 mg, 0.53 mmol) in DMF (5 mL) was added HATU (102 mg, 0.27 mmol) at room temperature. The reaction was stirred at room temperature for 2 h. The reaction mixture was concentrated in vacuo to give the crude product, which was purified by preparative high-performance liquid chromatography over column: Boston Green ODS 150*30 mm*5 um to give the title compound 5-((6-((6,7-dihydro-5H-pyrazolo[5,1-b][1,3]oxazin-3-yl)amino)-1-methyl-1H-pyrazolo[3,4-d]pyrimidin-3-yl)amino)-N-(2-(2,2-dimethylpyrrolidin-1-yl)ethyl)-6-methylnicotinamide (17 mg, 15%) as a yellow solid. LCMS (ESI): mass calcd. for C₂₇H₃₅N₁₁O₂, 545.3; m/z found: 546.3 [M+H]⁺. ¹H NMR (400 MHz, METHANOL-d₄) δ 8.88 (s, 1H), 8.79 (d, J=2.0 Hz, 1H), 8.47 (d, J=2.0 Hz, 1H), 8.25 (br s, 1H), 3.96 (t, J=7.0 Hz, 2H), 3.91 (t, J=6.8 Hz, 2H), 3.84 (s, 3H), 3.67 (br t, J=6.4 Hz, 2H), 3.37-3.33 (m, 2H), 3.07 (hr s, 2H), 2.75-2.69 (m, 2H), 2.66 (s, 3H), 2.07-1.97 (m, 2H), 1.95-1.87 (m, 2H), 1.27 (s, 6H).

### Biological Assays

### PDGFRβ HTRF Assay

### I. Materials

### Reagents

| Product | Vendor | Catalog# | Comments |
|---|---|---|---|
| 5× Kinase | Invitrogen by | PV6135 | Kinase Buffer A is supplied as a 5× |
| Buffer A | Thermo | | concentrated stock. For example, |
| | Fisher | | prepare a 1× solution by adding 4 mL |
| | Scientific | | of the 5× solution to 16 mL of distilled |
| | | | H2O. The 1× kinase reaction buffer is |
| | | | stable at room temperature. 1× Kinase |
| | | | Buffer A consists of 50 mM HEPES |
| | | | pH 7.5, 10 mM MgCl₂, 1 mM EGTA, |
| | | | 0.01% Brij-35. 100 mL per vial stored |
| | | | at room temperature |
| Molecular | Corning | 46-000-CM | 1 L vial stored at room temperature; |
| Biology Grade | | | LC-MS Grade |
| water | | | |
| DTT (DL | Sigma | D5545-1G | 1 G vial was received as a solid and |
| Dithiothreitol) | | | dissolved with 3241 uL water to |
| | | | prepare a 2M stock; aliquot DTT and |
| | | | store at −20° C. storage; discard aliquot |
| | | | at EOB; MW = 154.25 g/mol |
| Albumin from | Sigma | A2512 | Received 250 mg and dissolved to a |
| Chicken Egg | | | 10% solution in water (prepare fresh on |
| White | | | experimental day and discard at EOB); |
| | | | stored at 4 degrees; Lot#SLBN4912; |
| | | | MW = 44287 |
| His6-TEV- | Prepared at | | PDGFRb_08Prep02; 6.44 mg/mL or |
| PDGFRβ | Accelagen | | 116 μM Stock; |
| (557-1016) | | | MW = 55455.3 g/mol; Storage Buffer |
| | | | contains 50 mM Tris-HCI, pH 8.0, 200 |
| | | | mM NaCl, 10% glycerol, 1 mM DTT; |
| | | | protein aliquots stored at −80° C. storage |
| DrySolv | Millipore | MX1457-7 | Stored at RT |
| Methylsufoxide | | | |
| A 384-well | PerkinElmer | 6008289 | Stored at RT |
| assay plate, | | | |
| ProxiPlate | | | |
| Plus, shallow | | | |
| well, white | | | |
| SMART small | Thermo | 24073295 | |
| tube metal tip | Fisher | | |
| dispensing | Scientific | | |
| cassette | | | |
| HTRF | Cisbio | 62TK0PEC | 20,000 tests; 4° C. storage; Allow the |
| KinEASE-TK | | | contents of the Cisbio kit to warm up to |
| kit | | | room temperature before use |
| Adenosine 5′- | Sigma | A7699 | Prepare a 100 mM solution in water; |
| triphosphate | | | BioXtra, |
| (ATP) | | | ≥99% (HPLC); −20° C. storage |
| disodium salt | | | |
| hydrate | | | |

Instrumentation: a. Compound liquid handling: LabCyte Echo; b. Reagent liquid handling: Thermo Scientific Multidrop Combi; c. BMG PHERAStar multilabel plate reader.

Protein Reagent: His6-TEV-PDGFRβ Protein Prep prepared at Accelagen.

### II. Methods and Procedures

### Stock Solutions:

Assay buffer stock solution, contains 50 mM Hepes, 10 mM MgCl₂, 1 mM EGTA, and 0.01% Brij-35, 0.01% ovalbumin, 2 mM DTT at pH 7.5, in molecular biology grade water. Store at room temperature.

DTT, 2 M in molecular biology grade water, store at −20° C. in aliquots.

Ovalbumin, 10% or 100 mg/mL, prepare fresh on experimental day.

PDGFRβ, 116 μM (PDGFRb_08 Prep 02), produced at Accelagen. Store at −80° C. in aliquots.

TK-biotin peptide, 0.5 μM in molecular biology grade water, store at −20° C. in aliquots.

ATP, 100 mM in molecular biology grade water, store at −20° C. in aliquots.

HTRF KinEASE-TK kit: Allow the contents of the Cisbio kit to warm up to room temperature before use. This kit contains HTRF detection buffer, TK-Antibody labeled with Eu³⁺-cryptate, TK-substrate biotin and Streptavidin-XL665.

TK Substrate-Biotin, reconstitute 500 pg lyopholized with 574 μL molecular biology grade water to prepare a 500 μM stock; After use, aliquot the rest and store at −20° C.

TK Antibody-Cryptate, reconstitute lyophilized with 1 mL of molecular biology grade water (100× solution) then add 99 mL detection buffer to prepare a ready to use TK-antibody-cryptate solution; the concentration of the TK-antibody-cryptate reagent is not known. After use, aliquot the rest and store at −20° C.

Streptavidin-XL665, reconstitute 3 mg lyophilized with 3 mL molecular biology grade water to prepare a 1 mg/mL or 16.67 μM stock; MW=60 kDa; After use, aliquot the rest and store at −20° C.

### Freshly Prepared Solutions:

Assay buffer. Dilute 5× Kinase buffer 5-fold with molecular biology grade water and add DTT to 2 mM and ovalbumin to 0.1 mg/mL (or 0.01%).

2× protein solution. Make a working solution of 100 μM PDGFRβ in assay buffer. Keep on ice until use to maintain enzyme stability.

2× substrate solution. Make a working solution of 1.6 mM ATP and 1 μM TK-substrate biotin peptide in assay buffer. 3× quench/detection solution. Make a working solution of 0.1875 μM SA-XL665 and the TK-antibody cryptate diluted by H of total quench/detection volume in assay buffer.

Keep final streptavidin/biotin ratio at 1 to 8.

Example of 3× quench/detection solution preparation: 8 mL total volume.

1× assay buffer—3910 μL

TK antibody-cryptate in detection buffer—4000 μL

0.1875 μM SA-XL665—90 uL

The kinase reaction is stopped by the addition of the detection reagents which contain EDTA (detection step).

### Assay Procedure:

Assay in white ProxiPlate 384-well

Step 1. Dispensing inhibitors/DMSO and low control: Using the ECHO 555 acoustic dispenser, spot desired compound serial dilutions in DMSO, NEAT DMSO to represent the uninhibited enzyme control, and 10 μM final [imatinib] to the represent the 100% inhibited enzyme control

Step 2. PDGFRβ E+I pre-incubation: Add 2 μL 2× protein solution to columns 1-24 using the Multidrop Combi. Centrifuge at 1000 rpm for 1 min. Incubate 30 min at RT

Step 2. Enzymatic reaction: Add 2 μL substrate solution to columns 1-24 to initiate the reaction using the Multidrop Combi; cover/seal the assay plate to reduce evaporation. Centrifuge at 1000 rpm for 1 min. Incubate at room temperature for 3 hours.

Final concentrations of components in PDGFRp cascade assay:

50 mM Hepes, pH 7.5

10 mM MgCl2

0.01% Brij-35

1 mM EGTA

2 mM DTT

0.01% Ovalbumin

50 μM inactive PDGFRβ

0.5 μM TK-substrate biotin peptide

62.5 nM SA-XL-665

TK antibody-Eu3+-cryptate (diluted by 1/3 final from stock)

800 μM ATP

≤1% DMSO

Step 3. Quench/Detection: Add 2 μl 3× quench/detection solution to columns 1-24 using the Multidrop Combi; cover/seal the plate. Centrifuge 1 min 1000 rpm. Incubate at RT for 60 min. Read the plate in PHERAstar (or similar instrument) on HTRF setting at excitation 337 nm—dual emission—665/620 nm ratio.

### III. Calculations and Formulas

HTRF ratio values calculated by the instrument (Ratio is acceptor counts/donor counts*10,000) is exported from the plate reader and used in data analysis. The exported data will be used to calculate 1) compound activity and 2) assay statistics. Compound activity is represented by % Inhibition when testing a single dose of a compound or IC₅₀ when testing a dose response of a compound. Assay statistics can include Robust Z′ and Signal to Background.

% Inhibition Calculation: Percent inhibition will be calculated for sample wells based on the equation:

$$\% Inhibition = [ \frac{( x - cr}{( sr - cr )} ] * 100 )$$

Where, x: sample activity; cr: central reference is calculated based on wells containing all assay components and no compound (DMSO only); sr: scale reference is calculated based on wells inhibited with 10 μM Imatinib (these wells will contain the enzyme and substrate solutions)

IC₅₀ Calculation: For IC₅₀ determination, full 11-point dose response data will be processed using the following equation:

$$Y = {S}_{0} + \frac{{S}_{Inf} - {S}_{0}}{1 + {( \frac{IC 50}{1 {0}^{c}} )}^{n}}$$

Where S₀₁=Activity level at zero concentration of test compound; S_{Inf}-Activity level at infinite concentration; IC50: Concentration at which activity reaches 50% of maximum level; c=Concentration in logarithmic units corresponding to the values on the x-axis of the dose-response curve plot; Hill coefficient n=Measure of the slope at IC50. See Table 1, below.

Registered Parameters (when applicable): % Activity, IC₅₀, nHill Slope, S_{inf}, S₀, and Comments

Robust Z′ Calculation: Robust Z prime (RZ′) value will be calculated as defined by the following equation:

$${RZ}^{′} = 1 - \frac{{3}^{*} ( {RSD}_{cr} + {RSD}_{sr} )}{\vert {median}_{cr} - {median}_{sr} \vert}$$

Where, RSD: Robust standard deviation; cr: central reference is calculated based on wells containing all assay components and no compound (DMSO only); sr: scale reference is calculated based on wells inhibited with 10 μM Imatinib compound (these wells will contain the enzyme and substrate solutions)

### Signal to Background, S/B, Calculation

$$\frac{S}{B} = \frac{median ( CR )}{median ( SR )}$$

Where, CR, Central Reference (no compound wells); SR, Scale Reference (inhibitor control wells).

### PDGFRβ LanthaScreen Assay

### I. Materials

| Reagent | Manufacturer | Catalog # | Lot # |
|---|---|---|---|
| MilliQ water | In-house | N/A | |
| HEPES, 1M, pH 7.5 | Teknova | H1035 | |
| EGTA | Sigma | 03777-10 G | BCBW6026 |
| MgCl₂ | Sigma | M1028-100 mL | SLBP0426V |
| Brij-35 | EMD | 203728-50 mL | S159338 |
| DTT | Sigma | D5545-1 G | |
| Ovalbumin | Sigma | A2512-5 G | SLBT1992 |
| Tracer 222 | Invitrogen | PV6121 | 2066013B |
| DMSO | Sigma | D8418-100 ML | SHBG5286V |
| Tb-labeled inactive | In-house | N/A | |
| PDGFRβ | | | |
| 384 well plate | Greiner | 784075 | |
| Plate seal tape - iCycler | BioRad | 2239444 | |
| iQ optical tape | | | |

### II. Methods and Procedures

### Stock Solutions:

Assay buffer stock contains 50 mM HEPES pH7.5, 10 mM MgCl₂, 0.01% Brij-35, 1 mM EGTA.

Tb-labeled inactive PDGFRs. 3.6 μM in 50 mM HEPES, pH 7.4, 150 mM NaCl, 0.005% Tween-20 and 10% glycerol. Store at −80° C. in aliquots.

Tracer 222, 50 μM in DMSO, store at −20° C.

### Freshly Prepared Solutions:

Assay buffer. Add DTT to 2 mM and ovalbumin to 0.1 mg/mL to Assay buffer stock.

Kinase-Tracer solution. Make a working solution of 0.2 nM Tb-labeled inactive PDGFRβ and 40 nM Tracer 222 in Assay buffer. Keep on ice until use.

### Assay Procedure:

Step 1. Dispensing inhibitors: Using Echo, dispense 40 nL/well (or less) compound serial dilutions in DMSO onto the assay plate.

Step 2. Dispensing Kinase-Tracer solution: Add 4 μL/well Kinase-Tracer solution. Seal the plate with optically transparent plate seal. Centrifuge at 1000 rpm for 1 min.

Final concentrations of components in the assay:

[Tb-PDGFRβ]=0.2 nM;

[Tracer 222]=40 nM;

[DMSO]≤1%.

Step 3. Detection: Read TR-FRET signals after 18 hours incubation at room temperature.

### III. Calculations and Formulas

% Inhibition: % Inhibition=(NC−sample)/(NC−PC)*100 where NC is the mean of negative control (reactions without inhibitor), and PC is the mean of positive control (1 μM sunitinib).

IC₅₀ determination: Compounds are serially diluted 3-fold and tested in an 11-point dose response. IC₅₀ values are determined from a 4-parameter fit, using the following equation: Y=Bottom+(Top−Bottom)/(1+10((Log IC₅₀−X)*Hill slope)), where X=log₁₀ of the compound concentration; top can be defined by PC; bottom defined by NC. See Table 1, below.

### PDGFRβ Cellular Assay

### I. Materials

| Material Description | Vendor | Catalog |
|---|---|---|
| A10 Cells | ATCC | CRL-1476 |
| Rat Embryonic Aortic Smooth Muscle Cells | | |
| (ASMC) | | |
| Dulbecco′s Modified Eagle Medium (DMEM) | Gibco | 11965 |
| Fetal Bovine Serum (FBS) | Gibco | 16000 |
| Penicillin-Streptomycin (10,000 U/mL) (P/S) | Gibco | 15140 |
| PBS without Ca2+ and without Mg2+ | Corning | 21-031-CM |
| 0.25% Trypsin EDTA | Gibco | 25200 |
| Material Description | Vendor | Catalog |
| Platelet-derived Growth Factor (PDGF-BB); | R&D | 520-BB-050 |
| Rat | Systems | |
| PDGFRb P-Y751 Kit, 10,000 tests | CisBio | 64-PDGPEH |
| Greiner cell culture plate, poly-D-lysine, cell | Greiner | 781945 |
| coat 384 Well Plates | | |
| Dimethyl Sulfoxide (DMSO) | Sigma | MX1457 |

### II. Methods and Procedures

Cell Culture and Preparation: Cells are cultured according to ATCC procedure (5) with the addition of the antibiotic penicillin-streptomycin. If working from frozen, cells should be thawed according to ATCC procedure. Depending on the cell density of frozen vial, cells will need time to recover from thaw. An 80% confluent T75 flask should be enough for one 384 well plate.

Stock Solutions: Rat PDGFBB. A 100 ug/mL stock is prepared by reconstituting 50 ug in 500 μL of 4 mM HCl and 0.1% BSA. It can be stored for a month at 4° C., or aliquoted out and frozen in the −20/−80° C. to avoid multiple free-thaw cycles.

### Freshly Prepared Solutions:

1× Cisbio cell lysis buffer. The cell lysis buffer is diluted 4-fold with molecular grade water. The blocking agent is then diluted 25-fold in the diluted lysis buffer.

Antibody solutions. Equal amounts d2 and cryptate antibody are diluted 20-fold in detection buffer.

Rat PDGFBB. A working stock of 100 ng/mL is created from the stock solution in 10% FBS culture media.

### Assay Steps

Step 1: Plating Cells: Media from the A10 cell flask is aspirated. The cells are rinsed with PBS and then trypsinized to disperse the cell layer. The cells are then pelleted are resuspended to 1.25e5 cells/mL. 40 μL of cells are then plated in 384 Greiner TC treated plates at a density of 5000 cells/well using the Combi. Plates are covered and placed in the incubator (37° C. 5% CO₂) overnight to allow cells to adhere.

Step 2: Compound Dispense: Approximately 18 hours after plating, dispense 40 nL compound onto cells using Echo. Column 12 is the neutral control DMSO, column 24 is the inhibitor control 10 mM Imatinib (10 uM final assay concentration). The plate is returned to the incubator for 3 hours.

Step 3: Activation by PDGFbb: 6 μL of the working stock of 100 ng/mL PDGFbb is dispensed using the Tempest to give a final assay concentration of 15 ng/mL (EC80). After 10 minutes the media is removed by flicking the plate.

Step 4: Cell lysis and antibody addition: 20 uL lysis buffer per well is added to the plate via Tempest. 5 uL antibody solution is added per well via the Tempest. The plate is placed on the shaker at 230 rpm for 1 hour at room temperature.

Step 5: Detection: The plate is read using the HTRF module on the BMG Pherastar. Data is analyzed using Genedata Screener.

### III. Calculations and Formulas:

% Inhibition: % Inhibition=(NC−sample)/(NC−PC)*100 where NC is the mean of negative control (reactions without inhibitor), and PC is the mean of positive control (10 μM imatinib).

IC₅₀ determination: Compounds are serially diluted 3-fold and tested in an 11-point dose response. IC₅₀ values are determined from a 4-parameter fit, using the following equation: Y=Bottom+(Top−Bottom)/(1+10((Log IC₅₀−X)*Hill slope)), where X=log₁₀ of the compound concentration; top can be defined by PC; bottom defined by NC. See Table 1, below.

VEGFR ADP GLO assay

### I. Materials

| Reagent | Manufacturer | Catalog # | Lot # |
|---|---|---|---|
| MilliQ water | In-house | N/A | |
| HEPES, 1M, | Teknova | H1035 | |
| pH 7.5 | | | |
| EGTA | Sigma | 03777-10G | BCBW6026 |
| MgCl₂ | Sigma | M1028-100 mL | SLBP0426V |
| Brij-35 | EMD | 203728-50 mL | S159338 |
| DTT | Sigma | D5545-1 G | |
| Ovalbumin | Sigma | A2512-5 G | SLBT1992 |
| DMSO | Sigma | D8418-100 ML | SHBG5286V |
| VEGFR2, | Accelagen | N/A | EC_VEGFR2_03 |
| un- | | | Prep01A, |
| phosphorylated | | | Accelagen |
| | | | 20190321 |
| Srctide | Anaspec | AS-64105 | |
| ADP Glo | Promega | V9102 | |
| TAK-593 | MedChemExpress | HY-15506 | 36686 |
| Pluronic F-127 | Millipore | 540025-50 ML | |
| 384-well assay | Perkin Elmer | 6008289 | |
| plate (white, | | | |
| non-treated | | | |
| Proxiplate) | | | |
| 1536-well assay | Corning | 9146BC | |
| plate, (black, | | | |
| non-treated) | | | |

### II. Methods and Procedures

### Stock Solutions:

Assay buffer stock contains 50 mM HEPES pH7.5, 10 mM MgCl₂, 0.01% Brij-35, and 1 mM EGTA.

Unphosphorylated VEGFR2. 52.6 μM in 50 mM Tris-HCl, pH 8.0, 50 mM NaCl, 5% Glycerol, 0.5 mM TCEP. Store at −80° C. in aliquots.

10 mg/mL srctide solution, prepared in Assay buffer (Assay buffer stock with 2 mM DTT, 0.1% Pluronic F-127, and 0.1 mg/mL ovalbumin). Sonicate 10 mg/ml Srctide solution for 10 minutes and then vacuum filter.

### Freshly Prepared Solutions

Assay buffer. Add DTT to 2 mM, Pluronic F-127 to 0.1% and ovalbumin to 0.1 mg/mL to Assay buffer stock.

2× kinase solution. Make a working solution of 10 nM unphosphorylated VEGFR2 in Assay buffer. Vacuum filter 2× kinase solution prior to running assay. Keep on ice until use.

2× substrate/ATP solution. Make a working solution of 2 mg/mL srctide and 2.4 mM ATP in Assay buffer. Keep on ice until use.

### Assay Procedure:

Step 1. Dispensing inhibitors/controls: Using Echo, dispense 10 nL/well compound serial dilutions in DMSO to columns 1-22 (in 384-well plates) or columns 1-44 (in 1536-well plates). Dilution series=11 pt, 3-fold dilutions. The top compound concentration in the source plate is 4 mM. The top compound concentration in the assay plate is 10 uM. Using Echo, dispense 10 nl/well DMSO to column 23 (in 384-well plates) or columns 45-47 (in 1536-well plates). These wells will serve as negative control wells Using Echo, dispense 10 nl/well 400 uM TAK-593 in DMSO to column 24 (in 384-well plates) or column 48 (in 1536-well plates). The final concentration of TAK-593 in the assay should be 1 uM. These wells will serve as positive control wells.

Step 2. Pre-incubation of inhibitors with kinase: Add 2 μL/well 2× kinase solution. Centrifuge at 1000 rpm for 1 min. Incubate at room temperature for 30 min.

Step 3. Kinase cascade reaction: Add 2 μL/well 2× substrate/ATP solution to initiate kinase reactions. Centrifuge at 1000 rpm for 1 min. Incubate at room temperature for 180 min.

Final concentrations of components in the assay:

[VEGFR2]=5 nM;

[ATP]=1.2 mM;

[Srctide]=1 mg/mL;

[DMSO]≤1%.

Step 4. Quench: Add 2 uL/well ADP Glo Reagent+0.05% CHAPS. Centrifuge at 1000 rpm for 1 min; Incubate at room temperature for one hour.

Step 5. Detection: Add 2 uL/well Kinase Detection Reagent+0.05% CHAPS. Centrifuge at 1000 rpm for 1 min; Incubate at room temperature for 1 hour; Read Luminescence on a plate reader.

### III. Calculations and Formulas

% Inhibition: % Inhibition=(NC−sample)/(NC−PC)*100 where NC is the mean of negative control (reactions without inhibitor), and PC is the mean of positive control (1 μM TAK-593).

IC₅₀ determination: Compounds are serially diluted 3-fold and tested in an 11-point dose response. IC₅₀ values are determined from a 4-parameter fit, using the following equation: Y=Bottom+(Top−Bottom)/(1+10^{(Log IC50-X)*Hill Slope)}, where X=log₁₀ of the compound concentration; top can be defined by PC; bottom defined by NC. See Table 1, below.

TABLE 1
Biology chemical and cellular activity

| | PDGFR | PDGFR | VEGFR | PDGFR |
|---|---|---|---|---|
| Example | Lantha | HTRF | ADP GLO | Cellular |
| # | IC₅₀ (μM) | IC₅₀ (μM) | IC₅₀ (μM) | IC₅₀ (μM) |
| 1 | 0.0033 | 0.00065 | >7.500 | 0.0120 |
| 2 | 0.0037 | 0.00057 | >7.500 | 0.0271 |
| 3 | 0.0068 | 0.00068 | >7.500 | 0.1125 |
| 4 | 0.0032 | 0.00046 | >7.500 | 0.0145 |
| 5 | 0.0053 | 0.00065 | 2.073 | 0.0675 |
| 6 | 0.0052 | 0.00059 | >7.500 | 0.1019 |
| 7 | 0.0067 | 0.00048 | >7.500 | 0.0327 |
| 8 | 0.0058 | 0.00039 | >7.500 | 0.0480 |
| 9 | 0.0070 | 0.00059 | >7.500 | 0.0365 |
| 10 | 0.0099 | 0.00063 | >7.500 | 0.2038 |
| 11 | 0.0058 | 0.00068 | ~6.626 | 0.0688 |
| 12 | 0.0058 | 0.00048 | >7.500 | 0.1101 |
| 13 | 0.0128 | 0.00155 | >7.500 | 0.1517 |
| 14 | 0.0085 | 0.00089 | ~7.129 | 0.1090 |
| 15 | 0.0099 | 0.00101 | >7.500 | 0.0332 |
| 16 | 0.0185 | 0.00136 | >7.500 | 1.4781 |
| 17 | 0.0029 | 0.00048 | ~6.994 | 0.0683 |
| 18 | 0.0027 | 0.00051 | >7.500 | 0.0401 |
| 19 | 0.0045 | 0.00052 | >7.500 | 0.0560 |
| 20 | 0.0043 | 0.00051 | >7.500 | 0.1684 |
| 21 | 0.0034 | 0.00043 | >7.500 | 0.0663 |
| 22 | 0.0042 | 0.00040 | >7.500 | 0.0887 |
| 23 | 0.0027 | 0.00041 | >7.500 | 0.2942 |
| 24 | 0.0030 | 0.00055 | >7.500 | 0.0221 |
| 25 | 0.0027 | 0.00036 | >7.500 | 0.0129 |
| 26 | 0.0038 | 0.00057 | >7.500 | 0.0925 |
| 27 | 0.0040 | 0.00070 | >10 | 0.0312 |
| 28 | 0.0038 | 0.00050 | ~9.177 | 0.0394 |
| 29 | 0.0066 | 0.00080 | >10 | 5.3260 |
| 30 | 0.0368 | 0.0038 | >10 | >10 |
| 31 | 0.0603 | 0.0025 | >7.500 | 0.3899 |
| 32 | 0.0057 | 0.00054 | >7.500 | 0.1097 |
| 33 | 0.0088 | 0.00121 | 2.186 | >3.000 |
| 34 | 0.0213 | 0.00245 | >7.500 | 0.0317 |
| 35 | 0.0047 | 0.00068 | >7.500 | 1.6485 |
| 36 | 0.0883 | 0.00411 | >7.500 | >3.000 |
| 37 | 0.0065 | 0.00079 | 5.565 | 0.0642 |
| 38 | 0.0153 | 0.00159 | 0.715 | >3.000 |
| 39 | 0.0025 | 0.00030 | >7.500 | 0.0107 |
| 40 | 0.0130 | 0.00122 | >7.500 | 0.0141 |
| 41 | 0.0032 | 0.00036 | >7.500 | 0.1151 |
| 42 | 0.0039 | 0.00060 | >10 | 0.0332 |
| 43 | 0.0041 | 0.00054 | 2.084 | 0.0240 |
| 44 | 0.0097 | 0.00154 | 2.193 | 2.6583 |
| 45 | 1.6379 | 0.05096 | >10 | |
| 46 | 0.0049 | 0.00081 | 3.579 | 0.0185 |
| 47 | 0.0146 | 0.00258 | 2.423 | >3.000 |
| 48 | 0.0528 | 0.00107 | >7.500 | 0.1105 |
| 49 | 0.0402 | 0.00117 | >7.500 | 0.1053 |
| 50 | 0.0050 | 0.00025 | >7.500 | 0.0458 |
| 51 | 0.0090 | 0.00087 | >7.500 | 0.0172 |
| 52 | 0.1598 | 0.00471 | >2.500 | 0.0670 |
| 53 | 0.0031 | 0.00048 | 4.266 | 0.0062 |
| 54 | 0.0030 | 0.00043 | 4.727 | 0.0363 |
| 55 | 0.0095 | 0.00055 | >7.500 | 0.2752 |
| 56 | 0.0030 | 0.00054 | >7.500 | 0.0097 |
| 57 | 0.0056 | 0.00097 | >7.500 | 0.0156 |
| 58 | 0.0048 | 0.00036 | >7.500 | 0.1006 |
| 59 | 0.0062 | 0.00039 | >7.500 | 0.1901 |
| 60 | 0.0097 | 0.00065 | >7.500 | 0.0672 |
| 61 | 0.0102 | 0.00061 | 3.862 | 0.0868 |
| 62 | 0.0121 | 0.00083 | >7.500 | 0.0647 |
| 63 | 0.00089 | 0.00020 | 0.582 | 0.0016 |
| 64 | 0.0021 | 0.00026 | 1.032 | 0.0089 |
| 65 | 0.0021 | 0.00022 | 2.396 | 0.0100 |
| 66 | 0.0023 | 0.00028 | 1.328 | 0.0034 |
| 67 | 0.0019 | 0.00022 | 1.658 | 0.0098 |
| 68 | 0.0339 | 0.00198 | >7.500 | 0.1119 |
| 69 | 0.0484 | 0.00127 | >7.500 | 0.0369 |
| 70 | 0.0018 | 0.00020 | 6.166 | 0.0009 |
| 71 | 0.0021 | 0.00025 | >7.500 | 0.0017 |
| 72 | 0.0017 | 0.00027 | 1.0967 | 0.0035 |
| 73 | 0.0026 | 0.00050 | 4.570 | 0.0053 |
| 74 | 0.0016 | 0.00023 | 5.069 | 0.0017 |
| 75 | 0.0021 | 0.00035 | 0.847 | 0.0049 |
| 76 | 0.0026 | 0.00042 | 3.605 | 0.0131 |
| 77 | 0.0019 | 0.00024 | 5.106 | 0.0024 |
| 78 | 0.0037 | 0.00059 | 3.929 | 0.0083 |
| 79 | 0.0025 | 0.00041 | 5.386 | 0.0235 |
| 80 | 0.0032 | 0.00050 | >7.500 | 0.0166 |
| 81 | 0.0026 | 0.00042 | 2.499 | 0.0211 |
| 82 | 0.0017 | 0.00018 | >7.500 | 0.0202 |
| 83 | 0.0023 | 0.00029 | >7.500 | 0.0262 |
| 84 | 0.0036 | 0.00039 | >7.500 | 0.0111 |
| 85 | 0.0017 | 0.00027 | 6.602 | 0.0713 |
| 86 | 0.0022 | 0.00033 | >7.500 | 0.0182 |
| 87 | 0.0023 | 0.00029 | >7.500 | 0.0115 |
| 88 | 0.0030 | 0.00035 | >7.500 | 0.0223 |
| 89 | 0.0028 | 0.00033 | 4.092 | 0.0333 |
| 90 | 0.0018 | 0.00025 | >7.500 | 0.0272 |
| 91 | 0.0042 | 0.00046 | >7.500 | 0.0443 |
| 92 | 0.0043 | 0.00069 | 6.699 | 0.0228 |
| 93 | 0.0038 | 0.00044 | >7.500 | 0.0106 |
| 94 | 0.0021 | 0.00033 | 1.168 | 0.0139 |
| 95 | 0.0034 | 0.00053 | >7.500 | 0.0099 |
| 96 | 0.0028 | 0.00043 | >7.500 | 0.1395 |
| 97 | 0.0024 | 0.00046 | >7.500 | 0.1013 |
| 98 | 0.0120 | 0.00198 | >7.500 | 0.7228 |
| 99 | 0.0059 | 0.00066 | >7.500 | 0.1366 |
| 100 | 0.0028 | 0.00039 | 5.418 | 0.0931 |
| 101 | 0.0022 | 0.00038 | >7.500 | 0.0155 |
| 102 | 0.0038 | 0.00037 | >7.500 | 0.0076 |
| 103 | 0.0048 | 0.00055 | >7.5008 | 0.4432 |
| 104 | 0.0024 | 0.00044 | 0.0772 | 2.3589 |
| 105 | 0.0027 | 0.00059 | 3.3604 | 0.0097 |
| 106 | 0.0036 | 0.00076 | 0.915 | 0.0126 |
| 107 | 0.0028 | 0.00055 | 0.011 | >2.000 |
| 108 | >3.000 | 0.17207 | >7.500 | 2.2657 |
| 109 | 0.0040 | 0.00078 | 0.167 | 0.1269 |
| 110 | 0.0039 | 0.00077 | 0.143 | 0.1077 |
| 111 | 0.0051 | 0.00051 | >7.500 | 0.1064 |
| 112 | 0.0045 | 0.00057 | >7.500 | 0.0844 |
| 113 | 0.0068 | 0.00094 | >7.500 | 0.1159 |
| 114 | 0.0091 | 0.00088 | >7.500 | 0.0729 |
| 115 | 0.0039 | 0.00048 | >7.500 | 0.0206 |
| 116 | 0.0594 | 0.00263 | >7.500 | 0.721 |
| 117 | 0.1469 | 0.00428 | >7.500 | >2.000 |
| 118 | 0.0018 | 0.00029 | 0.076 | 0.0054 |
| 119 | 0.0014 | 0.00013 | 6.600 | 0.0058 |
| 120 | 0.0033 | 0.00032 | >7.500 | 0.0099 |
| 121 | 0.0013 | 0.00020 | >7.500 | 0.0036 |
| 122 | 0.0012 | 0.00015 | >7.500 | 0.0046 |
| 123 | 0.0015 | 0.00037 | >7.5007 | 0.0047 |
| 124 | 0.0016 | 0.00035 | 4.92833 | 0.0043 |
| 125 | 0.0017 | 0.00034 | >7.5007 | 0.0027 |
| 126 | 0.0021 | 0.00033 | 6.88335 | 0.0060 |
| 127 | 0.0014 | 0.00027 | 6.48784 | 0.0026 |
| 128 | 0.0036 | 0.00047 | >7.5007 | 0.0045 |
| 129 | 0.0013 | 0.00027 | 1.41775 | 0.0024 |
| 130 | 0.0015 | 0.00037 | >7.50067 | 0.0047 |
| 131 | 0.0016 | 0.00035 | 4.92833 | 0.0043 |
| 132 | 0.0020 | 0.00035 | >7.5007 | 0.0032 |
| 133 | 0.0024 | 0.00044 | 5.55009 | 0.0041 |
| 134 | 0.0031 | 0.00043 | >7.5007 | 0.0074 |
| 135 | 0.0028 | 0.00041 | >7.5007 | 0.0095 |
| 136 | 0.0037 | 0.00052 | >7.5007 | 0.0122 |
| 137 | 0.0045 | 0.00049 | >7.5007 | 0.0209 |
| 138 | 0.0021 | 0.00024 | >7.5007 | 0.0032 |
| 139 | 0.0028 | 0.00041 | >7.5007 | 0.0095 |
| 140 | 0.0016 | 0.00023 | >7.5007 | 0.0022 |
| 141 | 0.0125 | 0.00140 | >7.5007 | 0.0094 |
| 142 | 0.1066 | 0.00466 | >7.5007 | 0.0134 |
| 143 | 0.0117 | 0.00120 | >7.5007 | 0.0074 |
| 144 | 0.0028 | 0.00035 | >7.5007 | 0.0051 |
| 145 | 0.0153 | 0.00159 | >7.5007 | 0.0087 |
| 146 | 0.0197 | 0.00233 | >7.5007 | 0.0141 |
| 147 | 0.0021 | 0.00038 | 2.6699 | 0.0147 |
| 148 | 0.0043 | 0.00074 | >7.5007 | 0.0649 |
| 149 | 0.0022 | 0.00036 | 6.56448 | 0.0254 |
| 150 | 0.0012 | 0.00030 | 1.55275 | 0.0043 |
| 151 | 0.0017 | 0.00029 | 2.95529 | 0.0048 |
| 152 | 0.0034 | 0.00045 | 2.52581 | 0.0074 |
| 153 | 0.0013 | 0.00026 | >7.5007 | 0.1131 |
| 154 | 0.0120 | 0.00141 | >7.5007 | 0.1203 |
| 155 | 0.0155 | 0.00198 | >7.5007 | 0.1689 |
| 156 | 0.0202 | 0.00215 | >7.5007 | 0.0197 |
| 157 | 0.0329 | 0.00339 | >7.5007 | 0.1103 |
| 158 | 0.0223 | 0.00263 | >7.5007 | 0.1466 |
| 159 | 0.0133 | 0.00150 | >7.5007 | 0.0607 |
| 160 | 0.0113 | 0.00155 | >7.5007 | 0.0533 |
| 161 | 0.0133 | 0.00165 | >7.5007 | 0.0248 |
| 162 | 0.0155 | 0.00198 | >7.5007 | 0.1689 |
| 163 | 0.8048 | 0.08638 | >7.5007 | >1.9998 |

1. A compound of formula (I): 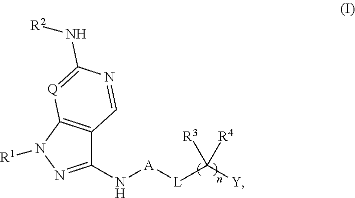
or a pharmaceutically acceptable salt thereof, wherein
A is an optionally substituted phenyl ring, an optionally substituted pyridinyl ring, or an optionally substituted 5-membered heteroaryl ring containing 1-2 heteroatoms that are each independently O, N, or S;
R¹ is an optionally substituted C₁-C₆ alkyl group;
R² is optionally substituted aryl, optionally substituted heteroaryl, optionally substituted cycloalkyl, optionally substituted C₁-C₆-alkyl-cycloalkyl, or optionally substituted heterocycloalkyl;
Q is N or CH;
n is 1, 2, or 3;
R³ and R⁴ are each independently H, C₁-C₆ alkyl, C₃-C₅ cycloalkyl,
or, when n is 2 or 3, one R³ and one R⁴ attached to the same carbon atom, together with that carbon atom, may form a C₃-C₆ cycloalkyl ring and one R³ and one R⁴ attached to the same carbon atom, together with that carbon atom, may form a carbonyl group (>C═O);
L is —C(O)NH—, —NHC(O)—, or —NHC(O)NH, wherein when n is 1 and Y is —NR⁵R⁶, then L is —NHC(O)—;
Y is substituted or unsubstituted 3-7 membered cycloalkyl, optionally substituted 4-7 membered heterocycloalkyl, or —NR⁵R⁶;
R⁵ and R⁶ are each independently optionally substituted aryl, optionally substituted heteroaryl, optionally substituted alkyl, optionally substituted cycloalkyl, or optionally substituted heterocycloalkyl; or R⁵ and R⁶, together with the nitrogen atom to which they are both attached, form an optionally substituted 3-12-membered heterocycloalkyl ring, an optionally substituted 5-12-membered bridged heterocycloalkyl ring, an optionally substituted 4-12-membered fused heterocycloalkyl ring system, or an optionally substituted 5-12-membered spiroheterocycloalkyl ring system, wherein said 3-12-membered heterocycloalkyl ring, 5-12-membered bridged heterocycloalkyl ring, 4-12-membered fused heterocycloalkyl ring system, or 5-12-membered spiroheterocycloalkyl ring system optionally includes, in addition to the nitrogen atom to which both R⁵ and R⁶ are attached, 1-3 other heteroatoms that are each independently O, S, or N.

2. The compound according to claim 1, wherein the compound of formula (I) is a compound of formula (IA) or formula (IB): 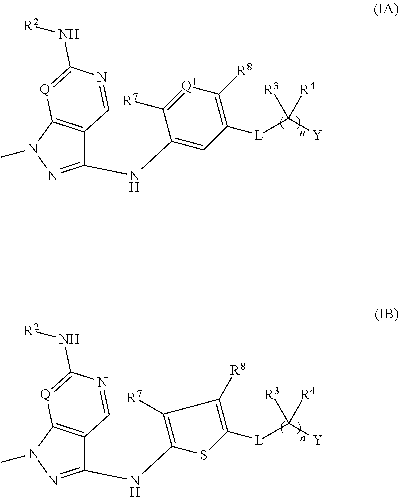
or a pharmaceutically acceptable salt thereof,
wherein
Q¹ is N or CH;
R⁷ is H, C₁-C₆ alkyl, C₃-C₆ cycloalkyl, halogen, —CN, or C₁-C₄ fluoroalkyl; and
R⁸ is H, C₁-C₆ alkyl, C₃-C₆ cycloalkyl, halogen, or C₁-C₄ fluoroalkyl.

3. The compound according to claim 1, wherein Q is N.

4. The compound according to claim 2, wherein said compound is a compound of formula (IA).

5.-8. (canceled)

9. The compound according to claim 4, wherein the compound of formula IA is a compound of formula IA-1:

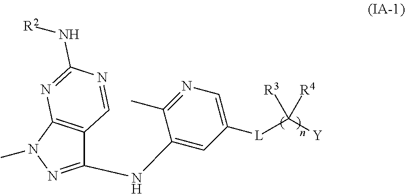

10. The compound according to claim 4, wherein the compound of formula IA is a compound of formula IA-2: 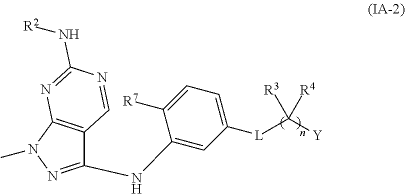
wherein R⁷ is —CH₃ or —F.

11. The compound according to claim 2, wherein said compound is a compound of formula (IB).

12. The compound according to claim 11, wherein the compound of formula IB is a compound of formula IB-1:

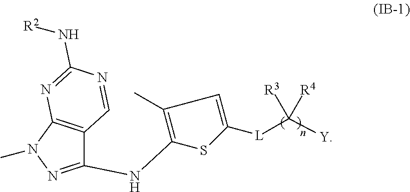

13.-22. (canceled)

23. The compound according to claim 1, wherein R³ and R⁴ are each H.

24. The compound according to claim 1, wherein an R³ and an R⁴ together with the carbon atom to which they are both attached, form a C₃-C₆ cycloalkyl ring.

25. The compound according to claim 1, wherein an R³ and an R⁴ together with the carbon atom to which they are both attached, form a carbonyl group (>C=O).

26. (canceled)

27. (canceled)

28. The compound according to claim 1, wherein R⁵ and R⁶ are each optionally substituted C₁-C₆ alkyl.

29. The compound according to claim 1, wherein R⁵ is optionally substituted C₁-C₆ alkyl and R⁶ is optionally substituted cycloalkyl or optionally substituted heterocycloalkyl.

30. The compound according to claim 1, wherein R⁵ and R⁶, together with the nitrogen atom to which they are both attached, (a) form an optionally substituted 3-12-membered heterocycloalkyl ring; or (b) form an optionally substituted 5-12-membered bridged heterocycloalkyl ring; or (c) form an optionally substituted 5-12-membered spiroheterocycloalkyl ring; or (d) form an optionally substituted 4-12-membered fused heterocycloalkyl ring system.

31. (canceled)

32. (canceled)

33. (canceled)

34. The compound according to claim 9, or a pharmaceutically acceptable salt thereof, wherein the compound is:
N-(2-(2,2-dimethylpyrrolidin-1-yl)ethyl)-6-methyl-5-((1-methyl-6-((1-methyl-1H-pyrazol-4-yl)amino)-1H-pyrazolo[3,4-d]pyrimidin-3-yl)amino)nicotinamide;
2-(3,3-dimethylazetidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide;
(S)—N-(6-methyl-5-((1-methyl-6-((1-methyl-1H-pyrazol-4-yl)amino)-1H-pyrazolo[3,4-d]pyrimidin-3-yl)amino)pyridin-3-yl)-2-(2-methylpyrrolidin-1-yl)acetamide;
2-(3,3-dimethylazetidin-1-yl)-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide;
N-(2-(2,2-dimethylpyrrolidin-1-yl)ethyl)-4-methyl-3-((1-methyl-6-((1-methyl-1H-pyrazol-4-yl)amino)-1H-pyrazolo[3,4-d]pyrimidin-3-yl)amino)benzamide;
(S)—N-(5-((6-((1-(2-methoxyethyl)-1H-pyrazol-4-yl)amino)-1-methyl-1H-pyrazolo[3, 4-d]pyrimidin-3-yl)amino)-6-methylpyridin-3-yl)-2-(2-methylpyrrolidin-1-yl)acetamide;
N-(2-(2,2-dimethylpyrrolidin-1-yl)ethyl)-6-methyl-5-((1-methyl-6-(pyrimidin-5-ylamino)-1H-pyrazolo[3,4-d]pyrimidin-3-yl)amino)nicotinamide;
N-(6-methyl-5-((1-methyl-6-((1-methyl-1H-pyrazol-4-yl)amino)-1H-pyrazolo[3,4-d]pyrimidin-3-yl)amino)pyridin-3-yl)-2-(5-azaspiro[2.4]heptan-5-yl)acetamide;
(S)—N-(6-methyl-5-((1-methyl-6-((1-methyl-1H-pyrazol-4-yl)amino)-1H-pyrazolo[3,4-d]pyrimidin-3-yl)amino)pyridin-3-yl)-2-(3-methylpyrrolidin-1-yl)acetamide;
2-(3,3-dimethylpyrrolidin-1-yl)-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide;
2-(2-azabicyclo[2.2.2]octan-2-yl)-N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)acetamide;
N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(5-azaspiro[3.4]octan-5-yl)acetamide;
N-(5-((6-((1-(2-methoxyethyl)-1H-pyrazol-4-yl)amino)-1-methyl-1H-pyrazolo[3,4-d]pyrimidin-3-yl)amino)-6-methylpyridin-3-yl)-2-(5-azaspiro[2.4]heptan-5-yl)acetamide;
(S)—N-(6-methyl-5-((1-methyl-6-((1-methyl-1H-pyrazol-4-yl)amino)-1H-pyrazolo[3,4-d]pyrimidin-3-yl)amino)pyridin-3-yl)-2-(3-methylpyrrolidin-1-yl)acetamide;
N-(2-(4-azaspiro[2.5]octan-4-yl)ethyl)-6-methyl-5-((1-methyl-6-((1-methyl-1H-pyrazol-4-yl)amino)-1H-pyrazolo[3,4-d]pyrimidin-3-yl)amino)nicotinamide;
2-(2,2-dimethylpyrrolidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide; or
2-(4,4-difluoropiperidin-1-yl)-N-(6-methyl-5-((1-methyl-6-((1-methyl-1H-pyrazol-4-yl)amino)-1H-pyrazolo[3,4-d]pyrimidin-3-yl)amino)pyridin-3-yl)acetamide.

35. A pharmaceutical composition comprising a compound according to claim 1, or a pharmaceutically acceptable salt thereof, and a pharmaceutically acceptable excipient.

36. A method of treating a disease or disorder in which PDGFR signaling is implicated in a subject in need thereof, comprising administering to said subject an amount of a compound according to claim 1, or a pharmaceutically acceptable salt thereof, that is effective to treat said disease or disorder.

37. The method according to claim 36, wherein said disease or disorder is pulmonary hypertension (PH).

38. The method according to claim 37, wherein said pulmonary hypertension is pulmonary arterial hypertension (PAH); PH secondary to heart failure; PH secondary to lung diseases and/or hypoxia; PH due to pulmonary artery obstruction; or PH due to unknown or rare diseases.

39. The method according to claim 38, wherein said pulmonary hypertension is pulmonary arterial hypertension (PAH).

40.-48. (canceled)
